(19) United States
(12) Patent Application Publication
(10) Pub. No.: US 2026/0096277 A1
(43) Pub. Date: Apr. 2, 2026

(54) HETEROCYCLIC COMPOUND AND ORGANIC LIGHT-EMITTING DEVICE INCLUDING SAME

(71) Applicant: LT MATERIALS CO., LTD., Yongin-si, Gyeonggi-do (KR)

(72) Inventors: Na-Yeong KIM, Yongin-si (KR); Su-Yeon KIM, Yongin-si (KR); Woo-Sam KIM, Yongin-si (KR); Won-Jang JEONG, Yongin-si (KR); Dong-Jun KIM, Yongin-si (KR); Dae-Hyuk CHOI, Yongin-si (KR)

(73) Assignee: LT MATERIALS CO., LTD., Yongin-si, Gyeonggi-do (KR)

(21) Appl. No.: 19/110,148

(22) PCT Filed: Jul. 11, 2023
(86) PCT No.: PCT/KR2023/009898

§ 371 Date: Mar. 10, 2025

(30) Foreign Application Priority Data
Oct. 18, 2022 (KR) 10-2022-0134269

(51) Int. Cl. H10K 50/11; C07B 59/00; C07D 307/91; C07D 333/76; H10K 50/15; H10K 85/60

(52) U.S. Cl. CPC H10K 50/11; C07B 59/004; C07D 307/91; C07D 333/76; H10K 50/15; H10K 85/622; H10K 85/626; H10K 85/633; H10K 85/636; H10K 85/6574; H10K 85/6576; C07B 2200/05

(57) ABSTRACT

The present specification relates to a heterocyclic compound and an organic light emitting device including the same.

### TECHNICAL FIELD

This application claims priority to and the benefit of Korean Patent Application No. 10-2022-0134269 filed in the Korean Intellectual Property Office on Oct. 18, 2022, the entire contents of which are incorporated herein by reference.

The present specification relates to a heterocyclic compound and an organic light emitting device including the same.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

U.S. Pat. No. 4,356,429

### DETAILED DESCRIPTION OF THE INVENTION

### Technical Problem

The present invention has been made in an effort to provide a heterocyclic compound and an organic light emitting device including the same.

### Technical Solution

In an exemplary embodiment of the present application, provided is a heterocyclic compound represented by the following Chemical Formula 1.

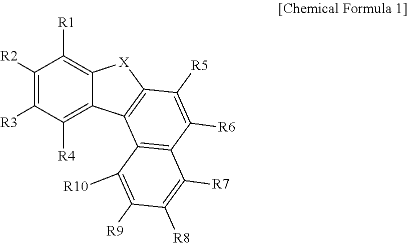

In Chemical Formula 1,
X is O; or S,
R1 to R10 are the same as or different from each other, and are each independently represented by hydrogen; deuterium; the following Chemical Formula 2; or following Chemical Formula 3,
one of R3 and R4; and R5 are represented by the following Chemical Formula 2 or the following Chemical Formula 3,

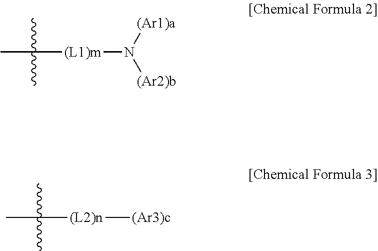

in Chemical Formulae 2 and 3,
L1 and L2 are the same as or different from each other, and are each independently a direct bond; a substituted or unsubstituted C6 to C60 arylene group; or a substituted or unsubstituted C2 to C60 heteroarylene group,
m and n are an integer from 0 to 4,
Ar1 to Ar3 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a, b, and c are an integer from 1 to 4, and
when m, n, a, b, and c are 2 or higher, substituents in the parenthesis are the same as or different from each other.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include one or more of the heterocyclic compound represented by Chemical Formula 1.

### Advantageous Effects

The compound described in the present specification can be used as a material for the organic material layer of the organic light emitting device. The compound may serve as a hole injection material, a hole transport material, a light emitting material, an electron transport material, an electron injection material, and the like in an organic light emitting device. In particular, the compound can be used as a material for a hole transport layer or hole auxiliary layer of an organic light emitting device.

Specifically, the compound is characterized in that one of R3 and R4; and R5 are represented by Chemical Formula 2 or Chemical Formula 3. When a compound having the substituent and substitution position as described above is used as a material for a hole transport layer in an organic light emitting device, an appropriate energy level and an appropriate band gap are formed to increase excitons in the light emitting region. Increasing excitons in the light emitting region means having an effect of reducing the driving voltage of the device and an effect of increasing efficiency.

In addition, as the compound has a substituent position as in Chemical Formula 1 of the present application, the substituted structure delocalizes the highest occupied molecular orbital (HOMO) energy level, thereby increasing the hole transport ability and stabilizing the HOMO energy.

### BRIEF DESCRIPTION OF DRAWINGS

FIGS. 1 to 3 each are views schematically illustrating a stacking structure of an organic light emitting device according to an exemplary embodiment of the present application.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

### BEST MODE

Hereinafter, the present specification will be described in more detail.

When one part “includes” one constituent element in the present specification, unless otherwise specifically described, this does not mean that another constituent element is excluded, but means that another constituent element may be further included.

In the present specification,

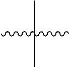

of a chemical formula means a position to which a constituent element is bonded.

In the present application, the term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; a cyano group; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or with a substituent to which two or more substituents selected among the exemplified substituents are bonded.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

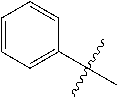 [SMILES: *c1ccccc1]

may be represented by 20% when the total number of substituents that the phenyl group can have is 5 (T1 in the formula) and the number of deuterium atoms among the substituents is 1 (T2 in the formula). That is, a deuterium content of 20% in the phenyl group may be represented by the following structural formula.

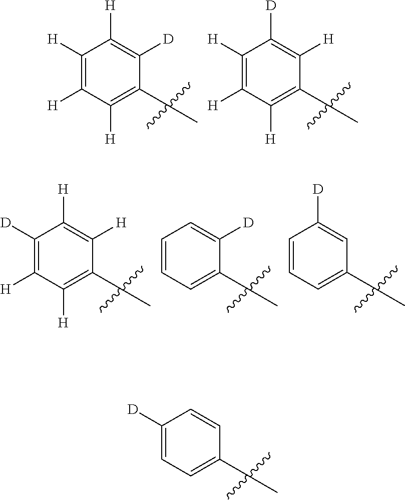

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes 0, S, Se, N, or Si as a heteroatom, includes a monocycle or polycycle having 2 to 60 carbon atoms, and may be additionally substituted with another substituent.

Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

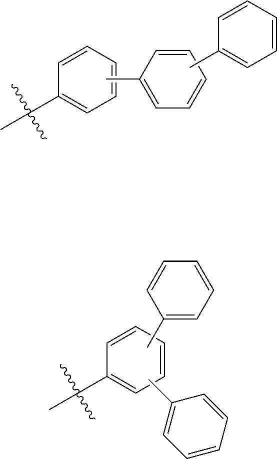

In the present specification, the fluorenyl group may be substituted, and adjacent substituents may be bonded to each other to form a ring.

When the fluorenyl group is substituted, the substituent may be the following structures, and the like, but is not limited thereto.

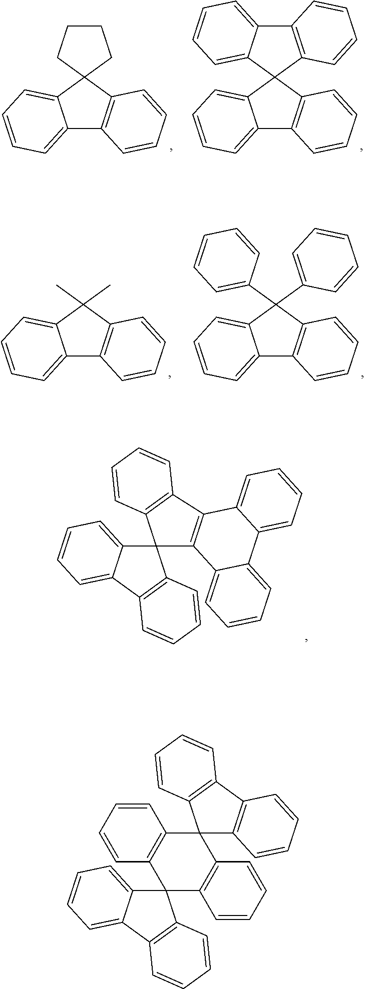

In the present specification, the heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, 2,3-dihydrobenzo[b]thiophene, 2,3-dihydrobenzofuran, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, a silyl group includes Si and is a substituent to which the Si atom is directly linked as a radical, and is represented by —Si(R101) (R102) (R103), and R101 to R103 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

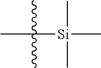 [SMILES: *[Si](C)(C)C]

(a trimethylsilyl group),

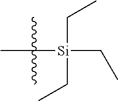 [SMILES: *[Si](CC)(CC)CC]

(a triethylsilyl group),

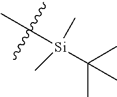 [SMILES: *[Si](C)(C)C(C)(C)C]

(a t-butyldimethylsilyl group),

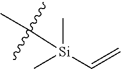 [SMILES: *[Si](C)(C)C=C]

(a vinyldimethylsilyl group),

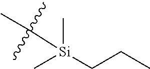 [SMILES: *[Si](C)(C)CCC]

(a propyldimethylsilyl group),

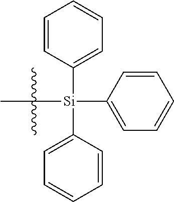 [SMILES: *[Si](c1ccccc1)(c1ccccc1)c1ccccc1]

(a triphenylsilyl group),

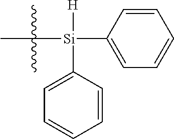 [SMILES: *[SiH](c1ccccc1)c1ccccc1]

(a diphenylsilyl group),

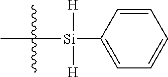 [SMILES: *[SiH2]c1ccccc1]

(a phenylsilyl group) and the like, but are not limited thereto.

In the present specification, the phosphine oxide group is represented by —P(═O) (R104) (R105), and R104 and R105 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, the amine group is represented by —N(R106) (R107), and R106 and R107 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

An exemplary embodiment of the present specification provides the heterocyclic compound represented by Chemical Formula 1.

In an exemplary embodiment of the present application, a group not represented by a substituent; or a group represented by hydrogen may mean being all substitutable with deuterium. That is, in an exemplary embodiment of the present application, it may be shown that hydrogen; or deuterium can be substituted with each other.

In an exemplary embodiment of the present application, X is O; or S.

In an exemplary embodiment of the present application, X is O.

In an exemplary embodiment of the present application, X is S.

In an exemplary embodiment of the present application, R1 to R10 are the same as or different from each other, and are each independently represented by hydrogen; deuterium; Chemical Formula 2; or Chemical Formula 3, and one of R3 and R4; and R5 may be represented by Chemical Formula 2 or Chemical Formula 3.

In an exemplary embodiment of the present application, Chemical Formula 1 may be represented by any one of the following Chemical Formulae 4 to 7.

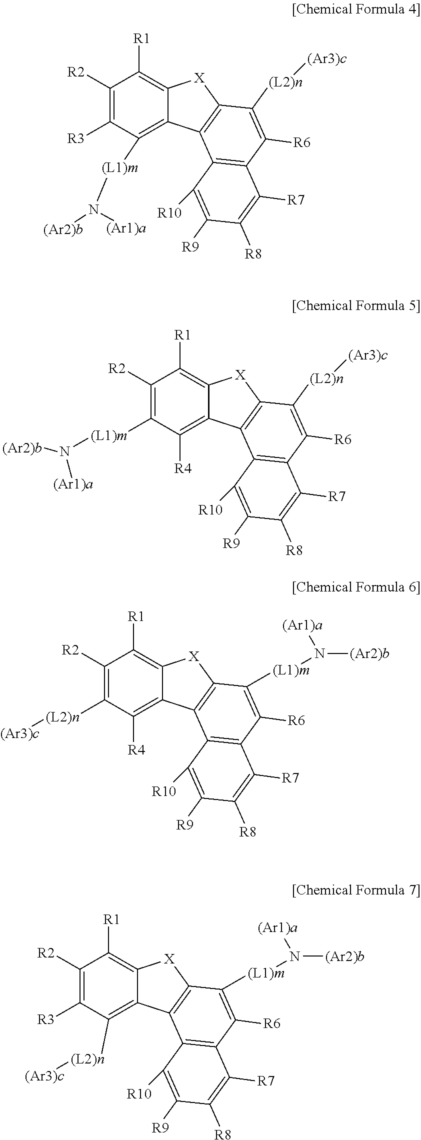

In Chemical Formulae 4 to 7,
the definitions of L1, L2, m, n, Ar1 to Ar3, X, a, b, and c are the same as the definitions in Chemical Formula 1, and
R1 to R4 and R6 to R10 are the same as or different from each other, and are each independently hydrogen; or deuterium.

In an exemplary embodiment of the present application, L1 and L2 are the same as or different from each other, and are each independently a direct bond; a substituted or unsubstituted C6 to C60 arylene group; or a substituted or unsubstituted C2 to C60 heteroarylene group.

In another exemplary embodiment, L1 and L2 are the same as or different from each other, and are each independently a direct bond; a substituted or unsubstituted C6 to C40 arylene group; or a substituted or unsubstituted C2 to C40 heteroarylene group.

In still another exemplary embodiment, L1 and L2 are the same as or different from each other, and are each independently a direct bond; a substituted or unsubstituted C6 to C20 arylene group; or a substituted or unsubstituted C2 to C20 heteroarylene group.

In yet another exemplary embodiment, L1 and L2 are the same as or different from each other, and are each independently a direct bond; a C6 to C20 arylene group; or a C2 to C20 heteroarylene group.

In still yet another exemplary embodiment, L1 and L12 are the same as or different from each other, and are each independently a direct bond; a substituted or unsubstituted phenylene group; a substituted or unsubstituted biphenylene group; or a substituted or unsubstituted naphthalene group.

In a further exemplary embodiment, L1 and L2 are the same as or different from each other, and are each independently a direct bond; a phenylene group; a biphenylene group; or a napthalene group.

In an exemplary embodiment of the present application, L1 and L2 may be unsubstituted or substituted with deuterium.

In an exemplary embodiment of the present application, Ar1 to Ar3 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, Ar1 to Ar3 are the same as or different from each other, and are each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, Ar1 to Ar3 are the same as or different from each other, and are each independently a substituted or unsubstituted C1 to C40 alkyl group; a substituted or unsubstituted C6 to C40 aryl group; or a substituted or unsubstituted C2 to C40 heteroaryl group.

In yet another exemplary embodiment, Ar1 to Ar3 are the same as or different from each other, and are each independently a substituted or unsubstituted C1 to C20 alkyl group; a substituted or unsubstituted C6 to C20 aryl group; or a substituted or unsubstituted C2 to C20 heteroaryl group.

In still yet another exemplary embodiment, Ar1 to Ar3 are the same as or different from each other, and are each independently a C1 to C20 alkyl group; a C6 to C20 aryl group unsubstituted or substituted with a C6 to C20 aryl group; or a C2 to C20 heteroaryl group unsubstituted or substituted with a C6 to C20 aryl group.

In a further exemplary embodiment, Ar1 to Ar3 are the same as or different from each other, and are each independently a substituted or unsubstituted phenyl group; a substituted or unsubstituted biphenyl group; a substituted or unsubstituted naphthyl group; a substituted or unsubstituted terphenyl group; a substituted or unsubstituted phenanthrene group; a substituted or unsubstituted triphenylenyl group; a substituted or unsubstituted dimethylfluorenyl group; a substituted or unsubstituted spirobifluorenyl group; a substituted or unsubstituted diphenylfluorenyl group; a substituted or unsubstituted dibenzofuran group; or a substituted or unsubstituted dibenzothiophene group.

In another further exemplary embodiment, Ar1 to Ar3 are the same as or different from each other, and are each independently a phenyl group unsubstituted or substituted with a phenyl group or a phenanthrene group; a biphenyl group unsubstituted or substituted with a phenyl group; a naphthyl group; a terphenyl group; a phenanthrene group; a triphenylenyl group; a dimethylfluorenyl group; a spirobifluorenyl group; a diphenylfluorenyl group; a dibenzofuran group; or a dibenzothiophene group.

In an exemplary embodiment of the present application, Ar1 to Ar3 may be unsubstituted or substituted with deuterium.

In an exemplary embodiment of the present application, Chemical Formula 2 may be represented by any one of the following Chemical Formulae 2-1 to 2-3.

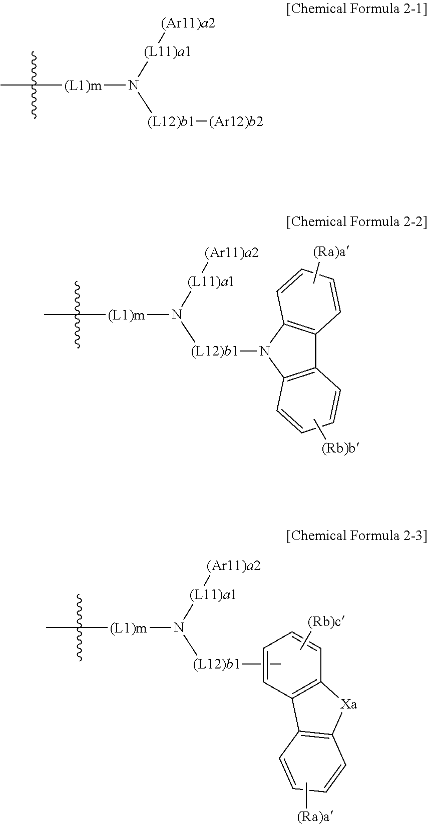

In Chemical Formulae 2-1 to 2-3,
the definitions of L1 and m are the same as the definitions in Chemical Formula 2,
L11 and L12 are the same as or different from each other, and are each independently a direct bond; a substituted or unsubstituted C6 to C60 arylene group; or a substituted or unsubstituted C2 to C60 heteroarylene group,
a1 and b1 are the same as or different from each other, and are each independently an integer from 0 to 3,
a2 and b2 are the same as or different from each other, and are each independently an integer from 1 to 3,
Ar11 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
Ar12 is a substituted or unsubstituted C6 to C60 aryl group,
Xa is O; S; or NRc,
Ra, Rb, and Rc are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a′ and b′ are an integer from 0 to 4,
c′ is an integer from 0 to 3, and
when a′, b′, c′, a1, a2, b2, and b1 are 2 or higher, substituents in the parenthesis are the same as or different from each other.

In an exemplary embodiment of the present application, L11 and L12 are the same as or different from each other, and are each independently a direct bond; a substituted or unsubstituted C6 to C60 arylene group; or a substituted or unsubstituted C2 to C60 heteroarylene group.

In another exemplary embodiment, L11 and L12 are the same as or different from each other, and are each independently a direct bond; a substituted or unsubstituted C6 to C40 arylene group; or a substituted or unsubstituted C2 to C40 heteroarylene group.

In still another exemplary embodiment, L11 and L12 are the same as or different from each other, and are each independently a direct bond; a substituted or unsubstituted C6 to C20 arylene group; or a substituted or unsubstituted C2 to C20 heteroarylene group.

In yet another exemplary embodiment, L11 and L12 are the same as or different from each other, and are each independently a direct bond; a C6 to C20 arylene group; or a C2 to C20 heteroarylene group.

In still yet another exemplary embodiment, L11 and L12 are the same as or different from each other, and are each independently a direct bond; a substituted or unsubstituted phenylene group; a substituted or unsubstituted biphenylene group; or a substituted or unsubstituted naphthalene group.

In a further exemplary embodiment, L11 and L12 are the same as or different from each other, and are each independently a direct bond; a phenylene group; a biphenylene group; or a naphthalene group.

In an exemplary embodiment of the present application, L11 and L12 may be unsubstituted or substituted with deuterium.

In an exemplary embodiment of the present application, Ar11 may be the same as the definition of the above-described Ar1.

In an exemplary embodiment of the present application, Ar12 is a substituted or unsubstituted C6 to C60 aryl group.

In an exemplary embodiment, Ar12 is a substituted or unsubstituted C6 to C40 aryl group.

In another exemplary embodiment, Ar12 is a substituted or unsubstituted C6 to C20 aryl group.

In still another exemplary embodiment, Ar12 is a C6 to C20 aryl group.

In yet another exemplary embodiment, Ar12 is independently a substituted or unsubstituted phenyl group; a substituted or unsubstituted biphenyl group; a substituted or unsubstituted naphthyl group; a substituted or unsubstituted terphenyl group; a substituted or unsubstituted phenanthrene group; a substituted or unsubstituted triphenylenyl group; a substituted or unsubstituted dimethylfluorenyl group; a substituted or unsubstituted spirobifluorenyl group; or a substituted or unsubstituted diphenylfluorenyl group.

In an exemplary embodiment of the present application, Ra, Rb, and Rc are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, Ra, Rb, and Rc are the same as or different from each other, and are each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, Ra, Rb, and Rc are the same as or different from each other, and are each independently hydrogen; deuterium; a substituted or unsubstituted C6 to C40 aryl group; or a substituted or unsubstituted C2 to C40 heteroaryl group.

In yet another exemplary embodiment, Ra, Rb and Rc are the same as or different from each other, and are each hydrogen; deuterium; a C6 to C40 aryl group; or a C2 to C40 heteroaryl group.

In still yet another exemplary embodiment, Ra, Rb, and Rc are the same as or different from each other, and may be each independently hydrogen; deuterium; or a substituted or unsubstituted phenyl group.

In further exemplary embodiment, Ra, Rb, and Rc are the same as or different from each other, and may be each independently hydrogen; deuterium; or a phenyl group.

In an exemplary embodiment of the present application, the deuterium content of the heterocyclic compound of Chemical Formula 1 may be 0% to 100%.

In another exemplary embodiment, the deuterium content of the heterocyclic compound of Chemical Formula 1 may be 0% or 3% to 100%, 5% to 100%, 7% to 100%, 10% to 100%, 15% to 100%, or 20% to 100%.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy. In addition, the bond length of carbon and deuterium is shorter than that of a bond with hydrogen, and a dissociation energy used to break the bond is also stronger than that of the bond with hydrogen. This is because the van der Waals radius of deuterium is smaller than that of hydrogen, and thus the extension amplitude of a bond between carbon and deuterium becomes even narrower.

The deuterium-substituted compound in the heterocyclic compound of Chemical Formula 1 of the present invention is characterized in that the energy in the ground state is further lower than that of the hydrogen-substituted compound, and the shorter the bond length between carbon and deuterium is, the smaller the molecular hardcore volume is. Accordingly, the electrical polarizability may be reduced and the intermolecular interaction can be weakened, so that the volume of the device thin film may be increased. These characteristics induce an effect of lowering the crystallinity by creating the amorphous state of a thin film. Therefore, deuterium substitution in the heterocyclic compound of Chemical Formula 1 may be effective in improving the heat resistance of an OLED device, thereby improving the service life and driving characteristics.

In an exemplary embodiment of the present application, provided is a heterocyclic compound in which Chemical Formula 1 is represented by any one of the following compounds.

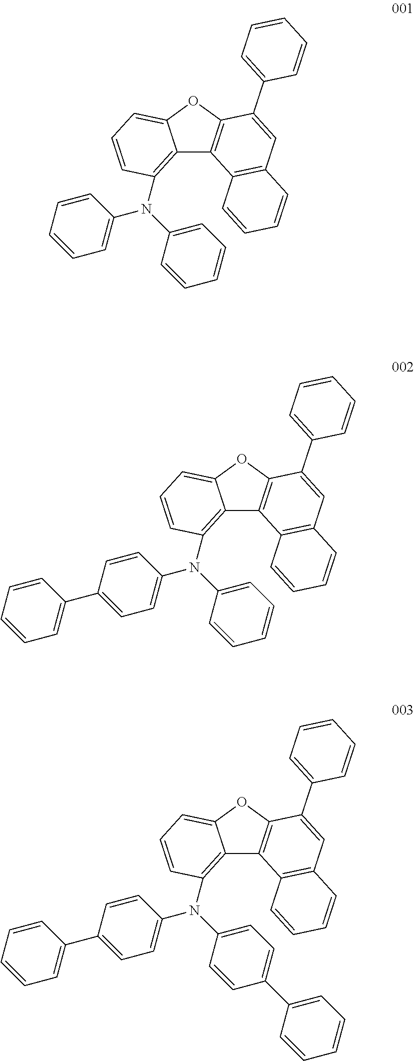 [SMILES: c1ccc(-c2cc3ccccc3c3c2oc2cccc(N(c4ccccc4)c4ccccc4)c23)cc1 ; c1ccc(-c2ccc(N(c3ccccc3)c3cccc4oc5c(-c6ccccc6)cc6ccccc6c5c34)cc2)cc1 ; c1ccc(-c2ccc(N(c3ccc(-c4ccccc4)cc3)c3cccc4oc5c(-c6ccccc6)cc6ccccc6c5c34)cc2)cc1]

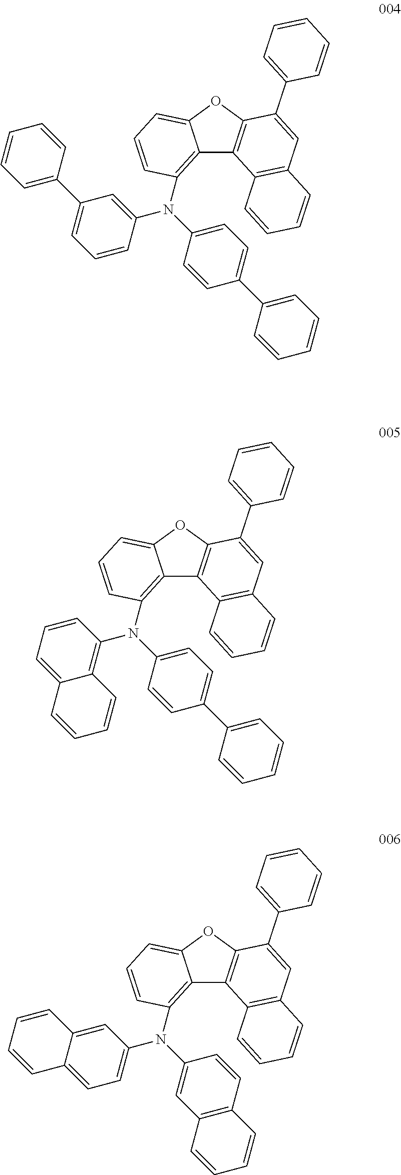 [SMILES: c1ccc(-c2ccc(N(c3cccc(-c4ccccc4)c3)c3cccc4oc5c(-c6ccccc6)cc6ccccc6c5c34)cc2)cc1 ; c1ccc(-c2ccc(N(c3cccc4ccccc34)c3cccc4oc5c(-c6ccccc6)cc6ccccc6c5c34)cc2)cc1 ; c1ccc(-c2cc3ccccc3c3c2oc2cccc(N(c4ccc5ccccc5c4)c4ccc5ccccc5c4)c23)cc1]

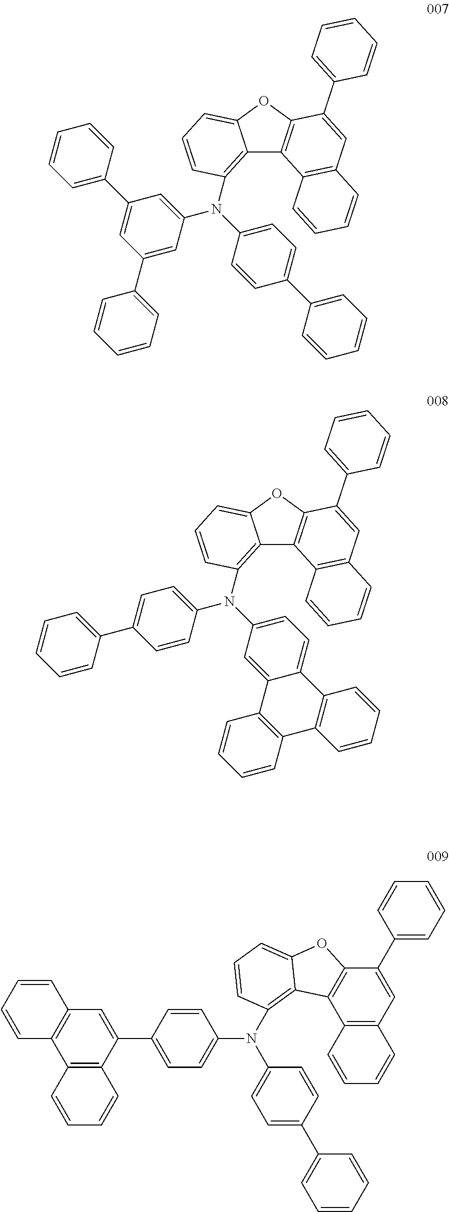 [SMILES: c1ccc(-c2ccc(N(c3cc(-c4ccccc4)cc(-c4ccccc4)c3)c3cccc4oc5c(-c6ccccc6)cc6ccccc6c5c34)cc2)cc1 ; c1ccc(-c2ccc(N(c3ccc4c5ccccc5c5ccccc5c4c3)c3cccc4oc5c(-c6ccccc6)cc6ccccc6c5c34)cc2)cc1 ; c1ccc(-c2ccc(N(c3ccc(-c4cc5ccccc5c5ccccc45)cc3)c3cccc4oc5c(-c6ccccc6)cc6ccccc6c5c34)cc2)cc1]

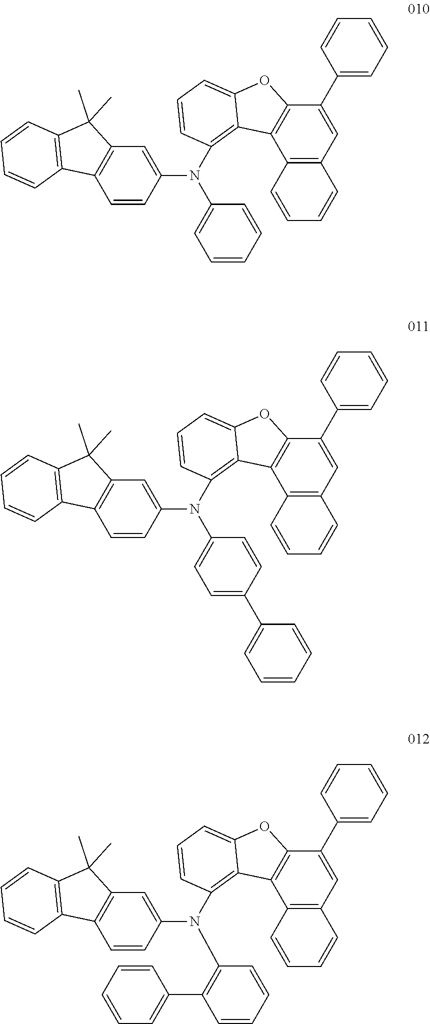 [SMILES: CC1(C)c2ccccc2-c2ccc(N(c3ccccc3)c3cccc4oc5c(-c6ccccc6)cc6ccccc6c5c34)cc21 ; CC1(C)c2ccccc2-c2ccc(N(c3ccc(-c4ccccc4)cc3)c3cccc4oc5c(-c6ccccc6)cc6ccccc6c5c34)cc21 ; CC1(C)c2ccccc2-c2ccc(N(c3ccccc3-c3ccccc3)c3cccc4oc5c(-c6ccccc6)cc6ccccc6c5c34)cc21]

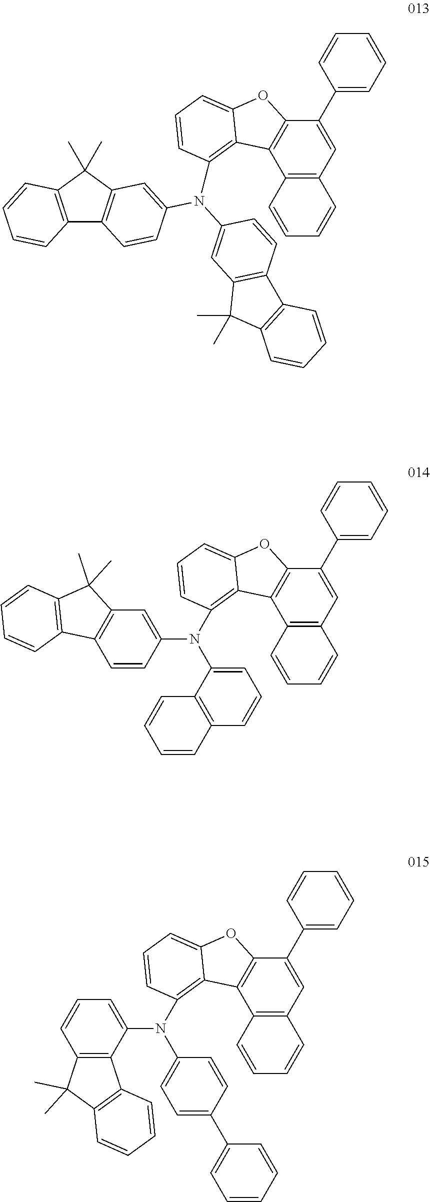 [SMILES: CC1(C)c2ccccc2-c2ccc(N(c3ccc4c(c3)C(C)(C)c3ccccc3-4)c3cccc4oc5c(-c6ccccc6)cc6ccccc6c5c34)cc21 ; CC1(C)c2ccccc2-c2ccc(N(c3cccc4ccccc34)c3cccc4oc5c(-c6ccccc6)cc6ccccc6c5c34)cc21 ; CC1(C)c2ccccc2-c2c(N(c3ccc(-c4ccccc4)cc3)c3cccc4oc5c(-c6ccccc6)cc6ccccc6c5c34)cccc21]

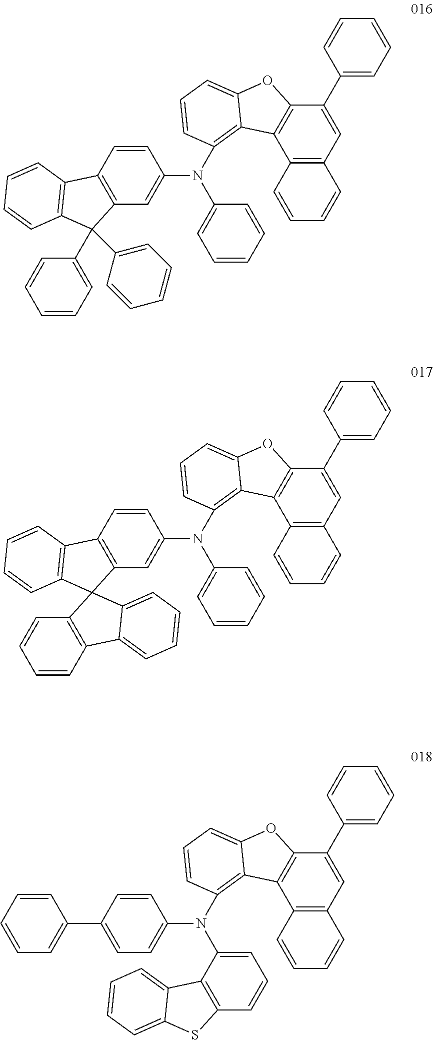 [SMILES: c1ccc(-c2cc3ccccc3c3c2oc2cccc(N(c4ccccc4)c4ccc5c(c4)C(c4ccccc4)(c4ccccc4)c4ccccc4-5)c23)cc1 ; c1ccc(-c2cc3ccccc3c3c2oc2cccc(N(c4ccccc4)c4ccc5c(c4)C4(c6ccccc6-c6ccccc64)c4ccccc4-5)c23)cc1 ; c1ccc(-c2ccc(N(c3cccc4sc5ccccc5c34)c3cccc4oc5c(-c6ccccc6)cc6ccccc6c5c34)cc2)cc1]

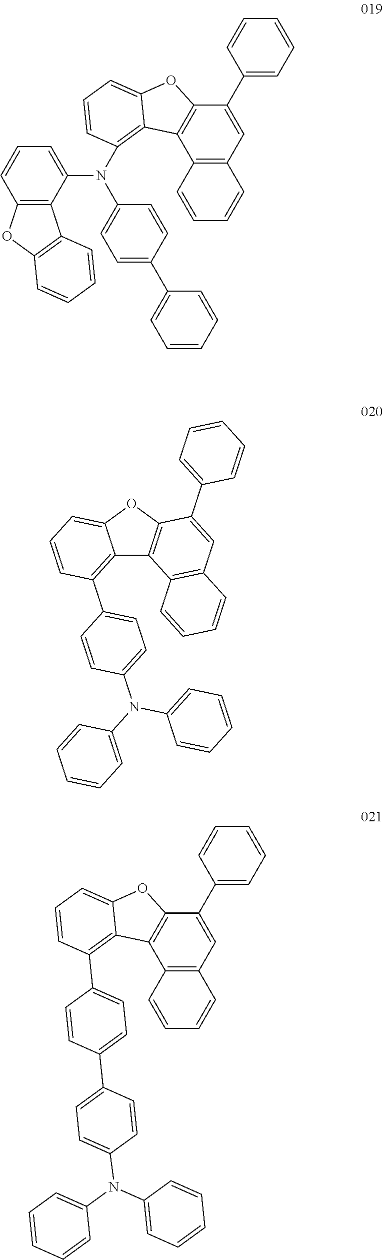 [SMILES: c1ccc(-c2ccc(N(c3cccc4oc5ccccc5c34)c3cccc4oc5c(-c6ccccc6)cc6ccccc6c5c34)cc2)cc1 ; c1ccc(-c2cc3ccccc3c3c2oc2cccc(-c4ccc(N(c5ccccc5)c5ccccc5)cc4)c23)cc1 ; c1ccc(-c2cc3ccccc3c3c2oc2cccc(-c4ccc(-c5ccc(N(c6ccccc6)c6ccccc6)cc5)cc4)c23)cc1]

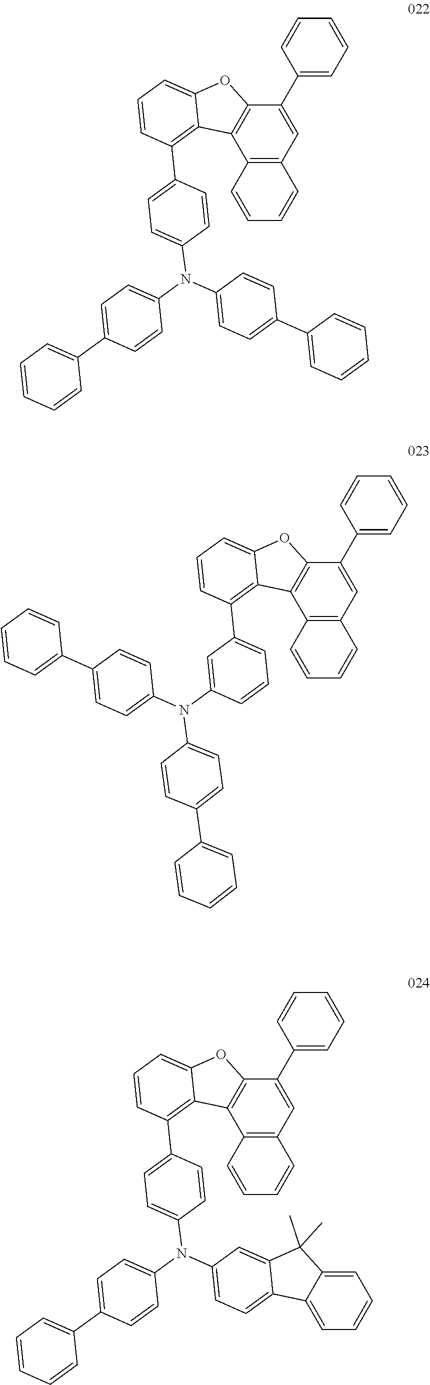 [SMILES: c1ccc(-c2ccc(N(c3ccc(-c4ccccc4)cc3)c3ccc(-c4cccc5oc6c(-c7ccccc7)cc7ccccc7c6c45)cc3)cc2)cc1 ; c1ccc(-c2ccc(N(c3ccc(-c4ccccc4)cc3)c3cccc(-c4cccc5oc6c(-c7ccccc7)cc7ccccc7c6c45)c3)cc2)cc1 ; CC1(C)c2ccccc2-c2ccc(N(c3ccc(-c4ccccc4)cc3)c3ccc(-c4cccc5oc6c(-c7ccccc7)cc7ccccc7c6c45)cc3)cc21]

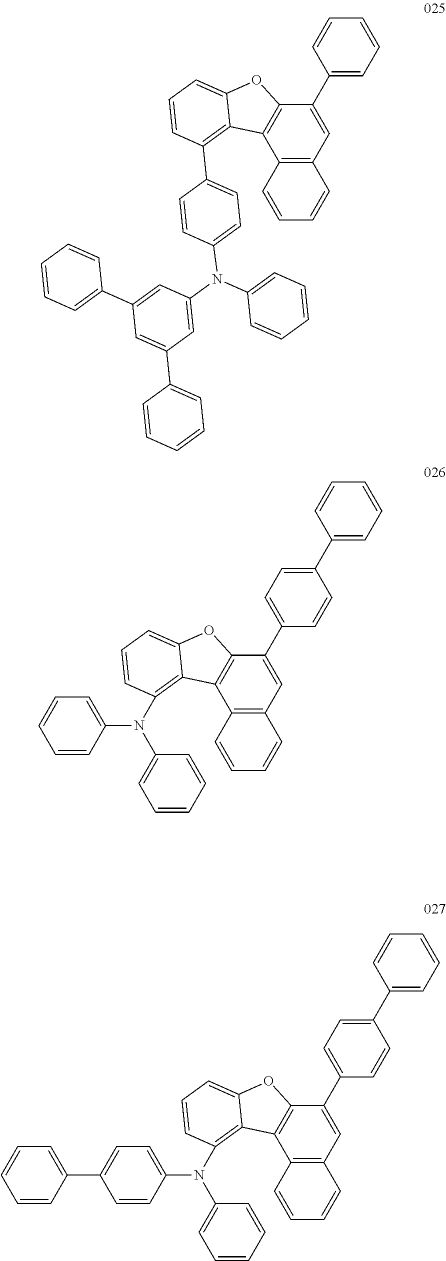 [SMILES: c1ccc(-c2cc(-c3ccccc3)cc(N(c3ccccc3)c3ccc(-c4cccc5oc6c(-c7ccccc7)cc7ccccc7c6c45)cc3)c2)cc1 ; c1ccc(-c2ccc(-c3cc4ccccc4c4c3oc3cccc(N(c5ccccc5)c5ccccc5)c34)cc2)cc1 ; c1ccc(-c2ccc(-c3cc4ccccc4c4c3oc3cccc(N(c5ccccc5)c5ccc(-c6ccccc6)cc5)c34)cc2)cc1]

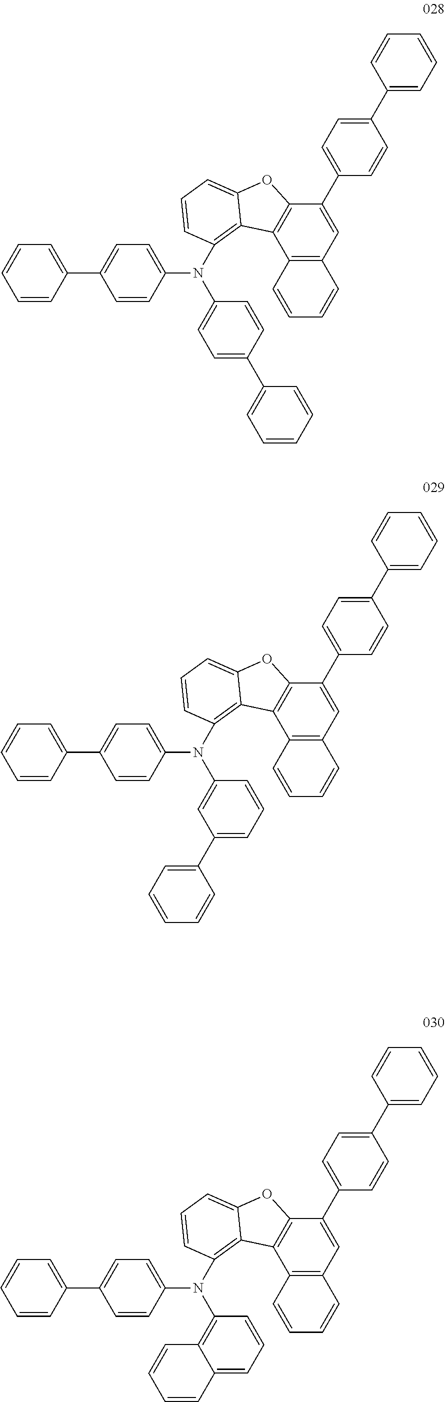 [SMILES: c1ccc(-c2ccc(-c3cc4ccccc4c4c3oc3cccc(N(c5ccc(-c6ccccc6)cc5)c5ccc(-c6ccccc6)cc5)c34)cc2)cc1 ; c1ccc(-c2ccc(-c3cc4ccccc4c4c3oc3cccc(N(c5ccc(-c6ccccc6)cc5)c5cccc(-c6ccccc6)c5)c34)cc2)cc1 ; c1ccc(-c2ccc(-c3cc4ccccc4c4c3oc3cccc(N(c5ccc(-c6ccccc6)cc5)c5cccc6ccccc56)c34)cc2)cc1]

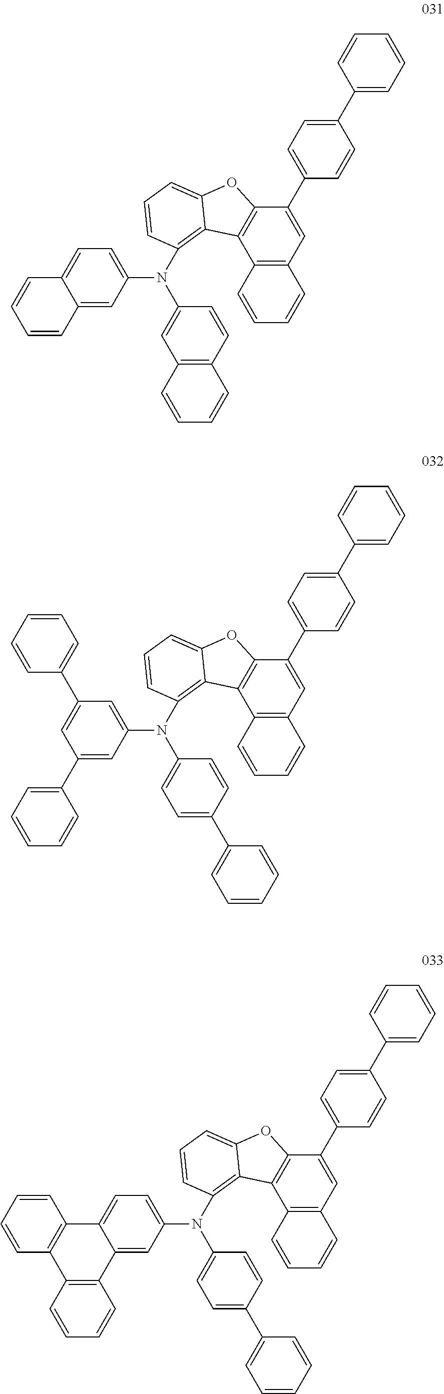 [SMILES: c1ccc(-c2ccc(-c3cc4ccccc4c4c3oc3cccc(N(c5ccc6ccccc6c5)c5ccc6ccccc6c5)c34)cc2)cc1 ; c1ccc(-c2ccc(-c3cc4ccccc4c4c3oc3cccc(N(c5ccc(-c6ccccc6)cc5)c5cc(-c6ccccc6)cc(-c6ccccc6)c5)c34)cc2)cc1 ; c1ccc(-c2ccc(-c3cc4ccccc4c4c3oc3cccc(N(c5ccc(-c6ccccc6)cc5)c5ccc6c7ccccc7c7ccccc7c6c5)c34)cc2)cc1]

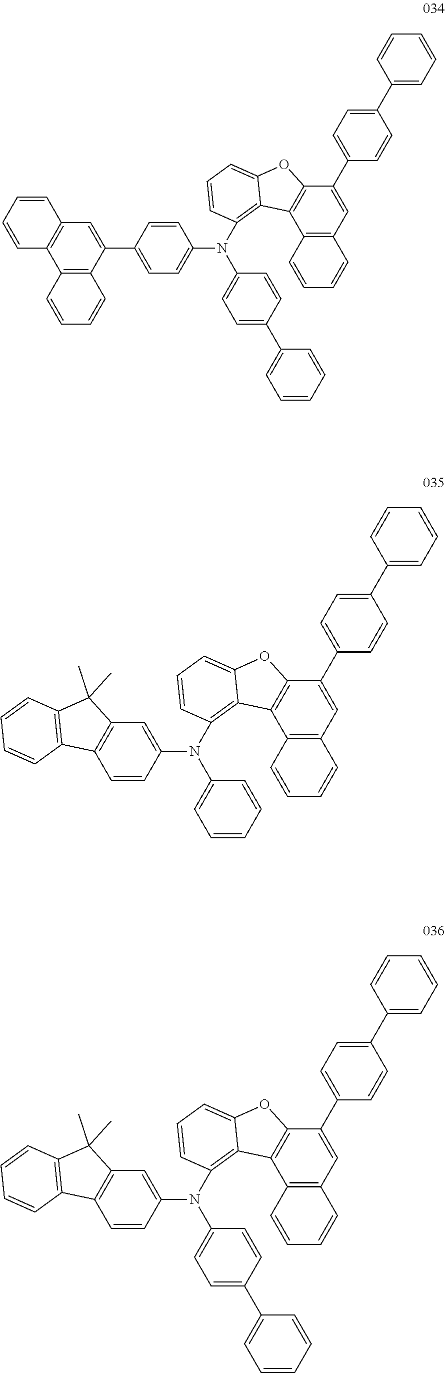 [SMILES: c1ccc(-c2ccc(-c3cc4ccccc4c4c3oc3cccc(N(c5ccc(-c6ccccc6)cc5)c5ccc(-c6cc7ccccc7c7ccccc67)cc5)c34)cc2)cc1 ; CC1(C)c2ccccc2-c2ccc(N(c3ccccc3)c3cccc4oc5c(-c6ccc(-c7ccccc7)cc6)cc6ccccc6c5c34)cc21 ; CC1(C)c2ccccc2-c2ccc(N(c3ccc(-c4ccccc4)cc3)c3cccc4oc5c(-c6ccc(-c7ccccc7)cc6)cc6ccccc6c5c34)cc21]

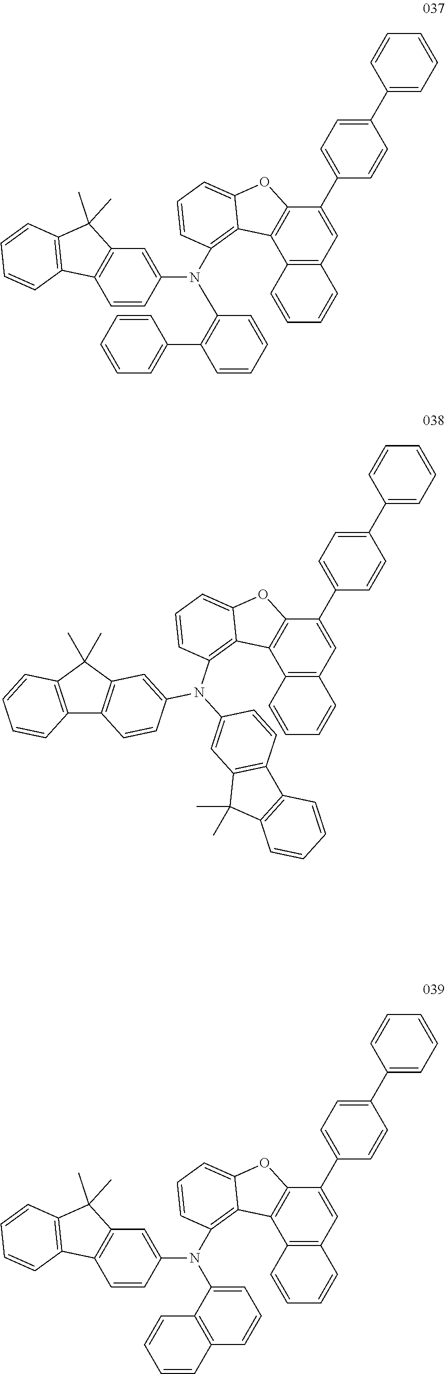 [SMILES: CC1(C)c2ccccc2-c2ccc(N(c3ccccc3-c3ccccc3)c3cccc4oc5c(-c6ccc(-c7ccccc7)cc6)cc6ccccc6c5c34)cc21 ; CC1(C)c2ccccc2-c2ccc(N(c3ccc4c(c3)C(C)(C)c3ccccc3-4)c3cccc4oc5c(-c6ccc(-c7ccccc7)cc6)cc6ccccc6c5c34)cc21 ; CC1(C)c2ccccc2-c2ccc(N(c3cccc4ccccc34)c3cccc4oc5c(-c6ccc(-c7ccccc7)cc6)cc6ccccc6c5c34)cc21]

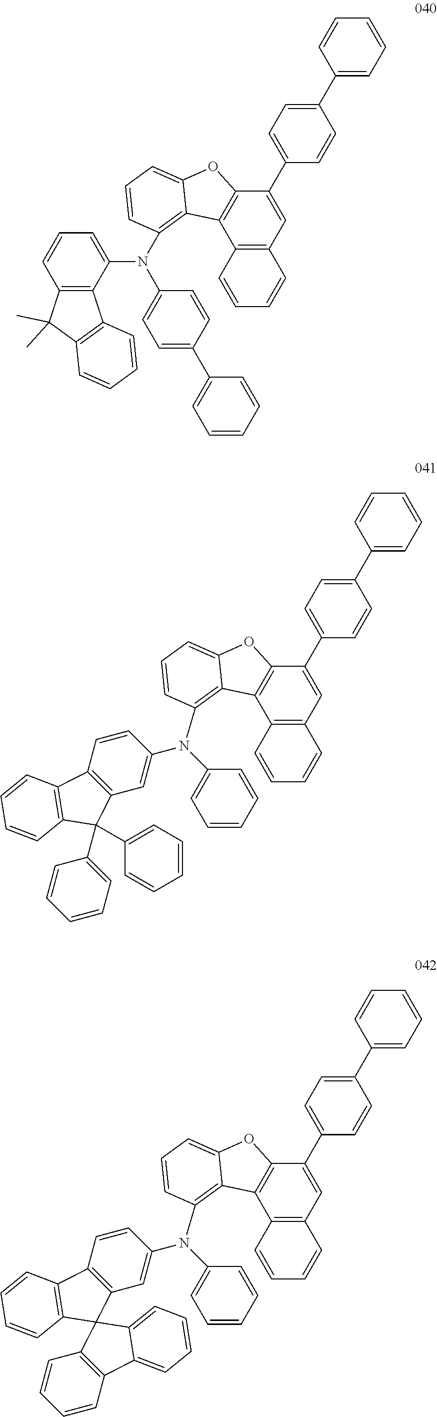 [SMILES: CC1(C)c2ccccc2-c2c(N(c3ccc(-c4ccccc4)cc3)c3cccc4oc5c(-c6ccc(-c7ccccc7)cc6)cc6ccccc6c5c34)cccc21 ; c1ccc(-c2ccc(-c3cc4ccccc4c4c3oc3cccc(N(c5ccccc5)c5ccc6c(c5)C(c5ccccc5)(c5ccccc5)c5ccccc5-6)c34)cc2)cc1 ; c1ccc(-c2ccc(-c3cc4ccccc4c4c3oc3cccc(N(c5ccccc5)c5ccc6c(c5)C5(c7ccccc7-c7ccccc75)c5ccccc5-6)c34)cc2)cc1]

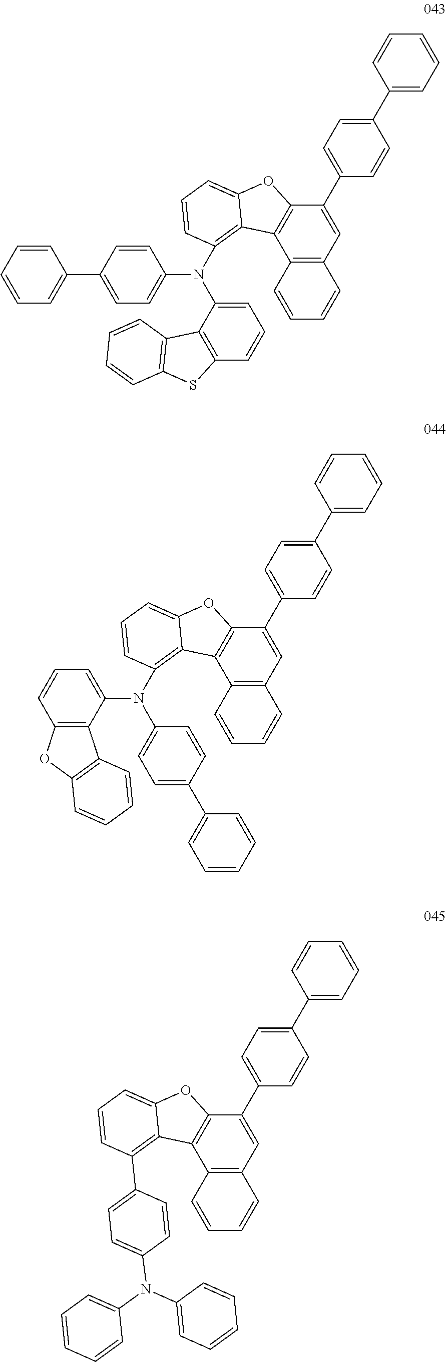 [SMILES: c1ccc(-c2ccc(-c3cc4ccccc4c4c3oc3cccc(N(c5ccc(-c6ccccc6)cc5)c5cccc6sc7ccccc7c56)c34)cc2)cc1 ; c1ccc(-c2ccc(-c3cc4ccccc4c4c3oc3cccc(N(c5ccc(-c6ccccc6)cc5)c5cccc6oc7ccccc7c56)c34)cc2)cc1 ; c1ccc(-c2ccc(-c3cc4ccccc4c4c3oc3cccc(-c5ccc(N(c6ccccc6)c6ccccc6)cc5)c34)cc2)cc1]

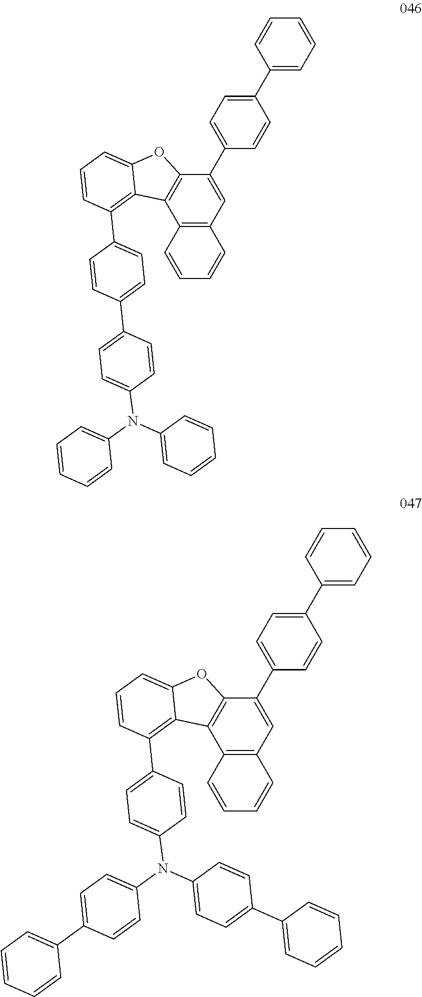 [SMILES: c1ccc(-c2ccc(-c3cc4ccccc4c4c3oc3cccc(-c5ccc(-c6ccc(N(c7ccccc7)c7ccccc7)cc6)cc5)c34)cc2)cc1 ; c1ccc(-c2ccc(-c3cc4ccccc4c4c3oc3cccc(-c5ccc(N(c6ccc(-c7ccccc7)cc6)c6ccc(-c7ccccc7)cc6)cc5)c34)cc2)cc1]

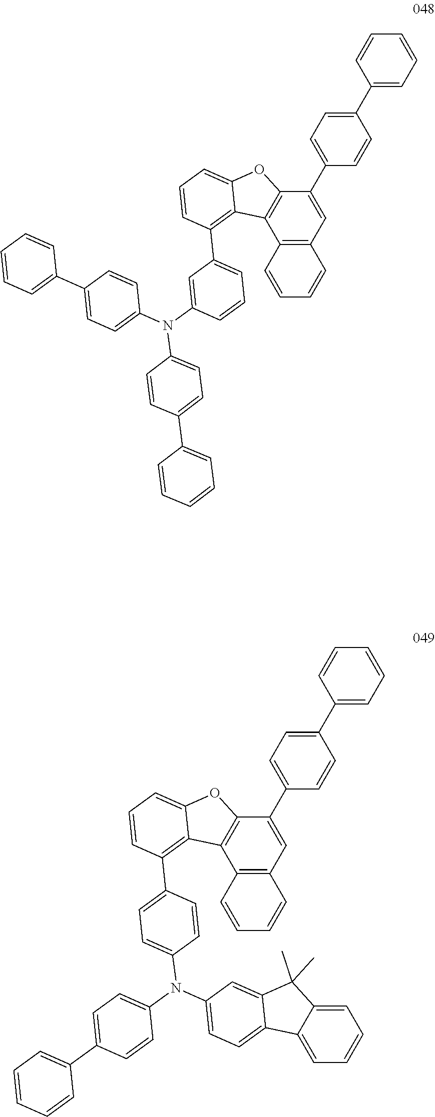 [SMILES: c1ccc(-c2ccc(-c3cc4ccccc4c4c3oc3cccc(-c5cccc(N(c6ccc(-c7ccccc7)cc6)c6ccc(-c7ccccc7)cc6)c5)c34)cc2)cc1 ; CC1(C)c2ccccc2-c2ccc(N(c3ccc(-c4ccccc4)cc3)c3ccc(-c4cccc5oc6c(-c7ccc(-c8ccccc8)cc7)cc7ccccc7c6c45)cc3)cc21]

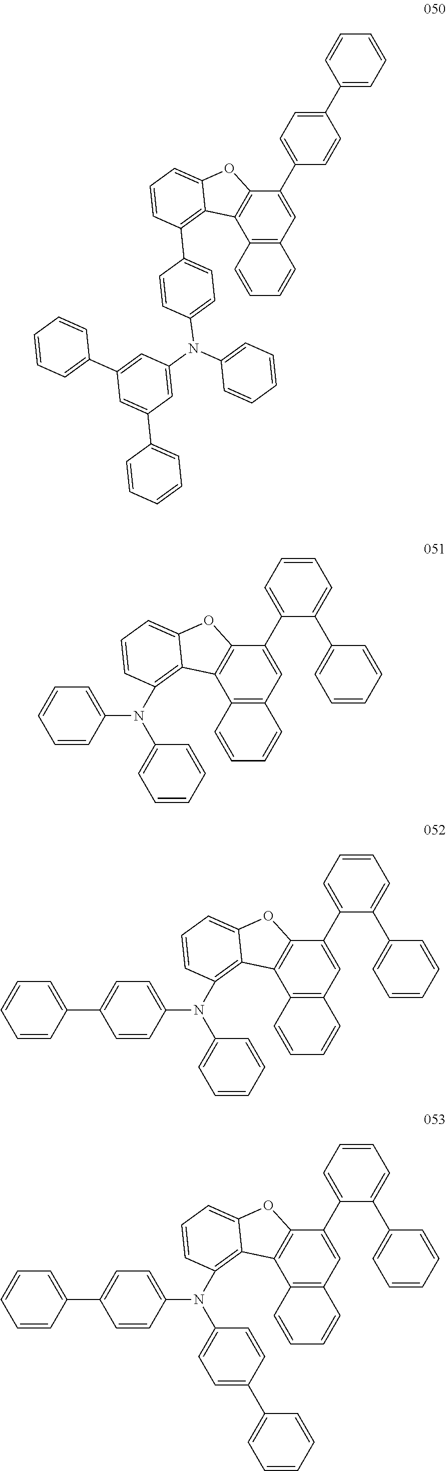 [SMILES: c1ccc(-c2ccc(-c3cc4ccccc4c4c3oc3cccc(-c5ccc(N(c6ccccc6)c6cc(-c7ccccc7)cc(-c7ccccc7)c6)cc5)c34)cc2)cc1 ; c1ccc(-c2ccccc2-c2cc3ccccc3c3c2oc2cccc(N(c4ccccc4)c4ccccc4)c23)cc1 ; c1ccc(-c2ccc(N(c3ccccc3)c3cccc4oc5c(-c6ccccc6-c6ccccc6)cc6ccccc6c5c34)cc2)cc1 ; c1ccc(-c2ccc(N(c3ccc(-c4ccccc4)cc3)c3cccc4oc5c(-c6ccccc6-c6ccccc6)cc6ccccc6c5c34)cc2)cc1]

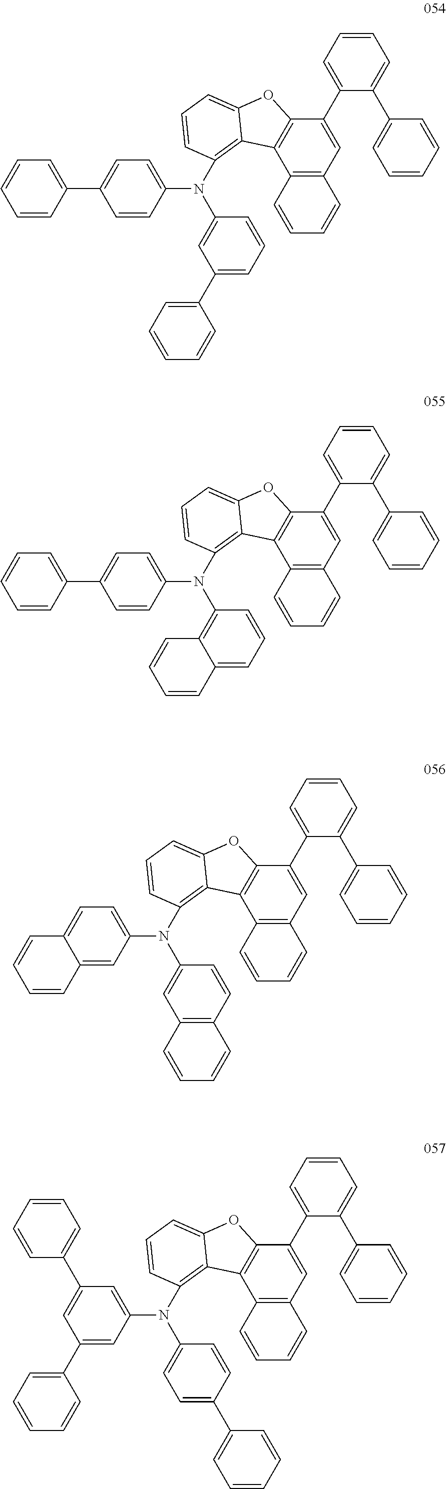 [SMILES: c1ccc(-c2ccc(N(c3cccc(-c4ccccc4)c3)c3cccc4oc5c(-c6ccccc6-c6ccccc6)cc6ccccc6c5c34)cc2)cc1 ; c1ccc(-c2ccc(N(c3cccc4ccccc34)c3cccc4oc5c(-c6ccccc6-c6ccccc6)cc6ccccc6c5c34)cc2)cc1 ; c1ccc(-c2ccccc2-c2cc3ccccc3c3c2oc2cccc(N(c4ccc5ccccc5c4)c4ccc5ccccc5c4)c23)cc1 ; c1ccc(-c2ccc(N(c3cc(-c4ccccc4)cc(-c4ccccc4)c3)c3cccc4oc5c(-c6ccccc6-c6ccccc6)cc6ccccc6c5c34)cc2)cc1]

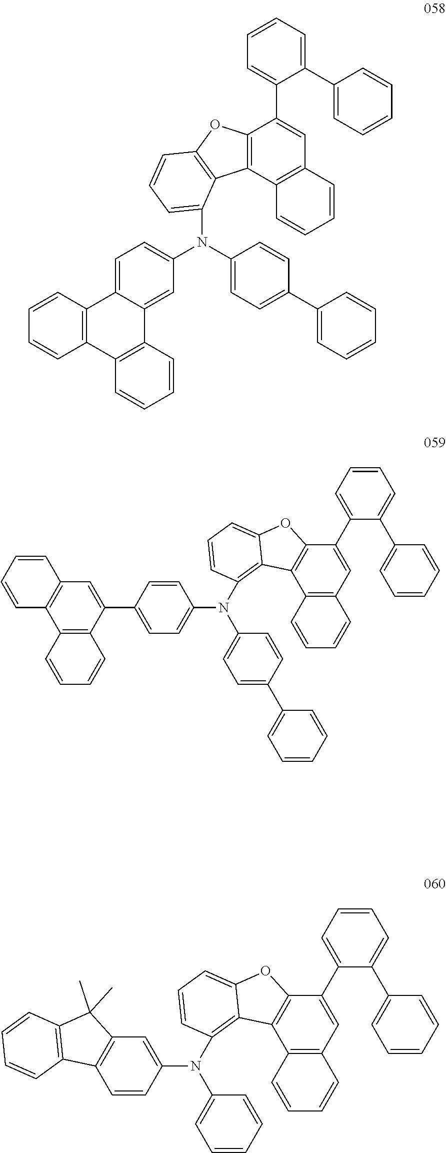 [SMILES: c1ccc(-c2ccc(N(c3ccc4c5ccccc5c5ccccc5c4c3)c3cccc4oc5c(-c6ccccc6-c6ccccc6)cc6ccccc6c5c34)cc2)cc1 ; c1ccc(-c2ccc(N(c3ccc(-c4cc5ccccc5c5ccccc45)cc3)c3cccc4oc5c(-c6ccccc6-c6ccccc6)cc6ccccc6c5c34)cc2)cc1 ; CC1(C)c2ccccc2-c2ccc(N(c3ccccc3)c3cccc4oc5c(-c6ccccc6-c6ccccc6)cc6ccccc6c5c34)cc21]

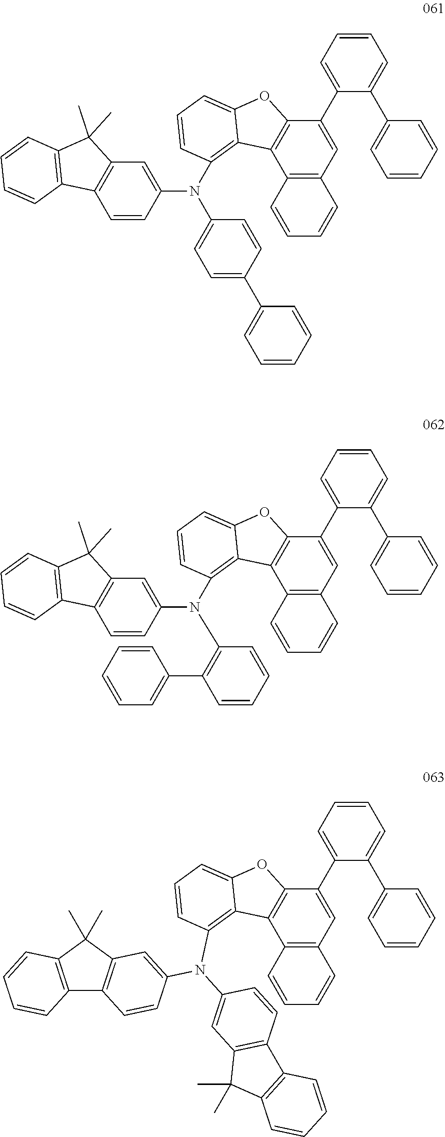 [SMILES: CC1(C)c2ccccc2-c2ccc(N(c3ccc(-c4ccccc4)cc3)c3cccc4oc5c(-c6ccccc6-c6ccccc6)cc6ccccc6c5c34)cc21 ; CC1(C)c2ccccc2-c2ccc(N(c3ccccc3-c3ccccc3)c3cccc4oc5c(-c6ccccc6-c6ccccc6)cc6ccccc6c5c34)cc21 ; CC1(C)c2ccccc2-c2ccc(N(c3ccc4c(c3)C(C)(C)c3ccccc3-4)c3cccc4oc5c(-c6ccccc6-c6ccccc6)cc6ccccc6c5c34)cc21]

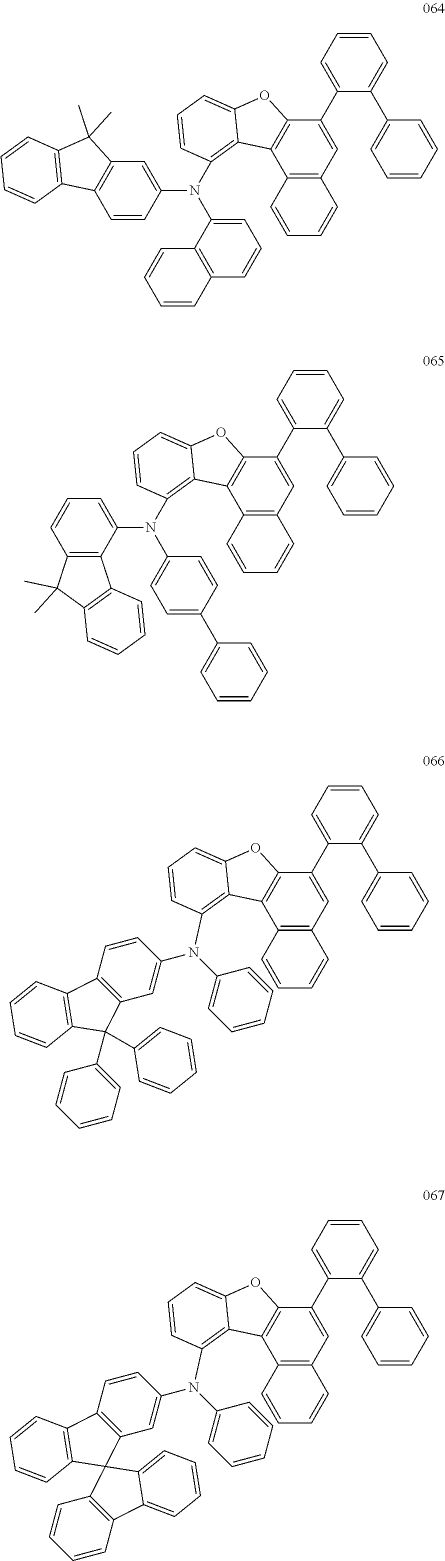 [SMILES: CC1(C)c2ccccc2-c2ccc(N(c3cccc4ccccc34)c3cccc4oc5c(-c6ccccc6-c6ccccc6)cc6ccccc6c5c34)cc21 ; CC1(C)c2ccccc2-c2c(N(c3ccc(-c4ccccc4)cc3)c3cccc4oc5c(-c6ccccc6-c6ccccc6)cc6ccccc6c5c34)cccc21 ; c1ccc(-c2ccccc2-c2cc3ccccc3c3c2oc2cccc(N(c4ccccc4)c4ccc5c(c4)C(c4ccccc4)(c4ccccc4)c4ccccc4-5)c23)cc1 ; c1ccc(-c2ccccc2-c2cc3ccccc3c3c2oc2cccc(N(c4ccccc4)c4ccc5c(c4)C4(c6ccccc6-c6ccccc64)c4ccccc4-5)c23)cc1]

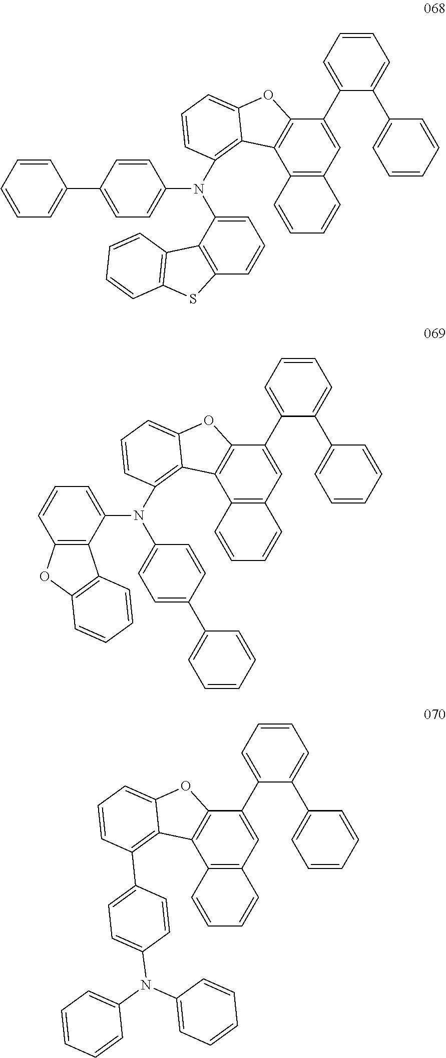

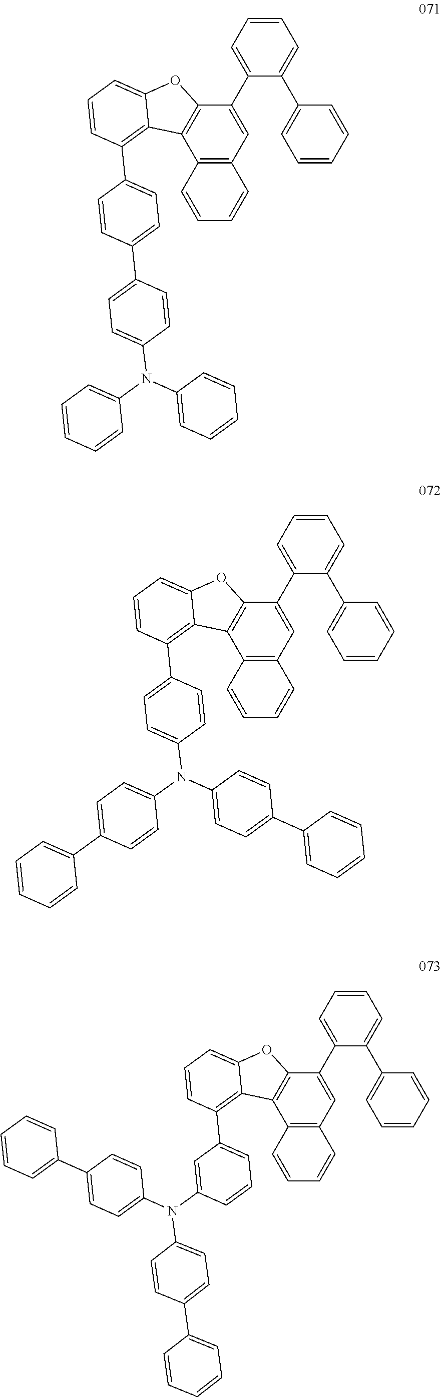 [SMILES: c1ccc(-c2ccccc2-c2cc3ccccc3c3c2oc2cccc(-c4ccc(-c5ccc(N(c6ccccc6)c6ccccc6)cc5)cc4)c23)cc1 ; c1ccc(-c2ccc(N(c3ccc(-c4ccccc4)cc3)c3ccc(-c4cccc5oc6c(-c7ccccc7-c7ccccc7)cc7ccccc7c6c45)cc3)cc2)cc1 ; c1ccc(-c2ccc(N(c3ccc(-c4ccccc4)cc3)c3cccc(-c4cccc5oc6c(-c7ccccc7-c7ccccc7)cc7ccccc7c6c45)c3)cc2)cc1]

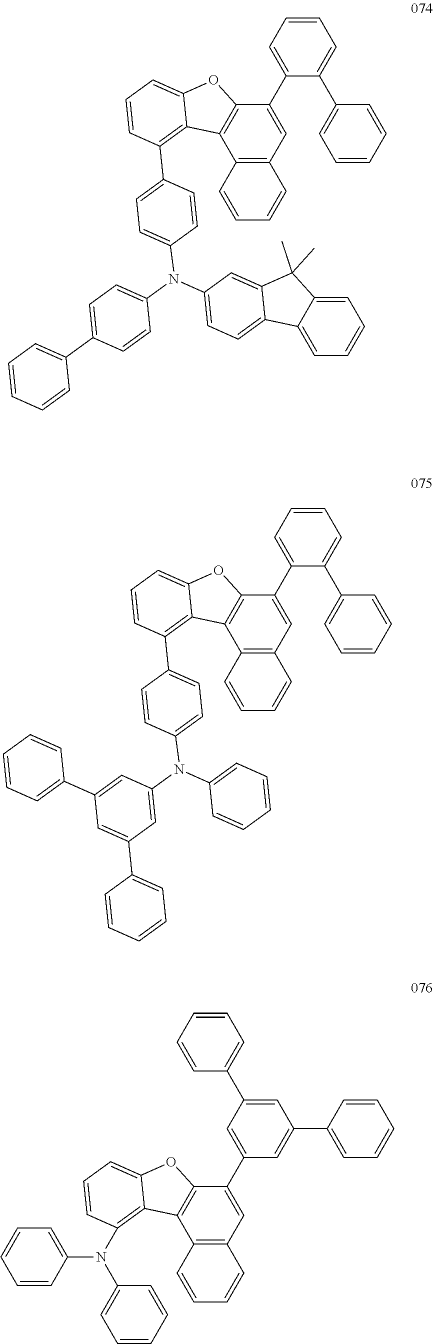 [SMILES: CC1(C)c2ccccc2-c2ccc(N(c3ccc(-c4ccccc4)cc3)c3ccc(-c4cccc5oc6c(-c7ccccc7-c7ccccc7)cc7ccccc7c6c45)cc3)cc21 ; c1ccc(-c2cc(-c3ccccc3)cc(N(c3ccccc3)c3ccc(-c4cccc5oc6c(-c7ccccc7-c7ccccc7)cc7ccccc7c6c45)cc3)c2)cc1 ; c1ccc(-c2cc(-c3ccccc3)cc(-c3cc4ccccc4c4c3oc3cccc(N(c5ccccc5)c5ccccc5)c34)c2)cc1]

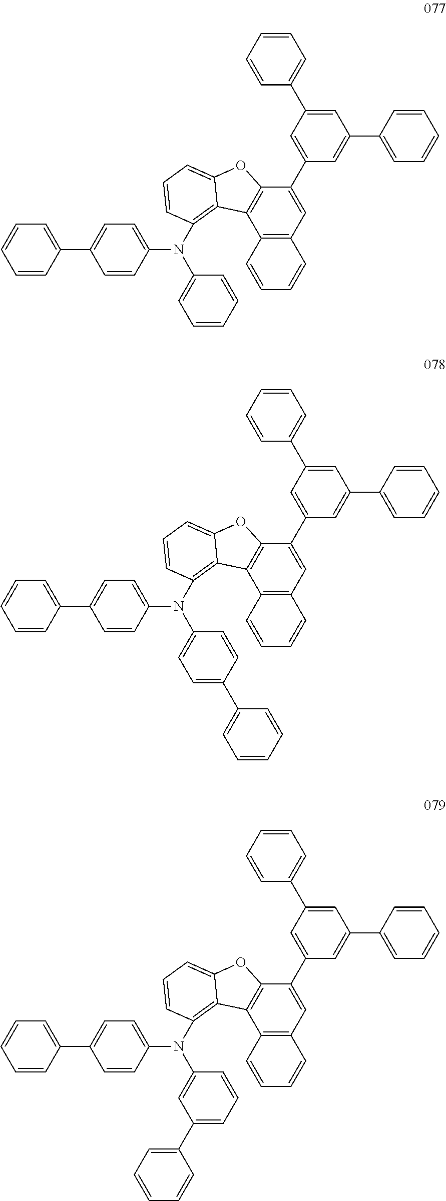 [SMILES: c1ccc(-c2ccc(N(c3ccccc3)c3cccc4oc5c(-c6cc(-c7ccccc7)cc(-c7ccccc7)c6)cc6ccccc6c5c34)cc2)cc1 ; c1ccc(-c2ccc(N(c3ccc(-c4ccccc4)cc3)c3cccc4oc5c(-c6cc(-c7ccccc7)cc(-c7ccccc7)c6)cc6ccccc6c5c34)cc2)cc1 ; c1ccc(-c2ccc(N(c3cccc(-c4ccccc4)c3)c3cccc4oc5c(-c6cc(-c7ccccc7)cc(-c7ccccc7)c6)cc6ccccc6c5c34)cc2)cc1]

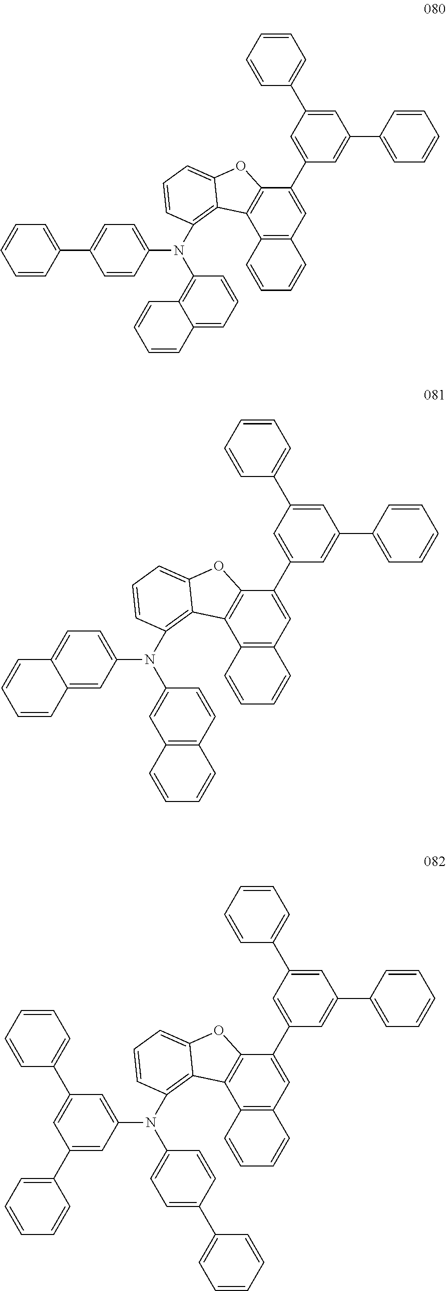 [SMILES: c1ccc(-c2ccc(N(c3cccc4ccccc34)c3cccc4oc5c(-c6cc(-c7ccccc7)cc(-c7ccccc7)c6)cc6ccccc6c5c34)cc2)cc1 ; c1ccc(-c2cc(-c3ccccc3)cc(-c3cc4ccccc4c4c3oc3cccc(N(c5ccc6ccccc6c5)c5ccc6ccccc6c5)c34)c2)cc1 ; c1ccc(-c2ccc(N(c3cc(-c4ccccc4)cc(-c4ccccc4)c3)c3cccc4oc5c(-c6cc(-c7ccccc7)cc(-c7ccccc7)c6)cc6ccccc6c5c34)cc2)cc1]

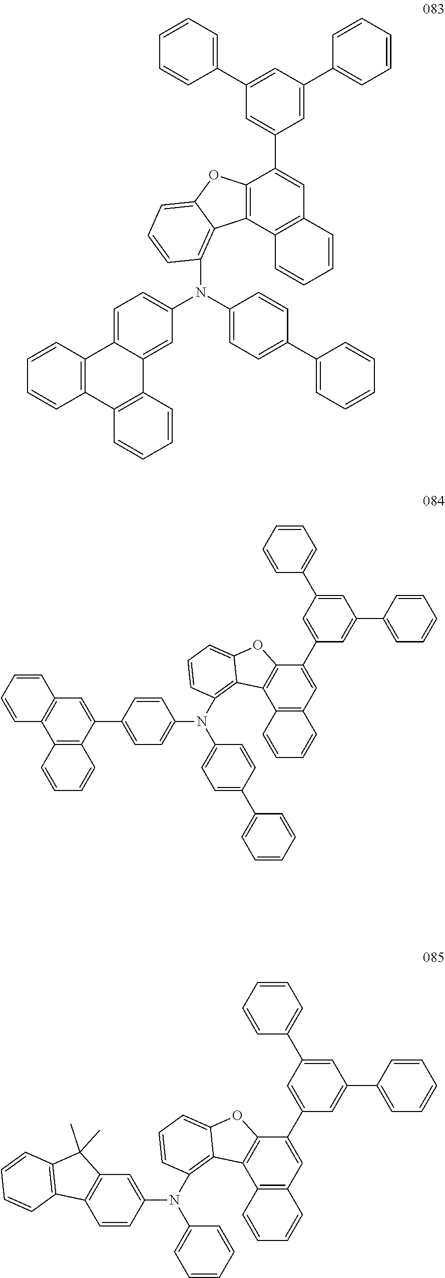 [SMILES: c1ccc(-c2ccc(N(c3ccc4c5ccccc5c5ccccc5c4c3)c3cccc4oc5c(-c6cc(-c7ccccc7)cc(-c7ccccc7)c6)cc6ccccc6c5c34)cc2)cc1 ; c1ccc(-c2ccc(N(c3ccc(-c4cc5ccccc5c5ccccc45)cc3)c3cccc4oc5c(-c6cc(-c7ccccc7)cc(-c7ccccc7)c6)cc6ccccc6c5c34)cc2)cc1 ; CC1(C)c2ccccc2-c2ccc(N(c3ccccc3)c3cccc4oc5c(-c6cc(-c7ccccc7)cc(-c7ccccc7)c6)cc6ccccc6c5c34)cc21]

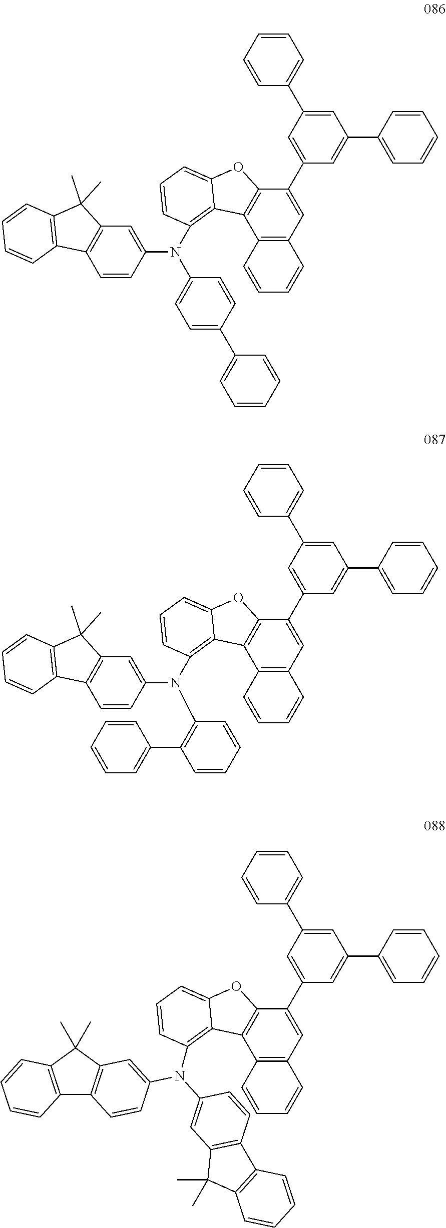 [SMILES: CC1(C)c2ccccc2-c2ccc(N(c3ccc(-c4ccccc4)cc3)c3cccc4oc5c(-c6cc(-c7ccccc7)cc(-c7ccccc7)c6)cc6ccccc6c5c34)cc21 ; CC1(C)c2ccccc2-c2ccc(N(c3ccccc3-c3ccccc3)c3cccc4oc5c(-c6cc(-c7ccccc7)cc(-c7ccccc7)c6)cc6ccccc6c5c34)cc21 ; CC1(C)c2ccccc2-c2ccc(N(c3ccc4c(c3)C(C)(C)c3ccccc3-4)c3cccc4oc5c(-c6cc(-c7ccccc7)cc(-c7ccccc7)c6)cc6ccccc6c5c34)cc21]

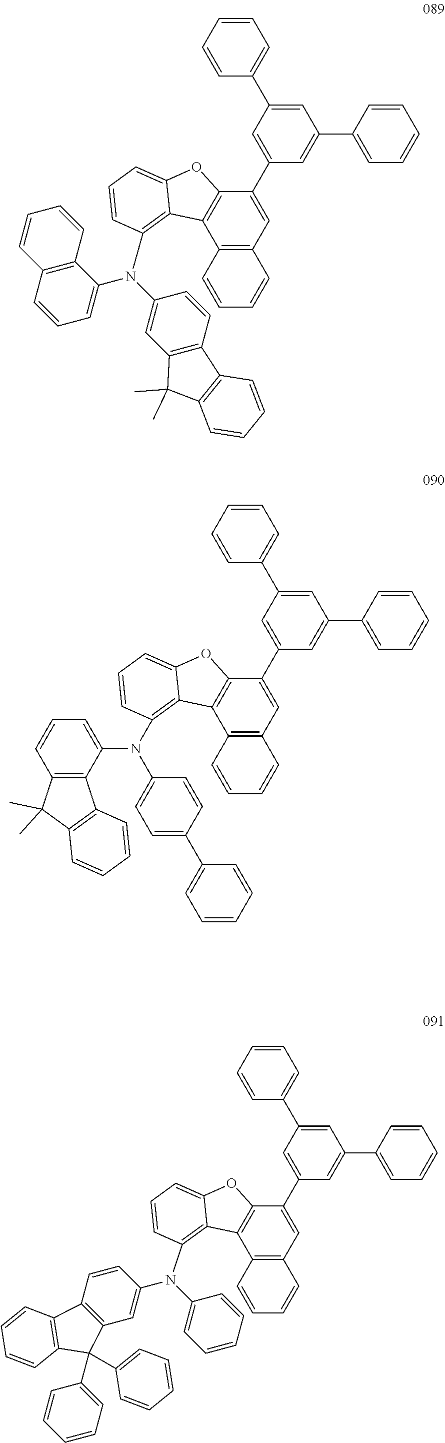 [SMILES: CC1(C)c2ccccc2-c2ccc(N(c3cccc4ccccc34)c3cccc4oc5c(-c6cc(-c7ccccc7)cc(-c7ccccc7)c6)cc6ccccc6c5c34)cc21 ; CC1(C)c2ccccc2-c2c(N(c3ccc(-c4ccccc4)cc3)c3cccc4oc5c(-c6cc(-c7ccccc7)cc(-c7ccccc7)c6)cc6ccccc6c5c34)cccc21 ; c1ccc(-c2cc(-c3ccccc3)cc(-c3cc4ccccc4c4c3oc3cccc(N(c5ccccc5)c5ccc6c(c5)C(c5ccccc5)(c5ccccc5)c5ccccc5-6)c34)c2)cc1]

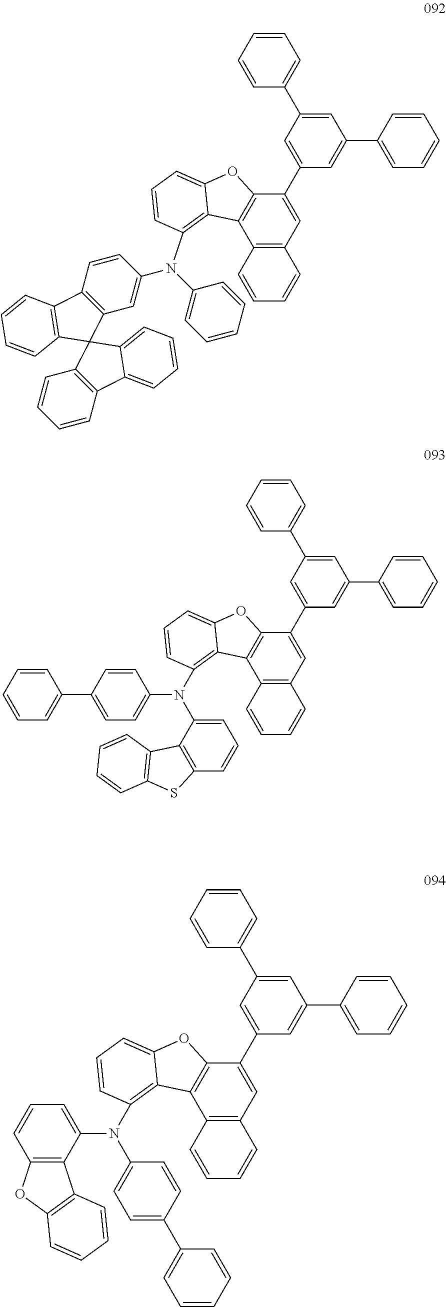 [SMILES: c1ccc(-c2cc(-c3ccccc3)cc(-c3cc4ccccc4c4c3oc3cccc(N(c5ccccc5)c5ccc6c(c5)C5(c7ccccc7-c7ccccc75)c5ccccc5-6)c34)c2)cc1 ; c1ccc(-c2ccc(N(c3cccc4sc5ccccc5c34)c3cccc4oc5c(-c6cc(-c7ccccc7)cc(-c7ccccc7)c6)cc6ccccc6c5c34)cc2)cc1 ; c1ccc(-c2ccc(N(c3cccc4oc5ccccc5c34)c3cccc4oc5c(-c6cc(-c7ccccc7)cc(-c7ccccc7)c6)cc6ccccc6c5c34)cc2)cc1]

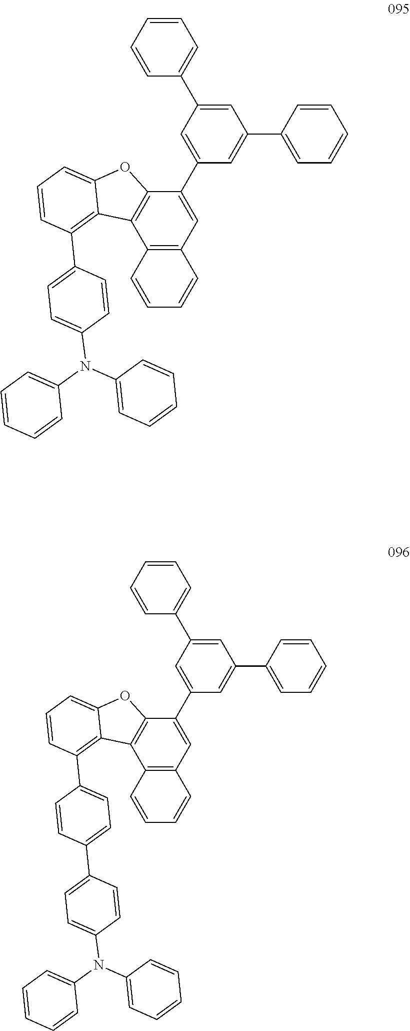 [SMILES: c1ccc(-c2cc(-c3ccccc3)cc(-c3cc4ccccc4c4c3oc3cccc(-c5ccc(N(c6ccccc6)c6ccccc6)cc5)c34)c2)cc1 ; c1ccc(-c2cc(-c3ccccc3)cc(-c3cc4ccccc4c4c3oc3cccc(-c5ccc(-c6ccc(N(c7ccccc7)c7ccccc7)cc6)cc5)c34)c2)cc1]

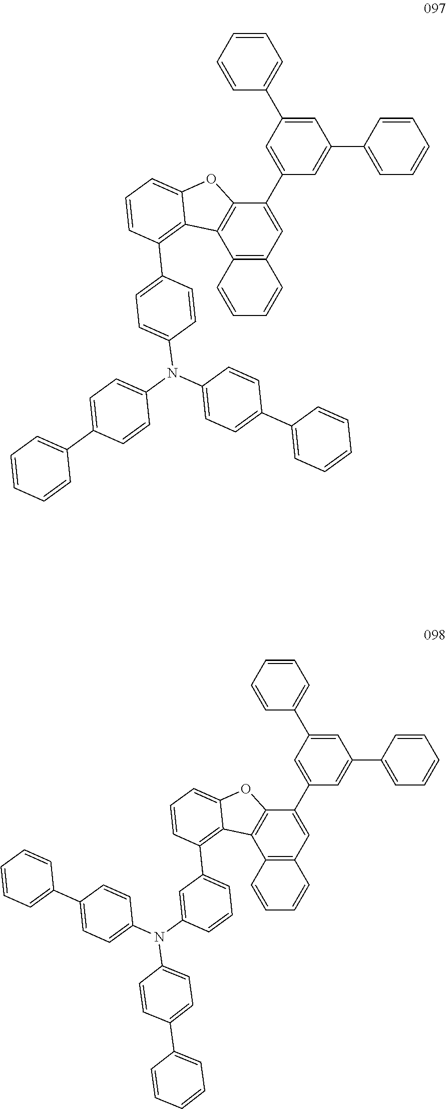 [SMILES: c1ccc(-c2ccc(N(c3ccc(-c4ccccc4)cc3)c3ccc(-c4cccc5oc6c(-c7cc(-c8ccccc8)cc(-c8ccccc8)c7)cc7ccccc7c6c45)cc3)cc2)cc1 ; c1ccc(-c2ccc(N(c3ccc(-c4ccccc4)cc3)c3cccc(-c4cccc5oc6c(-c7cc(-c8ccccc8)cc(-c8ccccc8)c7)cc7ccccc7c6c45)c3)cc2)cc1]

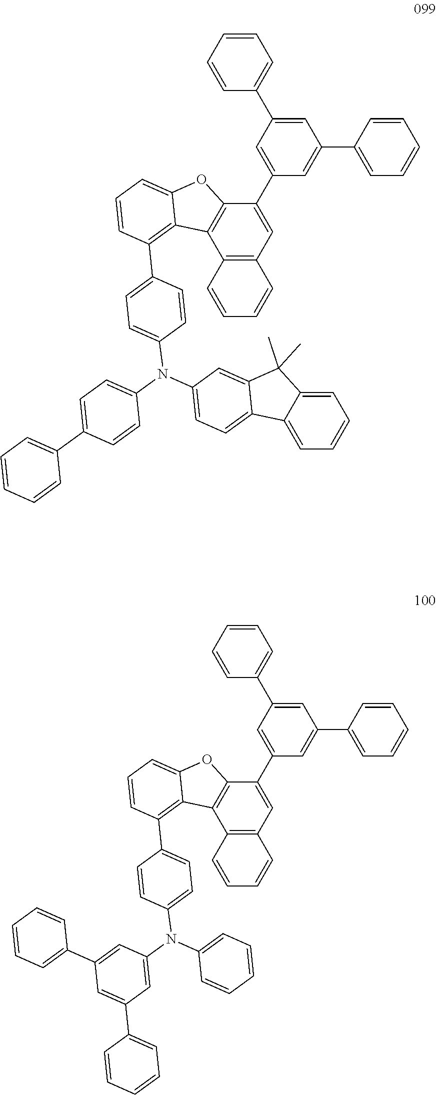 [SMILES: CC1(C)c2ccccc2-c2ccc(N(c3ccc(-c4ccccc4)cc3)c3ccc(-c4cccc5oc6c(-c7cc(-c8ccccc8)cc(-c8ccccc8)c7)cc7ccccc7c6c45)cc3)cc21 ; c1ccc(-c2cc(-c3ccccc3)cc(-c3cc4ccccc4c4c3oc3cccc(-c5ccc(N(c6ccccc6)c6cc(-c7ccccc7)cc(-c7ccccc7)c6)cc5)c34)c2)cc1]

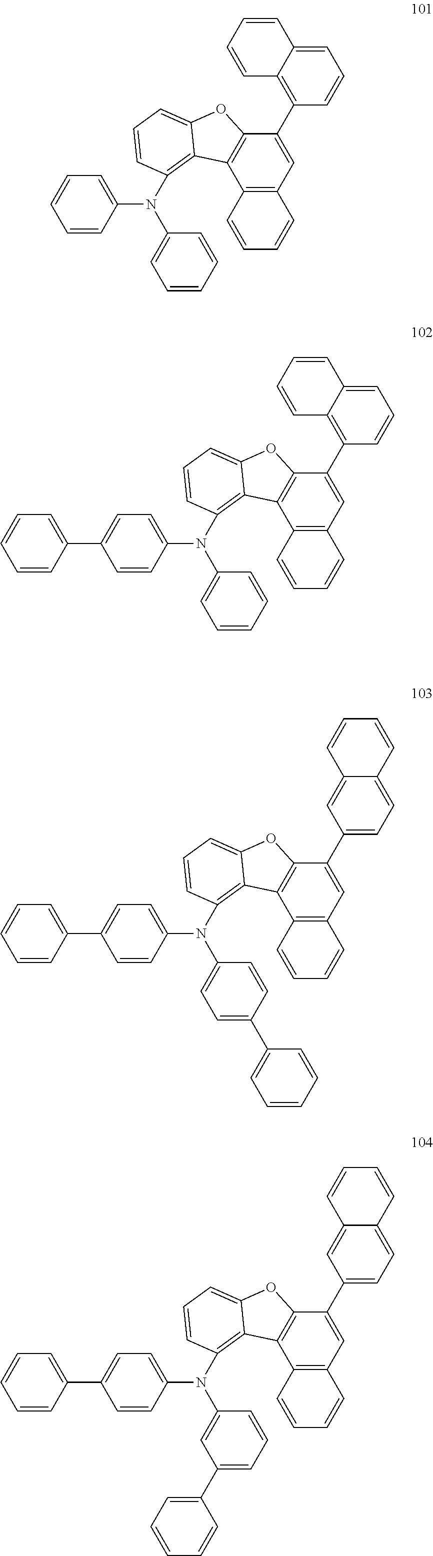 [SMILES: c1ccc(N(c2ccccc2)c2cccc3oc4c(-c5cccc6ccccc56)cc5ccccc5c4c23)cc1 ; c1ccc(-c2ccc(N(c3ccccc3)c3cccc4oc5c(-c6cccc7ccccc67)cc6ccccc6c5c34)cc2)cc1 ; c1ccc(-c2ccc(N(c3ccc(-c4ccccc4)cc3)c3cccc4oc5c(-c6ccc7ccccc7c6)cc6ccccc6c5c34)cc2)cc1 ; c1ccc(-c2ccc(N(c3cccc(-c4ccccc4)c3)c3cccc4oc5c(-c6ccc7ccccc7c6)cc6ccccc6c5c34)cc2)cc1]

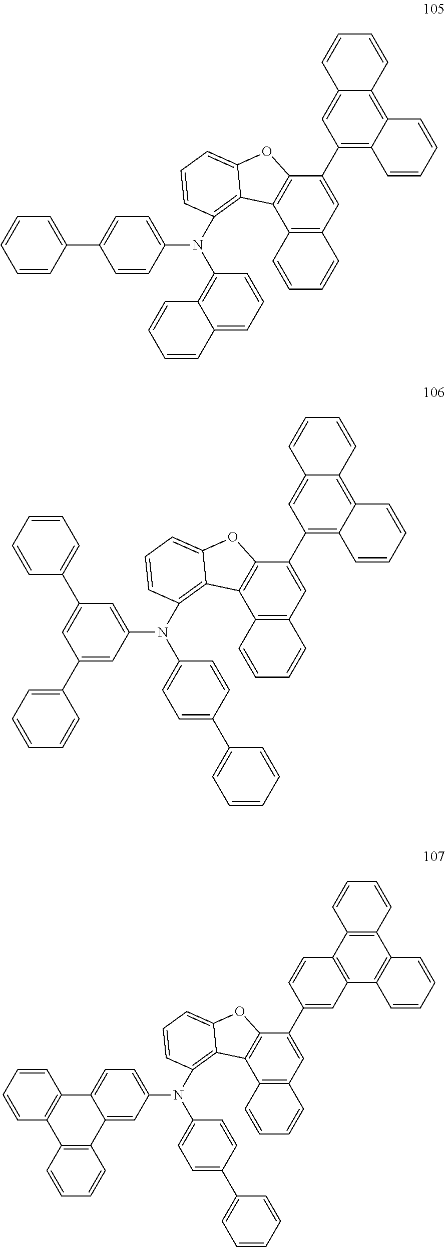 [SMILES: c1ccc(-c2ccc(N(c3cccc4ccccc34)c3cccc4oc5c(-c6cc7ccccc7c7ccccc67)cc6ccccc6c5c34)cc2)cc1 ; c1ccc(-c2ccc(N(c3cc(-c4ccccc4)cc(-c4ccccc4)c3)c3cccc4oc5c(-c6cc7ccccc7c7ccccc67)cc6ccccc6c5c34)cc2)cc1 ; c1ccc(-c2ccc(N(c3ccc4c5ccccc5c5ccccc5c4c3)c3cccc4oc5c(-c6ccc7c8ccccc8c8ccccc8c7c6)cc6ccccc6c5c34)cc2)cc1]

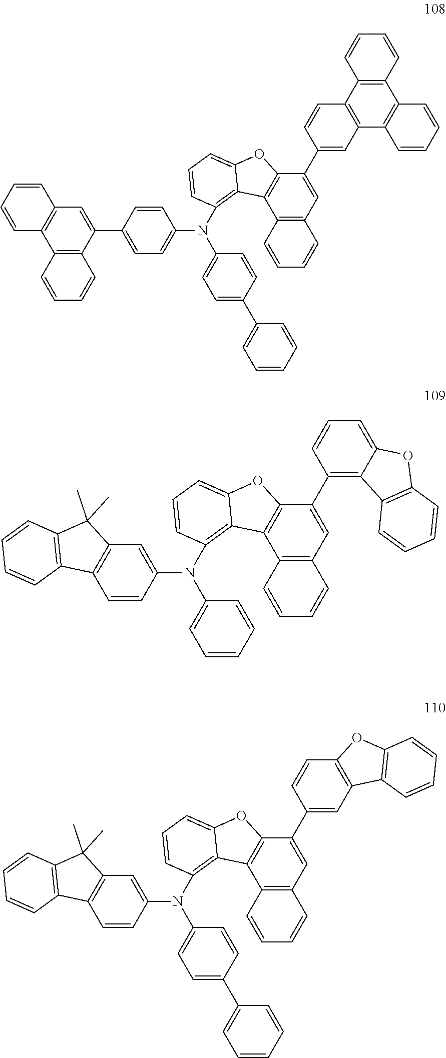 [SMILES: c1ccc(-c2ccc(N(c3ccc(-c4cc5ccccc5c5ccccc45)cc3)c3cccc4oc5c(-c6ccc7c8ccccc8c8ccccc8c7c6)cc6ccccc6c5c34)cc2)cc1 ; CC1(C)c2ccccc2-c2ccc(N(c3ccccc3)c3cccc4oc5c(-c6cccc7oc8ccccc8c67)cc6ccccc6c5c34)cc21 ; CC1(C)c2ccccc2-c2ccc(N(c3ccc(-c4ccccc4)cc3)c3cccc4oc5c(-c6ccc7oc8ccccc8c7c6)cc6ccccc6c5c34)cc21]

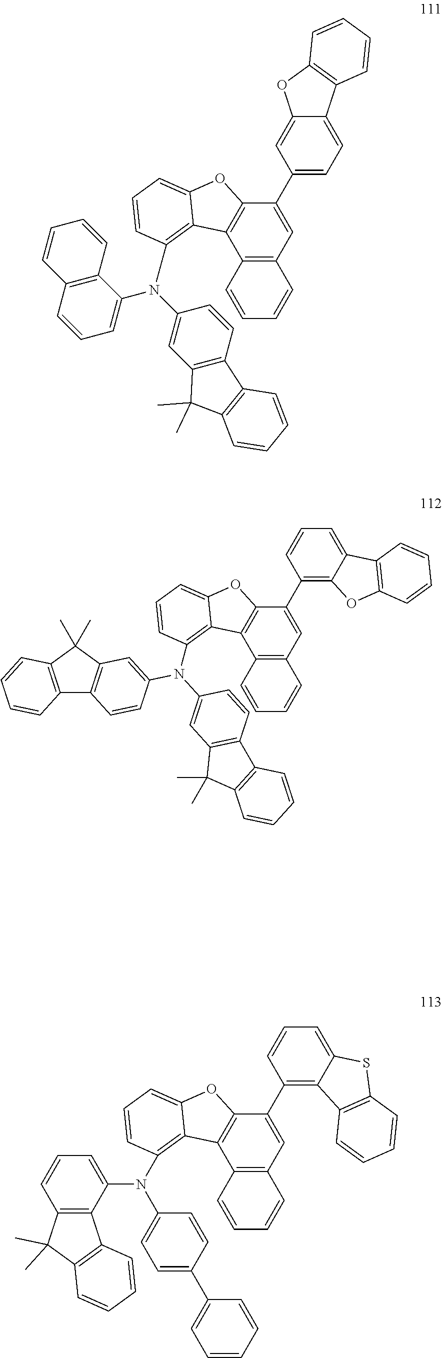 [SMILES: CC1(C)c2ccccc2-c2ccc(N(c3cccc4ccccc34)c3cccc4oc5c(-c6ccc7c(c6)oc6ccccc67)cc6ccccc6c5c34)cc21 ; CC1(C)c2ccccc2-c2ccc(N(c3ccc4c(c3)C(C)(C)c3ccccc3-4)c3cccc4oc5c(-c6cccc7c6oc6ccccc67)cc6ccccc6c5c34)cc21 ; CC1(C)c2ccccc2-c2c(N(c3ccc(-c4ccccc4)cc3)c3cccc4oc5c(-c6cccc7sc8ccccc8c67)cc6ccccc6c5c34)cccc21]

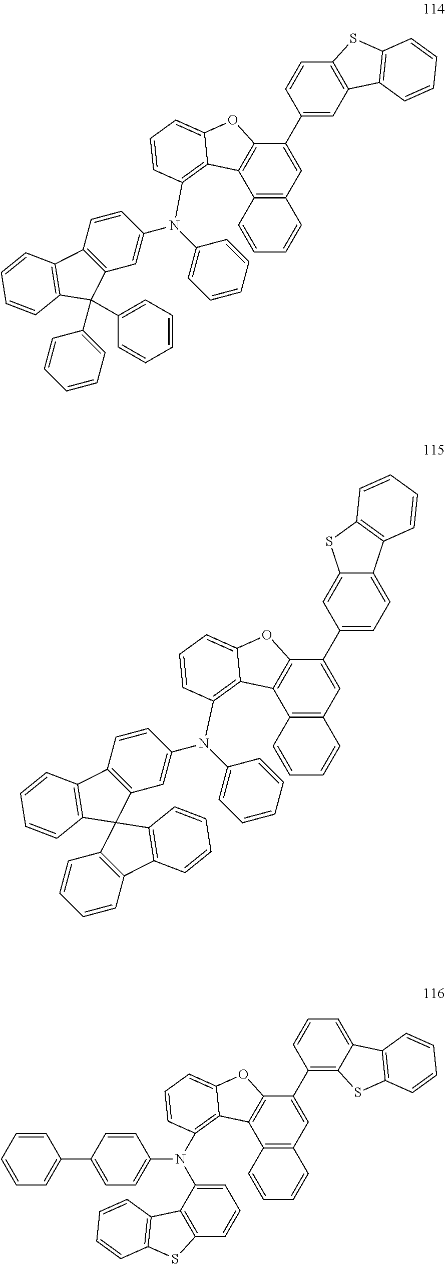 [SMILES: c1ccc(N(c2ccc3c(c2)C(c2ccccc2)(c2ccccc2)c2ccccc2-3)c2cccc3oc4c(-c5ccc6sc7ccccc7c6c5)cc5ccccc5c4c23)cc1 ; c1ccc(N(c2ccc3c(c2)C2(c4ccccc4-c4ccccc42)c2ccccc2-3)c2cccc3oc4c(-c5ccc6c(c5)sc5ccccc56)cc5ccccc5c4c23)cc1 ; c1ccc(-c2ccc(N(c3cccc4sc5ccccc5c34)c3cccc4oc5c(-c6cccc7c6sc6ccccc67)cc6ccccc6c5c34)cc2)cc1]

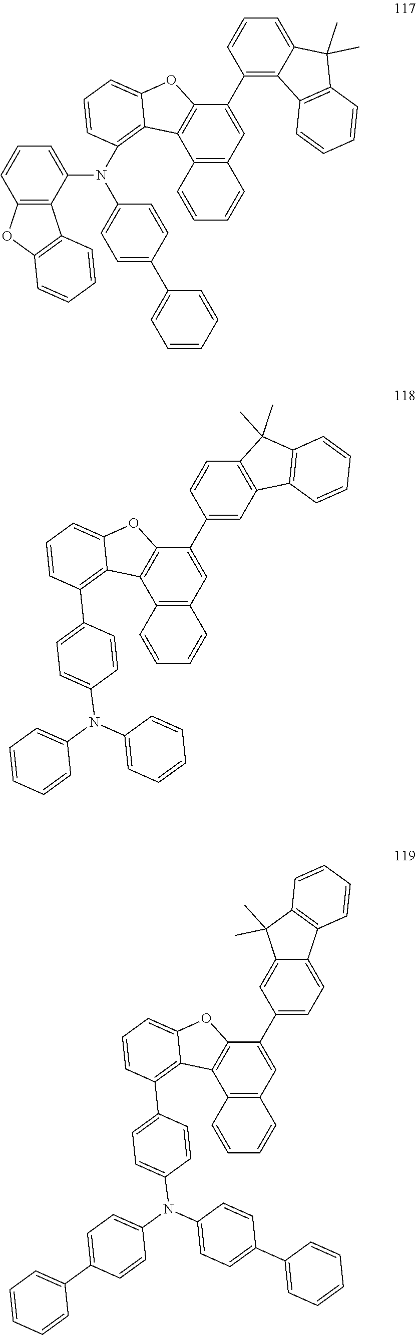 [SMILES: CC1(C)c2ccccc2-c2c(-c3cc4ccccc4c4c3oc3cccc(N(c5ccc(-c6ccccc6)cc5)c5cccc6oc7ccccc7c56)c34)cccc21 ; CC1(C)c2ccccc2-c2cc(-c3cc4ccccc4c4c3oc3cccc(-c5ccc(N(c6ccccc6)c6ccccc6)cc5)c34)ccc21 ; CC1(C)c2ccccc2-c2ccc(-c3cc4ccccc4c4c3oc3cccc(-c5ccc(N(c6ccc(-c7ccccc7)cc6)c6ccc(-c7ccccc7)cc6)cc5)c34)cc21]

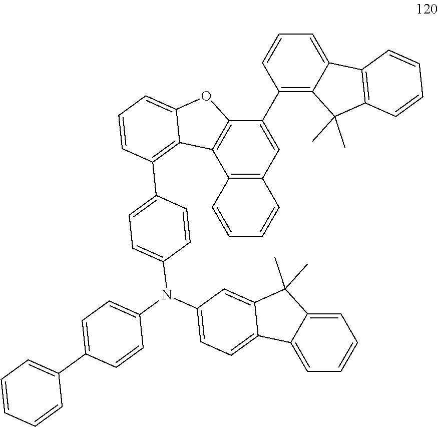 [SMILES: CC1(C)c2ccccc2-c2ccc(N(c3ccc(-c4ccccc4)cc3)c3ccc(-c4cccc5oc6c(-c7cccc8c7C(C)(C)c7ccccc7-8)cc7ccccc7c6c45)cc3)cc21]

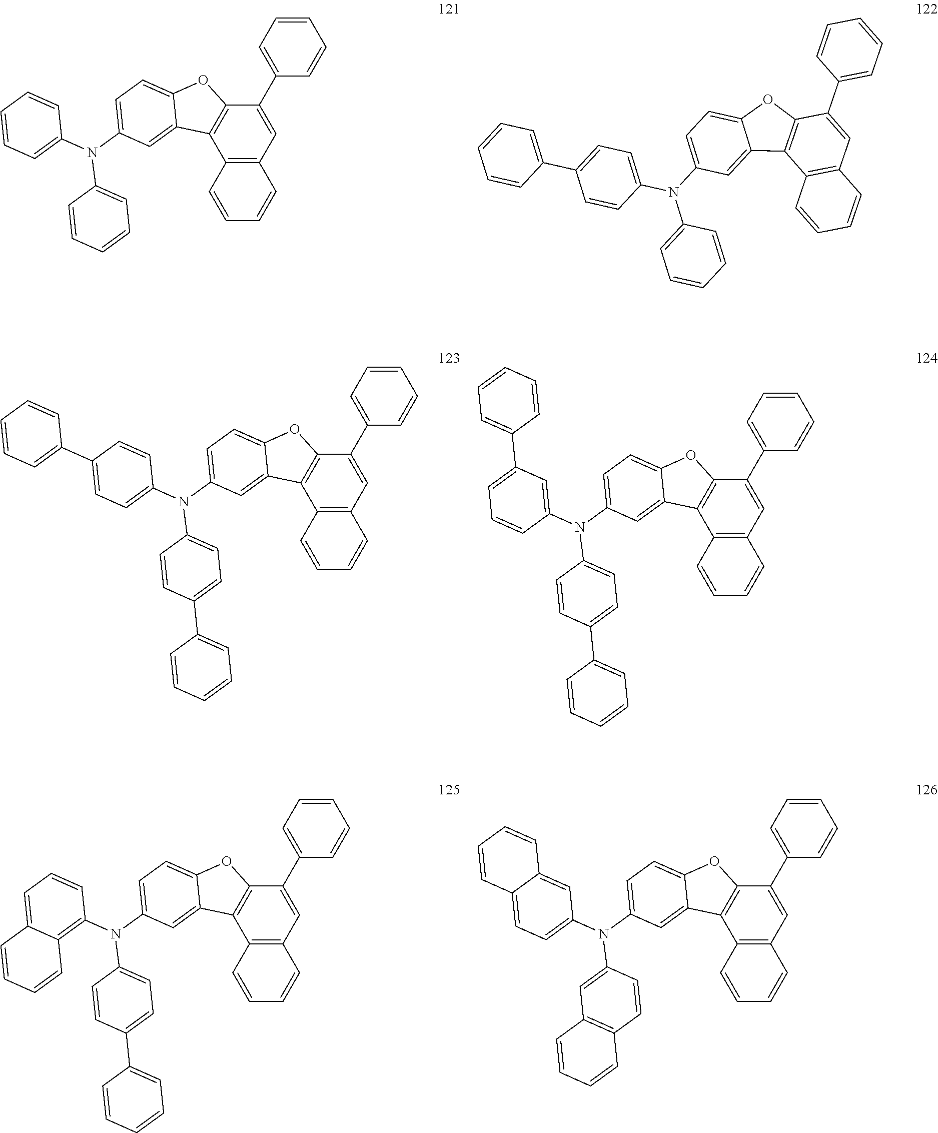 [SMILES: c1ccc(-c2cc3ccccc3c3c2oc2ccc(N(c4ccccc4)c4ccccc4)cc23)cc1 ; c1ccc(-c2ccc(N(c3ccccc3)c3ccc4oc5c(-c6ccccc6)cc6ccccc6c5c4c3)cc2)cc1 ; c1ccc(-c2ccc(N(c3ccc(-c4ccccc4)cc3)c3ccc4oc5c(-c6ccccc6)cc6ccccc6c5c4c3)cc2)cc1 ; c1ccc(-c2ccc(N(c3cccc(-c4ccccc4)c3)c3ccc4oc5c(-c6ccccc6)cc6ccccc6c5c4c3)cc2)cc1 ; c1ccc(-c2ccc(N(c3ccc4oc5c(-c6ccccc6)cc6ccccc6c5c4c3)c3cccc4ccccc34)cc2)cc1 ; c1ccc(-c2cc3ccccc3c3c2oc2ccc(N(c4ccc5ccccc5c4)c4ccc5ccccc5c4)cc23)cc1]

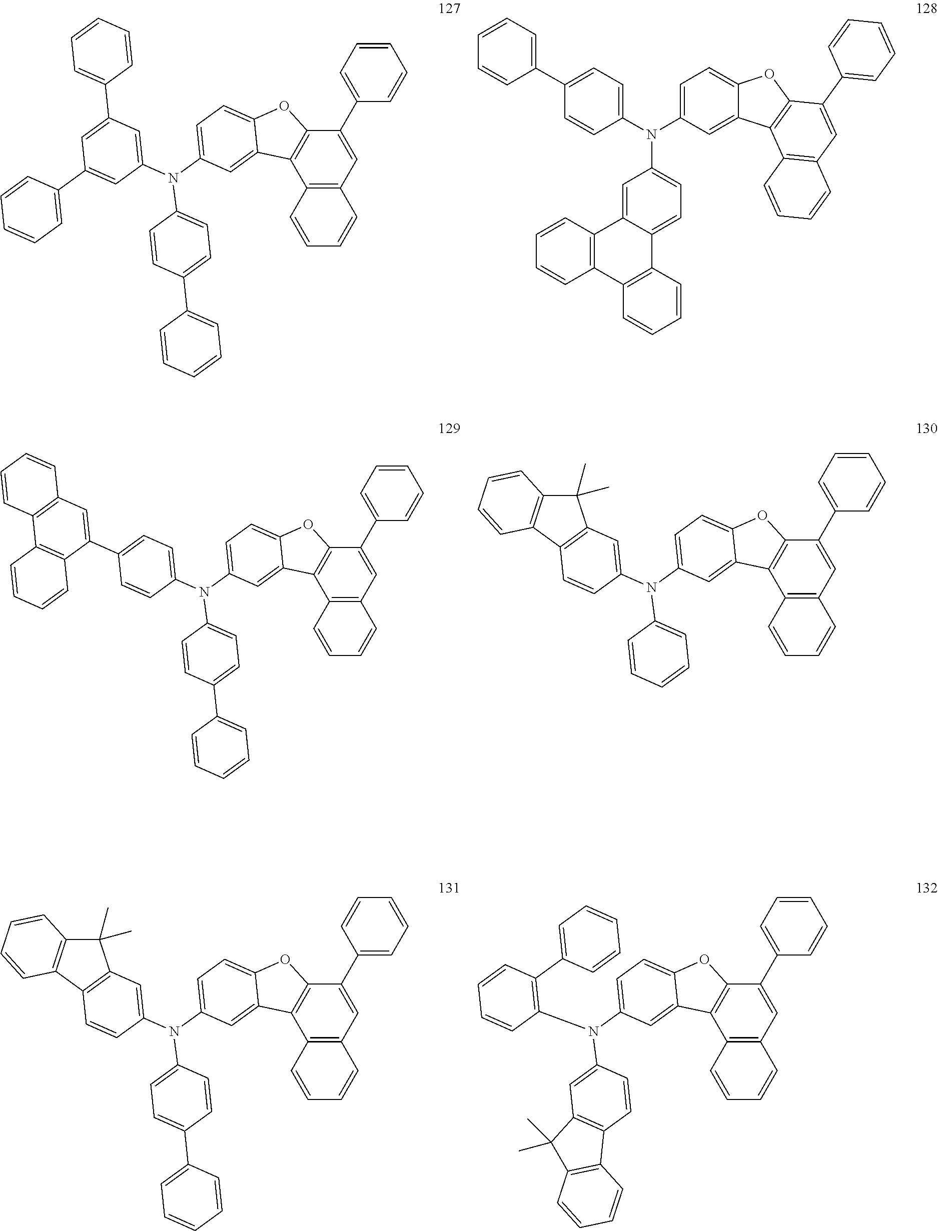

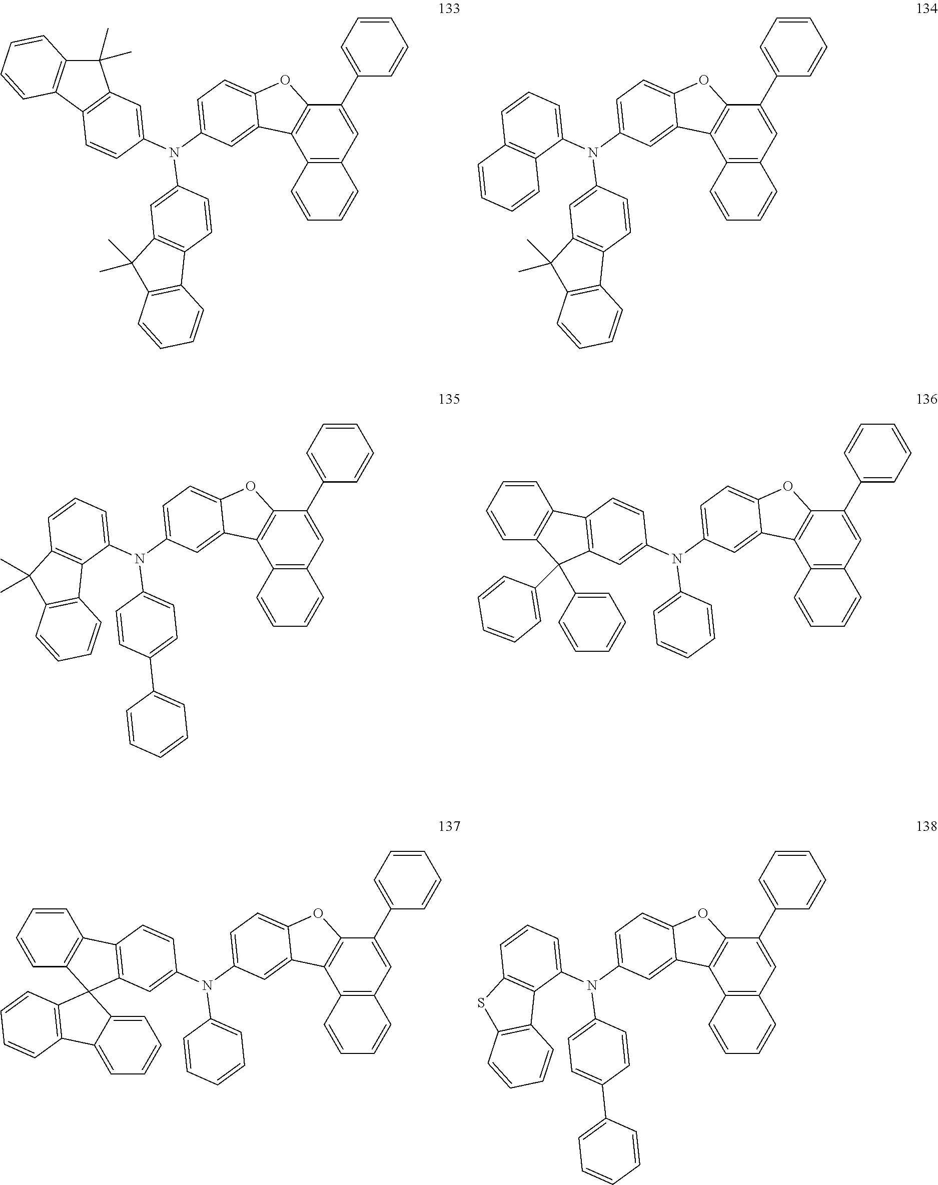

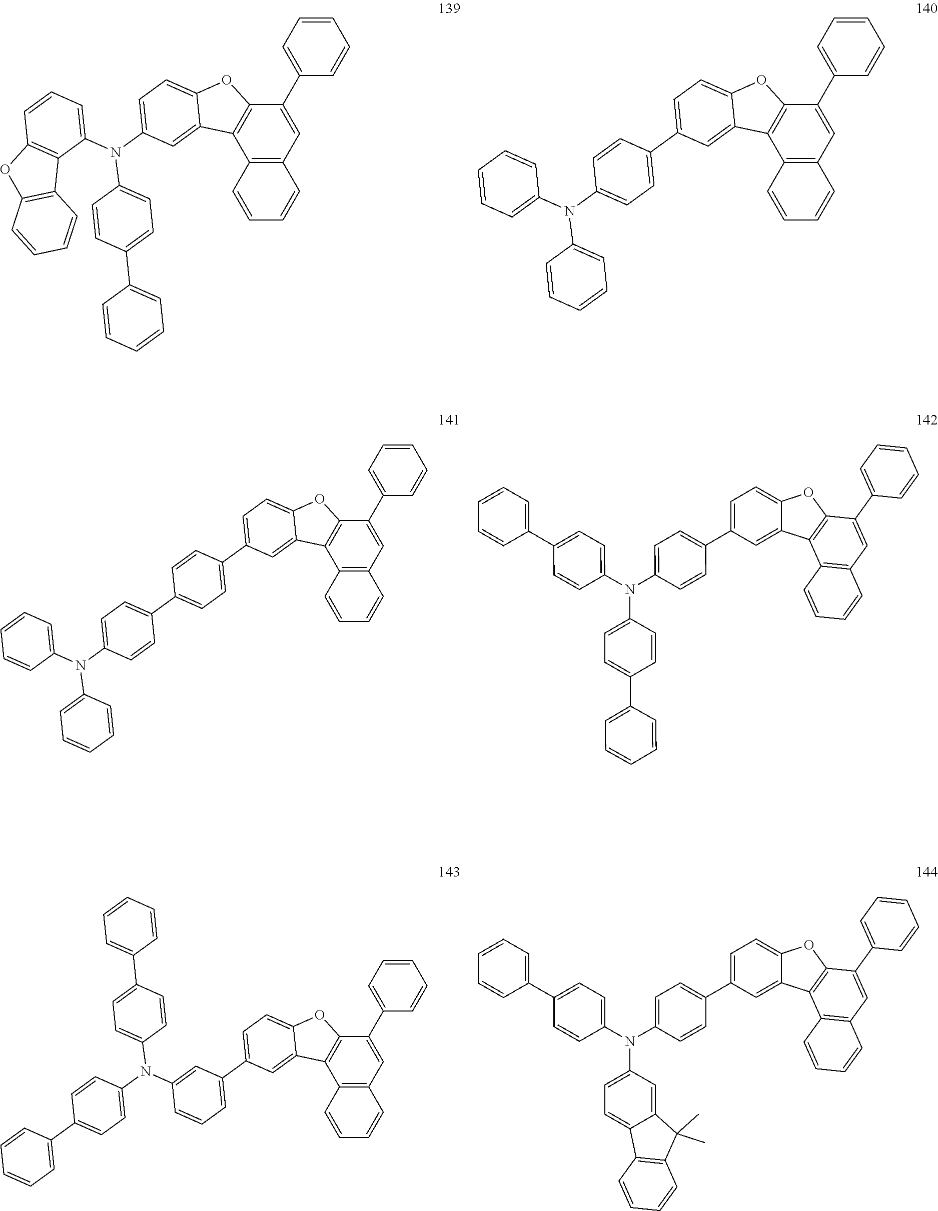

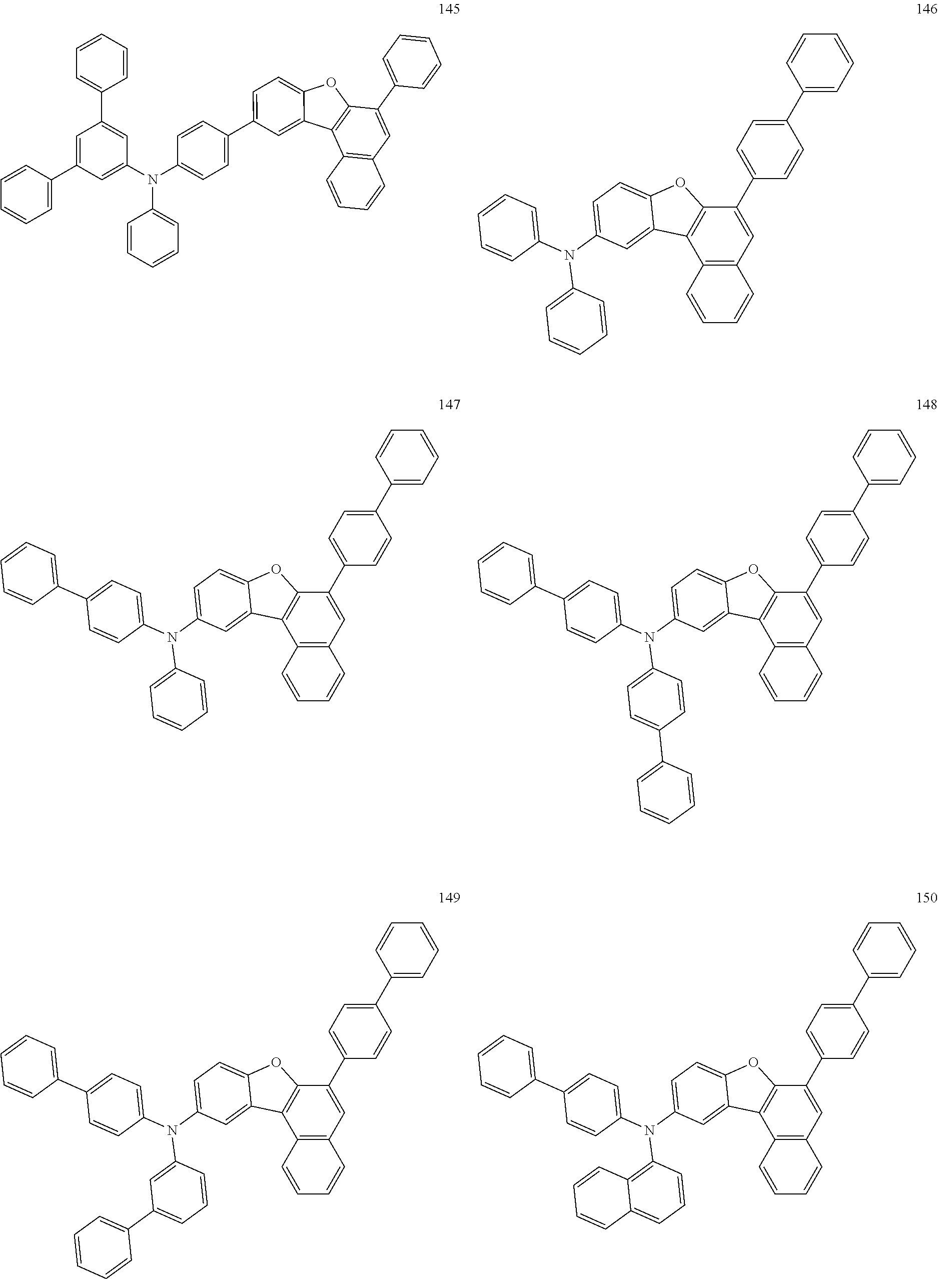

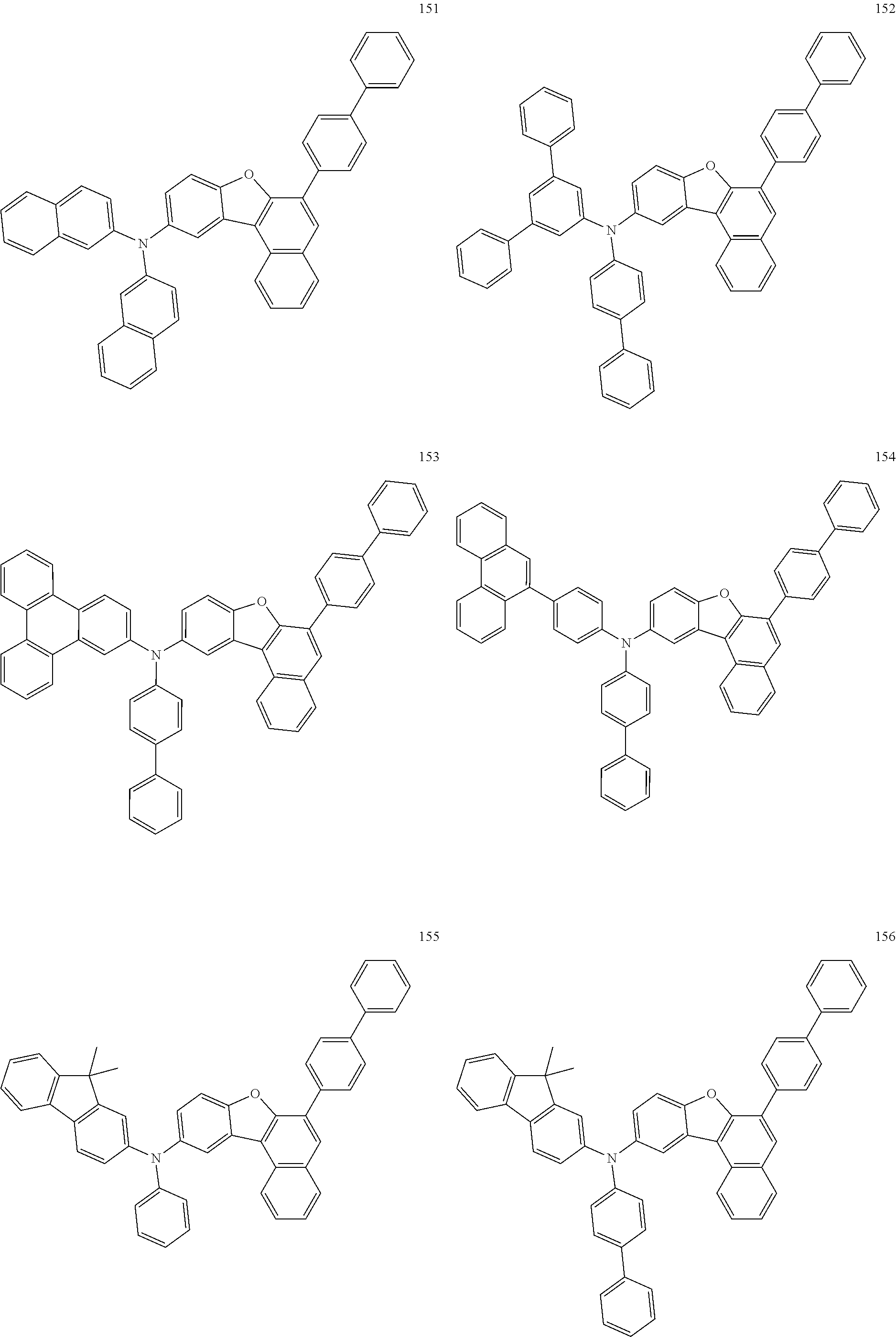

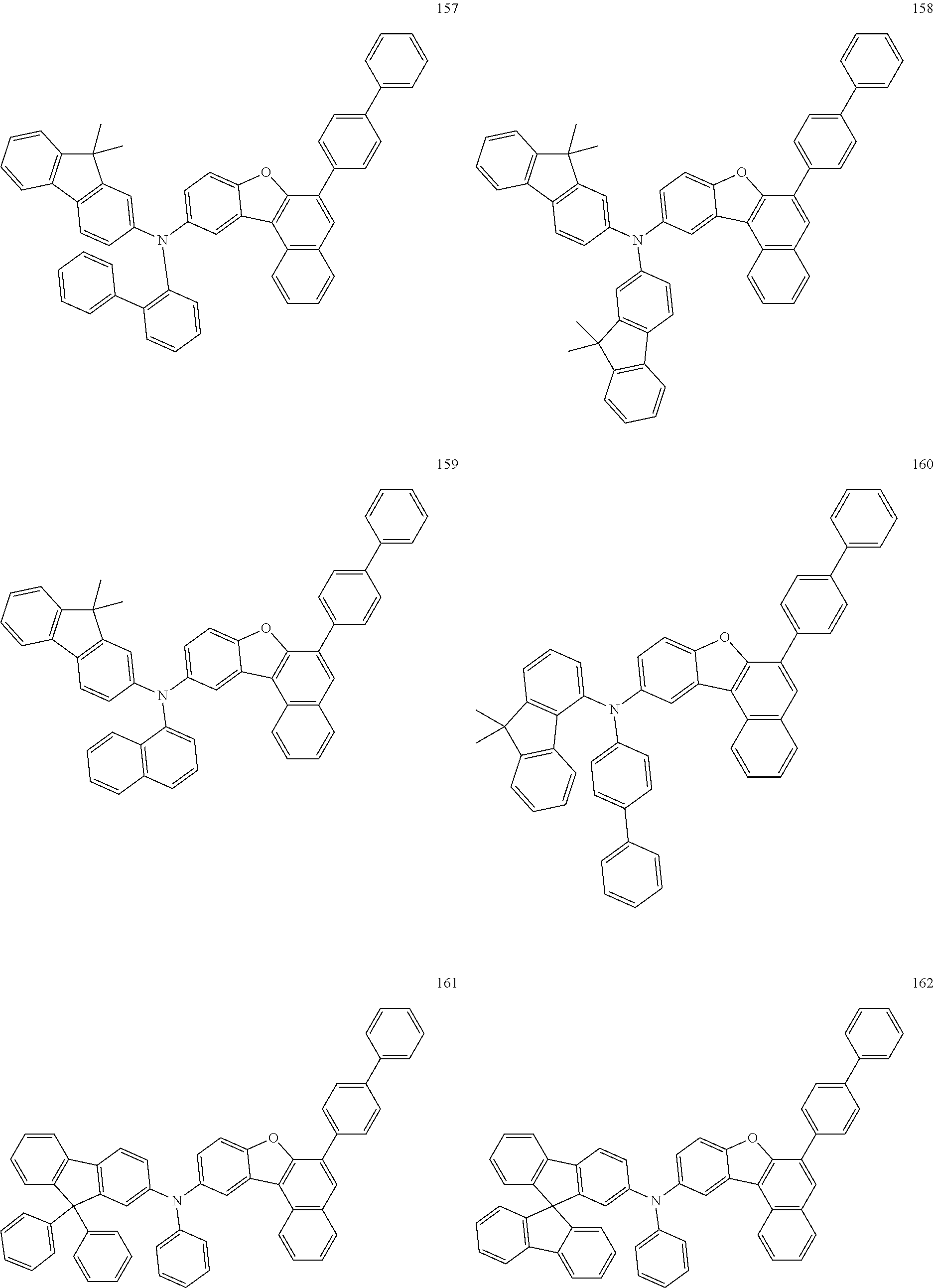

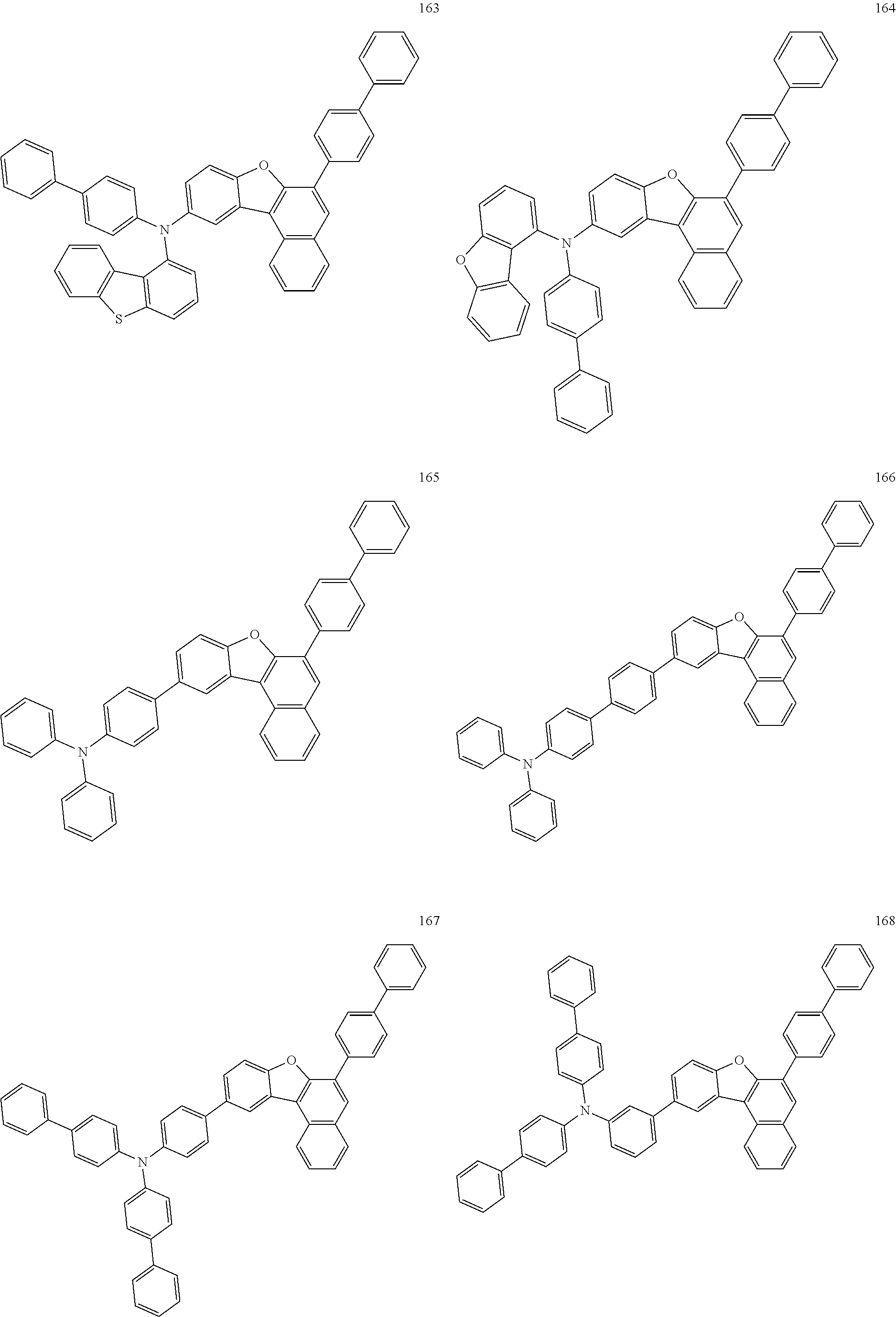

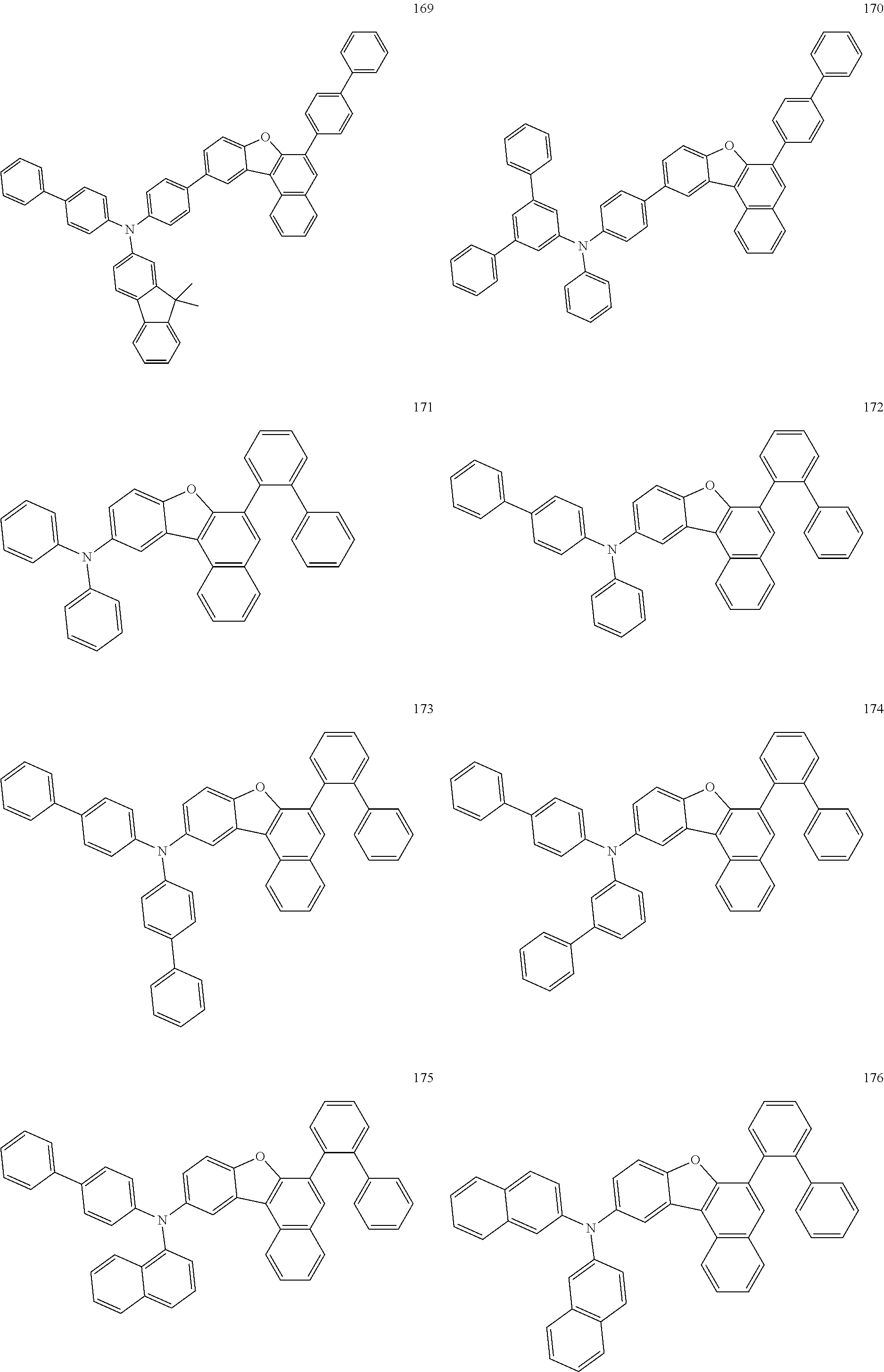

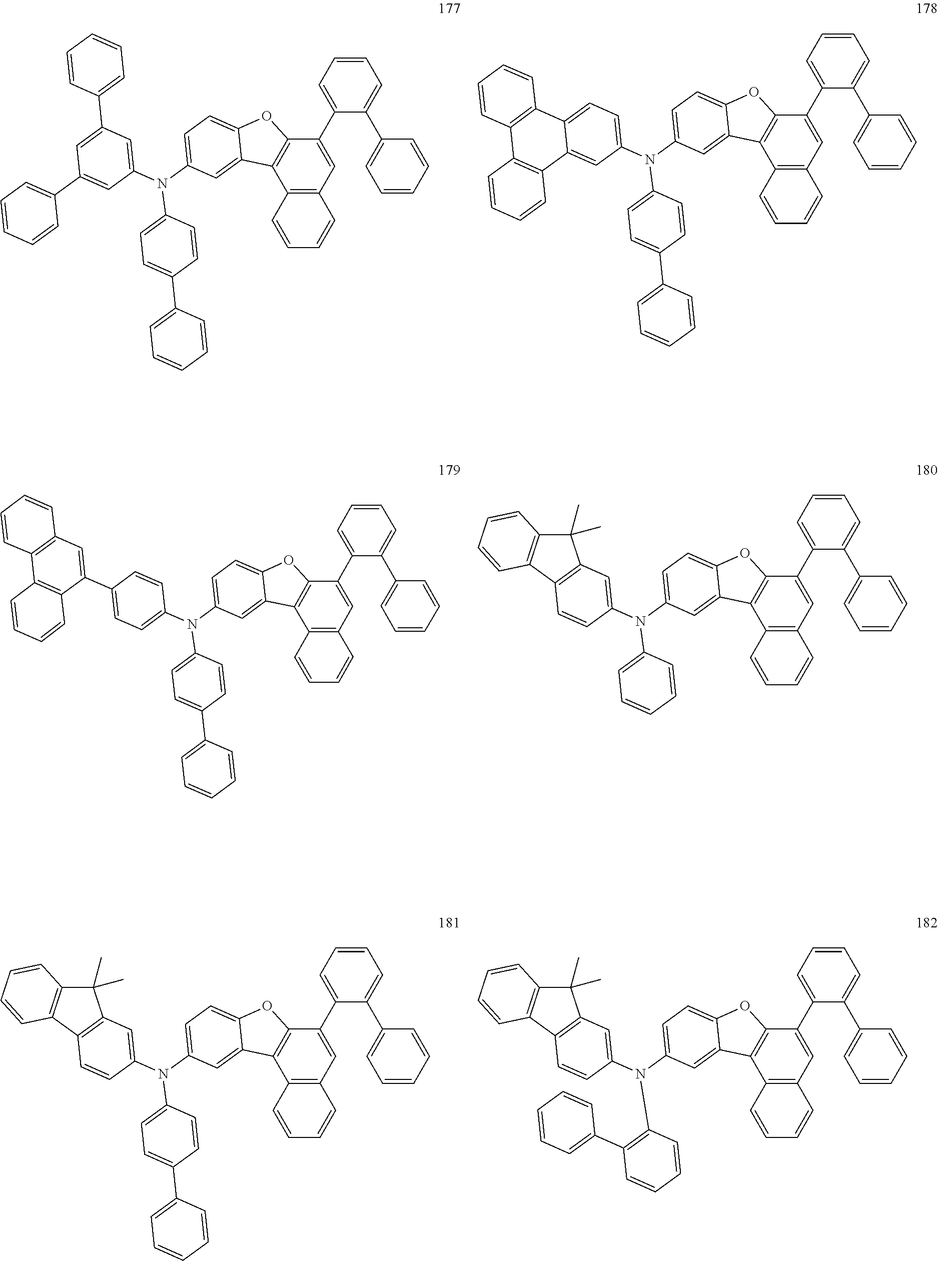

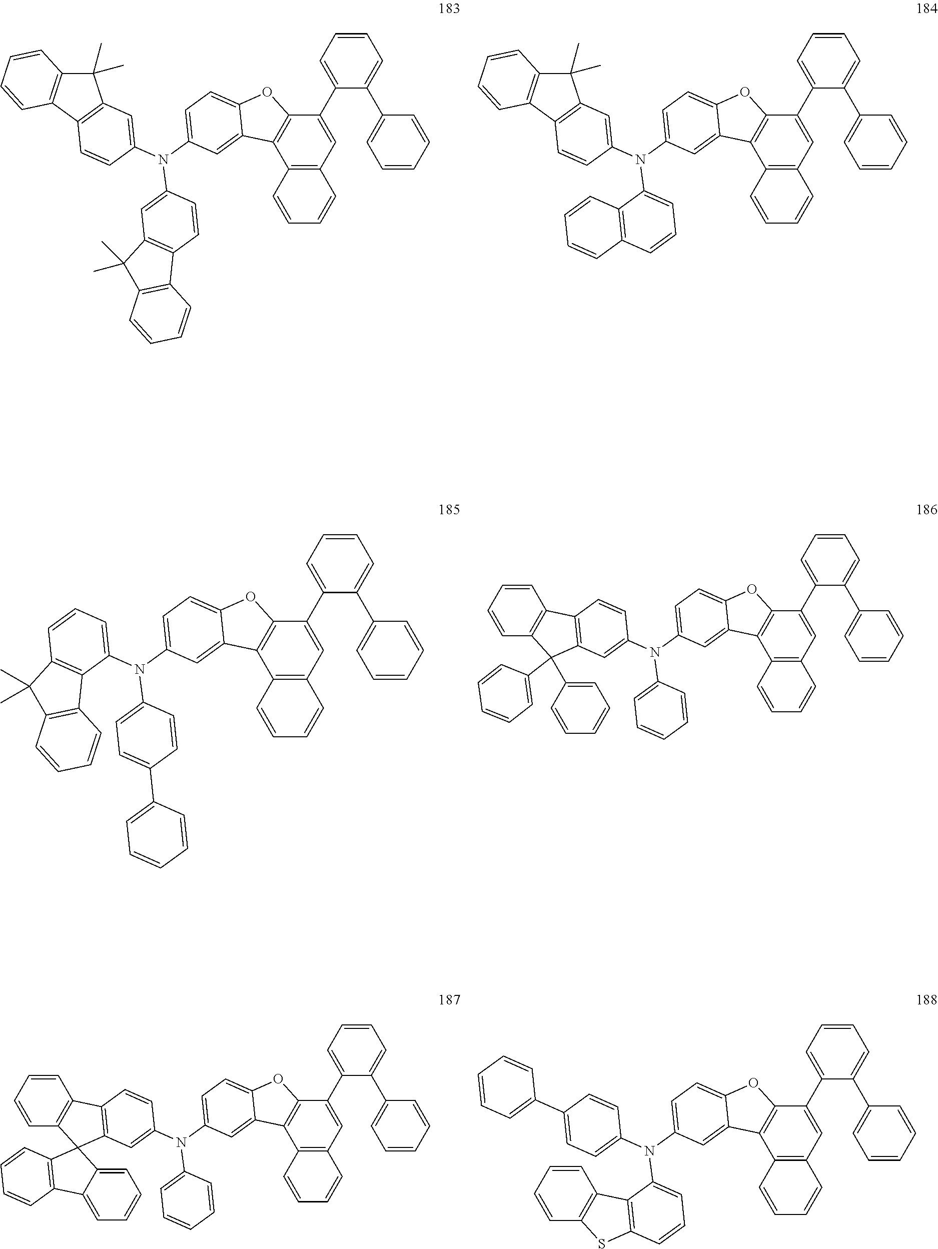

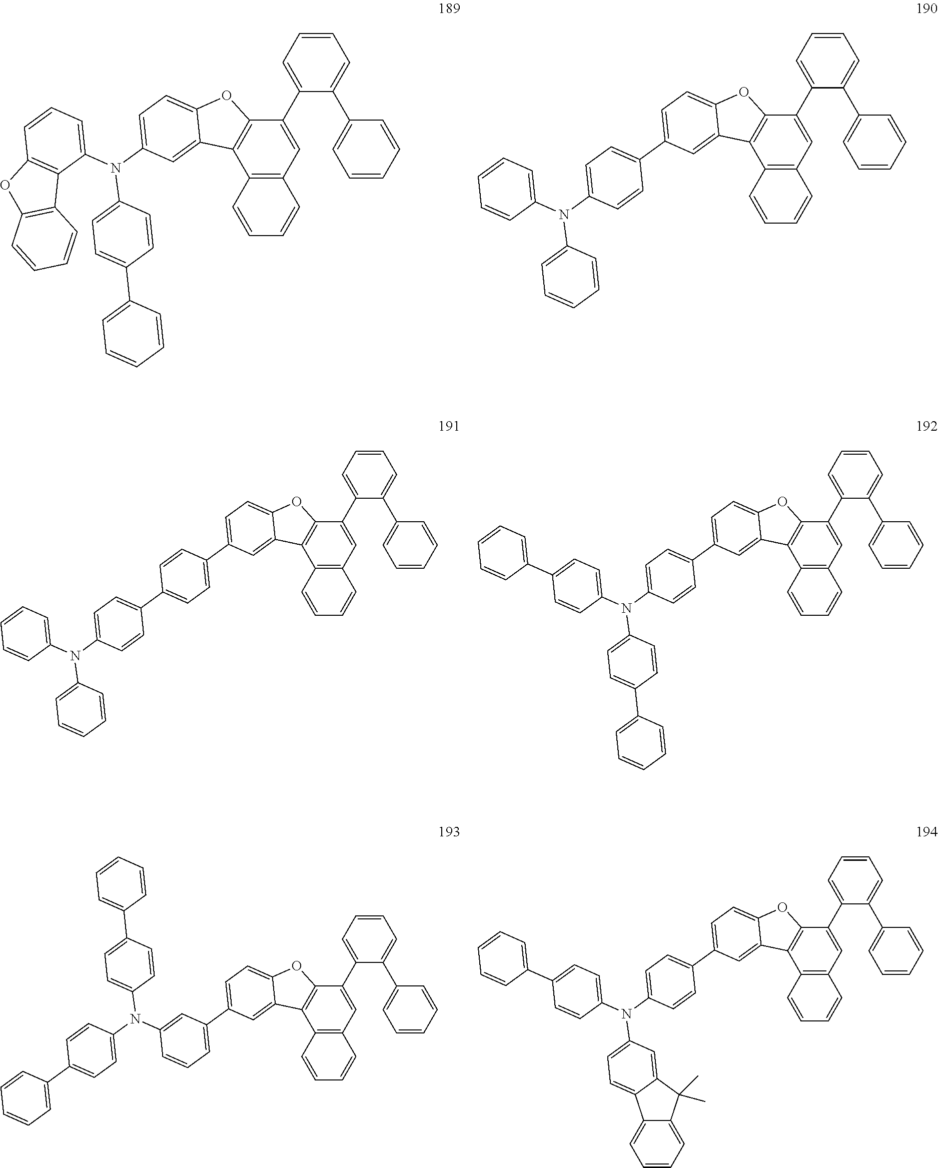

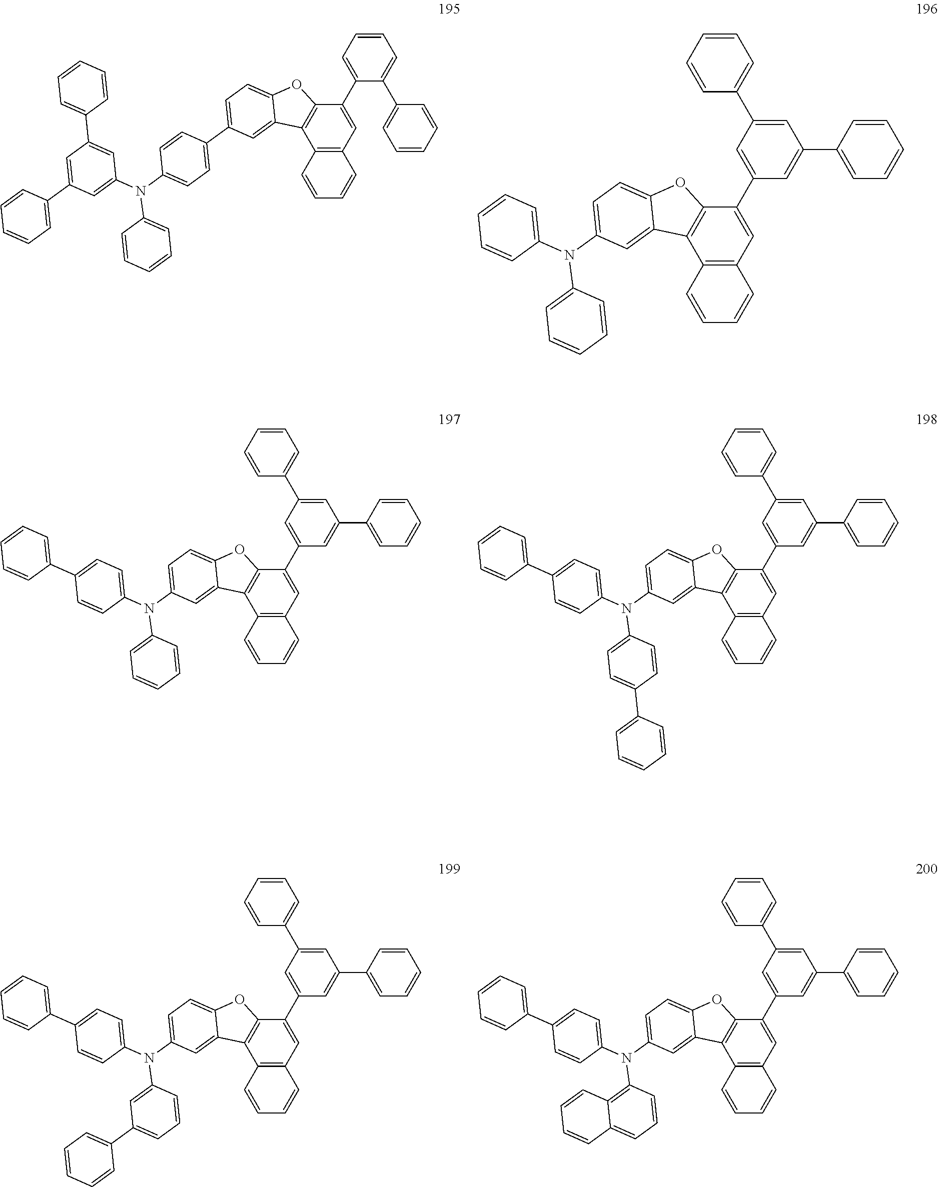

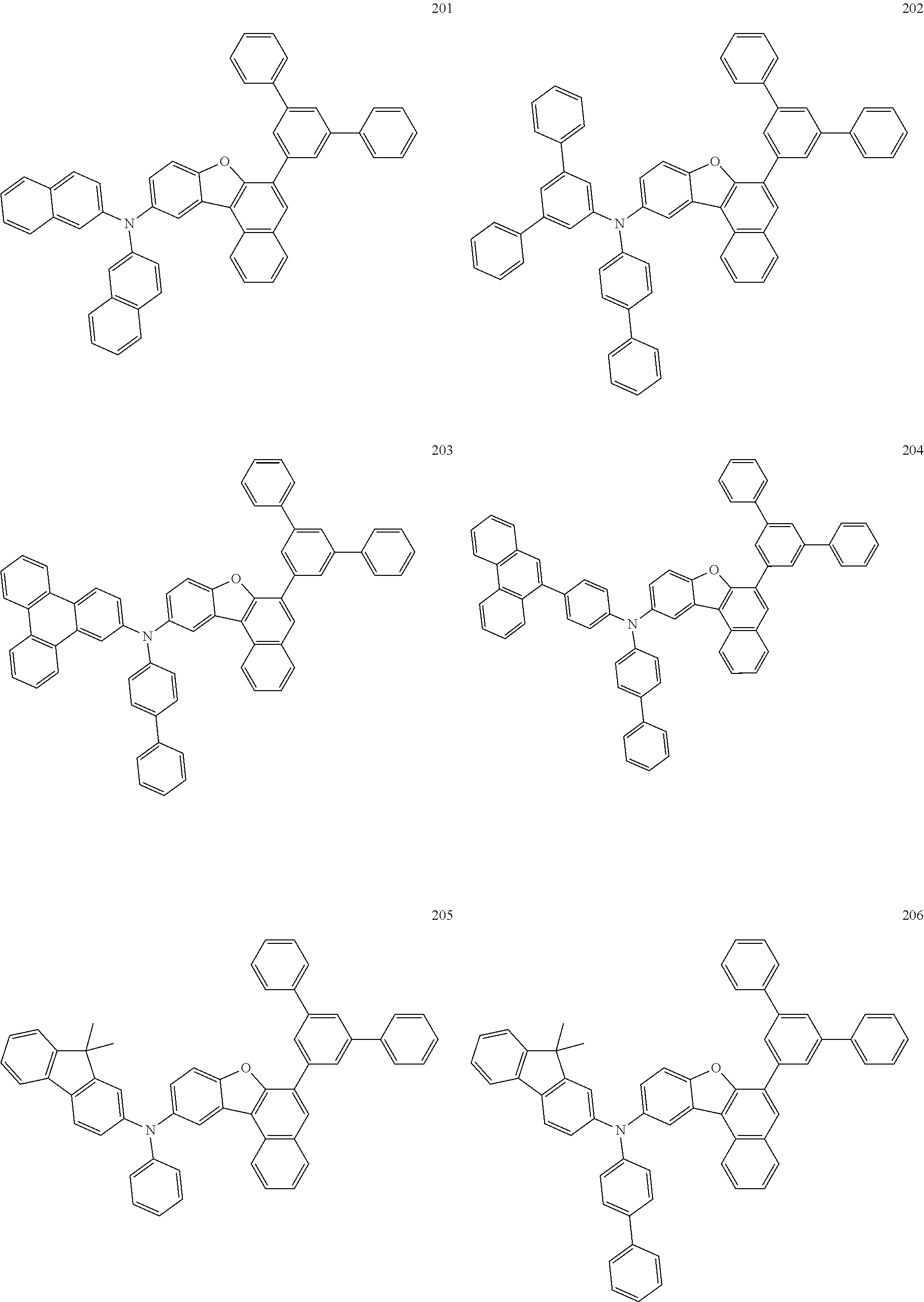

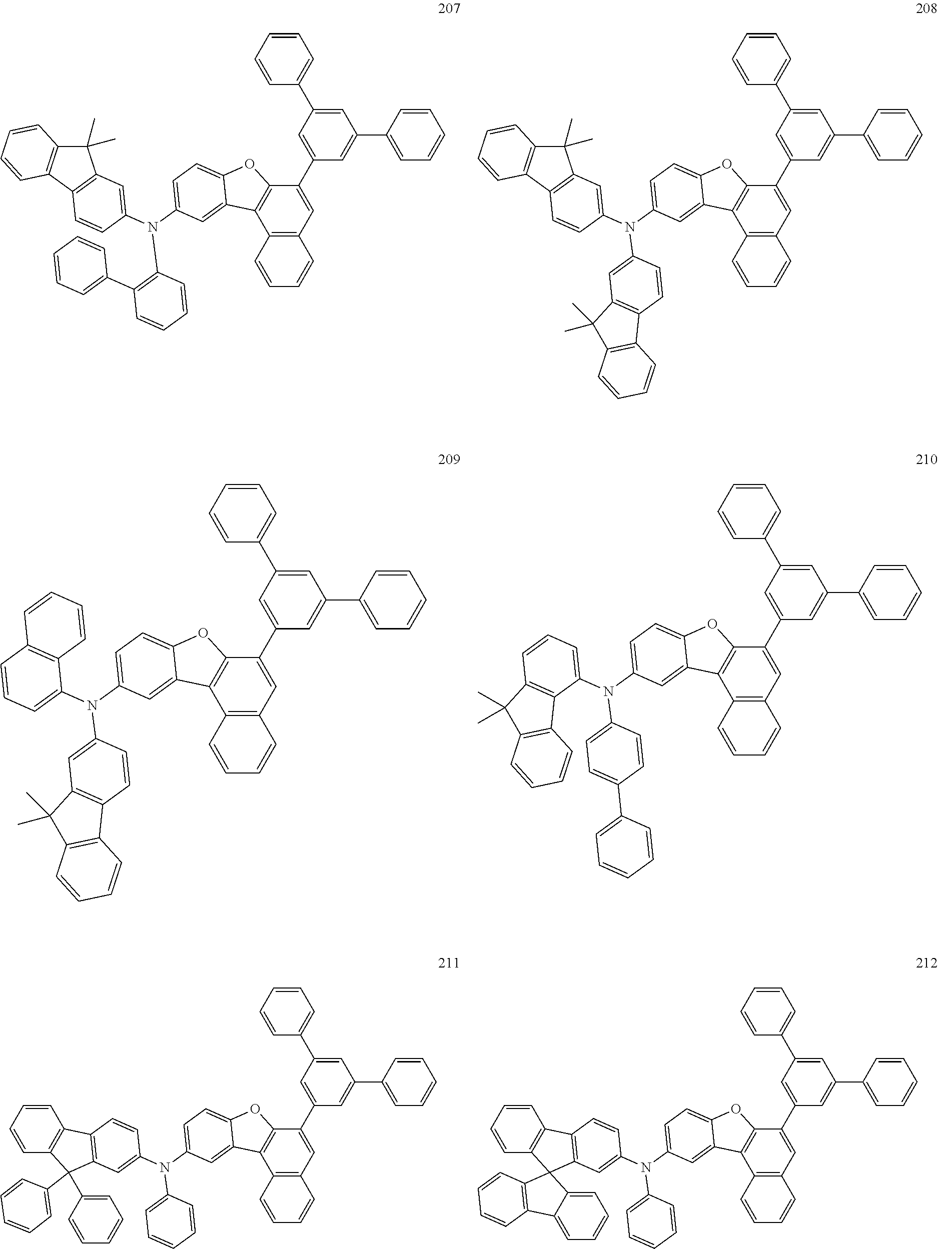

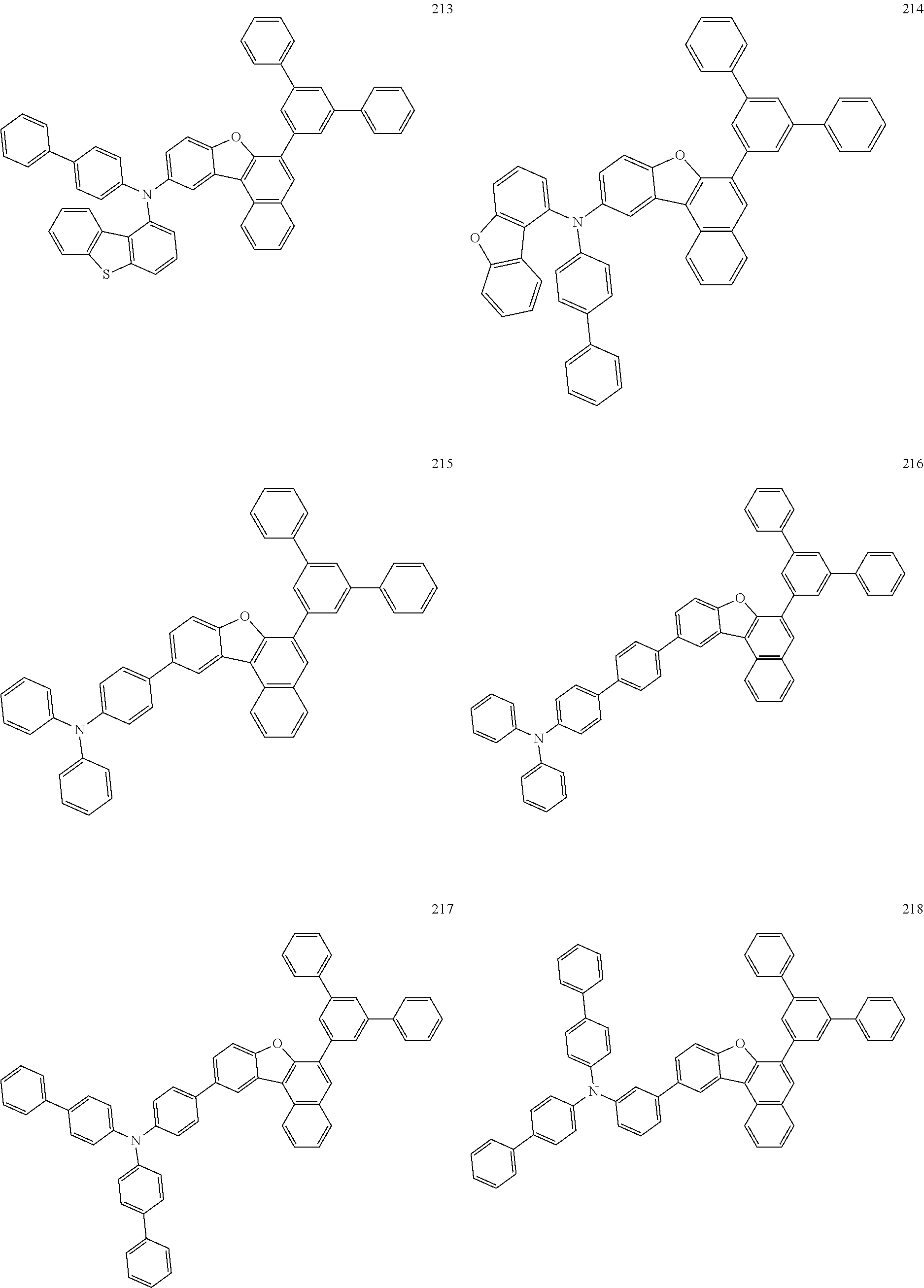

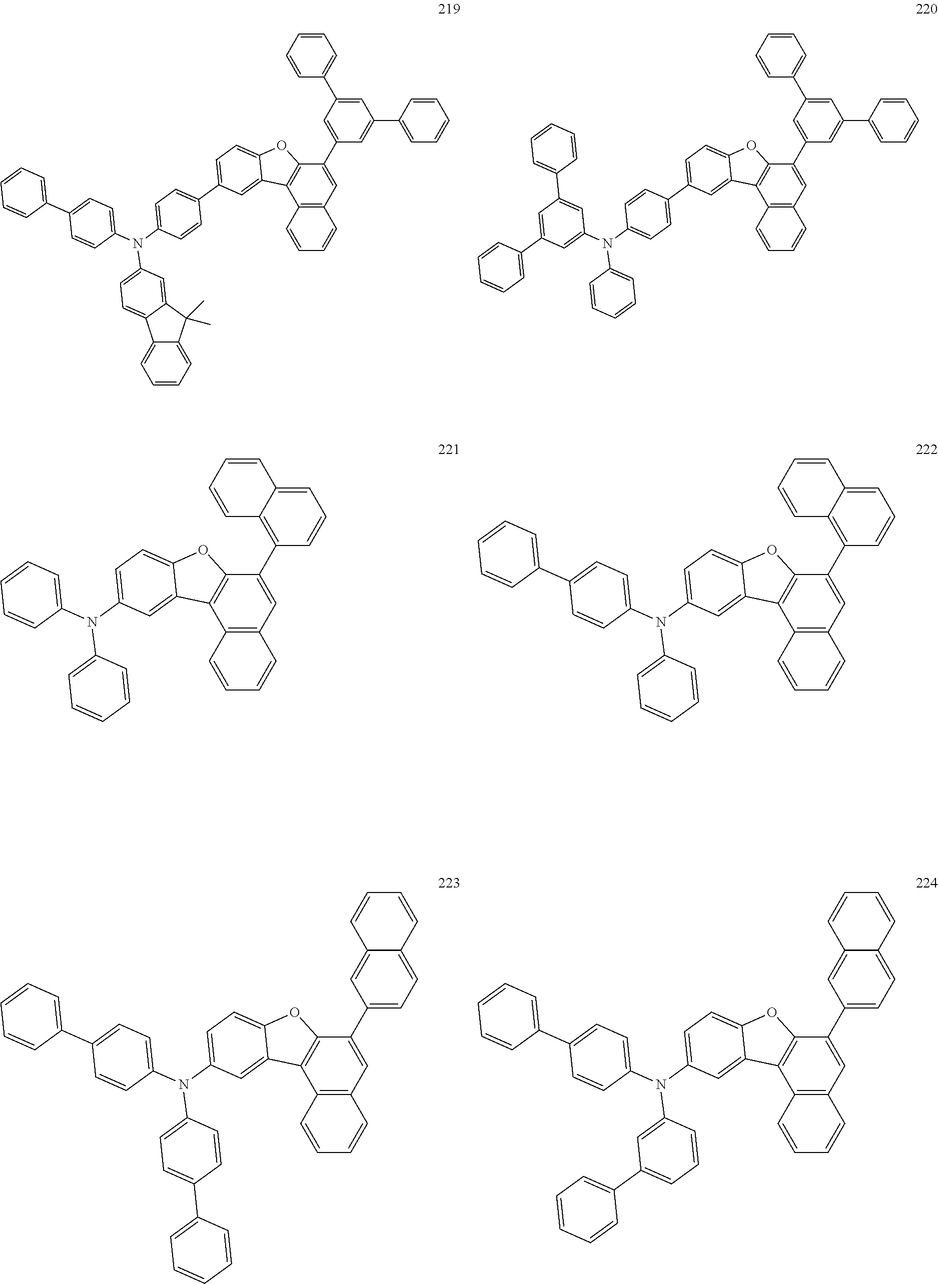

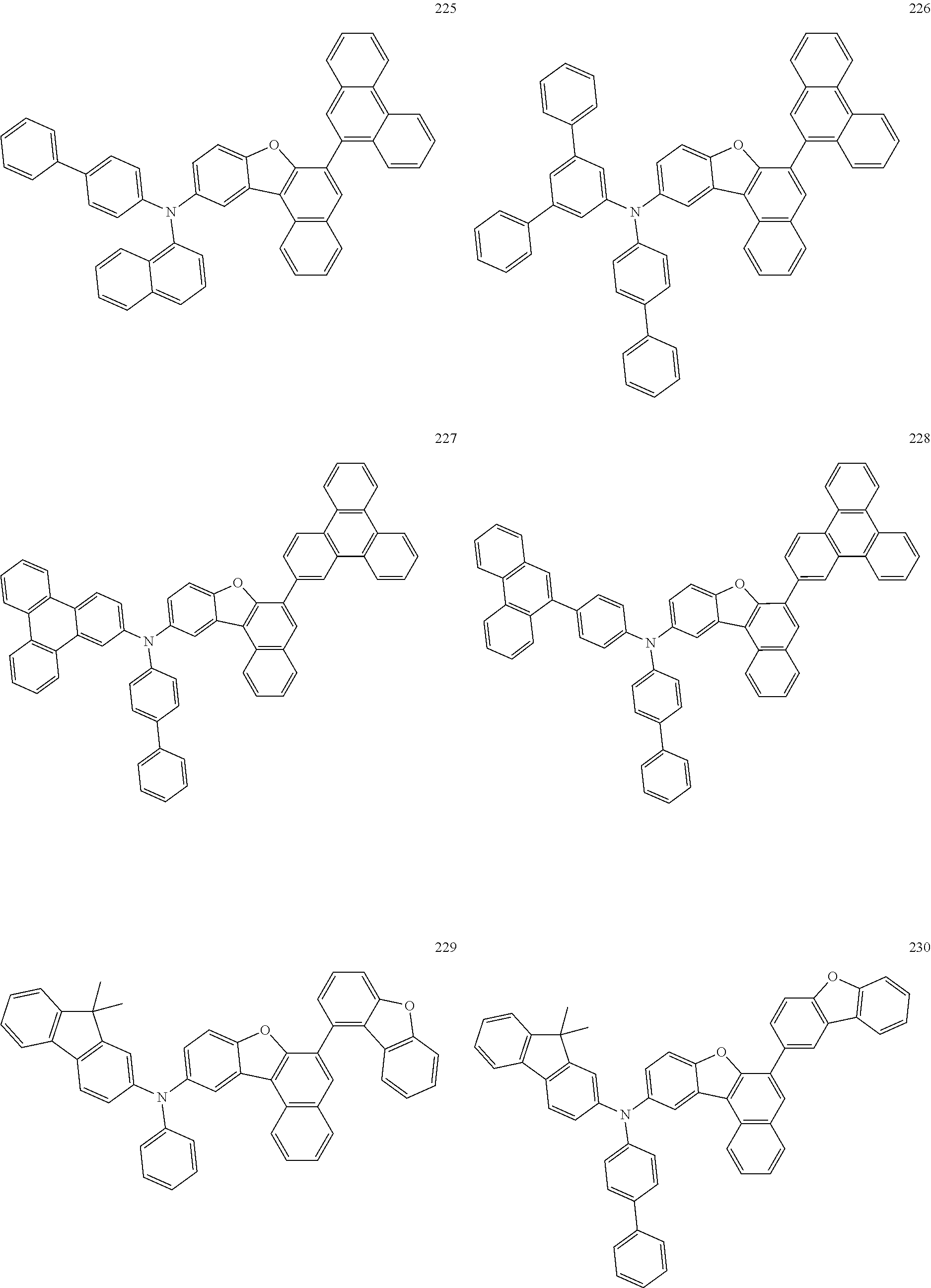

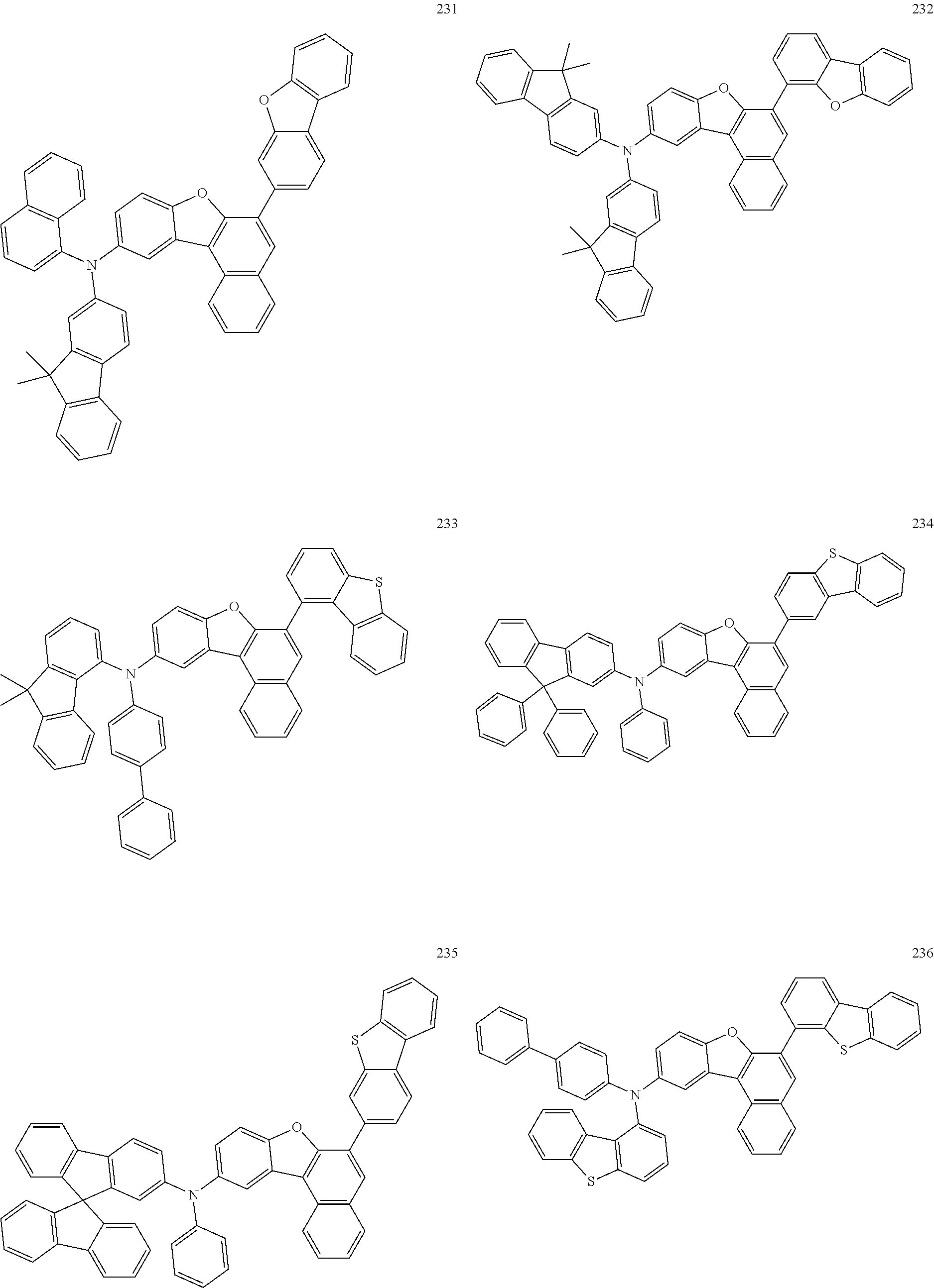

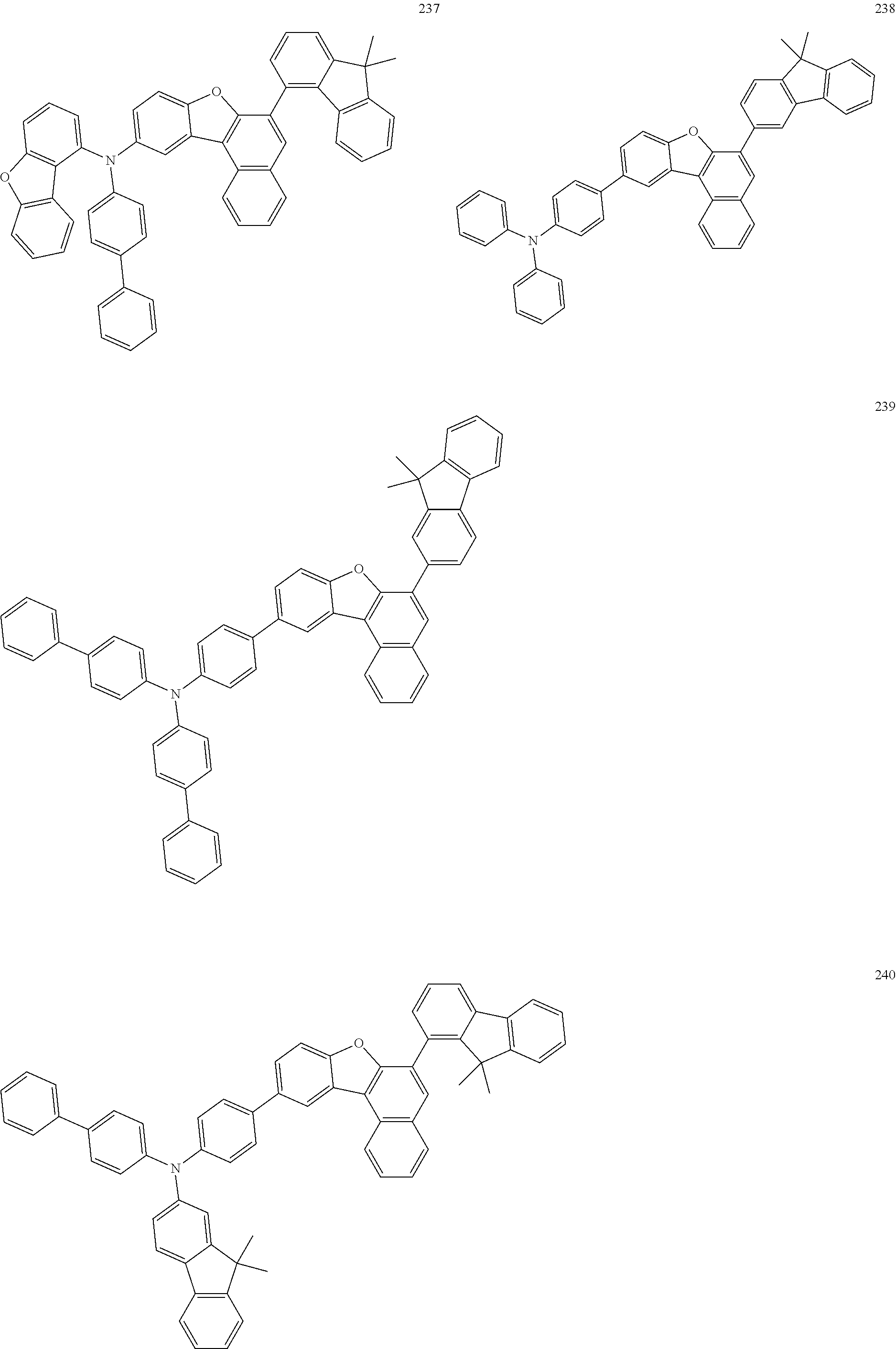 [SMILES: CC1(C)c2ccccc2-c2c(-c3cc4ccccc4c4c3oc3ccc(N(c5ccc(-c6ccccc6)cc5)c5cccc6oc7ccccc7c56)cc34)cccc21 ; CC1(C)c2ccccc2-c2cc(-c3cc4ccccc4c4c3oc3ccc(-c5ccc(N(c6ccccc6)c6ccccc6)cc5)cc34)ccc21 ; CC1(C)c2ccccc2-c2ccc(-c3cc4ccccc4c4c3oc3ccc(-c5ccc(N(c6ccc(-c7ccccc7)cc6)c6ccc(-c7ccccc7)cc6)cc5)cc34)cc21 ; CC1(C)c2ccccc2-c2ccc(N(c3ccc(-c4ccccc4)cc3)c3ccc(-c4ccc5oc6c(-c7cccc8c7C(C)(C)c7ccccc7-8)cc7ccccc7c6c5c4)cc3)cc21]

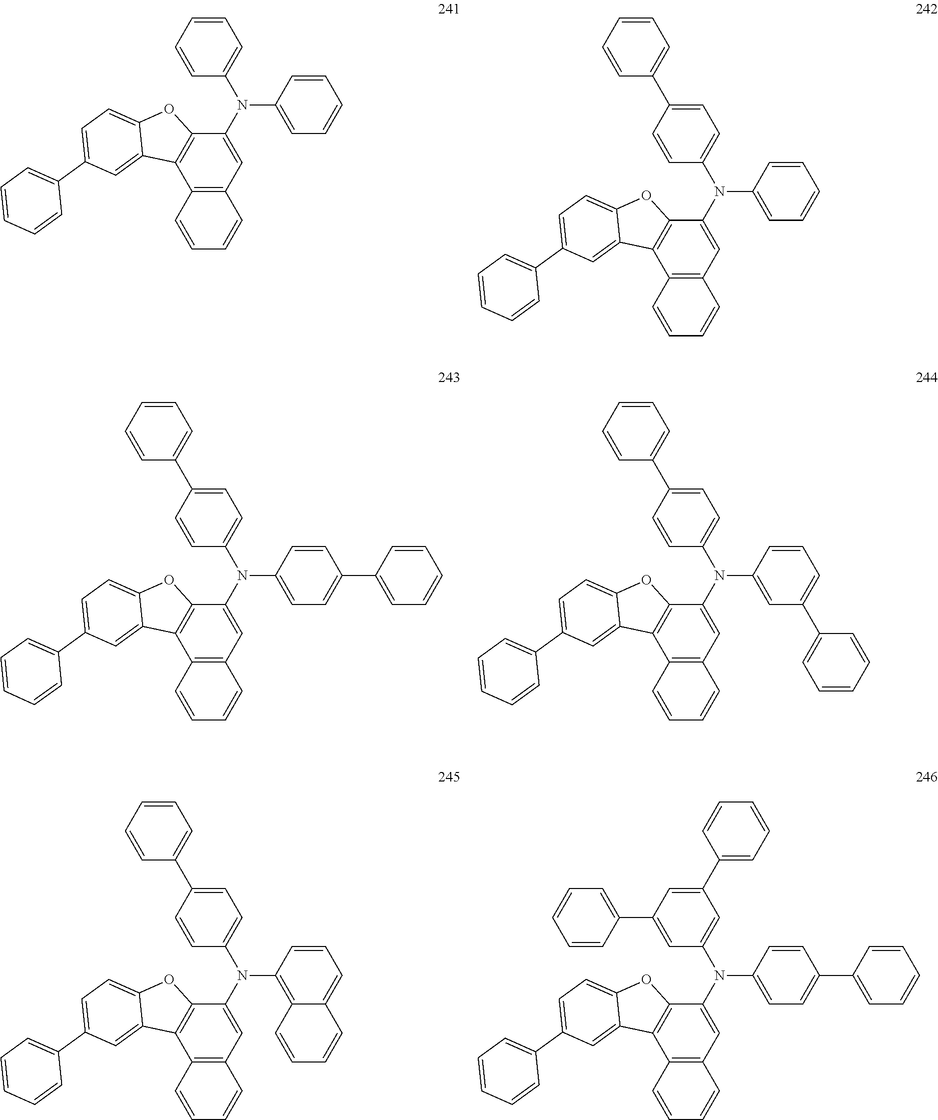 [SMILES: c1ccc(-c2ccc3oc4c(N(c5ccccc5)c5ccccc5)cc5ccccc5c4c3c2)cc1 ; c1ccc(-c2ccc(N(c3ccccc3)c3cc4ccccc4c4c3oc3ccc(-c5ccccc5)cc34)cc2)cc1 ; c1ccc(-c2ccc(N(c3ccc(-c4ccccc4)cc3)c3cc4ccccc4c4c3oc3ccc(-c5ccccc5)cc34)cc2)cc1 ; c1ccc(-c2ccc(N(c3cccc(-c4ccccc4)c3)c3cc4ccccc4c4c3oc3ccc(-c5ccccc5)cc34)cc2)cc1 ; c1ccc(-c2ccc(N(c3cccc4ccccc34)c3cc4ccccc4c4c3oc3ccc(-c5ccccc5)cc34)cc2)cc1 ; c1ccc(-c2ccc(N(c3cc(-c4ccccc4)cc(-c4ccccc4)c3)c3cc4ccccc4c4c3oc3ccc(-c5ccccc5)cc34)cc2)cc1]

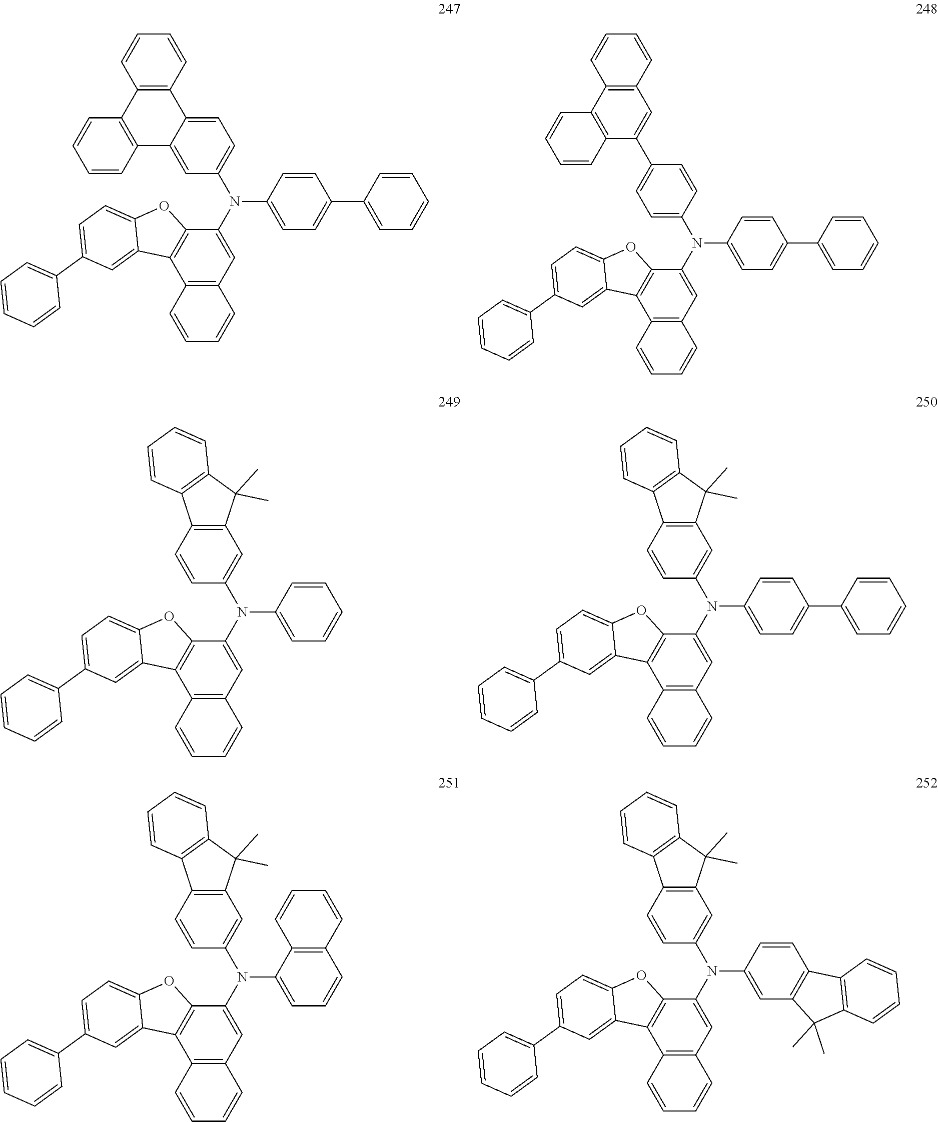

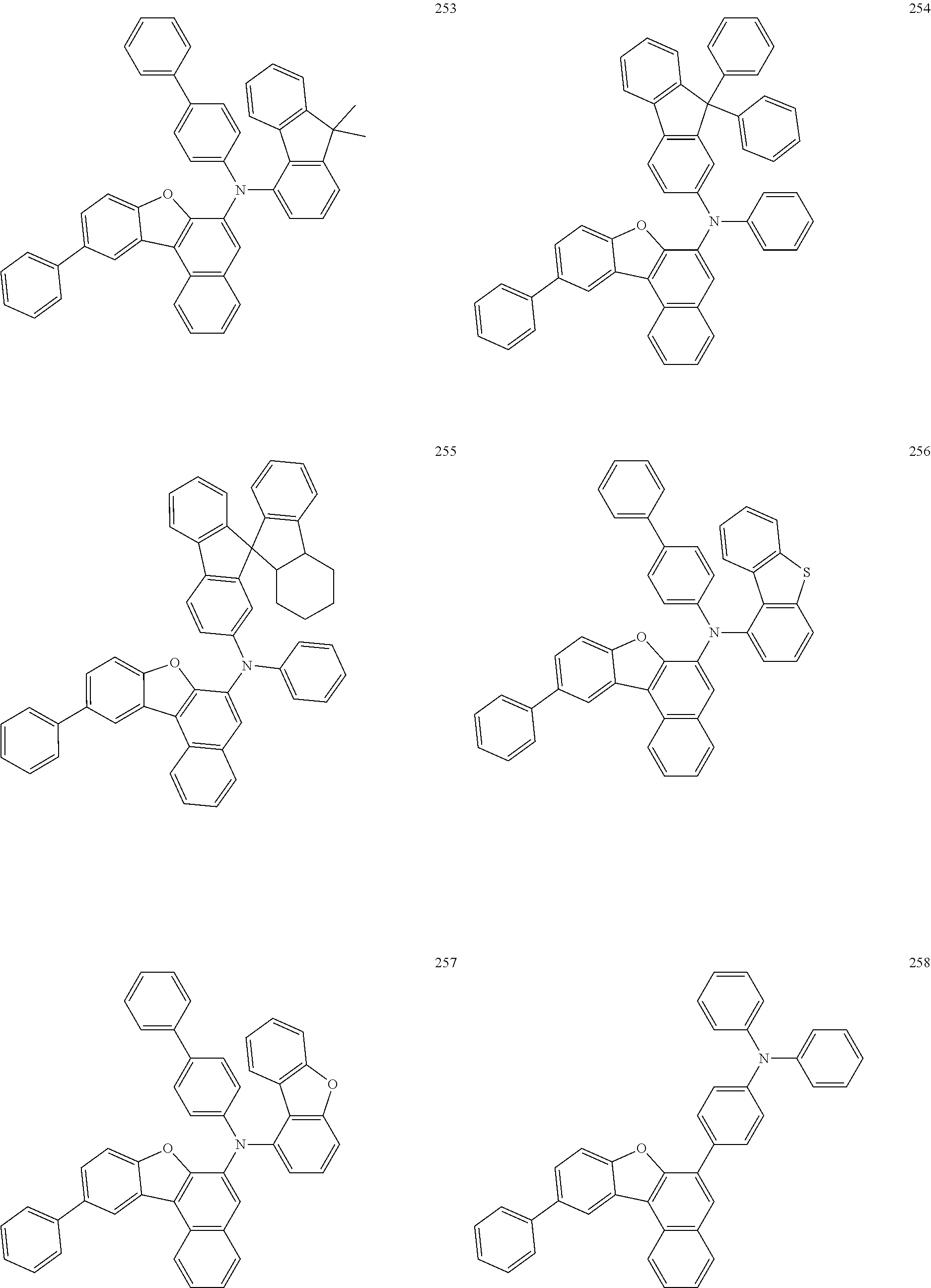

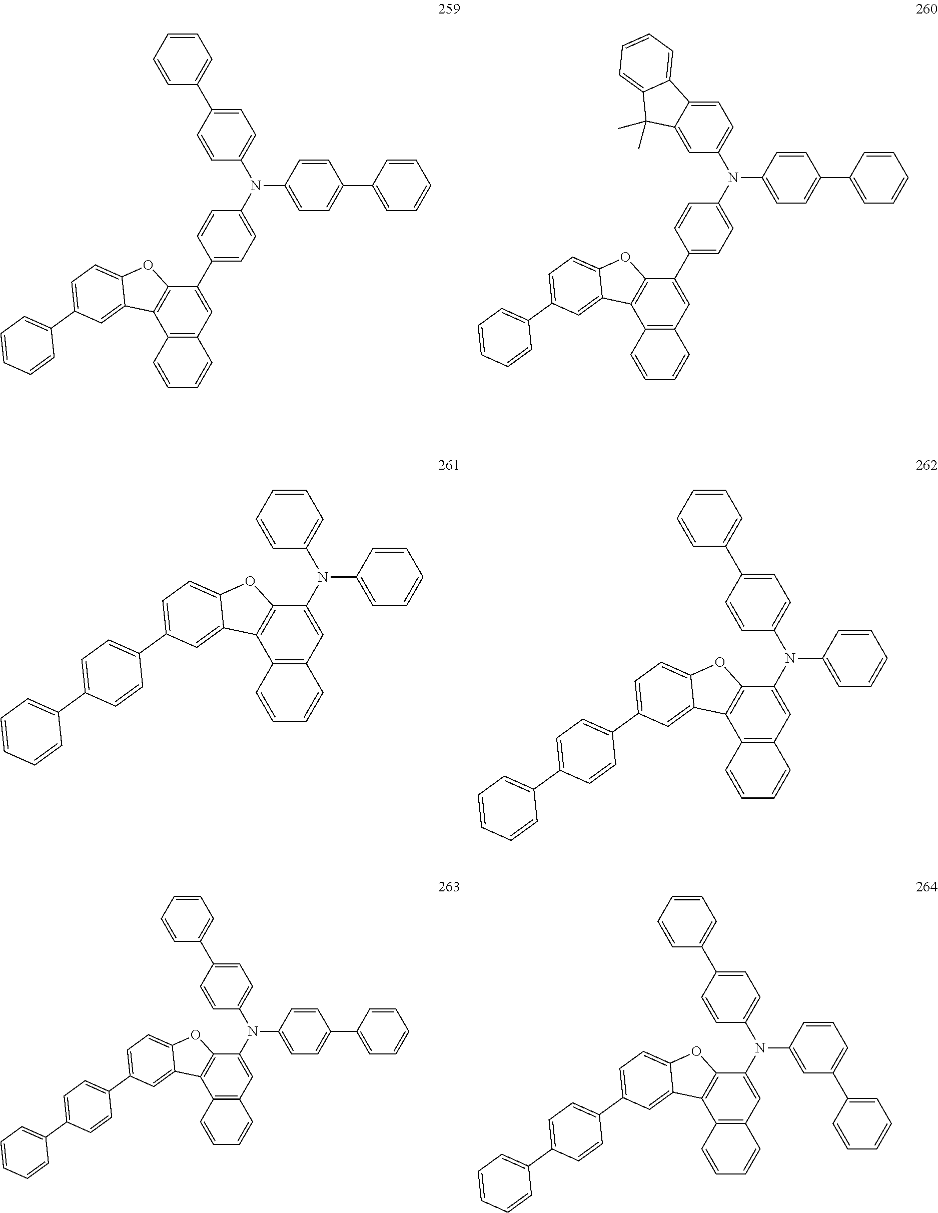

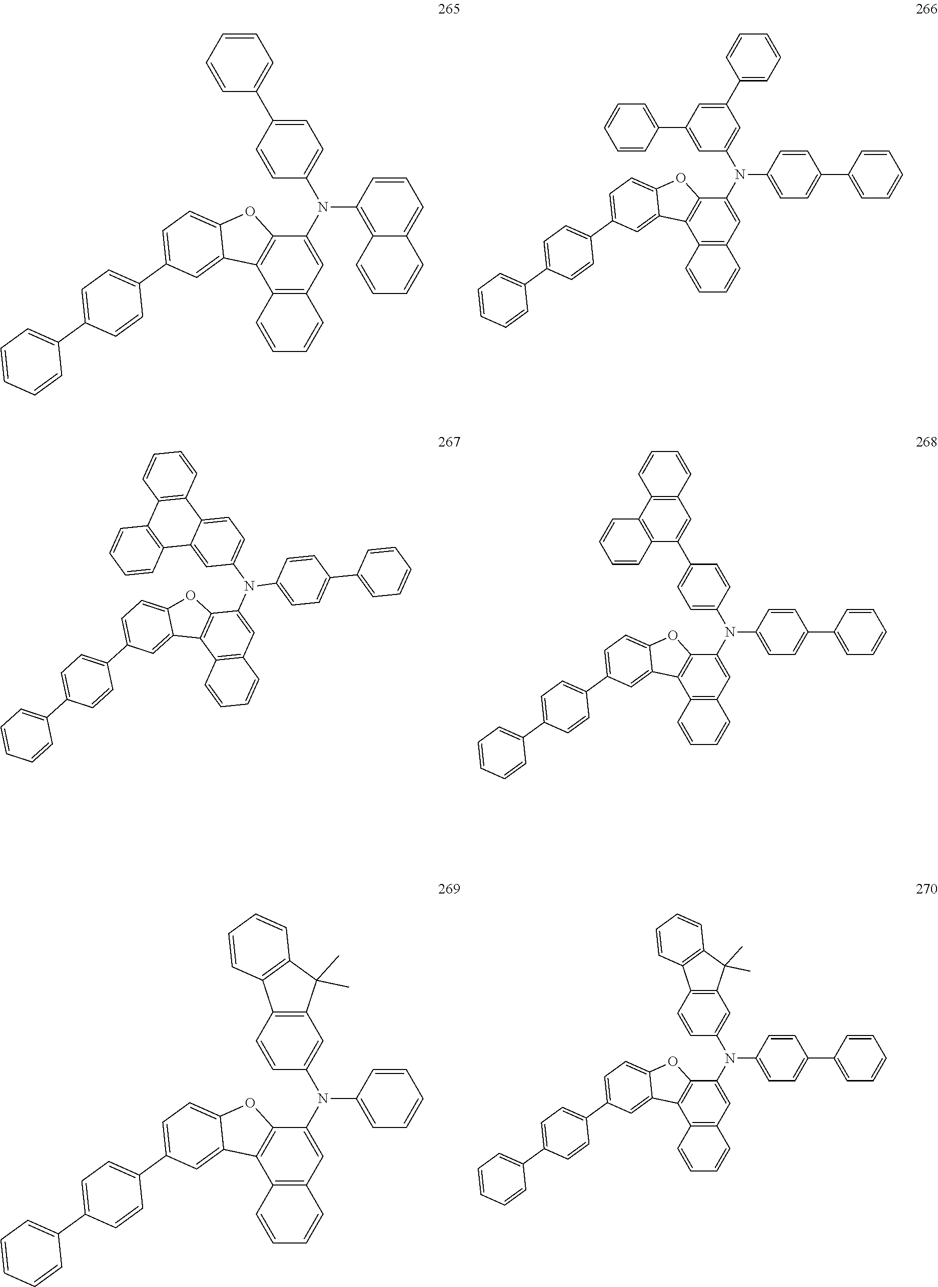

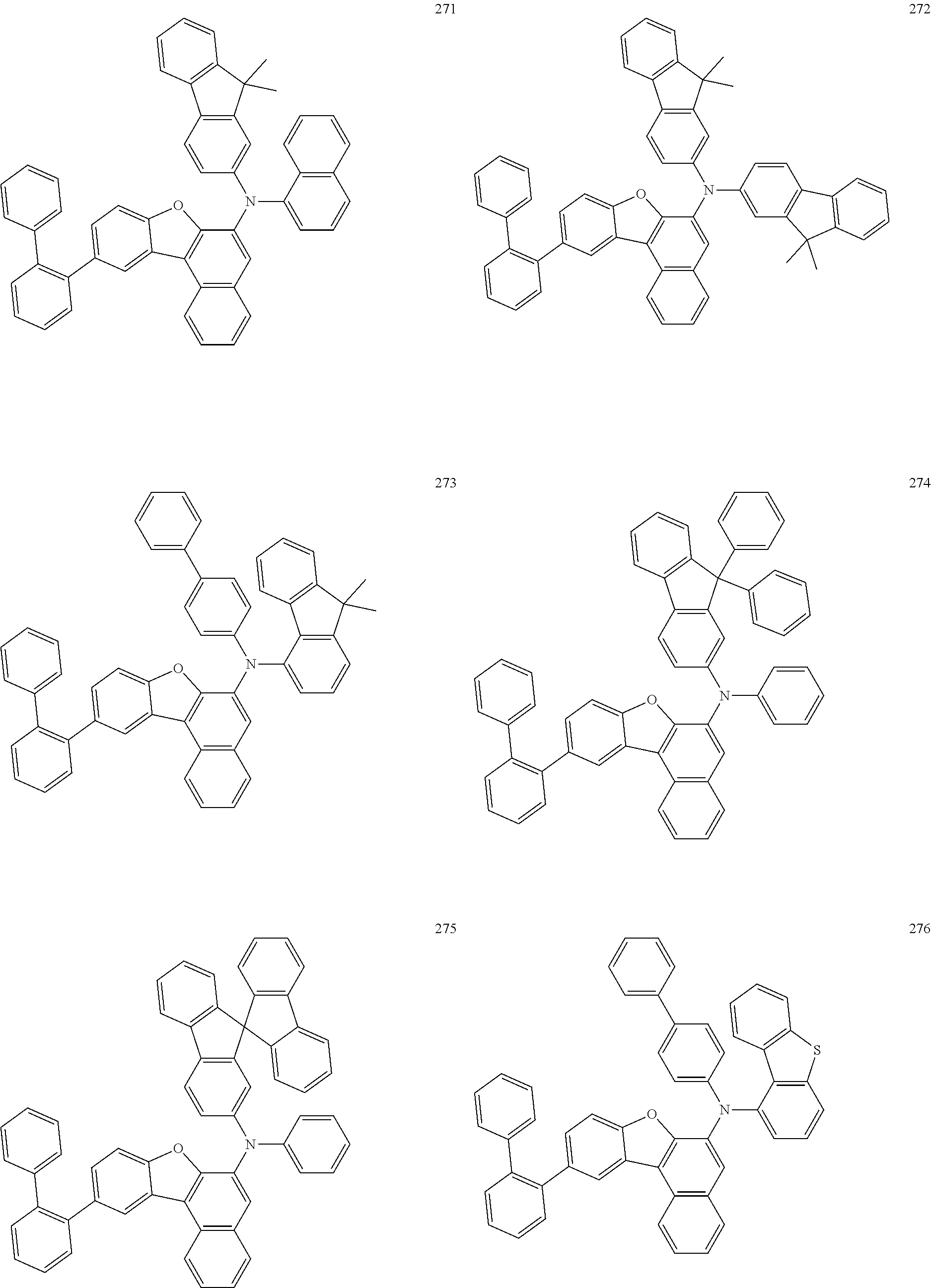

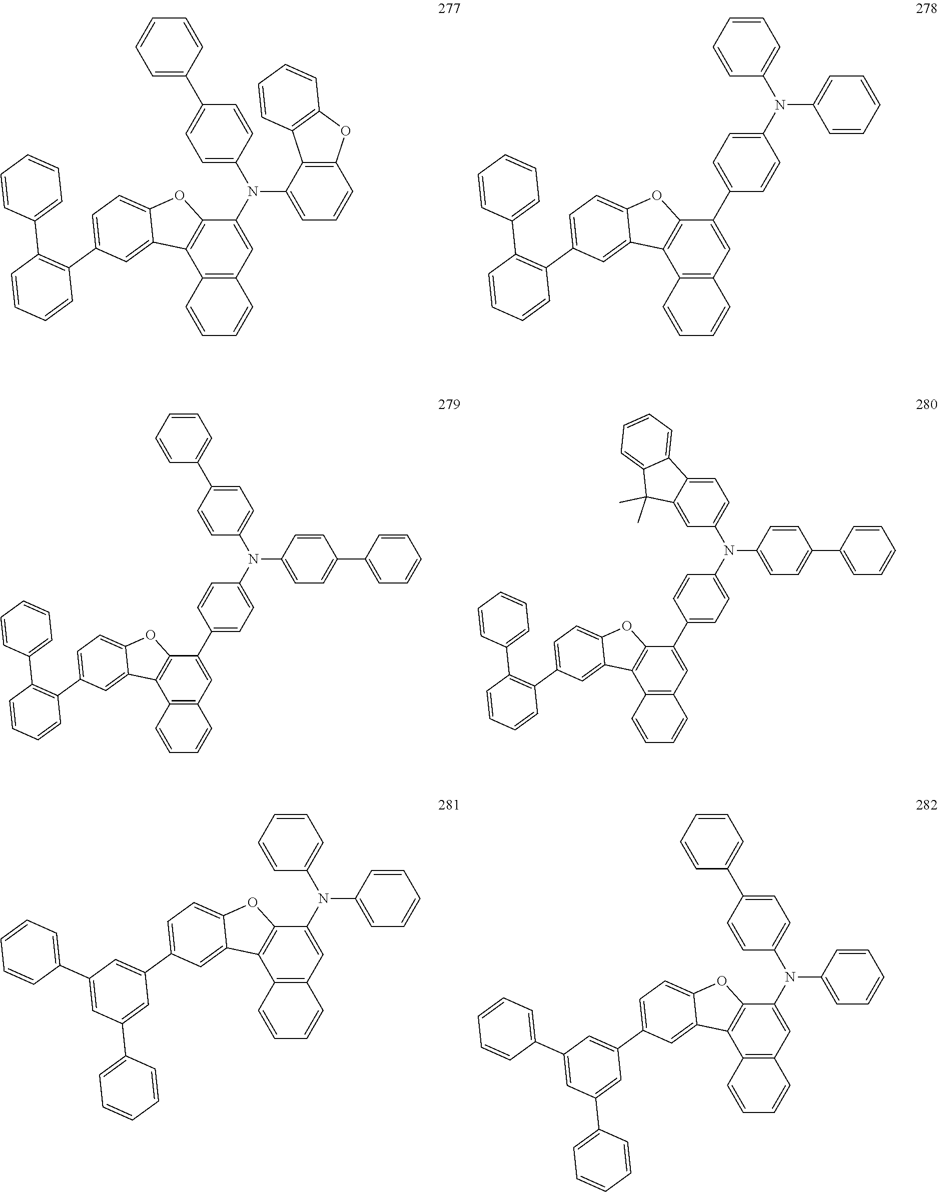

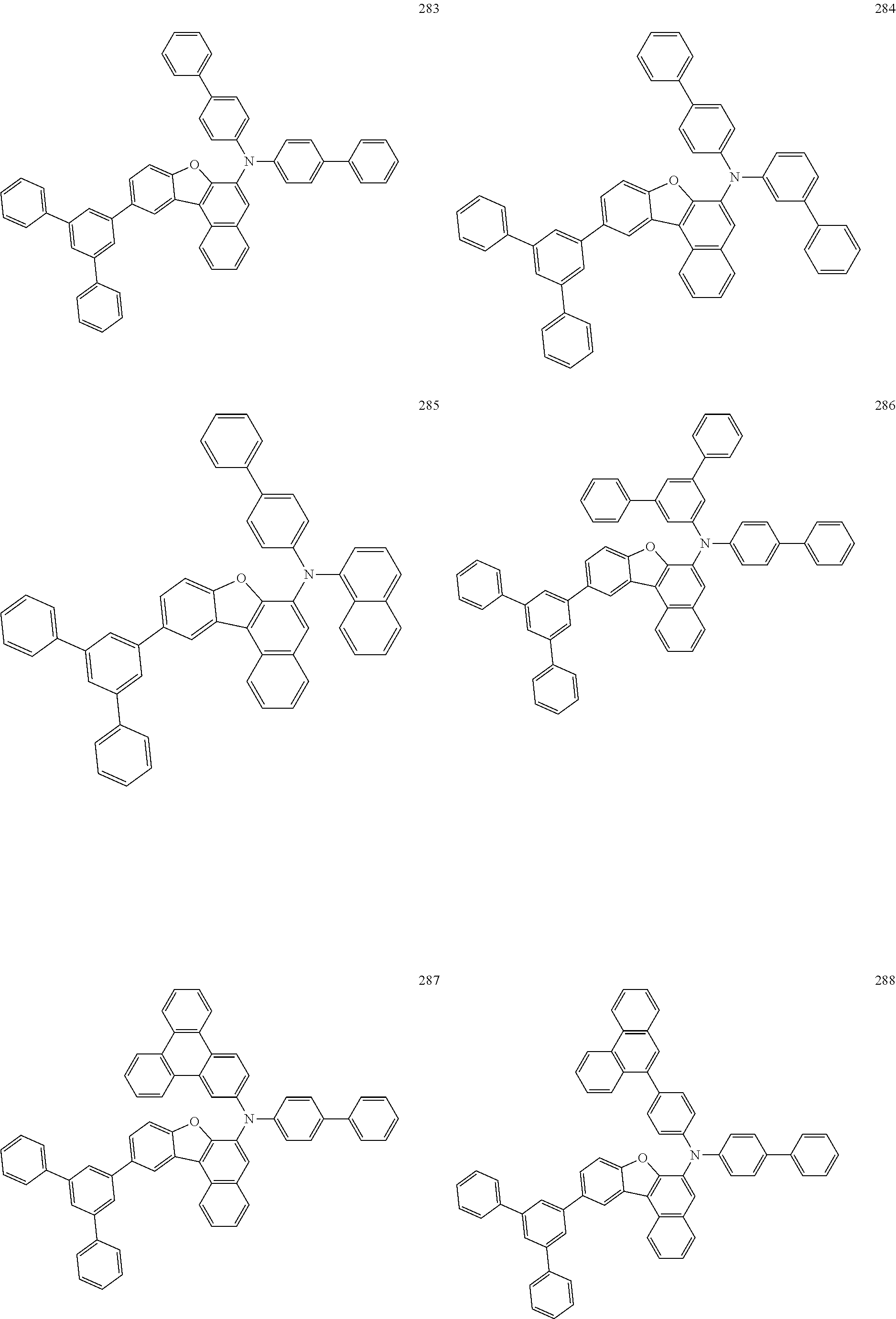

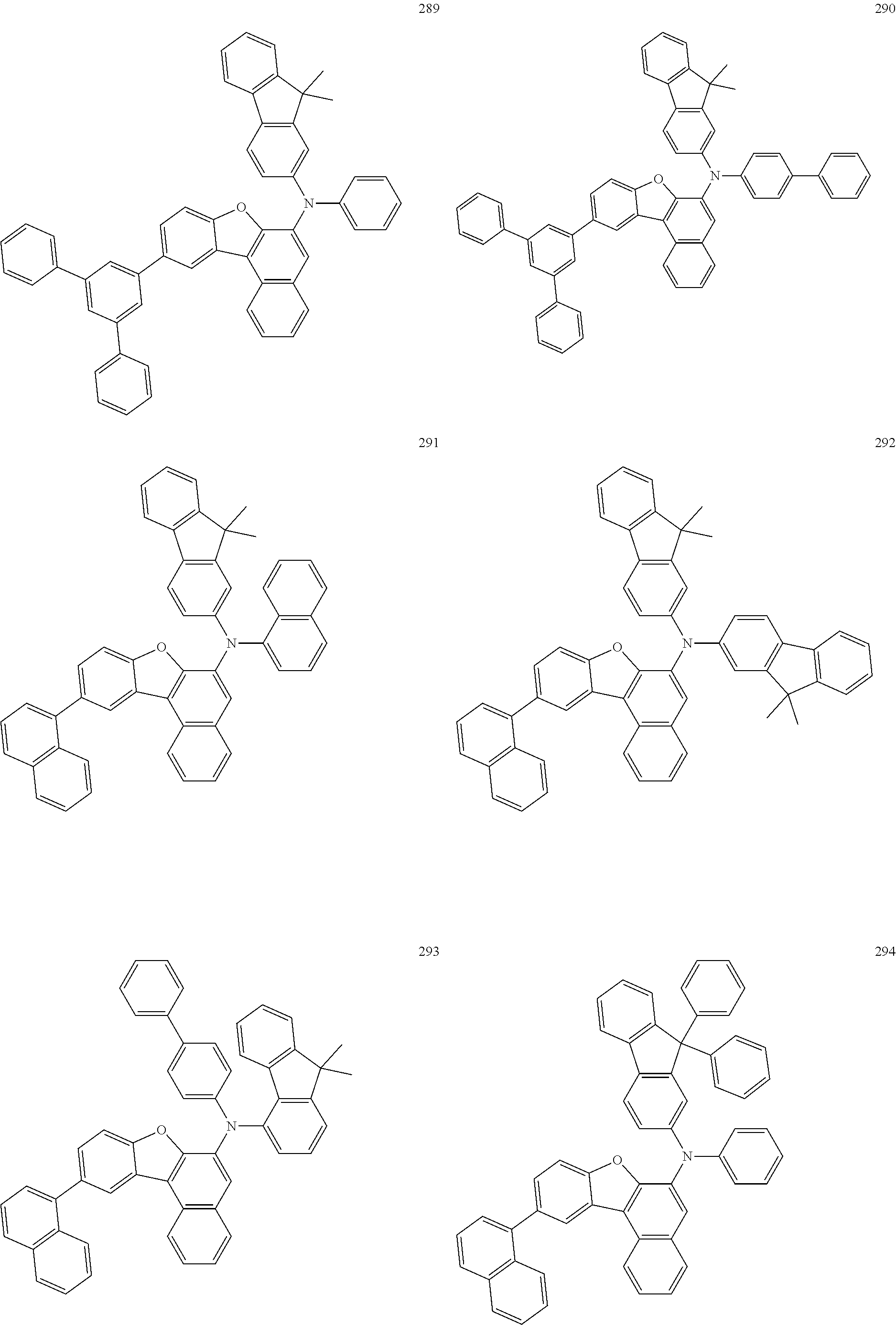

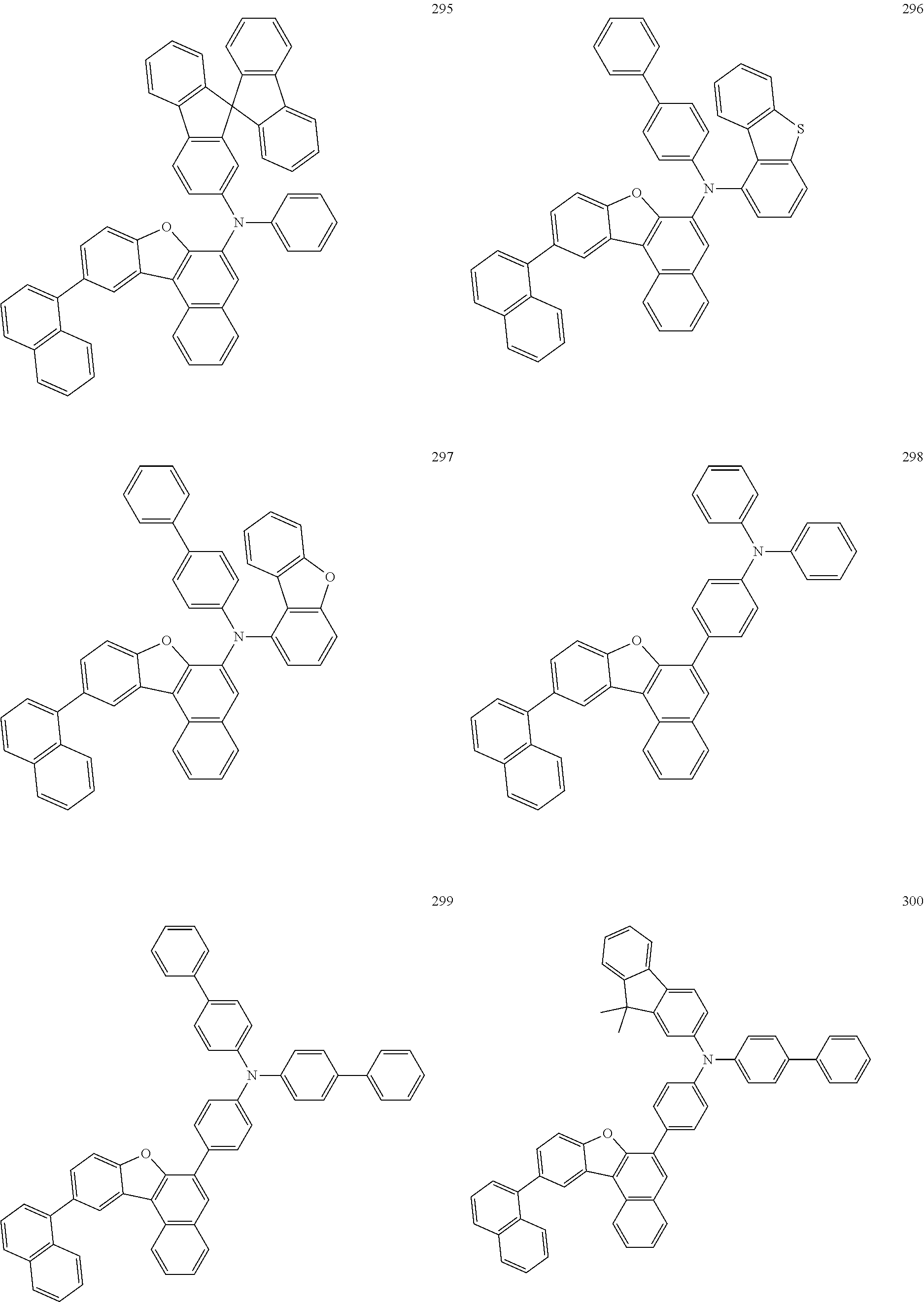

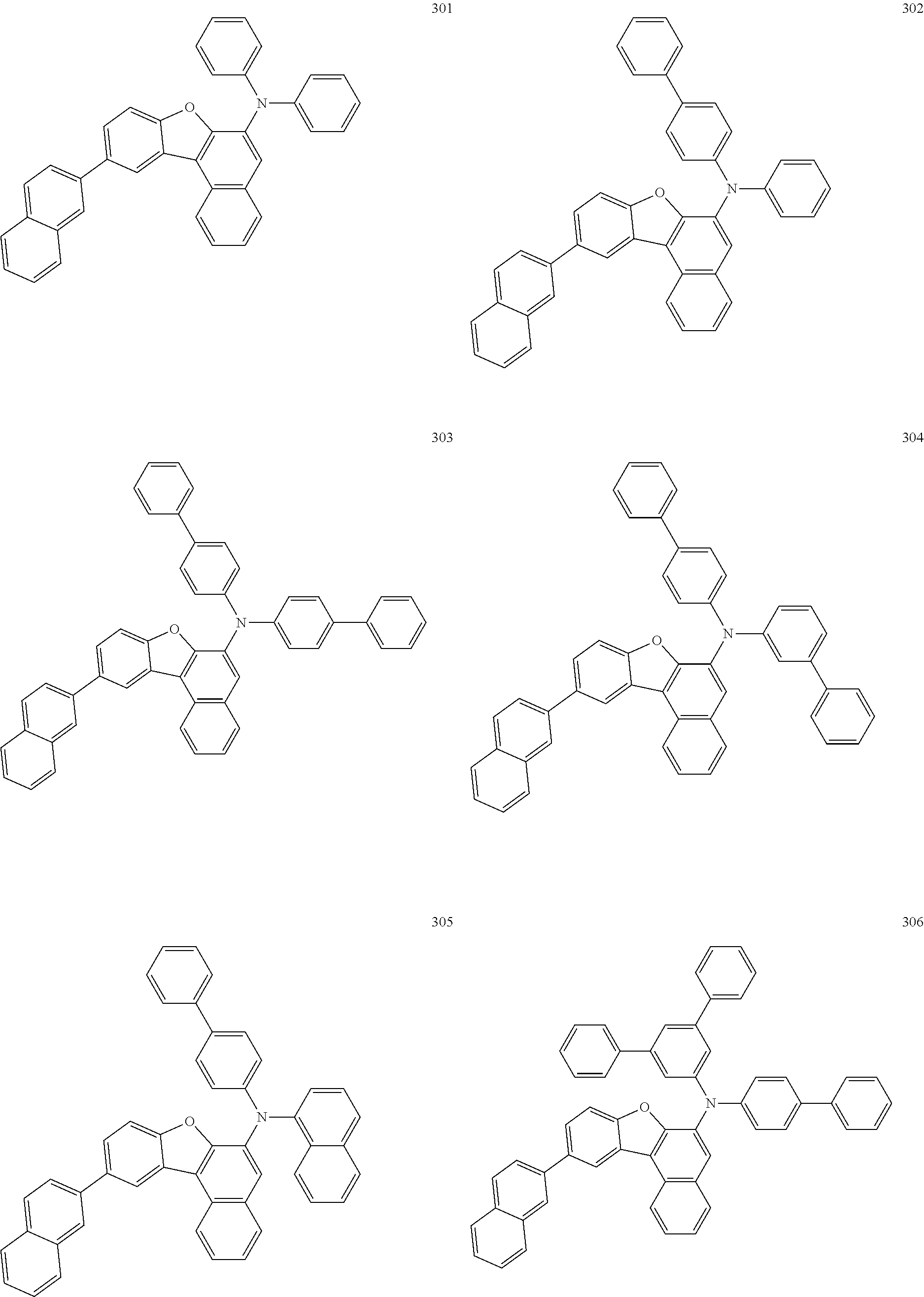 [SMILES: c1ccc(N(c2ccccc2)c2cc3ccccc3c3c2oc2ccc(-c4ccc5ccccc5c4)cc23)cc1 ; c1ccc(-c2ccc(N(c3ccccc3)c3cc4ccccc4c4c3oc3ccc(-c5ccc6ccccc6c5)cc34)cc2)cc1 ; c1ccc(-c2ccc(N(c3ccc(-c4ccccc4)cc3)c3cc4ccccc4c4c3oc3ccc(-c5ccc6ccccc6c5)cc34)cc2)cc1 ; c1ccc(-c2ccc(N(c3cccc(-c4ccccc4)c3)c3cc4ccccc4c4c3oc3ccc(-c5ccc6ccccc6c5)cc34)cc2)cc1 ; c1ccc(-c2ccc(N(c3cccc4ccccc34)c3cc4ccccc4c4c3oc3ccc(-c5ccc6ccccc6c5)cc34)cc2)cc1 ; c1ccc(-c2ccc(N(c3cc(-c4ccccc4)cc(-c4ccccc4)c3)c3cc4ccccc4c4c3oc3ccc(-c5ccc6ccccc6c5)cc34)cc2)cc1]

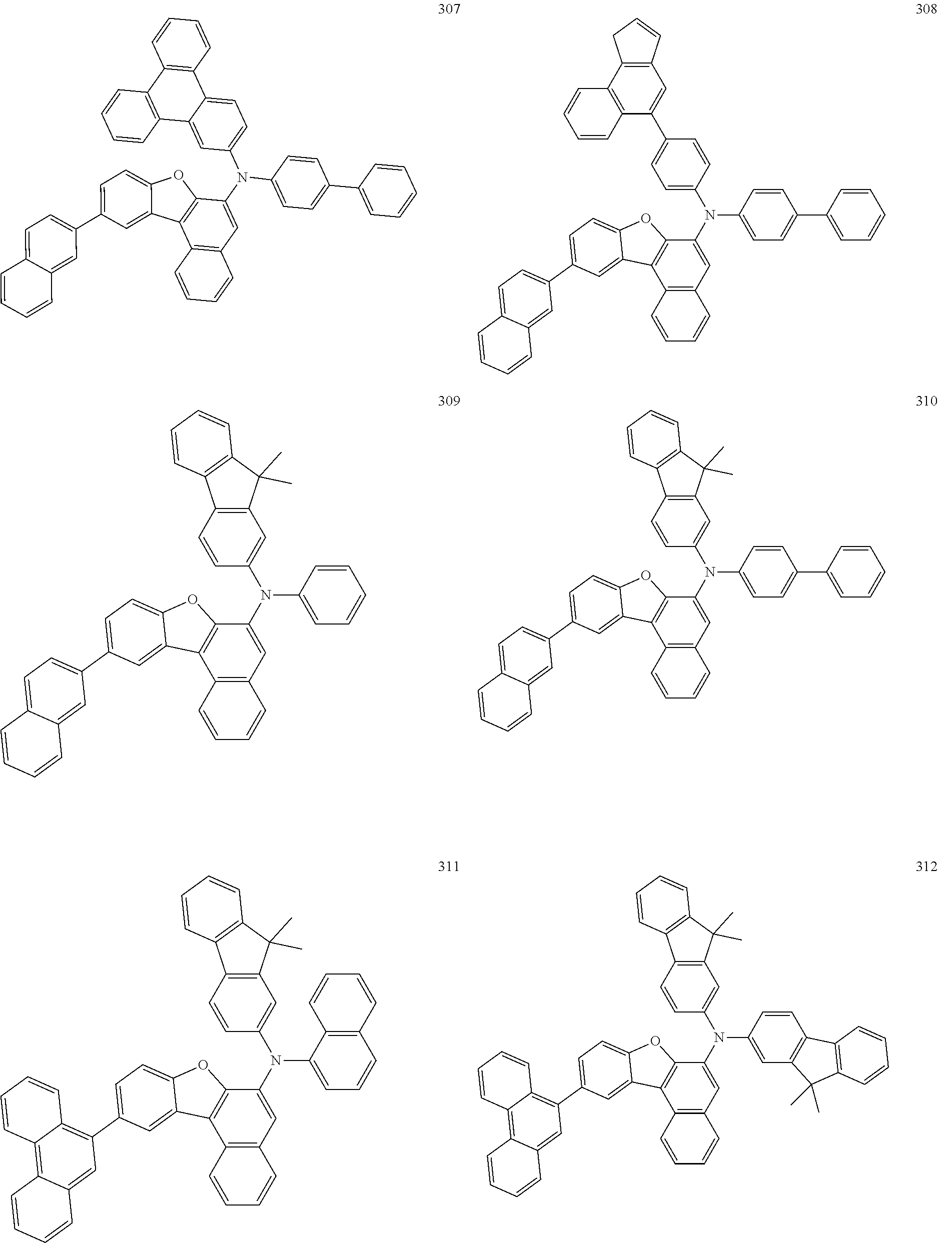

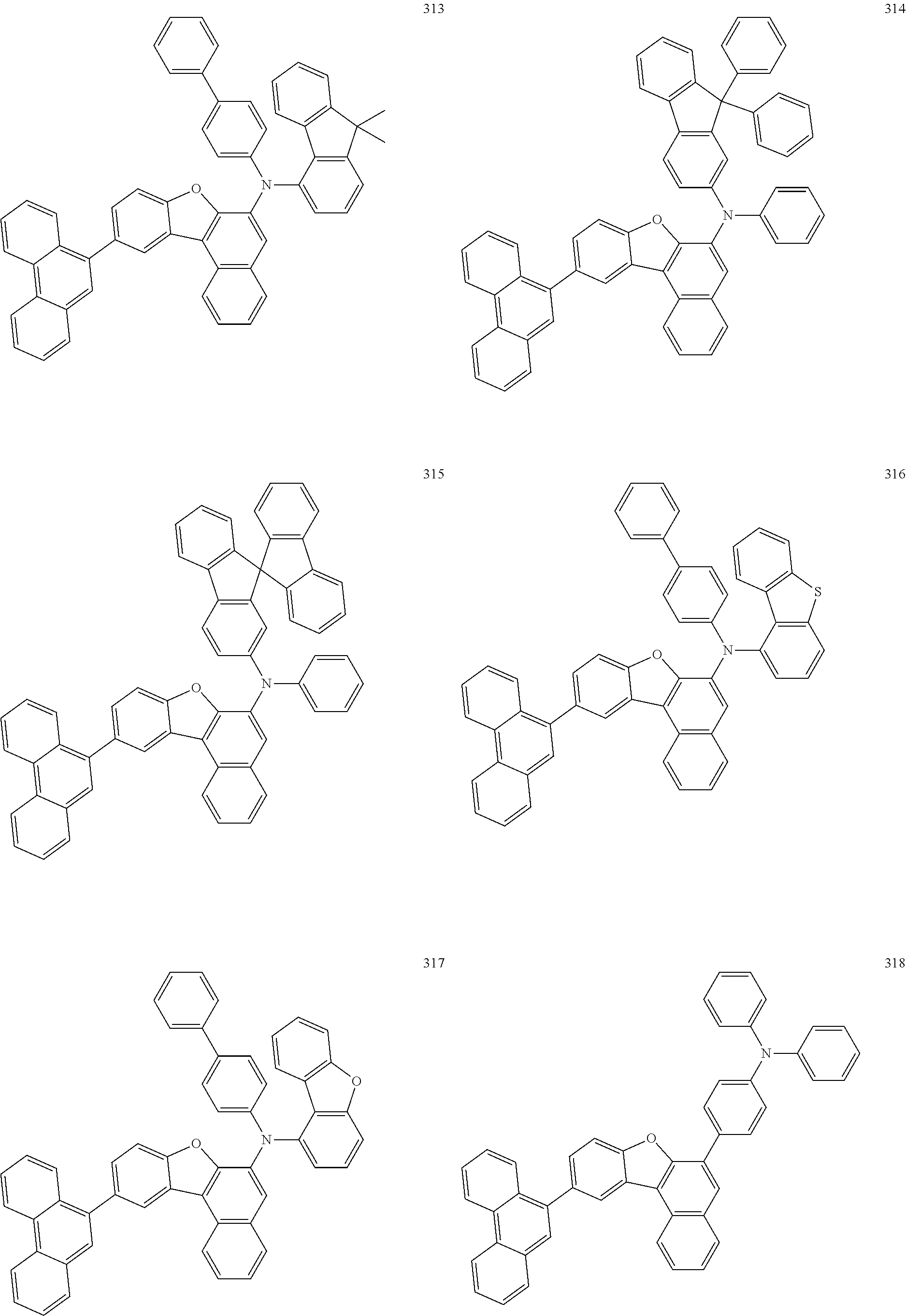

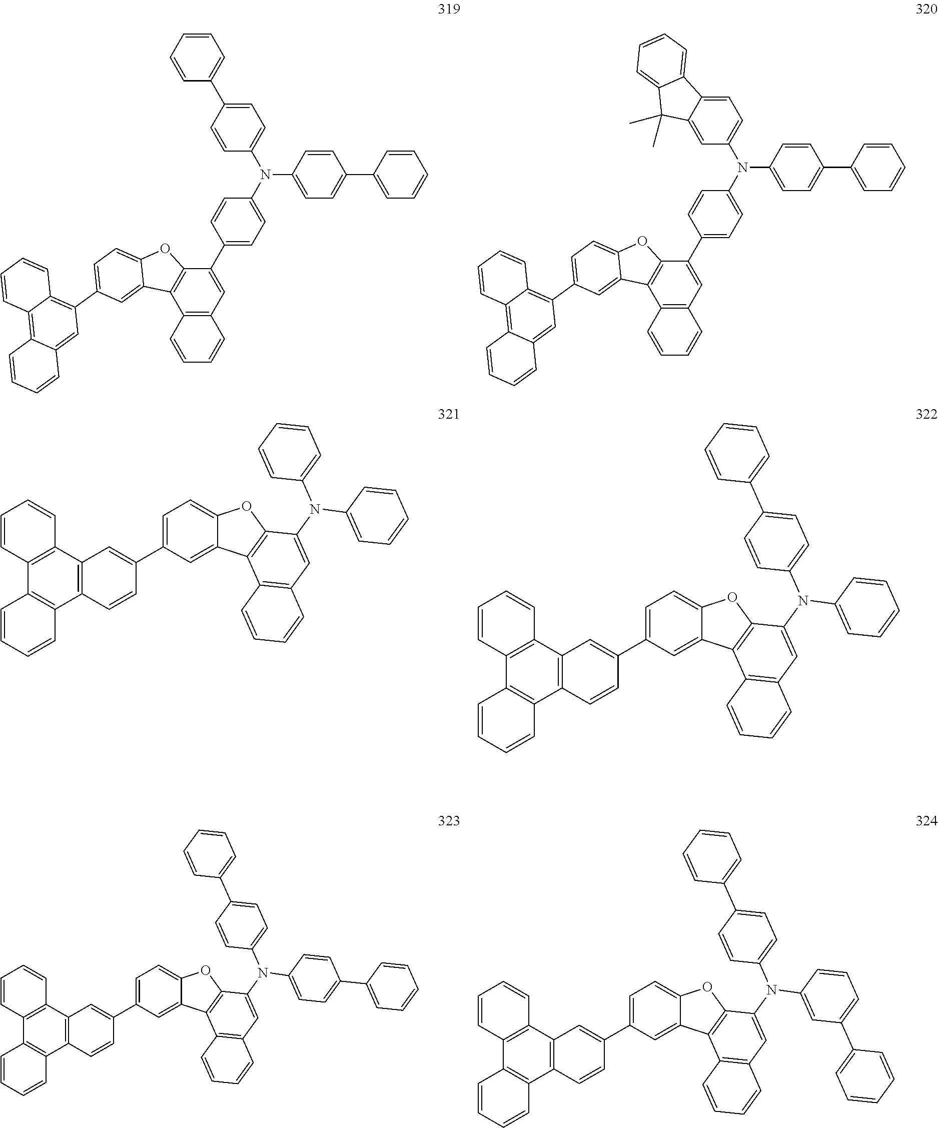

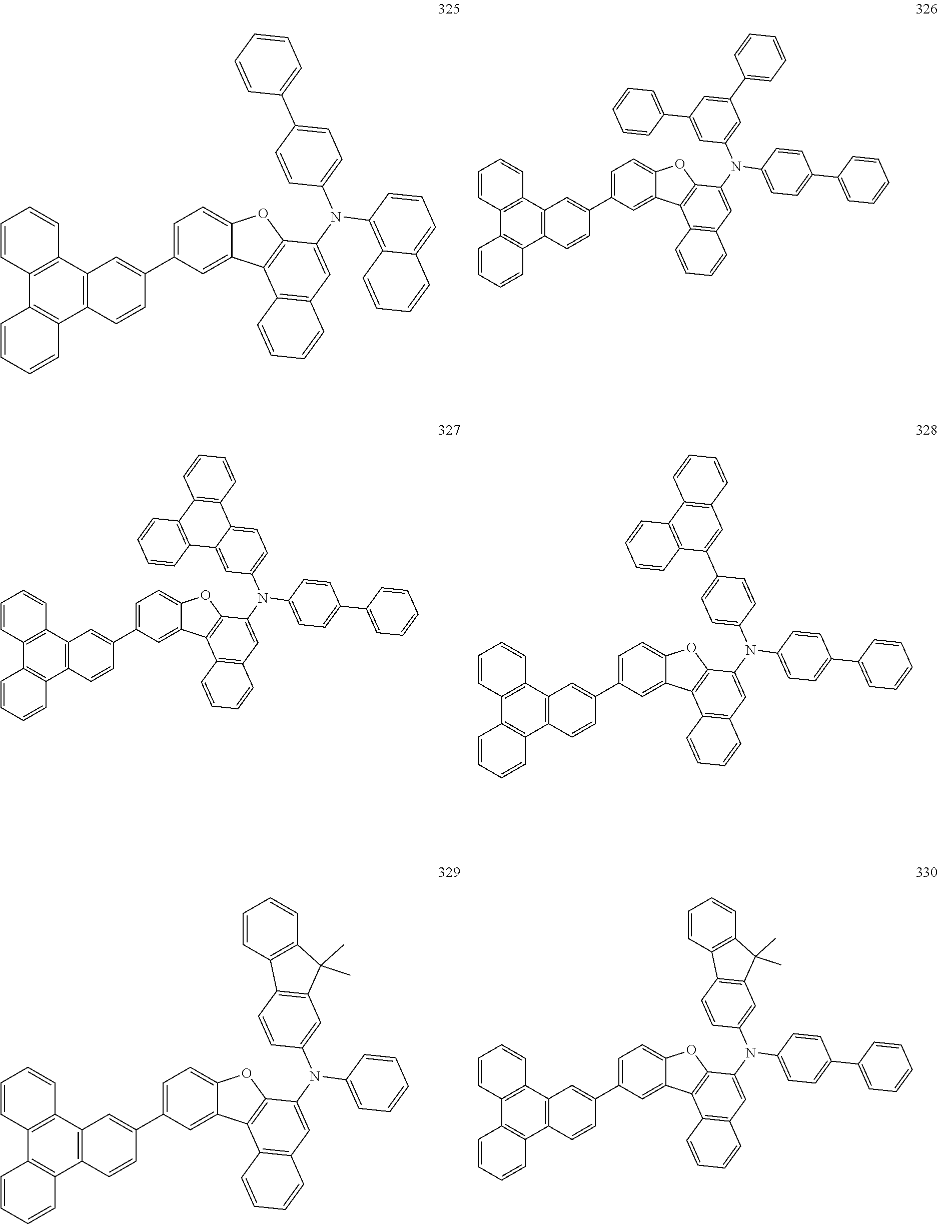

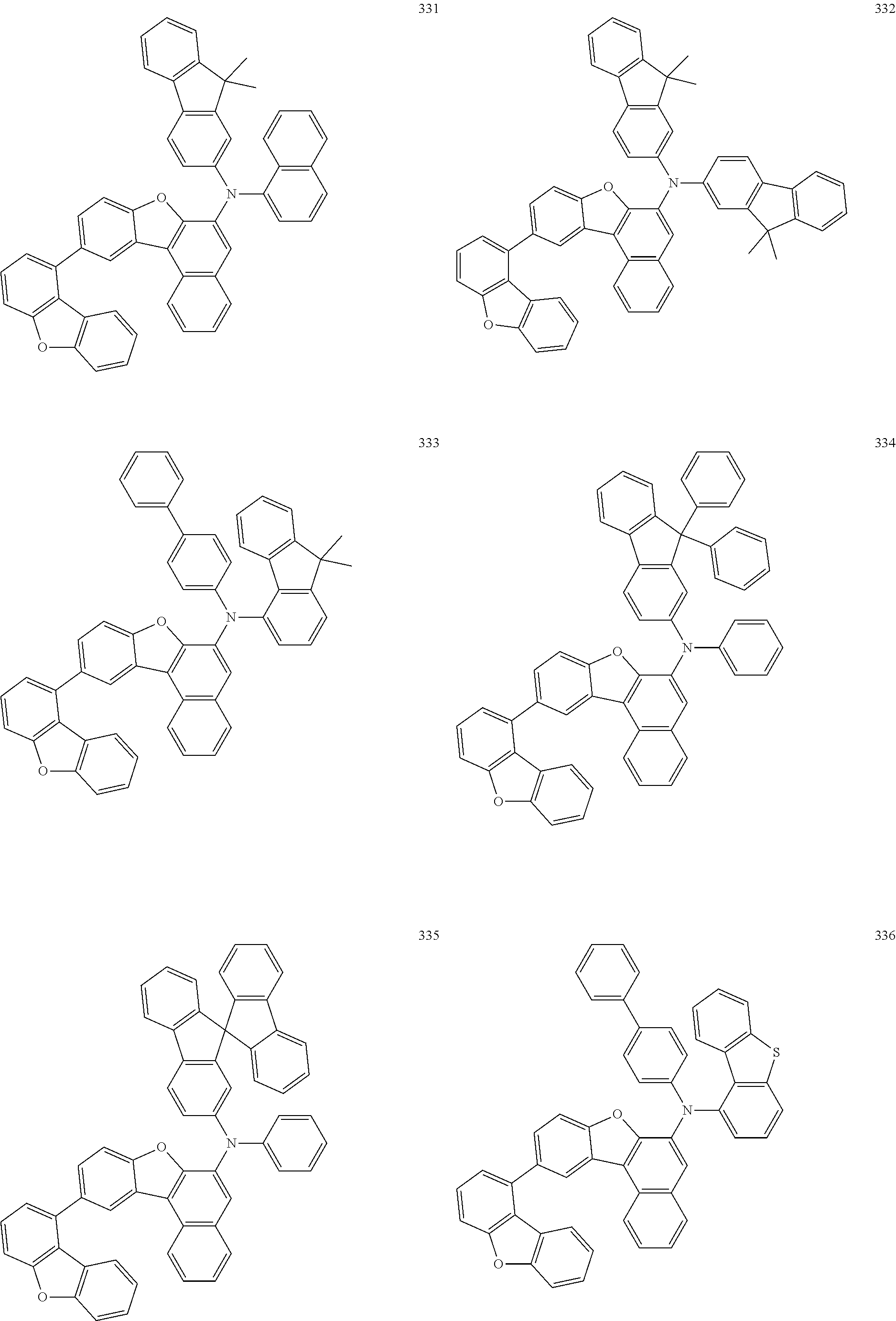

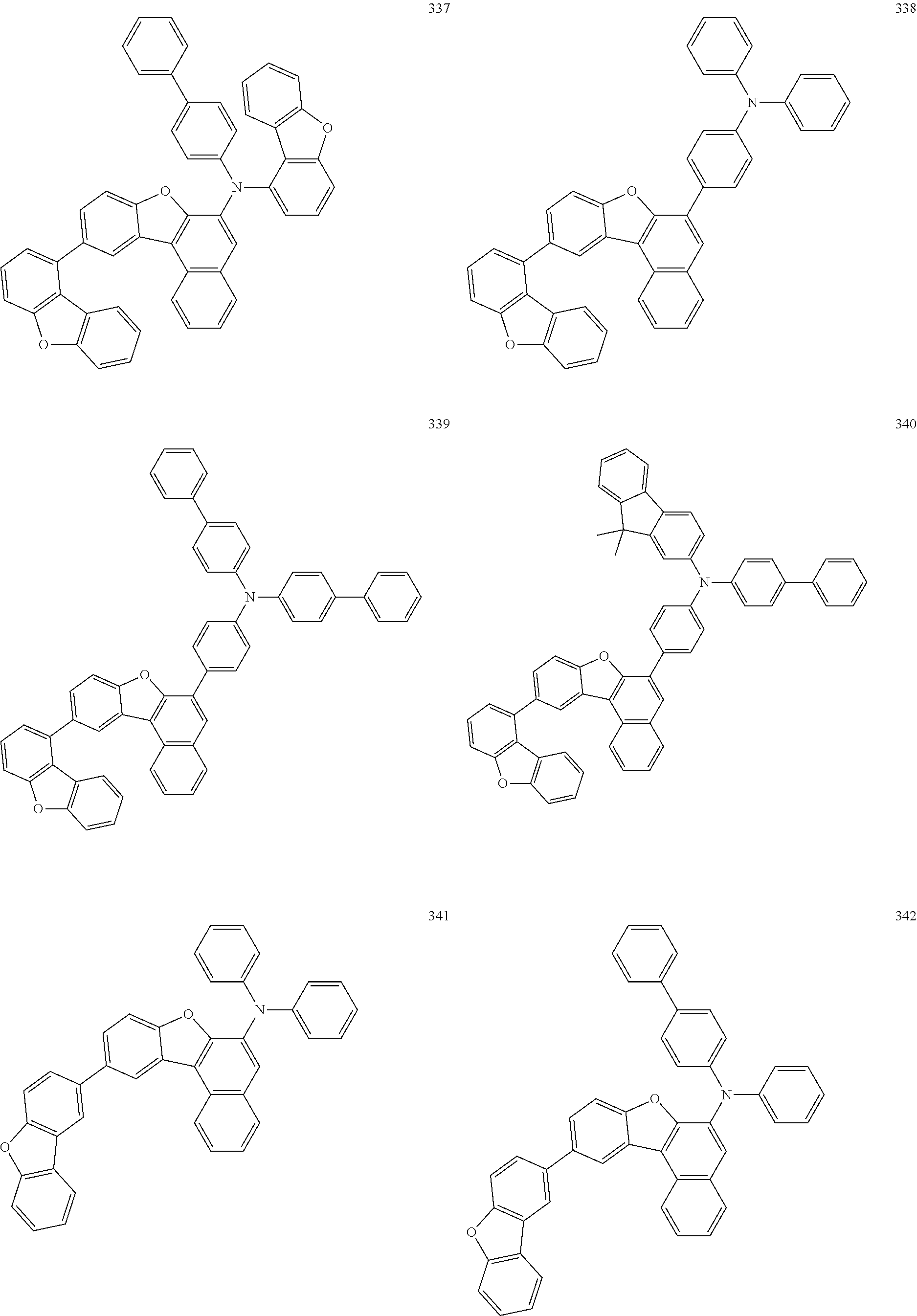

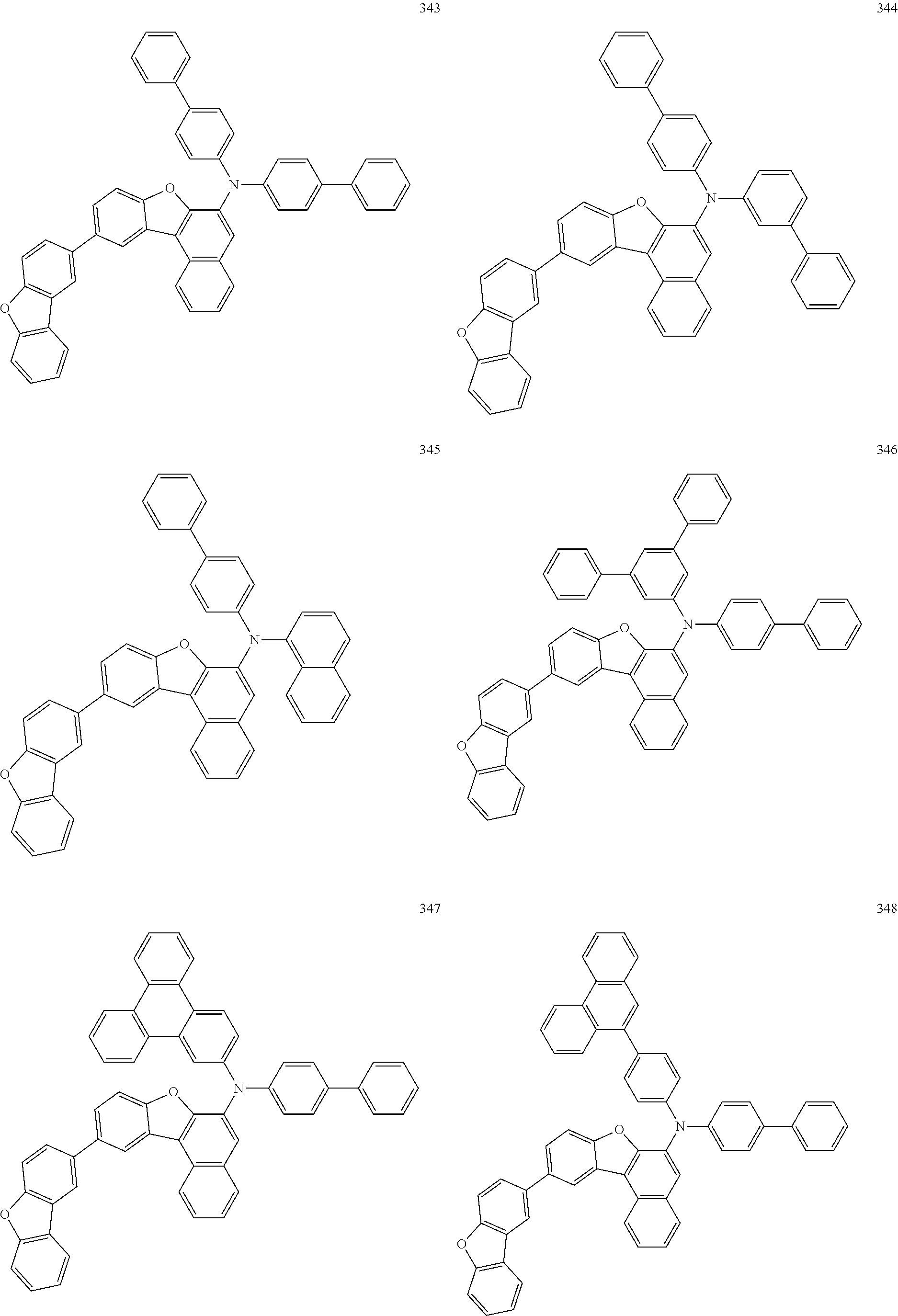

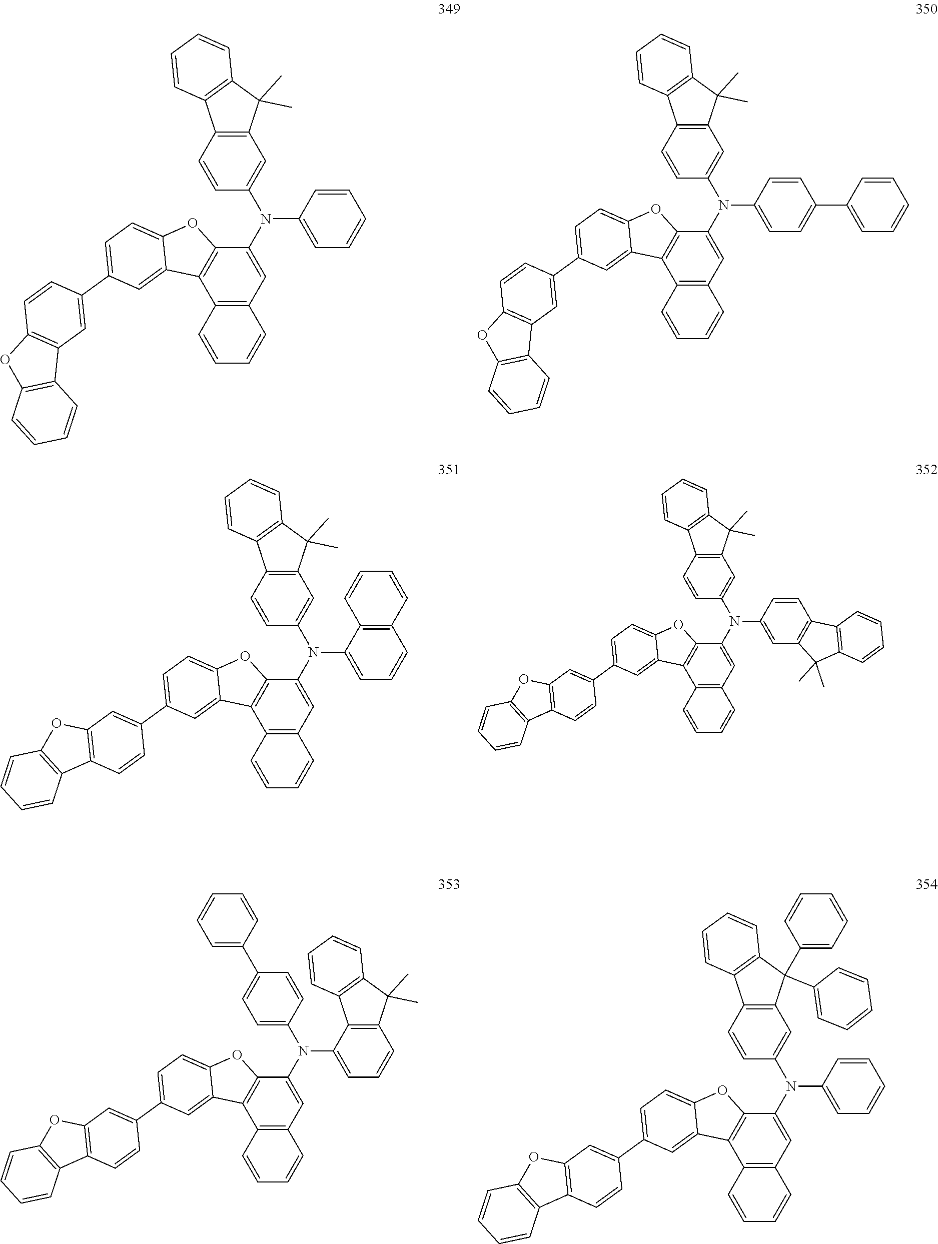

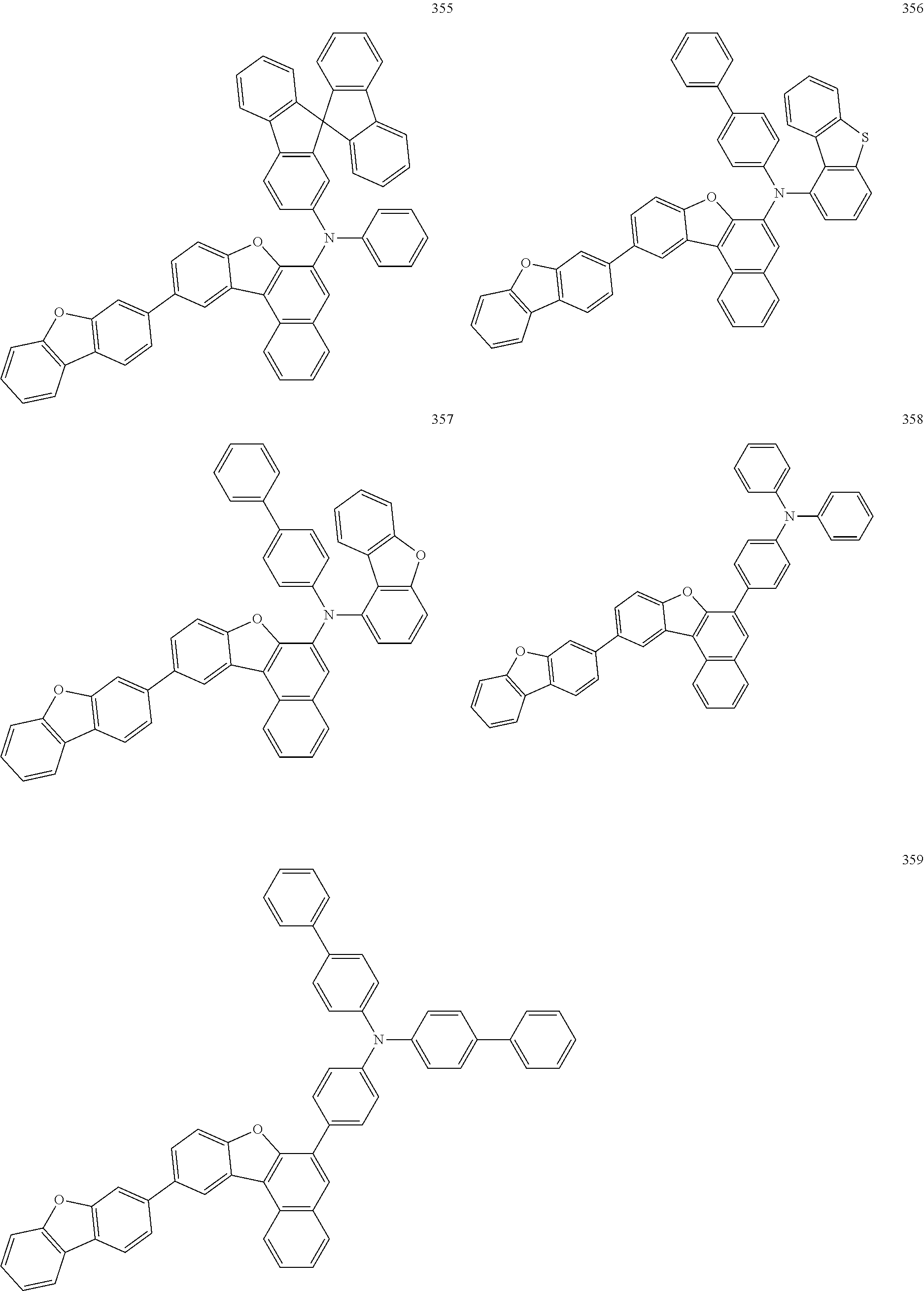 [SMILES: c1ccc(N(c2ccc3c(c2)C2(c4ccccc4-c4ccccc42)c2ccccc2-3)c2cc3ccccc3c3c2oc2ccc(-c4ccc5c(c4)oc4ccccc45)cc23)cc1 ; c1ccc(-c2ccc(N(c3cc4ccccc4c4c3oc3ccc(-c5ccc6c(c5)oc5ccccc56)cc34)c3cccc4sc5ccccc5c34)cc2)cc1 ; c1ccc(-c2ccc(N(c3cc4ccccc4c4c3oc3ccc(-c5ccc6c(c5)oc5ccccc56)cc34)c3cccc4oc5ccccc5c34)cc2)cc1 ; c1ccc(N(c2ccccc2)c2ccc(-c3cc4ccccc4c4c3oc3ccc(-c5ccc6c(c5)oc5ccccc56)cc34)cc2)cc1 ; c1ccc(-c2ccc(N(c3ccc(-c4ccccc4)cc3)c3ccc(-c4cc5ccccc5c5c4oc4ccc(-c6ccc7c(c6)oc6ccccc67)cc45)cc3)cc2)cc1]

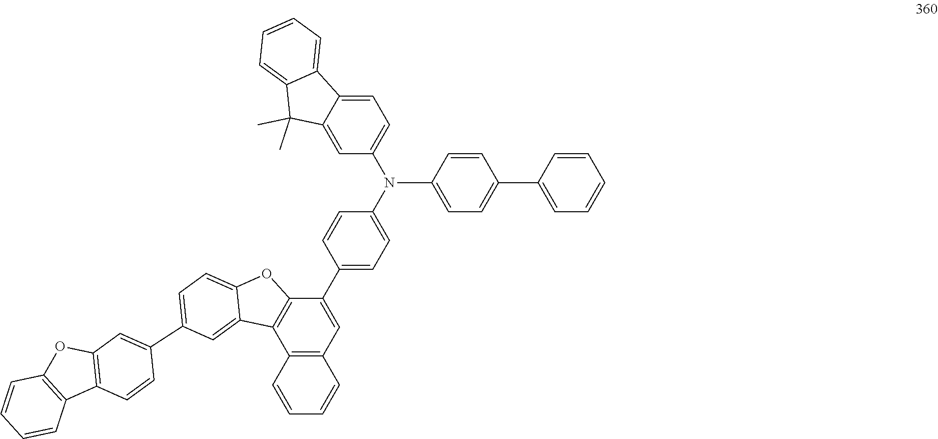 [SMILES: CC1(C)c2ccccc2-c2ccc(N(c3ccc(-c4ccccc4)cc3)c3ccc(-c4cc5ccccc5c5c4oc4ccc(-c6ccc7c(c6)oc6ccccc67)cc45)cc3)cc21]

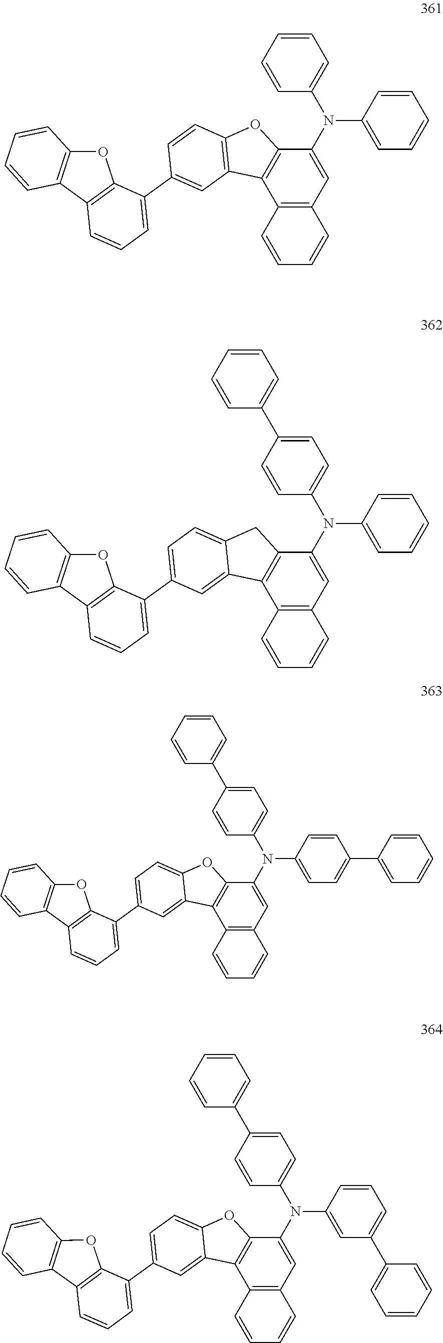 [SMILES: c1ccc(N(c2ccccc2)c2cc3ccccc3c3c2oc2ccc(-c4cccc5c4oc4ccccc45)cc23)cc1 ; c1ccc(-c2ccc(N(c3ccccc3)c3cc4ccccc4c4c3Cc3ccc(-c5cccc6c5oc5ccccc56)cc3-4)cc2)cc1 ; c1ccc(-c2ccc(N(c3ccc(-c4ccccc4)cc3)c3cc4ccccc4c4c3oc3ccc(-c5cccc6c5oc5ccccc56)cc34)cc2)cc1 ; c1ccc(-c2ccc(N(c3cccc(-c4ccccc4)c3)c3cc4ccccc4c4c3oc3ccc(-c5cccc6c5oc5ccccc56)cc34)cc2)cc1]

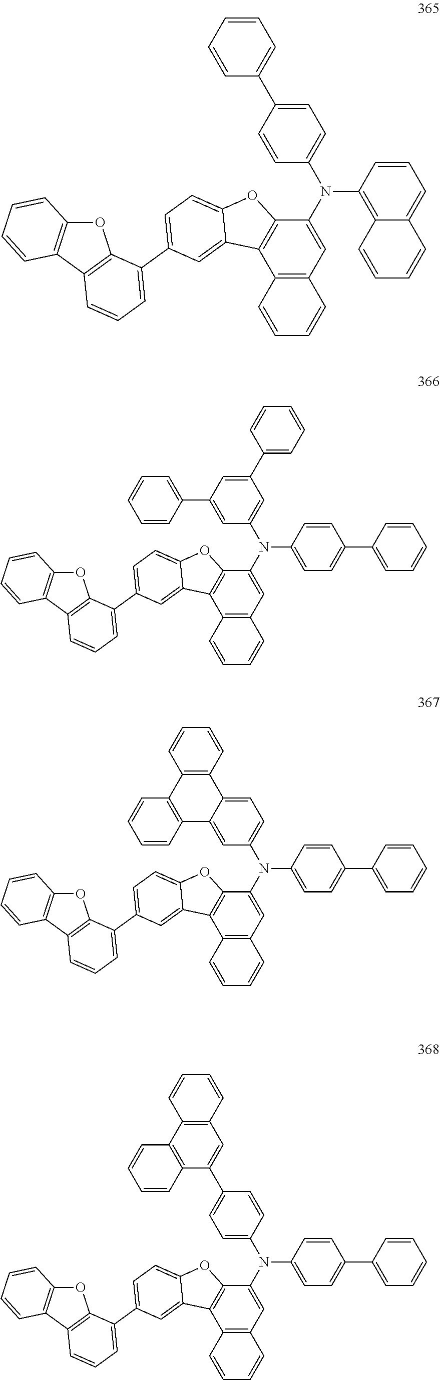 [SMILES: c1ccc(-c2ccc(N(c3cccc4ccccc34)c3cc4ccccc4c4c3oc3ccc(-c5cccc6c5oc5ccccc56)cc34)cc2)cc1 ; c1ccc(-c2ccc(N(c3cc(-c4ccccc4)cc(-c4ccccc4)c3)c3cc4ccccc4c4c3oc3ccc(-c5cccc6c5oc5ccccc56)cc34)cc2)cc1 ; c1ccc(-c2ccc(N(c3ccc4c5ccccc5c5ccccc5c4c3)c3cc4ccccc4c4c3oc3ccc(-c5cccc6c5oc5ccccc56)cc34)cc2)cc1 ; c1ccc(-c2ccc(N(c3ccc(-c4cc5ccccc5c5ccccc45)cc3)c3cc4ccccc4c4c3oc3ccc(-c5cccc6c5oc5ccccc56)cc34)cc2)cc1]

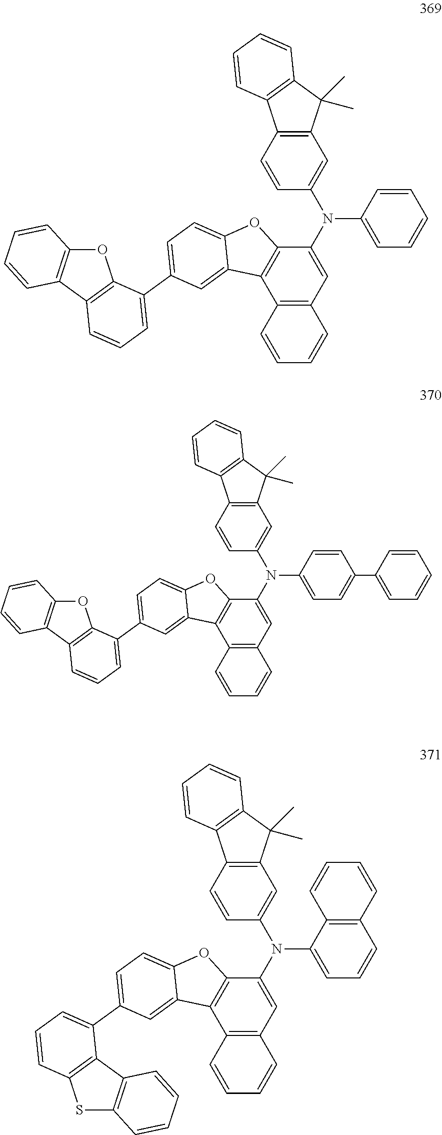 [SMILES: CC1(C)c2ccccc2-c2ccc(N(c3ccccc3)c3cc4ccccc4c4c3oc3ccc(-c5cccc6c5oc5ccccc56)cc34)cc21 ; CC1(C)c2ccccc2-c2ccc(N(c3ccc(-c4ccccc4)cc3)c3cc4ccccc4c4c3oc3ccc(-c5cccc6c5oc5ccccc56)cc34)cc21 ; CC1(C)c2ccccc2-c2ccc(N(c3cccc4ccccc34)c3cc4ccccc4c4c3oc3ccc(-c5cccc6sc7ccccc7c56)cc34)cc21]

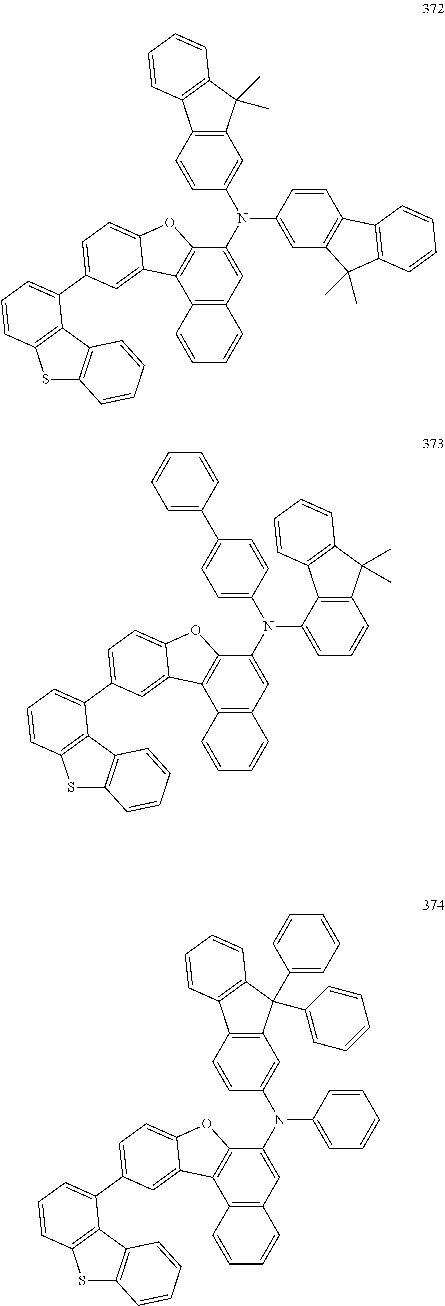 [SMILES: CC1(C)c2ccccc2-c2ccc(N(c3ccc4c(c3)C(C)(C)c3ccccc3-4)c3cc4ccccc4c4c3oc3ccc(-c5cccc6sc7ccccc7c56)cc34)cc21 ; CC1(C)c2ccccc2-c2c(N(c3ccc(-c4ccccc4)cc3)c3cc4ccccc4c4c3oc3ccc(-c5cccc6sc7ccccc7c56)cc34)cccc21 ; c1ccc(N(c2ccc3c(c2)C(c2ccccc2)(c2ccccc2)c2ccccc2-3)c2cc3ccccc3c3c2oc2ccc(-c4cccc5sc6ccccc6c45)cc23)cc1]

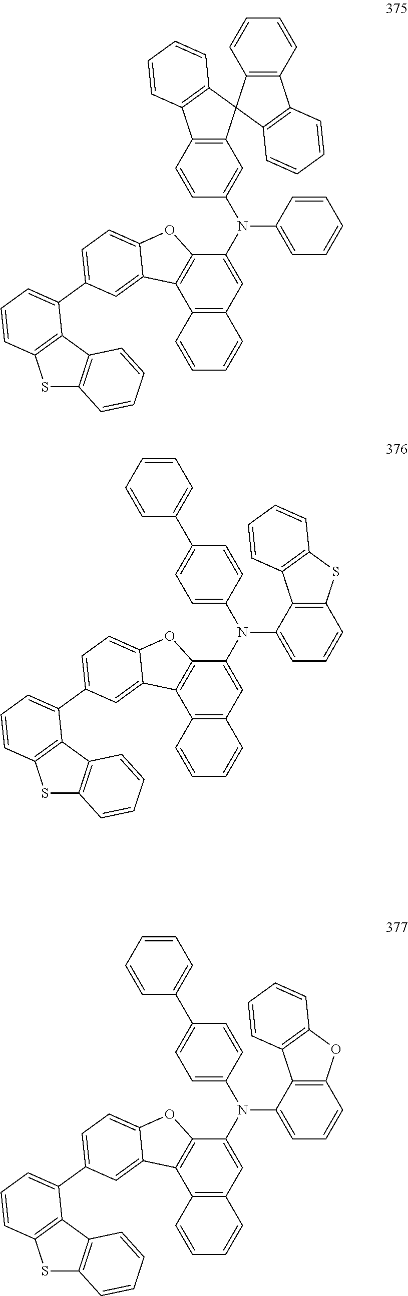 [SMILES: c1ccc(N(c2ccc3c(c2)C2(c4ccccc4-c4ccccc42)c2ccccc2-3)c2cc3ccccc3c3c2oc2ccc(-c4cccc5sc6ccccc6c45)cc23)cc1 ; c1ccc(-c2ccc(N(c3cc4ccccc4c4c3oc3ccc(-c5cccc6sc7ccccc7c56)cc34)c3cccc4sc5ccccc5c34)cc2)cc1 ; c1ccc(-c2ccc(N(c3cc4ccccc4c4c3oc3ccc(-c5cccc6sc7ccccc7c56)cc34)c3cccc4oc5ccccc5c34)cc2)cc1]

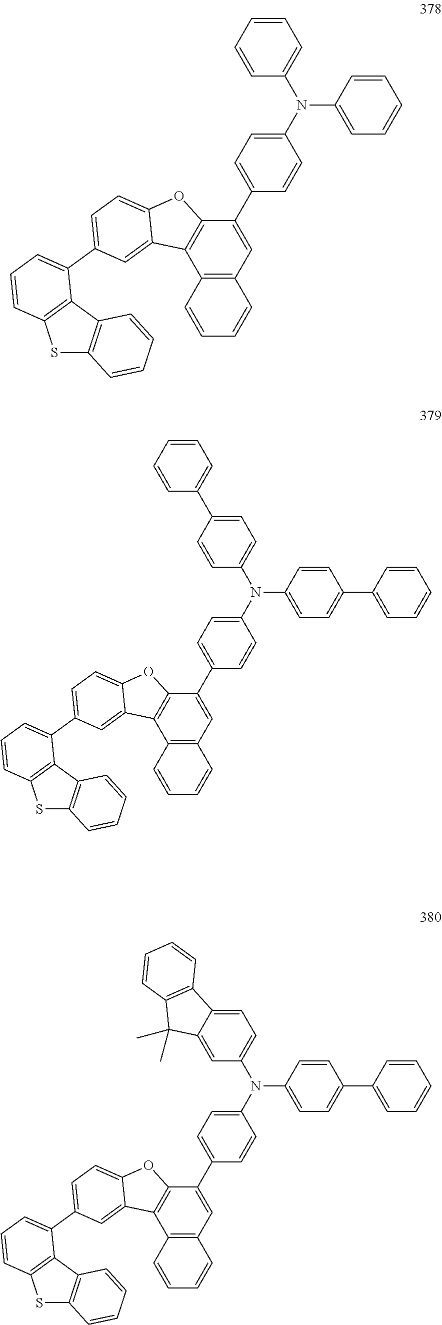 [SMILES: c1ccc(N(c2ccccc2)c2ccc(-c3cc4ccccc4c4c3oc3ccc(-c5cccc6sc7ccccc7c56)cc34)cc2)cc1 ; c1ccc(-c2ccc(N(c3ccc(-c4ccccc4)cc3)c3ccc(-c4cc5ccccc5c5c4oc4ccc(-c6cccc7sc8ccccc8c67)cc45)cc3)cc2)cc1 ; CC1(C)c2ccccc2-c2ccc(N(c3ccc(-c4ccccc4)cc3)c3ccc(-c4cc5ccccc5c5c4oc4ccc(-c6cccc7sc8ccccc8c67)cc45)cc3)cc21]

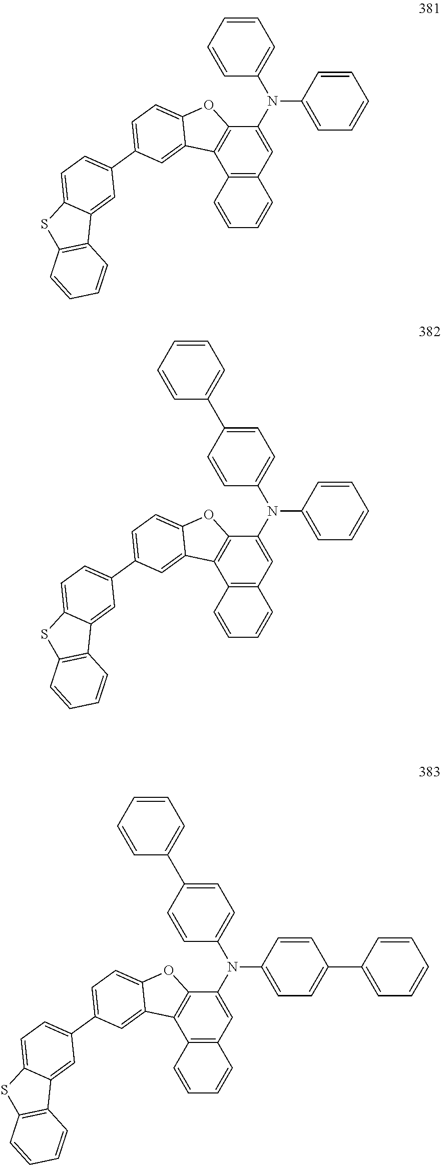 [SMILES: c1ccc(N(c2ccccc2)c2cc3ccccc3c3c2oc2ccc(-c4ccc5sc6ccccc6c5c4)cc23)cc1 ; c1ccc(-c2ccc(N(c3ccccc3)c3cc4ccccc4c4c3oc3ccc(-c5ccc6sc7ccccc7c6c5)cc34)cc2)cc1 ; c1ccc(-c2ccc(N(c3ccc(-c4ccccc4)cc3)c3cc4ccccc4c4c3oc3ccc(-c5ccc6sc7ccccc7c6c5)cc34)cc2)cc1]

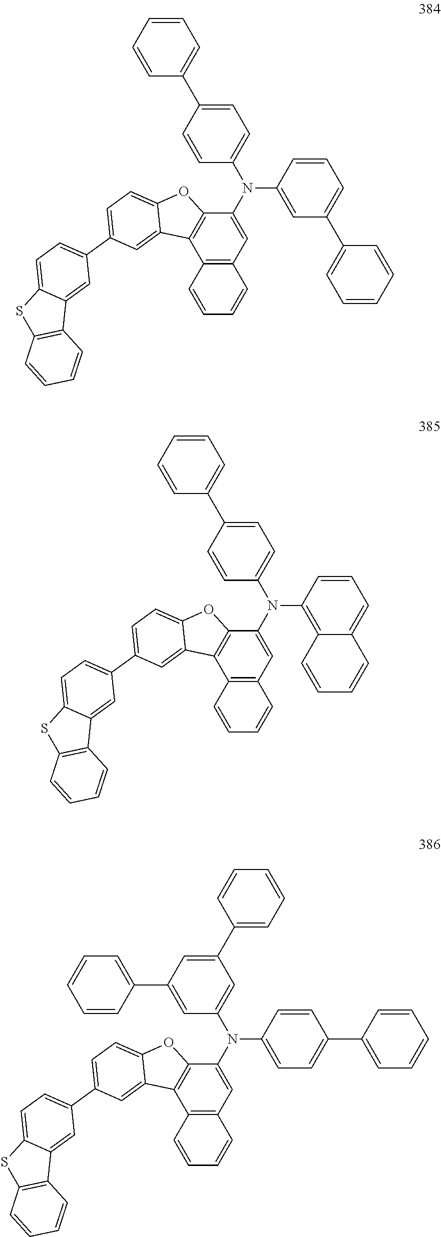 [SMILES: c1ccc(-c2ccc(N(c3cccc(-c4ccccc4)c3)c3cc4ccccc4c4c3oc3ccc(-c5ccc6sc7ccccc7c6c5)cc34)cc2)cc1 ; c1ccc(-c2ccc(N(c3cccc4ccccc34)c3cc4ccccc4c4c3oc3ccc(-c5ccc6sc7ccccc7c6c5)cc34)cc2)cc1 ; c1ccc(-c2ccc(N(c3cc(-c4ccccc4)cc(-c4ccccc4)c3)c3cc4ccccc4c4c3oc3ccc(-c5ccc6sc7ccccc7c6c5)cc34)cc2)cc1]

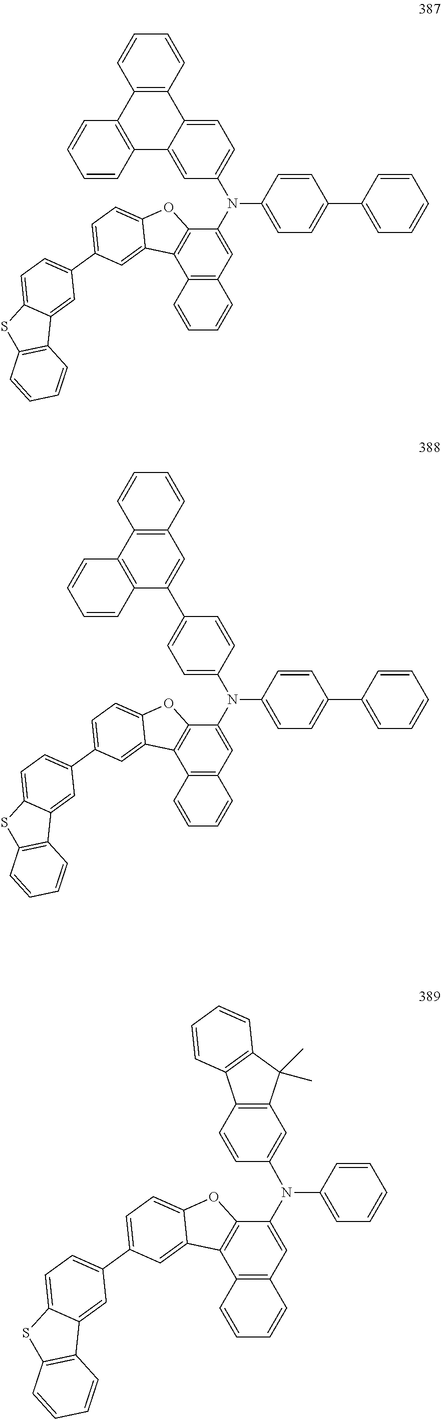 [SMILES: c1ccc(-c2ccc(N(c3ccc4c5ccccc5c5ccccc5c4c3)c3cc4ccccc4c4c3oc3ccc(-c5ccc6sc7ccccc7c6c5)cc34)cc2)cc1 ; c1ccc(-c2ccc(N(c3ccc(-c4cc5ccccc5c5ccccc45)cc3)c3cc4ccccc4c4c3oc3ccc(-c5ccc6sc7ccccc7c6c5)cc34)cc2)cc1 ; CC1(C)c2ccccc2-c2ccc(N(c3ccccc3)c3cc4ccccc4c4c3oc3ccc(-c5ccc6sc7ccccc7c6c5)cc34)cc21]

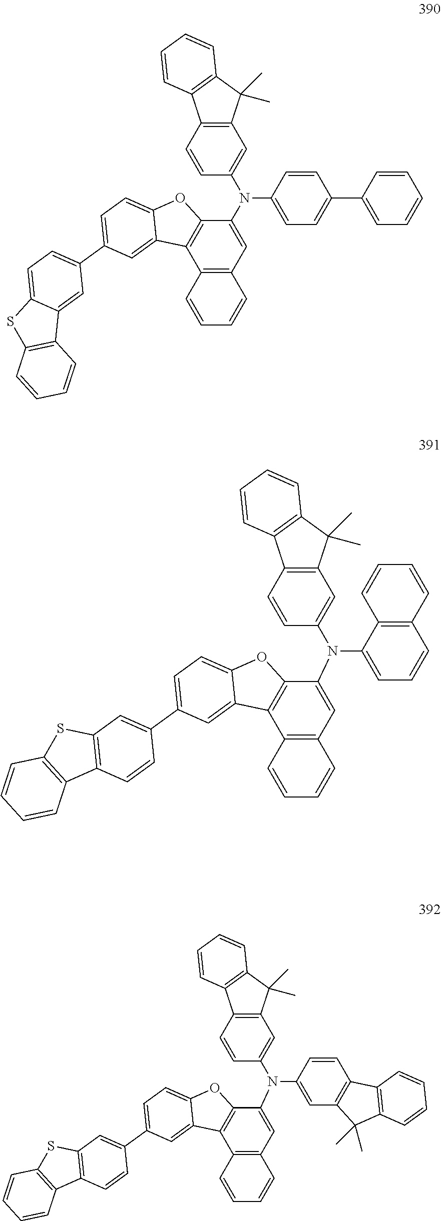 [SMILES: CC1(C)c2ccccc2-c2ccc(N(c3ccc(-c4ccccc4)cc3)c3cc4ccccc4c4c3oc3ccc(-c5ccc6sc7ccccc7c6c5)cc34)cc21 ; CC1(C)c2ccccc2-c2ccc(N(c3cccc4ccccc34)c3cc4ccccc4c4c3oc3ccc(-c5ccc6c(c5)sc5ccccc56)cc34)cc21 ; CC1(C)c2ccccc2-c2ccc(N(c3ccc4c(c3)C(C)(C)c3ccccc3-4)c3cc4ccccc4c4c3oc3ccc(-c5ccc6c(c5)sc5ccccc56)cc34)cc21]

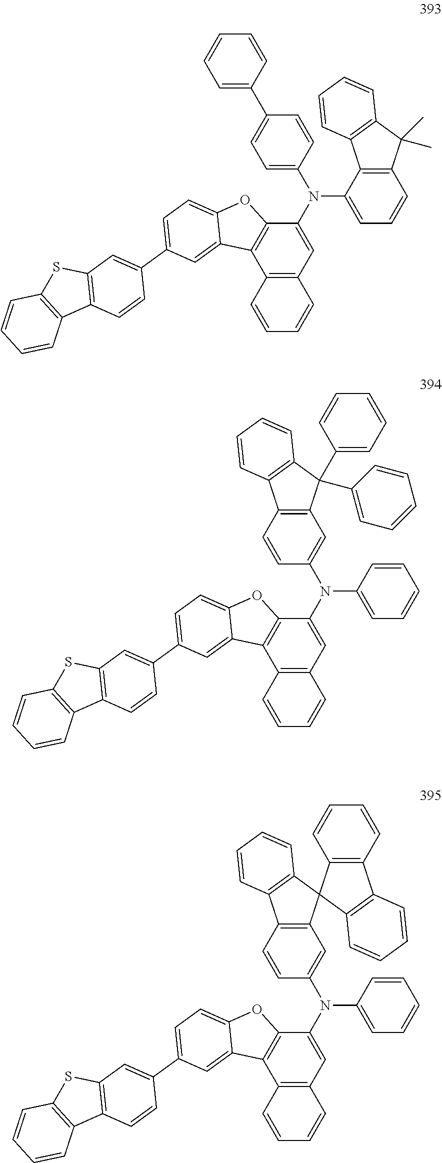 [SMILES: CC1(C)c2ccccc2-c2c(N(c3ccc(-c4ccccc4)cc3)c3cc4ccccc4c4c3oc3ccc(-c5ccc6c(c5)sc5ccccc56)cc34)cccc21 ; c1ccc(N(c2ccc3c(c2)C(c2ccccc2)(c2ccccc2)c2ccccc2-3)c2cc3ccccc3c3c2oc2ccc(-c4ccc5c(c4)sc4ccccc45)cc23)cc1 ; c1ccc(N(c2ccc3c(c2)C2(c4ccccc4-c4ccccc42)c2ccccc2-3)c2cc3ccccc3c3c2oc2ccc(-c4ccc5c(c4)sc4ccccc45)cc23)cc1]

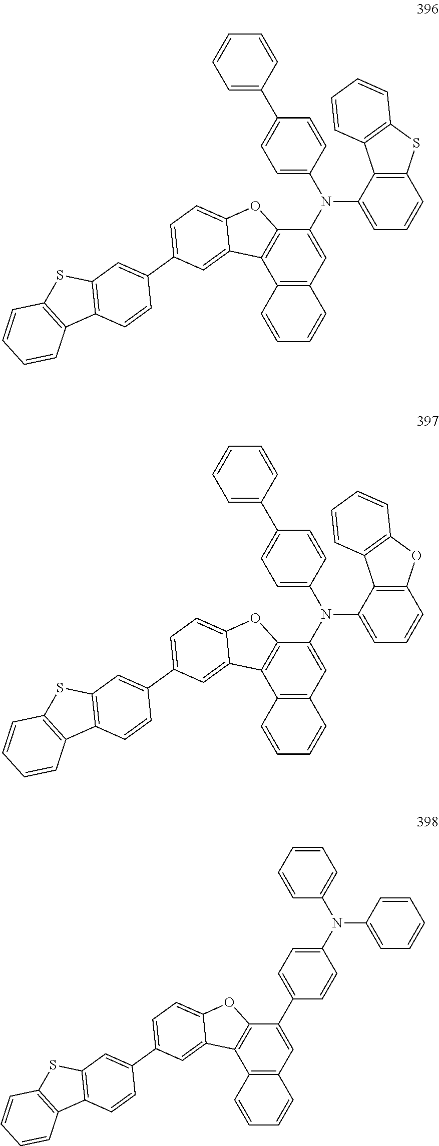 [SMILES: c1ccc(-c2ccc(N(c3cc4ccccc4c4c3oc3ccc(-c5ccc6c(c5)sc5ccccc56)cc34)c3cccc4sc5ccccc5c34)cc2)cc1 ; c1ccc(-c2ccc(N(c3cc4ccccc4c4c3oc3ccc(-c5ccc6c(c5)sc5ccccc56)cc34)c3cccc4oc5ccccc5c34)cc2)cc1 ; c1ccc(N(c2ccccc2)c2ccc(-c3cc4ccccc4c4c3oc3ccc(-c5ccc6c(c5)sc5ccccc56)cc34)cc2)cc1]

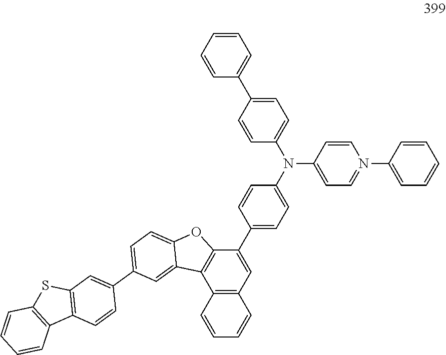

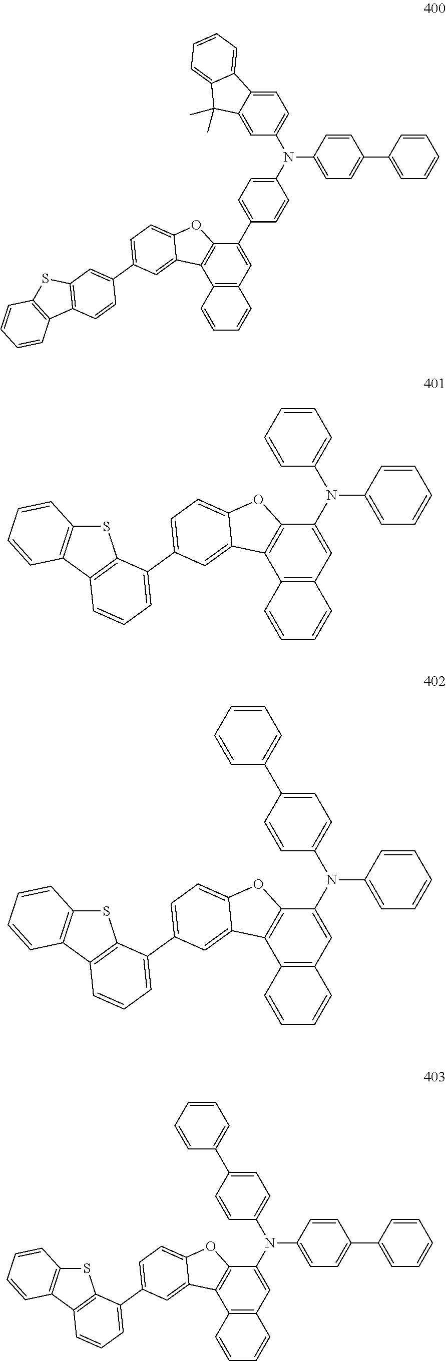 [SMILES: CC1(C)c2ccccc2-c2ccc(N(c3ccc(-c4ccccc4)cc3)c3ccc(-c4cc5ccccc5c5c4oc4ccc(-c6ccc7c(c6)sc6ccccc67)cc45)cc3)cc21 ; c1ccc(N(c2ccccc2)c2cc3ccccc3c3c2oc2ccc(-c4cccc5c4sc4ccccc45)cc23)cc1 ; c1ccc(-c2ccc(N(c3ccccc3)c3cc4ccccc4c4c3oc3ccc(-c5cccc6c5sc5ccccc56)cc34)cc2)cc1 ; c1ccc(-c2ccc(N(c3ccc(-c4ccccc4)cc3)c3cc4ccccc4c4c3oc3ccc(-c5cccc6c5sc5ccccc56)cc34)cc2)cc1]

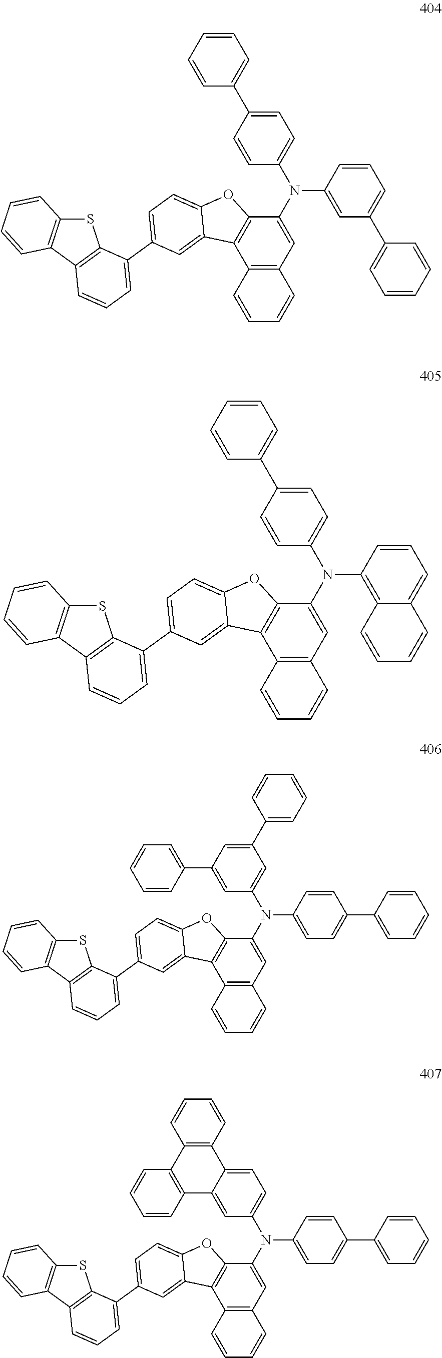 [SMILES: c1ccc(-c2ccc(N(c3cccc(-c4ccccc4)c3)c3cc4ccccc4c4c3oc3ccc(-c5cccc6c5sc5ccccc56)cc34)cc2)cc1 ; c1ccc(-c2ccc(N(c3cccc4ccccc34)c3cc4ccccc4c4c3oc3ccc(-c5cccc6c5sc5ccccc56)cc34)cc2)cc1 ; c1ccc(-c2ccc(N(c3cc(-c4ccccc4)cc(-c4ccccc4)c3)c3cc4ccccc4c4c3oc3ccc(-c5cccc6c5sc5ccccc56)cc34)cc2)cc1 ; c1ccc(-c2ccc(N(c3ccc4c5ccccc5c5ccccc5c4c3)c3cc4ccccc4c4c3oc3ccc(-c5cccc6c5sc5ccccc56)cc34)cc2)cc1]

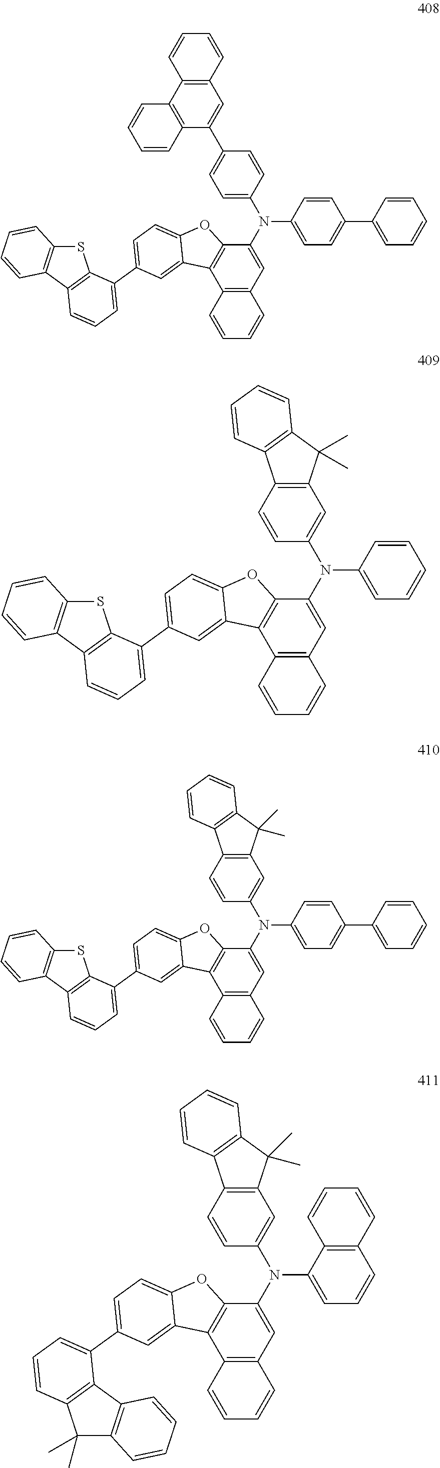 [SMILES: c1ccc(-c2ccc(N(c3ccc(-c4cc5ccccc5c5ccccc45)cc3)c3cc4ccccc4c4c3oc3ccc(-c5cccc6c5sc5ccccc56)cc34)cc2)cc1 ; CC1(C)c2ccccc2-c2ccc(N(c3ccccc3)c3cc4ccccc4c4c3oc3ccc(-c5cccc6c5sc5ccccc56)cc34)cc21 ; CC1(C)c2ccccc2-c2ccc(N(c3ccc(-c4ccccc4)cc3)c3cc4ccccc4c4c3oc3ccc(-c5cccc6c5sc5ccccc56)cc34)cc21 ; CC1(C)c2ccccc2-c2ccc(N(c3cccc4ccccc34)c3cc4ccccc4c4c3oc3ccc(-c5cccc6c5-c5ccccc5C6(C)C)cc34)cc21]

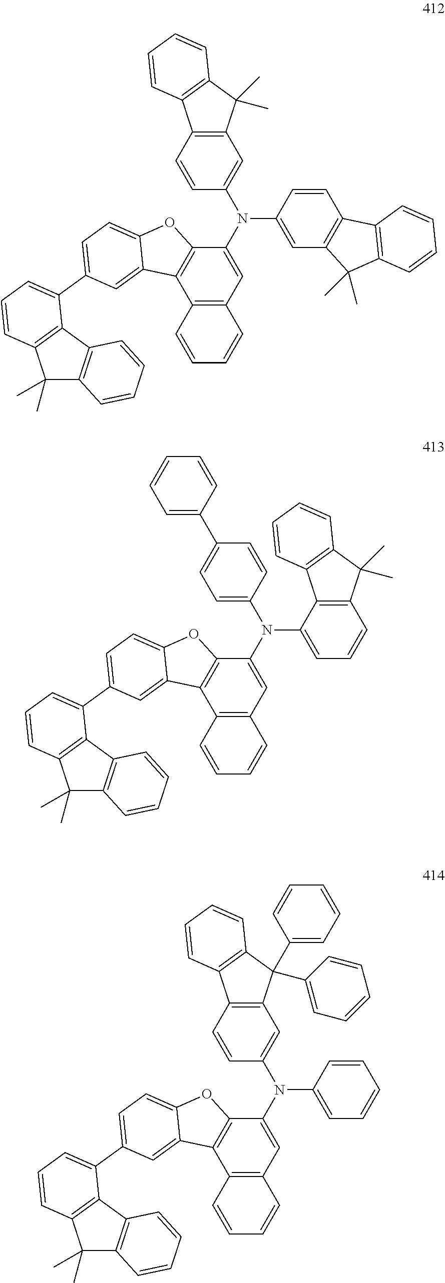 [SMILES: CC1(C)c2ccccc2-c2ccc(N(c3ccc4c(c3)C(C)(C)c3ccccc3-4)c3cc4ccccc4c4c3oc3ccc(-c5cccc6c5-c5ccccc5C6(C)C)cc34)cc21 ; CC1(C)c2ccccc2-c2c(-c3ccc4oc5c(N(c6ccc(-c7ccccc7)cc6)c6cccc7c6-c6ccccc6C7(C)C)cc6ccccc6c5c4c3)cccc21 ; CC1(C)c2ccccc2-c2c(-c3ccc4oc5c(N(c6ccccc6)c6ccc7c(c6)C(c6ccccc6)(c6ccccc6)c6ccccc6-7)cc6ccccc6c5c4c3)cccc21]

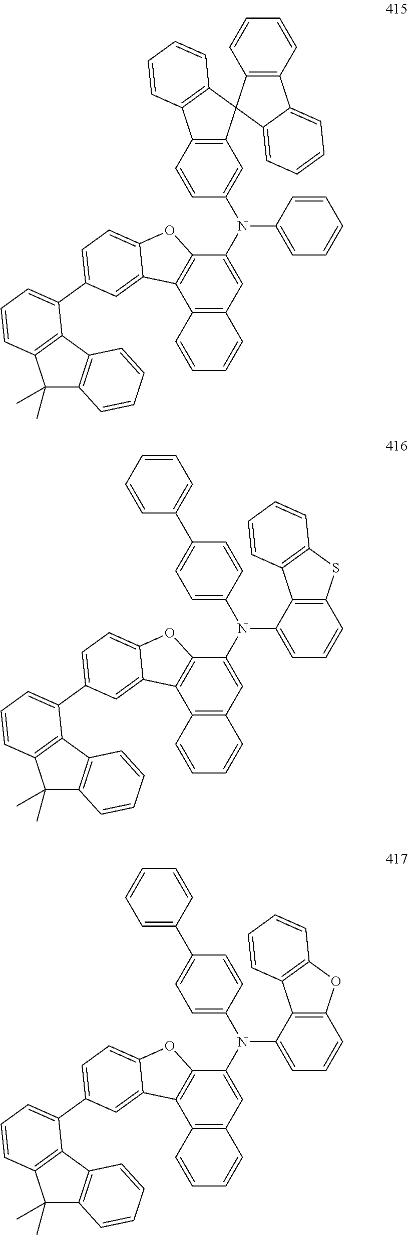 [SMILES: CC1(C)c2ccccc2-c2c(-c3ccc4oc5c(N(c6ccccc6)c6ccc7c(c6)C6(c8ccccc8-c8ccccc86)c6ccccc6-7)cc6ccccc6c5c4c3)cccc21 ; CC1(C)c2ccccc2-c2c(-c3ccc4oc5c(N(c6ccc(-c7ccccc7)cc6)c6cccc7sc8ccccc8c67)cc6ccccc6c5c4c3)cccc21 ; CC1(C)c2ccccc2-c2c(-c3ccc4oc5c(N(c6ccc(-c7ccccc7)cc6)c6cccc7oc8ccccc8c67)cc6ccccc6c5c4c3)cccc21]

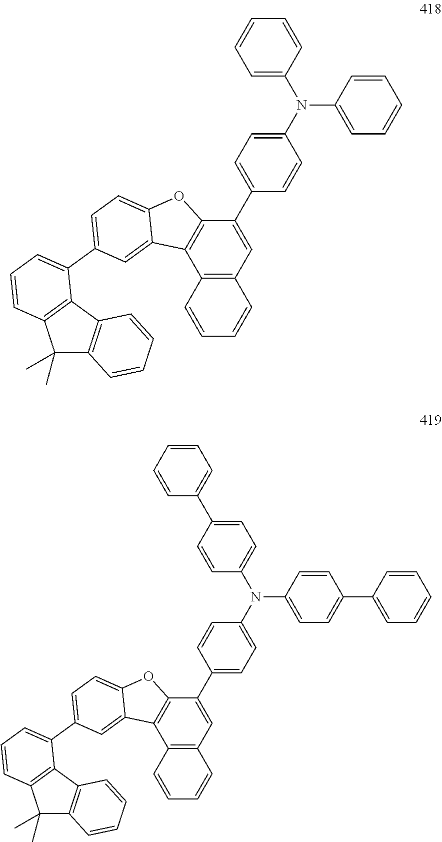 [SMILES: CC1(C)c2ccccc2-c2c(-c3ccc4oc5c(-c6ccc(N(c7ccccc7)c7ccccc7)cc6)cc6ccccc6c5c4c3)cccc21 ; CC1(C)c2ccccc2-c2c(-c3ccc4oc5c(-c6ccc(N(c7ccc(-c8ccccc8)cc7)c7ccc(-c8ccccc8)cc7)cc6)cc6ccccc6c5c4c3)cccc21]

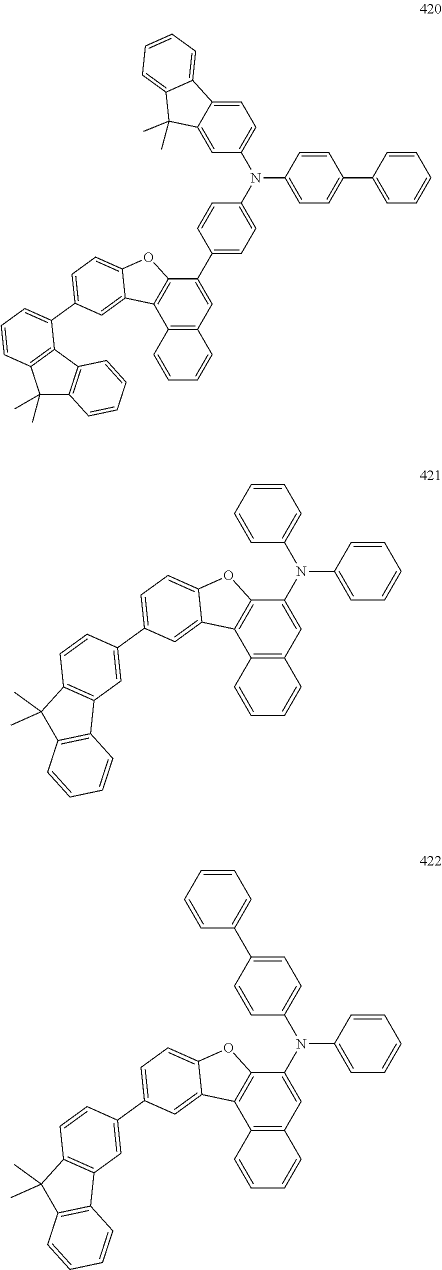 [SMILES: CC1(C)c2ccccc2-c2ccc(N(c3ccc(-c4ccccc4)cc3)c3ccc(-c4cc5ccccc5c5c4oc4ccc(-c6cccc7c6-c6ccccc6C7(C)C)cc45)cc3)cc21 ; CC1(C)c2ccccc2-c2cc(-c3ccc4oc5c(N(c6ccccc6)c6ccccc6)cc6ccccc6c5c4c3)ccc21 ; CC1(C)c2ccccc2-c2cc(-c3ccc4oc5c(N(c6ccccc6)c6ccc(-c7ccccc7)cc6)cc6ccccc6c5c4c3)ccc21]

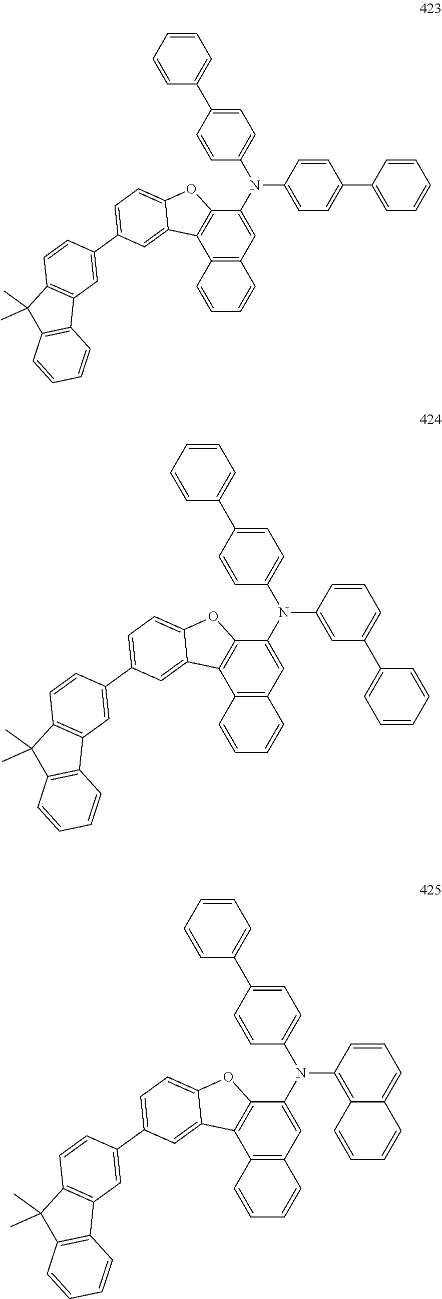 [SMILES: CC1(C)c2ccccc2-c2cc(-c3ccc4oc5c(N(c6ccc(-c7ccccc7)cc6)c6ccc(-c7ccccc7)cc6)cc6ccccc6c5c4c3)ccc21 ; CC1(C)c2ccccc2-c2cc(-c3ccc4oc5c(N(c6ccc(-c7ccccc7)cc6)c6cccc(-c7ccccc7)c6)cc6ccccc6c5c4c3)ccc21 ; CC1(C)c2ccccc2-c2cc(-c3ccc4oc5c(N(c6ccc(-c7ccccc7)cc6)c6cccc7ccccc67)cc6ccccc6c5c4c3)ccc21]

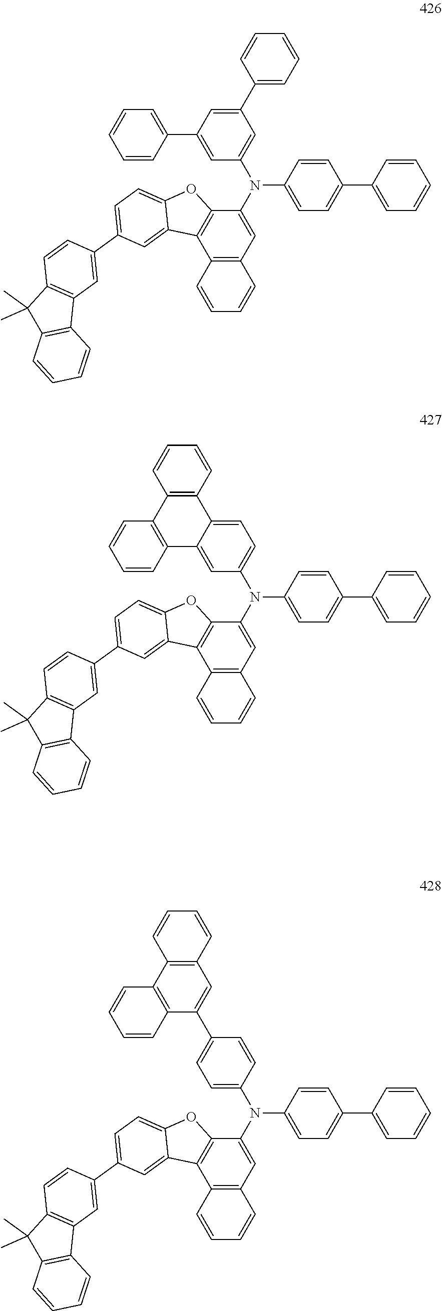 [SMILES: CC1(C)c2ccccc2-c2cc(-c3ccc4oc5c(N(c6ccc(-c7ccccc7)cc6)c6cc(-c7ccccc7)cc(-c7ccccc7)c6)cc6ccccc6c5c4c3)ccc21 ; CC1(C)c2ccccc2-c2cc(-c3ccc4oc5c(N(c6ccc(-c7ccccc7)cc6)c6ccc7c8ccccc8c8ccccc8c7c6)cc6ccccc6c5c4c3)ccc21 ; CC1(C)c2ccccc2-c2cc(-c3ccc4oc5c(N(c6ccc(-c7ccccc7)cc6)c6ccc(-c7cc8ccccc8c8ccccc78)cc6)cc6ccccc6c5c4c3)ccc21]

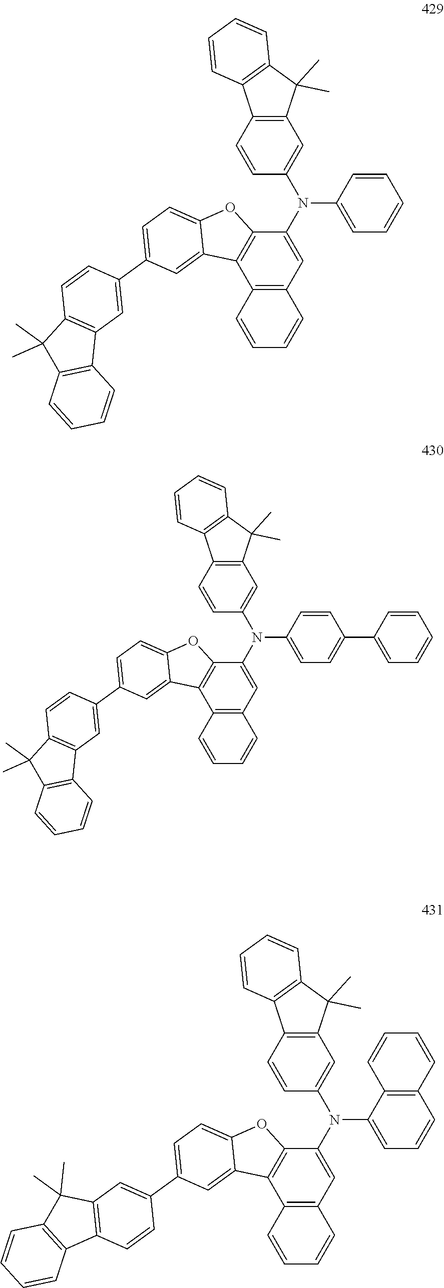 [SMILES: CC1(C)c2ccccc2-c2cc(-c3ccc4oc5c(N(c6ccccc6)c6ccc7c(c6)C(C)(C)c6ccccc6-7)cc6ccccc6c5c4c3)ccc21 ; CC1(C)c2ccccc2-c2cc(-c3ccc4oc5c(N(c6ccc(-c7ccccc7)cc6)c6ccc7c(c6)C(C)(C)c6ccccc6-7)cc6ccccc6c5c4c3)ccc21 ; CC1(C)c2ccccc2-c2ccc(-c3ccc4oc5c(N(c6ccc7c(c6)C(C)(C)c6ccccc6-7)c6cccc7ccccc67)cc6ccccc6c5c4c3)cc21]

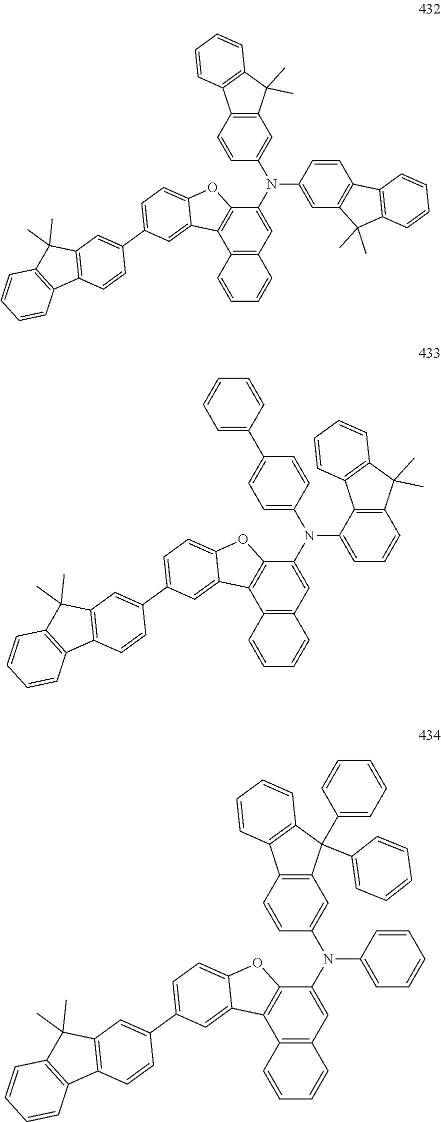 [SMILES: CC1(C)c2ccccc2-c2ccc(-c3ccc4oc5c(N(c6ccc7c(c6)C(C)(C)c6ccccc6-7)c6ccc7c(c6)C(C)(C)c6ccccc6-7)cc6ccccc6c5c4c3)cc21 ; CC1(C)c2ccccc2-c2ccc(-c3ccc4oc5c(N(c6ccc(-c7ccccc7)cc6)c6cccc7c6-c6ccccc6C7(C)C)cc6ccccc6c5c4c3)cc21 ; CC1(C)c2ccccc2-c2ccc(-c3ccc4oc5c(N(c6ccccc6)c6ccc7c(c6)C(c6ccccc6)(c6ccccc6)c6ccccc6-7)cc6ccccc6c5c4c3)cc21]

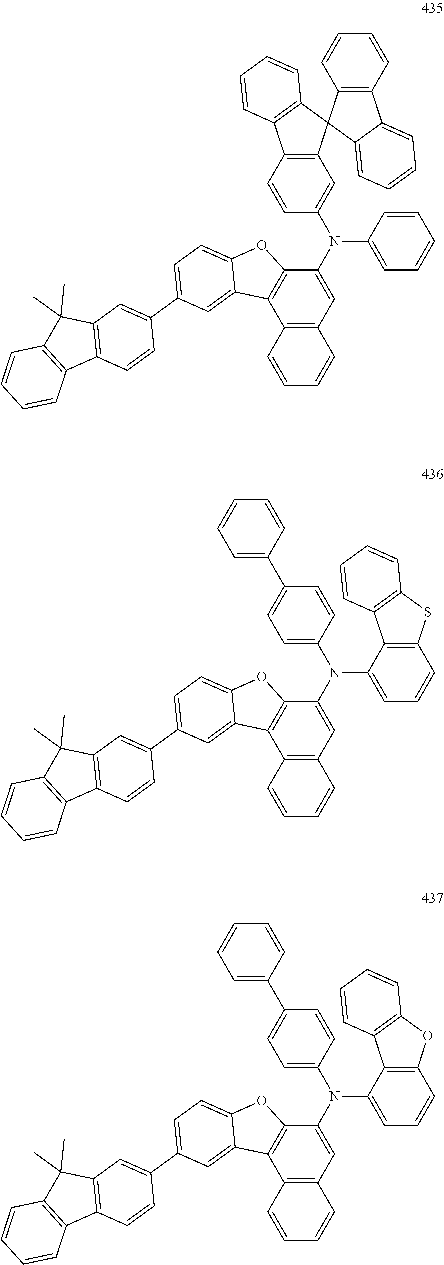 [SMILES: CC1(C)c2ccccc2-c2ccc(-c3ccc4oc5c(N(c6ccccc6)c6ccc7c(c6)C6(c8ccccc8-c8ccccc86)c6ccccc6-7)cc6ccccc6c5c4c3)cc21 ; CC1(C)c2ccccc2-c2ccc(-c3ccc4oc5c(N(c6ccc(-c7ccccc7)cc6)c6cccc7sc8ccccc8c67)cc6ccccc6c5c4c3)cc21 ; CC1(C)c2ccccc2-c2ccc(-c3ccc4oc5c(N(c6ccc(-c7ccccc7)cc6)c6cccc7oc8ccccc8c67)cc6ccccc6c5c4c3)cc21]

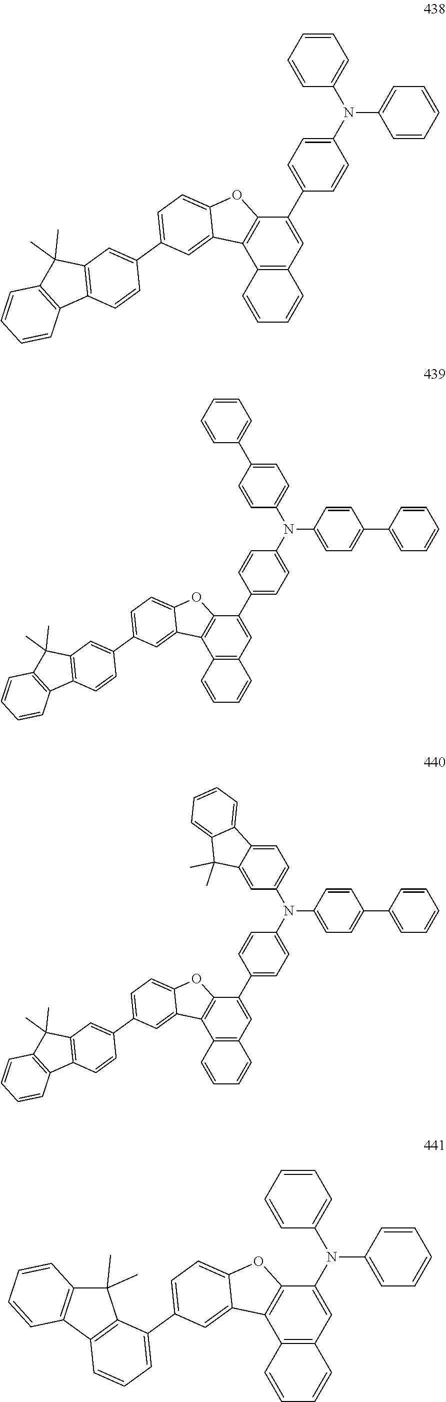 [SMILES: CC1(C)c2ccccc2-c2ccc(-c3ccc4oc5c(-c6ccc(N(c7ccccc7)c7ccccc7)cc6)cc6ccccc6c5c4c3)cc21 ; CC1(C)c2ccccc2-c2ccc(-c3ccc4oc5c(-c6ccc(N(c7ccc(-c8ccccc8)cc7)c7ccc(-c8ccccc8)cc7)cc6)cc6ccccc6c5c4c3)cc21 ; CC1(C)c2ccccc2-c2ccc(-c3ccc4oc5c(-c6ccc(N(c7ccc(-c8ccccc8)cc7)c7ccc8c(c7)C(C)(C)c7ccccc7-8)cc6)cc6ccccc6c5c4c3)cc21 ; CC1(C)c2ccccc2-c2cccc(-c3ccc4oc5c(N(c6ccccc6)c6ccccc6)cc6ccccc6c5c4c3)c21]

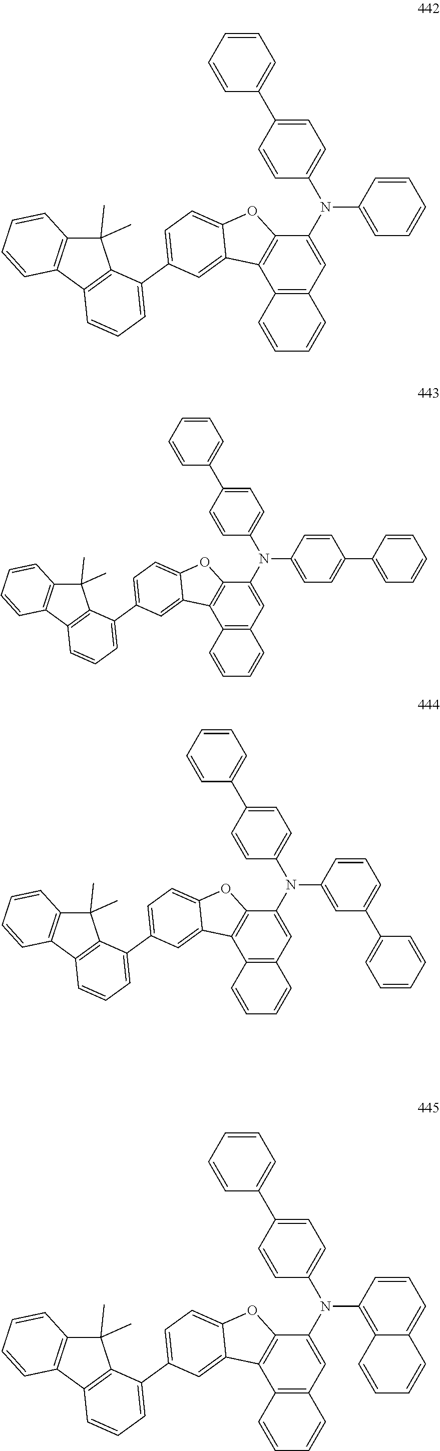 [SMILES: CC1(C)c2ccccc2-c2cccc(-c3ccc4oc5c(N(c6ccccc6)c6ccc(-c7ccccc7)cc6)cc6ccccc6c5c4c3)c21 ; CC1(C)c2ccccc2-c2cccc(-c3ccc4oc5c(N(c6ccc(-c7ccccc7)cc6)c6ccc(-c7ccccc7)cc6)cc6ccccc6c5c4c3)c21 ; CC1(C)c2ccccc2-c2cccc(-c3ccc4oc5c(N(c6ccc(-c7ccccc7)cc6)c6cccc(-c7ccccc7)c6)cc6ccccc6c5c4c3)c21 ; CC1(C)c2ccccc2-c2cccc(-c3ccc4oc5c(N(c6ccc(-c7ccccc7)cc6)c6cccc7ccccc67)cc6ccccc6c5c4c3)c21]

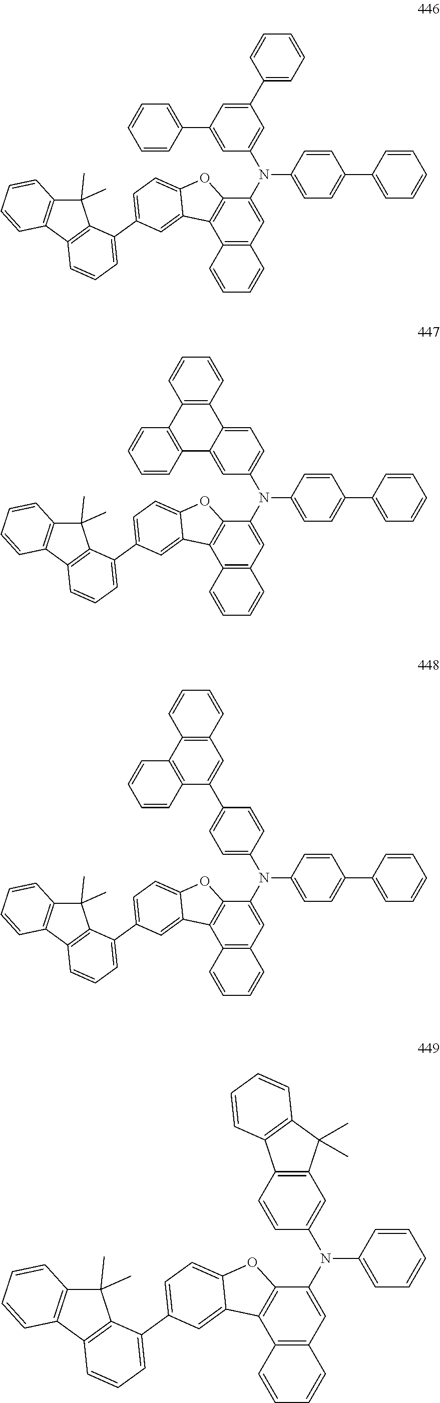 [SMILES: CC1(C)c2ccccc2-c2cccc(-c3ccc4oc5c(N(c6ccc(-c7ccccc7)cc6)c6cc(-c7ccccc7)cc(-c7ccccc7)c6)cc6ccccc6c5c4c3)c21 ; CC1(C)c2ccccc2-c2cccc(-c3ccc4oc5c(N(c6ccc(-c7ccccc7)cc6)c6ccc7c8ccccc8c8ccccc8c7c6)cc6ccccc6c5c4c3)c21 ; CC1(C)c2ccccc2-c2cccc(-c3ccc4oc5c(N(c6ccc(-c7ccccc7)cc6)c6ccc(-c7cc8ccccc8c8ccccc78)cc6)cc6ccccc6c5c4c3)c21 ; CC1(C)c2ccccc2-c2ccc(N(c3ccccc3)c3cc4ccccc4c4c3oc3ccc(-c5cccc6c5C(C)(C)c5ccccc5-6)cc34)cc21]

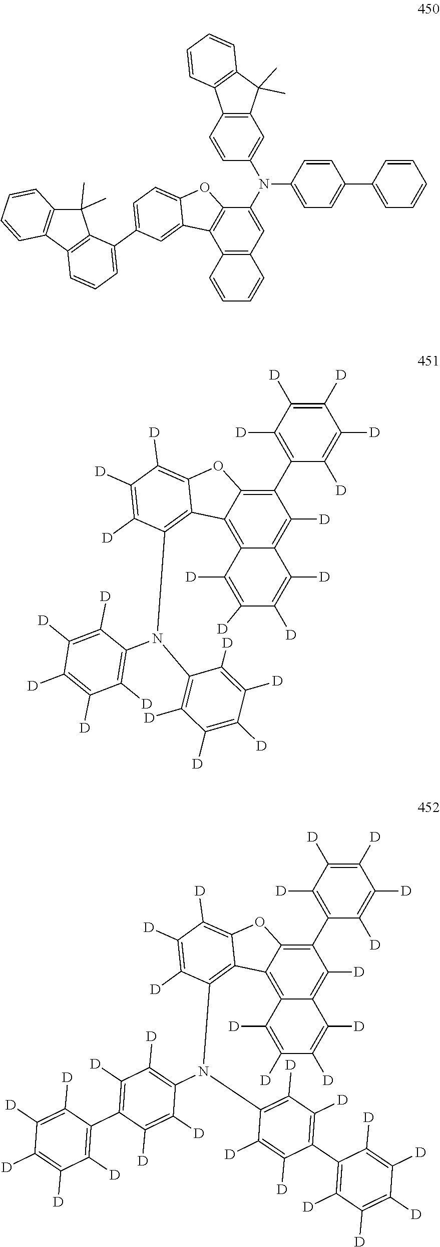

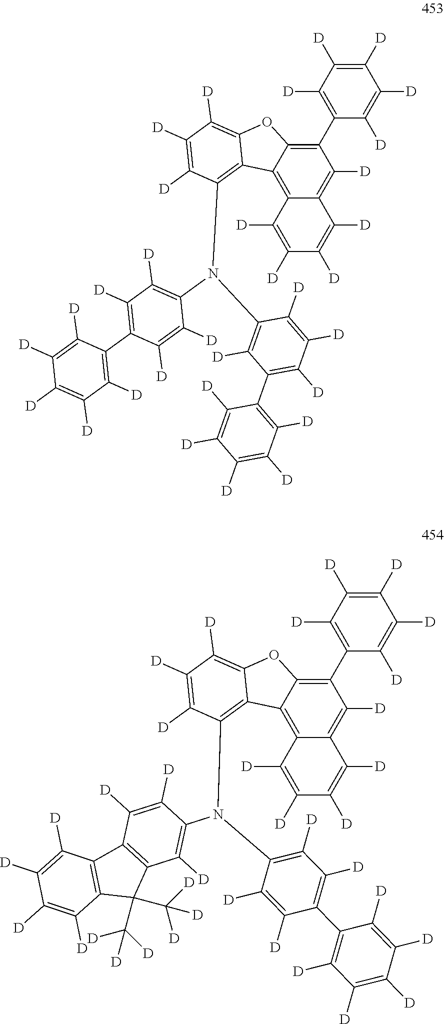

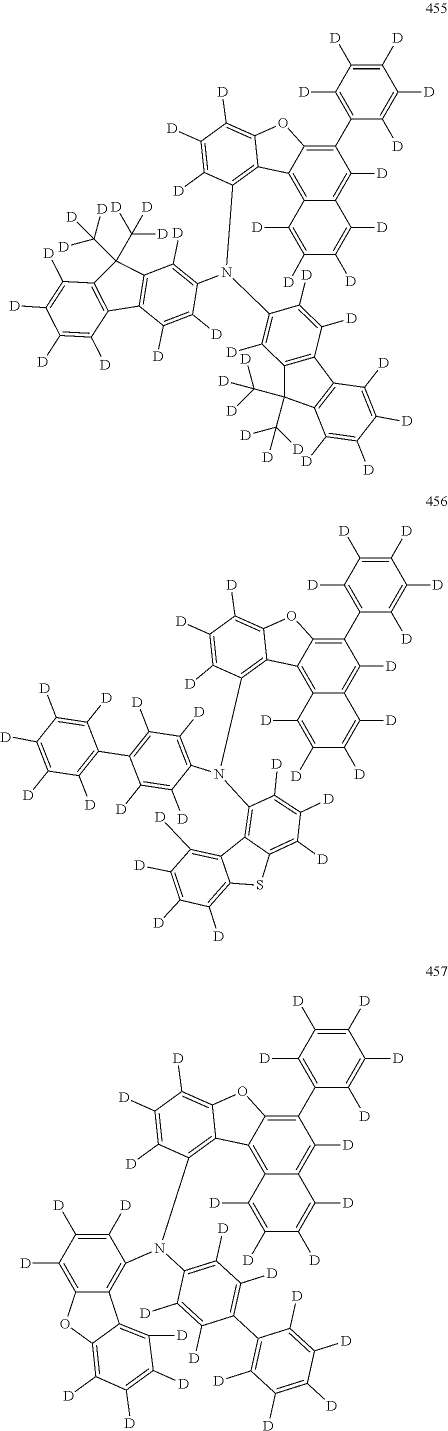

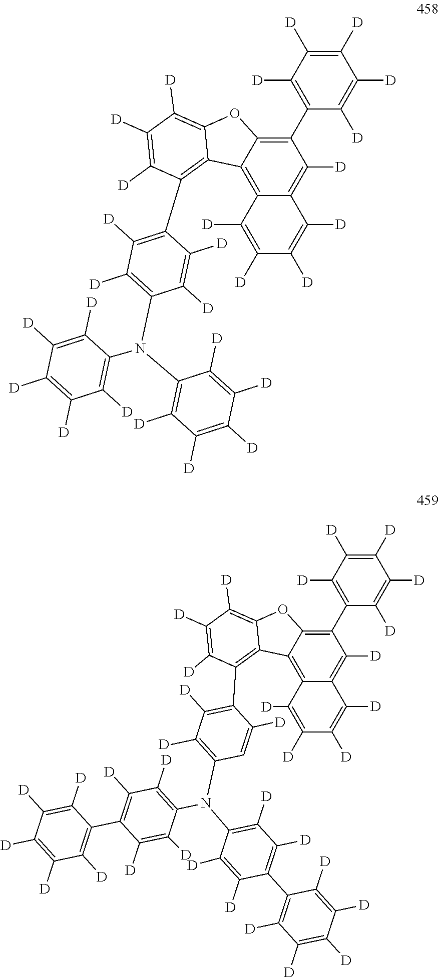

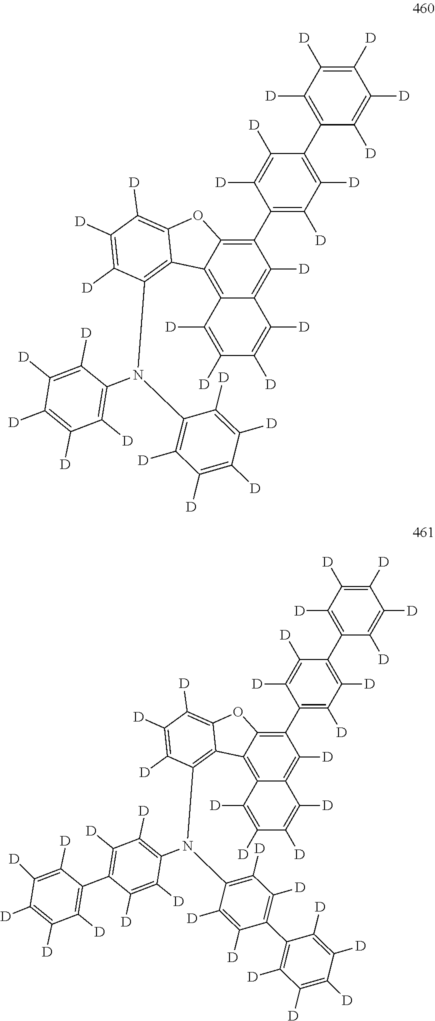

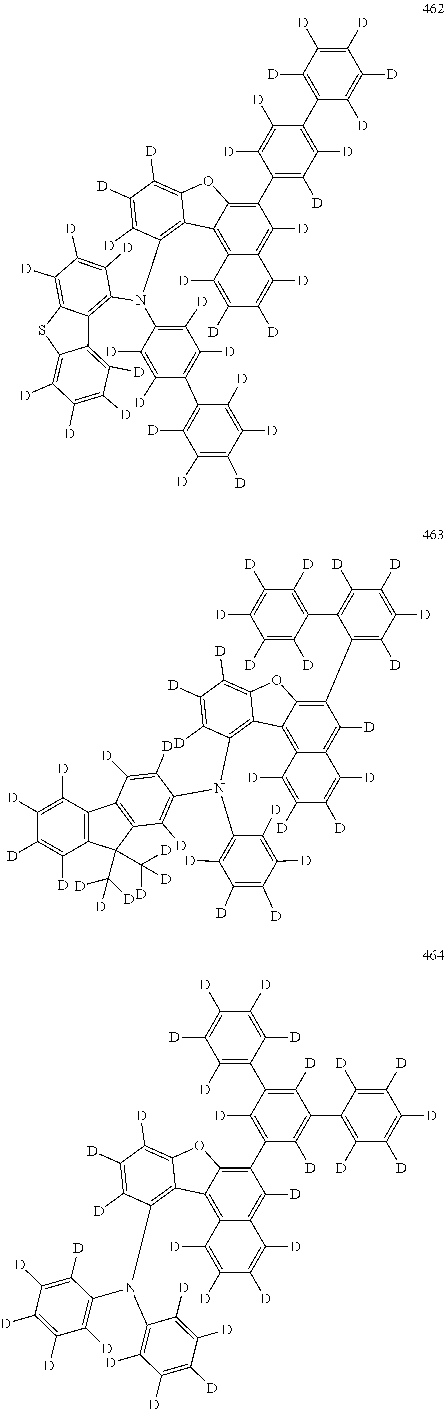

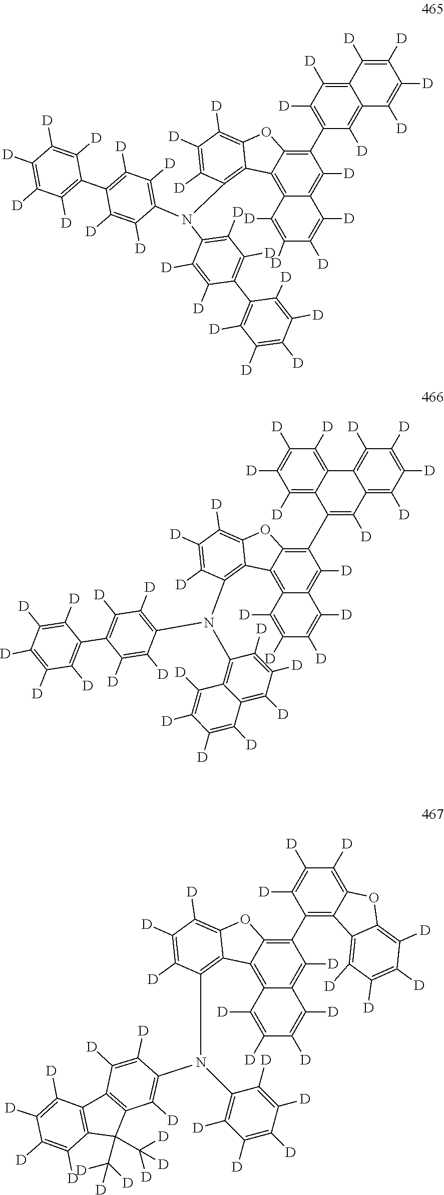

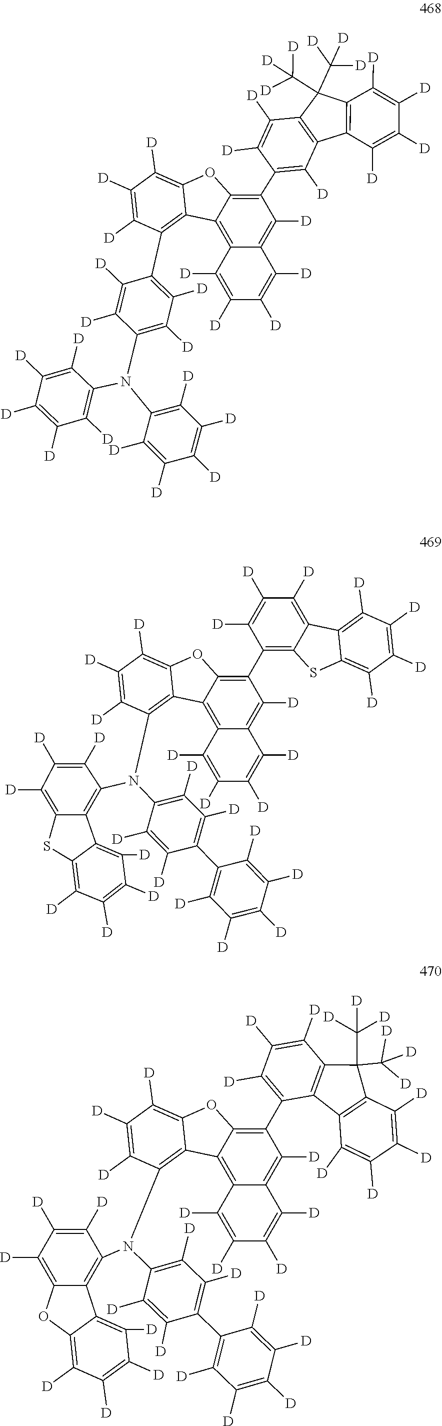

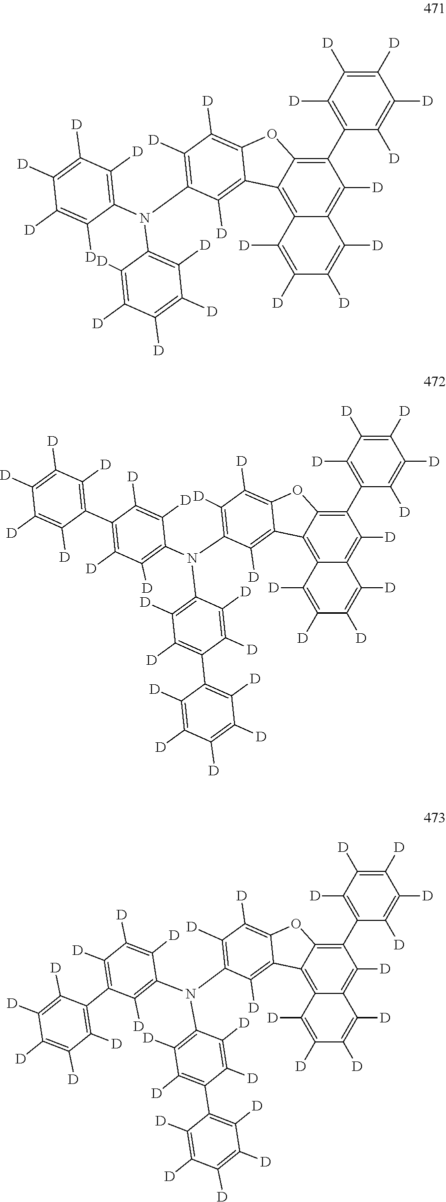

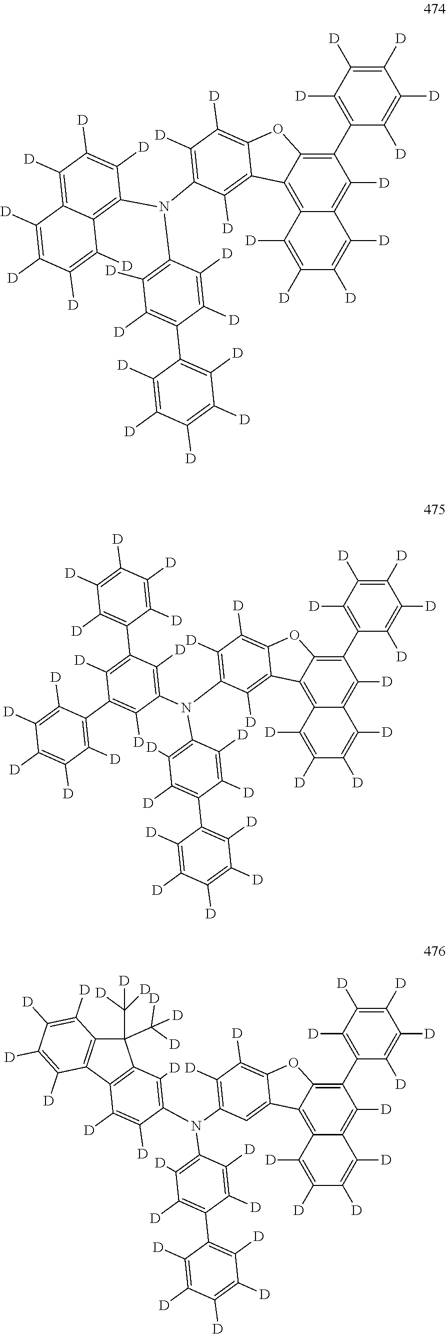

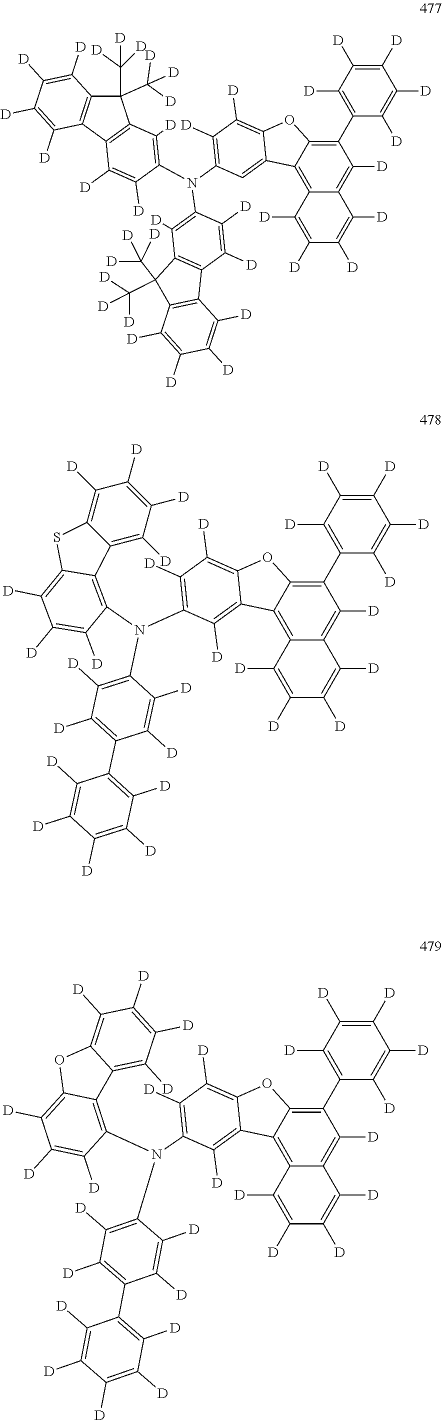

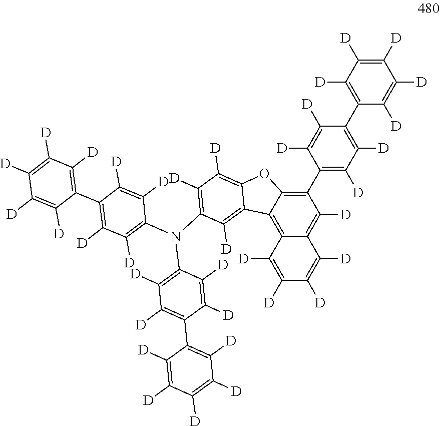 [SMILES: [2H]c1c([2H])c([2H])c(-c2c([2H])c([2H])c(-c3c([2H])c4c([2H])c([2H])c([2H])c([2H])c4c4c3oc3c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c34)c([2H])c2[2H])c([2H])c1[2H]]

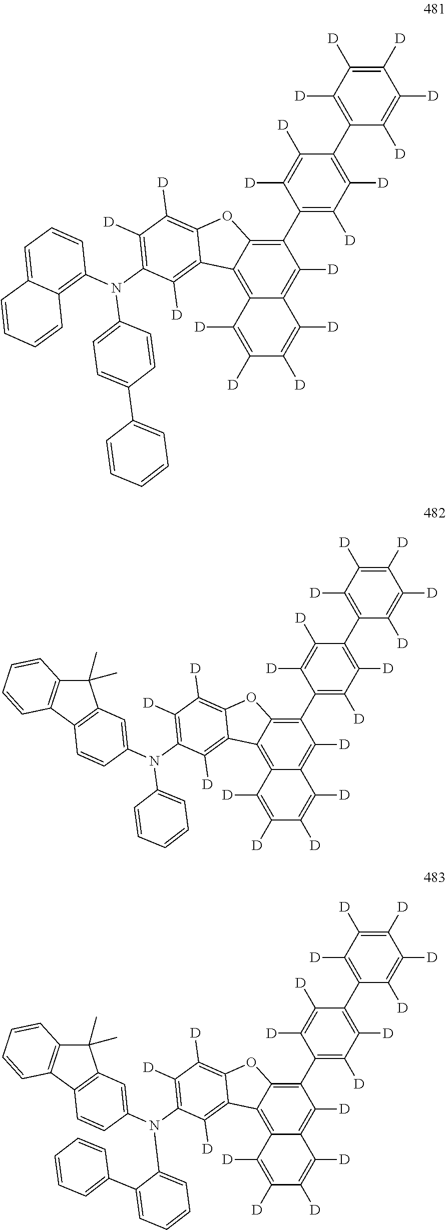

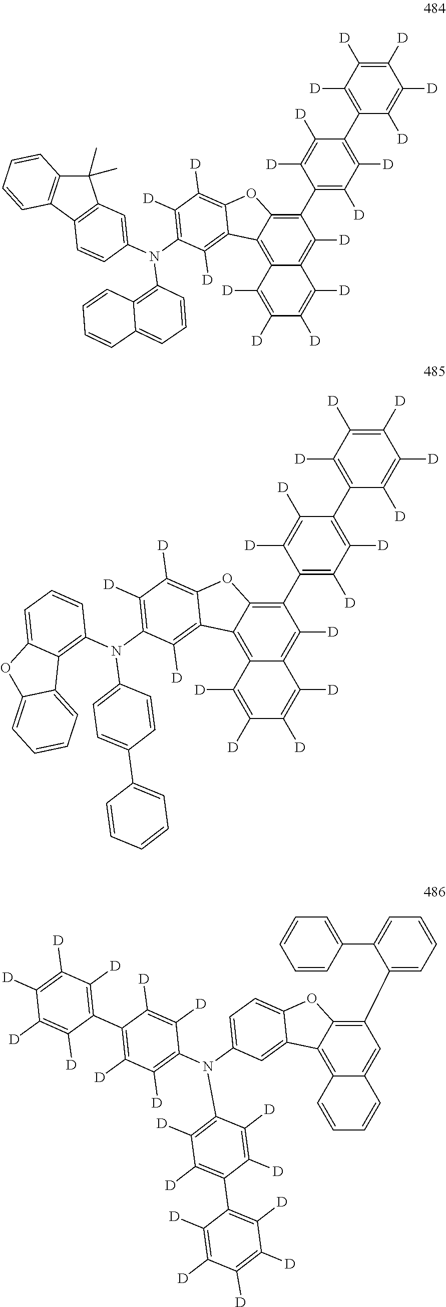

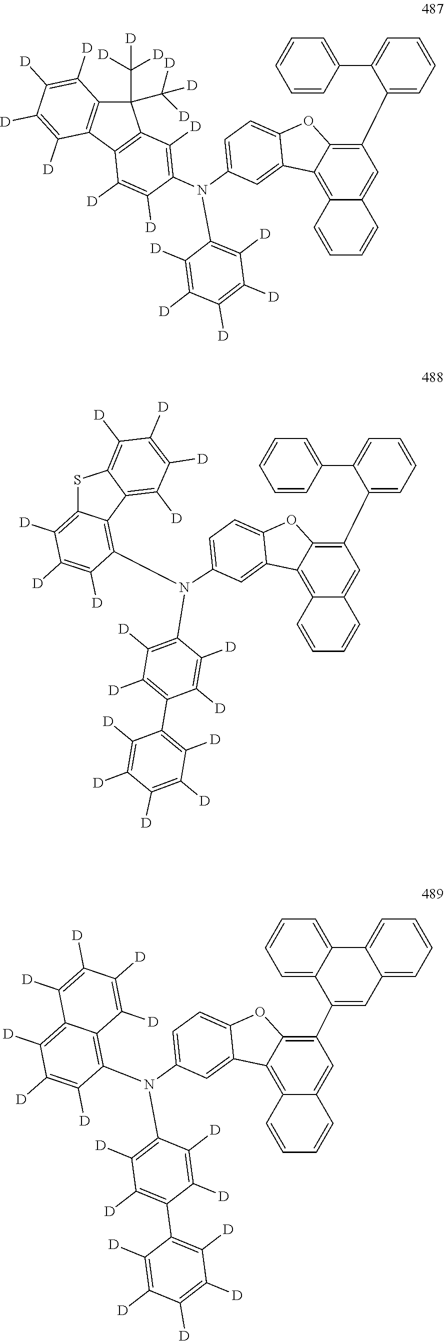

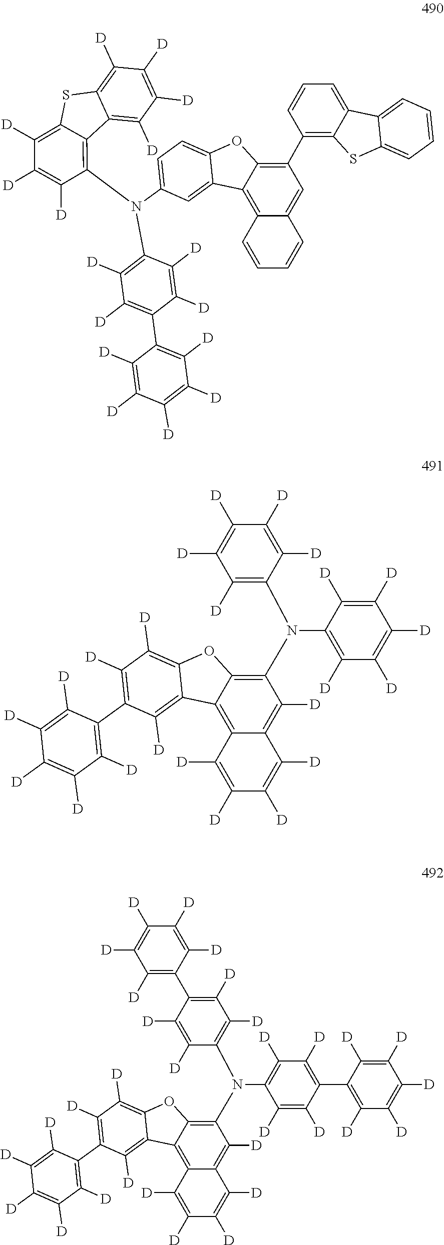

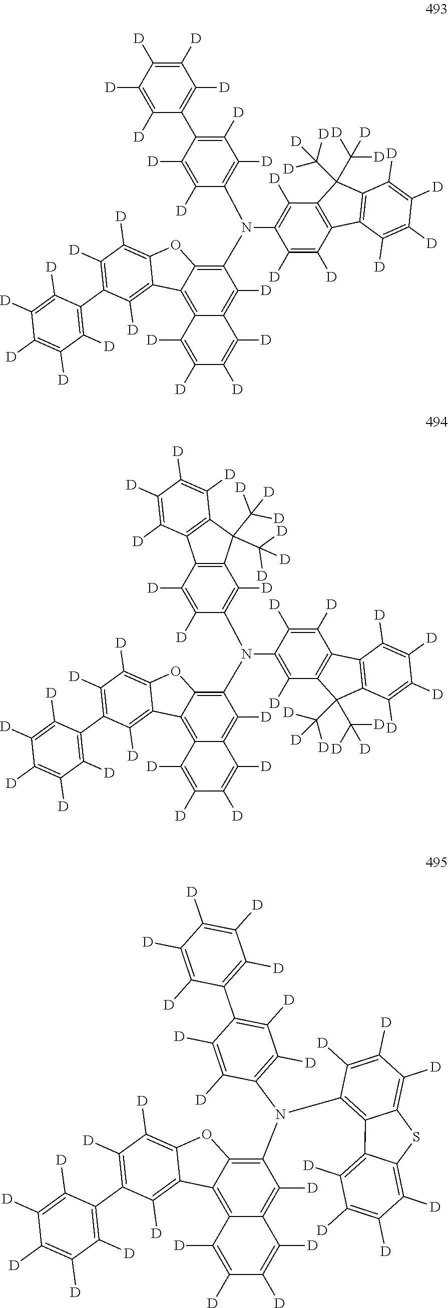

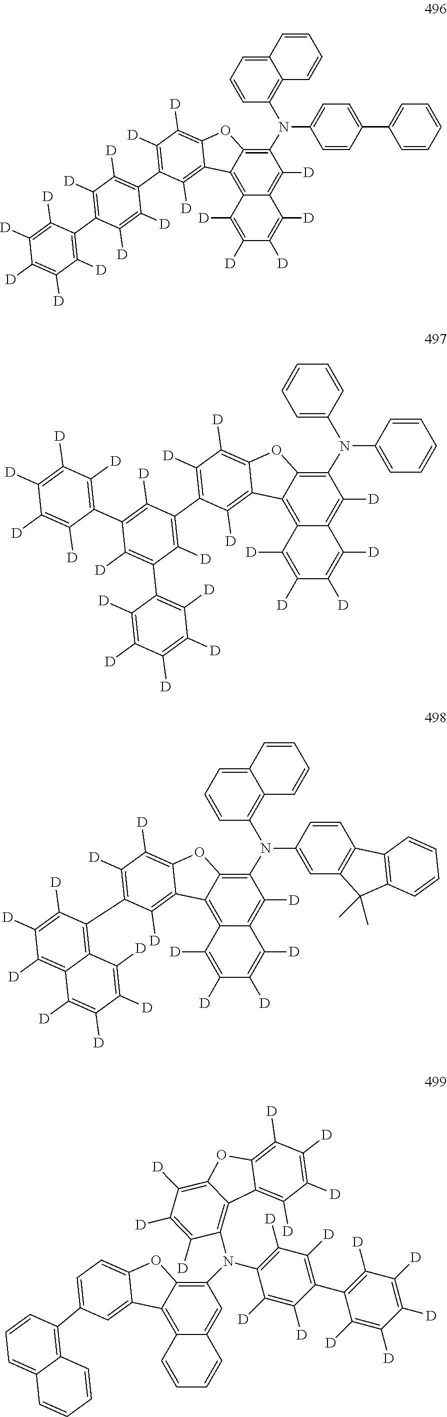

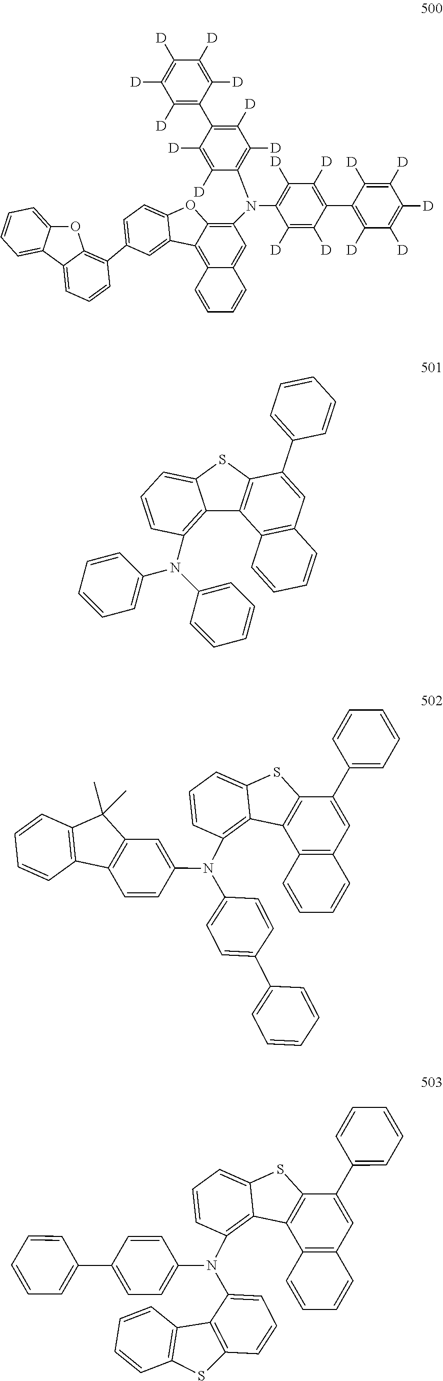 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4ccccc4c4c3oc3ccc(-c5cccc6c5oc5ccccc56)cc34)c([2H])c2[2H])c([2H])c1[2H] ; c1ccc(-c2cc3ccccc3c3c2sc2cccc(N(c4ccccc4)c4ccccc4)c23)cc1 ; CC1(C)c2ccccc2-c2ccc(N(c3ccc(-c4ccccc4)cc3)c3cccc4sc5c(-c6ccccc6)cc6ccccc6c5c34)cc21 ; c1ccc(-c2ccc(N(c3cccc4sc5ccccc5c34)c3cccc4sc5c(-c6ccccc6)cc6ccccc6c5c34)cc2)cc1]

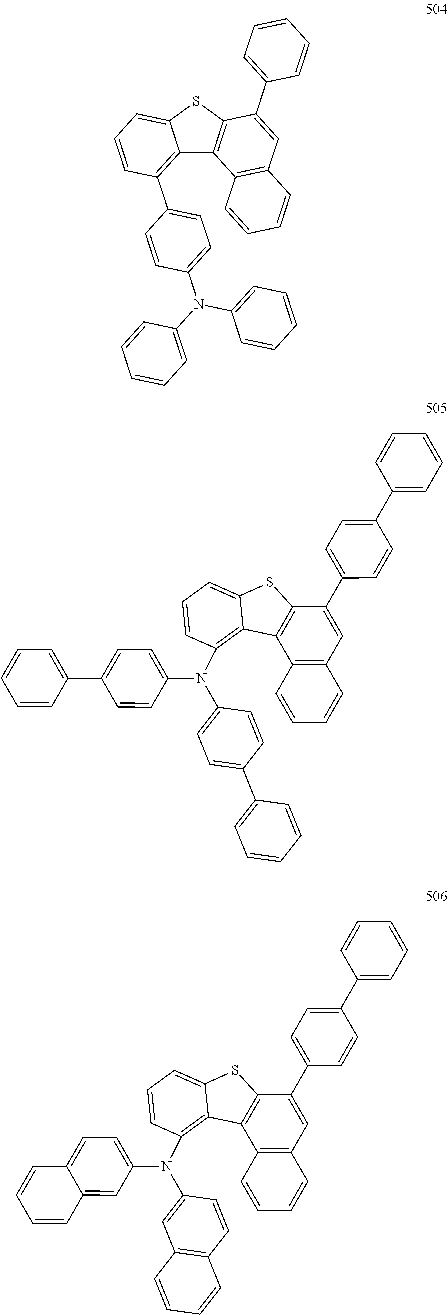 [SMILES: c1ccc(-c2cc3ccccc3c3c2sc2cccc(-c4ccc(N(c5ccccc5)c5ccccc5)cc4)c23)cc1 ; c1ccc(-c2ccc(-c3cc4ccccc4c4c3sc3cccc(N(c5ccc(-c6ccccc6)cc5)c5ccc(-c6ccccc6)cc5)c34)cc2)cc1 ; c1ccc(-c2ccc(-c3cc4ccccc4c4c3sc3cccc(N(c5ccc6ccccc6c5)c5ccc6ccccc6c5)c34)cc2)cc1]

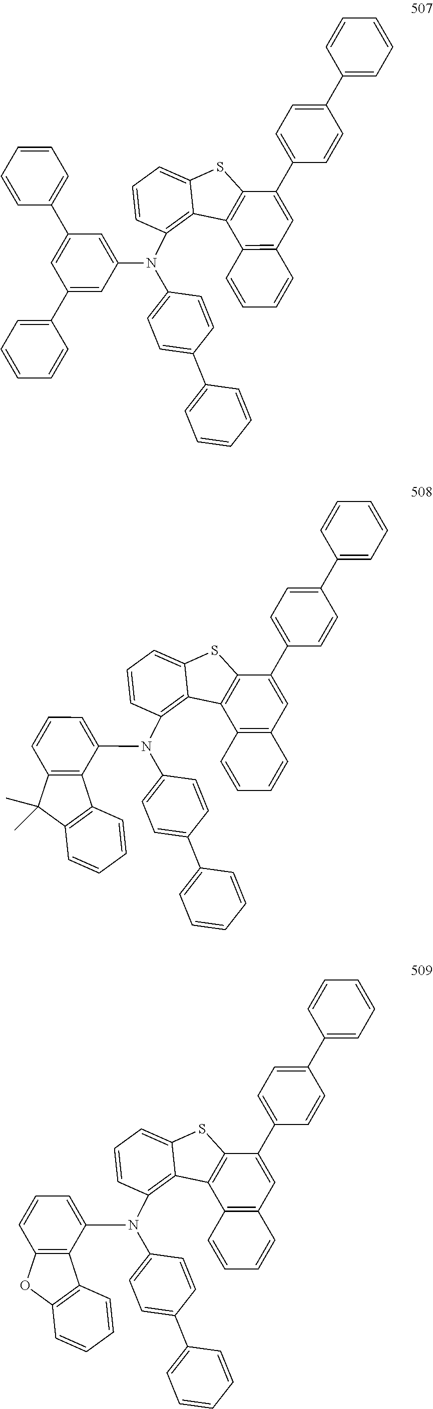 [SMILES: c1ccc(-c2ccc(-c3cc4ccccc4c4c3sc3cccc(N(c5ccc(-c6ccccc6)cc5)c5cc(-c6ccccc6)cc(-c6ccccc6)c5)c34)cc2)cc1 ; CC1(C)c2ccccc2-c2c(N(c3ccc(-c4ccccc4)cc3)c3cccc4sc5c(-c6ccc(-c7ccccc7)cc6)cc6ccccc6c5c34)cccc21 ; c1ccc(-c2ccc(-c3cc4ccccc4c4c3sc3cccc(N(c5ccc(-c6ccccc6)cc5)c5cccc6oc7ccccc7c56)c34)cc2)cc1]

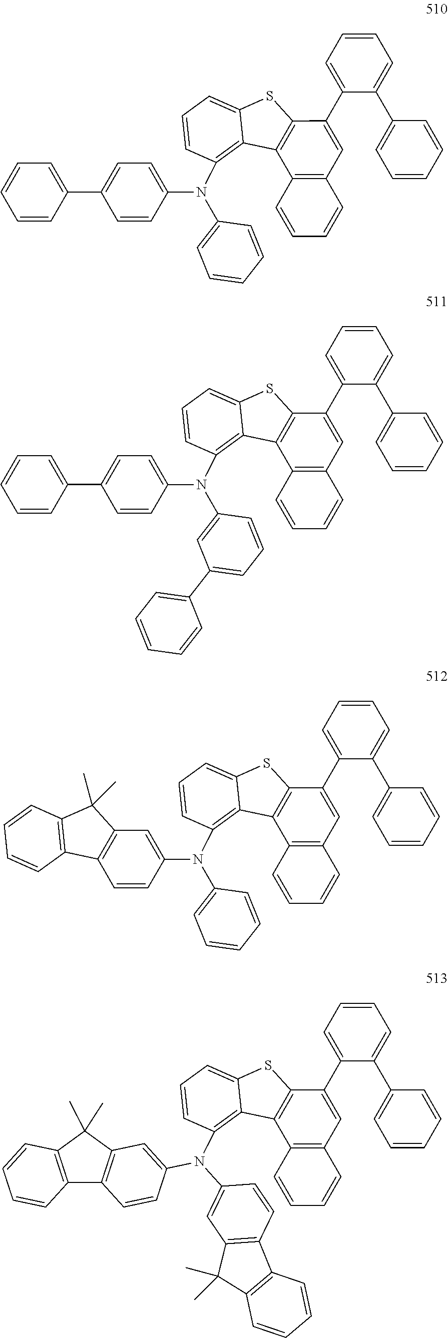 [SMILES: c1ccc(-c2ccc(N(c3ccccc3)c3cccc4sc5c(-c6ccccc6-c6ccccc6)cc6ccccc6c5c34)cc2)cc1 ; c1ccc(-c2ccc(N(c3cccc(-c4ccccc4)c3)c3cccc4sc5c(-c6ccccc6-c6ccccc6)cc6ccccc6c5c34)cc2)cc1 ; CC1(C)c2ccccc2-c2ccc(N(c3ccccc3)c3cccc4sc5c(-c6ccccc6-c6ccccc6)cc6ccccc6c5c34)cc21 ; CC1(C)c2ccccc2-c2ccc(N(c3ccc4c(c3)C(C)(C)c3ccccc3-4)c3cccc4sc5c(-c6ccccc6-c6ccccc6)cc6ccccc6c5c34)cc21]

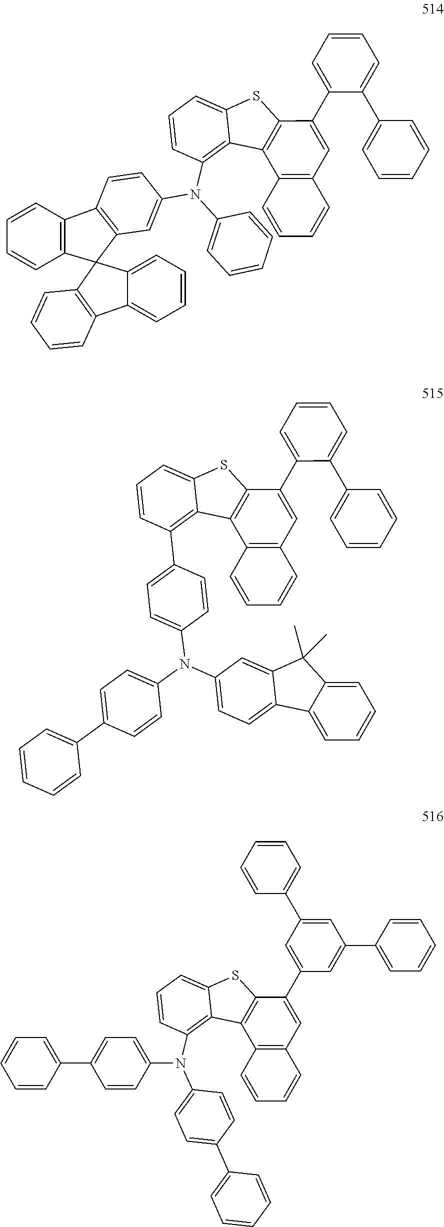 [SMILES: c1ccc(-c2ccccc2-c2cc3ccccc3c3c2sc2cccc(N(c4ccccc4)c4ccc5c(c4)C4(c6ccccc6-c6ccccc64)c4ccccc4-5)c23)cc1 ; CC1(C)c2ccccc2-c2ccc(N(c3ccc(-c4ccccc4)cc3)c3ccc(-c4cccc5sc6c(-c7ccccc7-c7ccccc7)cc7ccccc7c6c45)cc3)cc21 ; c1ccc(-c2ccc(N(c3ccc(-c4ccccc4)cc3)c3cccc4sc5c(-c6cc(-c7ccccc7)cc(-c7ccccc7)c6)cc6ccccc6c5c34)cc2)cc1]

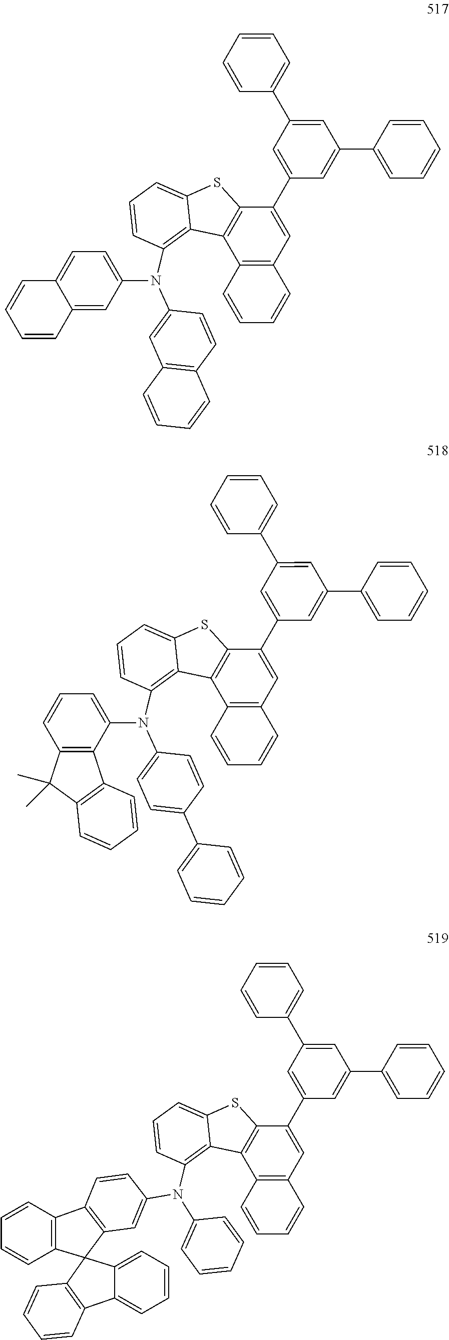 [SMILES: c1ccc(-c2cc(-c3ccccc3)cc(-c3cc4ccccc4c4c3sc3cccc(N(c5ccc6ccccc6c5)c5ccc6ccccc6c5)c34)c2)cc1 ; CC1(C)c2ccccc2-c2c(N(c3ccc(-c4ccccc4)cc3)c3cccc4sc5c(-c6cc(-c7ccccc7)cc(-c7ccccc7)c6)cc6ccccc6c5c34)cccc21 ; c1ccc(-c2cc(-c3ccccc3)cc(-c3cc4ccccc4c4c3sc3cccc(N(c5ccccc5)c5ccc6c(c5)C5(c7ccccc7-c7ccccc75)c5ccccc5-6)c34)c2)cc1]

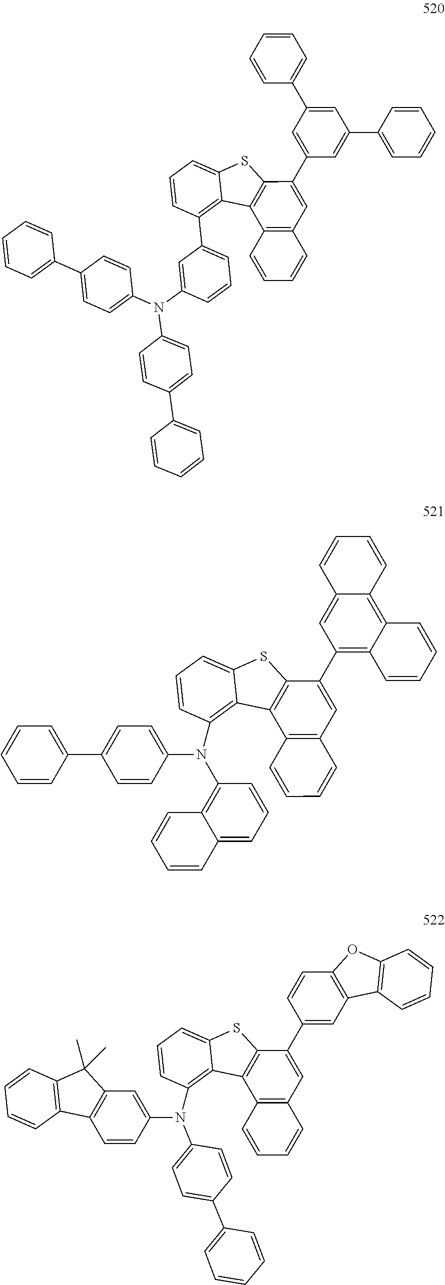 [SMILES: c1ccc(-c2ccc(N(c3ccc(-c4ccccc4)cc3)c3cccc(-c4cccc5sc6c(-c7cc(-c8ccccc8)cc(-c8ccccc8)c7)cc7ccccc7c6c45)c3)cc2)cc1 ; c1ccc(-c2ccc(N(c3cccc4ccccc34)c3cccc4sc5c(-c6cc7ccccc7c7ccccc67)cc6ccccc6c5c34)cc2)cc1 ; CC1(C)c2ccccc2-c2ccc(N(c3ccc(-c4ccccc4)cc3)c3cccc4sc5c(-c6ccc7oc8ccccc8c7c6)cc6ccccc6c5c34)cc21]

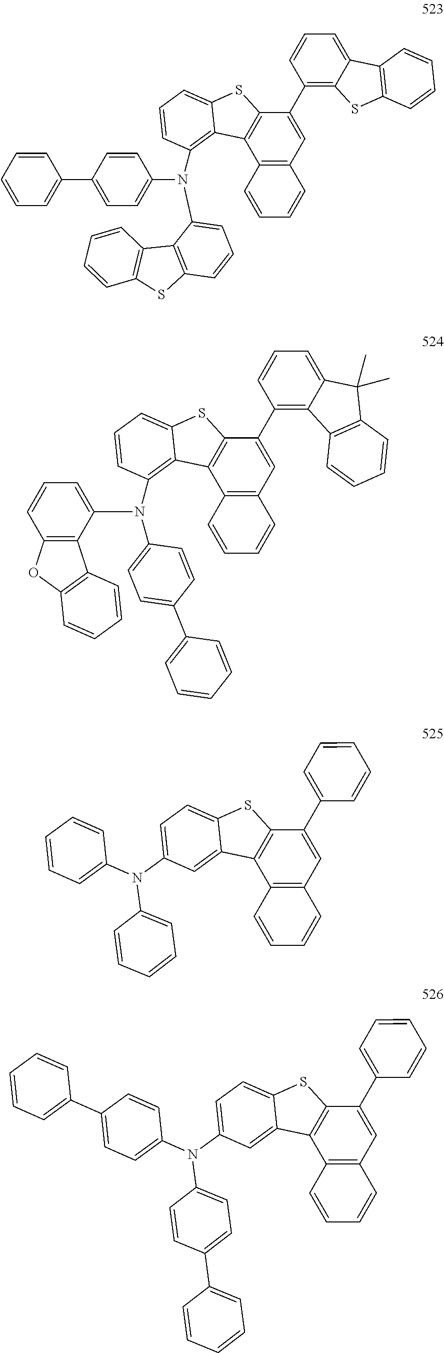 [SMILES: c1ccc(-c2ccc(N(c3cccc4sc5ccccc5c34)c3cccc4sc5c(-c6cccc7c6sc6ccccc67)cc6ccccc6c5c34)cc2)cc1 ; CC1(C)c2ccccc2-c2c(-c3cc4ccccc4c4c3sc3cccc(N(c5ccc(-c6ccccc6)cc5)c5cccc6oc7ccccc7c56)c34)cccc21 ; c1ccc(-c2cc3ccccc3c3c2sc2ccc(N(c4ccccc4)c4ccccc4)cc23)cc1 ; c1ccc(-c2ccc(N(c3ccc(-c4ccccc4)cc3)c3ccc4sc5c(-c6ccccc6)cc6ccccc6c5c4c3)cc2)cc1]

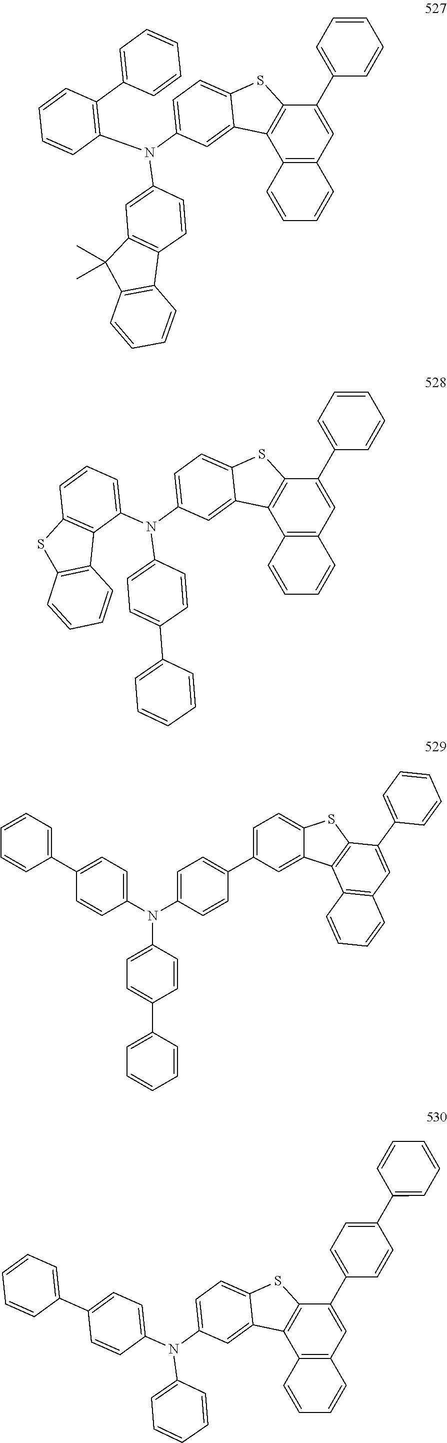 [SMILES: CC1(C)c2ccccc2-c2ccc(N(c3ccc4sc5c(-c6ccccc6)cc6ccccc6c5c4c3)c3ccccc3-c3ccccc3)cc21 ; c1ccc(-c2ccc(N(c3ccc4sc5c(-c6ccccc6)cc6ccccc6c5c4c3)c3cccc4sc5ccccc5c34)cc2)cc1 ; c1ccc(-c2ccc(N(c3ccc(-c4ccccc4)cc3)c3ccc(-c4ccc5sc6c(-c7ccccc7)cc7ccccc7c6c5c4)cc3)cc2)cc1 ; c1ccc(-c2ccc(-c3cc4ccccc4c4c3sc3ccc(N(c5ccccc5)c5ccc(-c6ccccc6)cc5)cc34)cc2)cc1]

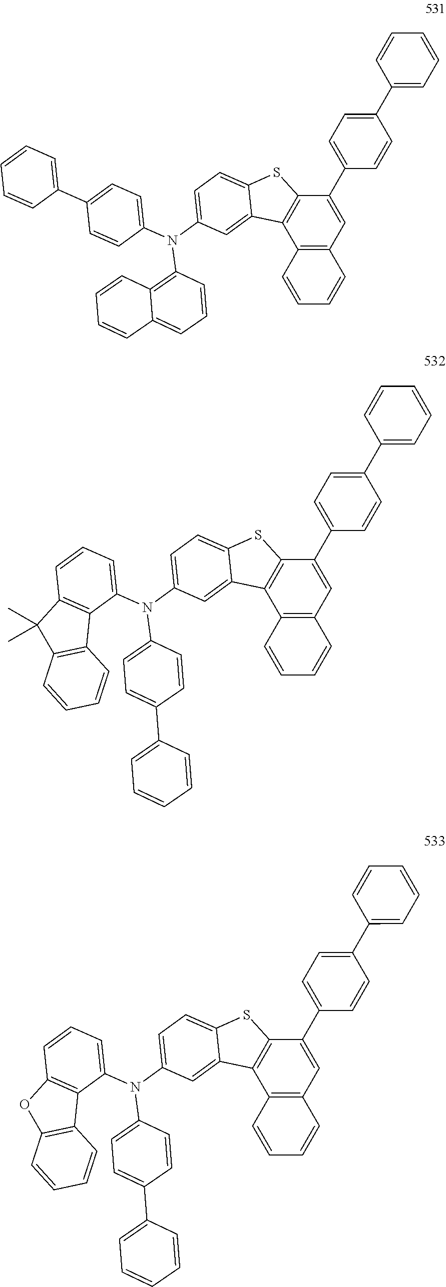 [SMILES: c1ccc(-c2ccc(-c3cc4ccccc4c4c3sc3ccc(N(c5ccc(-c6ccccc6)cc5)c5cccc6ccccc56)cc34)cc2)cc1 ; CC1(C)c2ccccc2-c2c(N(c3ccc(-c4ccccc4)cc3)c3ccc4sc5c(-c6ccc(-c7ccccc7)cc6)cc6ccccc6c5c4c3)cccc21 ; c1ccc(-c2ccc(-c3cc4ccccc4c4c3sc3ccc(N(c5ccc(-c6ccccc6)cc5)c5cccc6oc7ccccc7c56)cc34)cc2)cc1]

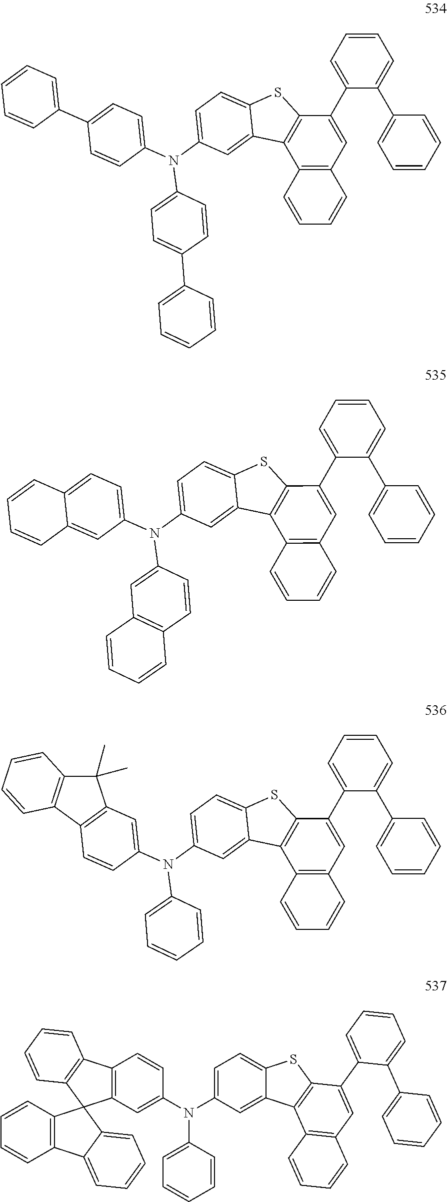 [SMILES: c1ccc(-c2ccc(N(c3ccc(-c4ccccc4)cc3)c3ccc4sc5c(-c6ccccc6-c6ccccc6)cc6ccccc6c5c4c3)cc2)cc1 ; c1ccc(-c2ccccc2-c2cc3ccccc3c3c2sc2ccc(N(c4ccc5ccccc5c4)c4ccc5ccccc5c4)cc23)cc1 ; CC1(C)c2ccccc2-c2ccc(N(c3ccccc3)c3ccc4sc5c(-c6ccccc6-c6ccccc6)cc6ccccc6c5c4c3)cc21 ; c1ccc(-c2ccccc2-c2cc3ccccc3c3c2sc2ccc(N(c4ccccc4)c4ccc5c(c4)C4(c6ccccc6-c6ccccc64)c4ccccc4-5)cc23)cc1]

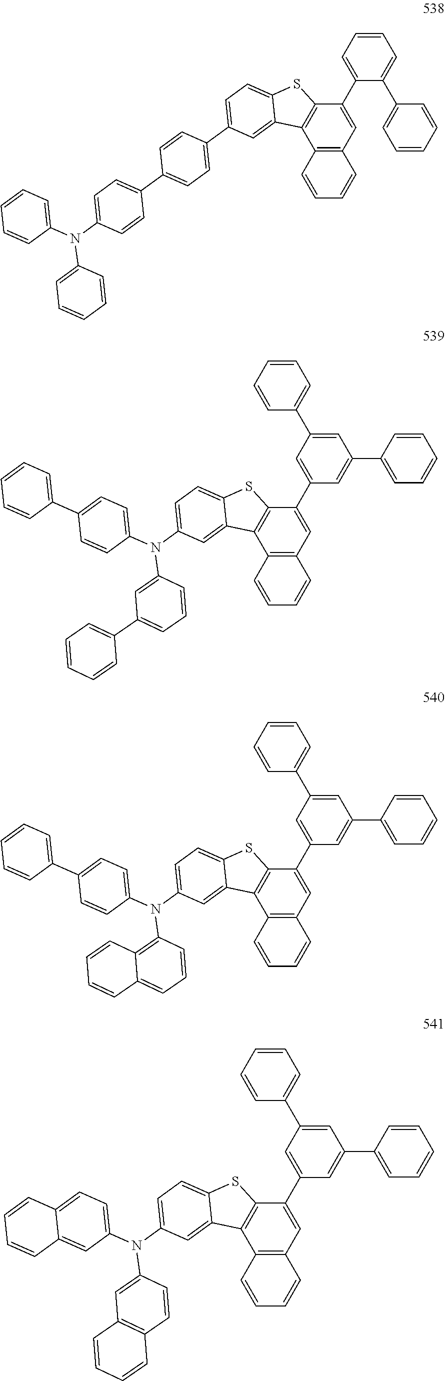 [SMILES: c1ccc(-c2ccccc2-c2cc3ccccc3c3c2sc2ccc(-c4ccc(-c5ccc(N(c6ccccc6)c6ccccc6)cc5)cc4)cc23)cc1 ; c1ccc(-c2ccc(N(c3cccc(-c4ccccc4)c3)c3ccc4sc5c(-c6cc(-c7ccccc7)cc(-c7ccccc7)c6)cc6ccccc6c5c4c3)cc2)cc1 ; c1ccc(-c2ccc(N(c3ccc4sc5c(-c6cc(-c7ccccc7)cc(-c7ccccc7)c6)cc6ccccc6c5c4c3)c3cccc4ccccc34)cc2)cc1 ; c1ccc(-c2cc(-c3ccccc3)cc(-c3cc4ccccc4c4c3sc3ccc(N(c5ccc6ccccc6c5)c5ccc6ccccc6c5)cc34)c2)cc1]

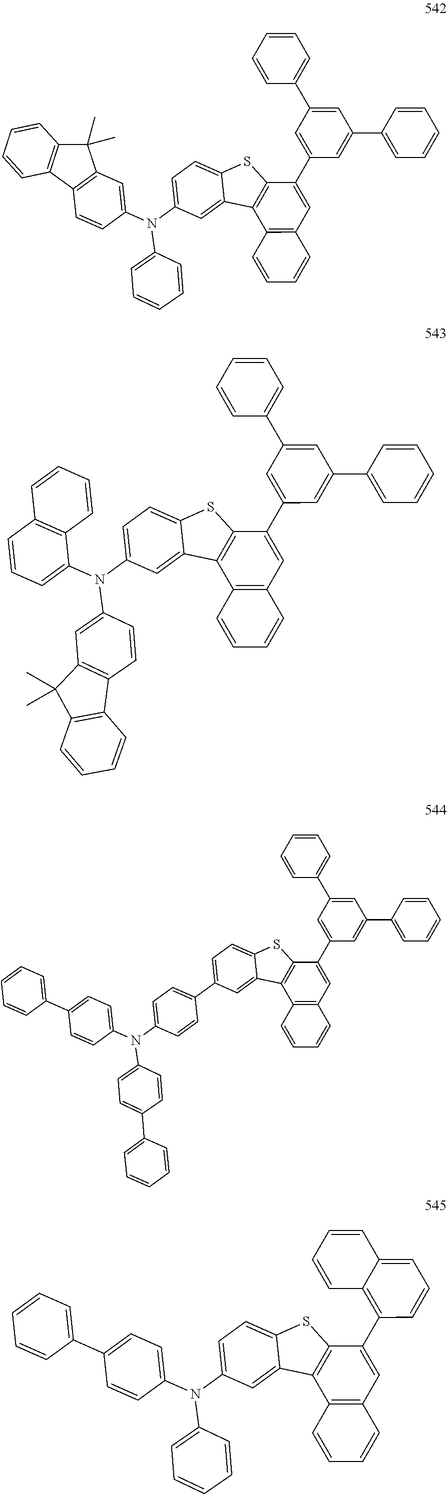 [SMILES: CC1(C)c2ccccc2-c2ccc(N(c3ccccc3)c3ccc4sc5c(-c6cc(-c7ccccc7)cc(-c7ccccc7)c6)cc6ccccc6c5c4c3)cc21 ; CC1(C)c2ccccc2-c2ccc(N(c3ccc4sc5c(-c6cc(-c7ccccc7)cc(-c7ccccc7)c6)cc6ccccc6c5c4c3)c3cccc4ccccc34)cc21 ; c1ccc(-c2ccc(N(c3ccc(-c4ccccc4)cc3)c3ccc(-c4ccc5sc6c(-c7cc(-c8ccccc8)cc(-c8ccccc8)c7)cc7ccccc7c6c5c4)cc3)cc2)cc1 ; c1ccc(-c2ccc(N(c3ccccc3)c3ccc4sc5c(-c6cccc7ccccc67)cc6ccccc6c5c4c3)cc2)cc1]

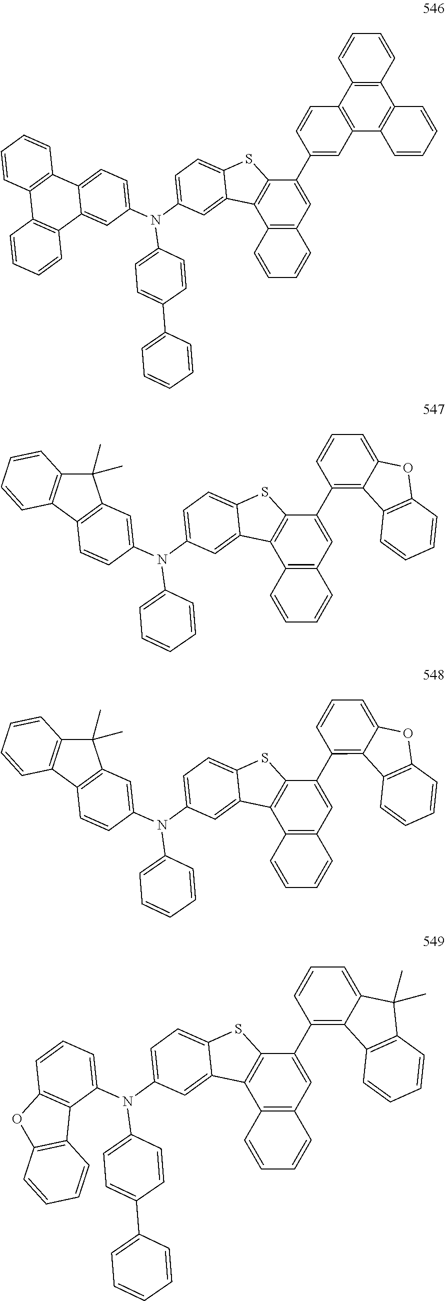 [SMILES: c1ccc(-c2ccc(N(c3ccc4c5ccccc5c5ccccc5c4c3)c3ccc4sc5c(-c6ccc7c8ccccc8c8ccccc8c7c6)cc6ccccc6c5c4c3)cc2)cc1 ; CC1(C)c2ccccc2-c2ccc(N(c3ccccc3)c3ccc4sc5c(-c6cccc7oc8ccccc8c67)cc6ccccc6c5c4c3)cc21 ; CC1(C)c2ccccc2-c2ccc(N(c3ccccc3)c3ccc4sc5c(-c6cccc7oc8ccccc8c67)cc6ccccc6c5c4c3)cc21 ; CC1(C)c2ccccc2-c2c(-c3cc4ccccc4c4c3sc3ccc(N(c5ccc(-c6ccccc6)cc5)c5cccc6oc7ccccc7c56)cc34)cccc21]

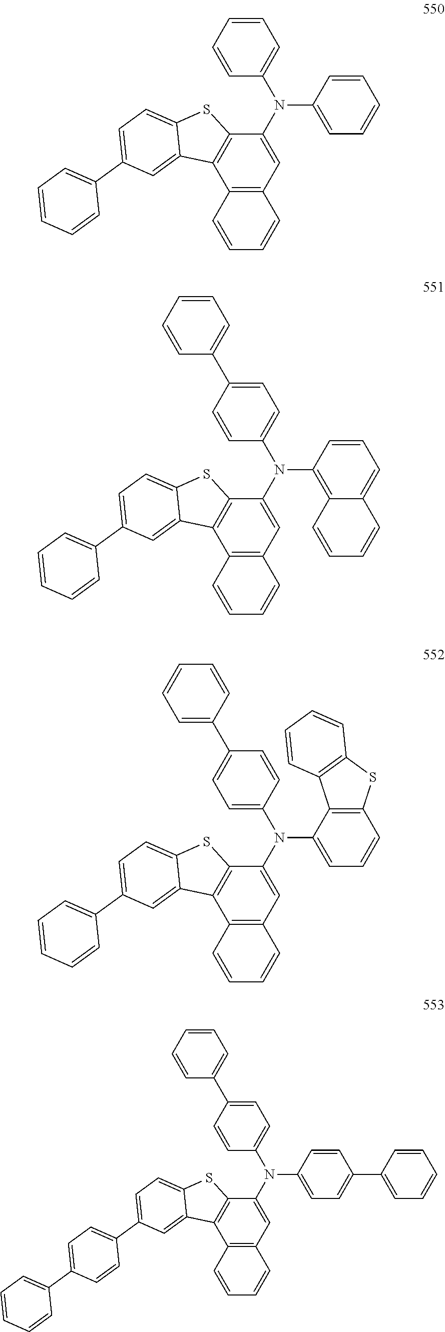 [SMILES: c1ccc(-c2ccc3sc4c(N(c5ccccc5)c5ccccc5)cc5ccccc5c4c3c2)cc1 ; c1ccc(-c2ccc(N(c3cccc4ccccc34)c3cc4ccccc4c4c3sc3ccc(-c5ccccc5)cc34)cc2)cc1 ; c1ccc(-c2ccc(N(c3cc4ccccc4c4c3sc3ccc(-c5ccccc5)cc34)c3cccc4sc5ccccc5c34)cc2)cc1 ; c1ccc(-c2ccc(-c3ccc4sc5c(N(c6ccc(-c7ccccc7)cc6)c6ccc(-c7ccccc7)cc6)cc6ccccc6c5c4c3)cc2)cc1]

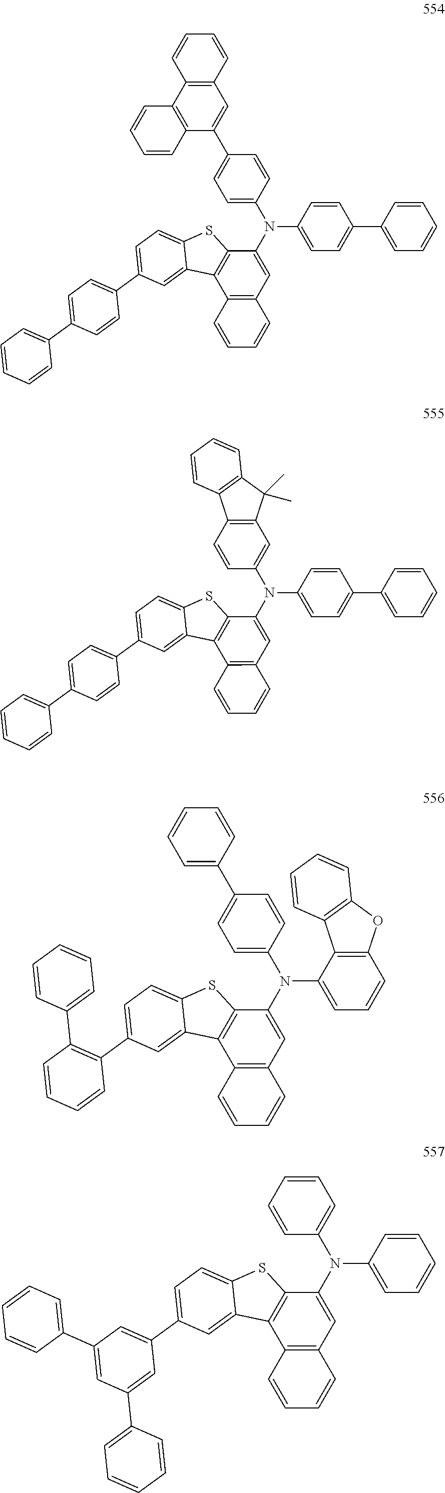 [SMILES: c1ccc(-c2ccc(-c3ccc4sc5c(N(c6ccc(-c7ccccc7)cc6)c6ccc(-c7cc8ccccc8c8ccccc78)cc6)cc6ccccc6c5c4c3)cc2)cc1 ; CC1(C)c2ccccc2-c2ccc(N(c3ccc(-c4ccccc4)cc3)c3cc4ccccc4c4c3sc3ccc(-c5ccc(-c6ccccc6)cc5)cc34)cc21 ; c1ccc(-c2ccc(N(c3cc4ccccc4c4c3sc3ccc(-c5ccccc5-c5ccccc5)cc34)c3cccc4oc5ccccc5c34)cc2)cc1 ; c1ccc(-c2cc(-c3ccccc3)cc(-c3ccc4sc5c(N(c6ccccc6)c6ccccc6)cc6ccccc6c5c4c3)c2)cc1]

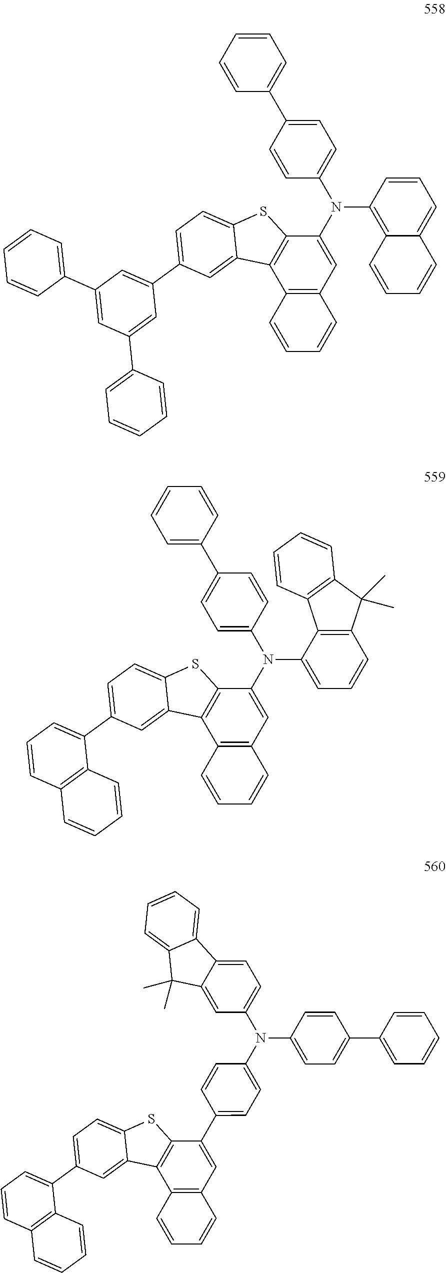 [SMILES: c1ccc(-c2ccc(N(c3cccc4ccccc34)c3cc4ccccc4c4c3sc3ccc(-c5cc(-c6ccccc6)cc(-c6ccccc6)c5)cc34)cc2)cc1 ; CC1(C)c2ccccc2-c2c(N(c3ccc(-c4ccccc4)cc3)c3cc4ccccc4c4c3sc3ccc(-c5cccc6ccccc56)cc34)cccc21 ; CC1(C)c2ccccc2-c2ccc(N(c3ccc(-c4ccccc4)cc3)c3ccc(-c4cc5ccccc5c5c4sc4ccc(-c6cccc7ccccc67)cc45)cc3)cc21]

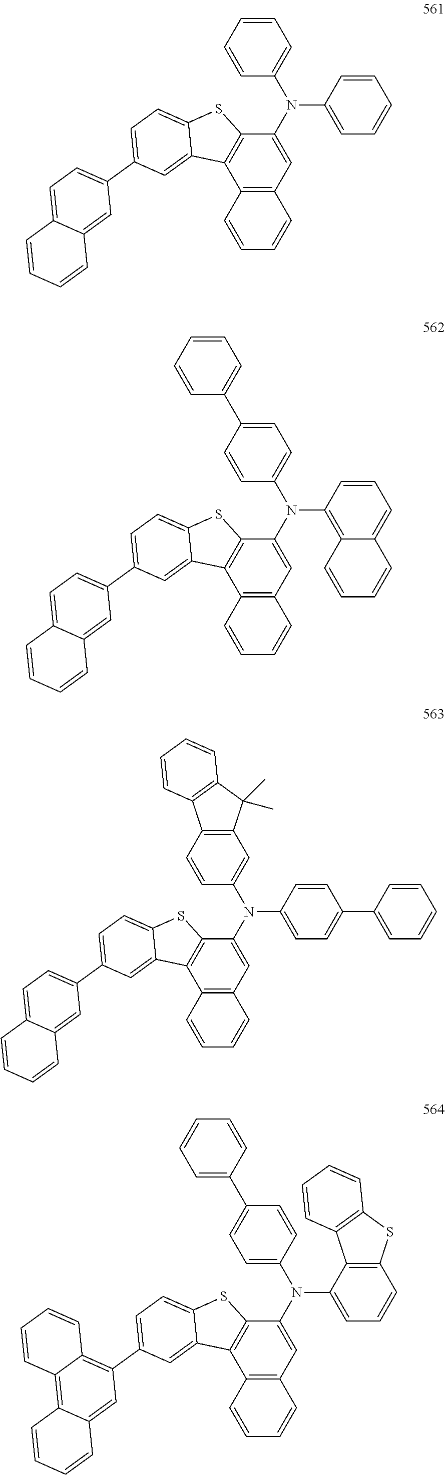 [SMILES: c1ccc(N(c2ccccc2)c2cc3ccccc3c3c2sc2ccc(-c4ccc5ccccc5c4)cc23)cc1 ; c1ccc(-c2ccc(N(c3cccc4ccccc34)c3cc4ccccc4c4c3sc3ccc(-c5ccc6ccccc6c5)cc34)cc2)cc1 ; CC1(C)c2ccccc2-c2ccc(N(c3ccc(-c4ccccc4)cc3)c3cc4ccccc4c4c3sc3ccc(-c5ccc6ccccc6c5)cc34)cc21 ; c1ccc(-c2ccc(N(c3cc4ccccc4c4c3sc3ccc(-c5cc6ccccc6c6ccccc56)cc34)c3cccc4sc5ccccc5c34)cc2)cc1]

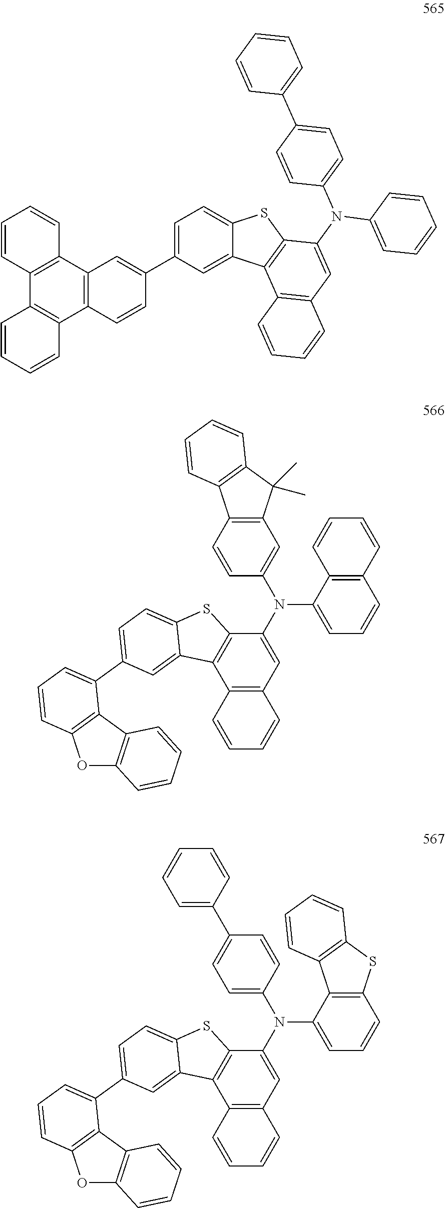 [SMILES: c1ccc(-c2ccc(N(c3ccccc3)c3cc4ccccc4c4c3sc3ccc(-c5ccc6c7ccccc7c7ccccc7c6c5)cc34)cc2)cc1 ; CC1(C)c2ccccc2-c2ccc(N(c3cccc4ccccc34)c3cc4ccccc4c4c3sc3ccc(-c5cccc6oc7ccccc7c56)cc34)cc21 ; c1ccc(-c2ccc(N(c3cc4ccccc4c4c3sc3ccc(-c5cccc6oc7ccccc7c56)cc34)c3cccc4sc5ccccc5c34)cc2)cc1]

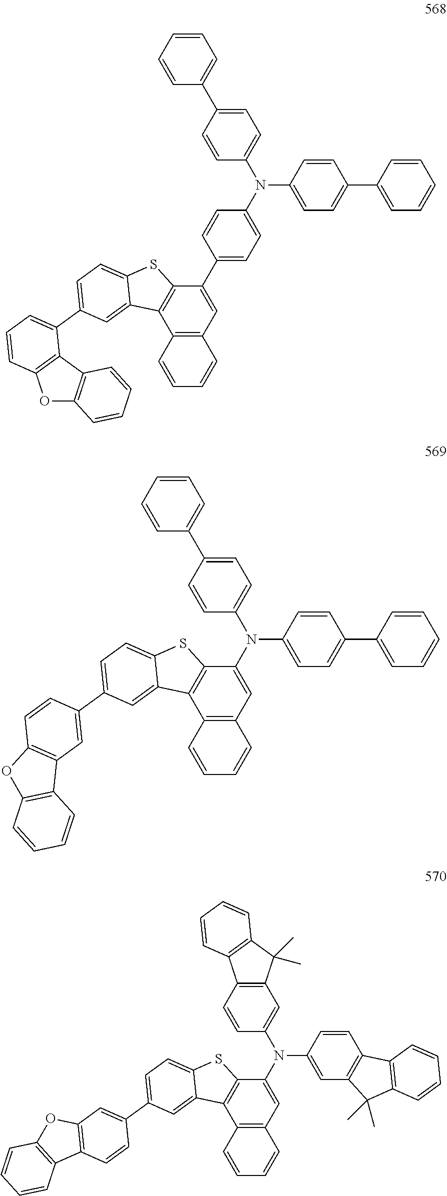 [SMILES: c1ccc(-c2ccc(N(c3ccc(-c4ccccc4)cc3)c3ccc(-c4cc5ccccc5c5c4sc4ccc(-c6cccc7oc8ccccc8c67)cc45)cc3)cc2)cc1 ; c1ccc(-c2ccc(N(c3ccc(-c4ccccc4)cc3)c3cc4ccccc4c4c3sc3ccc(-c5ccc6oc7ccccc7c6c5)cc34)cc2)cc1 ; CC1(C)c2ccccc2-c2ccc(N(c3ccc4c(c3)C(C)(C)c3ccccc3-4)c3cc4ccccc4c4c3sc3ccc(-c5ccc6c(c5)oc5ccccc56)cc34)cc21]

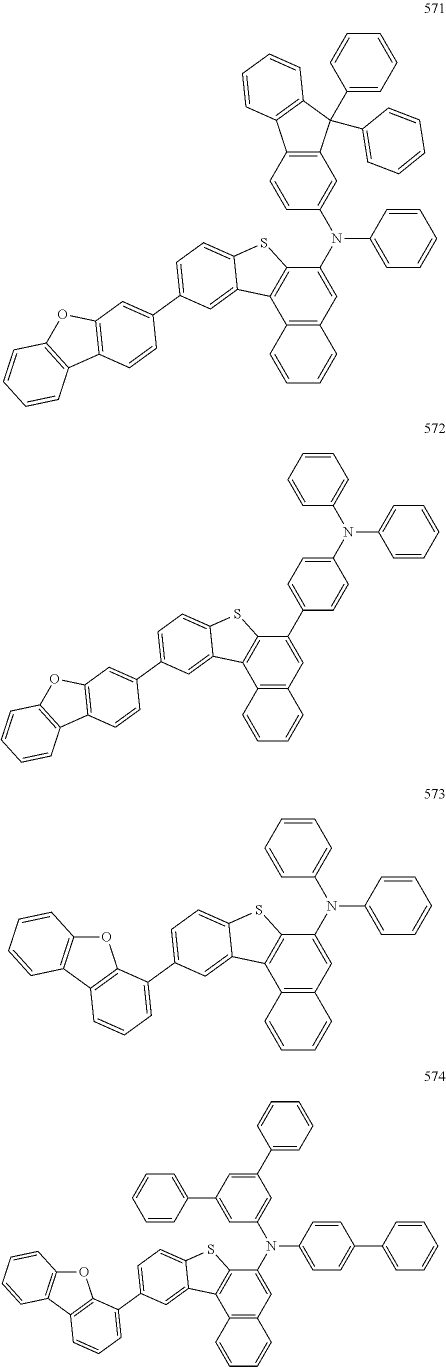 [SMILES: c1ccc(N(c2ccc3c(c2)C(c2ccccc2)(c2ccccc2)c2ccccc2-3)c2cc3ccccc3c3c2sc2ccc(-c4ccc5c(c4)oc4ccccc45)cc23)cc1 ; c1ccc(N(c2ccccc2)c2ccc(-c3cc4ccccc4c4c3sc3ccc(-c5ccc6c(c5)oc5ccccc56)cc34)cc2)cc1 ; c1ccc(N(c2ccccc2)c2cc3ccccc3c3c2sc2ccc(-c4cccc5c4oc4ccccc45)cc23)cc1 ; c1ccc(-c2ccc(N(c3cc(-c4ccccc4)cc(-c4ccccc4)c3)c3cc4ccccc4c4c3sc3ccc(-c5cccc6c5oc5ccccc56)cc34)cc2)cc1]

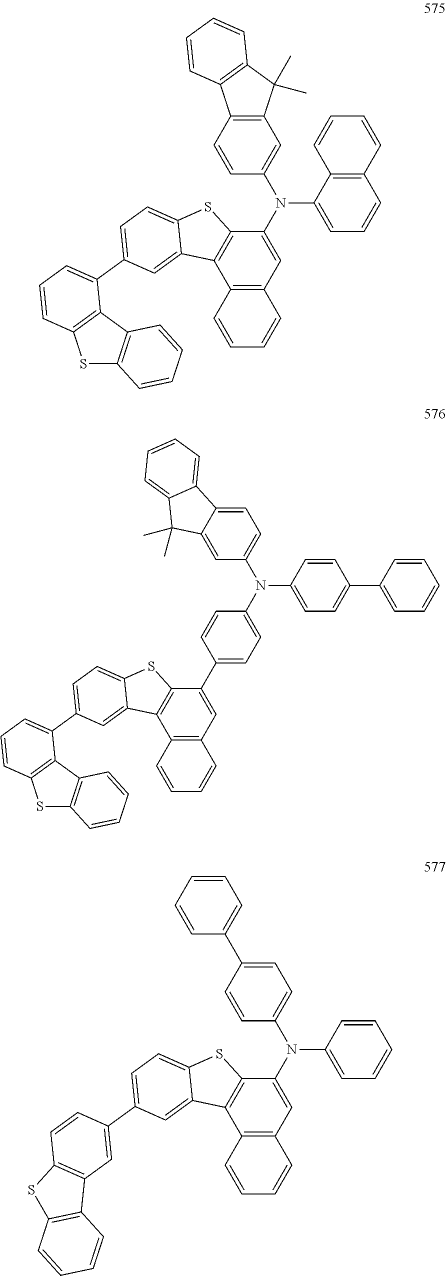 [SMILES: CC1(C)c2ccccc2-c2ccc(N(c3cccc4ccccc34)c3cc4ccccc4c4c3sc3ccc(-c5cccc6sc7ccccc7c56)cc34)cc21 ; CC1(C)c2ccccc2-c2ccc(N(c3ccc(-c4ccccc4)cc3)c3ccc(-c4cc5ccccc5c5c4sc4ccc(-c6cccc7sc8ccccc8c67)cc45)cc3)cc21 ; c1ccc(-c2ccc(N(c3ccccc3)c3cc4ccccc4c4c3sc3ccc(-c5ccc6sc7ccccc7c6c5)cc34)cc2)cc1]

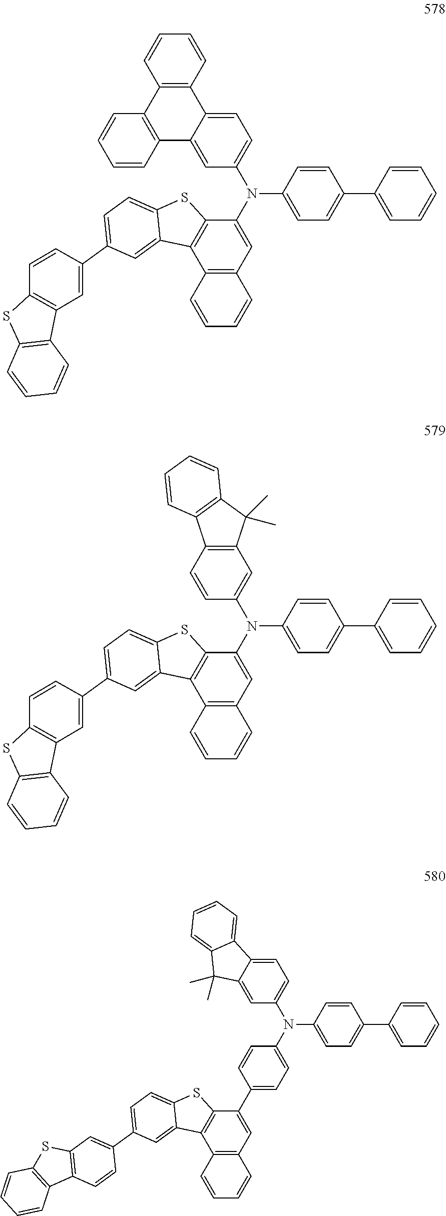 [SMILES: c1ccc(-c2ccc(N(c3ccc4c5ccccc5c5ccccc5c4c3)c3cc4ccccc4c4c3sc3ccc(-c5ccc6sc7ccccc7c6c5)cc34)cc2)cc1 ; CC1(C)c2ccccc2-c2ccc(N(c3ccc(-c4ccccc4)cc3)c3cc4ccccc4c4c3sc3ccc(-c5ccc6sc7ccccc7c6c5)cc34)cc21 ; CC1(C)c2ccccc2-c2ccc(N(c3ccc(-c4ccccc4)cc3)c3ccc(-c4cc5ccccc5c5c4sc4ccc(-c6ccc7c(c6)sc6ccccc67)cc45)cc3)cc21]

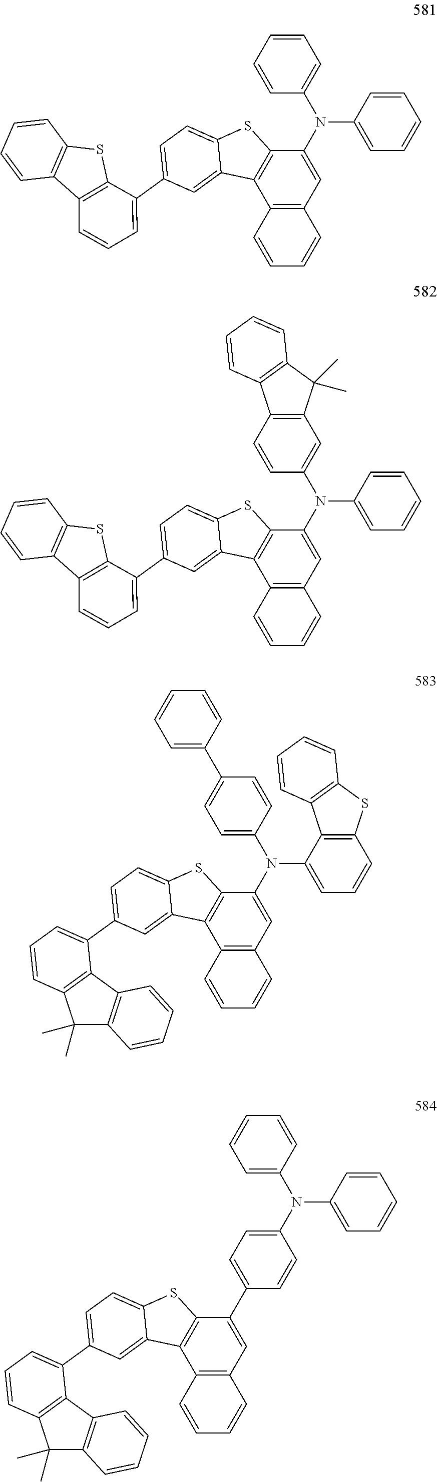 [SMILES: c1ccc(N(c2ccccc2)c2cc3ccccc3c3c2sc2ccc(-c4cccc5c4sc4ccccc45)cc23)cc1 ; CC1(C)c2ccccc2-c2ccc(N(c3ccccc3)c3cc4ccccc4c4c3sc3ccc(-c5cccc6c5sc5ccccc56)cc34)cc21 ; CC1(C)c2ccccc2-c2c(-c3ccc4sc5c(N(c6ccc(-c7ccccc7)cc6)c6cccc7sc8ccccc8c67)cc6ccccc6c5c4c3)cccc21 ; CC1(C)c2ccccc2-c2c(-c3ccc4sc5c(-c6ccc(N(c7ccccc7)c7ccccc7)cc6)cc6ccccc6c5c4c3)cccc21]

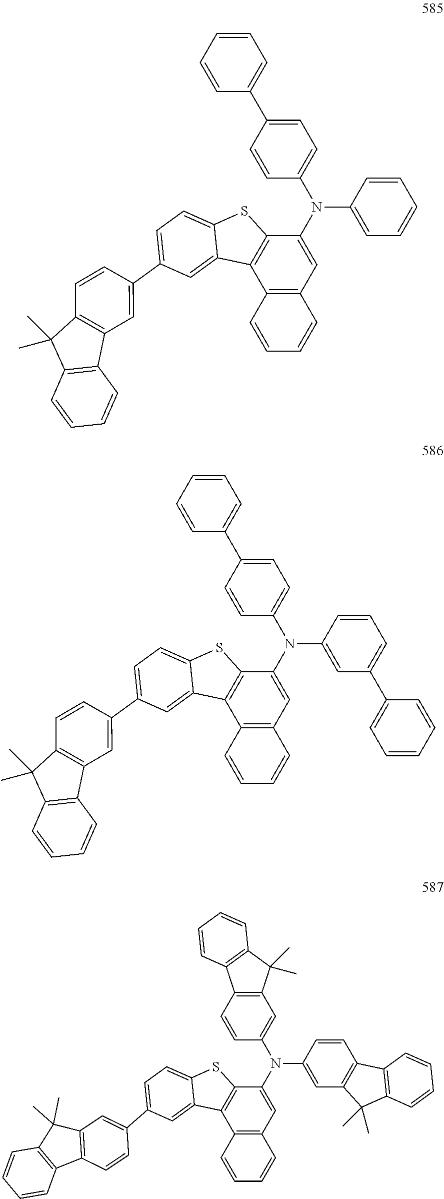 [SMILES: CC1(C)c2ccccc2-c2cc(-c3ccc4sc5c(N(c6ccccc6)c6ccc(-c7ccccc7)cc6)cc6ccccc6c5c4c3)ccc21 ; CC1(C)c2ccccc2-c2cc(-c3ccc4sc5c(N(c6ccc(-c7ccccc7)cc6)c6cccc(-c7ccccc7)c6)cc6ccccc6c5c4c3)ccc21 ; CC1(C)c2ccccc2-c2ccc(-c3ccc4sc5c(N(c6ccc7c(c6)C(C)(C)c6ccccc6-7)c6ccc7c(c6)C(C)(C)c6ccccc6-7)cc6ccccc6c5c4c3)cc21]

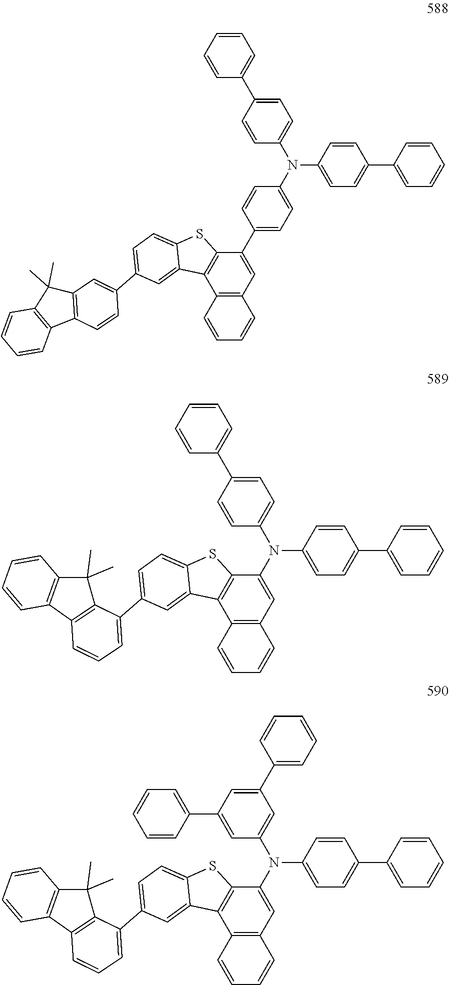 [SMILES: CC1(C)c2ccccc2-c2ccc(-c3ccc4sc5c(-c6ccc(N(c7ccc(-c8ccccc8)cc7)c7ccc(-c8ccccc8)cc7)cc6)cc6ccccc6c5c4c3)cc21 ; CC1(C)c2ccccc2-c2cccc(-c3ccc4sc5c(N(c6ccc(-c7ccccc7)cc6)c6ccc(-c7ccccc7)cc6)cc6ccccc6c5c4c3)c21 ; CC1(C)c2ccccc2-c2cccc(-c3ccc4sc5c(N(c6ccc(-c7ccccc7)cc6)c6cc(-c7ccccc7)cc(-c7ccccc7)c6)cc6ccccc6c5c4c3)c21]

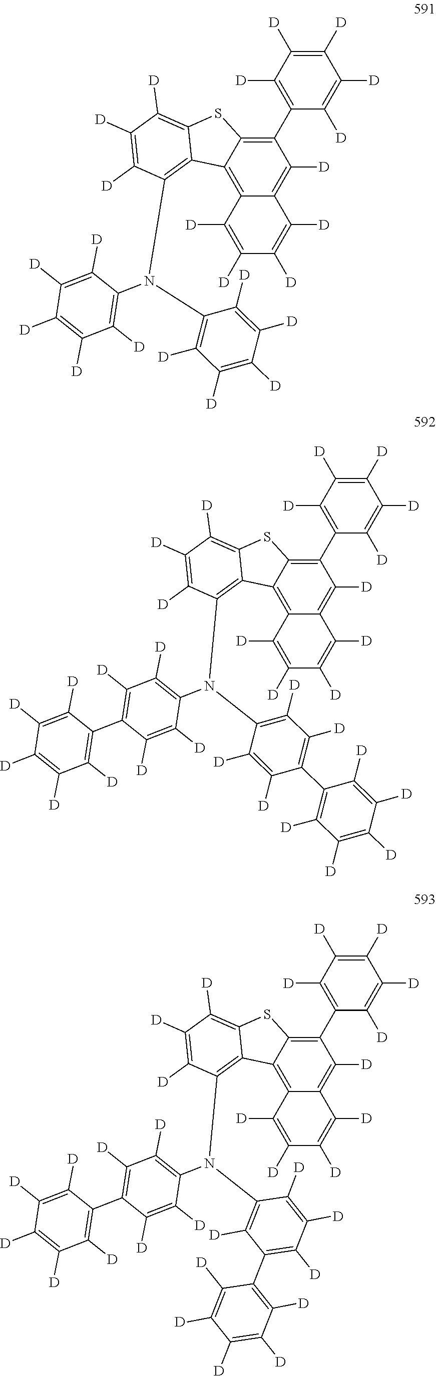

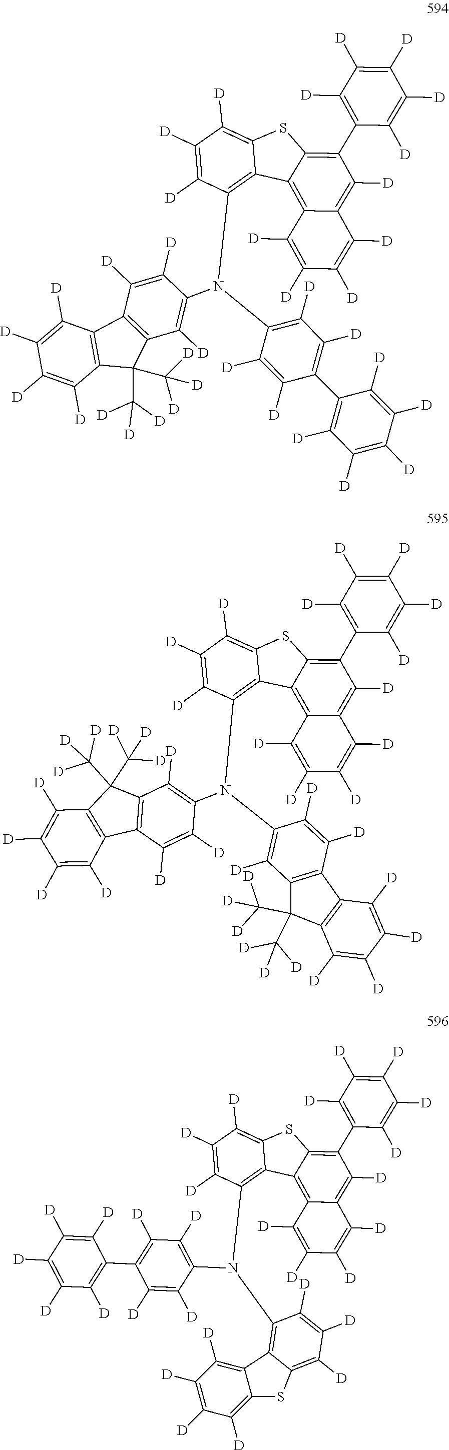

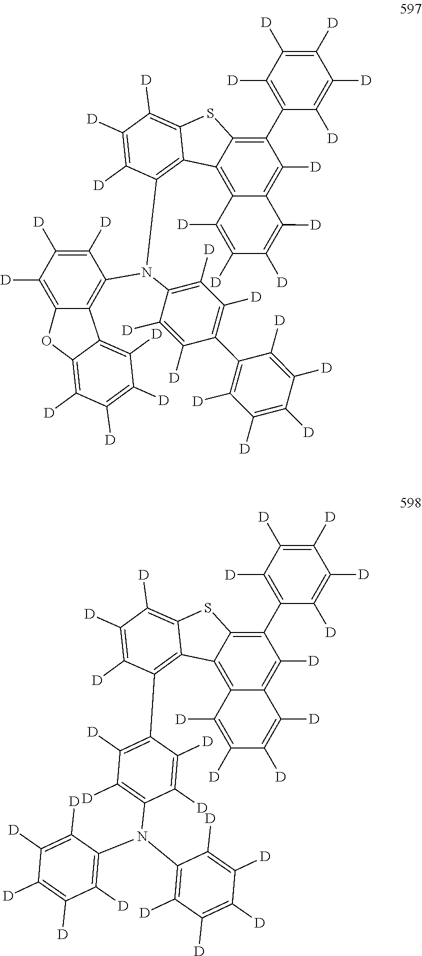 [SMILES: [2H]c1c([2H])c([2H])c(-c2c([2H])c([2H])c(N(c3c([2H])c([2H])c([2H])c4oc5c([2H])c([2H])c([2H])c([2H])c5c34)c3c([2H])c([2H])c([2H])c4sc5c(-c6c([2H])c([2H])c([2H])c([2H])c6[2H])c([2H])c6c([2H])c([2H])c([2H])c([2H])c6c5c34)c([2H])c2[2H])c([2H])c1[2H] ; [2H]c1c([2H])c([2H])c(-c2c([2H])c3c([2H])c([2H])c([2H])c([2H])c3c3c2sc2c([2H])c([2H])c([2H])c(-c4c([2H])c([2H])c(N(c5c([2H])c([2H])c([2H])c([2H])c5[2H])c5c([2H])c([2H])c([2H])c([2H])c5[2H])c([2H])c4[2H])c23)c([2H])c1[2H]]

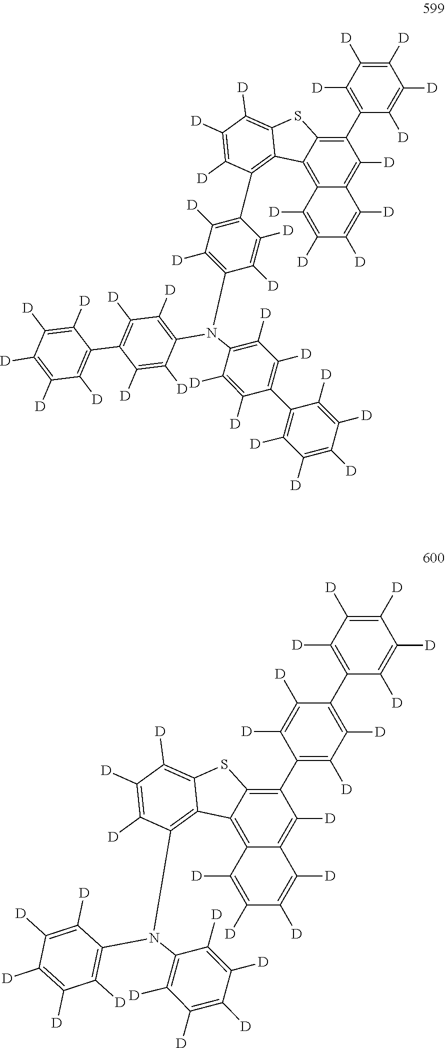

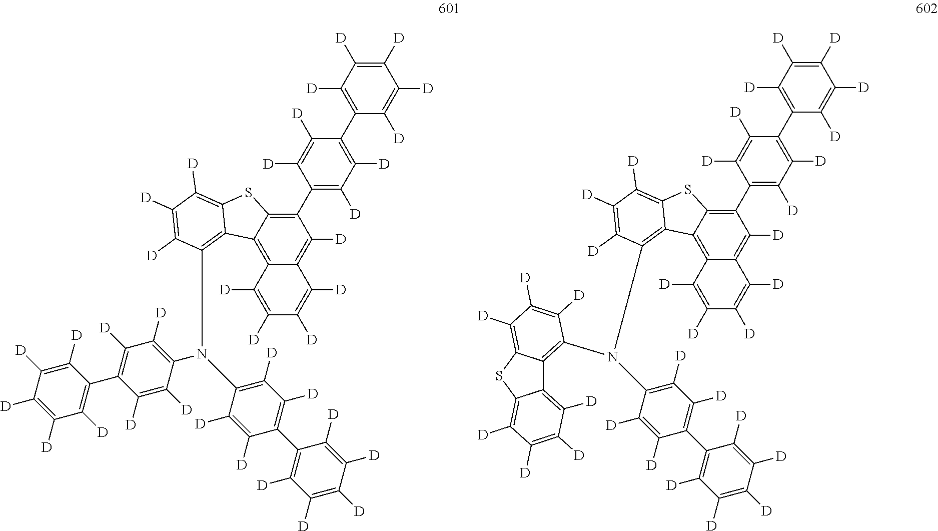

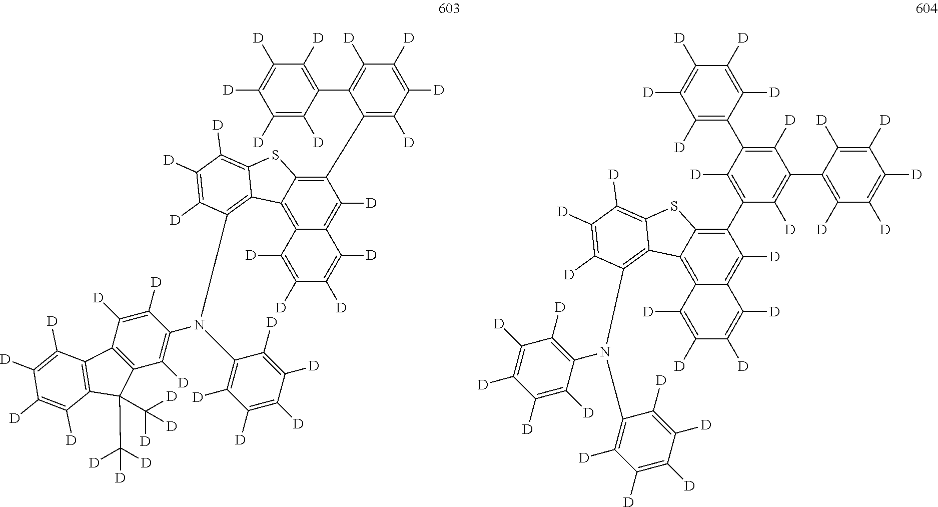

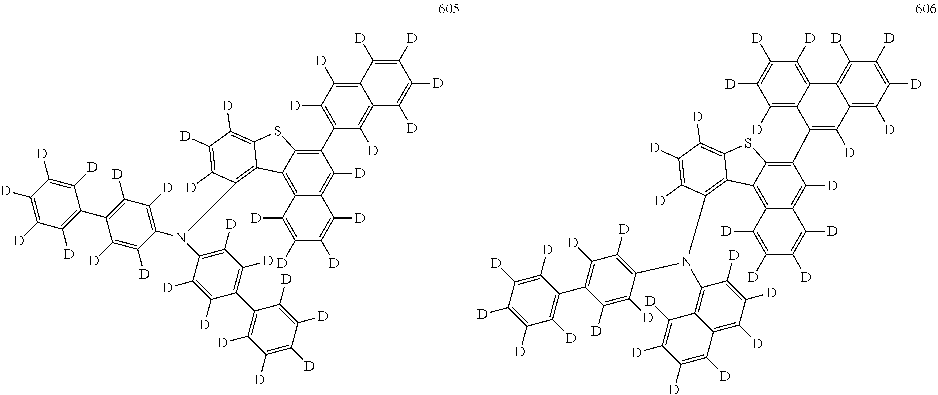

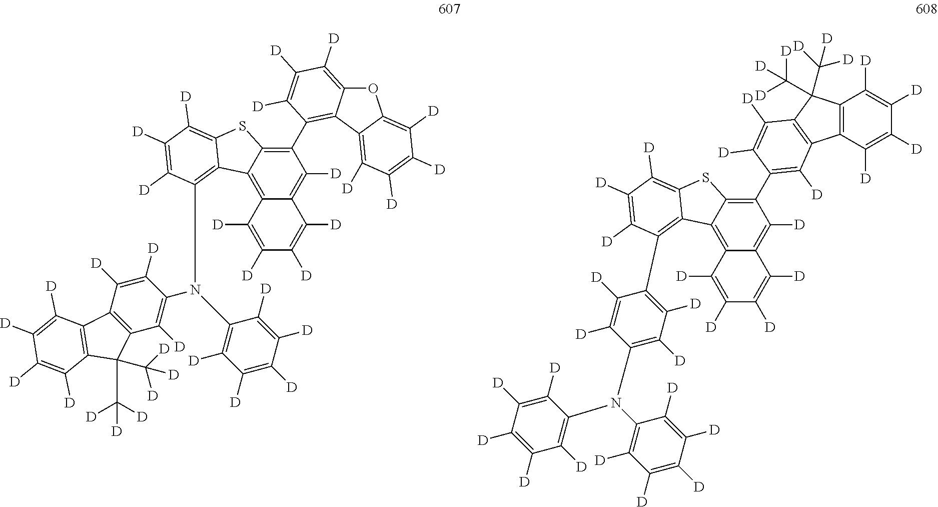

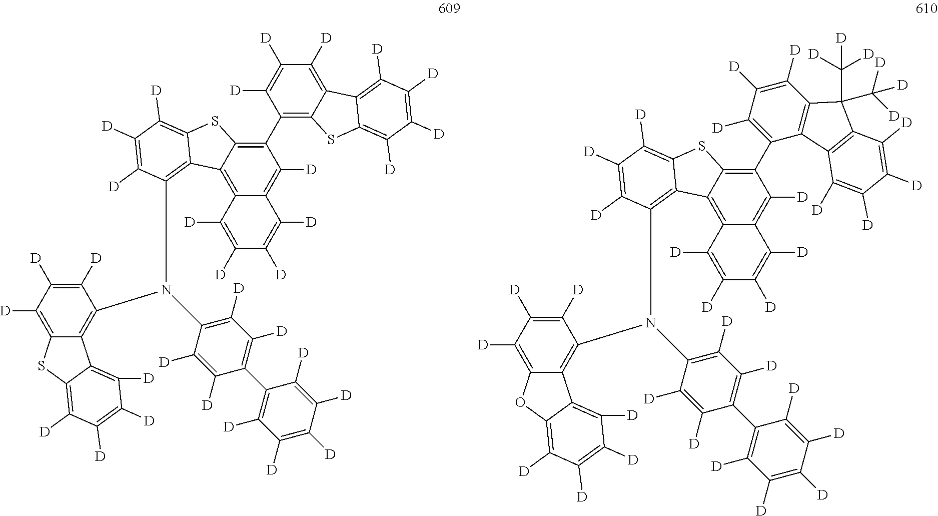

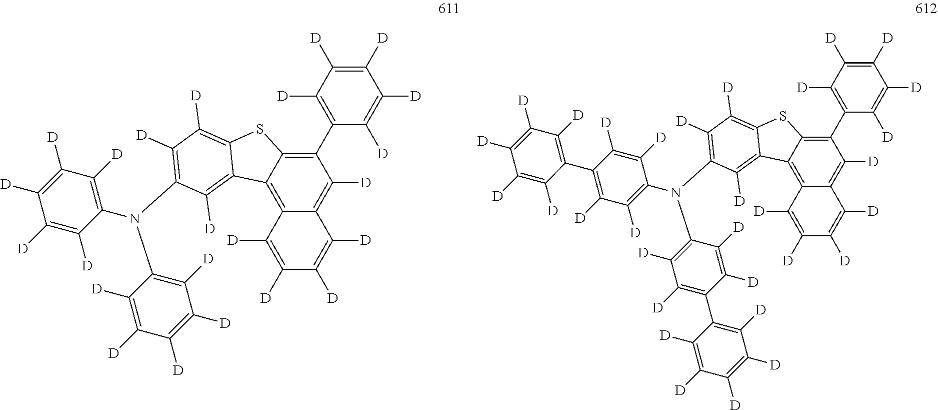 [SMILES: [2H]c1c([2H])c([2H])c(-c2c([2H])c3c([2H])c([2H])c([2H])c([2H])c3c3c2sc2c([2H])c([2H])c(N(c4c([2H])c([2H])c([2H])c([2H])c4[2H])c4c([2H])c([2H])c([2H])c([2H])c4[2H])c([2H])c23)c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4sc5c(-c6c([2H])c([2H])c([2H])c([2H])c6[2H])c([2H])c6c([2H])c([2H])c([2H])c([2H])c6c5c4c3[2H])c([2H])c2[2H])c([2H])c1[2H]]

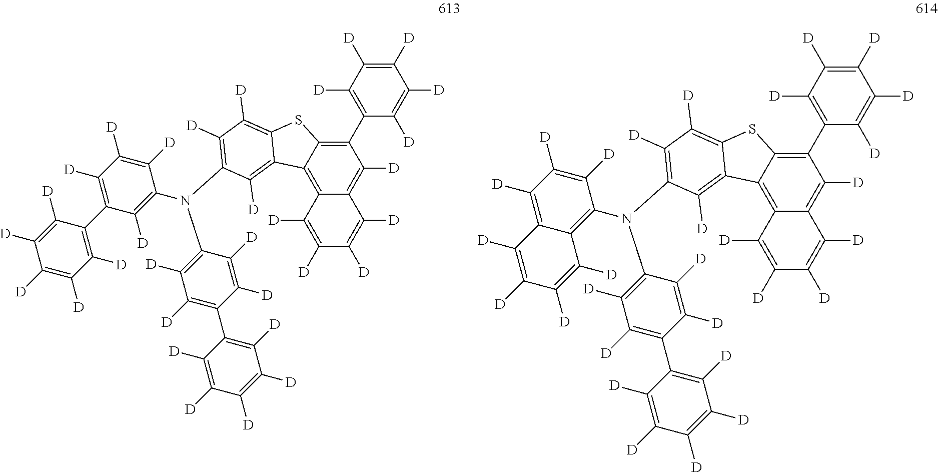 [SMILES: [2H]c1c([2H])c([2H])c(-c2c([2H])c([2H])c(N(c3c([2H])c([2H])c([2H])c(-c4c([2H])c([2H])c([2H])c([2H])c4[2H])c3[2H])c3c([2H])c([2H])c4sc5c(-c6c([2H])c([2H])c([2H])c([2H])c6[2H])c([2H])c6c([2H])c([2H])c([2H])c([2H])c6c5c4c3[2H])c([2H])c2[2H])c([2H])c1[2H] ; [2H]c1c([2H])c([2H])c(-c2c([2H])c([2H])c(N(c3c([2H])c([2H])c4sc5c(-c6c([2H])c([2H])c([2H])c([2H])c6[2H])c([2H])c6c([2H])c([2H])c([2H])c([2H])c6c5c4c3[2H])c3c([2H])c([2H])c([2H])c4c([2H])c([2H])c([2H])c([2H])c34)c([2H])c2[2H])c([2H])c1[2H]]

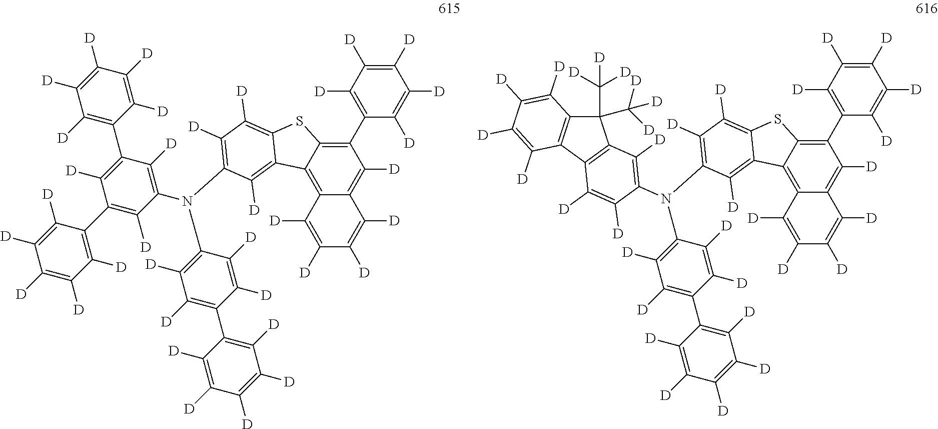

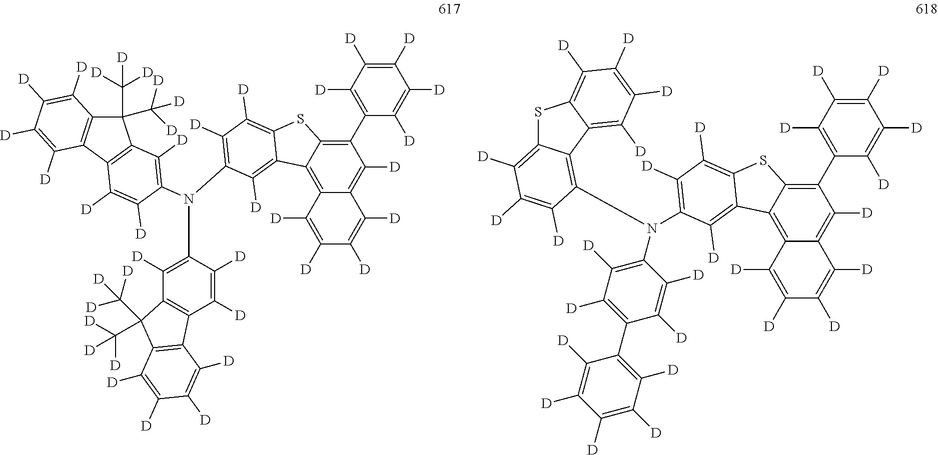

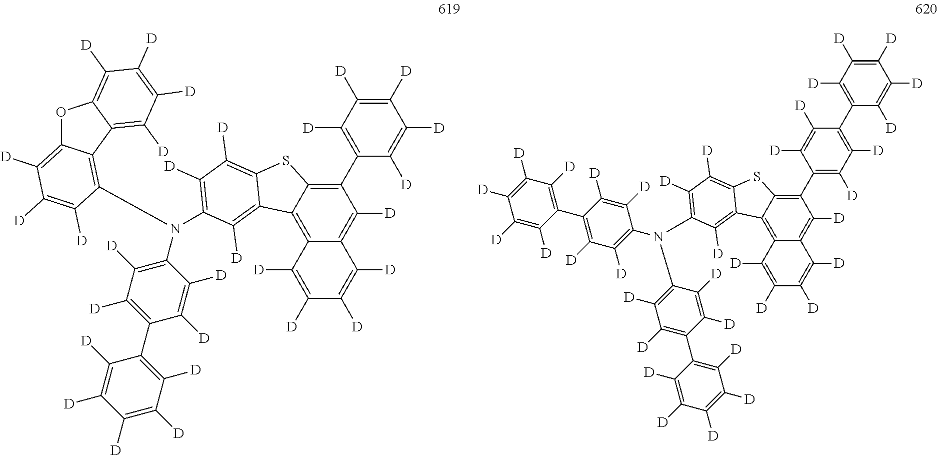

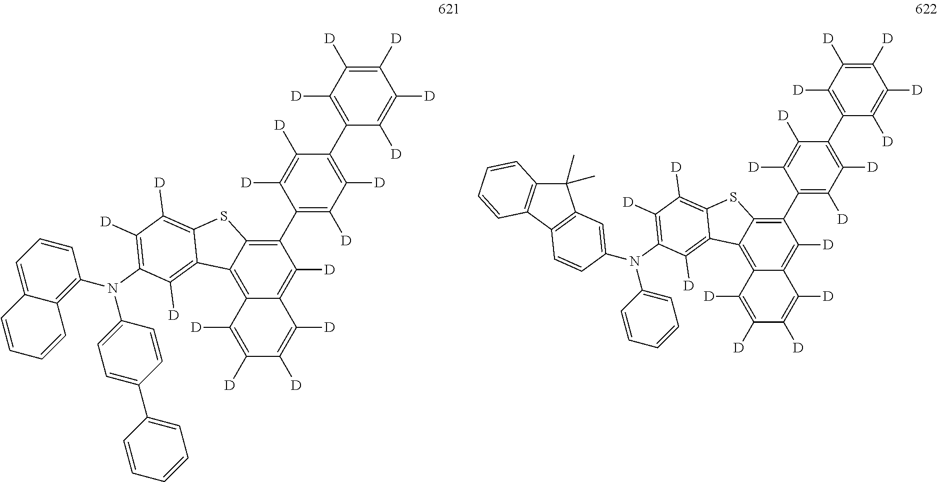 [SMILES: [2H]c1c([2H])c([2H])c(-c2c([2H])c([2H])c(-c3c([2H])c4c([2H])c([2H])c([2H])c([2H])c4c4c3sc3c([2H])c([2H])c(N(c5ccc(-c6ccccc6)cc5)c5cccc6ccccc56)c([2H])c34)c([2H])c2[2H])c([2H])c1[2H] ; [2H]c1c([2H])c([2H])c(-c2c([2H])c([2H])c(-c3c([2H])c4c([2H])c([2H])c([2H])c([2H])c4c4c3sc3c([2H])c([2H])c(N(c5ccccc5)c5ccc6c(c5)C(C)(C)c5ccccc5-6)c([2H])c34)c([2H])c2[2H])c([2H])c1[2H]]

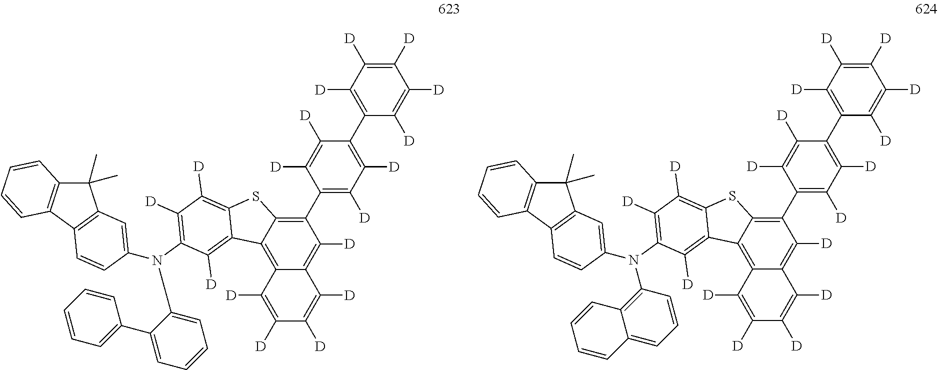 [SMILES: [2H]c1c([2H])c([2H])c(-c2c([2H])c([2H])c(-c3c([2H])c4c([2H])c([2H])c([2H])c([2H])c4c4c3sc3c([2H])c([2H])c(N(c5ccc6c(c5)C(C)(C)c5ccccc5-6)c5ccccc5-c5ccccc5)c([2H])c34)c([2H])c2[2H])c([2H])c1[2H] ; [2H]c1c([2H])c([2H])c(-c2c([2H])c([2H])c(-c3c([2H])c4c([2H])c([2H])c([2H])c([2H])c4c4c3sc3c([2H])c([2H])c(N(c5ccc6c(c5)C(C)(C)c5ccccc5-6)c5cccc6ccccc56)c([2H])c34)c([2H])c2[2H])c([2H])c1[2H]]

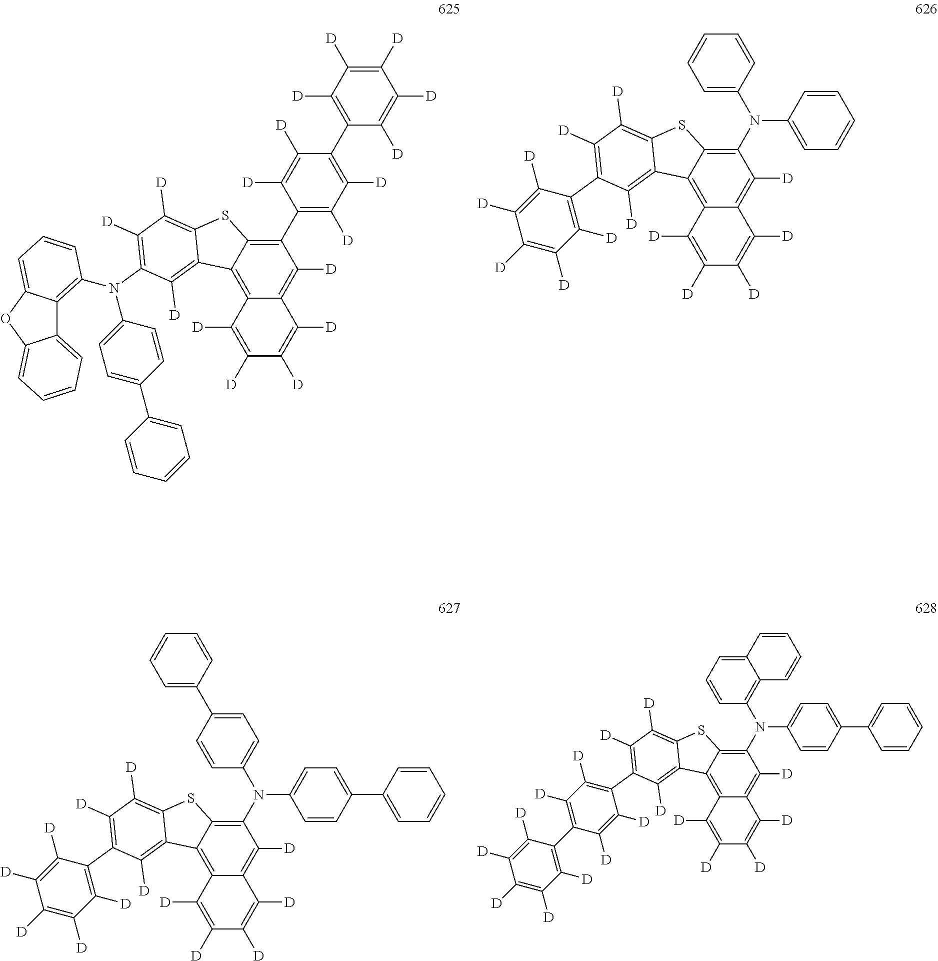

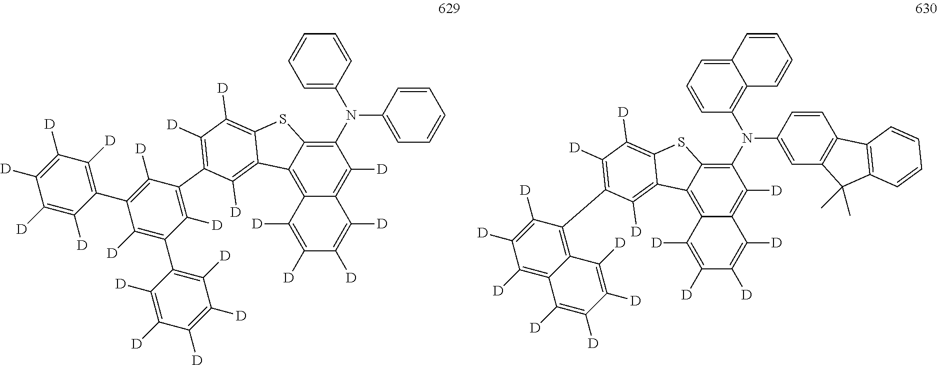 [SMILES: [2H]c1c([2H])c([2H])c(-c2c([2H])c(-c3c([2H])c([2H])c([2H])c([2H])c3[2H])c([2H])c(-c3c([2H])c([2H])c4sc5c(N(c6ccccc6)c6ccccc6)c([2H])c6c([2H])c([2H])c([2H])c([2H])c6c5c4c3[2H])c2[2H])c([2H])c1[2H] ; [2H]c1c(-c2c([2H])c([2H])c([2H])c3c([2H])c([2H])c([2H])c([2H])c23)c([2H])c2c(sc3c(N(c4ccc5c(c4)C(C)(C)c4ccccc4-5)c4cccc5ccccc45)c([2H])c4c([2H])c([2H])c([2H])c([2H])c4c32)c1[2H]]

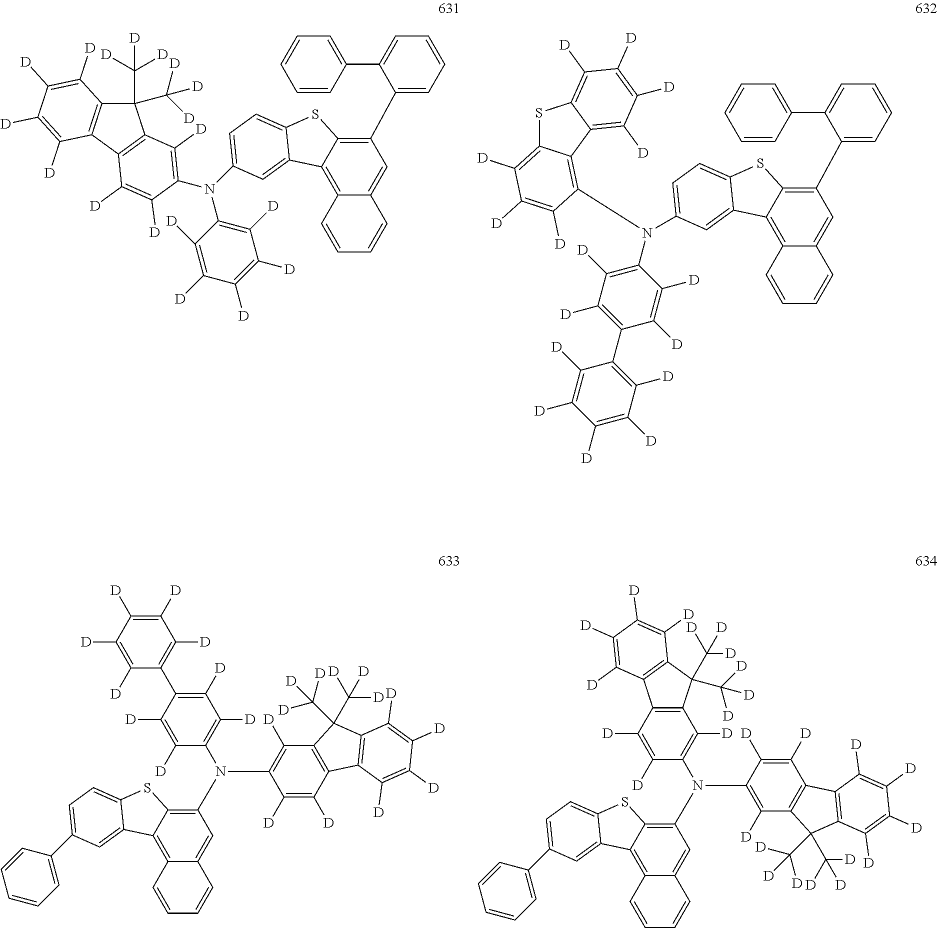

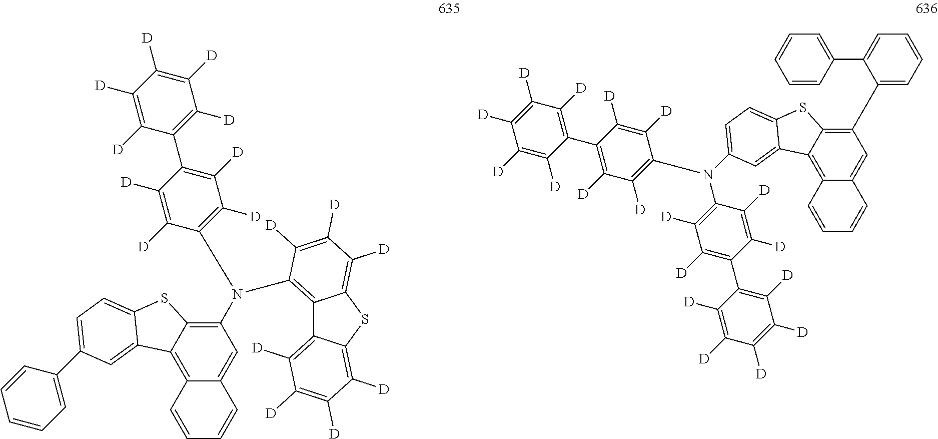 [SMILES: [2H]c1c([2H])c([2H])c(-c2c([2H])c([2H])c(N(c3cc4ccccc4c4c3sc3ccc(-c5ccccc5)cc34)c3c([2H])c([2H])c([2H])c4sc5c([2H])c([2H])c([2H])c([2H])c5c34)c([2H])c2[2H])c([2H])c1[2H] ; [2H]c1c([2H])c([2H])c(-c2c([2H])c([2H])c(N(c3ccc4sc5c(-c6ccccc6-c6ccccc6)cc6ccccc6c5c4c3)c3c([2H])c([2H])c(-c4c([2H])c([2H])c([2H])c([2H])c4[2H])c([2H])c3[2H])c([2H])c2[2H])c([2H])c1[2H]]

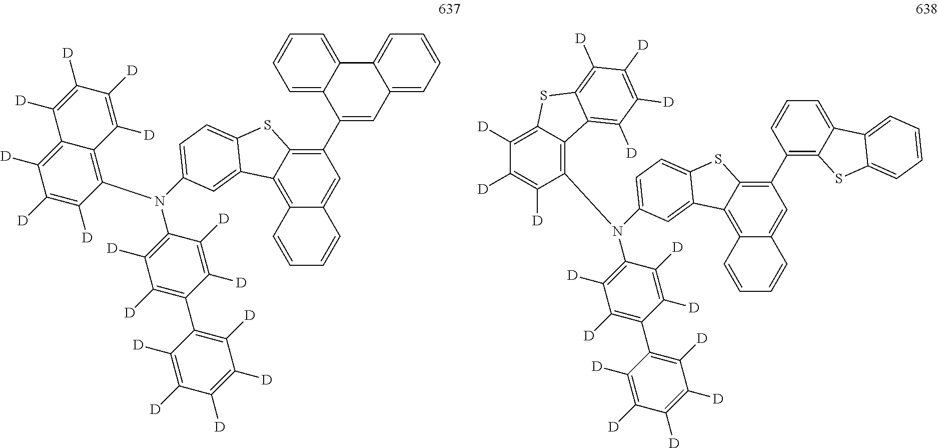 [SMILES: [2H]c1c([2H])c([2H])c(-c2c([2H])c([2H])c(N(c3ccc4sc5c(-c6cc7ccccc7c7ccccc67)cc6ccccc6c5c4c3)c3c([2H])c([2H])c([2H])c4c([2H])c([2H])c([2H])c([2H])c34)c([2H])c2[2H])c([2H])c1[2H] ; [2H]c1c([2H])c([2H])c(-c2c([2H])c([2H])c(N(c3ccc4sc5c(-c6cccc7c6sc6ccccc67)cc6ccccc6c5c4c3)c3c([2H])c([2H])c([2H])c4sc5c([2H])c([2H])c([2H])c([2H])c5c34)c([2H])c2[2H])c([2H])c1[2H]]

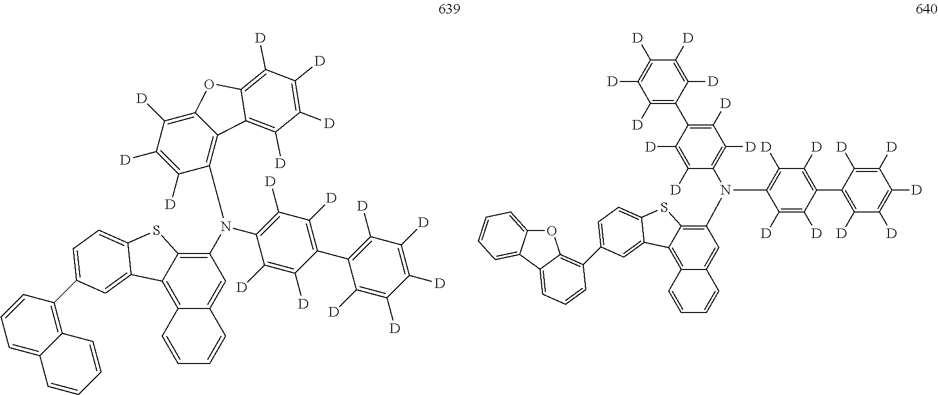 [SMILES: [2H]c1c([2H])c([2H])c(-c2c([2H])c([2H])c(N(c3cc4ccccc4c4c3sc3ccc(-c5cccc6ccccc56)cc34)c3c([2H])c([2H])c([2H])c4oc5c([2H])c([2H])c([2H])c([2H])c5c34)c([2H])c2[2H])c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4ccccc4c4c3sc3ccc(-c5cccc6c5oc5ccccc56)cc34)c([2H])c2[2H])c([2H])c1[2H]]

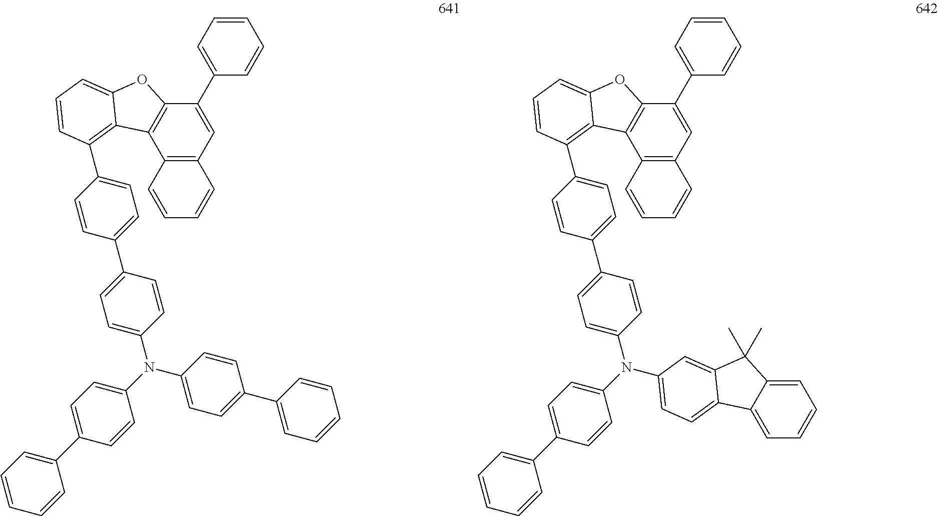 [SMILES: c1ccc(-c2ccc(N(c3ccc(-c4ccccc4)cc3)c3ccc(-c4ccc(-c5cccc6oc7c(-c8ccccc8)cc8ccccc8c7c56)cc4)cc3)cc2)cc1 ; CC1(C)c2ccccc2-c2ccc(N(c3ccc(-c4ccccc4)cc3)c3ccc(-c4ccc(-c5cccc6oc7c(-c8ccccc8)cc8ccccc8c7c56)cc4)cc3)cc21]

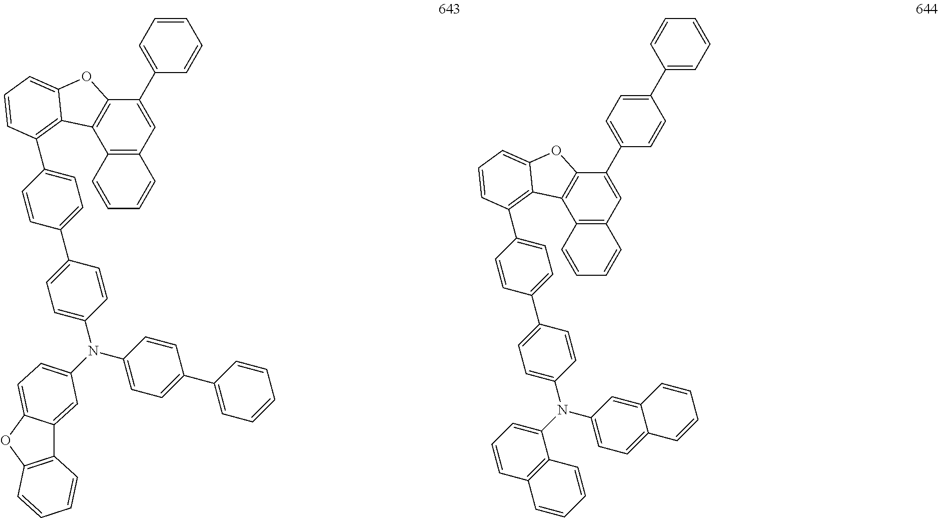 [SMILES: c1ccc(-c2ccc(N(c3ccc(-c4ccc(-c5cccc6oc7c(-c8ccccc8)cc8ccccc8c7c56)cc4)cc3)c3ccc4oc5ccccc5c4c3)cc2)cc1 ; c1ccc(-c2ccc(-c3cc4ccccc4c4c3oc3cccc(-c5ccc(-c6ccc(N(c7ccc8ccccc8c7)c7cccc8ccccc78)cc6)cc5)c34)cc2)cc1]

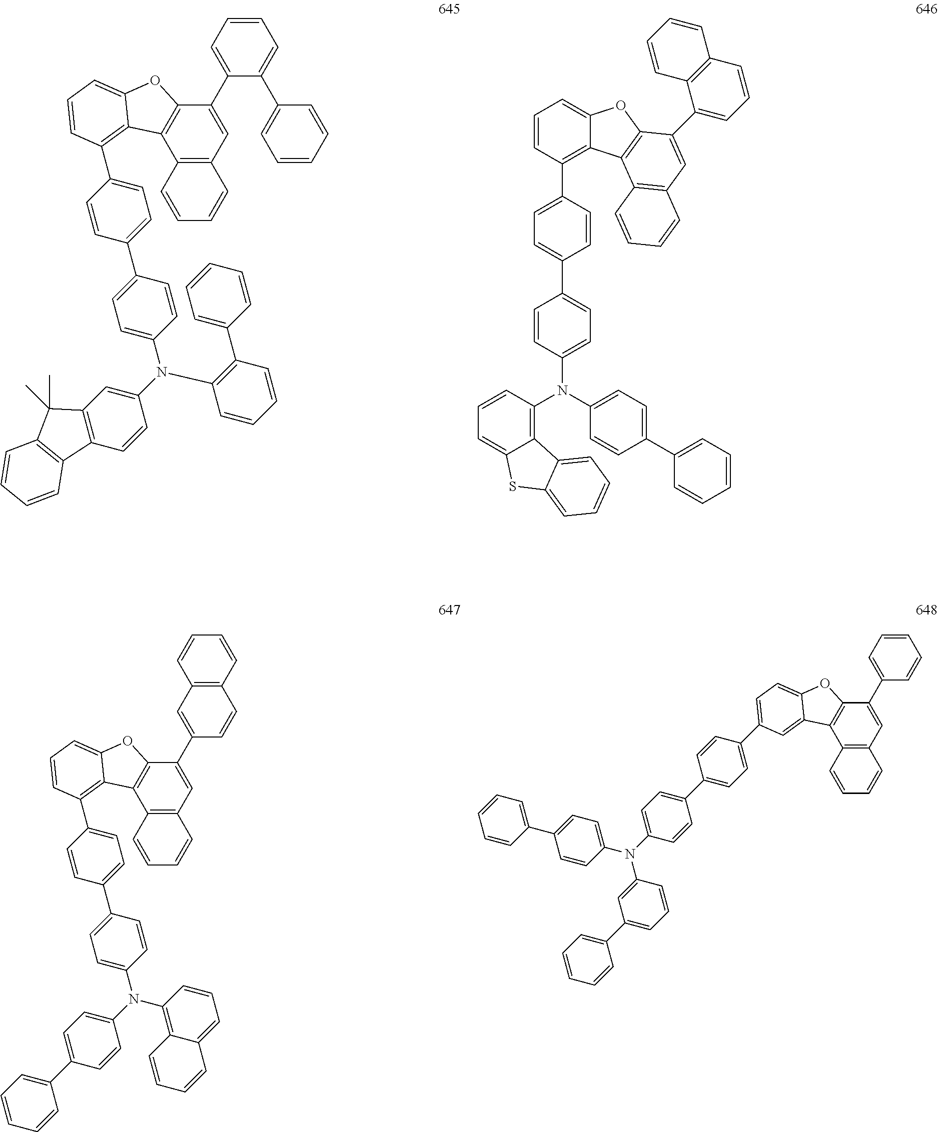 [SMILES: CC1(C)c2ccccc2-c2ccc(N(c3ccc(-c4ccc(-c5cccc6oc7c(-c8ccccc8-c8ccccc8)cc8ccccc8c7c56)cc4)cc3)c3ccccc3-c3ccccc3)cc21 ; c1ccc(-c2ccc(N(c3ccc(-c4ccc(-c5cccc6oc7c(-c8cccc9ccccc89)cc8ccccc8c7c56)cc4)cc3)c3cccc4sc5ccccc5c34)cc2)cc1 ; c1ccc(-c2ccc(N(c3ccc(-c4ccc(-c5cccc6oc7c(-c8ccc9ccccc9c8)cc8ccccc8c7c56)cc4)cc3)c3cccc4ccccc34)cc2)cc1 ; c1ccc(-c2ccc(N(c3ccc(-c4ccc(-c5ccc6oc7c(-c8ccccc8)cc8ccccc8c7c6c5)cc4)cc3)c3cccc(-c4ccccc4)c3)cc2)cc1]

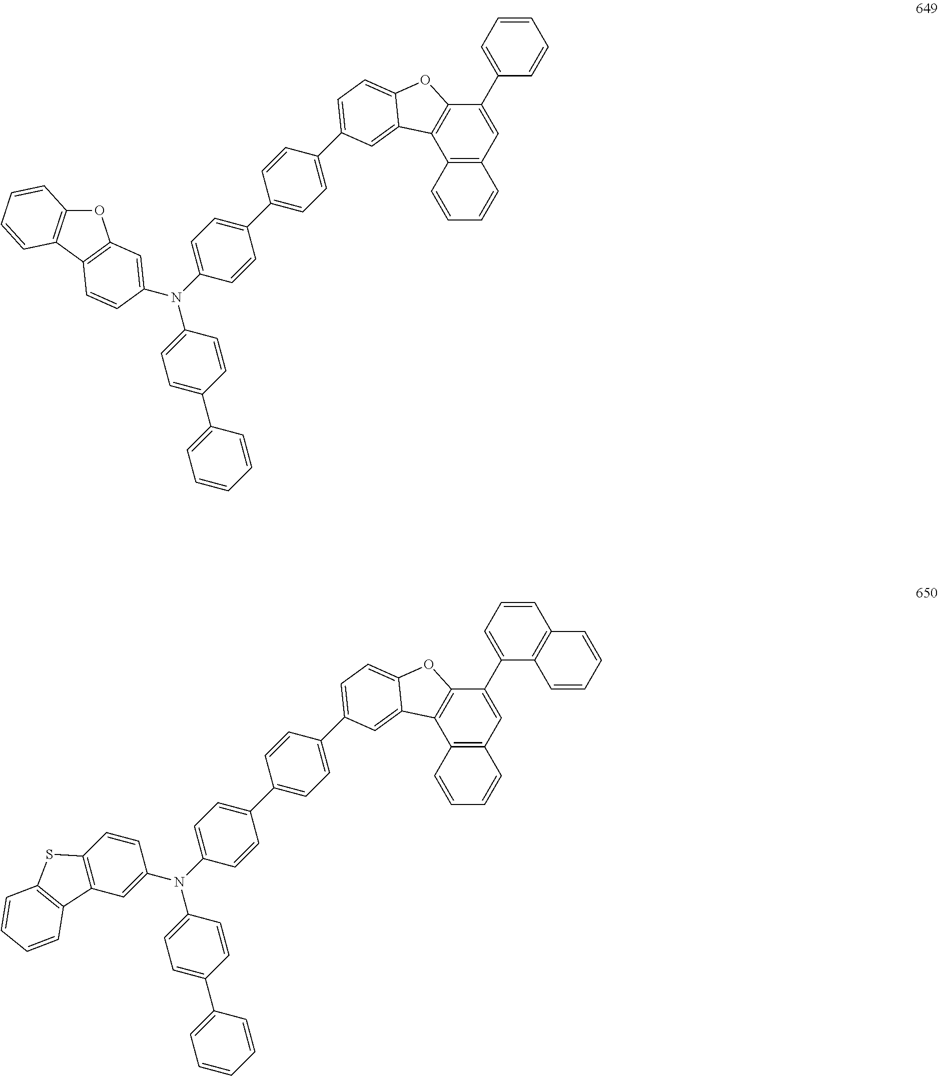 [SMILES: c1ccc(-c2ccc(N(c3ccc(-c4ccc(-c5ccc6oc7c(-c8ccccc8)cc8ccccc8c7c6c5)cc4)cc3)c3ccc4c(c3)oc3ccccc34)cc2)cc1 ; c1ccc(-c2ccc(N(c3ccc(-c4ccc(-c5ccc6oc7c(-c8cccc9ccccc89)cc8ccccc8c7c6c5)cc4)cc3)c3ccc4sc5ccccc5c4c3)cc2)cc1]

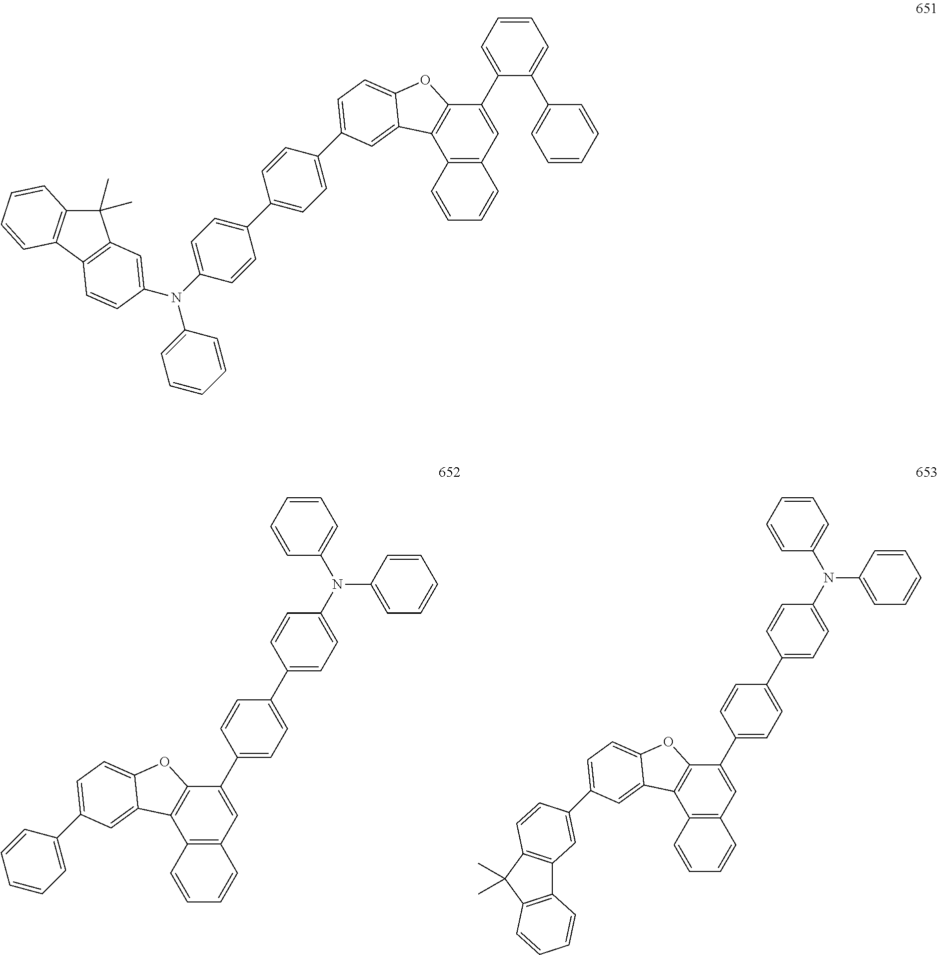 [SMILES: CC1(C)c2ccccc2-c2ccc(N(c3ccccc3)c3ccc(-c4ccc(-c5ccc6oc7c(-c8ccccc8-c8ccccc8)cc8ccccc8c7c6c5)cc4)cc3)cc21 ; c1ccc(-c2ccc3oc4c(-c5ccc(-c6ccc(N(c7ccccc7)c7ccccc7)cc6)cc5)cc5ccccc5c4c3c2)cc1 ; CC1(C)c2ccccc2-c2cc(-c3ccc4oc5c(-c6ccc(-c7ccc(N(c8ccccc8)c8ccccc8)cc7)cc6)cc6ccccc6c5c4c3)ccc21]

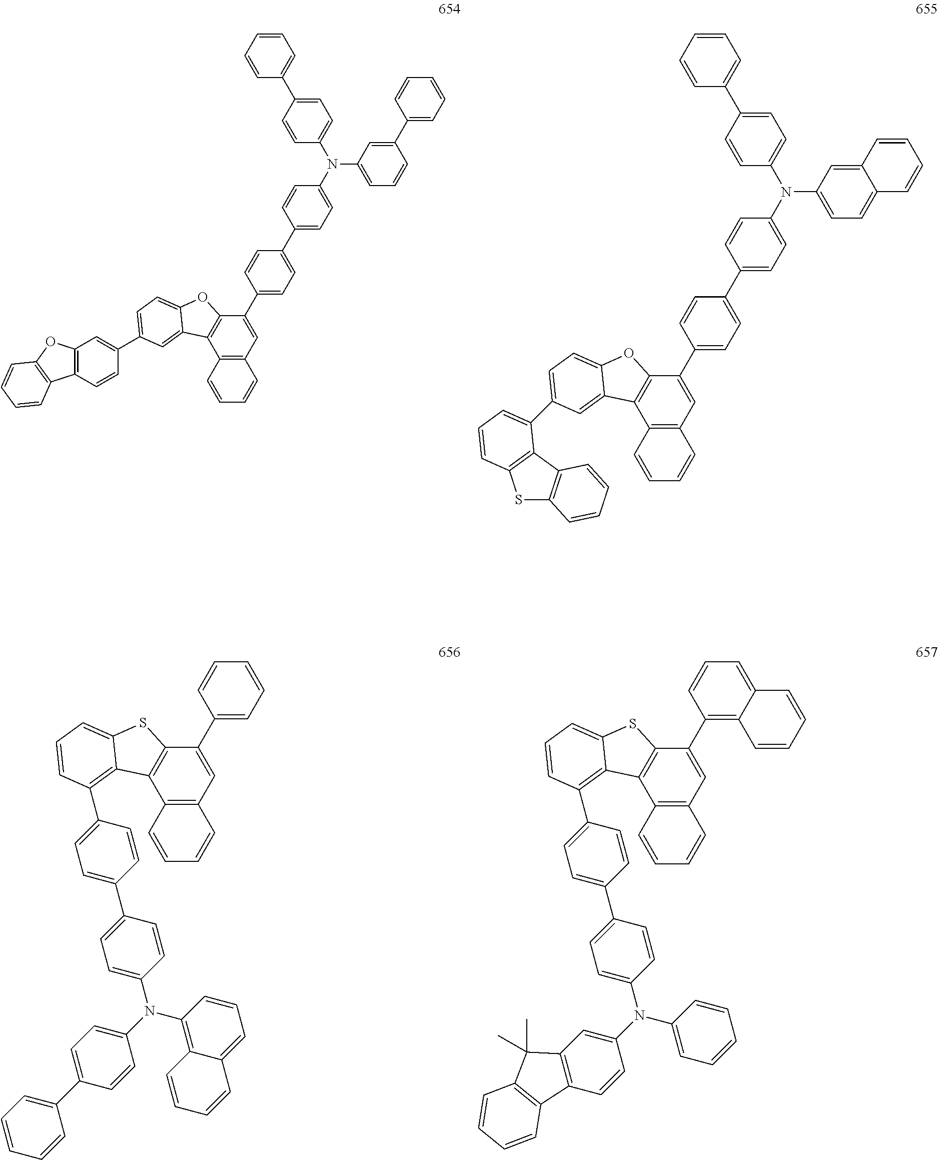 [SMILES: c1ccc(-c2ccc(N(c3ccc(-c4ccc(-c5cc6ccccc6c6c5oc5ccc(-c7ccc8c(c7)oc7ccccc78)cc56)cc4)cc3)c3cccc(-c4ccccc4)c3)cc2)cc1 ; c1ccc(-c2ccc(N(c3ccc(-c4ccc(-c5cc6ccccc6c6c5oc5ccc(-c7cccc8sc9ccccc9c78)cc56)cc4)cc3)c3ccc4ccccc4c3)cc2)cc1 ; c1ccc(-c2ccc(N(c3ccc(-c4ccc(-c5cccc6sc7c(-c8ccccc8)cc8ccccc8c7c56)cc4)cc3)c3cccc4ccccc34)cc2)cc1 ; CC1(C)c2ccccc2-c2ccc(N(c3ccccc3)c3ccc(-c4ccc(-c5cccc6sc7c(-c8cccc9ccccc89)cc8ccccc8c7c56)cc4)cc3)cc21]

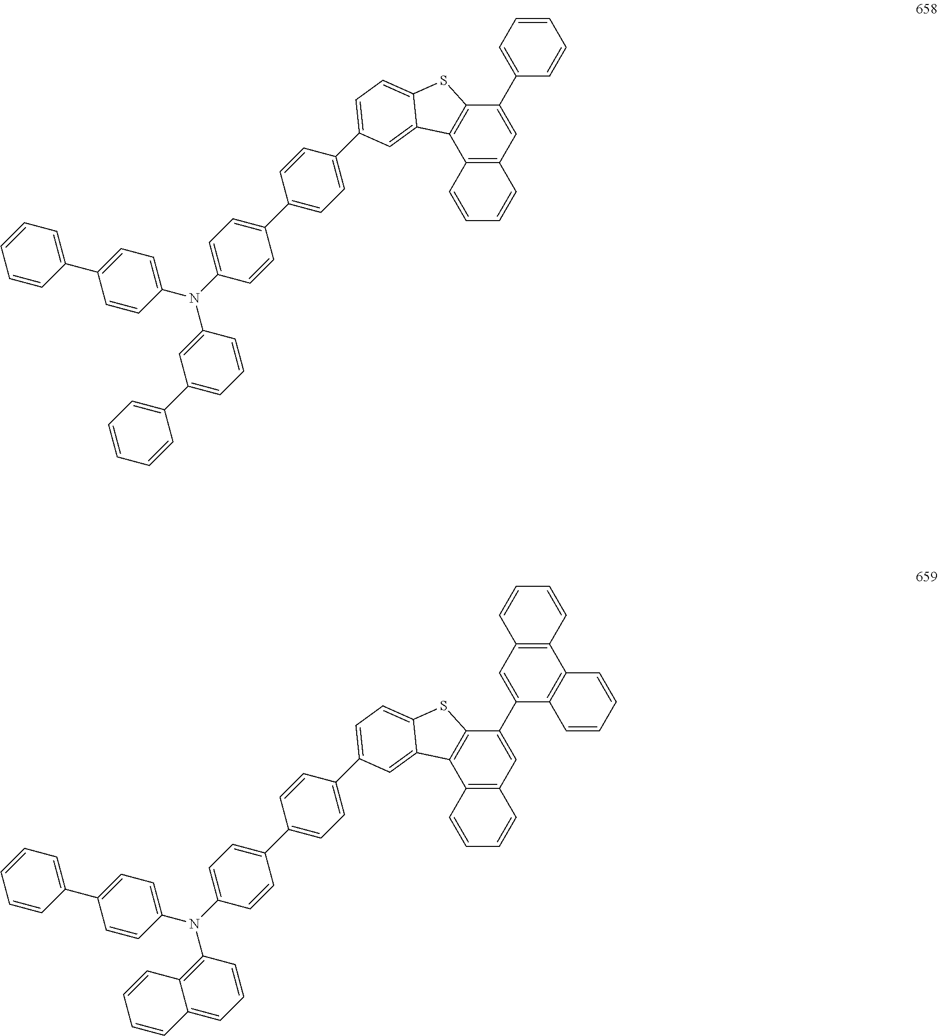 [SMILES: c1ccc(-c2ccc(N(c3ccc(-c4ccc(-c5ccc6sc7c(-c8ccccc8)cc8ccccc8c7c6c5)cc4)cc3)c3cccc(-c4ccccc4)c3)cc2)cc1 ; c1ccc(-c2ccc(N(c3ccc(-c4ccc(-c5ccc6sc7c(-c8cc9ccccc9c9ccccc89)cc8ccccc8c7c6c5)cc4)cc3)c3cccc4ccccc34)cc2)cc1]

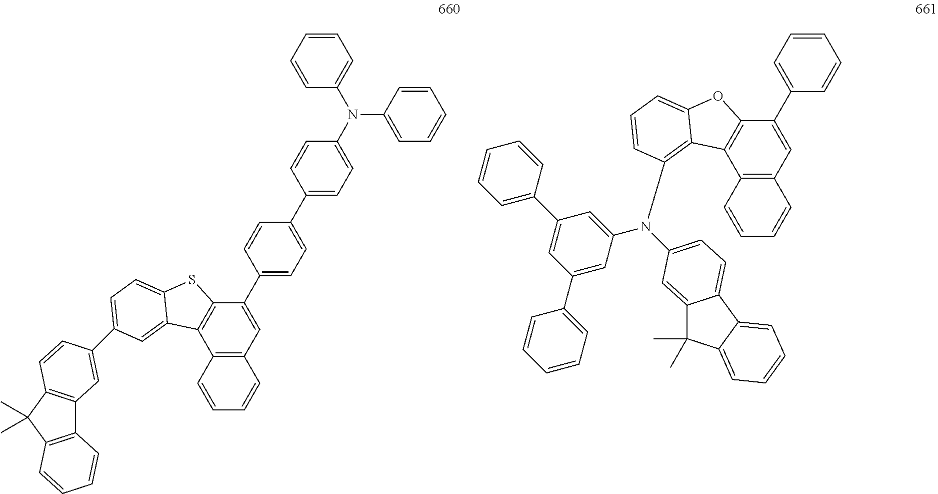 [SMILES: CC1(C)c2ccccc2-c2cc(-c3ccc4sc5c(-c6ccc(-c7ccc(N(c8ccccc8)c8ccccc8)cc7)cc6)cc6ccccc6c5c4c3)ccc21 ; CC1(C)c2ccccc2-c2ccc(N(c3cc(-c4ccccc4)cc(-c4ccccc4)c3)c3cccc4oc5c(-c6ccccc6)cc6ccccc6c5c34)cc21]

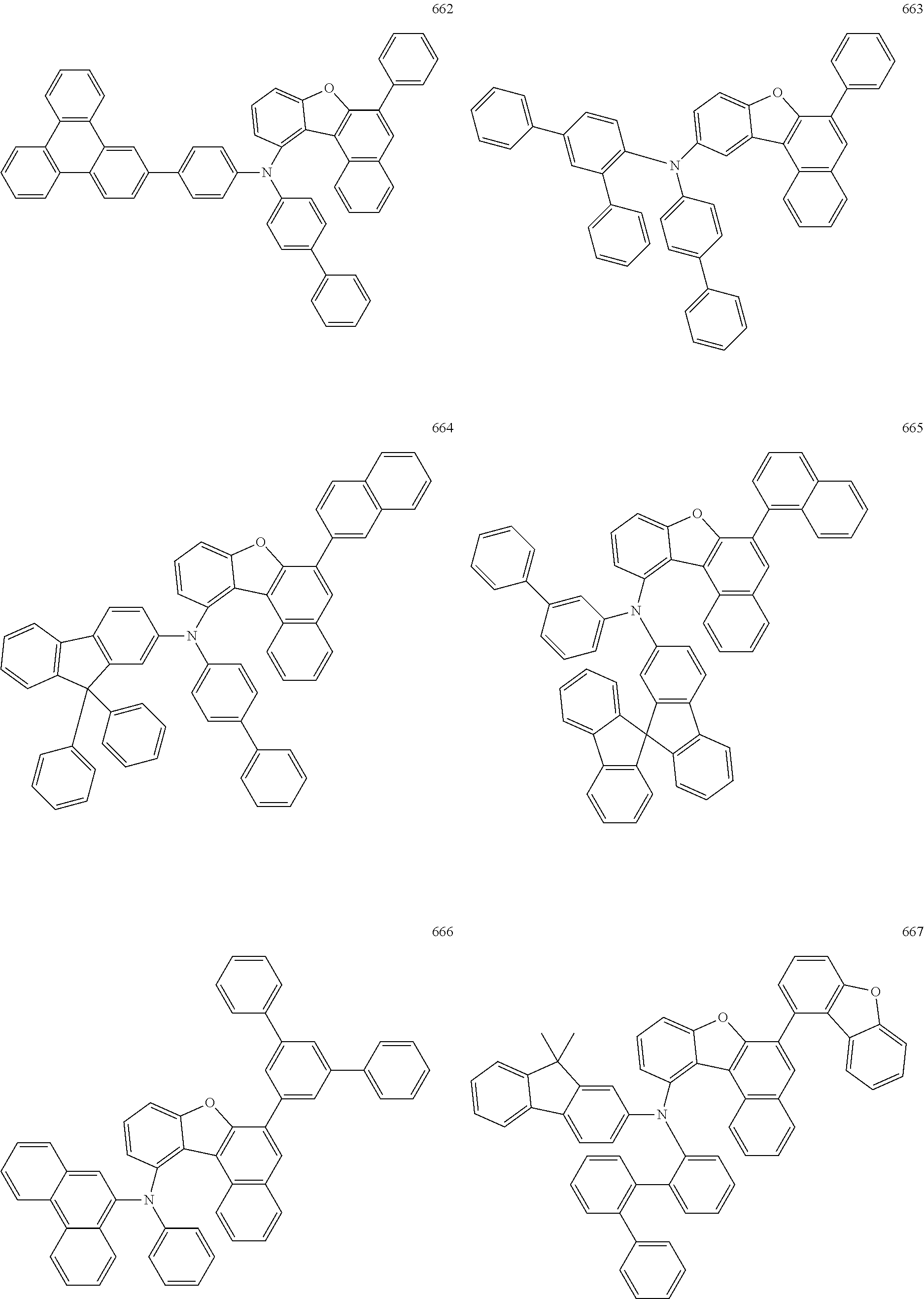

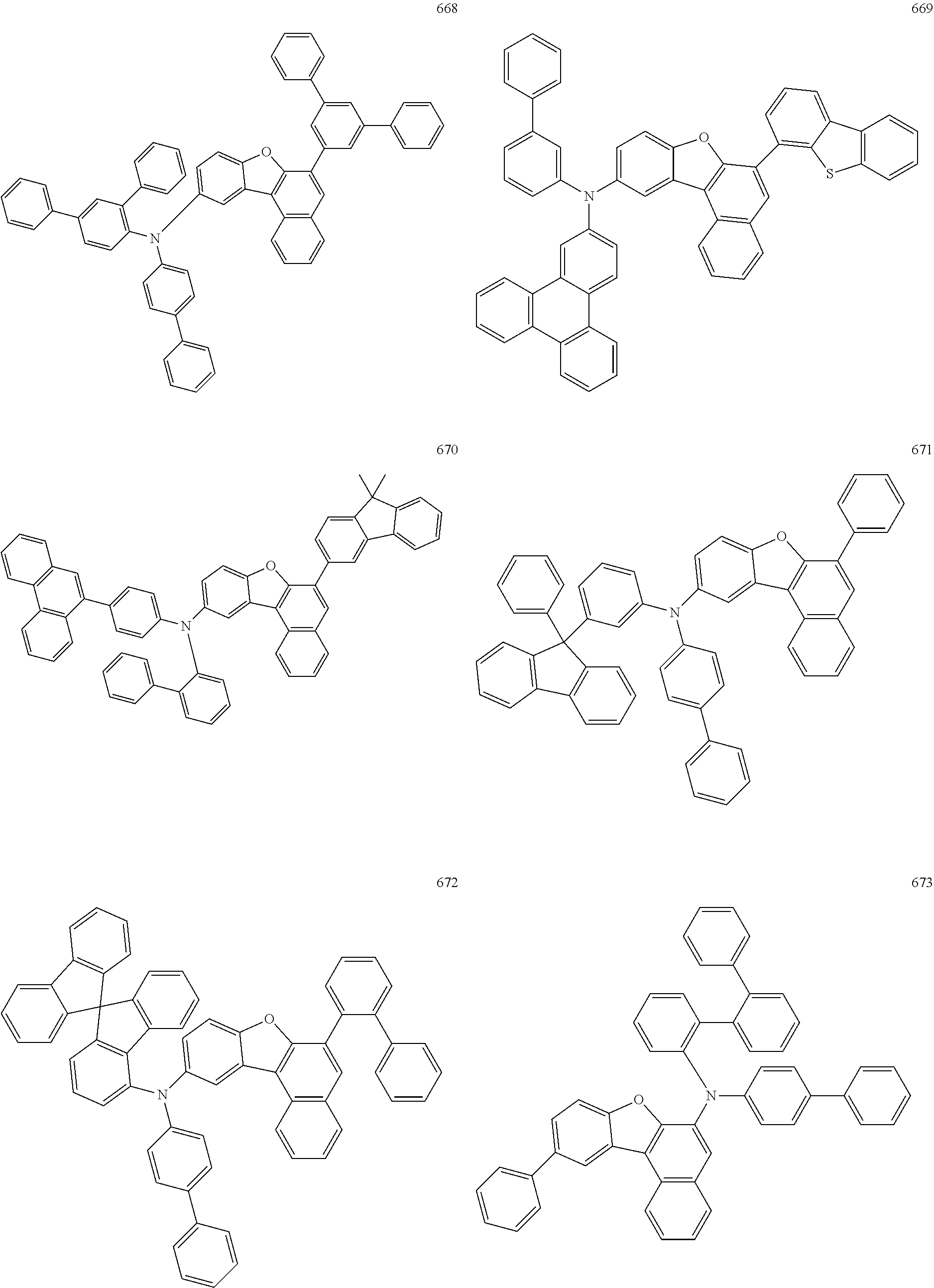

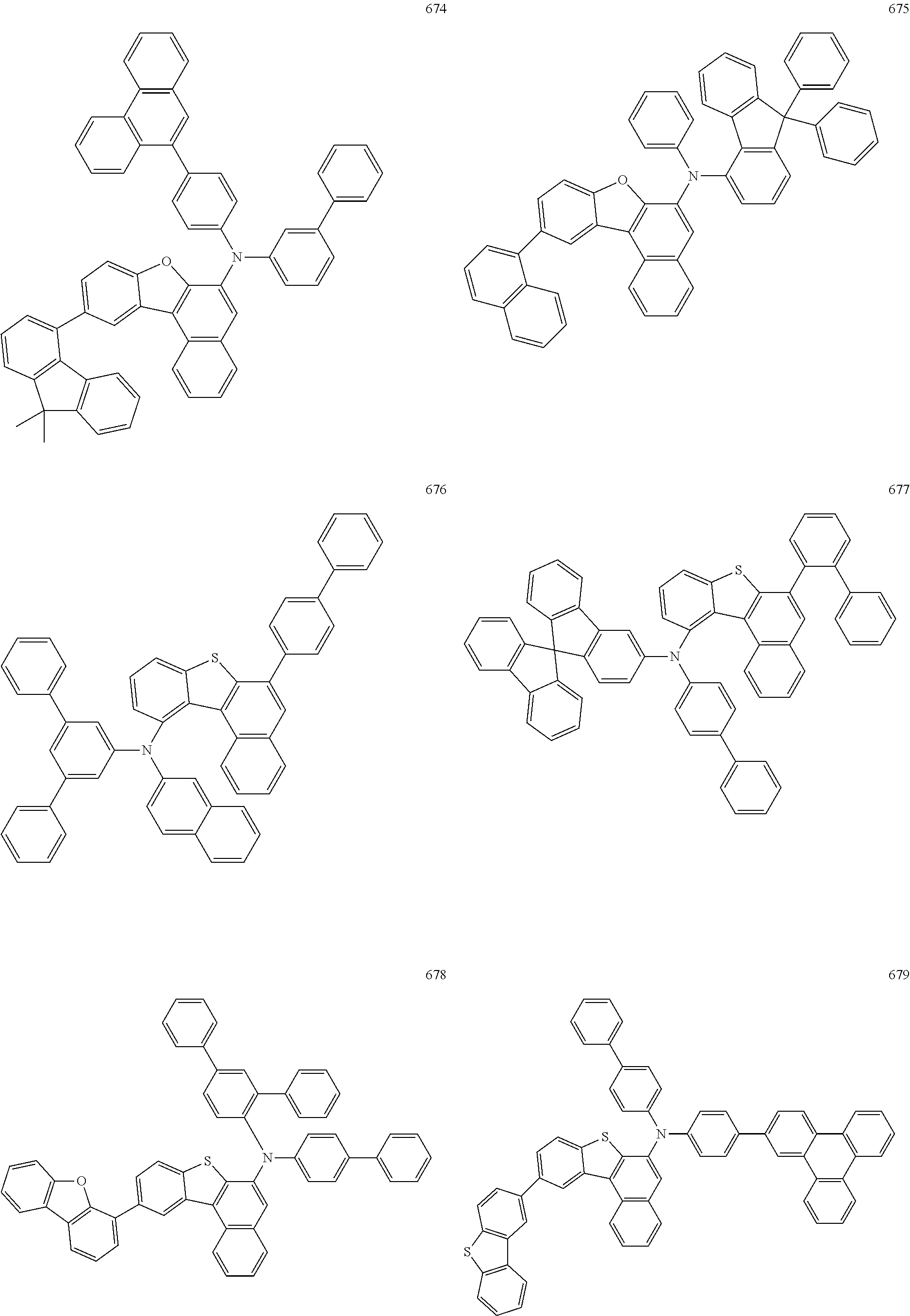

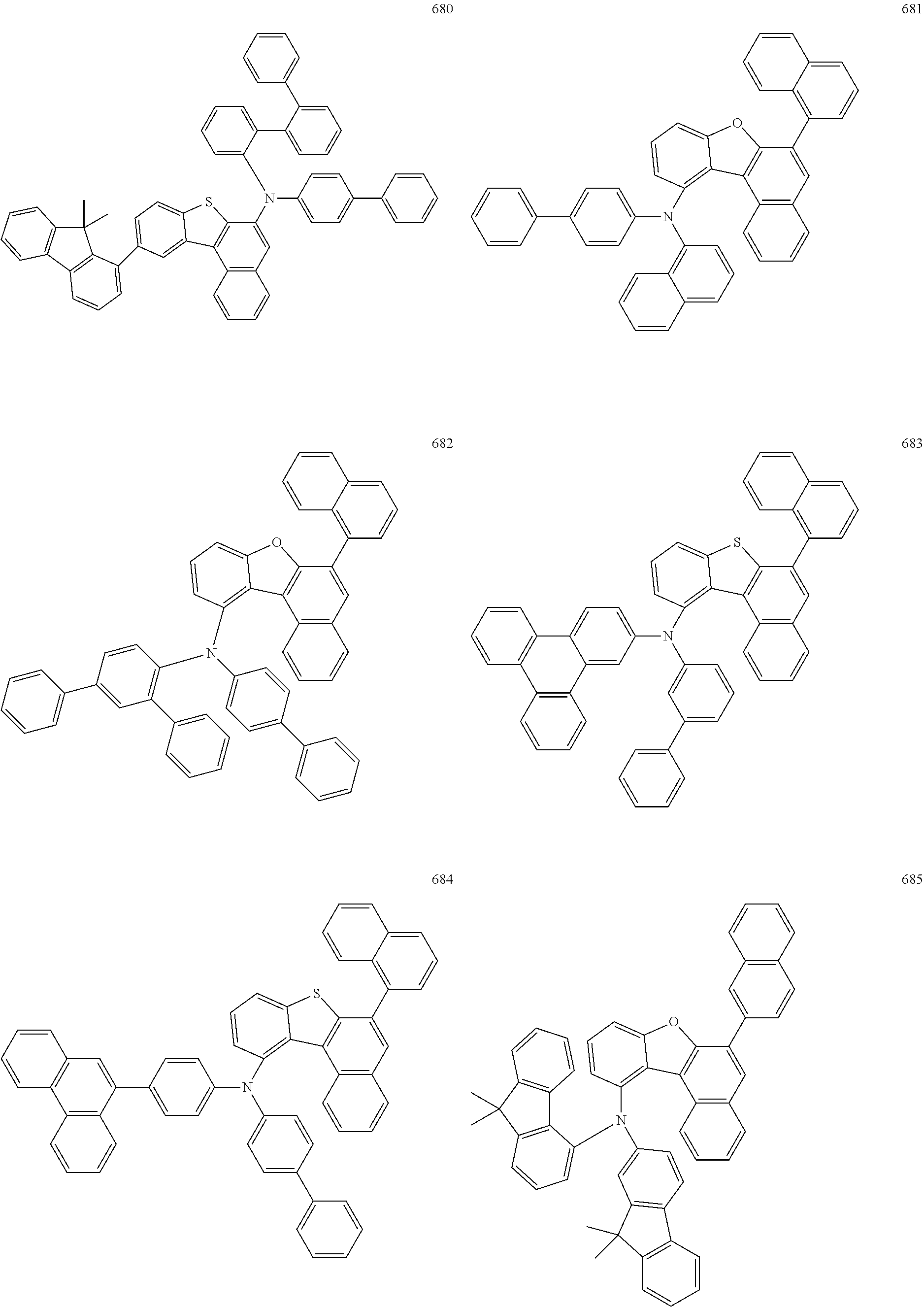

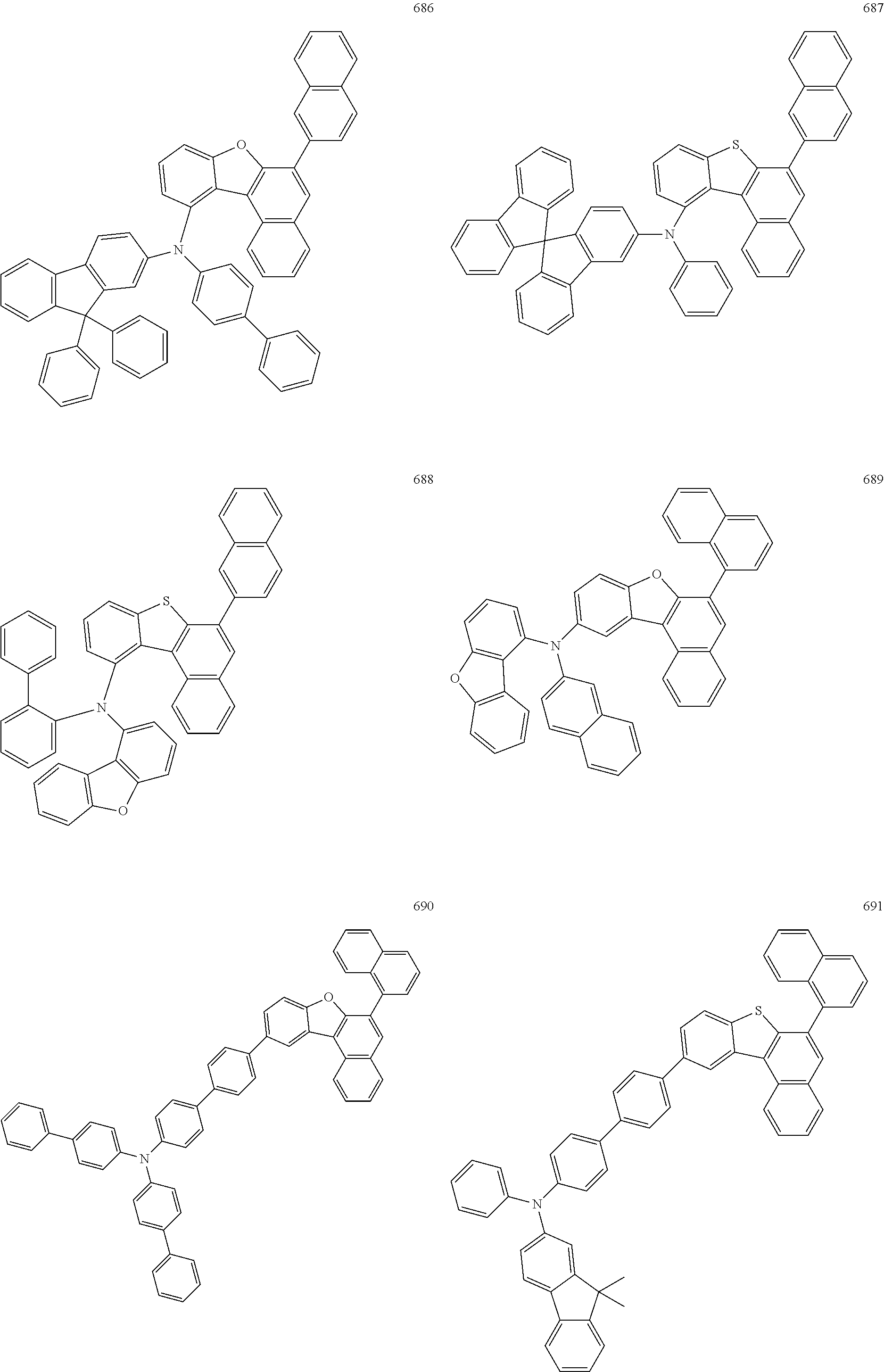

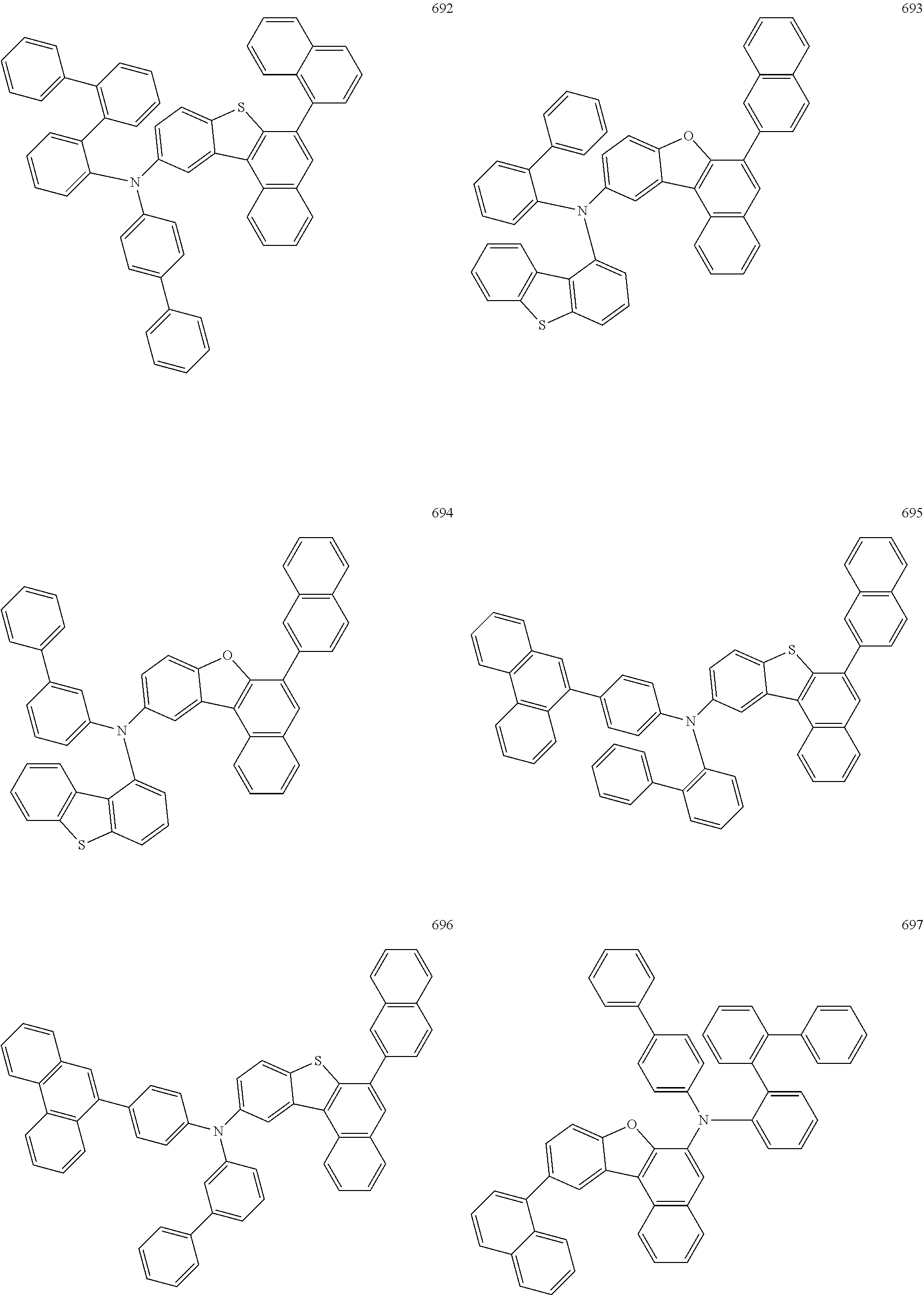

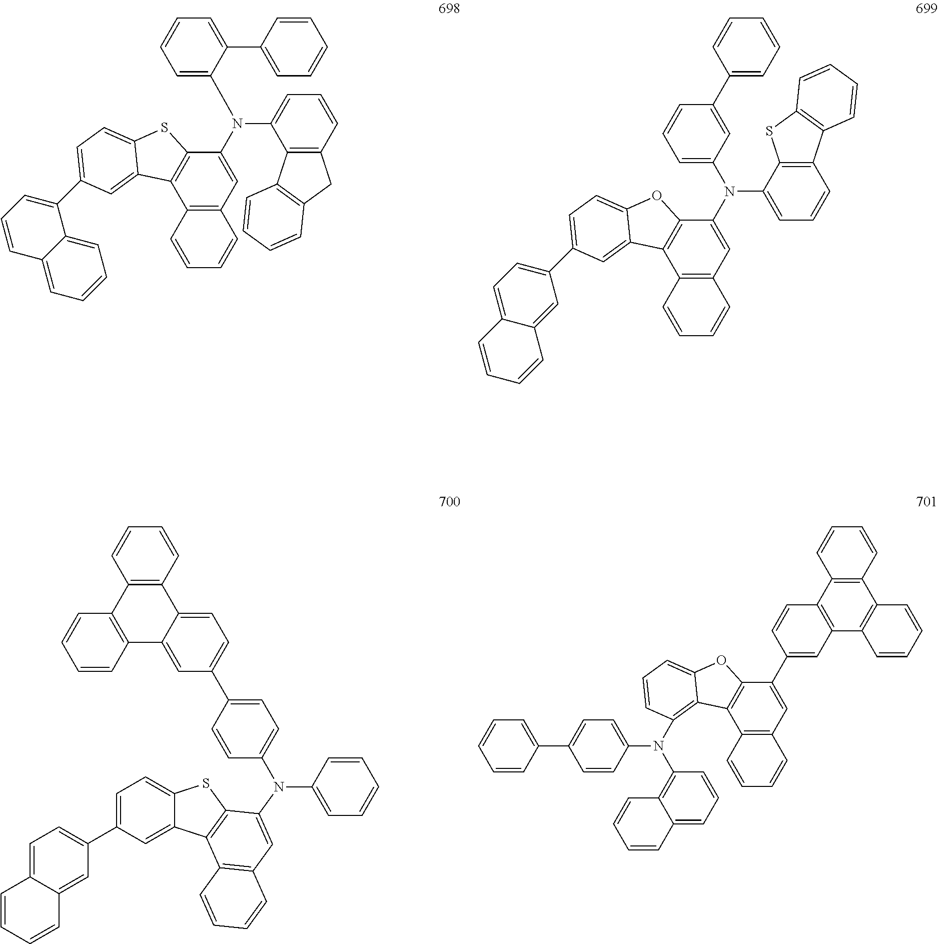 [SMILES: c1ccc(-c2ccccc2N(c2cccc3c2-c2ccccc2C3)c2cc3ccccc3c3c2sc2ccc(-c4cccc5ccccc45)cc23)cc1 ; c1ccc(-c2cccc(N(c3cc4ccccc4c4c3oc3ccc(-c5ccc6ccccc6c5)cc34)c3cccc4c3sc3ccccc34)c2)cc1 ; c1ccc(N(c2ccc(-c3ccc4c5ccccc5c5ccccc5c4c3)cc2)c2cc3ccccc3c3c2sc2ccc(-c4ccc5ccccc5c4)cc23)cc1 ; c1ccc(-c2ccc(N(c3cccc4ccccc34)c3cccc4oc5c(-c6ccc7c8ccccc8c8ccccc8c7c6)cc6ccccc6c5c34)cc2)cc1]

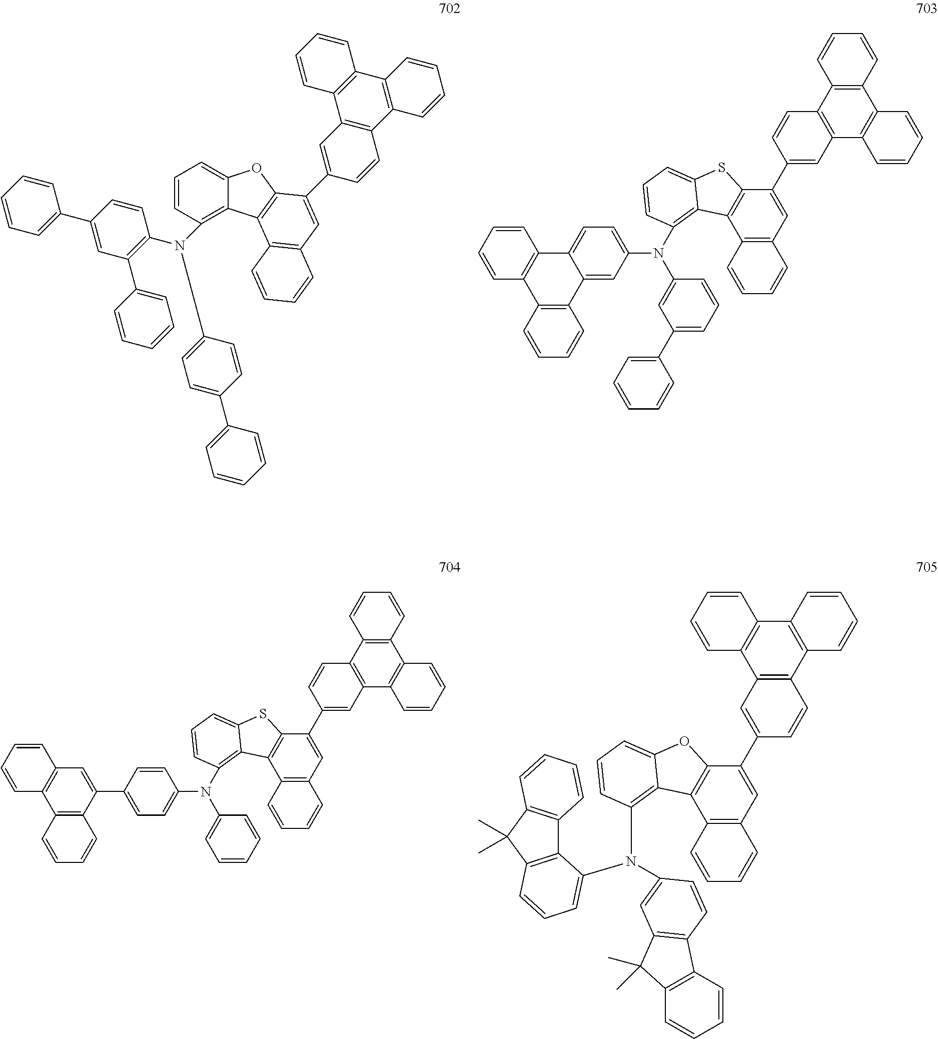 [SMILES: c1ccc(-c2ccc(N(c3ccc(-c4ccccc4)cc3-c3ccccc3)c3cccc4oc5c(-c6ccc7c8ccccc8c8ccccc8c7c6)cc6ccccc6c5c34)cc2)cc1 ; c1ccc(-c2cccc(N(c3ccc4c5ccccc5c5ccccc5c4c3)c3cccc4sc5c(-c6ccc7c8ccccc8c8ccccc8c7c6)cc6ccccc6c5c34)c2)cc1 ; c1ccc(N(c2ccc(-c3cc4ccccc4c4ccccc34)cc2)c2cccc3sc4c(-c5ccc6c7ccccc7c7ccccc7c6c5)cc5ccccc5c4c23)cc1 ; CC1(C)c2ccccc2-c2ccc(N(c3cccc4c3-c3ccccc3C4(C)C)c3cccc4oc5c(-c6ccc7c8ccccc8c8ccccc8c7c6)cc6ccccc6c5c34)cc21]

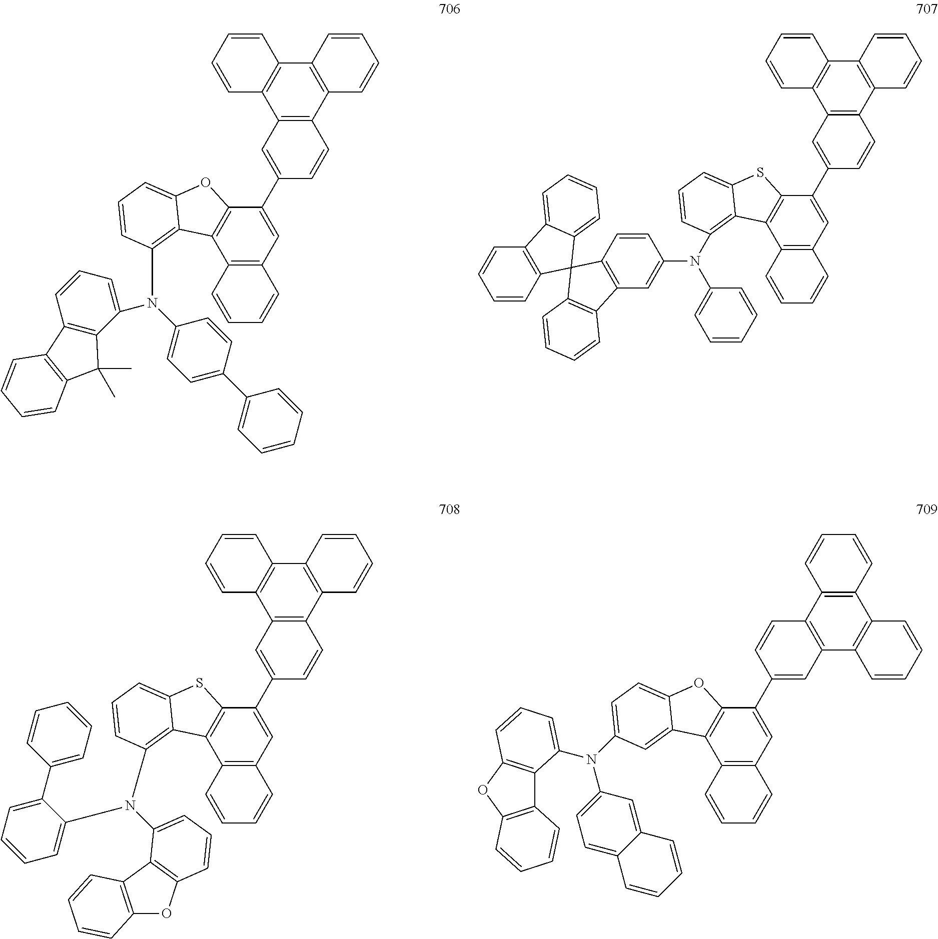 [SMILES: CC1(C)c2ccccc2-c2cccc(N(c3ccc(-c4ccccc4)cc3)c3cccc4oc5c(-c6ccc7c8ccccc8c8ccccc8c7c6)cc6ccccc6c5c34)c21 ; c1ccc(N(c2ccc3c(c2)-c2ccccc2C32c3ccccc3-c3ccccc32)c2cccc3sc4c(-c5ccc6c7ccccc7c7ccccc7c6c5)cc5ccccc5c4c23)cc1 ; c1ccc(-c2ccccc2N(c2cccc3oc4ccccc4c23)c2cccc3sc4c(-c5ccc6c7ccccc7c7ccccc7c6c5)cc5ccccc5c4c23)cc1 ; c1ccc2cc(N(c3ccc4oc5c(-c6ccc7c8ccccc8c8ccccc8c7c6)cc6ccccc6c5c4c3)c3cccc4oc5ccccc5c34)ccc2c1]

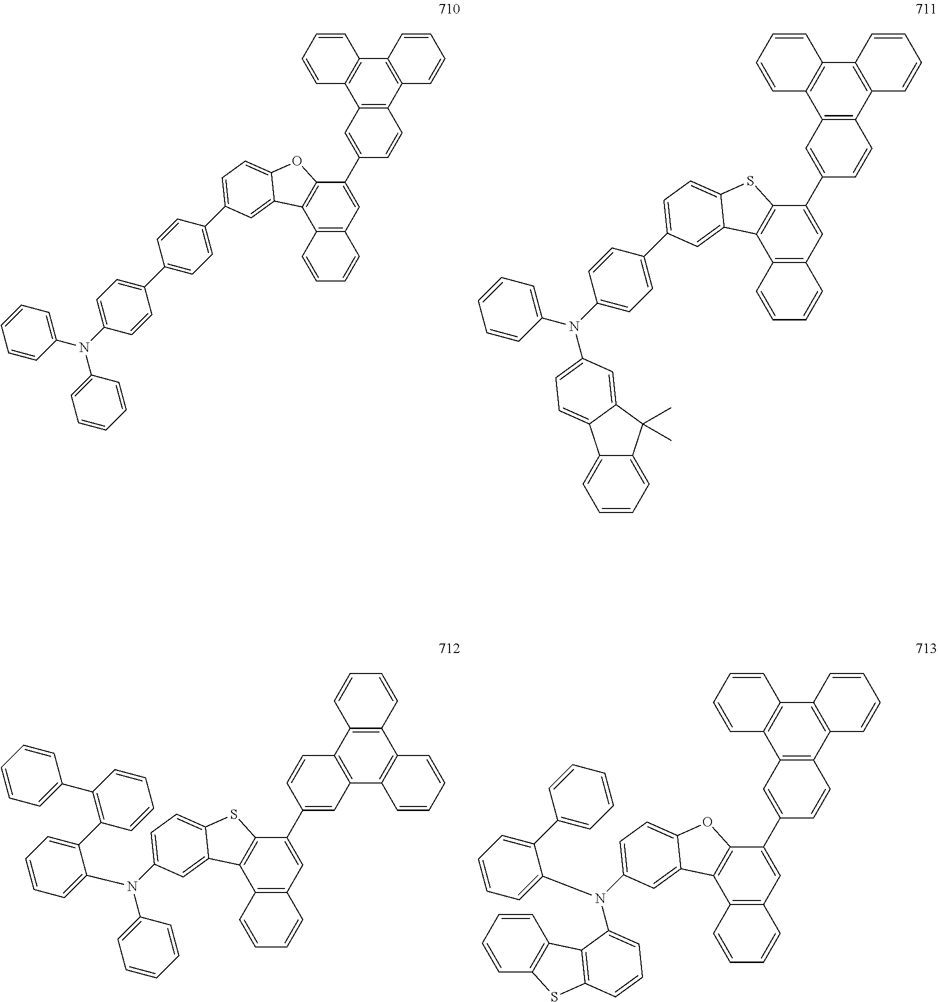 [SMILES: c1ccc(N(c2ccccc2)c2ccc(-c3ccc(-c4ccc5oc6c(-c7ccc8c9ccccc9c9ccccc9c8c7)cc7ccccc7c6c5c4)cc3)cc2)cc1 ; CC1(C)c2ccccc2-c2ccc(N(c3ccccc3)c3ccc(-c4ccc5sc6c(-c7ccc8c9ccccc9c9ccccc9c8c7)cc7ccccc7c6c5c4)cc3)cc21 ; c1ccc(-c2ccccc2-c2ccccc2N(c2ccccc2)c2ccc3sc4c(-c5ccc6c7ccccc7c7ccccc7c6c5)cc5ccccc5c4c3c2)cc1 ; c1ccc(-c2ccccc2N(c2ccc3oc4c(-c5ccc6c7ccccc7c7ccccc7c6c5)cc5ccccc5c4c3c2)c2cccc3sc4ccccc4c23)cc1]

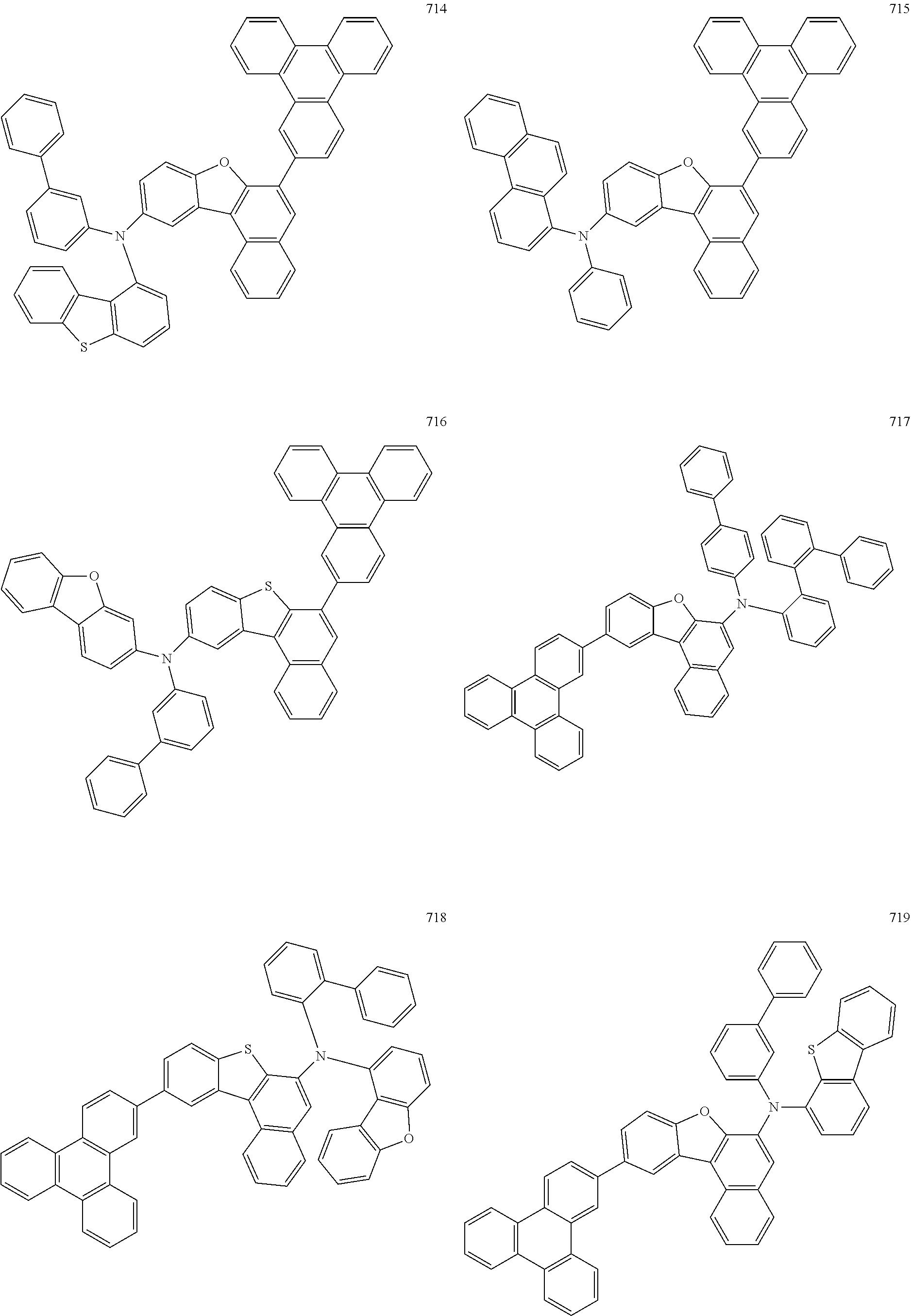

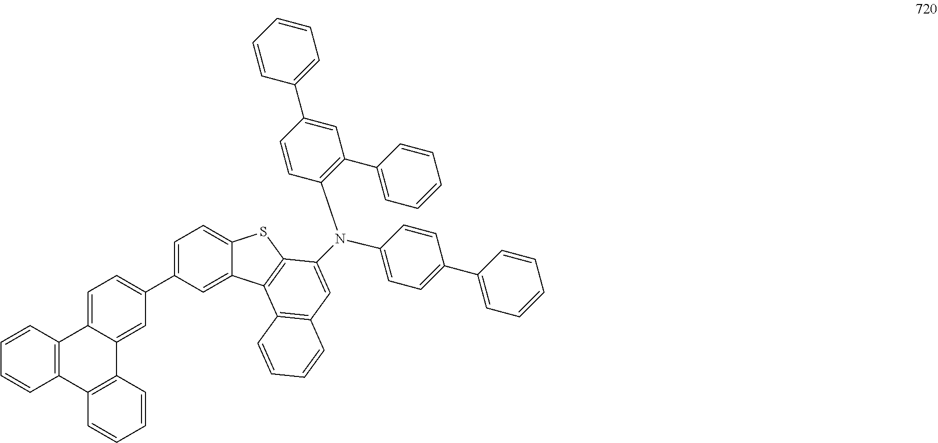 [SMILES: c1ccc(-c2ccc(N(c3ccc(-c4ccccc4)cc3-c3ccccc3)c3cc4ccccc4c4c3sc3ccc(-c5ccc6c7ccccc7c7ccccc7c6c5)cc34)cc2)cc1]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

Meanwhile, the compound has a high glass transition temperature (Tg) and thus has excellent thermal stability. The increase in thermal stability becomes an important factor for providing a device with driving stability.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include one or more of the heterocyclic compound represented by Chemical Formula 1.

In addition, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include one or more and three or less of the heterocyclic compound represented by Chemical Formula 1.

Furthermore, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include one or more and two or less of the heterocyclic compound represented by Chemical Formula 1.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocycle according to Chemical Formula 1 may be used as a material for the blue organic light emitting device.

In an exemplary embodiment of the present application, the organic light emitting device may be a green organic light emitting device, and the compound represented by Chemical Formula 1 may be used as a material for the green organic light emitting device.

In an exemplary embodiment of the present application, the organic light emitting device may be a red organic light emitting device, and the compound represented by Chemical Formula 1 may be used as a material for the red organic light emitting device.

In an exemplary embodiment of the present application, the organic light emitting device may be a blue organic light emitting device, and the heterocycle according to Chemical Formula 1 may be used as a material for a light emitting layer of the blue organic light emitting device.

In an exemplary embodiment of the present application, the organic light emitting device may be a green organic light emitting device, and the compound represented by Chemical Formula 1 may be used as a material for a light emitting layer of the green organic light emitting device.

In an exemplary embodiment of the present application, the organic light emitting device may be a red organic light emitting device, and the compound represented by Chemical Formula 1 may be used as a material for a light emitting layer of the red organic light emitting device.

In an exemplary embodiment of the present application, the organic material layer includes one or two of the heterocyclic compound represented by Chemical Formula 1, and may be used together with a phosphorescent dopant.

In an exemplary embodiment of the present application, the organic material layer includes one or two of the heterocyclic compound represented by Chemical Formula 1, and may be used together with an iridium-based dopant.

As a material for the phosphorescent dopant, those known in the art may be used.

For example, phosphorescent dopant materials represented by LL′MX′, LL′L″M, LMX′X″, L2MX′ and L3M may be used, but the scope of the present invention is not limited by these examples.

Here, L, L′, L″, X′ and X″ are bidentate ligands different from each other, and M is a metal forming an octahedral complex.

M may be iridium, platinum, osmium, and the like.

L, L′, and L″ are anionic, bidentate ligands coordinated on M by the iridium-based dopant by sp2 carbon and a heteroatom, and X may perform a function of trapping electrons or holes. Non-limiting examples of L, L′, and L″ include 2-(1-naphthyl)benzoxazole, (2-phenylbenzoxazole), (2-phenylbenzothiazole), (2-phenylbenzothiazole), (7,8-benzoquinoline), (thiophene group pyrizine), phenylpyridine, benzothiophene group pyrizine, 3-methoxy-2-phenylpyridine, thiophene group pyrizine, tolylpyridine, and the like. Non-limiting examples of X′ and X″ include acetylacetonate (acac), hexafluoroacetylacetonate, salicylidene, picolinate, 8-hydroxyquinolinate, and the like.

More specific examples thereof will be shown below, but the present application is not limited only to these examples.

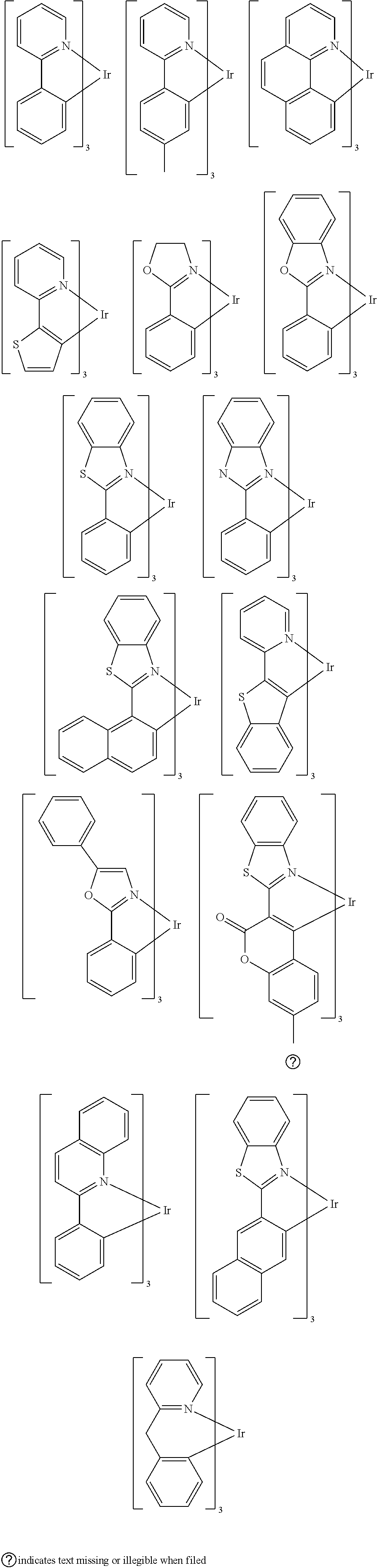

In an exemplary embodiment of the present application, as the iridium-based dopant, Ir(ppy)₃ as a green phosphorescent dopant may be used.

In an exemplary embodiment of the present application, as the iridium-based dopant, Ir(piq)₂(acac) may be used as a red phosphorescent dopant.

In an exemplary embodiment of the present application, the content of the dopant may be 1% to 15%, preferably 3% to 10%, and more preferably 5% to 10% based on the entire light emitting layer.

The specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, as the iridium-based dopant, Ir(ppy)₃ as a green phosphorescent dopant may be used.

In an exemplary embodiment of the present application, provided is an organic light emitting device, in which the organic material layer of the organic light emitting device includes a light emitting layer, and the light emitting layer includes the heterocyclic compound.

In an exemplary embodiment of the present application, provided is an organic light emitting device, in which the organic material layer of the organic light emitting device includes a light emitting layer, and the light emitting layer includes a host material, and the host material includes the heterocyclic compound.

In the organic light emitting device of the present invention, the organic material layer includes an electron injection layer or an electron transport layer, and the electron injection layer or electron transport layer may include the heterocyclic compound.

In another organic light emitting device, the organic material layer includes an electron blocking layer or a hole blocking layer, and the electron blocking layer or the hole blocking layer may include the heterocyclic compound.

In still another organic light emitting device, the organic material layer includes an electron transport layer, a light emitting layer or a hole blocking layer, and the electron transport layer, the light emitting layer or the hole blocking layer may include the heterocyclic compound.

The organic light emitting device of the present invention may further include one or two or more layers selected from the group consisting of a light emitting layer, a hole injection layer, a hole transport layer, an electron injection layer, an electron transport layer, an electron blocking layer, and a hole blocking layer.

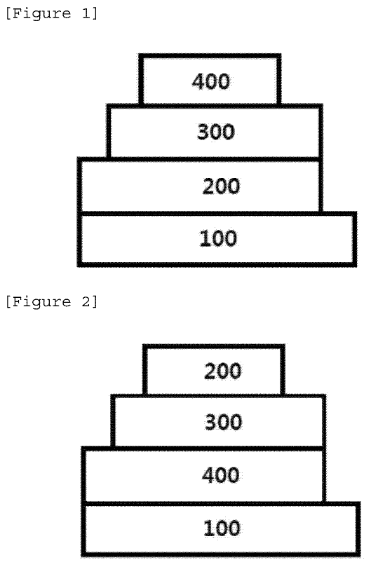

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented.

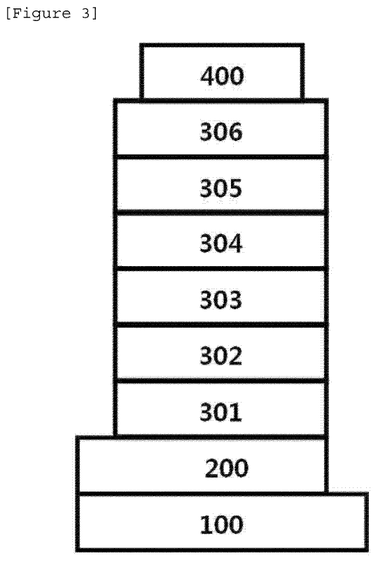

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

The organic light emitting device according to an exemplary embodiment of the present application may be manufactured by typical methods and materials for manufacturing an organic light emitting device, except that the organic material layer having one or more layers is formed by using one or two or more of the above-described heterocyclic compound represented by Chemical Formula 1.

In an exemplary embodiment of the present application, provided is a composition for an organic material layer, including the heterocyclic compound represented by Chemical Formula 1.

A material that may be included in the art in addition to the heterocyclic compound represented by Chemical Formula 1 may be included in the composition for an organic material layer.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by pre-mixing two of the heterocyclic compound represented by Chemical Formula 1, and using a thermal vacuum deposition method.

The pre-mixing means that before the compound of Chemical Formula 1 (N-type heterocyclic compound) and the compound of Chemical Formula 1 (P-type heterocyclic compound) are deposited onto an organic material layer, the materials are first mixed and the mixture is contained in one common container and mixed.

The pre-mixed material may be referred to as a composition for an organic material layer according to an exemplary embodiment of the present application.

In the organic light emitting device according to an exemplary embodiment of the present application, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

### MODE FOR INVENTION

Hereinafter, the present specification will be described in more detail through Examples, but these Examples are provided only for exemplifying the present application, and are not intended to limit the scope of the present application.

### PREPARATION EXAMPLES

### [Preparation Example 1] Preparation of Compound 001

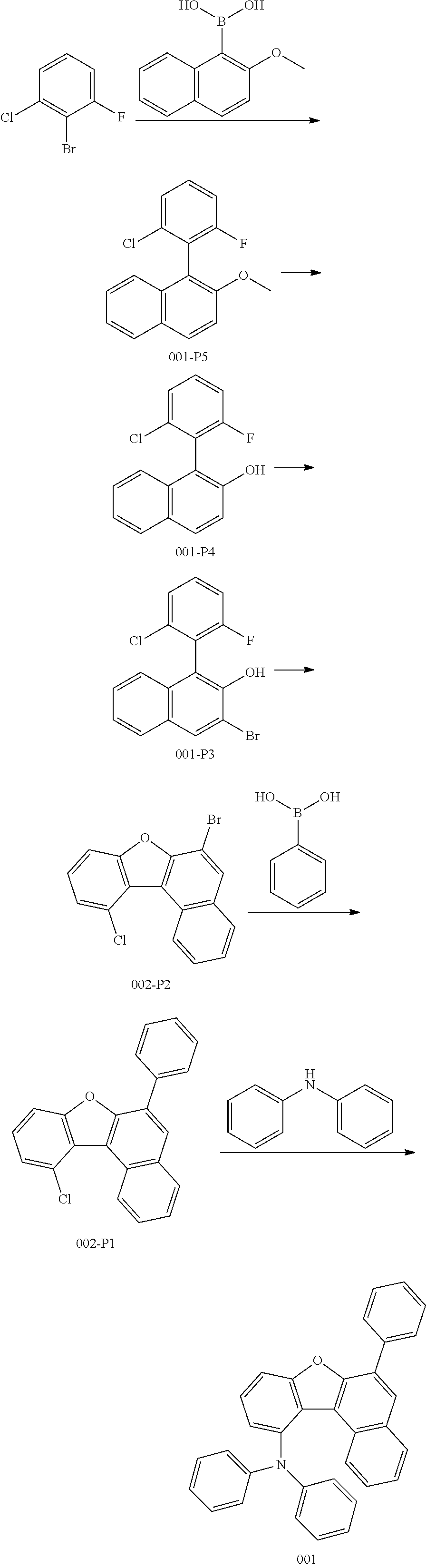

### 1) Preparation of Compound 001-P5

After a compound 2-bromo-1-chloro-3-fluorobenzene (30 g, 0.1432 mol) and (2-methoxynaphthalen-1-yl)boronic acid (31.8 g, 0.1576 mol) were dissolved in 500 ml of toluene, 100 mL of ethanol, and 150 mL of distilled water, Pd(PPh₃)₄ (8.3 g, 0.0072 mol) and K₂CO₃ (49.5 g, 0.3581 mol) were added thereto, and the resulting mixture was stirred under reflux for 12 hours. After the reaction was completed, extraction was performed using dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 001-P5 (30 g, 73%).

### 2) Preparation of Compound 001-P4

After Compound 001-P5 (30 g, 0.1046 mol) was dissolved in 500 mL of dichloromethane, BBr₃ (65.5 g, 0.2616 mol) was slowly added dropwise thereto, and then the resulting mixture was stirred under reflux at room temperature for 3 hours. After the reaction was completed, distilled water was slowly added thereto, extraction was performed using dichloromethane, the organic layer dried over anhydrous MgSO₄, and then the solvent was removed by a rotary evaporator, and then Compound 001-P4 (25 g, 88%) was obtained.

### 3) Preparation of Compound 001-P3

After Compound 001-P4 (25 g, 0.0917 mol) was dissolved in 350 mL of dimethylformamide, N-bromosuccinimide (18 g, 0.1008 mol) was added thereto, and the resulting mixture was stirred at room temperature for 8 hours. After the reaction was completed, extraction was performed using dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 001-P3 (28 g, 87%).

### 4) Preparation of Compound 001-P2

Compound 001-P3 (28 g, 0.0796 mol) and cesium carbonate (51.9 g, 0.1593 mol) were added to 400 mL of DMA, and the resulting mixture was stirred under reflux for 6 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 001-P2 (23 g, 87%).

### 5) Preparation of Compound 001-P1

After Compound 001-P2 (23 g, 0.0694 mol) and phenylboronic acid (10.2 g, 0.0832 mol) were dissolved in 200 ml of toluene, 50 ml of ethanol, and 100 ml of distilled water, Pd(PPh₃)₄ (4 g, 0.0035 mol) and K₂CO₃ (24 g, 0.1734 mol) were added thereto, and the resulting mixture was stirred under reflux for 12 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 001-P1 (16 g, 70%).

### 6) Preparation of Compound 001

After Compound 001-P1 (16 g, 0.0487 mol) and diphenylamine (9.1 g, 0.0535 mmol) were dissolved in 200 ml of toluene, Pd₂(dba)₃ (2.2 g, 0.0024 mol), xphos (3.5 g, 0.0073 mol), and t-BuONa (9.4 g, 0.0973 mol) were added thereto, and the resulting mixture was stirred under reflux for 3 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichioromethane and hexane as an eluting solvent, thereby obtaining Compound 001 (15 g, 67%) A target compound was synthesized by preparation in the same manner as in Preparation Example 1, except that Compound A in the following Table 1, Compound B in the following Table 1, and Compound C in the following Table 1 were used instead of 2-bromo-1-chloro-3-fluorobenzene, phenylboronic acid, and diphenylamine, respectively, in Preparation Example 1.

TABLE 1

| Com- | | | | | |
|---|---|---|---|---|---|
| pound | Compound | | | | |
| No. | A | Compound B | Compound C | Target Compound | Yield |
| 3 | 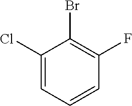 | 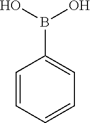 | 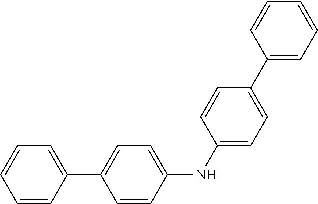 [SMILES: c1ccc(-c2ccc(Nc3ccc(-c4ccccc4)cc3)cc2)cc1] | 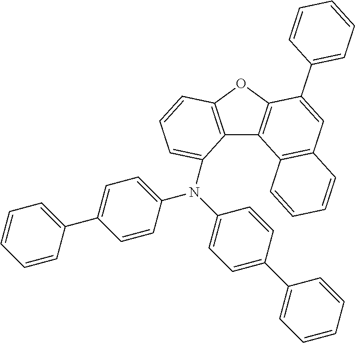 [SMILES: c1ccc(-c2ccc(N(c3ccc(-c4ccccc4)cc3)c3cccc4oc5c(-c6ccccc6)cc6ccccc6c5c34)cc2)cc1] | 69% |
| 5 | 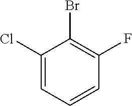 | 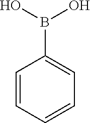 | 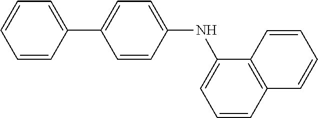 [SMILES: c1ccc(-c2ccc(Nc3cccc4ccccc34)cc2)cc1] | 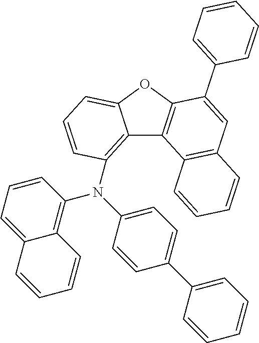 [SMILES: c1ccc(-c2ccc(N(c3cccc4ccccc34)c3cccc4oc5c(-c6ccccc6)cc6ccccc6c5c34)cc2)cc1] | 70% |
| 11 | 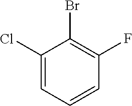 | 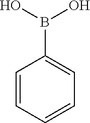 | 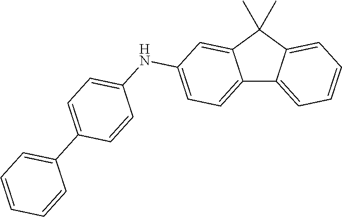 [SMILES: CC1(C)c2ccccc2-c2ccc(Nc3ccc(-c4ccccc4)cc3)cc21] | 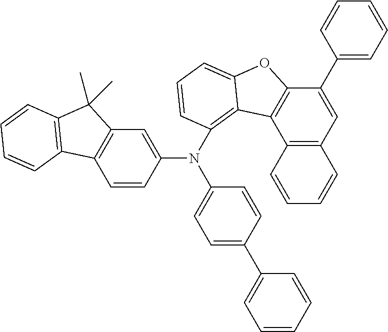 [SMILES: CC1(C)c2ccccc2-c2ccc(N(c3ccc(-c4ccccc4)cc3)c3cccc4oc5c(-c6ccccc6)cc6ccccc6c5c34)cc21] | 75% |
| 19 | 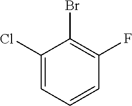 | 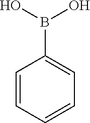 | 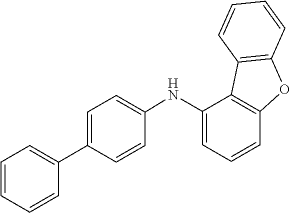 [SMILES: c1ccc(-c2ccc(Nc3cccc4oc5ccccc5c34)cc2)cc1] | 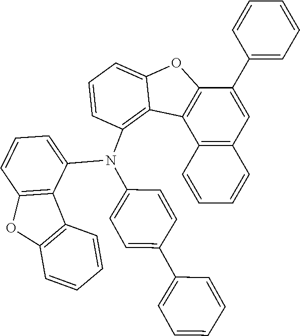 [SMILES: c1ccc(-c2ccc(N(c3cccc4oc5ccccc5c34)c3cccc4oc5c(-c6ccccc6)cc6ccccc6c5c34)cc2)cc1] | 65% |
| 29 | 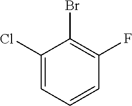 | 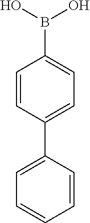 [SMILES: OB(O)c1ccc(-c2ccccc2)cc1] | 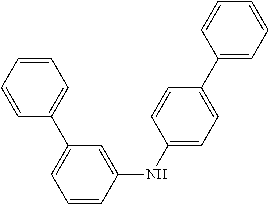 [SMILES: c1ccc(-c2ccc(Nc3cccc(-c4ccccc4)c3)cc2)cc1] | 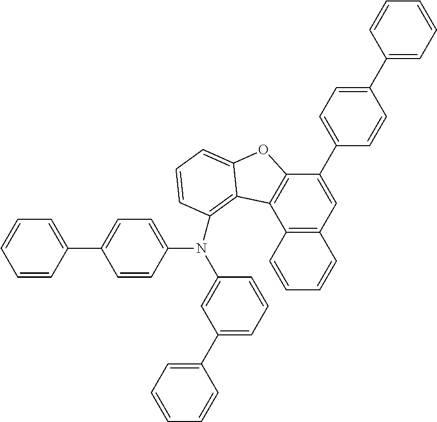 [SMILES: c1ccc(-c2ccc(-c3cc4ccccc4c4c3oc3cccc(N(c5ccc(-c6ccccc6)cc5)c5cccc(-c6ccccc6)c5)c34)cc2)cc1] | 66% |
| 56 | 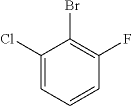 | 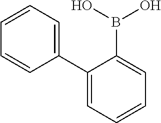 [SMILES: OB(O)c1ccccc1-c1ccccc1] | 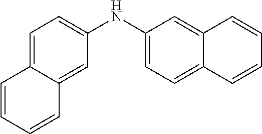 [SMILES: c1ccc2cc(Nc3ccc4ccccc4c3)ccc2c1] | 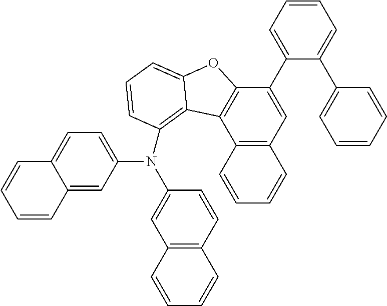 [SMILES: c1ccc(-c2ccccc2-c2cc3ccccc3c3c2oc2cccc(N(c4ccc5ccccc5c4)c4ccc5ccccc5c4)c23)cc1] | 69% |
| 85 | 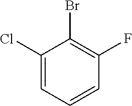 | 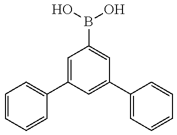 [SMILES: OB(O)c1cc(-c2ccccc2)cc(-c2ccccc2)c1] | 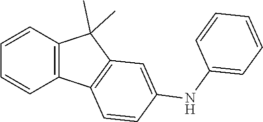 [SMILES: CC1(C)c2ccccc2-c2ccc(Nc3ccccc3)cc21] | 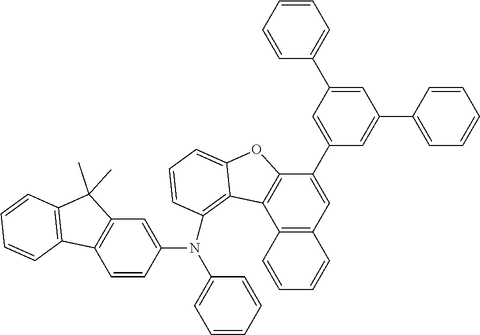 [SMILES: CC1(C)c2ccccc2-c2ccc(N(c3ccccc3)c3cccc4oc5c(-c6cc(-c7ccccc7)cc(-c7ccccc7)c6)cc6ccccc6c5c34)cc21] | 65% |
| 101 | 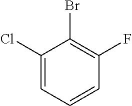 | 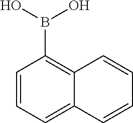 [SMILES: OB(O)c1cccc2ccccc12] | 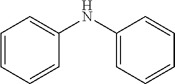 [SMILES: c1ccc(Nc2ccccc2)cc1] | 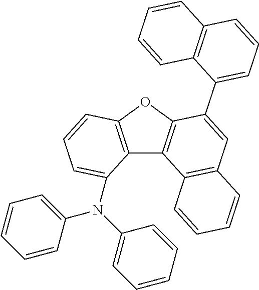 [SMILES: c1ccc(N(c2ccccc2)c2cccc3oc4c(-c5cccc6ccccc56)cc5ccccc5c4c23)cc1] | 66% |
| 107 | 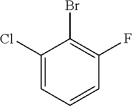 | 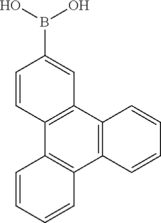 [SMILES: OB(O)c1ccc2c3ccccc3c3ccccc3c2c1] | 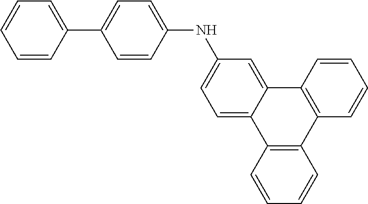 [SMILES: c1ccc(-c2ccc(Nc3ccc4c5ccccc5c5ccccc5c4c3)cc2)cc1] | 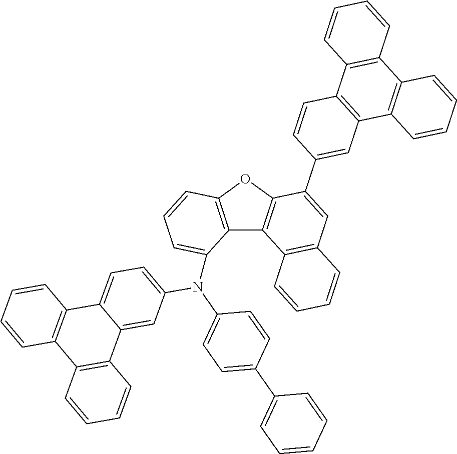 [SMILES: c1ccc(-c2ccc(N(c3ccc4c5ccccc5c5ccccc5c4c3)c3cccc4oc5c(-c6ccc7c8ccccc8c8ccccc8c7c6)cc6ccccc6c5c34)cc2)cc1] | 68% |
| 113 | 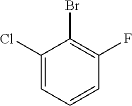 | 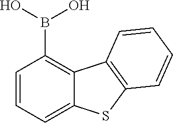 [SMILES: OB(O)c1cccc2sc3ccccc3c12] | 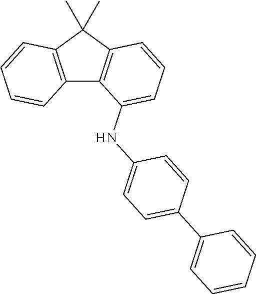 [SMILES: CC1(C)c2ccccc2-c2c(Nc3ccc(-c4ccccc4)cc3)cccc21] | 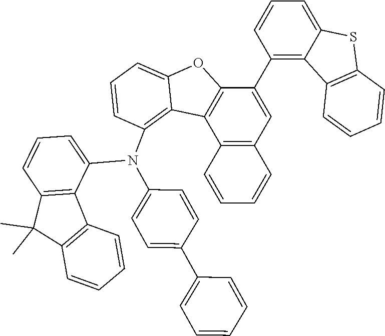 [SMILES: CC1(C)c2ccccc2-c2c(N(c3ccc(-c4ccccc4)cc3)c3cccc4oc5c(-c6cccc7sc8ccccc8c67)cc6ccccc6c5c34)cccc21] | 73% |
| 121 | 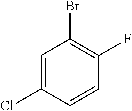 | 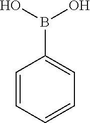 | 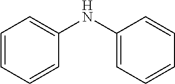 [SMILES: c1ccc(Nc2ccccc2)cc1] | 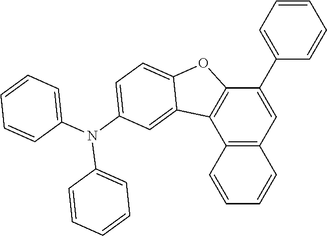 [SMILES: c1ccc(-c2cc3ccccc3c3c2oc2ccc(N(c4ccccc4)c4ccccc4)cc23)cc1] | 70% |
| 127 | 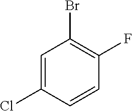 | 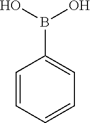 | 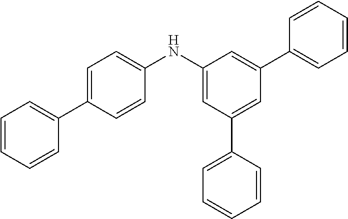 [SMILES: c1ccc(-c2ccc(Nc3cc(-c4ccccc4)cc(-c4ccccc4)c3)cc2)cc1] | 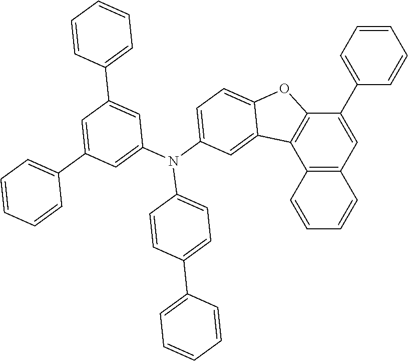 [SMILES: c1ccc(-c2ccc(N(c3cc(-c4ccccc4)cc(-c4ccccc4)c3)c3ccc4oc5c(-c6ccccc6)cc6ccccc6c5c4c3)cc2)cc1] | 72% |
| 128 | 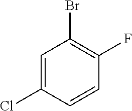 | 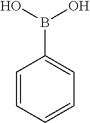 | 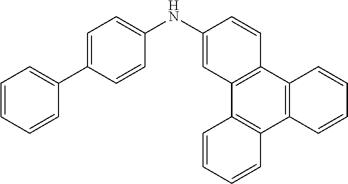 [SMILES: c1ccc(-c2ccc(Nc3ccc4c5ccccc5c5ccccc5c4c3)cc2)cc1] | 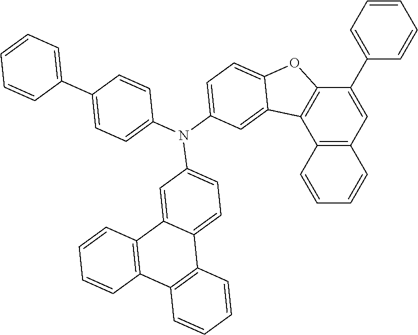 [SMILES: c1ccc(-c2ccc(N(c3ccc4c5ccccc5c5ccccc5c4c3)c3ccc4oc5c(-c6ccccc6)cc6ccccc6c5c4c3)cc2)cc1] | 71% |
| 129 | 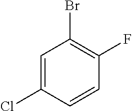 | 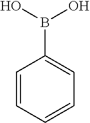 | 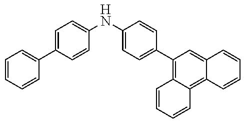 [SMILES: c1ccc(-c2ccc(Nc3ccc(-c4cc5ccccc5c5ccccc45)cc3)cc2)cc1] | 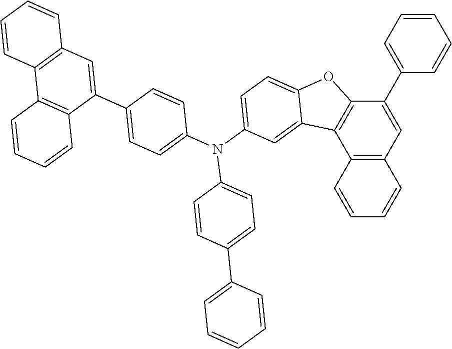 [SMILES: c1ccc(-c2ccc(N(c3ccc(-c4cc5ccccc5c5ccccc45)cc3)c3ccc4oc5c(-c6ccccc6)cc6ccccc6c5c4c3)cc2)cc1] | 73% |
| 130 | 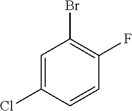 | 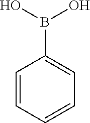 | 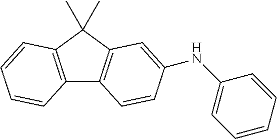 [SMILES: CC1(C)c2ccccc2-c2ccc(Nc3ccccc3)cc21] | 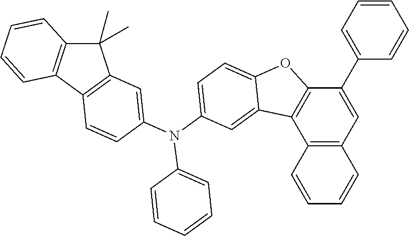 [SMILES: CC1(C)c2ccccc2-c2ccc(N(c3ccccc3)c3ccc4oc5c(-c6ccccc6)cc6ccccc6c5c4c3)cc21] | 75% |
| 133 | 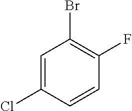 | 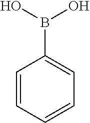 | 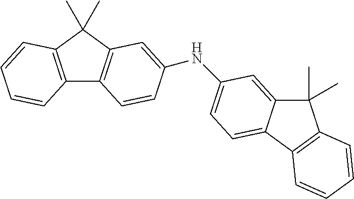 [SMILES: CC1(C)c2ccccc2-c2ccc(Nc3ccc4c(c3)C(C)(C)c3ccccc3-4)cc21] | 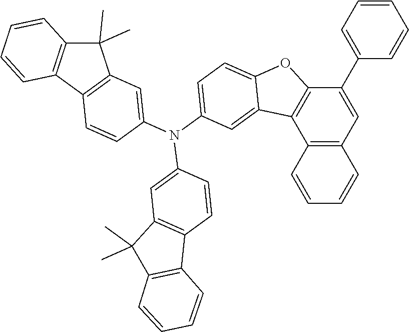 [SMILES: CC1(C)c2ccccc2-c2ccc(N(c3ccc4c(c3)C(C)(C)c3ccccc3-4)c3ccc4oc5c(-c6ccccc6)cc6ccccc6c5c4c3)cc21] | 68% |
| 136 | 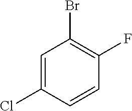 | 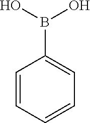 | 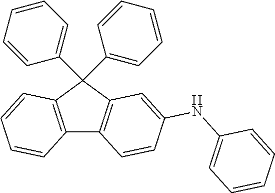 [SMILES: c1ccc(Nc2ccc3c(c2)C(c2ccccc2)(c2ccccc2)c2ccccc2-3)cc1] | 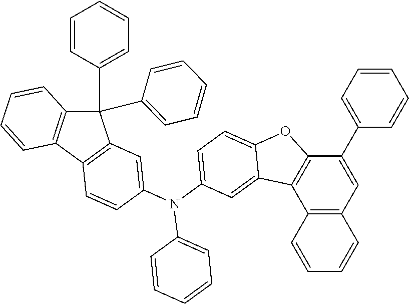 [SMILES: c1ccc(-c2cc3ccccc3c3c2oc2ccc(N(c4ccccc4)c4ccc5c(c4)C(c4ccccc4)(c4ccccc4)c4ccccc4-5)cc23)cc1] | 70% |
| 137 | 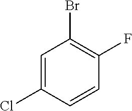 | 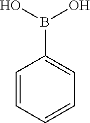 | 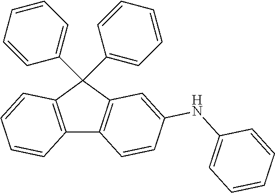 [SMILES: c1ccc(Nc2ccc3c(c2)C(c2ccccc2)(c2ccccc2)c2ccccc2-3)cc1] | 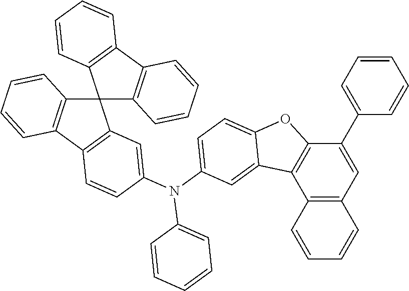 [SMILES: c1ccc(-c2cc3ccccc3c3c2oc2ccc(N(c4ccccc4)c4ccc5c(c4)C4(c6ccccc6-c6ccccc64)c4ccccc4-5)cc23)cc1] | 71% |
| 163 | 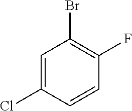 | 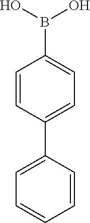 [SMILES: OB(O)c1ccc(-c2ccccc2)cc1] | 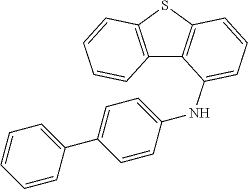 [SMILES: c1ccc(-c2ccc(Nc3cccc4sc5ccccc5c34)cc2)cc1] | 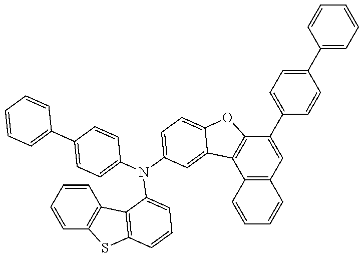 [SMILES: c1ccc(-c2ccc(-c3cc4ccccc4c4c3oc3ccc(N(c5ccc(-c6ccccc6)cc5)c5cccc6sc7ccccc7c56)cc34)cc2)cc1] | 69% |
| 189 | 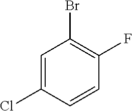 | 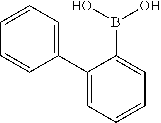 [SMILES: OB(O)c1ccccc1-c1ccccc1] | 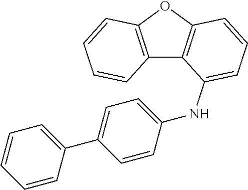 [SMILES: c1ccc(-c2ccc(Nc3cccc4oc5ccccc5c34)cc2)cc1] | 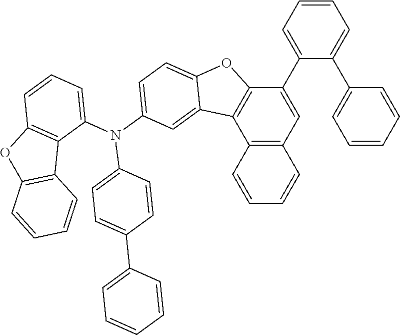 [SMILES: c1ccc(-c2ccc(N(c3ccc4oc5c(-c6ccccc6-c6ccccc6)cc6ccccc6c5c4c3)c3cccc4oc5ccccc5c34)cc2)cc1] | 71% |
| 232 | 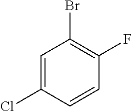 | 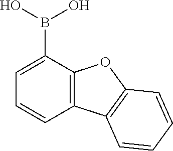 [SMILES: OB(O)c1cccc2c1oc1ccccc12] | 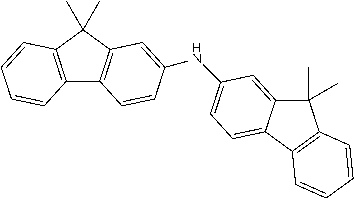 [SMILES: CC1(C)c2ccccc2-c2ccc(Nc3ccc4c(c3)C(C)(C)c3ccccc3-4)cc21] | 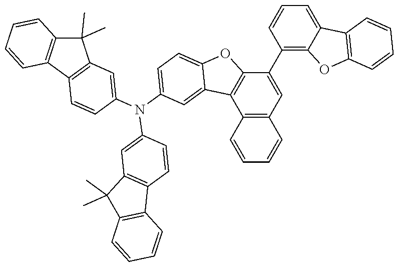 [SMILES: CC1(C)c2ccccc2-c2ccc(N(c3ccc4c(c3)C(C)(C)c3ccccc3-4)c3ccc4oc5c(-c6cccc7c6oc6ccccc67)cc6ccccc6c5c4c3)cc21] | 70% |
| 236 | 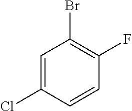 | 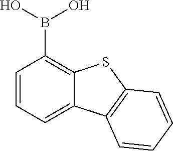 [SMILES: OB(O)c1cccc2c1sc1ccccc12] | 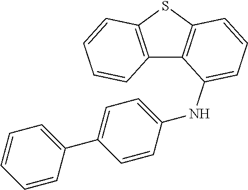 [SMILES: c1ccc(-c2ccc(Nc3cccc4sc5ccccc5c34)cc2)cc1] | 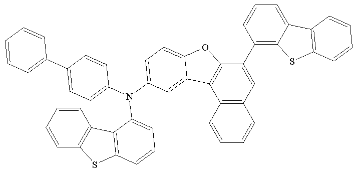 [SMILES: c1ccc(-c2ccc(N(c3ccc4oc5c(-c6cccc7c6sc6ccccc67)cc6ccccc6c5c4c3)c3cccc4sc5ccccc5c34)cc2)cc1] | 68% |

### [Preparation Example 2] Preparation of Compound 020

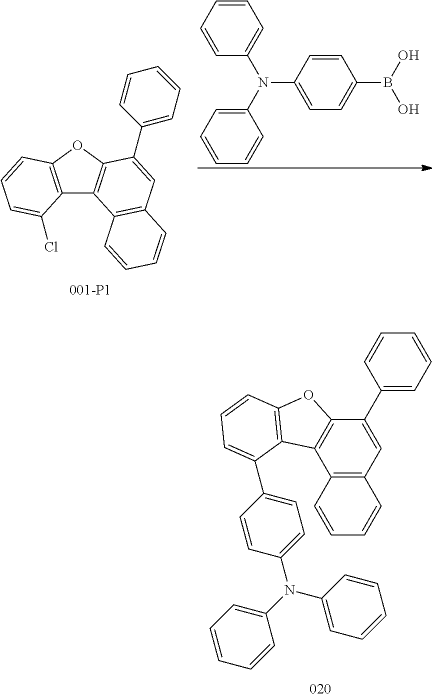

### Preparation of Compound 020

After Compound 001-P1 (16 g, 0.0487 mol) and (4-(diphenylamino)phenyl)boronic acid (16.9 g, 0.0584 mol) were dissolved in 250 mL of toluene and 50 mL of dissolved water, Pd₂(dba)₃ (2.23 g, 0.0024 mol), xphos (3.5 g, 0.0073 mol), and NaOH (3.9 g, 0.0973 mol) were added thereto, and the resulting mixture was stirred under reflux for 6 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 020 (22 g, 84%).

A target compound in the following Table 2 was synthesized by preparation in the same manner as in Preparation Example 2, except that Compound D and Compound E were used instead of 001-P1 and (4-(diphenylamino)phenyl)boronic acid, respectively, in

### Preparation Example 2

TABLE 2

| Com- | | | | |
|---|---|---|---|---|
| pound | | | Target | |
| No. | Compound D | Compound E | Compound | Yield |
| 70 | 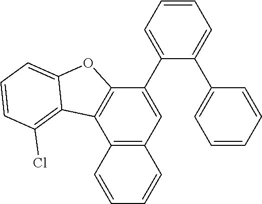 [SMILES: Clc1cccc2oc3c(-c4ccccc4-c4ccccc4)cc4ccccc4c3c12] | 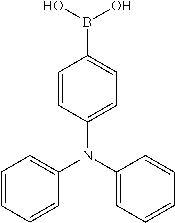 [SMILES: OB(O)c1ccc(N(c2ccccc2)c2ccccc2)cc1] | 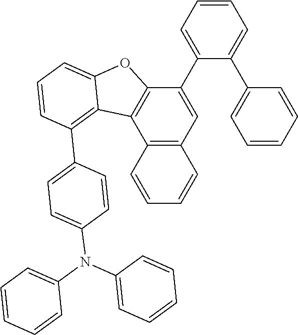 [SMILES: c1ccc(-c2ccccc2-c2cc3ccccc3c3c2oc2cccc(-c4ccc(N(c5ccccc5)c5ccccc5)cc4)c23)cc1] | 81% |
| 96 | 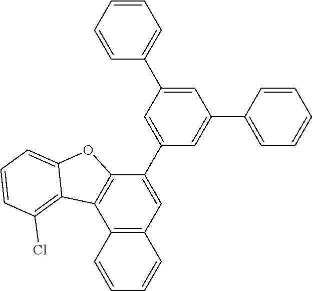 [SMILES: Clc1cccc2oc3c(-c4cc(-c5ccccc5)cc(-c5ccccc5)c4)cc4ccccc4c3c12] | 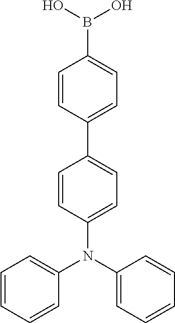 [SMILES: OB(O)c1ccc(-c2ccc(N(c3ccccc3)c3ccccc3)cc2)cc1] | 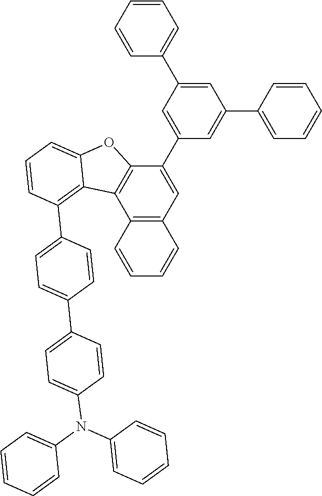 [SMILES: c1ccc(-c2cc(-c3ccccc3)cc(-c3cc4ccccc4c4c3oc3cccc(-c5ccc(-c6ccc(N(c7ccccc7)c7ccccc7)cc6)cc5)c34)c2)cc1] | 83% |
| 192 | 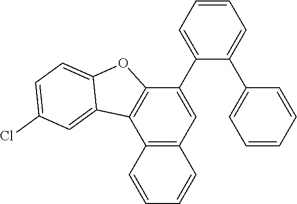 [SMILES: Clc1ccc2oc3c(-c4ccccc4-c4ccccc4)cc4ccccc4c3c2c1] | 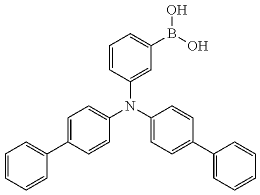 [SMILES: OB(O)c1cccc(N(c2ccc(-c3ccccc3)cc2)c2ccc(-c3ccccc3)cc2)c1] | 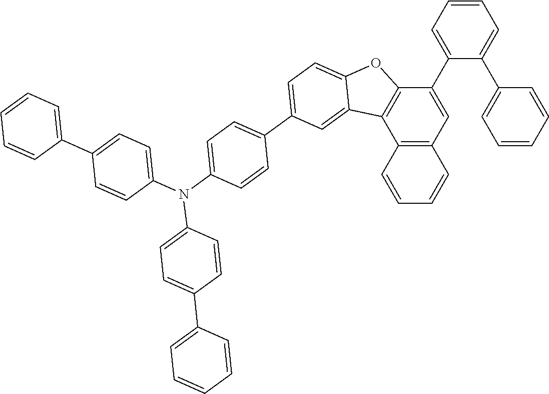 [SMILES: c1ccc(-c2ccc(N(c3ccc(-c4ccccc4)cc3)c3ccc(-c4ccc5oc6c(-c7ccccc7-c7ccccc7)cc7ccccc7c6c5c4)cc3)cc2)cc1] | 88% |
| 193 | 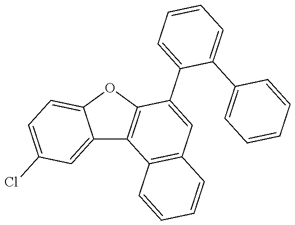 [SMILES: Clc1ccc2oc3c(-c4ccccc4-c4ccccc4)cc4ccccc4c3c2c1] | 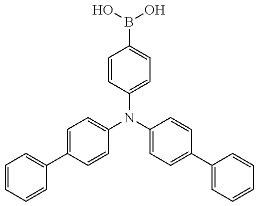 [SMILES: OB(O)c1ccc(N(c2ccc(-c3ccccc3)cc2)c2ccc(-c3ccccc3)cc2)cc1] | 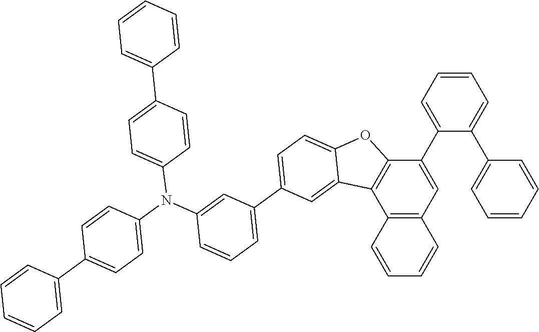 [SMILES: c1ccc(-c2ccc(N(c3ccc(-c4ccccc4)cc3)c3cccc(-c4ccc5oc6c(-c7ccccc7-c7ccccc7)cc7ccccc7c6c5c4)c3)cc2)cc1] | 80% |
| 238 | 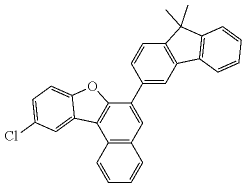 [SMILES: CC1(C)c2ccccc2-c2cc(-c3cc4ccccc4c4c3oc3ccc(Cl)cc34)ccc21] | 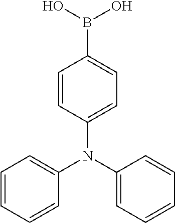 [SMILES: OB(O)c1ccc(N(c2ccccc2)c2ccccc2)cc1] | 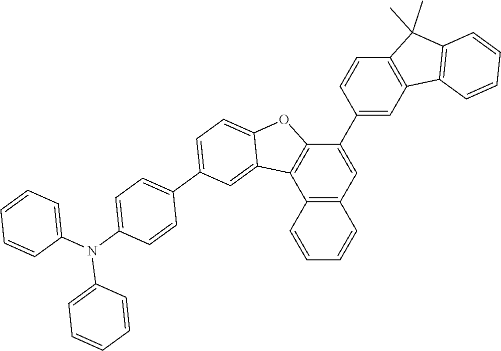 [SMILES: CC1(C)c2ccccc2-c2cc(-c3cc4ccccc4c4c3oc3ccc(-c5ccc(N(c6ccccc6)c6ccccc6)cc5)cc34)ccc21] | 81% |
| 240 | 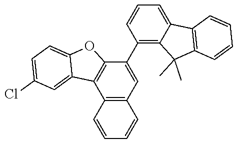 [SMILES: CC1(C)c2ccccc2-c2cccc(-c3cc4ccccc4c4c3oc3ccc(Cl)cc34)c21] | 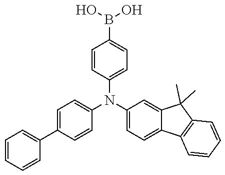 [SMILES: CC1(C)c2ccccc2-c2ccc(N(c3ccc(B(O)O)cc3)c3ccc(-c4ccccc4)cc3)cc21] | 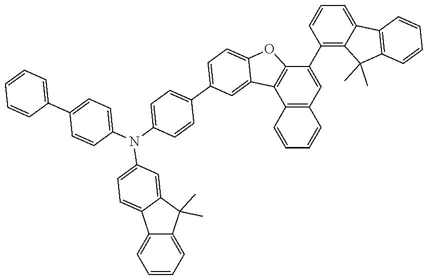 [SMILES: CC1(C)c2ccccc2-c2ccc(N(c3ccc(-c4ccccc4)cc3)c3ccc(-c4ccc5oc6c(-c7cccc8c7C(C)(C)c7ccccc7-8)cc7ccccc7c6c5c4)cc3)cc21] | 79% |

### [Preparation Example 3] Preparation of Compound 501

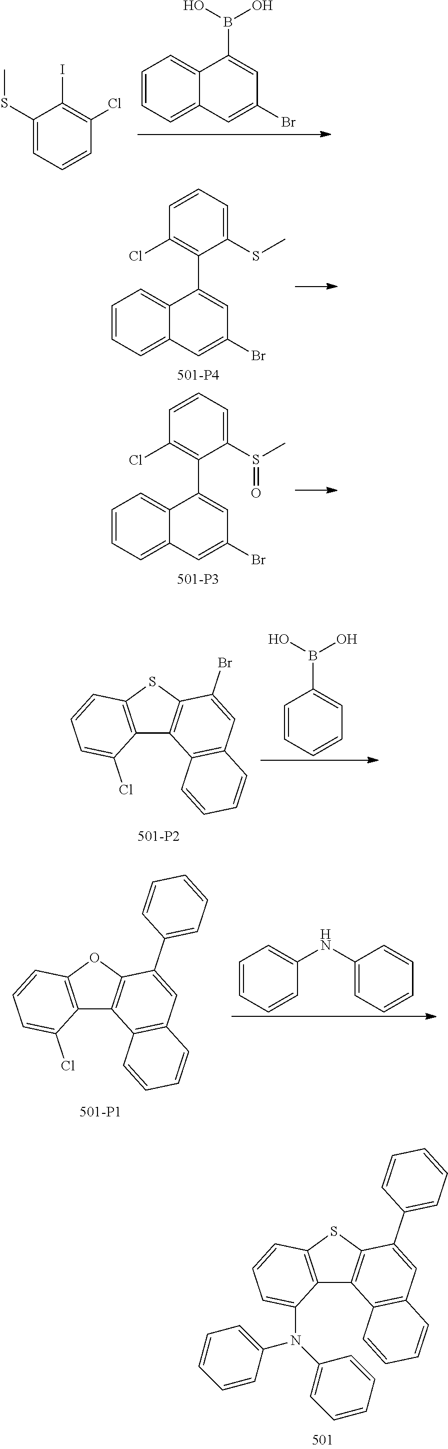

### 1) Preparation of Compound 501-P4

After a compound (3-chloro-2-iodophenyl) (methyl)sulfane (30 g, 0.1054 mol) and (3-bromonaphthalen-1-yl)boronic acid (29.1 g, 0.1160 mol) were dissolved in 500 ml of toluene, 100 mL of ethanol, and 100 mL of distilled water, Pd(PPh₃)₄ (6.1 g, 0.0053 mol) and K₂CO₃ (29.1 g, 0.2109 mol) were added thereto, and the resulting mixture was stirred under reflux for 8 hours. After the reaction was completed, extraction was performed using dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 501-P4 (31 g, 81%).

### 2) Preparation of Compound 501-P3

After Compound 501-P4 (31 g, 0.0852 mol) and hydrogen peroxide (10 mL) were dissolved in acetic acid (350 mL), the resulting solution was stirred at room temperature for 3 hours. After the reaction was completed, the acetic acid was removed, water was added thereto to obtain a solid, and then the solid was dissolved in dichloromethane, and the resulting solution was purified with column chromatography using dichloromethane and hexane as developing solvents to obtain Compound 501-P3 (28 g, 86%).

### 3) Preparation of Compound 501-P2

After Compound 501-P3 (28 g, 0.1001 mol) was dissolved in excess sulfuric acid (90 mL), the resulting solution was stirred at room temperature for 6 hours. After the reaction was completed, the reaction product was neutralized with an aqueous NaOH solution, and then extraction was performed using dichloromethane, the organic layer was dried over anhydrous MgSO₄, and then the solvent was removed by a rotary evaporator, and then the residue was purified by column chromatography using dichloromethane and hexane as an eluting solvent, thereby obtaining Compound 501-P2 (30 g, 86%).

### 4) Preparation of Compound 501-P1

After Compound 501-P2 (30 g, 0.0863 mol) and phenylboronic acid (12.63 g, 0.1036 mol) were dissolved in 400 mL of toluene, 100 mL of ethanol, and 100 mL of distilled water, Pd(PPh₃)₄ (5 g, 0.0043 mol) and K₂CO₃ (24 g, 0.1726 mol) were added thereto, and the resulting mixture was stirred under reflux for 8 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 501-P1 (22 g, 74%).

### 5) Preparation of Compound 501

After Compound 501-P1 (22 g, 0.0638 mol) and diphenylamine (11.9 g, 0.0702 mmol) were dissolved in 300 ml of toluene, Pd₂(dba)₃ (2.9 g, 0.0032 mol), xphos (4.6 g, 0.0096 mol), and t-BuONa (12.3 g, 0.1276 mol) were added thereto, and the resulting mixture was stirred under reflux for 3 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 501 (22 g, 72%).

A target compound in the following Table 3 was synthesized by preparation in the same manner as in Preparation Example 3, except that Compound F, Compound G, and Compound H were used instead of (3-chloro-2-iodophenyl) (methyl)sulfane, phenylboronic acid, and diphenylamine, respectively, in Preparation Example 3.

TABLE 3

| Com- | | | | | |
|---|---|---|---|---|---|
| pound | Compound | | | | |
| No. | F | Compound G | Compound H | Target Compound | Yield |
| 502 | 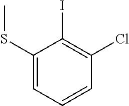 | 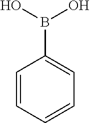 | 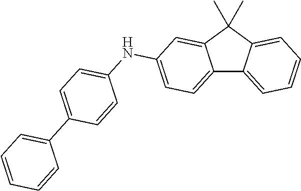 [SMILES: CC1(C)c2ccccc2-c2ccc(Nc3ccc(-c4ccccc4)cc3)cc21] | 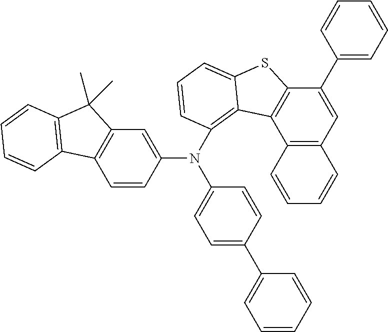 [SMILES: CC1(C)c2ccccc2-c2ccc(N(c3ccc(-c4ccccc4)cc3)c3cccc4sc5c(-c6ccccc6)cc6ccccc6c5c34)cc21] | 75% |
| 512 | 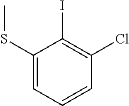 | 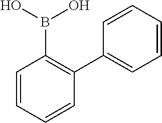 [SMILES: OB(O)c1ccccc1-c1ccccc1] | 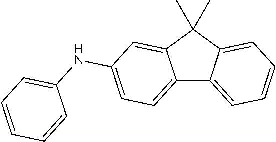 [SMILES: CC1(C)c2ccccc2-c2ccc(Nc3ccccc3)cc21] | 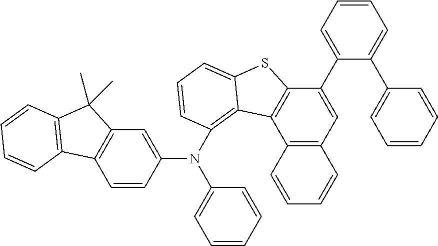 [SMILES: CC1(C)c2ccccc2-c2ccc(N(c3ccccc3)c3cccc4sc5c(-c6ccccc6-c6ccccc6)cc6ccccc6c5c34)cc21] | 72% |
| 522 | 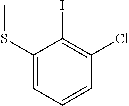 | 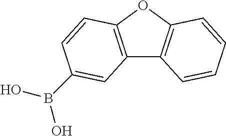 [SMILES: OB(O)c1ccc2oc3ccccc3c2c1] | 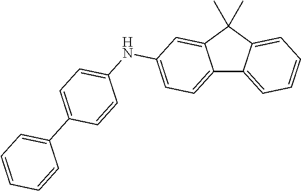 [SMILES: CC1(C)c2ccccc2-c2ccc(Nc3ccc(-c4ccccc4)cc3)cc21] | 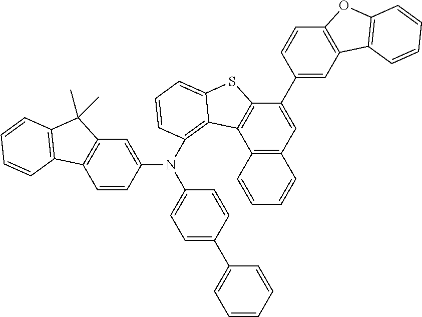 [SMILES: CC1(C)c2ccccc2-c2ccc(N(c3ccc(-c4ccccc4)cc3)c3cccc4sc5c(-c6ccc7oc8ccccc8c7c6)cc6ccccc6c5c34)cc21] | 77% |
| 525 | 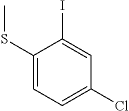 | 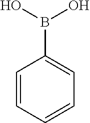 | 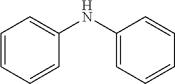 [SMILES: c1ccc(Nc2ccccc2)cc1] | 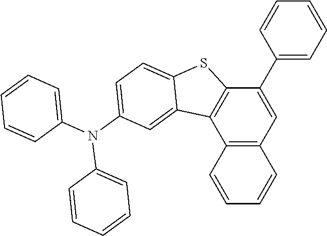 [SMILES: c1ccc(-c2cc3ccccc3c3c2sc2ccc(N(c4ccccc4)c4ccccc4)cc23)cc1] | 74% |
| 528 | 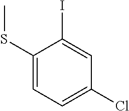 | 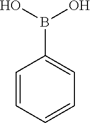 | 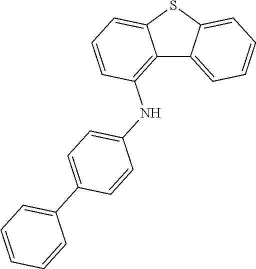 [SMILES: c1ccc(-c2ccc(Nc3cccc4sc5ccccc5c34)cc2)cc1] | 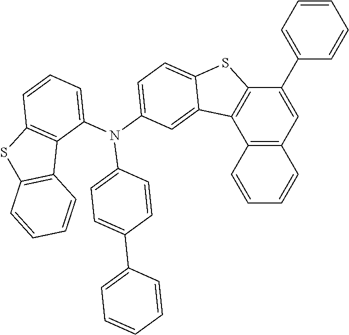 [SMILES: c1ccc(-c2ccc(N(c3ccc4sc5c(-c6ccccc6)cc6ccccc6c5c4c3)c3cccc4sc5ccccc5c34)cc2)cc1] | 69% |
| 539 | 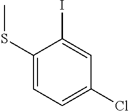 | 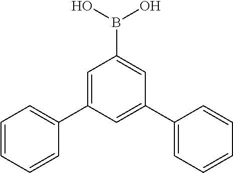 [SMILES: OB(O)c1cc(-c2ccccc2)cc(-c2ccccc2)c1] | 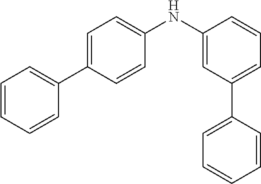 [SMILES: c1ccc(-c2ccc(Nc3cccc(-c4ccccc4)c3)cc2)cc1] | 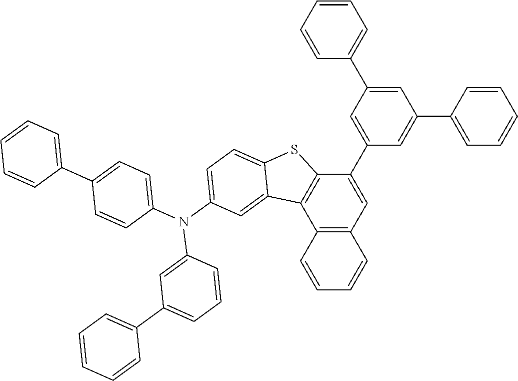 [SMILES: c1ccc(-c2ccc(N(c3cccc(-c4ccccc4)c3)c3ccc4sc5c(-c6cc(-c7ccccc7)cc(-c7ccccc7)c6)cc6ccccc6c5c4c3)cc2)cc1] | 71% |
| 548 | 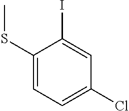 | 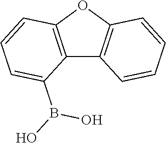 [SMILES: OB(O)c1cccc2oc3ccccc3c12] | 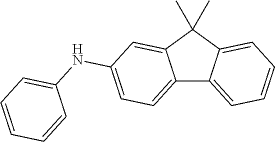 [SMILES: CC1(C)c2ccccc2-c2ccc(Nc3ccccc3)cc21] | 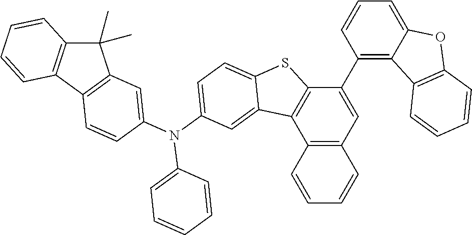 [SMILES: CC1(C)c2ccccc2-c2ccc(N(c3ccccc3)c3ccc4sc5c(-c6cccc7oc8ccccc8c67)cc6ccccc6c5c4c3)cc21] | 70% |
| 549 | 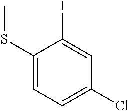 | 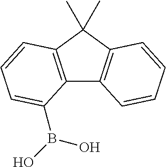 [SMILES: CC1(C)c2ccccc2-c2c(B(O)O)cccc21] | 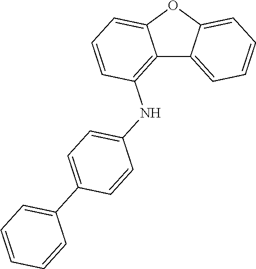 [SMILES: c1ccc(-c2ccc(Nc3cccc4oc5ccccc5c34)cc2)cc1] | 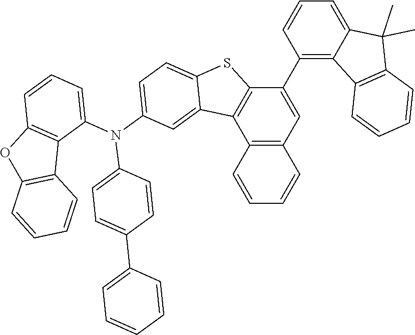 [SMILES: CC1(C)c2ccccc2-c2c(-c3cc4ccccc4c4c3sc3ccc(N(c5ccc(-c6ccccc6)cc5)c5cccc6oc7ccccc7c56)cc34)cccc21] | 73% |

### [Preparation Example 4] Preparation of Compound 241

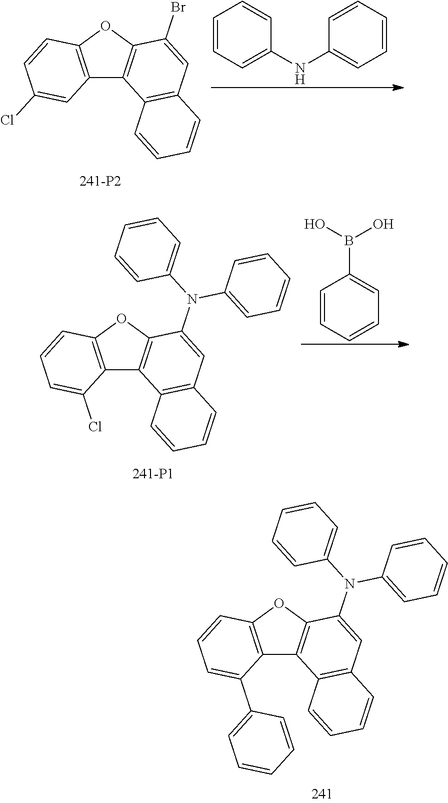

### 1) Preparation of Compound 241-P1

After Compound 241-P2 (23 g, 0.0694 mol) and diphenylamine (12.9 g, 0.0763 mol) were dissolved in 300 mL of toluene, Pd₂(dba)₃ (3.2 g, 0.0035 mol), xphos (5 g, 0.0104 mol), and NaOt-Bu (13.3 g, 0.1387 mol) were added thereto, and the resulting mixture was stirred under reflux at 50° C. for 3 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 241-P1 (18 g, 62%). 2) Preparation of Compound 241 After Compound 241-P1 (18 g, 0.0429 mol) and phenylboronic acid (6.7 g, 0.0514 mol) were dissolved in 200 mL of toluene and 50 mL of dissolved water, Pd₂(dba)₃ (2 g, 0.0021 mol), xphos (3.1 g, 0.0064 mol), and NaOH (3.4 g, 0.0857 mol) were added thereto, and the resulting mixture was stirred under reflux for 8 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 241 (15 g, 76%).

A target compound in the following Table 4 was synthesized by preparation in the same manner as in Preparation Example 4, except that Compound I, Compound J, and Compound K were used instead of 241-P2, diphenylamine, and phenylboronic acid, respectively, in Preparation Example 4.

TABLE 4

| Com- | | | | | |
|---|---|---|---|---|---|
| pound | Compound | | | | |
| No. | I | Compound J | Compound K | Target Compound | Yield |
| 250 | 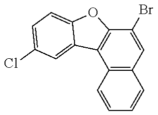 [SMILES: Clc1ccc2oc3c(Br)cc4ccccc4c3c2c1] | 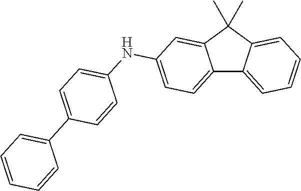 [SMILES: CC1(C)c2ccccc2-c2ccc(Nc3ccc(-c4ccccc4)cc3)cc21] | 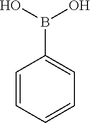 | 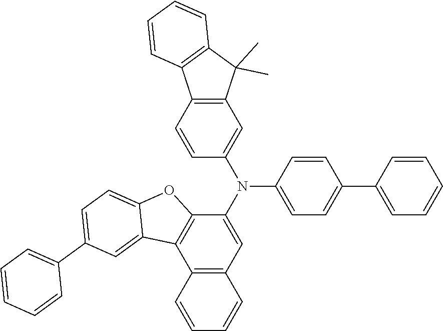 [SMILES: CC1(C)c2ccccc2-c2ccc(N(c3ccc(-c4ccccc4)cc3)c3cc4ccccc4c4c3oc3ccc(-c5ccccc5)cc34)cc21] | 81% |
| 252 | 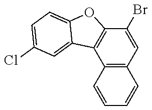 [SMILES: Clc1ccc2oc3c(Br)cc4ccccc4c3c2c1] | 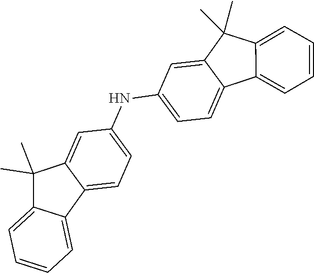 [SMILES: CC1(C)c2ccccc2-c2ccc(Nc3ccc4c(c3)C(C)(C)c3ccccc3-4)cc21] | 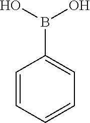 | 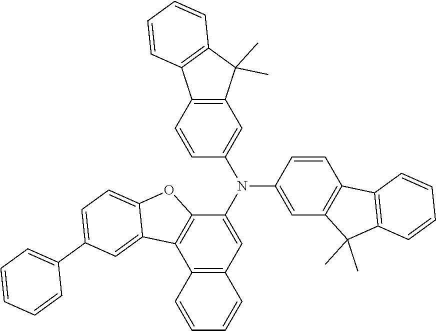 [SMILES: CC1(C)c2ccccc2-c2ccc(N(c3ccc4c(c3)C(C)(C)c3ccccc3-4)c3cc4ccccc4c4c3oc3ccc(-c5ccccc5)cc34)cc21] | 78% |
| 256 | 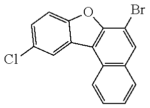 [SMILES: Clc1ccc2oc3c(Br)cc4ccccc4c3c2c1] | 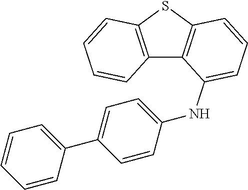 [SMILES: c1ccc(-c2ccc(Nc3cccc4sc5ccccc5c34)cc2)cc1] | 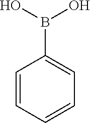 | 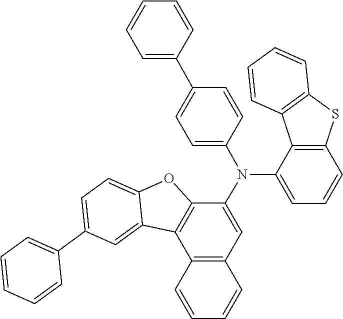 [SMILES: c1ccc(-c2ccc(N(c3cc4ccccc4c4c3oc3ccc(-c5ccccc5)cc34)c3cccc4sc5ccccc5c34)cc2)cc1] | 77% |
| 277 | 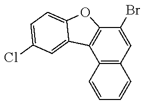 [SMILES: Clc1ccc2oc3c(Br)cc4ccccc4c3c2c1] | 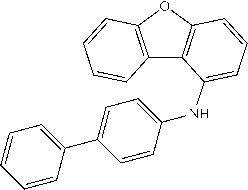 [SMILES: c1ccc(-c2ccc(Nc3cccc4oc5ccccc5c34)cc2)cc1] | 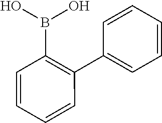 [SMILES: OB(O)c1ccccc1-c1ccccc1] | 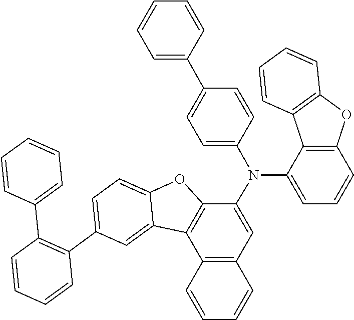 [SMILES: c1ccc(-c2ccc(N(c3cc4ccccc4c4c3oc3ccc(-c5ccccc5-c5ccccc5)cc34)c3cccc4oc5ccccc5c34)cc2)cc1] | 75% |
| 281 | 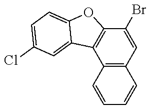 [SMILES: Clc1ccc2oc3c(Br)cc4ccccc4c3c2c1] | 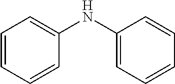 [SMILES: c1ccc(Nc2ccccc2)cc1] | 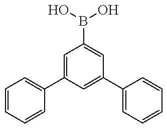 [SMILES: OB(O)c1cc(-c2ccccc2)cc(-c2ccccc2)c1] | 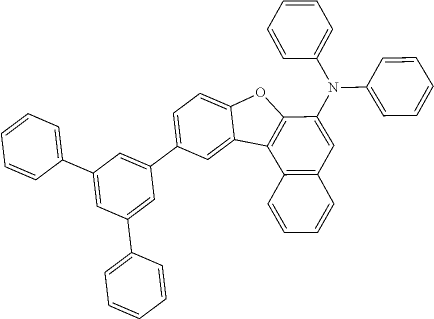 [SMILES: c1ccc(-c2cc(-c3ccccc3)cc(-c3ccc4oc5c(N(c6ccccc6)c6ccccc6)cc6ccccc6c5c4c3)c2)cc1] | 78% |
| 292 | 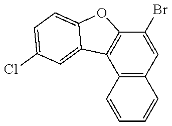 [SMILES: Clc1ccc2oc3c(Br)cc4ccccc4c3c2c1] | 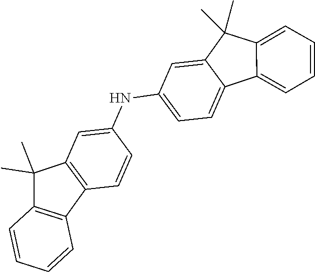 [SMILES: CC1(C)c2ccccc2-c2ccc(Nc3ccc4c(c3)C(C)(C)c3ccccc3-4)cc21] | 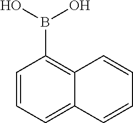 [SMILES: OB(O)c1cccc2ccccc12] | 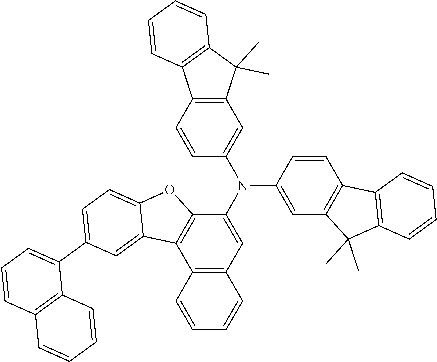 [SMILES: CC1(C)c2ccccc2-c2ccc(N(c3ccc4c(c3)C(C)(C)c3ccccc3-4)c3cc4ccccc4c4c3oc3ccc(-c5cccc6ccccc56)cc34)cc21] | 76% |
| 317 | 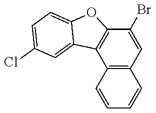 [SMILES: Clc1ccc2oc3c(Br)cc4ccccc4c3c2c1] | 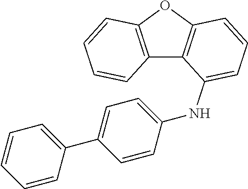 [SMILES: c1ccc(-c2ccc(Nc3cccc4oc5ccccc5c34)cc2)cc1] | 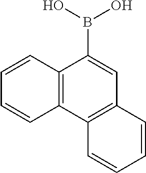 [SMILES: OB(O)c1cc2ccccc2c2ccccc12] | 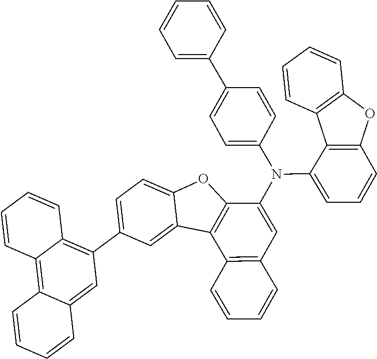 [SMILES: c1ccc(-c2ccc(N(c3cc4ccccc4c4c3oc3ccc(-c5cc6ccccc6c6ccccc56)cc34)c3cccc4oc5ccccc5c34)cc2)cc1] | 72% |
| 352 | 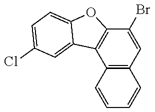 [SMILES: Clc1ccc2oc3c(Br)cc4ccccc4c3c2c1] | 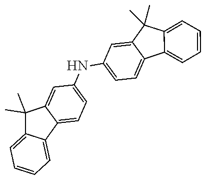 [SMILES: CC1(C)c2ccccc2-c2ccc(Nc3ccc4c(c3)C(C)(C)c3ccccc3-4)cc21] | 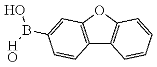 [SMILES: OB(O)c1ccc2c(c1)oc1ccccc12] | 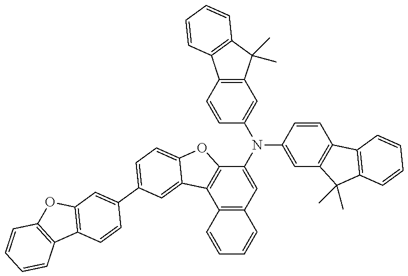 [SMILES: CC1(C)c2ccccc2-c2ccc(N(c3ccc4c(c3)C(C)(C)c3ccccc3-4)c3cc4ccccc4c4c3oc3ccc(-c5ccc6c(c5)oc5ccccc56)cc34)cc21] | 79% |
| 363 | 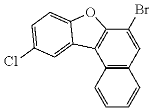 [SMILES: Clc1ccc2oc3c(Br)cc4ccccc4c3c2c1] | 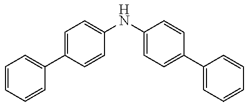 [SMILES: c1ccc(-c2ccc(Nc3ccc(-c4ccccc4)cc3)cc2)cc1] | 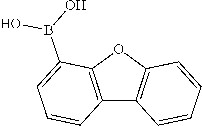 [SMILES: OB(O)c1cccc2c1oc1ccccc12] | 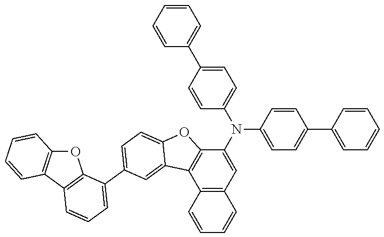 [SMILES: c1ccc(-c2ccc(N(c3ccc(-c4ccccc4)cc3)c3cc4ccccc4c4c3oc3ccc(-c5cccc6c5oc5ccccc56)cc34)cc2)cc1] | 77% |
| 376 | 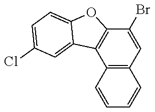 [SMILES: Clc1ccc2oc3c(Br)cc4ccccc4c3c2c1] | 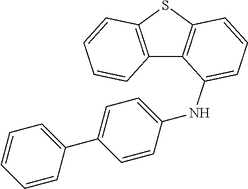 [SMILES: c1ccc(-c2ccc(Nc3cccc4sc5ccccc5c34)cc2)cc1] | 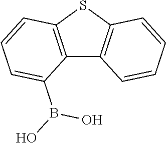 [SMILES: OB(O)c1cccc2sc3ccccc3c12] | 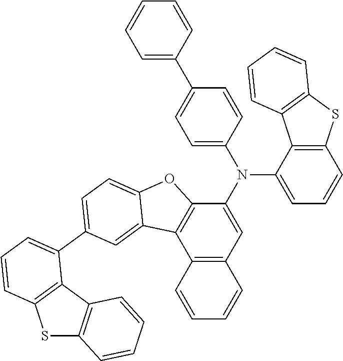 [SMILES: c1ccc(-c2ccc(N(c3cc4ccccc4c4c3oc3ccc(-c5cccc6sc7ccccc7c56)cc34)c3cccc4sc5ccccc5c34)cc2)cc1] | 76% |
| 377 | 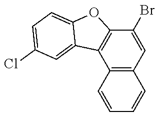 [SMILES: Clc1ccc2oc3c(Br)cc4ccccc4c3c2c1] | 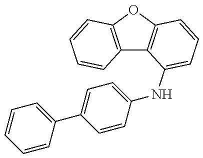 [SMILES: c1ccc(-c2ccc(Nc3cccc4oc5ccccc5c34)cc2)cc1] | 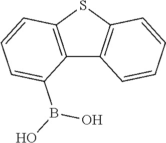 [SMILES: OB(O)c1cccc2sc3ccccc3c12] | 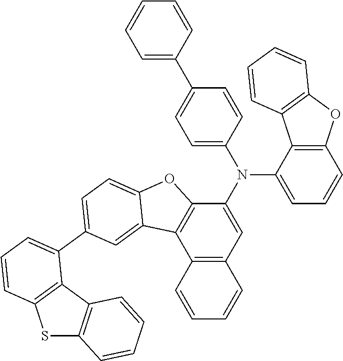 [SMILES: c1ccc(-c2ccc(N(c3cc4ccccc4c4c3oc3ccc(-c5cccc6sc7ccccc7c56)cc34)c3cccc4oc5ccccc5c34)cc2)cc1] | 81% |
| 401 | 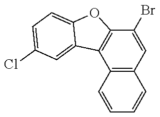 [SMILES: Clc1ccc2oc3c(Br)cc4ccccc4c3c2c1] | 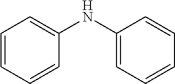 [SMILES: c1ccc(Nc2ccccc2)cc1] | 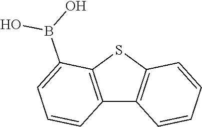 [SMILES: OB(O)c1cccc2c1sc1ccccc12] | 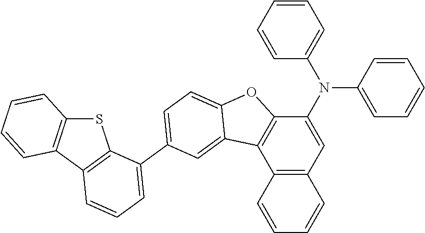 [SMILES: c1ccc(N(c2ccccc2)c2cc3ccccc3c3c2oc2ccc(-c4cccc5c4sc4ccccc45)cc23)cc1] | 79% |
| 443 | 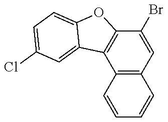 [SMILES: Clc1ccc2oc3c(Br)cc4ccccc4c3c2c1] | 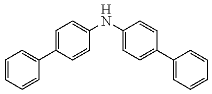 [SMILES: c1ccc(-c2ccc(Nc3ccc(-c4ccccc4)cc3)cc2)cc1] | 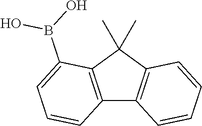 [SMILES: CC1(C)c2ccccc2-c2cccc(B(O)O)c21] | 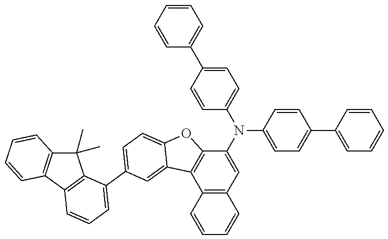 [SMILES: CC1(C)c2ccccc2-c2cccc(-c3ccc4oc5c(N(c6ccc(-c7ccccc7)cc6)c6ccc(-c7ccccc7)cc6)cc6ccccc6c5c4c3)c21] | 76% |
| 550 | 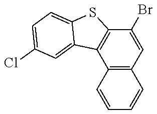 [SMILES: Clc1ccc2sc3c(Br)cc4ccccc4c3c2c1] | 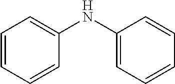 [SMILES: c1ccc(Nc2ccccc2)cc1] | 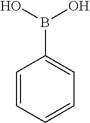 | 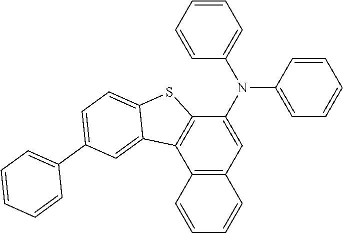 [SMILES: c1ccc(-c2ccc3sc4c(N(c5ccccc5)c5ccccc5)cc5ccccc5c4c3c2)cc1] | 77% |
| 570 | 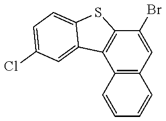 [SMILES: Clc1ccc2sc3c(Br)cc4ccccc4c3c2c1] | 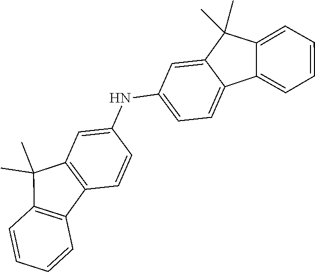 [SMILES: CC1(C)c2ccccc2-c2ccc(Nc3ccc4c(c3)C(C)(C)c3ccccc3-4)cc21] | 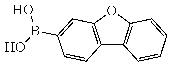 [SMILES: OB(O)c1ccc2c(c1)oc1ccccc12] | 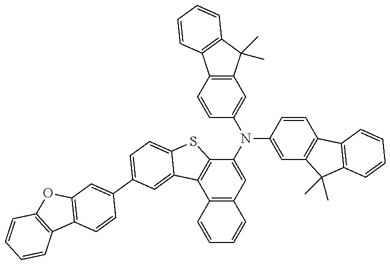 [SMILES: CC1(C)c2ccccc2-c2ccc(N(c3ccc4c(c3)C(C)(C)c3ccccc3-4)c3cc4ccccc4c4c3sc3ccc(-c5ccc6c(c5)oc5ccccc56)cc34)cc21] | 76% |
| 577 | 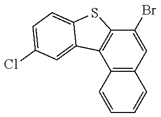 [SMILES: Clc1ccc2sc3c(Br)cc4ccccc4c3c2c1] | 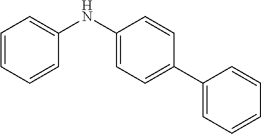 [SMILES: c1ccc(Nc2ccc(-c3ccccc3)cc2)cc1] | 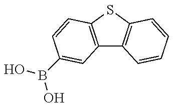 [SMILES: OB(O)c1ccc2sc3ccccc3c2c1] | 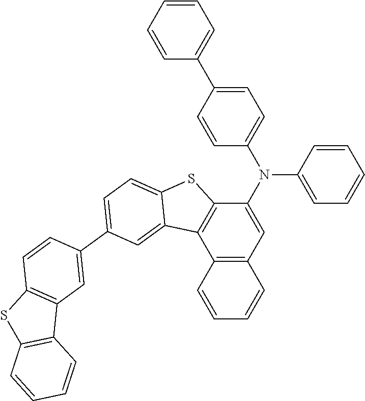 [SMILES: c1ccc(-c2ccc(N(c3ccccc3)c3cc4ccccc4c4c3sc3ccc(-c5ccc6sc7ccccc7c6c5)cc34)cc2)cc1] | 81% |
| 582 | 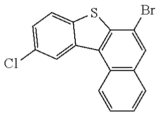 [SMILES: Clc1ccc2sc3c(Br)cc4ccccc4c3c2c1] | 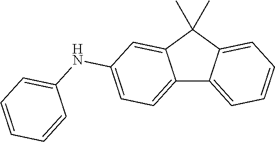 [SMILES: CC1(C)c2ccccc2-c2ccc(Nc3ccccc3)cc21] | 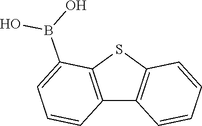 [SMILES: OB(O)c1cccc2c1sc1ccccc12] | 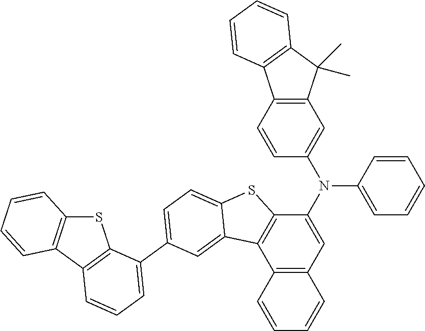 [SMILES: CC1(C)c2ccccc2-c2ccc(N(c3ccccc3)c3cc4ccccc4c4c3sc3ccc(-c5cccc6c5sc5ccccc56)cc34)cc21] | 78% |
| 583 | 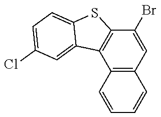 [SMILES: Clc1ccc2sc3c(Br)cc4ccccc4c3c2c1] | 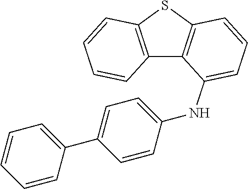 [SMILES: c1ccc(-c2ccc(Nc3cccc4sc5ccccc5c34)cc2)cc1] | 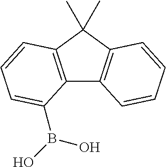 [SMILES: CC1(C)c2ccccc2-c2c(B(O)O)cccc21] | 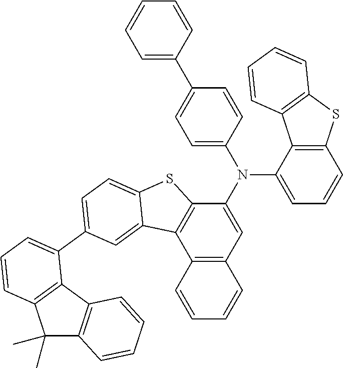 [SMILES: CC1(C)c2ccccc2-c2c(-c3ccc4sc5c(N(c6ccc(-c7ccccc7)cc6)c6cccc7sc8ccccc8c67)cc6ccccc6c5c4c3)cccc21] | 76% |

### [Preparation Example ] Preparation of Compound 451

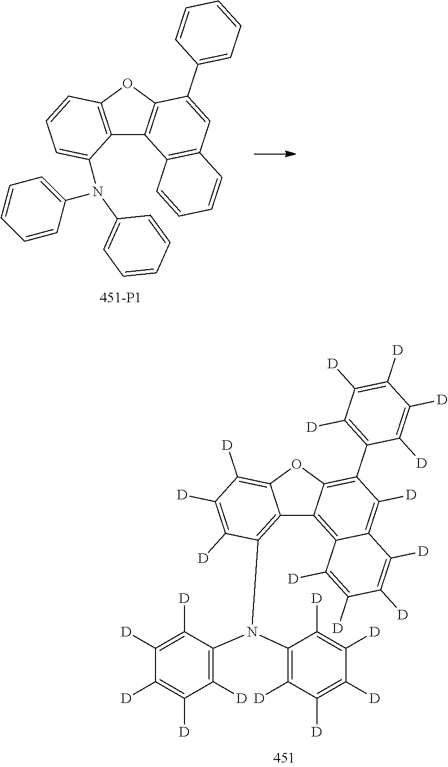 [SMILES: c1ccc(-c2cc3ccccc3c3c2oc2cccc(N(c4ccccc4)c4ccccc4)c23)cc1 ; [2H]c1c([2H])c([2H])c(-c2c([2H])c3c([2H])c([2H])c([2H])c([2H])c3c3c2oc2c([2H])c([2H])c([2H])c(N(c4c([2H])c([2H])c([2H])c([2H])c4[2H])c4c([2H])c([2H])c([2H])c([2H])c4[2H])c23)c([2H])c1[2H]]

### Preparation of Compound 451

Compound 451-P1 (10 g, 0.0217 mol), benzene-D6 (100 mL, 10 T), and triflic acid (9.6 mL, 0.1083 mol) were put into a flask and stirred at 60° C. After 4 hours, 300 mL of purified water and NaHCO₃ were added dropwise thereto to terminate the reaction. After extraction was performed using distilled water and the organic layer was dried over anhydrous MgSO₄, the solvent was removed by a rotary evaporator, and then the residue was purified by being allowed to pass through silica gel using dichloromethane and hexane as developing solvents, and Compound 451 (10 g, 95%) was obtained.

A target compound in the following Table 5 was synthesized by preparation in the same manner as in Preparation Example 5, except that Compound L was used instead of 451-P1 in Preparation Example 5.

TABLE 5

| Com- | | | |
|---|---|---|---|
| pound | | | |
| No. | Compound L | Target Compound | Yield |
| 454 | 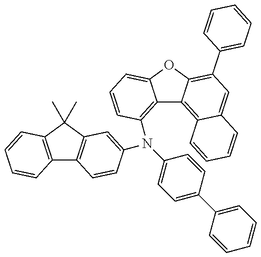 [SMILES: CC1(C)c2ccccc2-c2ccc(N(c3ccc(-c4ccccc4)cc3)c3cccc4oc5c(-c6ccccc6)cc6ccccc6c5c34)cc21] | 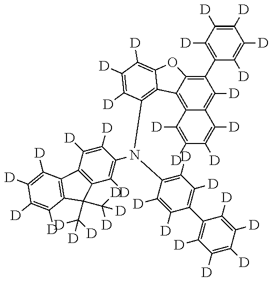 [SMILES: [2H]c1c([2H])c([2H])c(-c2c([2H])c([2H])c(N(c3c([2H])c([2H])c4c(c3[2H])C(C([2H])([2H])[2H])(C([2H])([2H])[2H])c3c([2H])c([2H])c([2H])c([2H])c3-4)c3c([2H])c([2H])c([2H])c4oc5c(-c6c([2H])c([2H])c([2H])c([2H])c6[2H])c([2H])c6c([2H])c([2H])c([2H])c([2H])c6c5c34)c([2H])c2[2H])c([2H])c1[2H]] | 97% |
| 456 | 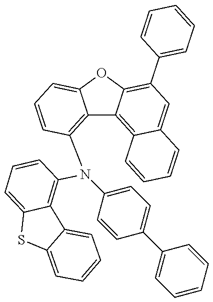 [SMILES: c1ccc(-c2ccc(N(c3cccc4sc5ccccc5c34)c3cccc4oc5c(-c6ccccc6)cc6ccccc6c5c34)cc2)cc1] | 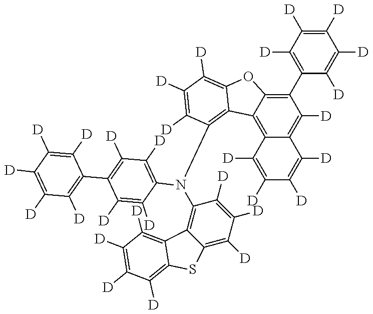 [SMILES: [2H]c1c([2H])c([2H])c(-c2c([2H])c([2H])c(N(c3c([2H])c([2H])c([2H])c4sc5c([2H])c([2H])c([2H])c([2H])c5c34)c3c([2H])c([2H])c([2H])c4oc5c(-c6c([2H])c([2H])c([2H])c([2H])c6[2H])c([2H])c6c([2H])c([2H])c([2H])c([2H])c6c5c34)c([2H])c2[2H])c([2H])c1[2H]] | 95% |
| 459 | 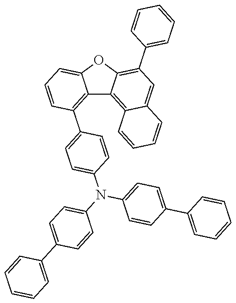 [SMILES: c1ccc(-c2ccc(N(c3ccc(-c4ccccc4)cc3)c3ccc(-c4cccc5oc6c(-c7ccccc7)cc7ccccc7c6c45)cc3)cc2)cc1] | 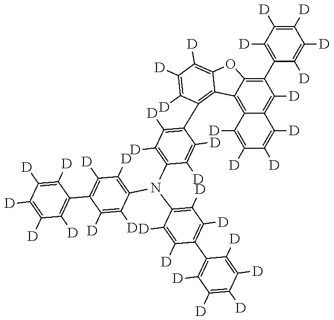 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c5oc6c(-c7c([2H])c([2H])c([2H])c([2H])c7[2H])c([2H])c7c([2H])c([2H])c([2H])c([2H])c7c6c45)c([2H])c3[2H])c([2H])c2[2H])c([2H])c1[2H]] | 96% |
| 591 | 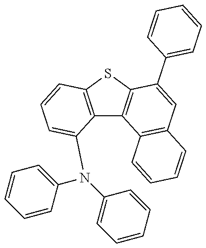 [SMILES: c1ccc(-c2cc3ccccc3c3c2sc2cccc(N(c4ccccc4)c4ccccc4)c23)cc1] | 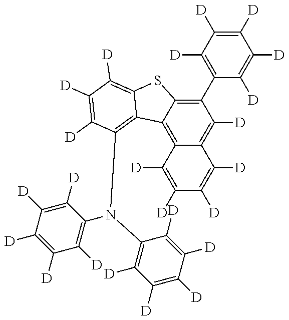 [SMILES: [2H]c1c([2H])c([2H])c(-c2c([2H])c3c([2H])c([2H])c([2H])c([2H])c3c3c2sc2c([2H])c([2H])c([2H])c(N(c4c([2H])c([2H])c([2H])c([2H])c4[2H])c4c([2H])c([2H])c([2H])c([2H])c4[2H])c23)c([2H])c1[2H]] | 96% |
| 592 | 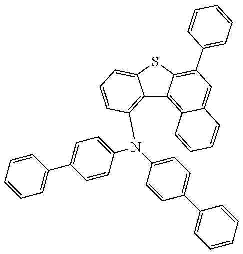 [SMILES: c1ccc(-c2ccc(N(c3ccc(-c4ccccc4)cc3)c3cccc4sc5c(-c6ccccc6)cc6ccccc6c5c34)cc2)cc1] | 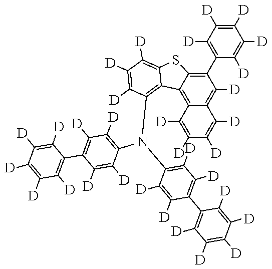 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c([2H])c4sc5c(-c6c([2H])c([2H])c([2H])c([2H])c6[2H])c([2H])c6c([2H])c([2H])c([2H])c([2H])c6c5c34)c([2H])c2[2H])c([2H])c1[2H]] | 95% |
| 595 | 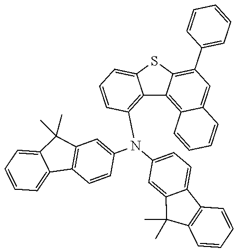 [SMILES: CC1(C)c2ccccc2-c2ccc(N(c3ccc4c(c3)C(C)(C)c3ccccc3-4)c3cccc4sc5c(-c6ccccc6)cc6ccccc6c5c34)cc21] | 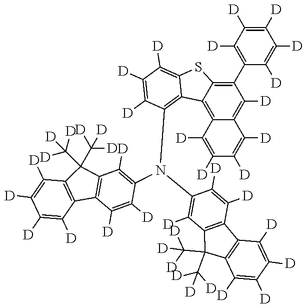 [SMILES: [2H]c1c([2H])c([2H])c(-c2c([2H])c3c([2H])c([2H])c([2H])c([2H])c3c3c2sc2c([2H])c([2H])c([2H])c(N(c4c([2H])c([2H])c5c(c4[2H])C(C([2H])([2H])[2H])(C([2H])([2H])[2H])c4c([2H])c([2H])c([2H])c([2H])c4-5)c4c([2H])c([2H])c5c(c4[2H])C(C([2H])([2H])[2H])(C([2H])([2H])[2H])c4c([2H])c([2H])c([2H])c([2H])c4-5)c23)c([2H])c1[2H]] | 94% |
| 607 | 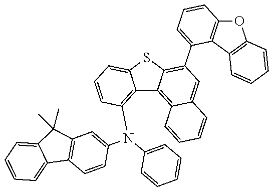 [SMILES: CC1(C)c2ccccc2-c2ccc(N(c3ccccc3)c3cccc4sc5c(-c6cccc7oc8ccccc8c67)cc6ccccc6c5c34)cc21] | 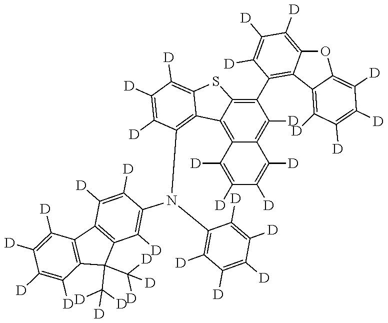 [SMILES: [2H]c1c([2H])c([2H])c(N(c2c([2H])c([2H])c3c(c2[2H])C(C([2H])([2H])[2H])(C([2H])([2H])[2H])c2c([2H])c([2H])c([2H])c([2H])c2-3)c2c([2H])c([2H])c([2H])c3sc4c(-c5c([2H])c([2H])c([2H])c6oc7c([2H])c([2H])c([2H])c([2H])c7c56)c([2H])c5c([2H])c([2H])c([2H])c([2H])c5c4c23)c([2H])c1[2H]] | 95% |

### [Preparation Example 6] Preparation of Compound 482

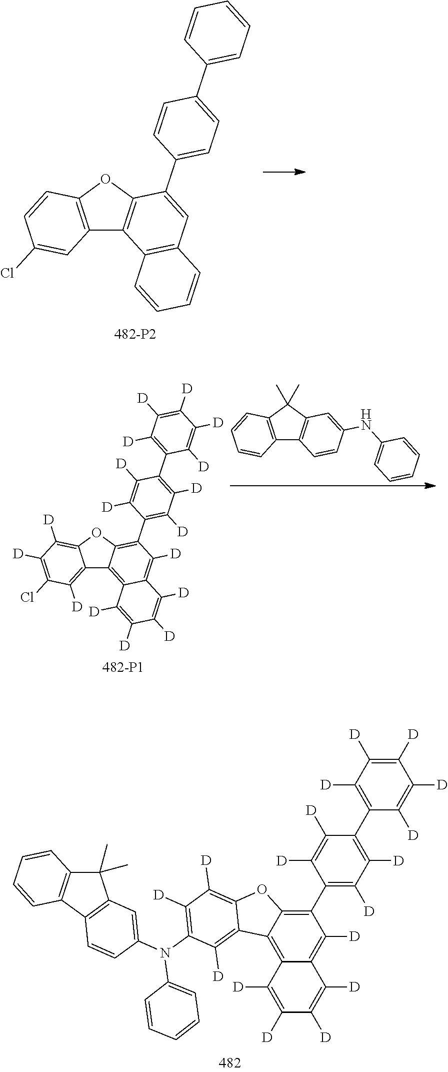

### 1) Preparation of Compound 482-P1

Compound 482-P1 (10 g, 0.0247 mol), benzene-D6 (100 mL, 10 T), and triflic acid (10.9 mL, 0.1235 mol) were put into a flask and stirred at 60° C. After 4 hours, 300 mL of purified water and NaHCO₃ were added dropwise thereto to terminate the reaction. After extraction was performed using distilled water and the organic layer was dried over anhydrous MgSO₄, the solvent was removed by a rotary evaporator, and then the residue was purified by being allowed to pass through silica gel using dichloromethane and hexane as developing solvents, and Compound 482-P1 (10 g, 96%) was obtained.

### 2) Preparation of Compound 482

After Compound 482-P1 (10 g, 0.0237 mol) and 9,9-dimethyl-N-phenyl-9H-fluoren-2-amine (7.4 g, 0.0261 mmol) were dissolved in 100 mL of toluene, Pd₂(dba)₃ (1.1 g, 0.0012 mol), xphos (1.7 g, 0.0036 mol), and t-BuONa (4.6 g, 0.0474 mol) were added thereto, and the resulting mixture was stirred under reflux for 3 hours. After the reaction was completed, MC was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 482 (8.5 g, 75%).

A target compound in the following Table 6 was synthesized by preparation in the same manner as in Preparation Example 6, except that Compound M and Compound N were used instead of 482-P2 and 9,9-dimethyl-N-phenyl-9H-fluoren-2-amine, respectively, in Preparation Example 6.

TABLE 6

| Compound | | | | |
|---|---|---|---|---|
| No. | Compound M | Compound N | Target Compound | Yield |
| 485 | 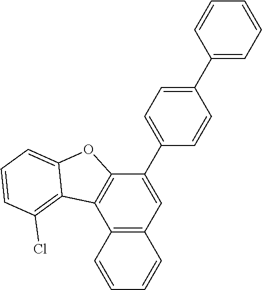 [SMILES: Clc1cccc2oc3c(-c4ccc(-c5ccccc5)cc4)cc4ccccc4c3c12] | 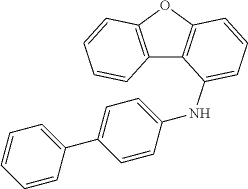 [SMILES: c1ccc(-c2ccc(Nc3cccc4oc5ccccc5c34)cc2)cc1] | 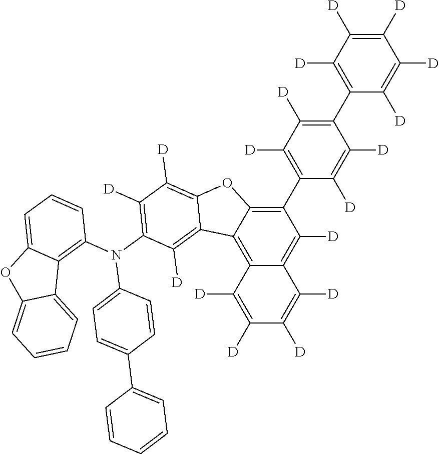 [SMILES: [2H]c1c([2H])c([2H])c(-c2c([2H])c([2H])c(-c3c([2H])c4c([2H])c([2H])c([2H])c([2H])c4c4c3oc3c([2H])c([2H])c(N(c5ccc(-c6ccccc6)cc5)c5cccc6oc7ccccc7c56)c([2H])c34)c([2H])c2[2H])c([2H])c1[2H]] | 72% |
| 489 | 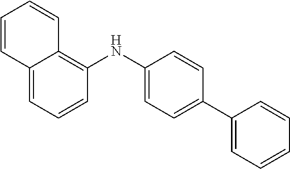 [SMILES: c1ccc(-c2ccc(Nc3cccc4ccccc34)cc2)cc1] | 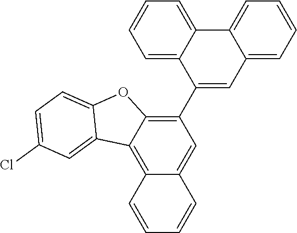 [SMILES: Clc1ccc2oc3c(-c4cc5ccccc5c5ccccc45)cc4ccccc4c3c2c1] | 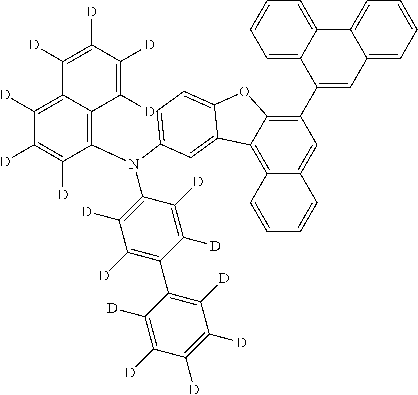 [SMILES: [2H]c1c([2H])c([2H])c(-c2c([2H])c([2H])c(N(c3ccc4oc5c(-c6cc7ccccc7c7ccccc67)cc6ccccc6c5c4c3)c3c([2H])c([2H])c([2H])c4c([2H])c([2H])c([2H])c([2H])c34)c([2H])c2[2H])c([2H])c1[2H]] | 70% |
| 490 | 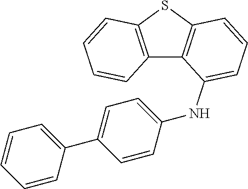 [SMILES: c1ccc(-c2ccc(Nc3cccc4sc5ccccc5c34)cc2)cc1] | 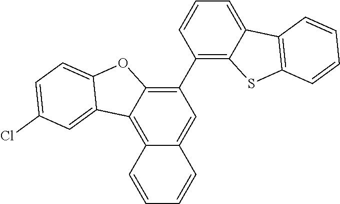 [SMILES: Clc1ccc2oc3c(-c4cccc5c4sc4ccccc45)cc4ccccc4c3c2c1] | 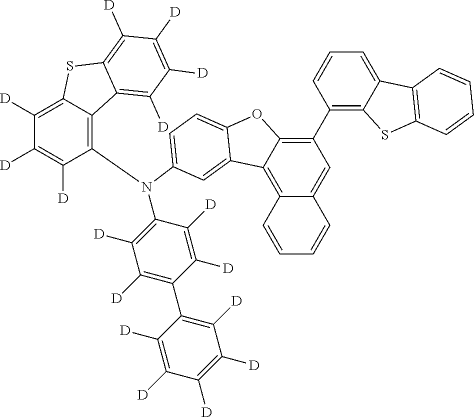 [SMILES: [2H]c1c([2H])c([2H])c(-c2c([2H])c([2H])c(N(c3ccc4oc5c(-c6cccc7c6sc6ccccc67)cc6ccccc6c5c4c3)c3c([2H])c([2H])c([2H])c4sc5c([2H])c([2H])c([2H])c([2H])c5c34)c([2H])c2[2H])c([2H])c1[2H]] | 72% |
| 622 | 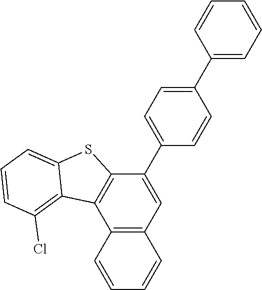 [SMILES: Clc1cccc2sc3c(-c4ccc(-c5ccccc5)cc4)cc4ccccc4c3c12] | 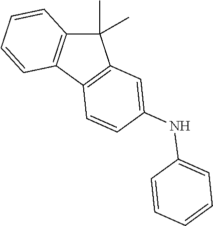 [SMILES: CC1(C)c2ccccc2-c2ccc(Nc3ccccc3)cc21] | 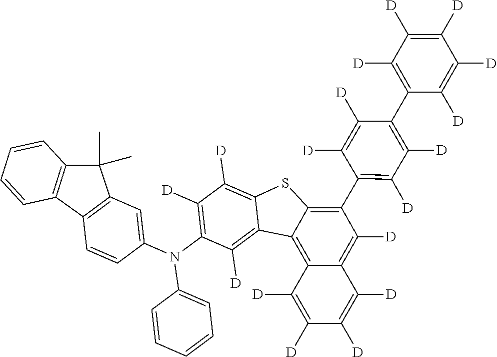 [SMILES: [2H]c1c([2H])c([2H])c(-c2c([2H])c([2H])c(-c3c([2H])c4c([2H])c([2H])c([2H])c([2H])c4c4c3sc3c([2H])c([2H])c(N(c5ccccc5)c5ccc6c(c5)C(C)(C)c5ccccc5-6)c([2H])c34)c([2H])c2[2H])c([2H])c1[2H]] | 73% |
| 631 | 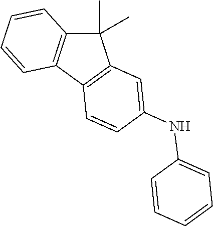 [SMILES: CC1(C)c2ccccc2-c2ccc(Nc3ccccc3)cc21] | 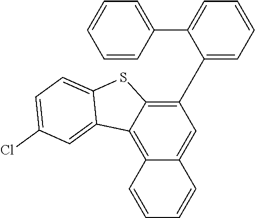 [SMILES: Clc1ccc2sc3c(-c4ccccc4-c4ccccc4)cc4ccccc4c3c2c1] | 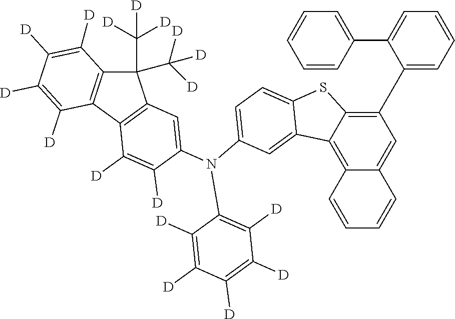 [SMILES: [2H]c1c(N(c2ccc3sc4c(-c5ccccc5-c5ccccc5)cc5ccccc5c4c3c2)c2c([2H])c([2H])c([2H])c([2H])c2[2H])cc2c(c1[2H])-c1c([2H])c([2H])c([2H])c([2H])c1C2(C([2H])([2H])[2H])C([2H])([2H])[2H]] | 70% |
| 638 | 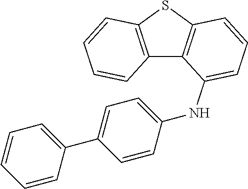 [SMILES: c1ccc(-c2ccc(Nc3cccc4sc5ccccc5c34)cc2)cc1] | 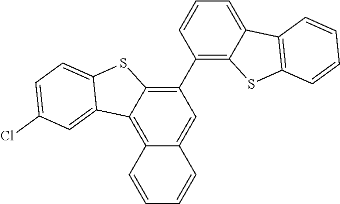 [SMILES: Clc1ccc2sc3c(-c4cccc5c4sc4ccccc45)cc4ccccc4c3c2c1] | 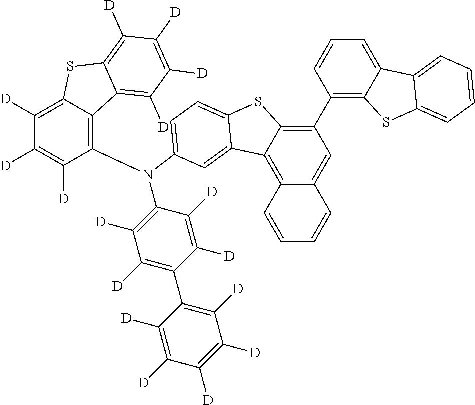 [SMILES: [2H]c1c([2H])c([2H])c(-c2c([2H])c([2H])c(N(c3ccc4sc5c(-c6cccc7c6sc6ccccc67)cc6ccccc6c5c4c3)c3c([2H])c([2H])c([2H])c4sc5c([2H])c([2H])c([2H])c([2H])c5c34)c([2H])c2[2H])c([2H])c1[2H]] | 72% |

The compound corresponding to Chemical Formula 1 was prepared in the same manner as in the Preparation Example, and the synthesis confirmation results are shown in Tables 7 and 8. Table 7 is about the measurement values of 1H NMR (CDCl₃, 300 MHz), and Table 8 is about the measurement values of field desorption mass spectrometry (FD-MS).

TABLE 7

| Compound | ¹H NMR (CDCl₃, 300 MHz) |
|---|---|
| 1 | δ = 8.02-7.98 (2H, m), 7.59-7.24 (14H, m), 7.08-7.00 |
| | (6H, m), 6.91 (1H, d) |
| 3 | δ = 8.02-7.98 (2H, m), 7.75 (4H, m), 7.55-7.37 (23H, m), |
| | 7.25 (1H, d), 6.91 (1H, d) |
| 5 | δ = 8.22-8.15 (2H, m), 8.02-7.98 (2H, m), 7.81-7.75 (3H, |
| | m), 7.63-7.37 (20H, d), 7.25 (1H, d), 6.91 (1H, d) |
| 11 | δ = 8.02-7.98 (2H, m), 7.90-7.86 (2H, m), 7.75 (2H, d), |
| | 7.59-7.37 (21H, m), 7.16 (1H, d) 6.91 (1H, d), 1.69 |
| | (6H, s) |
| 19 | δ = 8.02-7.98 (3H, m), 7.75 (2H, m) 7.55-7.37 (22H, m), |
| | 6.91 (2H, d) |
| 20 | δ = 8.02-7.98 (2H, m), 7.82 (1H, d), 7.69 (1H, d), 7.59- |
| | 7.37 (13H, m), 7.24 (4H, m) 7.08-7.00 (6H, m) |
| 29 | δ = 8.02-7.98 (2H, m), 7.75 (4H, m), 7.55-7.17 (28H, m), |
| | 6.91 (1H, d) |
| 56 | δ = 8.02-7.96 (4H, m), 7.79-7.71 (6H, m), 7.60-7.25 |
| | (18H, m) 7.11 (2H, s), 6.91 (1H, d) |
| 70 | δ = 8.02-7.96 (4H, m), 7.79-7.82 (3H, m), 7.69-7.37 |
| | (14H, m), 7.24 (4H, m), 7.08-7.00 (6H, m) |
| 85 | δ = 8.04-7.98 (5H, m), 7.90-7.86 (2H, m), 7.75 (4H, m), |
| | 7.55-7.28 (24H, m), 7.16 (1H, d), 6.91 (1H, d), 1.69 |
| | (6H, s) |
| 96 | δ = 8.04-7.98 (5H, m), 7.82-7.69 (6H, m), 7.59-7.37 |
| | (14H, m), 7.25-7.24 (8H, m), 7.08-7.00 (6H, m) |
| 101 | δ = 8.95 (1H, d), 8.50 (1H, d), 8.20 (1H, d), 8.09-7.98 |
| | (3H, m), 7.77 (1H, t), 7.59-7.52 (3H, m), 7.39-7.24 |
| | (8H, m), 7.08-6.91 (7H, m) |
| 107 | δ = 8.60 (1H, d), 9.27 (1H, s), 8.71 (1H, s), 8.37-8.27 |
| | (6H, m), 8.07-7.98 (3H, m), 7.70-7.37 (25H, m), 7.25 |
| | (1H, d), 6.91 (1H, d) |
| 113 | δ = 8.45 (1H, d), 8.03-7.90 (6H, m), 7.68-7.16 (23H, m), |
| | 6.91 (1H, d), 1.69 (6H, s) |
| 121 | δ = 8.22 (1H, s), 8.02-7.98 (2H, m), 7.59-7.39 (9H, m), |
| | 7.24 (4H, m), 7.08-6.97 (7H, m) |
| 127 | δ = 8.22 (1H, s), 8.02-7.98 (2H, m), 7.75 (6H, m), 7.60- |
| | 7.37 (25H, m), 6.97 (1H, d) |
| 128 | δ = 8.71 (1H, d), 8.33-8.22 (3H, m), 8.07-7.98 (3H, m), |
| | 7.75-7.37 (23H, m), 6.97 (1H, d) |
| 129 | δ = 9.08 (1H, d), 8.84 (1H, d), 8.27-8.22 (2H, m), 8.05- |
| | 7.90 (4H, m), 7.75-7.37 (26H, m), 6.97 (1H, d) |
| 130 | δ = 8.22 (1H, s), 8.02-7.98 (2H, m), 7.90-7.86 (2H, m), |
| | 7.55-7.00 (20H, m), 1.69 (6H, s) |
| 133 | δ = 8.22 (1H, s), 8.02-7.98 (2H, m), 7.90-7.86 (4H, m), |
| | 7.59-7.28 (17H, m), 7.16 (2H, d), 6.97 (1H, d), 1.69 |
| | (12H, s) |
| 136 | δ = 8.22 (1H, s), 8.02-7.98 (2H, m), 7.90-7.86 (2H, m), |
| | 7.59-6.97 (30H, m) |
| 137 | δ = 8.22 (1H, s), 8.02-7.85 (6H, m), 7.59-7.23 (21H, m), |
| | 7.08-6.97 (5H, m) |
| 163 | δ = 8.45 (1H, d), 8.22 (1H, s), 8.02-7.93 (3H, m), 7.75 |
| | (4H, m) 7.59-7.41 (19H, m), 7.25 (4H, m) 6.97 (1H, d) |
| 189 | δ = 8.22 (1H, s), 8.02-7.96 (5H, m), 7.79-7.75 (4H, m), |
| | 7.60-7.25 (21H, m), 6.97-6.91 (2H, m) |
| 192 | δ = 8.02-7.75 (13H, m), 7.60-7.37 (26H, m) |
| 193 | δ = 8.02-7.75 (13H, m), 7.60-7.37 (23H, m), 7.27-7.17 |
| | (3H, m) |
| 232 | δ = 8.22 (1H, s), 8.08-7.98 (5H, m), 7.90-7.86 (4H, m), |
| | 7.59-7.51 (7H, m), 7.39-7.28 (9H, m), 7.16 (2H, d), |
| | 6.91 (1H, d), 1.69 (12H, s) |
| 236 | δ = 8.55 (1H, d), 8.45 (2H, d), 8.32 (1H, d), 8.22 (1H, |
| | s), 8.02-8.93 (4H, m), 7.75-7.70 (3H, m), 7.59-7.37 |
| | (18H, m), 6.97 (1H, d) |
| 238 | δ = 8.18 (1H, s), 8.02-7.98 (2H, m), 7.90-7.68 (6H, m), |
| | 7.59-7.55 (5H, m), 7.39-7.37 (4H, m), 7.28-7.24 (5H, |
| | m), 7.08-7.00 (6H, m), 1.69 (12H, s) |
| 240 | δ = 8.02-7.98 (3H, m), 7.90-7.75 (8H, m), 7.55-7.28 |
| | (23H, m), 7.16 (1H, d), 1.69 (12H, s) |
| 241 | δ = 8.02-7.98 (2H, m), 7.79-7.75 (5H, m), 7.59-7.41 (5H, |
| | m), 7.24-7.21 (5H, m), 7.08-7.00 (6H, m) |
| 250 | δ = 8.02-7.98 (2H, m), 7.90-7.75 (9H, m), 7.55-7.21 |
| | (18H, m), 1.69 (6H, s) |
| 252 | δ = 8.02-7.98 (2H, m), 7.90-7.79 (9H, m), 7.59-7.28 (13H, |
| | m), 7.21-7.16 (3H, m), 1.69 (6H, s) |
| 256 | δ = 8.45 (1H, d), 8.02-7.75 (10H, m), 7.59-7.37 (17H, |
| | m), 7.21 (1H, s) |
| 277 | δ = 8.02-7.79 (12H, m), 7.60-7.21 (21H, m), 6.91 (1H, d) |
| 281 | δ = 8.04-7.98 (5H, m), 7.88-7.75 (7H, m), 7.59-7.41 (8H, |
| | m), 7.24-7.21 (5H, m), 7.08-7.00 (6H, m) |
| 292 | δ = 8.95 (1H, d), 8.50 (1H, d), 8.20 (1H, d), 8.09-7.98 |
| | (3H, m), 7.90-7.77 (8H, m), 7.59-7.52 (5H, m), 7.39- |
| | 7.16 (10H, m), 1.69 (12H, s) |
| 317 | δ = 9.08 (1H, d), 8.84 (1H, d), 8.27 (1H, d), 8.02-7.21 |
| | (29H, m), 6.91 (1H, d) |
| 352 | δ = 8.03-7.98 (4H, m), 7.90-7.76 (9H, m), 7.59-7.54 (5H, |
| | m), 7.39-7.28 (8H, m), 7.21-7.16 (3H, m), 1.69 (12H, s) |
| 363 | δ = 8.08-7.98 (5H, m), 7.88-7.75 (7H, m), 7.55-7.31 |
| | (20H, m), 7.21 (1H, s) |
| 376 | δ = 8.45 (2H, d), 8.03-7.37 (28H, m), 7.21 (1H, s) |
| 377 | δ = 8.45 (1H, d), 8.03-7.21 (29H, m), 6.91 (1H, d) |
| 401 | δ = 8.55 (1H, d), 8.55 (1H, d), 8.32 (1H, d), 8.02-7.49 |
| | (11H, m), 7.24-7.21 (5H, m), 7.08-7.00 (6H, m), |
| 443 | δ = 8.02-7.98 (3H, m), 7.90-7.68 (9H, m), 7.59-7.37 |
| | (19H, m), 7.28-7.21 (2H, m), 1.69 (6H, s) |
| 451 | No proton |
| 454 | No proton |
| 456 | No proton |
| 459 | No proton |
| 482 | δ = 7.90-7.86 (2H, m), 7.55 (1H, d), 7.38-7.16 (6H, m), |
| | 7.08-7.00 (3H, m), 1.69 (6H, s) |
| 485 | δ = 7.98 (1H, d), 7.75 (2H, d), 7.55-7.31 (12H, m), 6.91 |
| | (1H, d) |
| 489 | δ = 9.08 (1H, d), 8.84 (1H, d), 8.27-8.22 (2H, m), 8.05- |
| | 7.90 (4H, m), 7.70-739 (8H, m), 6.97 (1H, d) |
| 490 | δ = 8.55 (1H, d), 8.45 (1H, d), 8.32 (1H, d), 8.22 (1H, |
| | s), 8.02-7.93 (3H, m), 7.70-7.39 (7H, m), 6.97 (1H, d) |
| 501 | δ = 8.02-7.98 (2H, m), 7.59-7.41 (11H, m), 7.24 (4H, m), |
| | 7.08-7.00 (6H, m) |
| 502 | δ = 8.02-7.98 (2H, m), 7.90-7.86 (2H, m), 7.75 (2H, d), |
| | 7.59-7.28 (22H, m), 7.16 (1H, d), 1.69 (6H, s) |
| 512 | δ = 8.02-7.79 (8H, m), 7.60-7.16 (18H, m), 7.08 (3H, m), |
| | 1.69 (6H, s) |
| 522 | δ = 8.02-7.98 (3H, m), 7.90-7.75 (7H, m), 7.59-7.28 |
| | (20H, m), 7.16 (2H, d), 1.69 (6H, s) |
| 525 | δ = 8.02-7.95 (3H, m), 7.85 (1H, d), 7.59-7.41 (9H, m), |
| | 7.24 (4H, m), 7.08-7.00 (6H, m) |
| 528 | δ = 8.45 (1H, d), 8.02-7.93 (4H, m), 7.85 (1H, d), 7.75 |
| | (2H, m), 7.59-7.37 (21H, m) |
| 539 | δ = 8.04-7.95 (6H, m), 7.85 (1H, d), 7.75 (8H, m), 7.55- |
| | 7.37 (21H, m), 7.27 (1H, s), 7.18-7.17 (2H, m) |
| 548 | δ = 8.02-7.82 (8H, m), 7.69 (1H, d), 7.59-7.51 (6H, m), |
| | 7.41-7.24 (9H, m), 7.08-7.00 (3H, m), 1.69 (6H, s) |
| 549 | δ = 8.02-7.85 (6H, m), 7.78-7.75 (3H, m), 7.59-7.28 |
| | (21H, m), 6.91 (1H, d), 1.69 (6H, s) |
| 550 | δ = 8.12 (2H, m), 8.02-7.98 (3H, m), 7.75 (2H, m), 7.59 |
| | (2H, m), 7.49-7.41 (3H, m), 7.24 (4H, m), 7.08-7.00 |
| | (6H, m), 6.77 (1H, s) |
| 570 | δ = 8.12 (2H, m), 8.03-7.98 (5H, m), 7.90-7.76 (6H, m), |
| | 7.59-7.54 (5H, m), 7.39-7.28 (8H, m), 7.16 (2H, m), |
| | 6.77 (1H, s), 1.69 (12H, s) |
| 577 | δ = 8.45 (1H, d), 8.12 (4H, m), 8.02-7.98 (3H, m), 7.75 |
| | (2H, m), 7.56-7.37 (11H, m), 7.24 (2H, m), 7.08-7.00 |
| | (3H, m), 6.77 (1H, s) |
| 582 | δ = 8.55 (1H, d), 8.45 (1H, d), 8.32 (1H, d), 8.12 (2H, |
| | m), 8.02-7.90 (6H, m), 7.70 (1H, m), 7.59-7.49 (5H, m), |
| | 7.38-7.16 (6H, m), 7.08-7.00 (3H, m), 6.77 (1H, s), |
| | 1.69 (6H, s) |
| 583 | δ = 8.45 (1H, d), 8.12 (2H, m), 8.02-7.90 (H, m), 7.78- |
| | 7.75 (3H, m), 7.65-7.37 (18H, m), 7.28 (1H, m), 6.77 |
| | (1H, s), 1.69 (6H, s) |
| 591 | No proton |
| 592 | No proton |
| 595 | No proton |
| 607 | No proton |
| 622 | δ = 7.90-7.86 (2H, m), 7.55 (1H, d), 7.38-7.16 (6H, m), |
| | 7.08-7.00 (3H, m), 1.69 (6H, s) |
| 631 | δ = 7.98-7.95 (5H, m), 7.85-7.79 (3H, m), 7.60-7.41 (9H, |
| | m) |
| 638 | δ = 8.55 (1H, d), 8.45 (1H, d), 8.32 (1H, d), 8.02-7.85 |
| | (5H, m), 7.70 (1H, m), 7.59-7.41 (6H, m) |

TABLE 8

| Compound | FD-MS |
|---|---|
| 1 | m/z = 461.18 |
| | (C34H23NO = 461.56) |
| 3 | m/z = 613.24 |
| | (C46H31NO = 613.76) |
| 5 | m/z = 587.22 |
| | (C44H29NO = 587.72) |
| 11 | m/z = 653.27 |
| | (C49H35NO = 653.82) |
| 19 | m/z = 627.22 |
| | (C46H29NO2 = 627.74) |
| 20 | m/z = 537.21 |
| | (C40H27NO = 537.66) |
| 29 | m/z = 689.27 |
| | (C52H35NO = 689.86) |
| 56 | m/z = 637.24 |
| | (C48H31NO = 637.78) |
| 70 | m/z = 613.24 |
| | (C46H31NO = 613.76) |
| 85 | m/z = 729.30 |
| | (C55H39NO = 729.92) |
| 96 | m/z = 765.30 |
| | (C58H39NO = 765.96) |
| 101 | m/z = 511.19 |
| | (C38H25NO = 511.61) |
| 107 | m/z = 837.30 |
| | (C64H39NO = 838.02) |
| 113 | m/z = 759.26 |
| | (C55H37NOS = 759.97) |
| 121 | m/z = 461.18 |
| | (C34H23NO = 461.56) |
| 127 | m/z = 689.27 |
| | (C52H35NO = 689.86) |
| 128 | m/z = 687.26 |
| | (C52H33NO = 687.84) |
| 129 | m/z = 713.27 |
| | (C54H35NO = 713.88) |
| 130 | m/z = 577.24 |
| | (C43H31NO = 577.73) |
| 133 | m/z = 693.30 |
| | (C52H39NO = 693.89) |
| 136 | m/z = 701.27 |
| | (C53H35NO = 701.87) |
| 137 | m/z = 699.26 |
| | (C53H33NO = 699.85) |
| 163 | m/z = 719.23 |
| | (C52H33NOS = 719.90) |
| 189 | m/z = 703.25 |
| | (C52H33NO2 = 703.84) |
| 192 | m/z = 765.30 |
| | (C58H39NO = 765.96) |
| 193 | m/z = 765.30 |
| | (C58H39NO = 765.96) |
| 232 | m/z = 783.31 |
| | (C58H41NO2 = 783.97) |
| 236 | m/z = 749.18 |
| | (C52H31NOS2 = 749.95) |
| 238 | m/z = 653.27 |
| | (C49H35NO = 653.82) |
| 240 | m/z = 845.37 |
| | (C64H47NO = 846.09) |
| 241 | m/z = 461.18 |
| | (C34H23NO = 461.56) |
| 250 | m/z = 653.27 |
| | (C49H35NO = 653.82) |
| 252 | m/z = 693.30 |
| | (C52H39NO = 693.89) |
| 256 | m/z = 643.20 |
| | (C46H29NOS = 643.80) |
| 277 | m/z = 703.25 |
| | (C52H33NO2 = 703.84) |
| 281 | m/z = 613.24 |
| | (C46H31NO = 613.76) |
| 292 | m/z = 743.32 |
| | (C56H41NO = 743.95) |
| 317 | m/z = 727.25 |
| | (C54H33NO2 = 727.86) |
| 352 | m/z = 783.31 |
| | (C58H41NO2 = 783.97) |
| 363 | m/z = 703.25 |
| | (C52H33NO2 = 703.84) |
| 376 | m/z = 749.18 |
| | (C52H31NOS2 = 749.95) |
| 377 | m/z = 733.21 |
| | (C52H31NO2S = 733.88) |
| 401 | m/z = 567.17 |
| | (C40H25NOS = 567.71) |
| 443 | m/z = 729.30 |
| | (C55H39NO = 729.92) |
| 451 | m/z = 484.32 |
| | (C34D23NO = 484.70) |
| 454 | m/z = 688.49 |
| | (C49D35NO = 689.04) |
| 456 | m/z = 672.38 |
| | (C46D29NOS = 672.98) |
| 459 | m/z = 724.49 |
| | (C52D35NO = 725.07) |
| 482 | m/z = 670.38 |
| | (C49H18D17NO = 670.93) |
| 485 | m/z = 720.36 |
| | (C52H16D17NO2 = 720.94) |
| 489 | m/z = 703.36 |
| | (C52H17D16NO = 703.94) |
| 490 | m/z = 765.29 |
| | (C52H15D16NOS2 = 766.04) |
| 501 | m/z = 477.16 |
| | (C34H23NS = 477.63) |
| 502 | m/z = 669.25 |
| | (C49H35NS = 669.89) |
| 512 | m/z = 669.25 |
| | (C49H35NS = 669.89) |
| 522 | m/z = 759.26 |
| | (C55H37NOS = 759.97) |
| 525 | m/z = 477.16 |
| | (C34H23NS = 477.63) |
| 528 | m/z = 659.17 |
| | (C46H29NS2 = 659.86) |
| 539 | m/z = 781.28 |
| | (C58H39NS = 782.02) |
| 548 | m/z = 683.23 |
| | (C49H33NOS = 683.87) |
| 549 | m/z = 759.26 |
| | (C55H3 7NOS = 759.97) |
| 550 | m/z = 477.16 |
| | (C34H23NS = 477.63) |
| 570 | m/z = 799.29 |
| | (C58H41NOS = 800.03) |
| 577 | m/z = 659.17 |
| | (C46H29NS2 = 659.86) |
| 582 | m/z = 699.21 |
| | (C49H33NS2 = 699.93) |
| 583 | m/z = 775.24 |
| | (C55H37NS2 = 776.03) |
| 591 | m/z = 500.30 |
| | (C34D23NS = 500.77) |
| 592 | m/z = 660.41 |
| | (C46D31NS = 661.01) |
| 595 | m/z = 748.53 |
| | (C52D39NS = 749.19) |
| 607 | m/z = 716.44 |
| | (C49D33NOS = 717.07) |
| 622 | m/z = 686.36 |
| | (C49H18D17NS = 686.99) |
| 631 | m/z = 500.30 |
| | (C34D23NS = 500.77) |
| 638 | m/z = 708.34 |
| | (C51H20D15NS = 709.00) |

### <Experimental Example 1>—Manufacture of Organic Light Emitting Device

### (1) Manufacture of Organic Light Emitting Device

A glass substrate, in which ITO was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

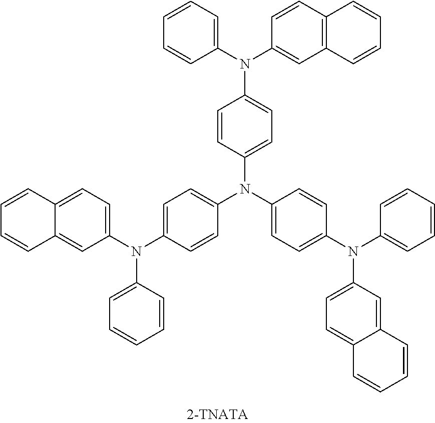 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

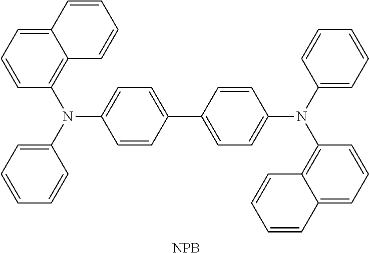 [SMILES: c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited by depositing a compound of 9-[4-(4,6-diphenyl-1,3,5-triazin-2-yl)phenyl]-9′-phenyl-3,3′-Bi-9H-carbazole as a host to have a thickness of 400 Å and doping the deposited layer with a green phosphorescent dopant Ir(ppy)₃ at 7%. Thereafter, BCP as a hole blocking layer was deposited to have a thickness of 60 Å, and Alq₃ as an electron transport layer was deposited to have a thickness of 200 Å thereon. Finally, an organic light emitting device (hereinafter, referred to as Comparative Example 1) was manufactured by depositing lithium fluoride (LiF) to have a thickness of 10 Å on the electron transport layer to form an electron injection layer, and then depositing an aluminum (Al) negative electrode to have a thickness of 1,200 Å on the electron injection layer to form a negative electrode.

Meanwhile, all the organic compounds required for manufacturing an organic light emitting device were subjected to vacuum sublimed purification under 10⁻⁶ to 10⁻⁸ torr for each material, and used for the manufacture of the organic light emitting device.

In addition, organic light emitting devices were manufactured in the same manner as in Comparative Example 1, except that the compounds described in the following Table 9 were used instead of Compound NPB used when the hole transport layer was formed in Comparative Example 1.

### (2) Driving Voltage and Light Emitting Efficiency of Organic Light Emitting Device

For the organic light emitting devices manufactured as above, electroluminescent light emission (EL) characteristics were measured using M7000 manufactured by McScience Inc., and with the measurement results, a service life T₉₅ (unit: h, hour), which was the time when the luminance became 95% compared to the initial luminance when the standard luminance was 6,000 cd/m², was measured using a service life measurement apparatus (M6000) manufactured by McScience Inc.

The characteristics of the organic light emitting devices of the present invention shown with the measurement results are shown in the following Table 9.

TABLE 9

| | | Driving | | Service |
|---|---|---|---|---|
| | | voltage | Efficiency | life |
| | Compound | (V) | (cd/A) | (T₉₅) |
| Comparative | NPB | 5.34 | 84.22 | 113 |
| Example 1 | | | | |
| Comparative | Comparative | 5.59 | 89.94 | 98 |
| Example 2 | Compound A | | | |
| Comparative | Comparative | 5.48 | 96.27 | 95 |
| Example 3 | Compound B | | | |
| Comparative | Comparative | 5.89 | 87.80 | 76 |
| Example 4 | Compound C | | | |
| Comparative | Comparative | 6.12 | 85.47 | 99 |
| Example 5 | Compound D | | | |
| Comparative | Comparative | 6.02 | 94.55 | 90 |
| Example 6 | Compound E | | | |
| Comparative | Comparative | 5.76 | 95.49 | 105 |
| Example 7 | Compound F | | | |
| Comparative | Comparative | 5.99 | 92.18 | 111 |
| Example 8 | Compound G | | | |
| Comparative | Comparative | 6.20 | 89.33 | 88 |
| Example 9 | Compound H | | | |
| Comparative | Comparative | 5.79 | 90.16 | 79 |
| Example 10 | Compound I | | | |
| Comparative | Comparative | 5.88 | 88.47 | 106 |
| Example 11 | Compound J | | | |
| Comparative | Comparative | 5.38 | 92.22 | 99 |
| Example 12 | Compound K | | | |
| Comparative | Comparative | 5.69 | 89.90 | 89 |
| Example 13 | Compound L | | | |
| Comparative | Comparative | 5.44 | 90.13 | 104 |
| Example 14 | Compound M | | | |
| Comparative | Comparative | 6.07 | 88.84 | 92 |
| Example 15 | Compound N | | | |
| Example 1 | Compound 1 | 4.17 | 120.45 | 132 |
| Example 2 | Compound 3 | 4.31 | 118.44 | 121 |
| Example 3 | Compound 5 | 4.16 | 123.94 | 139 |
| Example 4 | Compound 11 | 4.22 | 122.11 | 144 |
| Example 5 | Compound 19 | 4.14 | 114.53 | 140 |
| Example 6 | Compound 20 | 4.09 | 116.44 | 131 |
| Example 7 | Compound 29 | 4.10 | 117.32 | 125 |
| Example 8 | Compound 56 | 4.13 | 115.91 | 136 |
| Example 9 | Compound 70 | 4.25 | 120.05 | 136 |
| Example 10 | Compound 85 | 4.05 | 117.38 | 141 |
| Example 11 | Compound 96 | 4.03 | 116.88 | 138 |
| Example 12 | Compound 101 | 4.21 | 119.70 | 135 |
| Example 13 | Compound 107 | 4.08 | 115.10 | 136 |
| Example 14 | Compound 113 | 4.01 | 118.35 | 142 |
| Example 15 | Compound 121 | 4.12 | 120.35 | 139 |
| Example 16 | Compound 127 | 4.02 | 116.38 | 122 |
| Example 17 | Compound 128 | 4.08 | 120.11 | 137 |
| Example 18 | Compound 129 | 4.10 | 119.33 | 138 |
| Example 19 | Compound 130 | 3.99 | 118.21 | 125 |
| Example 20 | Compound 133 | 3.96 | 114.41 | 140 |
| Example 21 | Compound 136 | 4.04 | 116.95 | 130 |
| Example 22 | Compound 137 | 4.11 | 120.46 | 136 |
| Example 23 | Compound 163 | 3.95 | 121.17 | 138 |
| Example 24 | Compound 189 | 4.07 | 120.11 | 130 |
| Example 25 | Compound 192 | 4.17 | 121.19 | 128 |
| Example 26 | Compound 193 | 4.01 | 120.10 | 125 |
| Example 27 | Compound 232 | 3.94 | 115.74 | 132 |
| Example 28 | Compound 236 | 4.03 | 119.76 | 122 |
| Example 29 | Compound 238 | 4.11 | 116.33 | 135 |
| Example 30 | Compound 240 | 4.06 | 115.96 | 136 |
| Example 31 | Compound 241 | 3.99 | 121.31 | 142 |
| Example 32 | Compound 250 | 4.22 | 120.52 | 137 |
| Example 33 | Compound 252 | 4.15 | 120.21 | 131 |
| Example 34 | Compound 256 | 4.17 | 120.46 | 130 |
| Example 35 | Compound 277 | 4.05 | 121.67 | 132 |
| Example 36 | Compound 281 | 4.09 | 120.75 | 124 |
| Example 37 | Compound 292 | 3.96 | 116.45 | 136 |
| Example 38 | Compound 317 | 4.12 | 115.35 | 131 |
| Example 39 | Compound 352 | 3.97 | 113.15 | 134 |
| Example 40 | Compound 363 | 4.16 | 121.19 | 141 |
| Example 41 | Compound 376 | 4.07 | 116.54 | 143 |
| Example 42 | Compound 377 | 4.10 | 114.85 | 131 |
| Example 43 | Compound 401 | 3.94 | 118.21 | 133 |
| Example 44 | Compound 443 | 4.18 | 120.14 | 132 |
| Example 45 | Compound 451 | 4.07 | 117.51 | 135 |
| Example 46 | Compound 454 | 4.06 | 120.36 | 126 |
| Example 47 | Compound 456 | 4.08 | 115.35 | 124 |
| Example 48 | Compound 459 | 3.95 | 117.38 | 142 |
| Example 49 | Compound 482 | 4.11 | 118.11 | 123 |
| Example 50 | Compound 485 | 4.13 | 118.78 | 168 |
| Example 51 | Compound 489 | 4.11 | 119.62 | 172 |
| Example 52 | Compound 490 | 4.06 | 115.96 | 136 |
| Example 53 | Compound 501 | 3.99 | 121.31 | 142 |
| Example 54 | Compound 502 | 4.22 | 120.52 | 137 |
| Example 55 | Compound 512 | 4.15 | 120.21 | 131 |
| Example 56 | Compound 522 | 4.17 | 120.46 | 130 |
| Example 57 | Compound 525 | 4.05 | 121.67 | 132 |
| Example 58 | Compound 528 | 4.09 | 120.75 | 124 |
| Example 59 | Compound 539 | 3.96 | 116.45 | 136 |
| Example 60 | Compound 548 | 3.95 | 121.17 | 138 |
| Example 61 | Compound 549 | 4.07 | 120.11 | 130 |
| Example 62 | Compound 550 | 4.17 | 121.19 | 128 |
| Example 63 | Compound 570 | 4.01 | 120.10 | 125 |
| Example 64 | Compound 577 | 3.94 | 115.74 | 132 |
| Example 65 | Compound 582 | 4.03 | 119.76 | 122 |
| Example 66 | Compound 583 | 4.11 | 116.33 | 135 |
| Example 67 | Compound 591 | 4.06 | 115.96 | 136 |
| Example 68 | Compound 592 | 3.97 | 113.15 | 134 |
| Example 69 | Compound 595 | 4.16 | 121.19 | 141 |
| Example 70 | Compound 607 | 4.07 | 116.54 | 143 |
| Example 71 | Compound 622 | 4.10 | 114.85 | 131 |
| Example 72 | Compound 631 | 3.94 | 118.21 | 133 |
| Example 73 | Compound 638 | 4.18 | 120.14 | 132 |

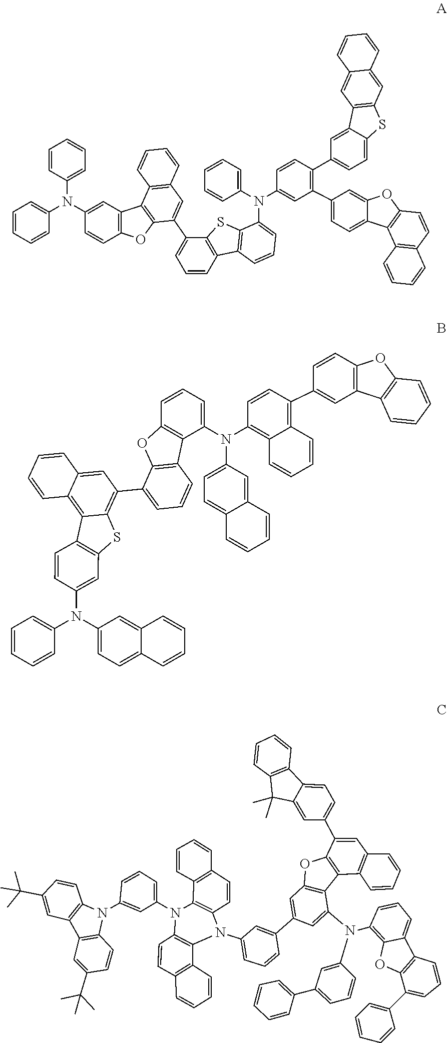

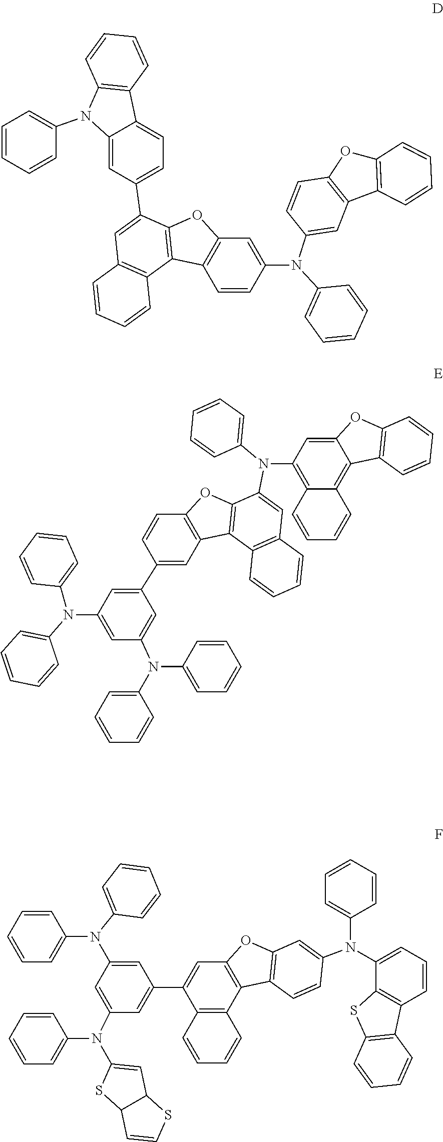 [SMILES: c1ccc(N(c2ccc3c(c2)oc2c(-c4ccc5c6ccccc6n(-c6ccccc6)c5c4)cc4ccccc4c23)c2ccc3oc4ccccc4c3c2)cc1 ; c1ccc(N(c2ccccc2)c2cc(-c3ccc4oc5c(N(c6ccccc6)c6cc7oc8ccccc8c7c7ccccc67)cc6ccccc6c5c4c3)cc(N(c3ccccc3)c3ccccc3)c2)cc1 ; C1=CC2SC(N(c3ccccc3)c3cc(-c4cc5oc6cc(N(c7ccccc7)c7cccc8c7sc7ccccc78)ccc6c5c5ccccc45)cc(N(c4ccccc4)c4ccccc4)c3)=CC2S1]

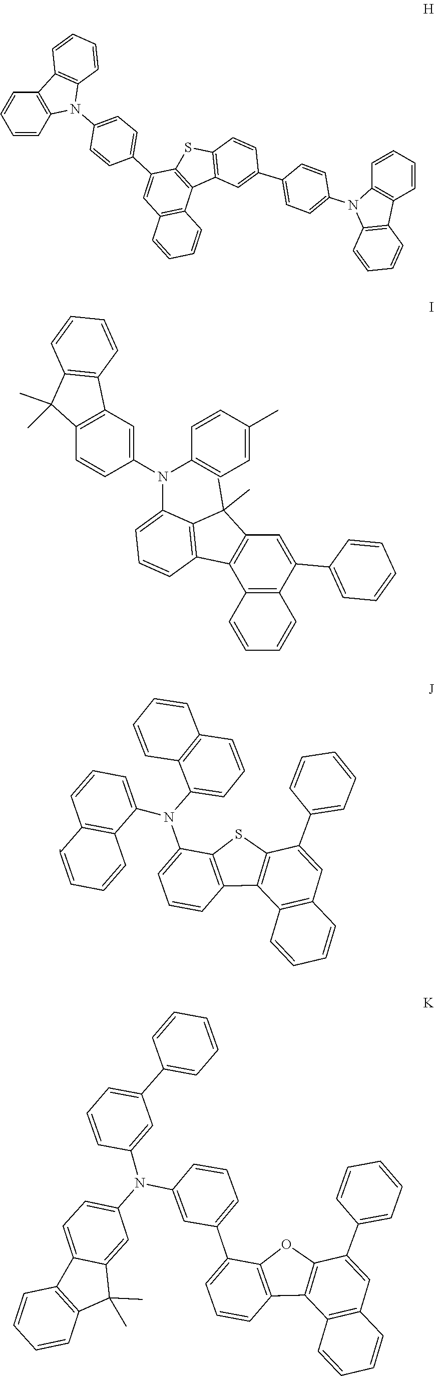 [SMILES: c1ccc2c(c1)cc(-c1ccc(-n3c4ccccc4c4ccccc43)cc1)c1sc3ccc(-c4ccc(-n5c6ccccc6c6ccccc65)cc4)cc3c12 ; Cc1ccc(N(c2ccc3c(c2)-c2ccccc2C3(C)C)c2cccc3c2C(C)(C)c2cc(-c4ccccc4)c4ccccc4c2-3)cc1 ; Cc1cccc2c(N(c3cccc4ccccc34)c3cccc4c3sc3c(-c5ccccc5)cc5ccccc5c34)cccc12 ; CC1(C)c2ccccc2-c2ccc(N(c3cccc(-c4ccccc4)c3)c3cccc(-c4cccc5c4oc4c(-c6ccccc6)cc6ccccc6c45)c3)cc21]

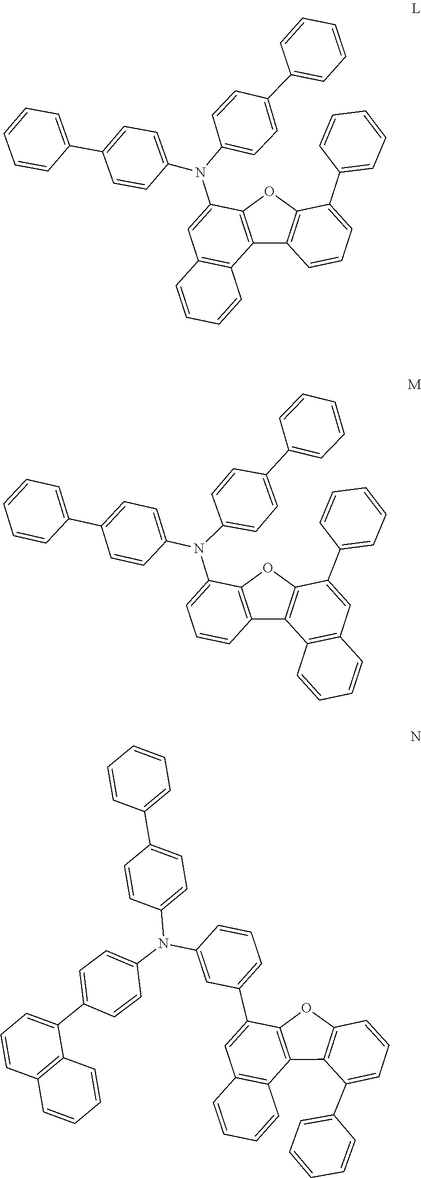 [SMILES: c1ccc(-c2ccc(N(c3ccc(-c4ccccc4)cc3)c3cc4ccccc4c4c3oc3c(-c5ccccc5)cccc34)cc2)cc1 ; c1ccc(-c2ccc(N(c3ccc(-c4ccccc4)cc3)c3cccc4c3oc3c(-c5ccccc5)cc5ccccc5c34)cc2)cc1 ; c1ccc(-c2ccc(N(c3ccc(-c4cccc5ccccc45)cc3)c3cccc(-c4cc5ccccc5c5c4oc4cccc(-c6ccccc6)c45)c3)cc2)cc1]

### Experimental Example 2

### (1) Manufacture of Organic Light Emitting Device

A glass substrate thinly coated with indium tin oxide (ITO) having a thickness of 1,500 Å was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state in order to increase an ITO work function and remove a residual film, and was transferred to a thermal deposition apparatus for organic deposition.

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB. Thereafter, a compound shown in the following Table 10 was deposited to have a thickness of 100 Å as a hole auxiliary layer.

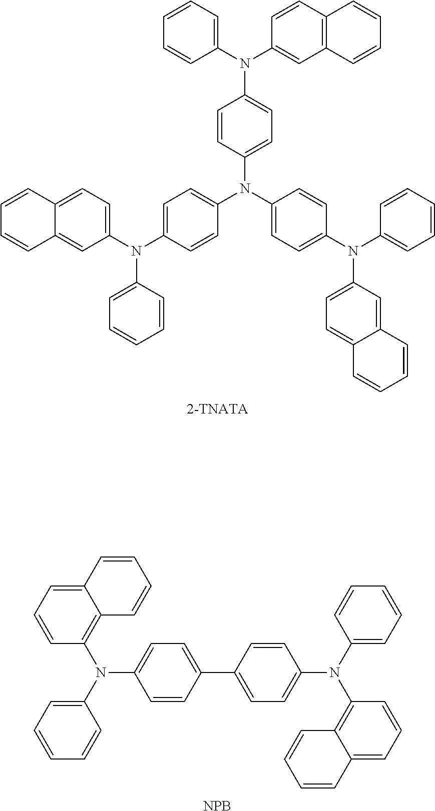 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited by depositing a compound of 9-[4-(4,6-diphenyl-1,3,5-triazin-2-yl)phenyl]-9′-phenyl-3,3′-bi-9H-carbazole as a host to have a thickness of 400 Å and doping the deposited layer with [Ir(ppy)₃] as a green phosphorescent dopant by 7% of the deposited thickness of the light emitting layer. Thereafter, bathocuproine (BCP) was deposited as a hole blocking layer to have a thickness of 60 Å, and Alq₃ was deposited as an electron transport layer to have a thickness of 200 Å thereon. Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1,200 Å on the electron injection layer to form a negative electrode.

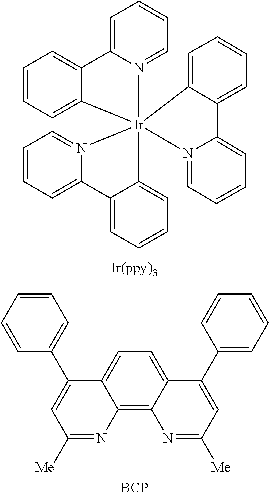

Meanwhile, all the organic compounds required for manufacturing an organic light emitting device were subjected to vacuum sublimed purification under 10⁻⁶ to 10⁻⁸ torr for each material, and used for the manufacture of the organic light emitting device.

### (2) Driving Voltage and Light Emitting Efficiency of Organic Light Emitting Device

For the organic light emitting devices manufactured as above, electroluminescent light emission (EL) characteristics were measured using M7000 manufactured by McScience Inc., and with the measurement results, a service life T₉₅ (unit: h, hour), which was the time when the luminance became 95% compared to the initial luminance when the standard luminance was 6,000 cd/m², was measured using a service life measurement apparatus (M6000) manufactured by McScience Inc.

The characteristics of the organic light emitting devices of the present invention shown with the measurement results are shown in the following Table 10.

TABLE 10

| | | Driving | | Service |
|---|---|---|---|---|
| | | voltage | Efficiency | life |
| | Compound | (V) | (cd/A) | (T₉₅) |
| Comparative | Comparative | 5.79 | 6.5 | 43 |
| Example 12 | Compound A | | | |
| Comparative | Comparative | 6.06 | 6.8 | 42 |
| Example 13 | Compound B | | | |
| Comparative | Comparative | 5.79 | 6.4 | 44 |
| Example 14 | Compound C | | | |
| Comparative | Comparative | 5.76 | 5.9 | 32 |
| Example 15 | Compound D | | | |
| Comparative | Comparative | 5.96 | 4.8 | 43 |
| Example 16 | Compound E | | | |
| Comparative | Comparative | 6.86 | 5.1 | 42 |
| Example 17 | Compound F | | | |
| Comparative | Comparative | 5.95 | 6.0 | 44 |
| Example 18 | Compound G | | | |
| Comparative | Comparative | 5.90 | 4.3 | 32 |
| Example 19 | Compound H | | | |
| Comparative | Comparative | 5.99 | 6.6 | 43 |
| Example 20 | Compound I | | | |
| Comparative | Comparative | 6.74 | 6.2 | 42 |
| Example 21 | Compound J | | | |
| Comparative | Comparative | 6.01 | 5.5 | 48 |
| Example 22 | Compound K | | | |
| Comparative | Comparative | 6.00 | 5.9 | 46 |
| Example 23 | Compound L | | | |
| Comparative | Comparative | 5.98 | 6.4 | 38 |
| Example 24 | Compound M | | | |
| Comparative | Comparative | 5.87 | 6.1 | 49 |
| Example 25 | Compound N | | | |
| Example 66 | Compound 1 | 5.33 | 7.5 | 60 |
| Example 67 | Compound 3 | 5.32 | 8.2 | 56 |
| Example 68 | Compound 5 | 5.24 | 8.0 | 64 |
| Example 69 | Compound 11 | 5.37 | 7.9 | 65 |
| Example 70 | Compound 19 | 5.50 | 7.4 | 68 |
| Example 71 | Compound 20 | 5.46 | 7.3 | 71 |
| Example 72 | Compound 29 | 5.61 | 7.8 | 59 |
| Example 73 | Compound 56 | 5.57 | 7.7 | 55 |
| Example 74 | Compound 70 | 5.30 | 7.9 | 54 |
| Example 75 | Compound 85 | 5.27 | 8.0 | 55 |
| Example 76 | Compound 96 | 5.36 | 7.5 | 61 |
| Example 77 | Compound 101 | 5.34 | 7.9 | 62 |
| Example 78 | Compound 107 | 5.26 | 8.2 | 56 |
| Example 79 | Compound 113 | 5.35 | 8.1 | 69 |
| Example 80 | Compound 121 | 5.41 | 7.6 | 60 |
| Example 81 | Compound 127 | 5.35 | 7.8 | 62 |
| Example 82 | Compound 128 | 5.29 | 7.5 | 56 |
| Example 83 | Compound 129 | 5.44 | 7.6 | 59 |
| Example 84 | Compound 130 | 5.28 | 7.8 | 55 |
| Example 85 | Compound 133 | 5.31 | 7.6 | 51 |
| Example 86 | Compound 136 | 5.30 | 7.7 | 63 |
| Example 87 | Compound 137 | 5.48 | 7.5 | 57 |
| Example 88 | Compound 163 | 5.34 | 7.8 | 61 |
| Example 89 | Compound 189 | 5.26 | 8.0 | 54 |
| Example 90 | Compound 192 | 5.39 | 7.5 | 55 |
| Example 91 | Compound 193 | 5.40 | 7.6 | 62 |
| Example 92 | Compound 232 | 5.22 | 7.9 | 56 |
| Example 93 | Compound 236 | 5.24 | 7.9 | 59 |
| Example 94 | Compound 238 | 5.30 | 8.2 | 57 |
| Example 95 | Compound 240 | 5.29 | 7.8 | 63 |
| Example 96 | Compound 241 | 5.32 | 7.5 | 57 |
| Example 97 | Compound 250 | 5.33 | 7.6 | 62 |
| Example 98 | Compound 252 | 5.35 | 7.7 | 50 |
| Example 99 | Compound 256 | 5.29 | 7.9 | 52 |
| Example 100 | Compound 277 | 5.44 | 8.0 | 63 |
| Example 101 | Compound 281 | 5.30 | 7.9 | 55 |
| Example 102 | Compound 292 | 5.47 | 7.9 | 61 |
| Example 103 | Compound 317 | 5.51 | 8.1 | 54 |
| Example 104 | Compound 352 | 5.23 | 7.6 | 53 |
| Example 105 | Compound 363 | 5.37 | 7.7 | 61 |
| Example 106 | Compound 376 | 5.29 | 7.5 | 64 |
| Example 107 | Compound 377 | 5.30 | 7.5 | 52 |
| Example 108 | Compound 401 | 5.48 | 7.6 | 60 |
| Example 109 | Compound 443 | 5.31 | 7.9 | 61 |
| Example 110 | Compound 451 | 5.23 | 7.6 | 63 |
| Example 111 | Compound 454 | 5.37 | 7.7 | 51 |
| Example 112 | Compound 456 | 5.29 | 7.5 | 54 |
| Example 113 | Compound 459 | 5.30 | 7.5 | 52 |
| Example 114 | Compound 482 | 5.48 | 7.6 | 53 |
| Example 115 | Compound 485 | 5.35 | 8.1 | 59 |
| Example 116 | Compound 489 | 5.41 | 7.6 | 56 |
| Example 117 | Compound 490 | 5.40 | 7.9 | 60 |
| Example 118 | Compound 501 | 5.27 | 7.8 | 55 |
| Example 119 | Compound 502 | 5.33 | 7.8 | 54 |
| Example 120 | Compound 512 | 5.24 | 8.0 | 57 |
| Example 121 | Compound 522 | 5.37 | 7.9 | 51 |
| Example 122 | Compound 525 | 5.50 | 7.4 | 56 |
| Example 123 | Compound 528 | 5.46 | 7.3 | 61 |
| Example 124 | Compound 539 | 5.61 | 7.8 | 59 |
| Example 125 | Compound 548 | 5.33 | 7.6 | 62 |
| Example 126 | Compound 549 | 5.35 | 7.7 | 60 |
| Example 127 | Compound 550 | 5.38 | 7.8 | 58 |
| Example 128 | Compound 570 | 5.47 | 8.1 | 59 |
| Example 129 | Compound 577 | 5.43 | 7.9 | 56 |
| Example 130 | Compound 582 | 5.29 | 7.5 | 61 |
| Example 131 | Compound 583 | 5.30 | 7.5 | 59 |
| Example 132 | Compound 591 | 5.48 | 7.6 | 62 |
| Example 133 | Compound 592 | 5.31 | 7.9 | 60 |
| Example 134 | Compound 595 | 5.33 | 7.8 | 56 |
| Example 135 | Compound 607 | 5.39 | 7.7 | 60 |
| Example 136 | Compound 622 | 5.29 | 7.9 | 55 |
| Example 137 | Compound 631 | 5.44 | 8.0 | 53 |
| Example 138 | Compound 638 | 5.30 | 7.9 | 55 |

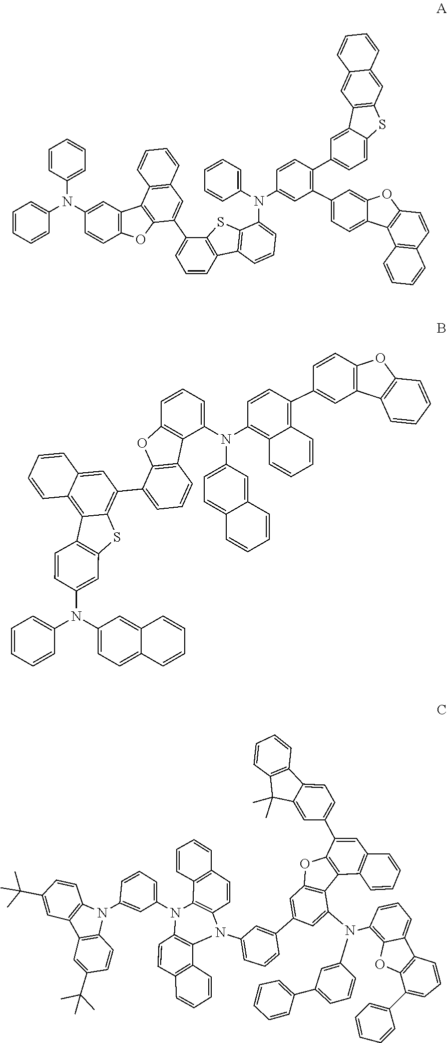

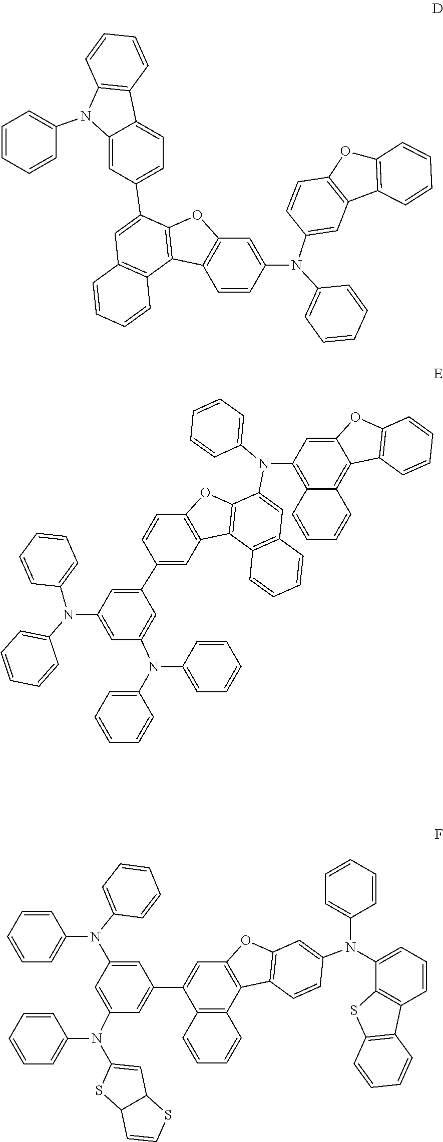 [SMILES: c1ccc(N(c2ccc3c(c2)oc2c(-c4ccc5c6ccccc6n(-c6ccccc6)c5c4)cc4ccccc4c23)c2ccc3oc4ccccc4c3c2)cc1 ; c1ccc(N(c2ccccc2)c2cc(-c3ccc4oc5c(N(c6ccccc6)c6cc7oc8ccccc8c7c7ccccc67)cc6ccccc6c5c4c3)cc(N(c3ccccc3)c3ccccc3)c2)cc1 ; C1=CC2SC(N(c3ccccc3)c3cc(-c4cc5oc6cc(N(c7ccccc7)c7cccc8c7sc7ccccc78)ccc6c5c5ccccc45)cc(N(c4ccccc4)c4ccccc4)c3)=CC2S1]

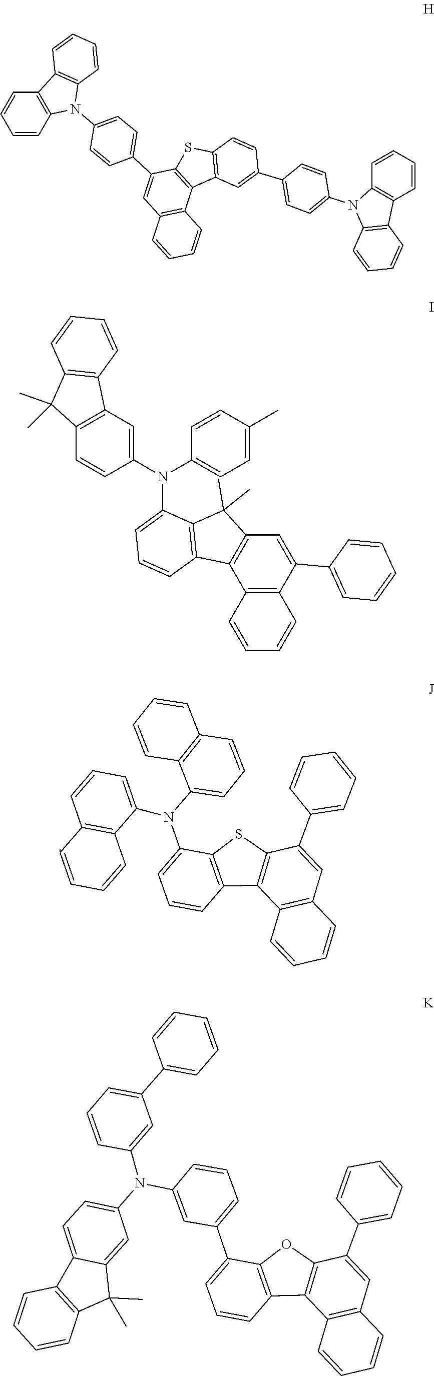 [SMILES: c1ccc2c(c1)cc(-c1ccc(-n3c4ccccc4c4ccccc43)cc1)c1sc3ccc(-c4ccc(-n5c6ccccc6c6ccccc65)cc4)cc3c12 ; Cc1ccc(N(c2ccc3c(c2)-c2ccccc2C3(C)C)c2cccc3c2C(C)(C)c2cc(-c4ccccc4)c4ccccc4c2-3)cc1 ; Cc1cccc2c(N(c3cccc4ccccc34)c3cccc4c3sc3c(-c5ccccc5)cc5ccccc5c34)cccc12 ; CC1(C)c2ccccc2-c2ccc(N(c3cccc(-c4ccccc4)c3)c3cccc(-c4cccc5c4oc4c(-c6ccccc6)cc6ccccc6c45)c3)cc21]

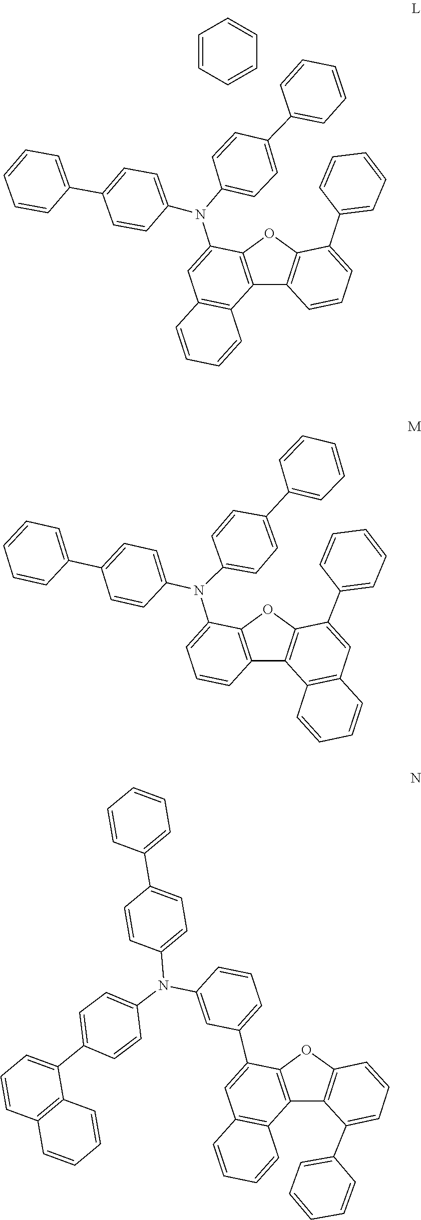

When the compound according to the present application is used for an organic light emitting device, the driving voltage of the device could be lowered, the light efficiency of the device could be improved, and the service life characteristics of the device could be improved due to the thermal stability of the compound.

Specifically, when NPB, which is the hole transport layer material of Comparative Example 1, was compared with the compound represented by Chemical Formula 1 of the present invention, which is the hole transport layer material of Examples 1 to 73, it could be confirmed that the structures of some substituents are similar, but the substitution positions or types of the substituents are different.

Since Comparative Compounds A to C have a high molecular weight, the thermal stability is poor, which makes the molecules unstable during deposition, resulting in high driving voltage and shortened service life.

Furthermore, since Comparative Compounds D to G have a structure where arylamine is di-substituted, the hole mobility is much faster than in a structure where arylamine is mono-substituted, so that the charge balance between holes and electrons in the light emitting layer is not achieved, preventing efficient exciton formation. This increases the driving voltage of the device and decreases the efficiency of the device.

In the case of Comparative Compounds H to N, the positions substituted in the present invention have a relatively faster hole mobility than the positions substituted in the comparative compounds, an appropriate energy level and an appropriate band gap are formed to increase the number of excitons in the light emitting region. Increasing excitons in the light emitting region means having an effect of reducing the driving voltage of the device and an effect of increasing efficiency.

Therefore, taking the above contents into consideration, when the heterocyclic compound of Chemical Formula 1 is used as a material for the hole transport layer in the organic light emitting device, an appropriate energy level and an appropriate band gap are formed to increase the number of excitons in the light emitting region. Increasing excitons in the light emitting region means having an effect of reducing the driving voltage of the device and an effect of increasing efficiency.

1. A heterocyclic compound represented by the following Chemical Formula 1: 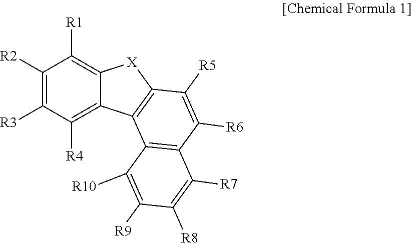
wherein, in Chemical Formula 1,
X is O; or S,
R1 to R10 are the same as or different from each other, and are each independently represented by hydrogen; deuterium; the following Chemical Formula 2; or following Chemical Formula 3,
one of R3 and R4; and R5 are represented by the following Chemical Formula 2 or the following Chemical Formula 3, 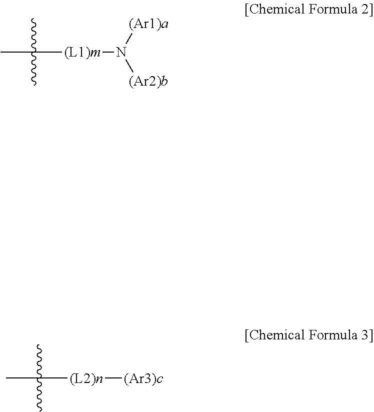
in Chemical Formulae 2 and 3,
L1 and L2 are the same as or different from each other, and are each independently a direct bond; a substituted or unsubstituted C6 to C60 arylene group; or a substituted or unsubstituted C2 to C60 heteroarylene group,
m and n are an integer from 0 to 4,
Ar1 to Ar3 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a, b, and c are an integer from 1 to 4, and
when m, n, a, b, and c are 2 or higher, substituents in the parenthesis are the same as or different from each other.

2. The heterocyclic compound of claim 1, wherein Chemical Formula 1 is represented by any one of the following Chemical Formulae 4 to 7: 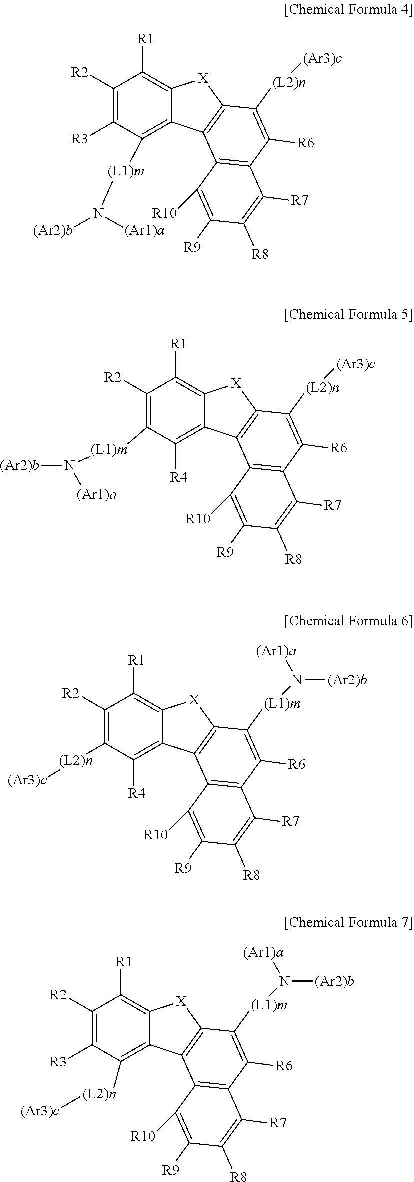
in Chemical Formulae 4 to 7,
the definitions of L1, L2, m, n, Ar1 to Ar3, X, a, b, and c are the same as the definitions in Chemical Formula 1, and
R1 to R4 and R6 to R10 are the same as or different from each other, and are each independently hydrogen; or deuterium.

3. The heterocyclic compound of claim 1, wherein Chemical Formula 2 is represented by any one of the following Chemical Formulae 2-1 to 2-3: 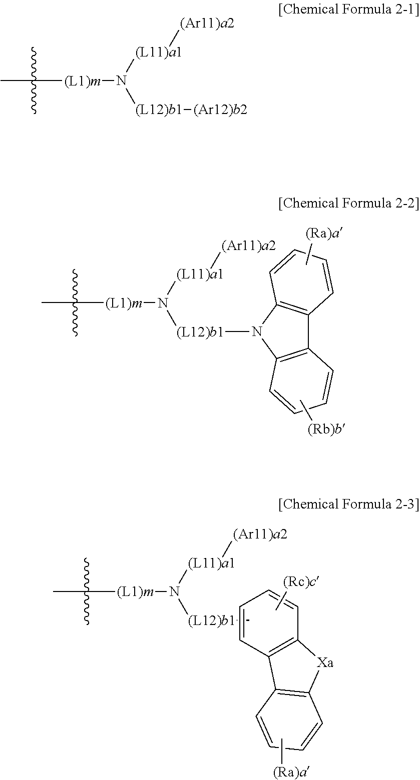
in Chemical Formulae 2-1 to 2-3,
the definitions of L1 and m are the same as the definitions in Chemical Formula 2,
L11 and L12 are the same as or different from each other, and are each independently a direct bond; a substituted or unsubstituted C6 to C60 arylene group; or a substituted or unsubstituted C2 to C60 heteroarylene group,
a1 and bi are the same as or different from each other, and are each independently an integer from 0 to 3,
a2 and b2 are the same as or different from each other, and are each independently an integer from 1 to 3,
Ar1 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
Ar12 is a substituted or unsubstituted C6 to C60 aryl group,
Xa is O; S; or NRc,
Ra, Rb, and Rc are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a′ and b′ are an integer from 0 to 4,
c′ is an integer from 0 to 3, and
when a′, b′, c′, a1, a2, b2, and b1 are 2 or higher, substituents in the parenthesis are the same as or different from each other.

4. The heterocyclic compound of claim 1, wherein a deuterium content of the heterocyclic compound of Chemical Formula 1 is 0%, or 3% to 100%.

5. The heterocyclic compound of claim 1, wherein Chemical Formula 1 is represented by any one of the following compounds:

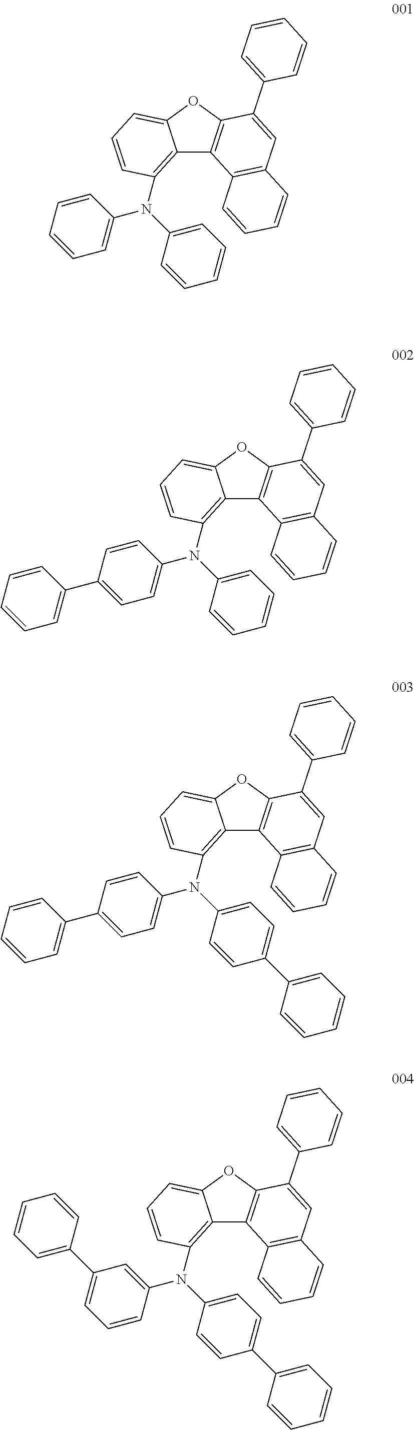 [SMILES: c1ccc(-c2cc3ccccc3c3c2oc2cccc(N(c4ccccc4)c4ccccc4)c23)cc1 ; c1ccc(-c2ccc(N(c3ccccc3)c3cccc4oc5c(-c6ccccc6)cc6ccccc6c5c34)cc2)cc1 ; c1ccc(-c2ccc(N(c3ccc(-c4ccccc4)cc3)c3cccc4oc5c(-c6ccccc6)cc6ccccc6c5c34)cc2)cc1 ; c1ccc(-c2ccc(N(c3cccc(-c4ccccc4)c3)c3cccc4oc5c(-c6ccccc6)cc6ccccc6c5c34)cc2)cc1]

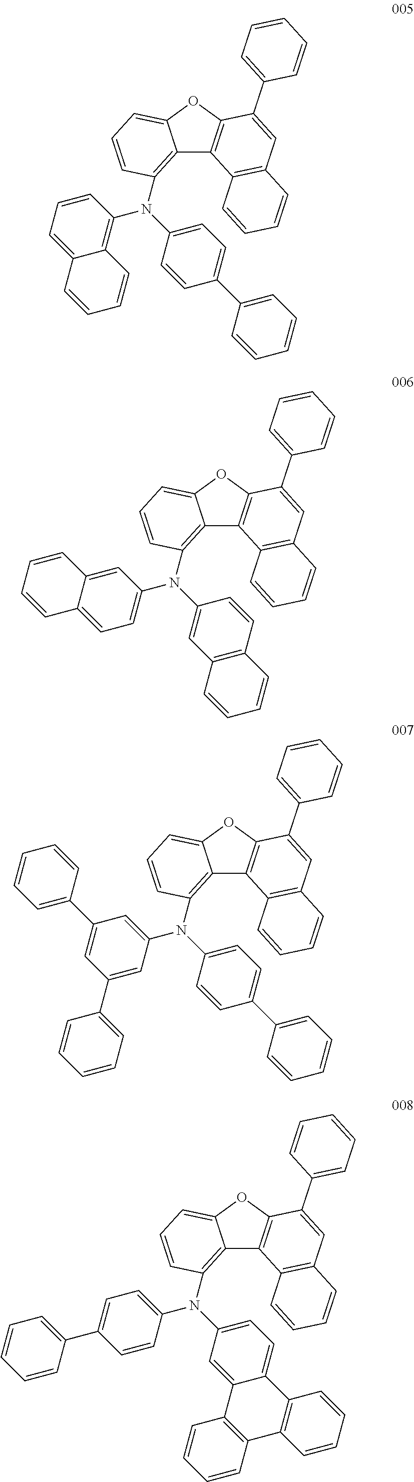

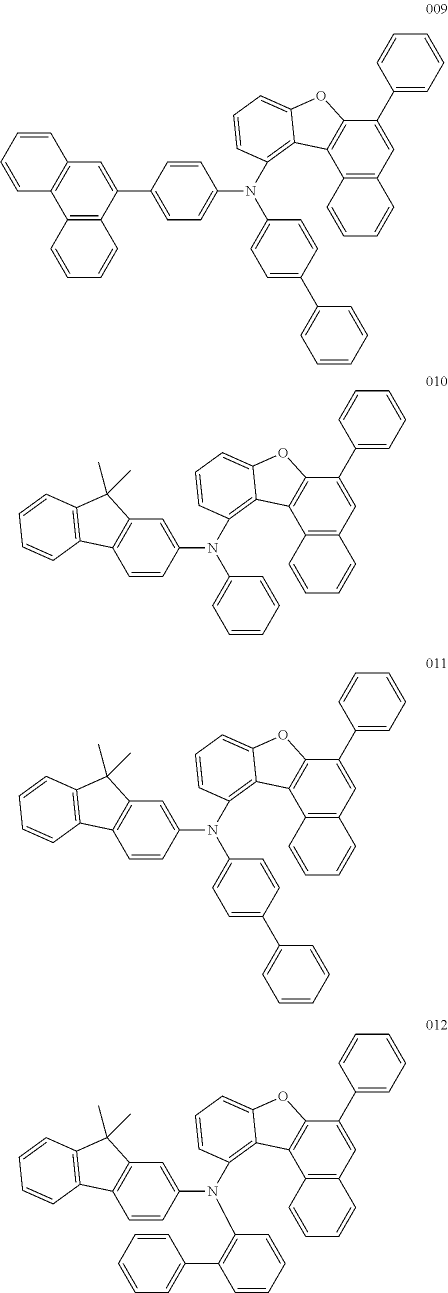

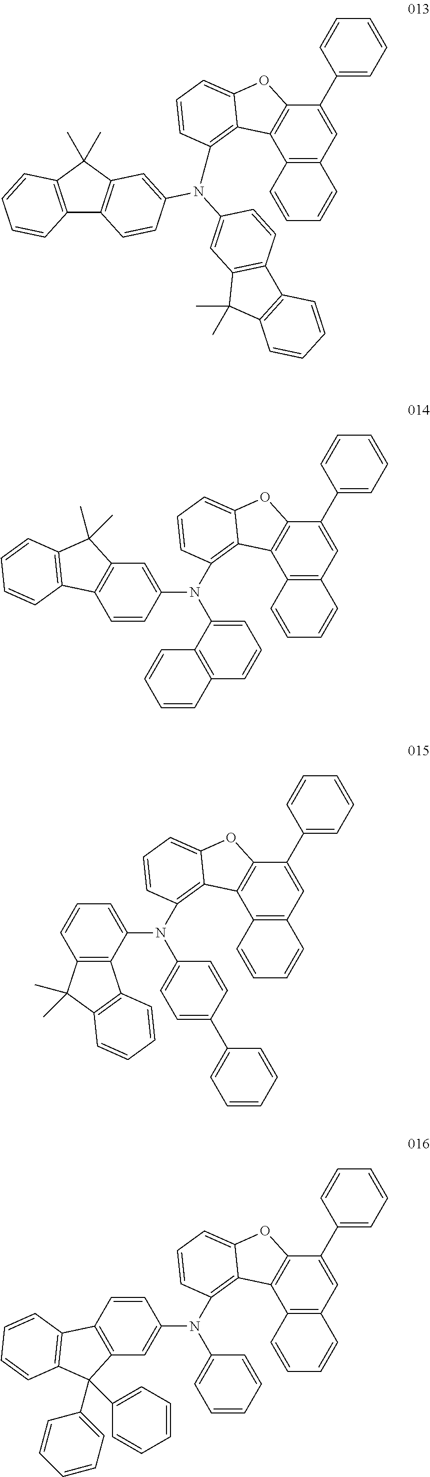 [SMILES: CC1(C)c2ccccc2-c2ccc(N(c3ccc4c(c3)C(C)(C)c3ccccc3-4)c3cccc4oc5c(-c6ccccc6)cc6ccccc6c5c34)cc21 ; CC1(C)c2ccccc2-c2ccc(N(c3cccc4ccccc34)c3cccc4oc5c(-c6ccccc6)cc6ccccc6c5c34)cc21 ; CC1(C)c2ccccc2-c2c(N(c3ccc(-c4ccccc4)cc3)c3cccc4oc5c(-c6ccccc6)cc6ccccc6c5c34)cccc21 ; c1ccc(-c2cc3ccccc3c3c2oc2cccc(N(c4ccccc4)c4ccc5c(c4)C(c4ccccc4)(c4ccccc4)c4ccccc4-5)c23)cc1]

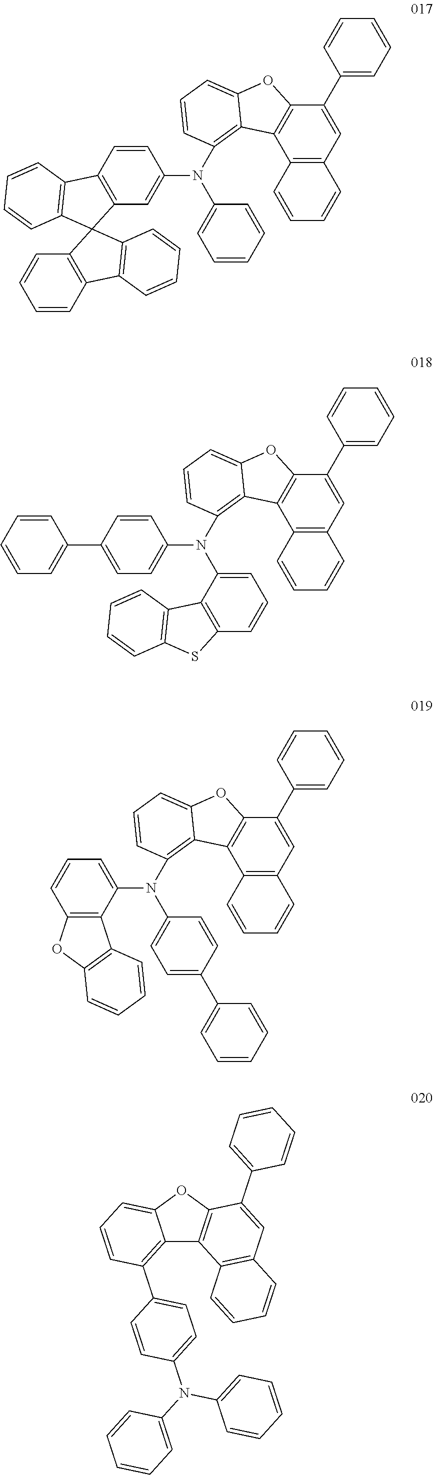 [SMILES: c1ccc(-c2cc3ccccc3c3c2oc2cccc(N(c4ccccc4)c4ccc5c(c4)C4(c6ccccc6-c6ccccc64)c4ccccc4-5)c23)cc1 ; c1ccc(-c2ccc(N(c3cccc4sc5ccccc5c34)c3cccc4oc5c(-c6ccccc6)cc6ccccc6c5c34)cc2)cc1 ; c1ccc(-c2ccc(N(c3cccc4oc5ccccc5c34)c3cccc4oc5c(-c6ccccc6)cc6ccccc6c5c34)cc2)cc1 ; c1ccc(-c2cc3ccccc3c3c2oc2cccc(-c4ccc(N(c5ccccc5)c5ccccc5)cc4)c23)cc1]

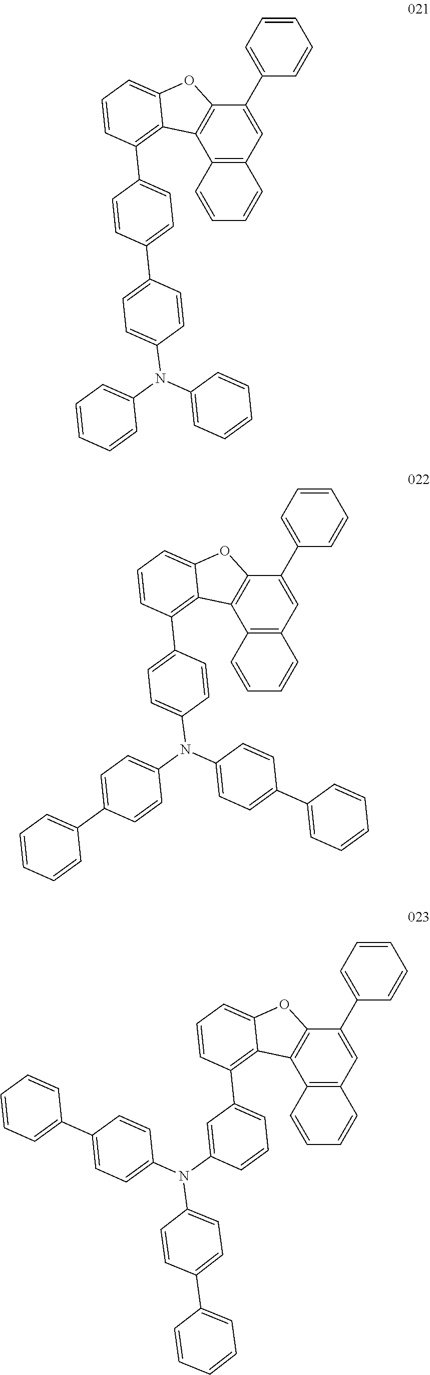 [SMILES: c1ccc(-c2cc3ccccc3c3c2oc2cccc(-c4ccc(-c5ccc(N(c6ccccc6)c6ccccc6)cc5)cc4)c23)cc1 ; c1ccc(-c2ccc(N(c3ccc(-c4ccccc4)cc3)c3ccc(-c4cccc5oc6c(-c7ccccc7)cc7ccccc7c6c45)cc3)cc2)cc1 ; c1ccc(-c2ccc(N(c3ccc(-c4ccccc4)cc3)c3cccc(-c4cccc5oc6c(-c7ccccc7)cc7ccccc7c6c45)c3)cc2)cc1]

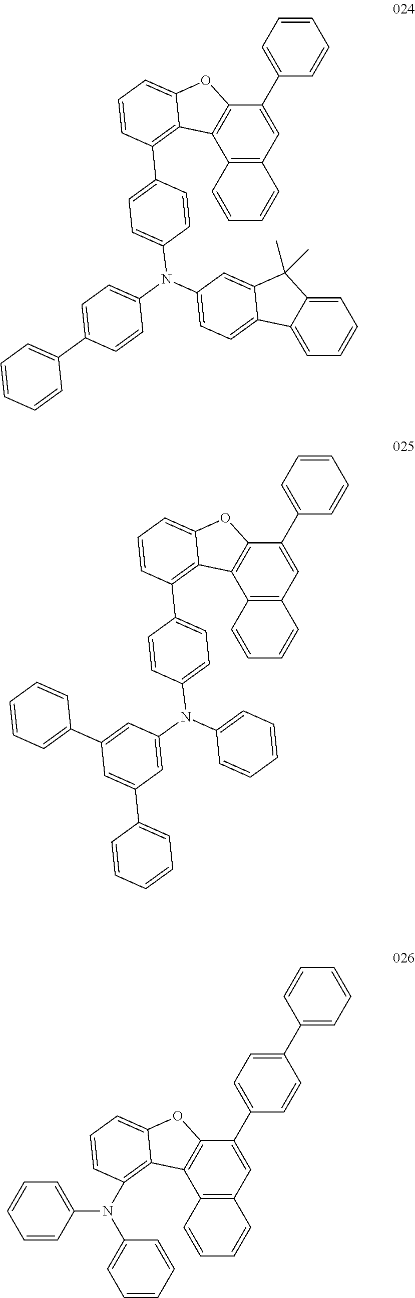 [SMILES: CC1(C)c2ccccc2-c2ccc(N(c3ccc(-c4ccccc4)cc3)c3ccc(-c4cccc5oc6c(-c7ccccc7)cc7ccccc7c6c45)cc3)cc21 ; c1ccc(-c2cc(-c3ccccc3)cc(N(c3ccccc3)c3ccc(-c4cccc5oc6c(-c7ccccc7)cc7ccccc7c6c45)cc3)c2)cc1 ; c1ccc(-c2ccc(-c3cc4ccccc4c4c3oc3cccc(N(c5ccccc5)c5ccccc5)c34)cc2)cc1]

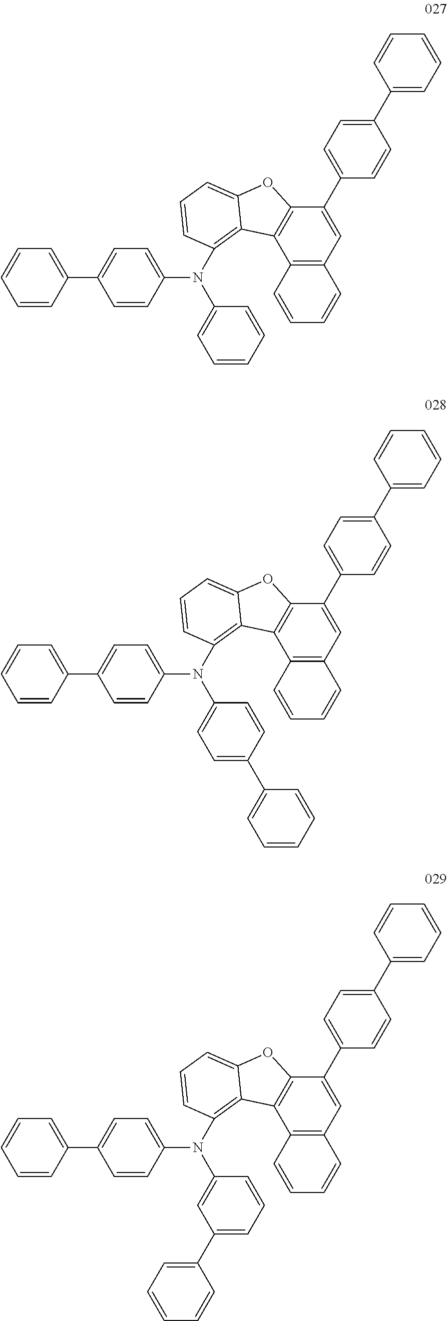 [SMILES: c1ccc(-c2ccc(-c3cc4ccccc4c4c3oc3cccc(N(c5ccccc5)c5ccc(-c6ccccc6)cc5)c34)cc2)cc1 ; c1ccc(-c2ccc(-c3cc4ccccc4c4c3oc3cccc(N(c5ccc(-c6ccccc6)cc5)c5ccc(-c6ccccc6)cc5)c34)cc2)cc1 ; c1ccc(-c2ccc(-c3cc4ccccc4c4c3oc3cccc(N(c5ccc(-c6ccccc6)cc5)c5cccc(-c6ccccc6)c5)c34)cc2)cc1]

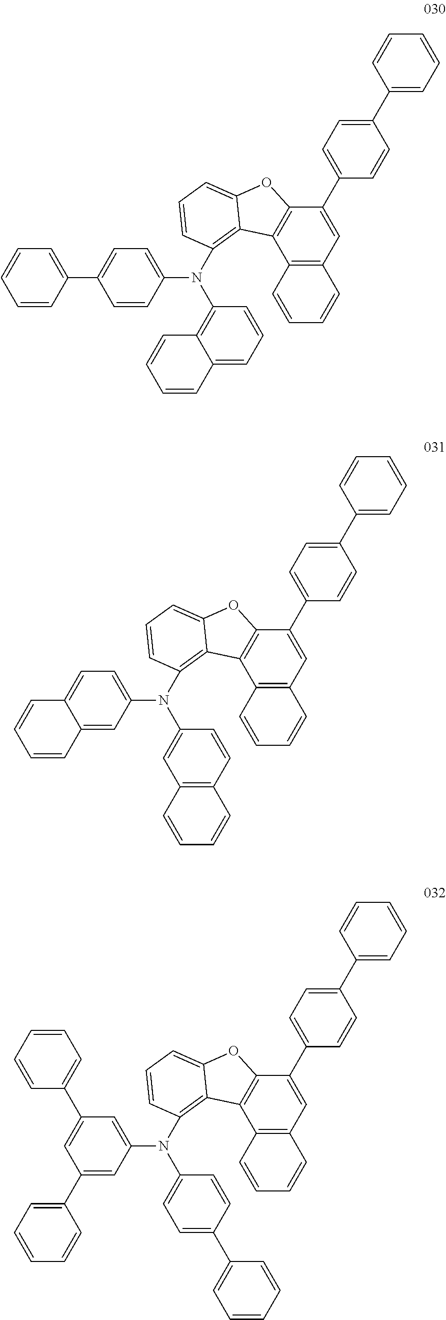 [SMILES: c1ccc(-c2ccc(-c3cc4ccccc4c4c3oc3cccc(N(c5ccc(-c6ccccc6)cc5)c5cccc6ccccc56)c34)cc2)cc1 ; c1ccc(-c2ccc(-c3cc4ccccc4c4c3oc3cccc(N(c5ccc6ccccc6c5)c5ccc6ccccc6c5)c34)cc2)cc1 ; c1ccc(-c2ccc(-c3cc4ccccc4c4c3oc3cccc(N(c5ccc(-c6ccccc6)cc5)c5cc(-c6ccccc6)cc(-c6ccccc6)c5)c34)cc2)cc1]

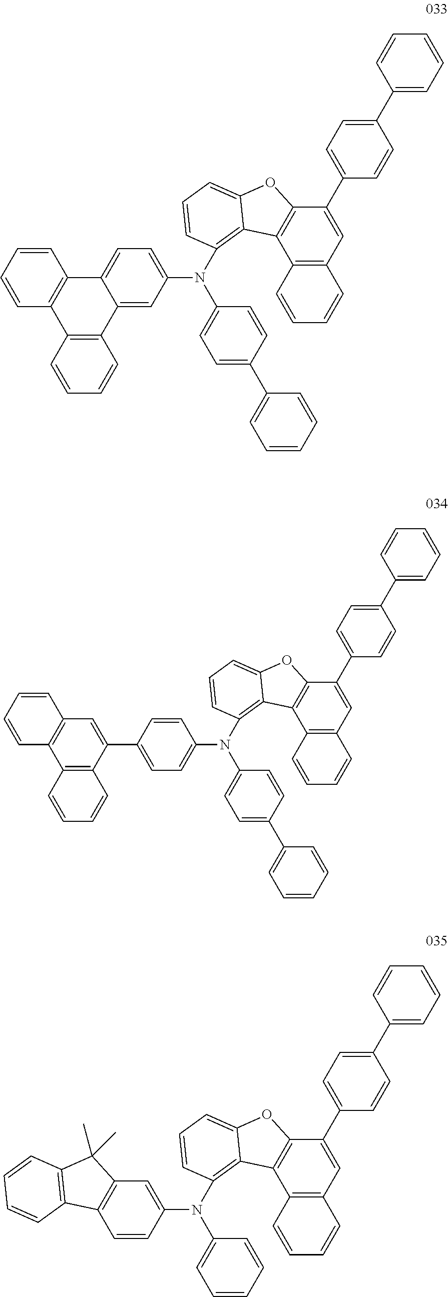 [SMILES: c1ccc(-c2ccc(-c3cc4ccccc4c4c3oc3cccc(N(c5ccc(-c6ccccc6)cc5)c5ccc6c7ccccc7c7ccccc7c6c5)c34)cc2)cc1 ; c1ccc(-c2ccc(-c3cc4ccccc4c4c3oc3cccc(N(c5ccc(-c6ccccc6)cc5)c5ccc(-c6cc7ccccc7c7ccccc67)cc5)c34)cc2)cc1 ; CC1(C)c2ccccc2-c2ccc(N(c3ccccc3)c3cccc4oc5c(-c6ccc(-c7ccccc7)cc6)cc6ccccc6c5c34)cc21]

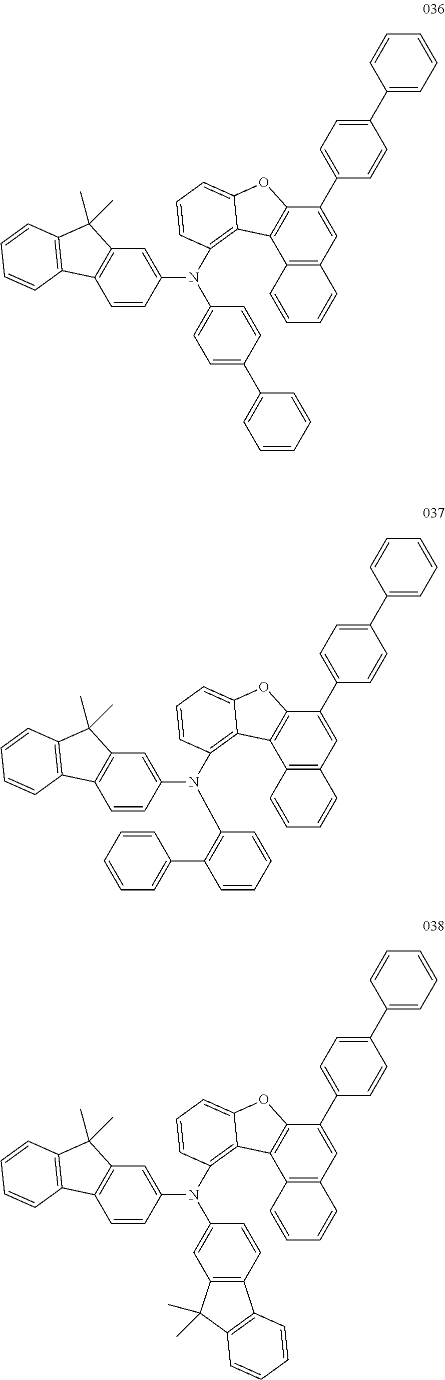 [SMILES: CC1(C)c2ccccc2-c2ccc(N(c3ccc(-c4ccccc4)cc3)c3cccc4oc5c(-c6ccc(-c7ccccc7)cc6)cc6ccccc6c5c34)cc21 ; CC1(C)c2ccccc2-c2ccc(N(c3ccccc3-c3ccccc3)c3cccc4oc5c(-c6ccc(-c7ccccc7)cc6)cc6ccccc6c5c34)cc21 ; CC1(C)c2ccccc2-c2ccc(N(c3ccc4c(c3)C(C)(C)c3ccccc3-4)c3cccc4oc5c(-c6ccc(-c7ccccc7)cc6)cc6ccccc6c5c34)cc21]

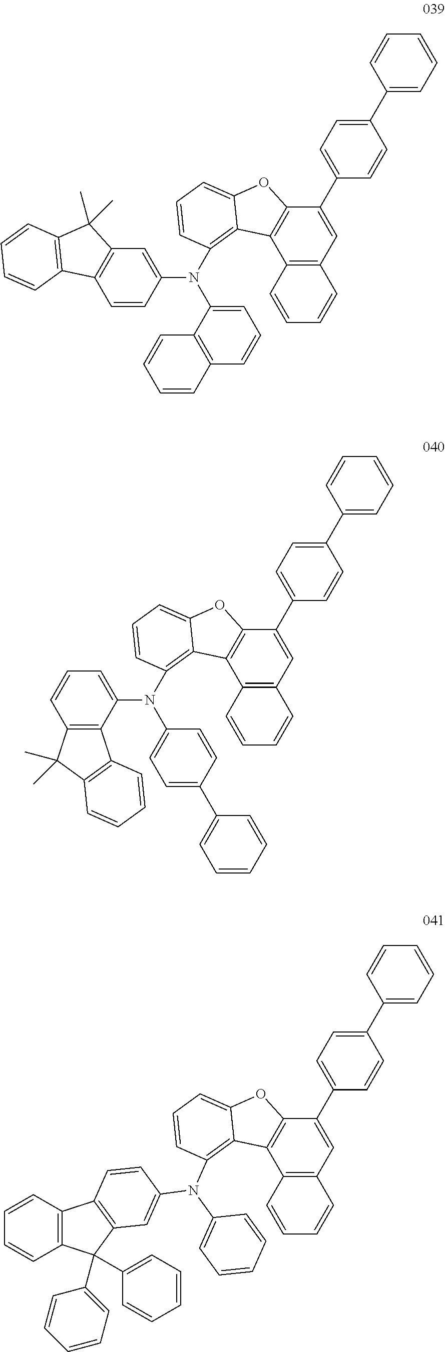 [SMILES: CC1(C)c2ccccc2-c2ccc(N(c3cccc4ccccc34)c3cccc4oc5c(-c6ccc(-c7ccccc7)cc6)cc6ccccc6c5c34)cc21 ; CC1(C)c2ccccc2-c2c(N(c3ccc(-c4ccccc4)cc3)c3cccc4oc5c(-c6ccc(-c7ccccc7)cc6)cc6ccccc6c5c34)cccc21 ; c1ccc(-c2ccc(-c3cc4ccccc4c4c3oc3cccc(N(c5ccccc5)c5ccc6c(c5)C(c5ccccc5)(c5ccccc5)c5ccccc5-6)c34)cc2)cc1]

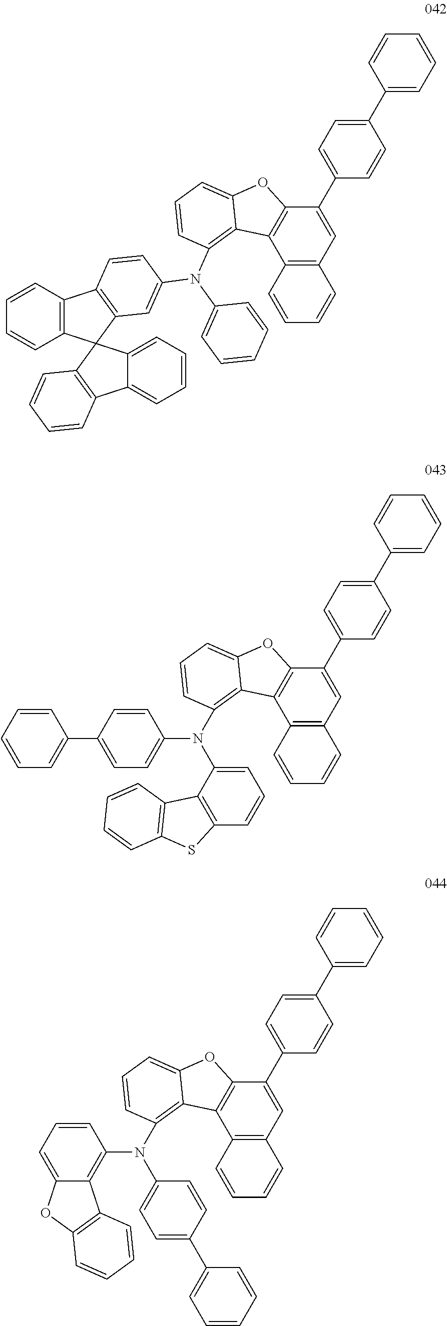 [SMILES: c1ccc(-c2ccc(-c3cc4ccccc4c4c3oc3cccc(N(c5ccccc5)c5ccc6c(c5)C5(c7ccccc7-c7ccccc75)c5ccccc5-6)c34)cc2)cc1 ; c1ccc(-c2ccc(-c3cc4ccccc4c4c3oc3cccc(N(c5ccc(-c6ccccc6)cc5)c5cccc6sc7ccccc7c56)c34)cc2)cc1 ; c1ccc(-c2ccc(-c3cc4ccccc4c4c3oc3cccc(N(c5ccc(-c6ccccc6)cc5)c5cccc6oc7ccccc7c56)c34)cc2)cc1]

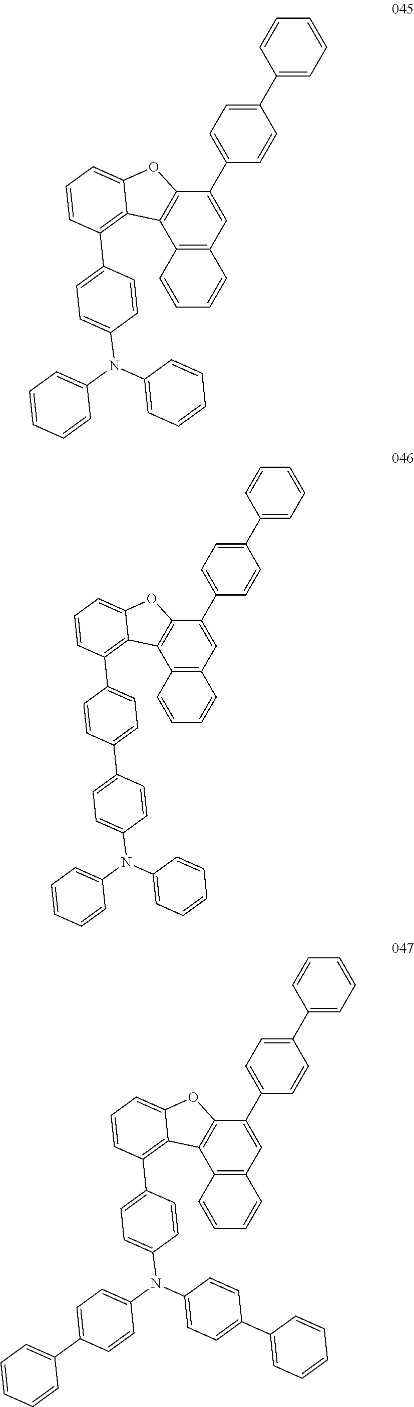 [SMILES: c1ccc(-c2ccc(-c3cc4ccccc4c4c3oc3cccc(-c5ccc(N(c6ccccc6)c6ccccc6)cc5)c34)cc2)cc1 ; c1ccc(-c2ccc(-c3cc4ccccc4c4c3oc3cccc(-c5ccc(-c6ccc(N(c7ccccc7)c7ccccc7)cc6)cc5)c34)cc2)cc1 ; c1ccc(-c2ccc(-c3cc4ccccc4c4c3oc3cccc(-c5ccc(N(c6ccc(-c7ccccc7)cc6)c6ccc(-c7ccccc7)cc6)cc5)c34)cc2)cc1]

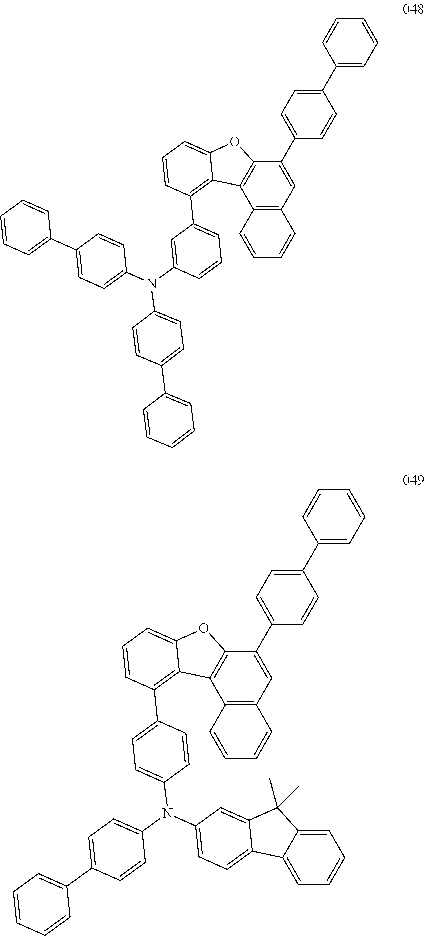 [SMILES: c1ccc(-c2ccc(-c3cc4ccccc4c4c3oc3cccc(-c5cccc(N(c6ccc(-c7ccccc7)cc6)c6ccc(-c7ccccc7)cc6)c5)c34)cc2)cc1 ; CC1(C)c2ccccc2-c2ccc(N(c3ccc(-c4ccccc4)cc3)c3ccc(-c4cccc5oc6c(-c7ccc(-c8ccccc8)cc7)cc7ccccc7c6c45)cc3)cc21]

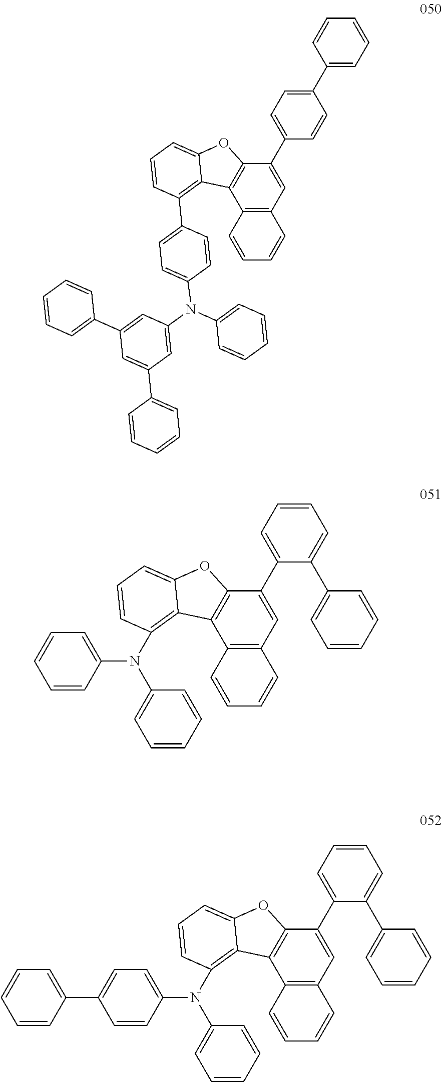 [SMILES: c1ccc(-c2ccc(-c3cc4ccccc4c4c3oc3cccc(-c5ccc(N(c6ccccc6)c6cc(-c7ccccc7)cc(-c7ccccc7)c6)cc5)c34)cc2)cc1 ; c1ccc(-c2ccccc2-c2cc3ccccc3c3c2oc2cccc(N(c4ccccc4)c4ccccc4)c23)cc1 ; c1ccc(-c2ccc(N(c3ccccc3)c3cccc4oc5c(-c6ccccc6-c6ccccc6)cc6ccccc6c5c34)cc2)cc1]

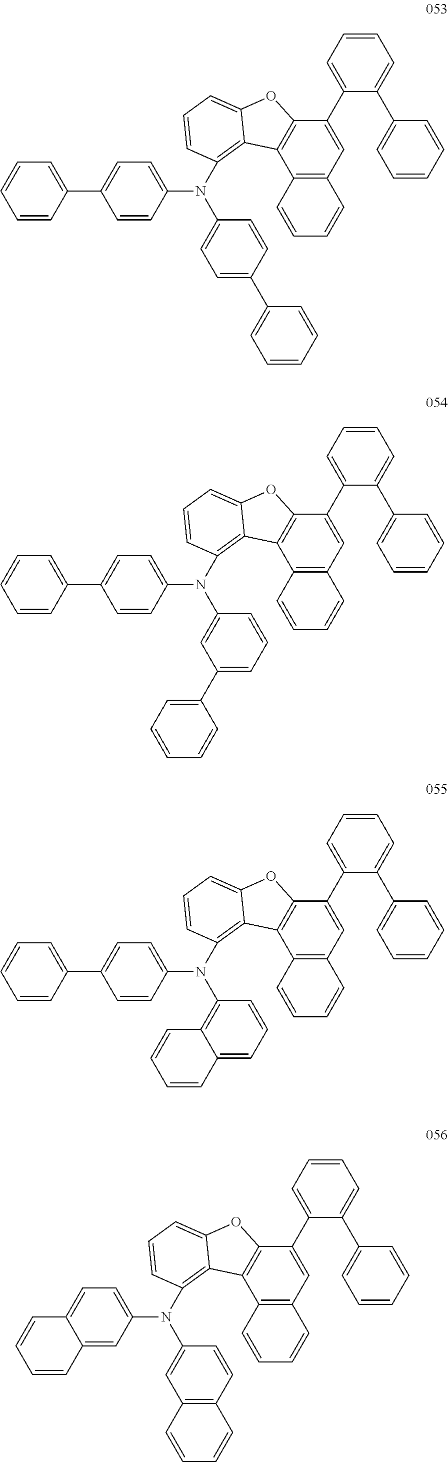 [SMILES: c1ccc(-c2ccc(N(c3ccc(-c4ccccc4)cc3)c3cccc4oc5c(-c6ccccc6-c6ccccc6)cc6ccccc6c5c34)cc2)cc1 ; c1ccc(-c2ccc(N(c3cccc(-c4ccccc4)c3)c3cccc4oc5c(-c6ccccc6-c6ccccc6)cc6ccccc6c5c34)cc2)cc1 ; c1ccc(-c2ccc(N(c3cccc4ccccc34)c3cccc4oc5c(-c6ccccc6-c6ccccc6)cc6ccccc6c5c34)cc2)cc1 ; c1ccc(-c2ccccc2-c2cc3ccccc3c3c2oc2cccc(N(c4ccc5ccccc5c4)c4ccc5ccccc5c4)c23)cc1]

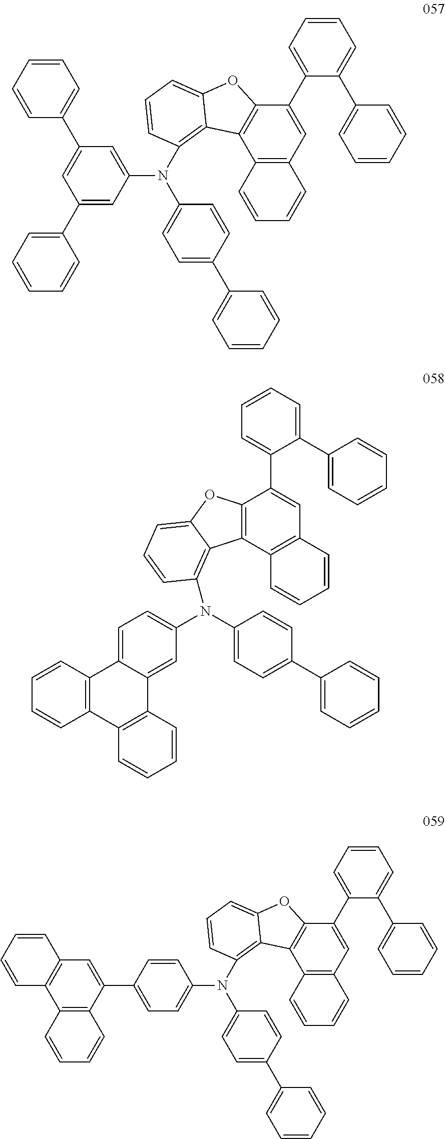

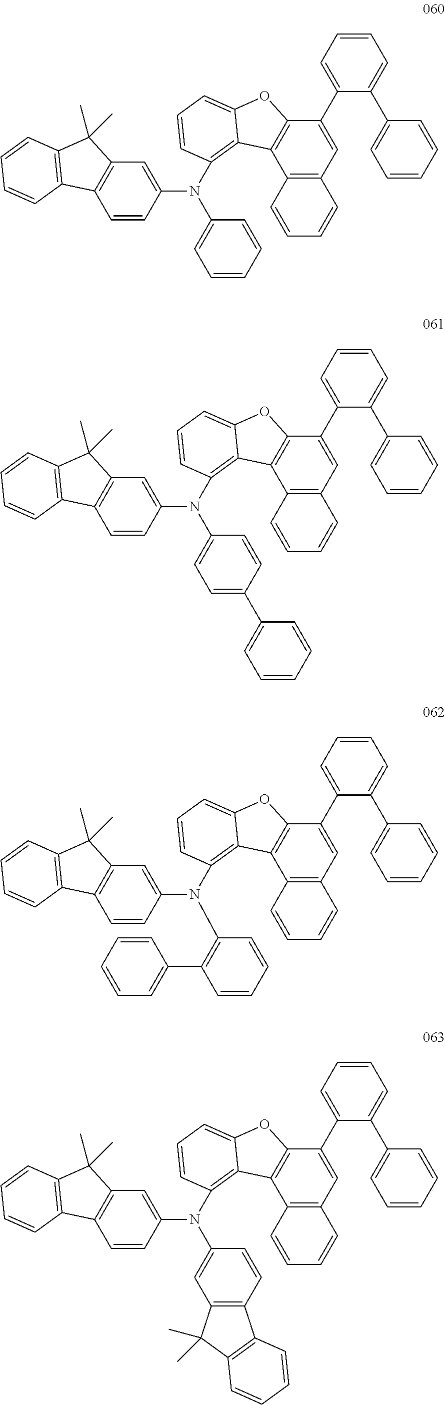 [SMILES: CC1(C)c2ccccc2-c2ccc(N(c3ccccc3)c3cccc4oc5c(-c6ccccc6-c6ccccc6)cc6ccccc6c5c34)cc21 ; CC1(C)c2ccccc2-c2ccc(N(c3ccc(-c4ccccc4)cc3)c3cccc4oc5c(-c6ccccc6-c6ccccc6)cc6ccccc6c5c34)cc21 ; CC1(C)c2ccccc2-c2ccc(N(c3ccccc3-c3ccccc3)c3cccc4oc5c(-c6ccccc6-c6ccccc6)cc6ccccc6c5c34)cc21 ; CC1(C)c2ccccc2-c2ccc(N(c3ccc4c(c3)C(C)(C)c3ccccc3-4)c3cccc4oc5c(-c6ccccc6-c6ccccc6)cc6ccccc6c5c34)cc21]

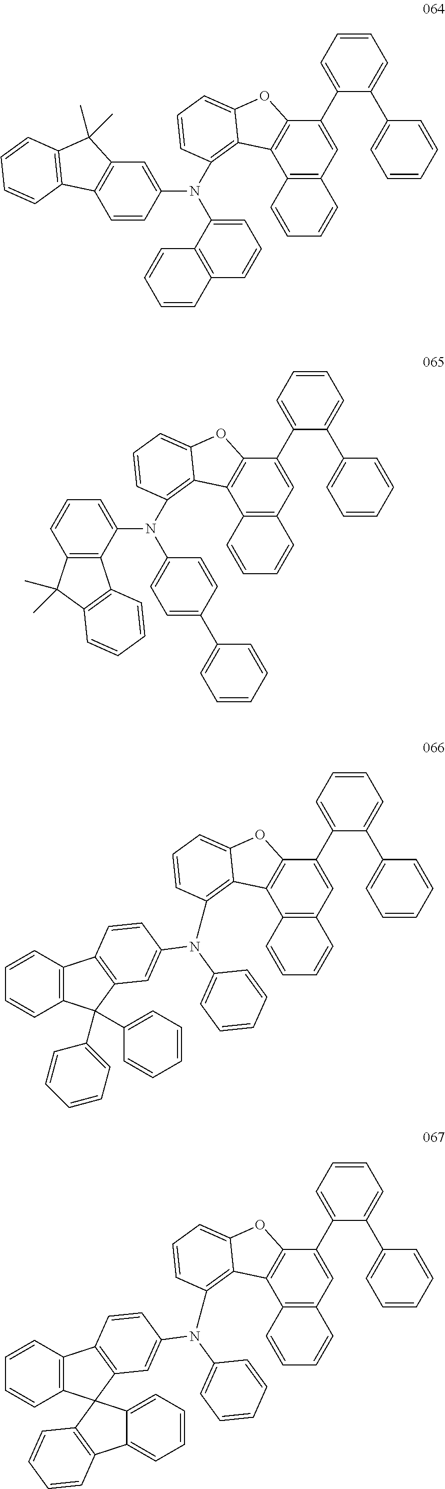 [SMILES: CC1(C)c2ccccc2-c2ccc(N(c3cccc4ccccc34)c3cccc4oc5c(-c6ccccc6-c6ccccc6)cc6ccccc6c5c34)cc21 ; CC1(C)c2ccccc2-c2c(N(c3ccc(-c4ccccc4)cc3)c3cccc4oc5c(-c6ccccc6-c6ccccc6)cc6ccccc6c5c34)cccc21 ; c1ccc(-c2ccccc2-c2cc3ccccc3c3c2oc2cccc(N(c4ccccc4)c4ccc5c(c4)C(c4ccccc4)(c4ccccc4)c4ccccc4-5)c23)cc1 ; c1ccc(-c2ccccc2-c2cc3ccccc3c3c2oc2cccc(N(c4ccccc4)c4ccc5c(c4)C4(c6ccccc6-c6ccccc64)c4ccccc4-5)c23)cc1]

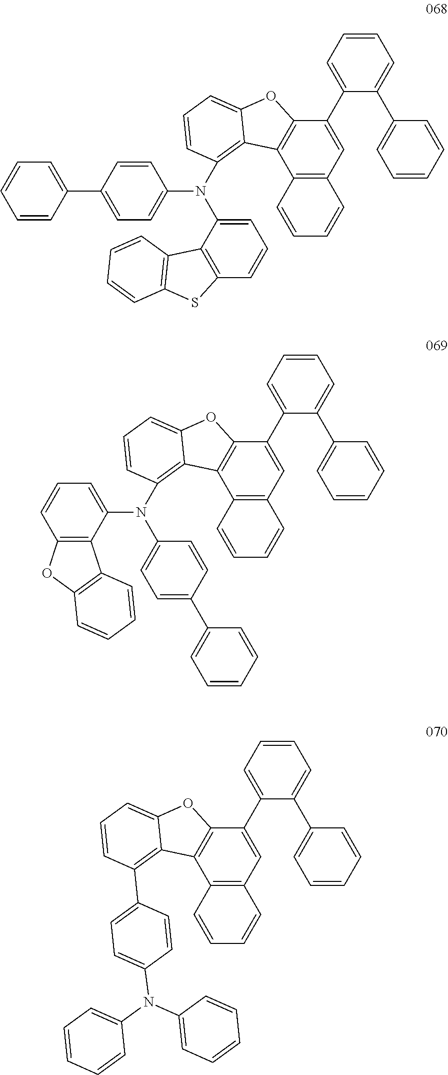 [SMILES: c1ccc(-c2ccc(N(c3cccc4sc5ccccc5c34)c3cccc4oc5c(-c6ccccc6-c6ccccc6)cc6ccccc6c5c34)cc2)cc1 ; c1ccc(-c2ccc(N(c3cccc4oc5ccccc5c34)c3cccc4oc5c(-c6ccccc6-c6ccccc6)cc6ccccc6c5c34)cc2)cc1 ; c1ccc(-c2ccccc2-c2cc3ccccc3c3c2oc2cccc(-c4ccc(N(c5ccccc5)c5ccccc5)cc4)c23)cc1]

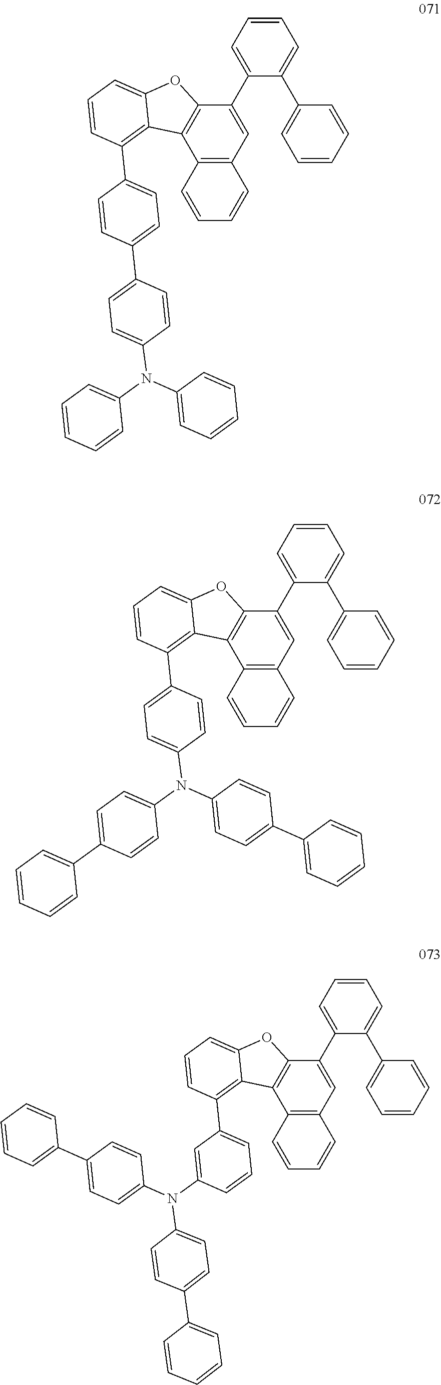 [SMILES: c1ccc(-c2ccccc2-c2cc3ccccc3c3c2oc2cccc(-c4ccc(-c5ccc(N(c6ccccc6)c6ccccc6)cc5)cc4)c23)cc1 ; c1ccc(-c2ccc(N(c3ccc(-c4ccccc4)cc3)c3ccc(-c4cccc5oc6c(-c7ccccc7-c7ccccc7)cc7ccccc7c6c45)cc3)cc2)cc1 ; c1ccc(-c2ccc(N(c3ccc(-c4ccccc4)cc3)c3cccc(-c4cccc5oc6c(-c7ccccc7-c7ccccc7)cc7ccccc7c6c45)c3)cc2)cc1]

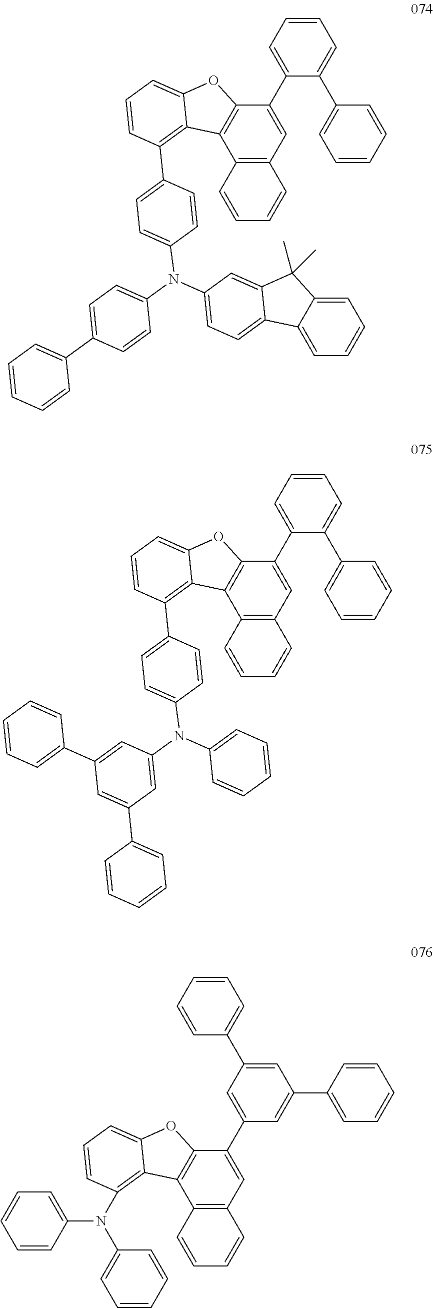 [SMILES: CC1(C)c2ccccc2-c2ccc(N(c3ccc(-c4ccccc4)cc3)c3ccc(-c4cccc5oc6c(-c7ccccc7-c7ccccc7)cc7ccccc7c6c45)cc3)cc21 ; c1ccc(-c2cc(-c3ccccc3)cc(N(c3ccccc3)c3ccc(-c4cccc5oc6c(-c7ccccc7-c7ccccc7)cc7ccccc7c6c45)cc3)c2)cc1 ; c1ccc(-c2cc(-c3ccccc3)cc(-c3cc4ccccc4c4c3oc3cccc(N(c5ccccc5)c5ccccc5)c34)c2)cc1]

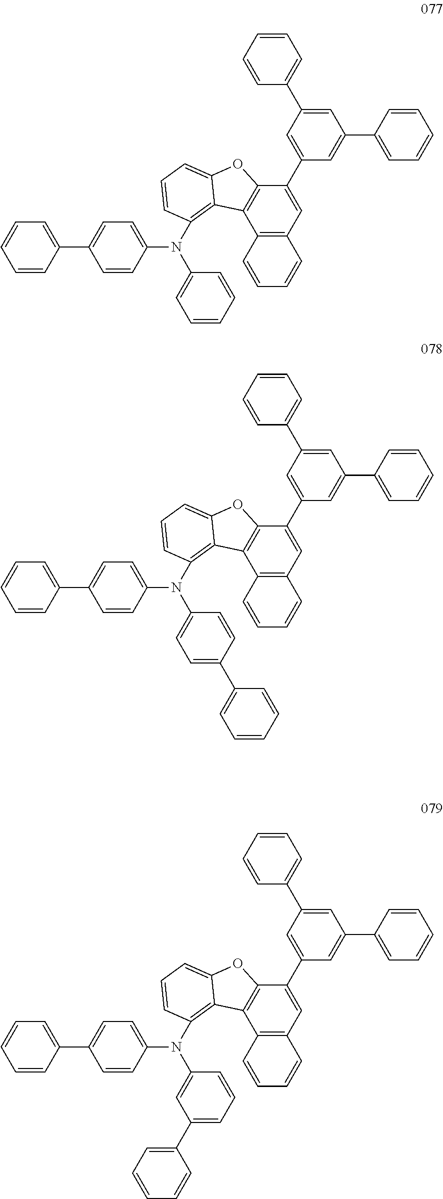 [SMILES: c1ccc(-c2ccc(N(c3ccccc3)c3cccc4oc5c(-c6cc(-c7ccccc7)cc(-c7ccccc7)c6)cc6ccccc6c5c34)cc2)cc1 ; c1ccc(-c2ccc(N(c3ccc(-c4ccccc4)cc3)c3cccc4oc5c(-c6cc(-c7ccccc7)cc(-c7ccccc7)c6)cc6ccccc6c5c34)cc2)cc1 ; c1ccc(-c2ccc(N(c3cccc(-c4ccccc4)c3)c3cccc4oc5c(-c6cc(-c7ccccc7)cc(-c7ccccc7)c6)cc6ccccc6c5c34)cc2)cc1]

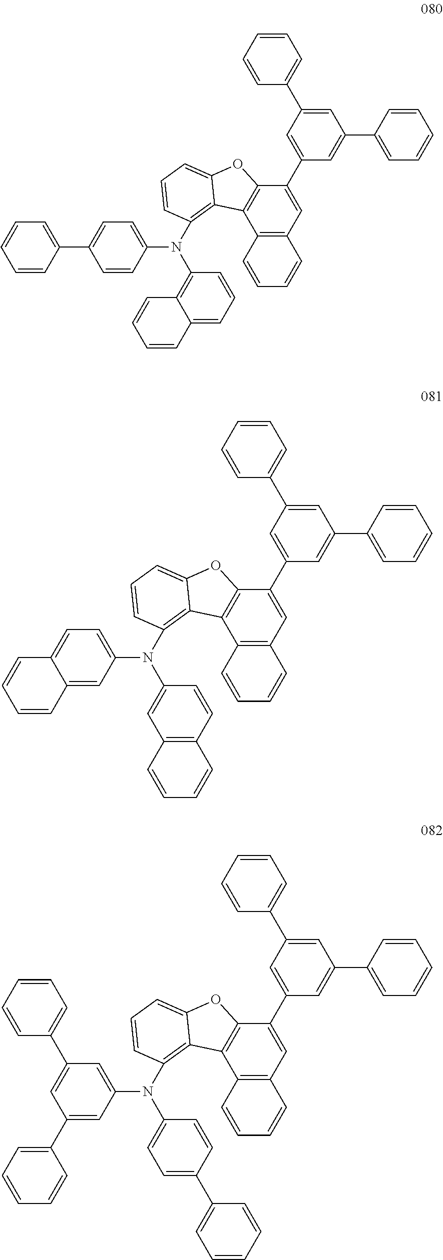 [SMILES: c1ccc(-c2ccc(N(c3cccc4ccccc34)c3cccc4oc5c(-c6cc(-c7ccccc7)cc(-c7ccccc7)c6)cc6ccccc6c5c34)cc2)cc1 ; c1ccc(-c2cc(-c3ccccc3)cc(-c3cc4ccccc4c4c3oc3cccc(N(c5ccc6ccccc6c5)c5ccc6ccccc6c5)c34)c2)cc1 ; c1ccc(-c2ccc(N(c3cc(-c4ccccc4)cc(-c4ccccc4)c3)c3cccc4oc5c(-c6cc(-c7ccccc7)cc(-c7ccccc7)c6)cc6ccccc6c5c34)cc2)cc1]

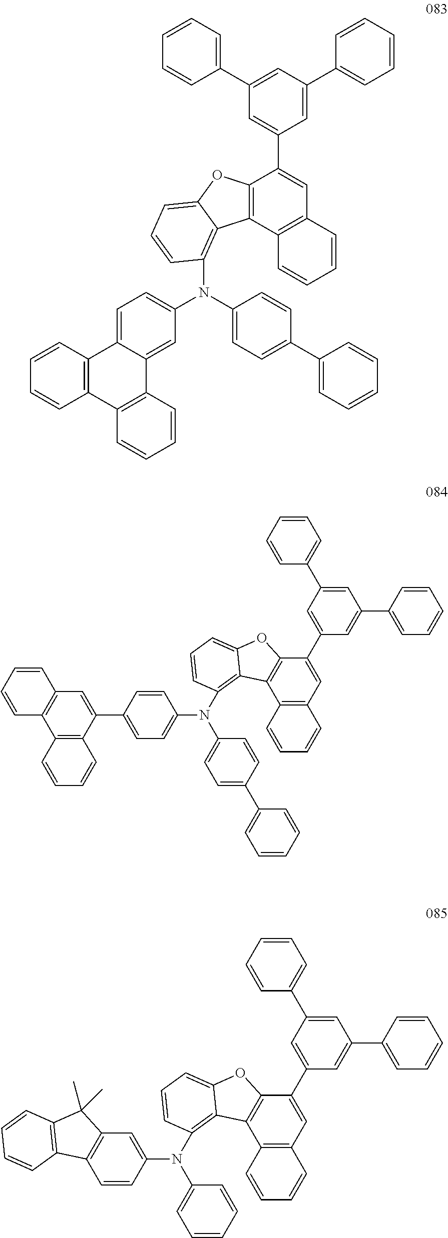 [SMILES: c1ccc(-c2ccc(N(c3ccc4c5ccccc5c5ccccc5c4c3)c3cccc4oc5c(-c6cc(-c7ccccc7)cc(-c7ccccc7)c6)cc6ccccc6c5c34)cc2)cc1 ; c1ccc(-c2ccc(N(c3ccc(-c4cc5ccccc5c5ccccc45)cc3)c3cccc4oc5c(-c6cc(-c7ccccc7)cc(-c7ccccc7)c6)cc6ccccc6c5c34)cc2)cc1 ; CC1(C)c2ccccc2-c2ccc(N(c3ccccc3)c3cccc4oc5c(-c6cc(-c7ccccc7)cc(-c7ccccc7)c6)cc6ccccc6c5c34)cc21]

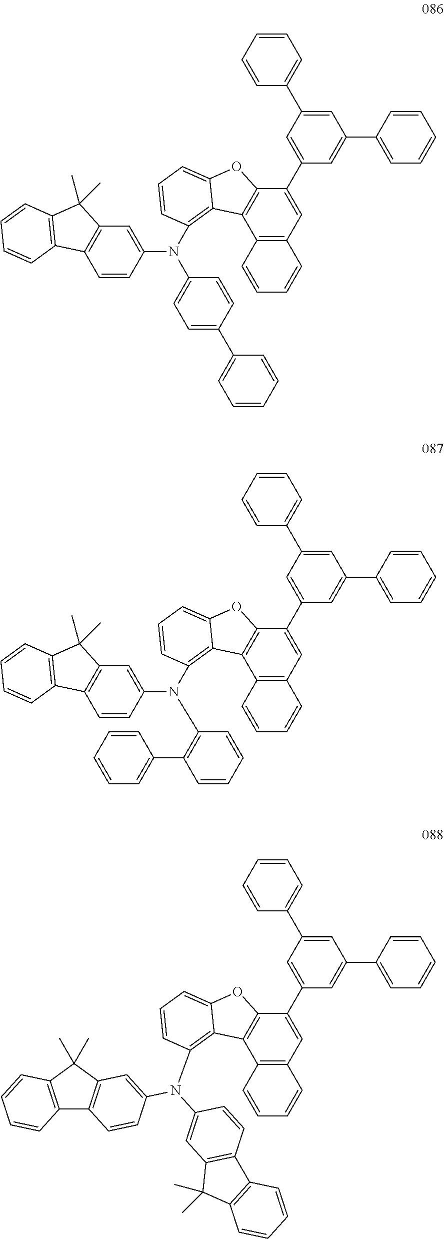 [SMILES: CC1(C)c2ccccc2-c2ccc(N(c3ccc(-c4ccccc4)cc3)c3cccc4oc5c(-c6cc(-c7ccccc7)cc(-c7ccccc7)c6)cc6ccccc6c5c34)cc21 ; CC1(C)c2ccccc2-c2ccc(N(c3ccccc3-c3ccccc3)c3cccc4oc5c(-c6cc(-c7ccccc7)cc(-c7ccccc7)c6)cc6ccccc6c5c34)cc21 ; CC1(C)c2ccccc2-c2ccc(N(c3ccc4c(c3)C(C)(C)c3ccccc3-4)c3cccc4oc5c(-c6cc(-c7ccccc7)cc(-c7ccccc7)c6)cc6ccccc6c5c34)cc21]

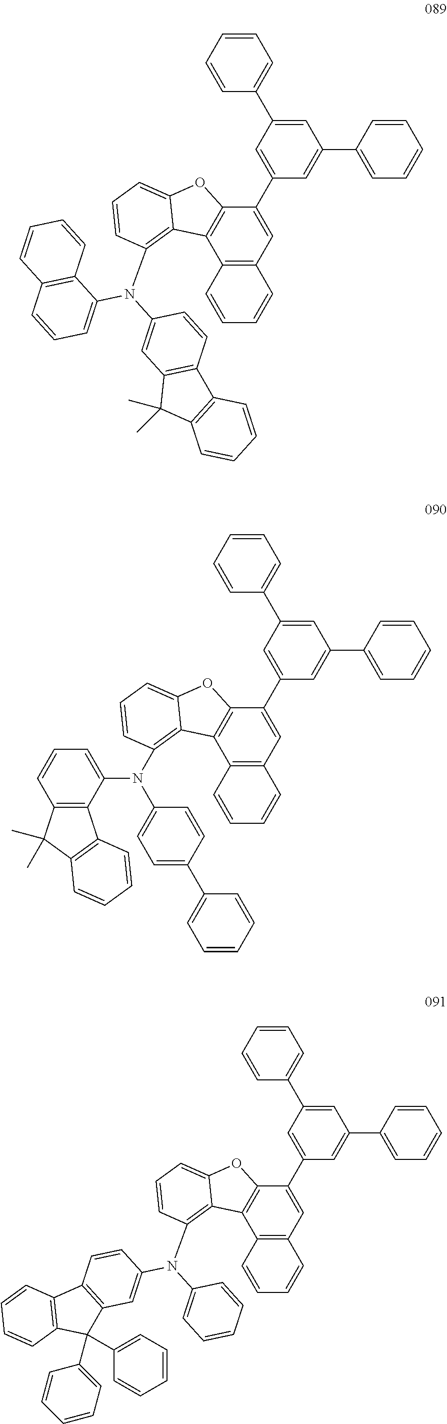 [SMILES: CC1(C)c2ccccc2-c2ccc(N(c3cccc4ccccc34)c3cccc4oc5c(-c6cc(-c7ccccc7)cc(-c7ccccc7)c6)cc6ccccc6c5c34)cc21 ; CC1(C)c2ccccc2-c2c(N(c3ccc(-c4ccccc4)cc3)c3cccc4oc5c(-c6cc(-c7ccccc7)cc(-c7ccccc7)c6)cc6ccccc6c5c34)cccc21 ; c1ccc(-c2cc(-c3ccccc3)cc(-c3cc4ccccc4c4c3oc3cccc(N(c5ccccc5)c5ccc6c(c5)C(c5ccccc5)(c5ccccc5)c5ccccc5-6)c34)c2)cc1]

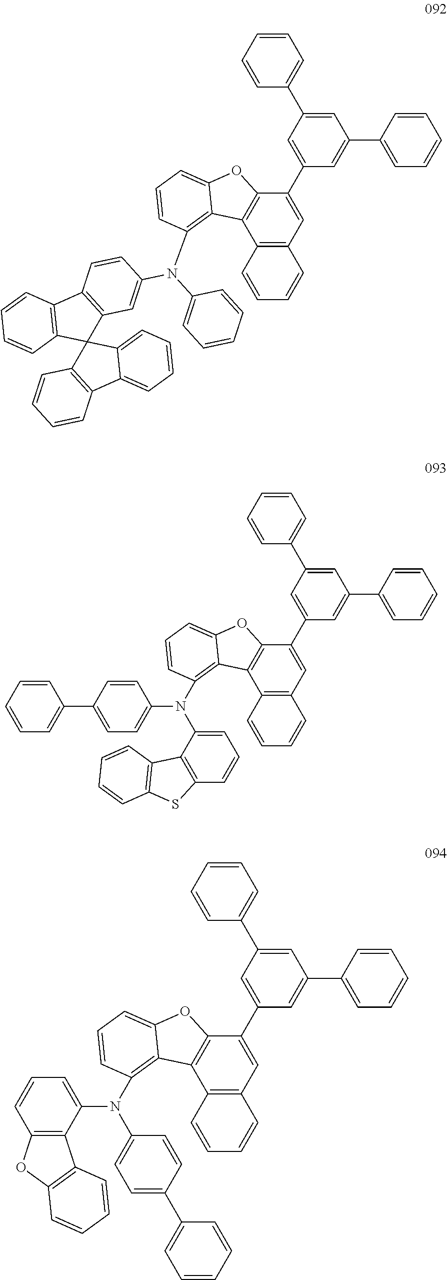 [SMILES: c1ccc(-c2cc(-c3ccccc3)cc(-c3cc4ccccc4c4c3oc3cccc(N(c5ccccc5)c5ccc6c(c5)C5(c7ccccc7-c7ccccc75)c5ccccc5-6)c34)c2)cc1 ; c1ccc(-c2ccc(N(c3cccc4sc5ccccc5c34)c3cccc4oc5c(-c6cc(-c7ccccc7)cc(-c7ccccc7)c6)cc6ccccc6c5c34)cc2)cc1 ; c1ccc(-c2ccc(N(c3cccc4oc5ccccc5c34)c3cccc4oc5c(-c6cc(-c7ccccc7)cc(-c7ccccc7)c6)cc6ccccc6c5c34)cc2)cc1]

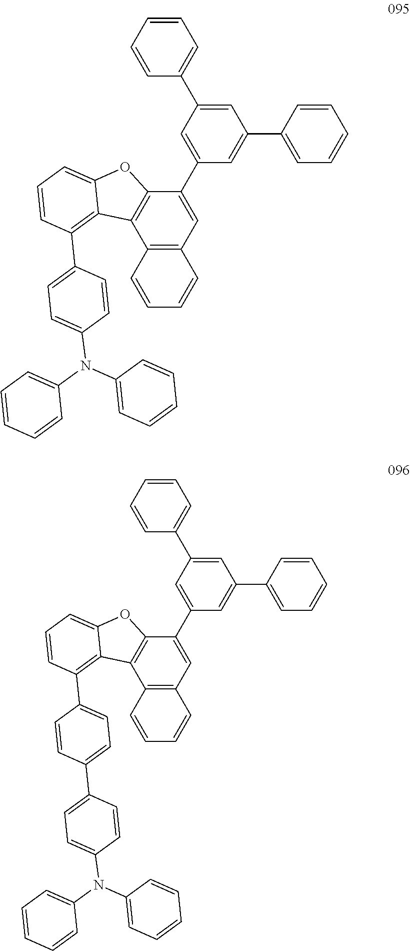 [SMILES: c1ccc(-c2cc(-c3ccccc3)cc(-c3cc4ccccc4c4c3oc3cccc(-c5ccc(N(c6ccccc6)c6ccccc6)cc5)c34)c2)cc1 ; c1ccc(-c2cc(-c3ccccc3)cc(-c3cc4ccccc4c4c3oc3cccc(-c5ccc(-c6ccc(N(c7ccccc7)c7ccccc7)cc6)cc5)c34)c2)cc1]

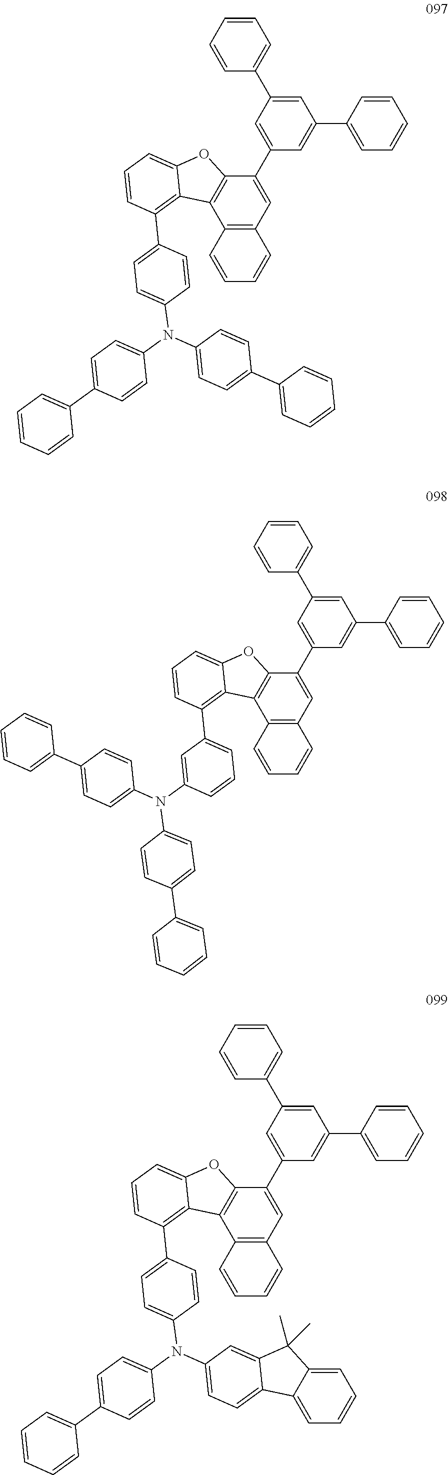 [SMILES: c1ccc(-c2ccc(N(c3ccc(-c4ccccc4)cc3)c3ccc(-c4cccc5oc6c(-c7cc(-c8ccccc8)cc(-c8ccccc8)c7)cc7ccccc7c6c45)cc3)cc2)cc1 ; c1ccc(-c2ccc(N(c3ccc(-c4ccccc4)cc3)c3cccc(-c4cccc5oc6c(-c7cc(-c8ccccc8)cc(-c8ccccc8)c7)cc7ccccc7c6c45)c3)cc2)cc1 ; CC1(C)c2ccccc2-c2ccc(N(c3ccc(-c4ccccc4)cc3)c3ccc(-c4cccc5oc6c(-c7cc(-c8ccccc8)cc(-c8ccccc8)c7)cc7ccccc7c6c45)cc3)cc21]

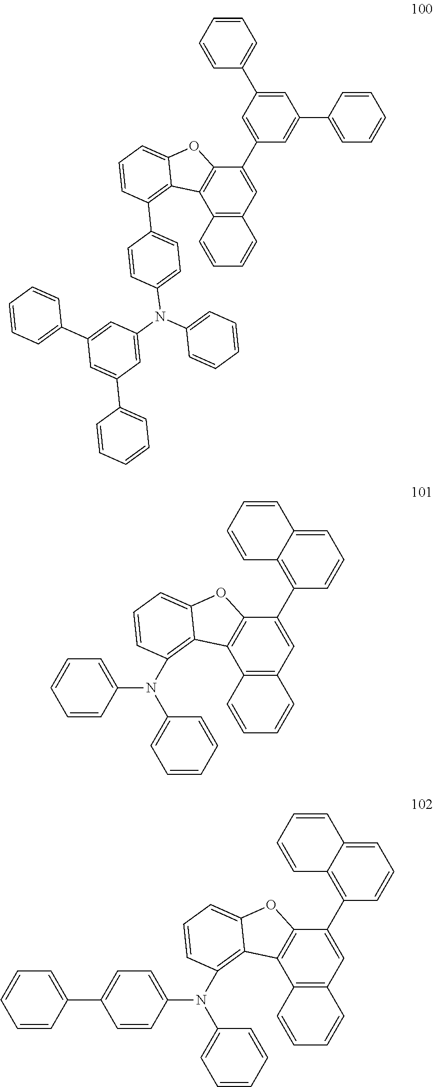 [SMILES: c1ccc(-c2cc(-c3ccccc3)cc(-c3cc4ccccc4c4c3oc3cccc(-c5ccc(N(c6ccccc6)c6cc(-c7ccccc7)cc(-c7ccccc7)c6)cc5)c34)c2)cc1 ; c1ccc(N(c2ccccc2)c2cccc3oc4c(-c5cccc6ccccc56)cc5ccccc5c4c23)cc1 ; c1ccc(-c2ccc(N(c3ccccc3)c3cccc4oc5c(-c6cccc7ccccc67)cc6ccccc6c5c34)cc2)cc1]

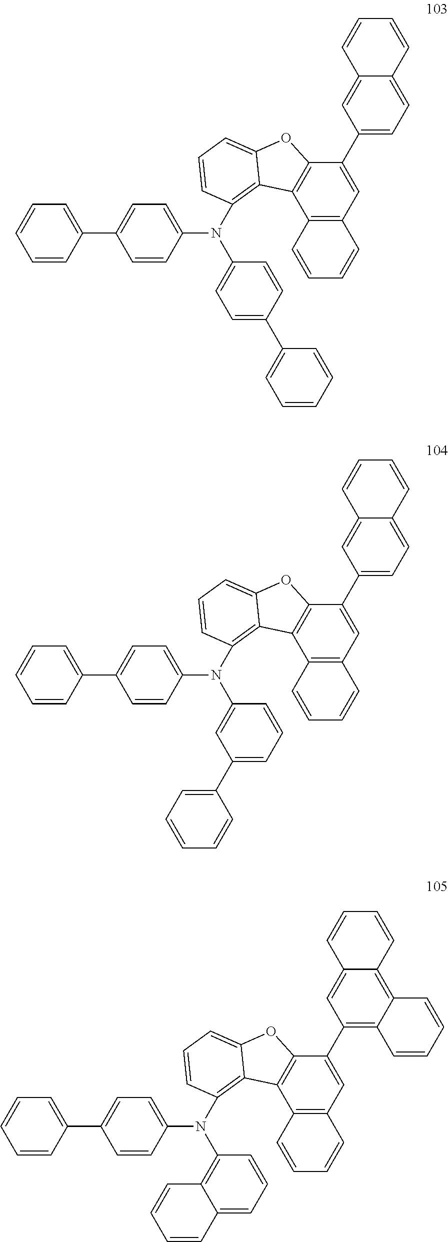 [SMILES: c1ccc(-c2ccc(N(c3ccc(-c4ccccc4)cc3)c3cccc4oc5c(-c6ccc7ccccc7c6)cc6ccccc6c5c34)cc2)cc1 ; c1ccc(-c2ccc(N(c3cccc(-c4ccccc4)c3)c3cccc4oc5c(-c6ccc7ccccc7c6)cc6ccccc6c5c34)cc2)cc1 ; c1ccc(-c2ccc(N(c3cccc4ccccc34)c3cccc4oc5c(-c6cc7ccccc7c7ccccc67)cc6ccccc6c5c34)cc2)cc1]

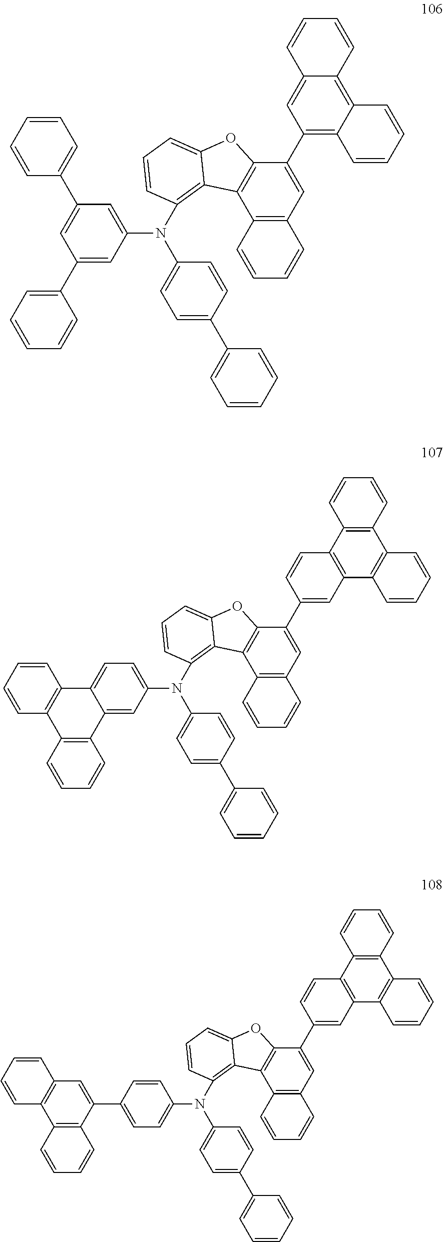 [SMILES: c1ccc(-c2ccc(N(c3cc(-c4ccccc4)cc(-c4ccccc4)c3)c3cccc4oc5c(-c6cc7ccccc7c7ccccc67)cc6ccccc6c5c34)cc2)cc1 ; c1ccc(-c2ccc(N(c3ccc4c5ccccc5c5ccccc5c4c3)c3cccc4oc5c(-c6ccc7c8ccccc8c8ccccc8c7c6)cc6ccccc6c5c34)cc2)cc1 ; c1ccc(-c2ccc(N(c3ccc(-c4cc5ccccc5c5ccccc45)cc3)c3cccc4oc5c(-c6ccc7c8ccccc8c8ccccc8c7c6)cc6ccccc6c5c34)cc2)cc1]

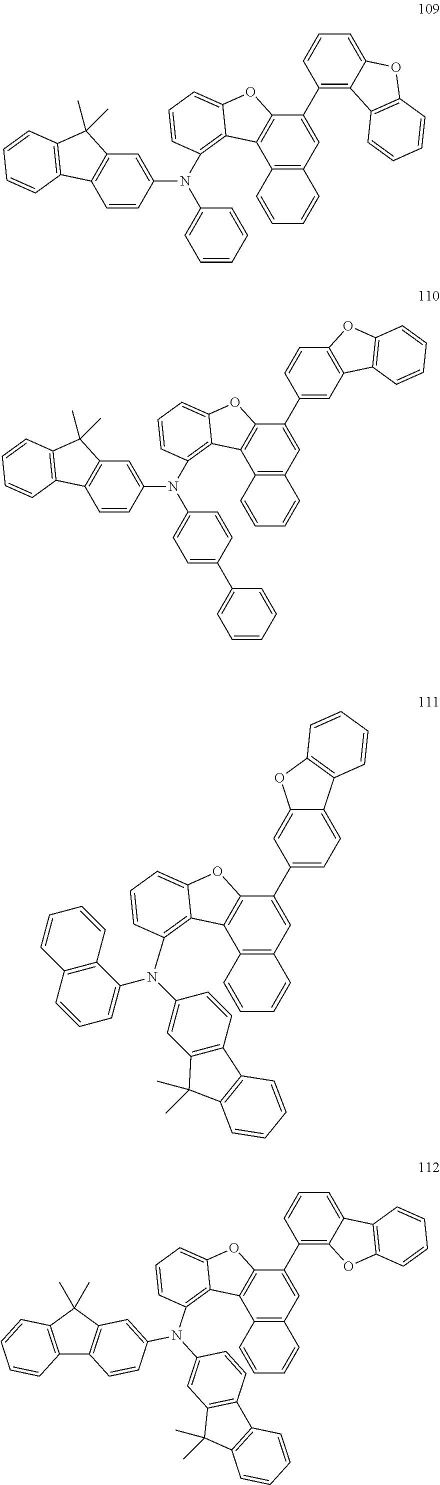 [SMILES: CC1(C)c2ccccc2-c2ccc(N(c3ccccc3)c3cccc4oc5c(-c6cccc7oc8ccccc8c67)cc6ccccc6c5c34)cc21 ; CC1(C)c2ccccc2-c2ccc(N(c3ccc(-c4ccccc4)cc3)c3cccc4oc5c(-c6ccc7oc8ccccc8c7c6)cc6ccccc6c5c34)cc21 ; CC1(C)c2ccccc2-c2ccc(N(c3cccc4ccccc34)c3cccc4oc5c(-c6ccc7c(c6)oc6ccccc67)cc6ccccc6c5c34)cc21 ; CC1(C)c2ccccc2-c2ccc(N(c3ccc4c(c3)C(C)(C)c3ccccc3-4)c3cccc4oc5c(-c6cccc7c6oc6ccccc67)cc6ccccc6c5c34)cc21]

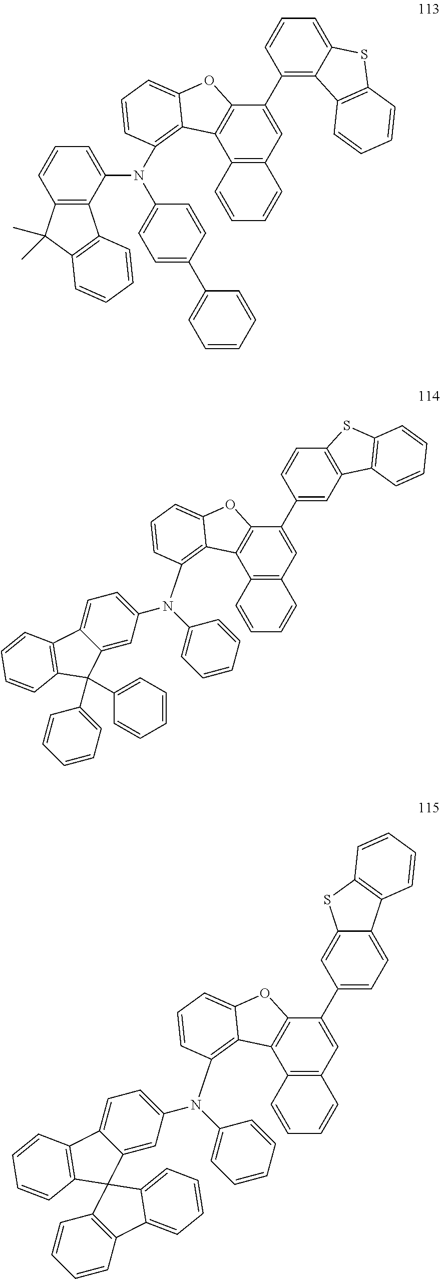 [SMILES: CC1(C)c2ccccc2-c2c(N(c3ccc(-c4ccccc4)cc3)c3cccc4oc5c(-c6cccc7sc8ccccc8c67)cc6ccccc6c5c34)cccc21 ; c1ccc(N(c2ccc3c(c2)C(c2ccccc2)(c2ccccc2)c2ccccc2-3)c2cccc3oc4c(-c5ccc6sc7ccccc7c6c5)cc5ccccc5c4c23)cc1 ; c1ccc(N(c2ccc3c(c2)C2(c4ccccc4-c4ccccc42)c2ccccc2-3)c2cccc3oc4c(-c5ccc6c(c5)sc5ccccc56)cc5ccccc5c4c23)cc1]

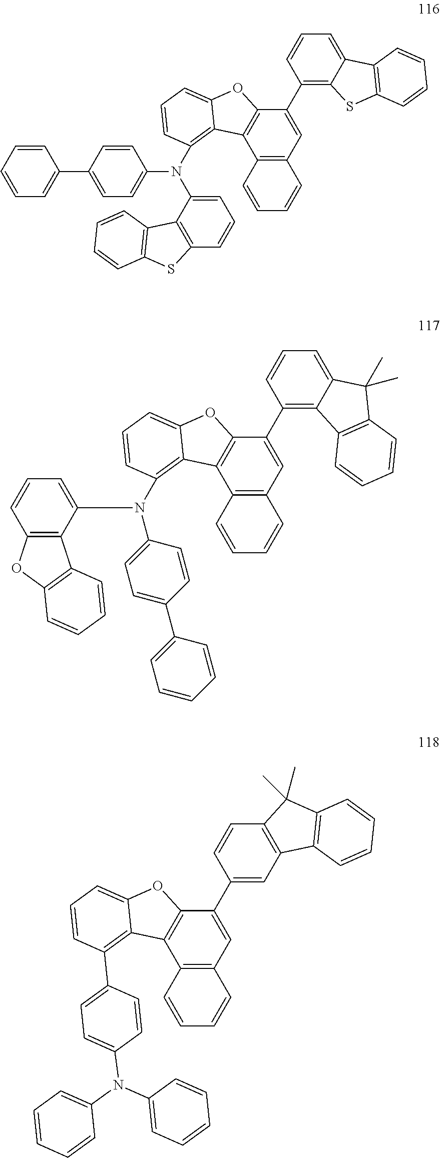 [SMILES: c1ccc(-c2ccc(N(c3cccc4sc5ccccc5c34)c3cccc4oc5c(-c6cccc7c6sc6ccccc67)cc6ccccc6c5c34)cc2)cc1 ; CC1(C)c2ccccc2-c2c(-c3cc4ccccc4c4c3oc3cccc(N(c5ccc(-c6ccccc6)cc5)c5cccc6oc7ccccc7c56)c34)cccc21 ; CC1(C)c2ccccc2-c2cc(-c3cc4ccccc4c4c3oc3cccc(-c5ccc(N(c6ccccc6)c6ccccc6)cc5)c34)ccc21]

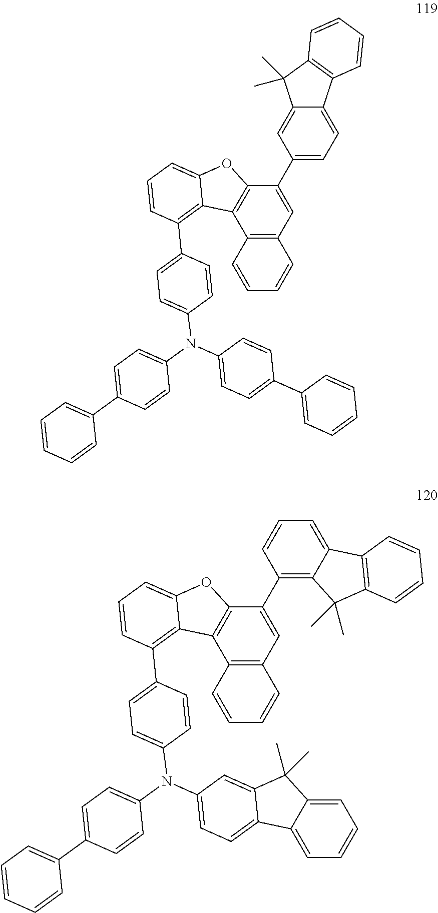 [SMILES: CC1(C)c2ccccc2-c2ccc(-c3cc4ccccc4c4c3oc3cccc(-c5ccc(N(c6ccc(-c7ccccc7)cc6)c6ccc(-c7ccccc7)cc6)cc5)c34)cc21 ; CC1(C)c2ccccc2-c2ccc(N(c3ccc(-c4ccccc4)cc3)c3ccc(-c4cccc5oc6c(-c7cccc8c7C(C)(C)c7ccccc7-8)cc7ccccc7c6c45)cc3)cc21]

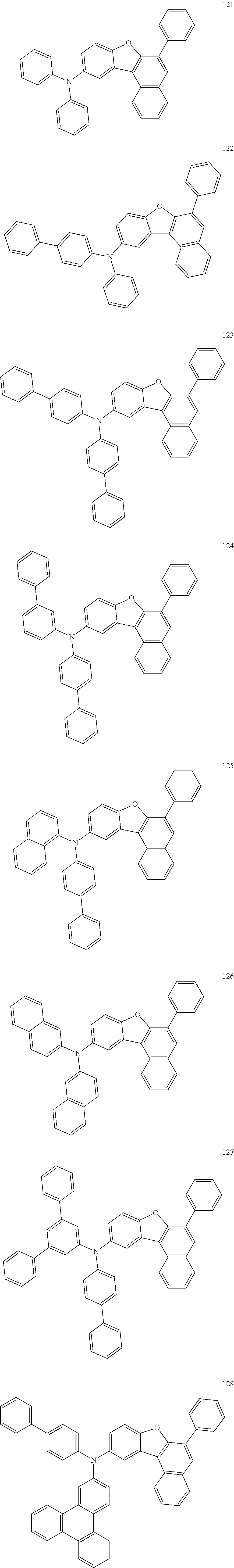

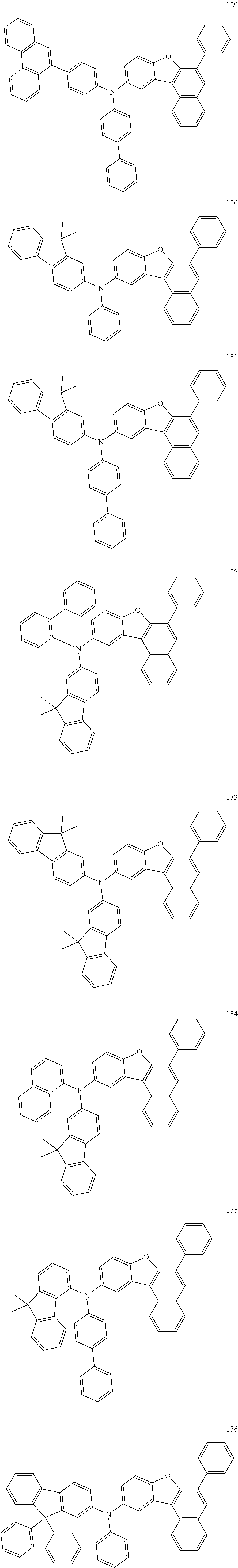

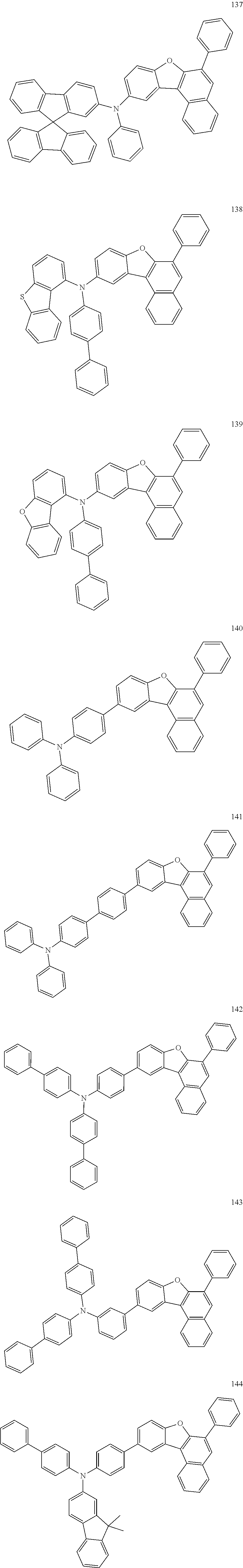

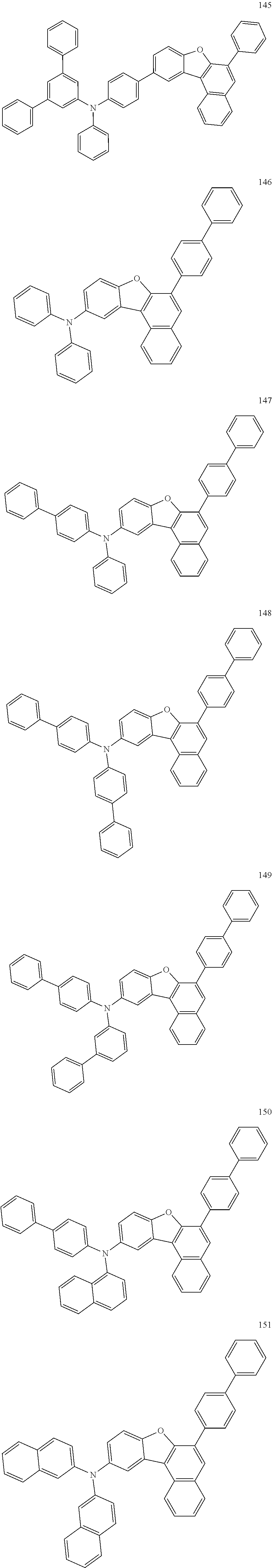

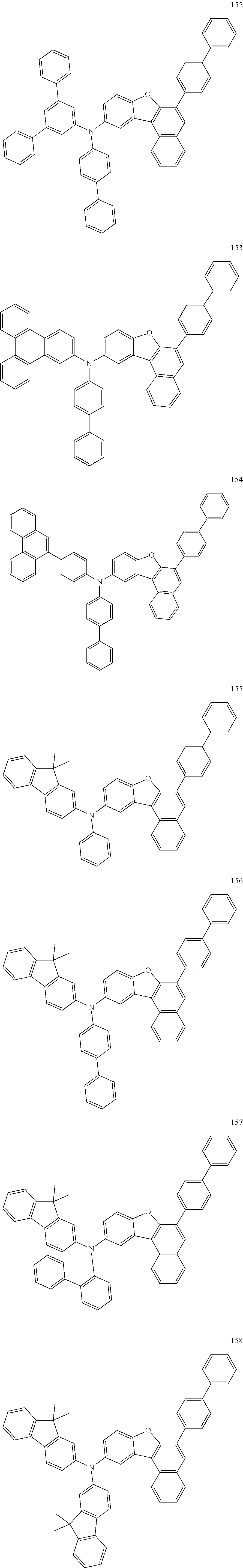

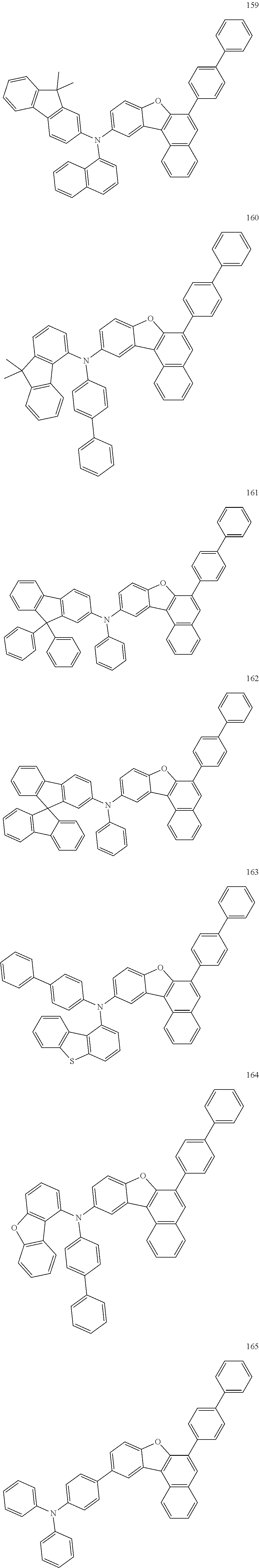

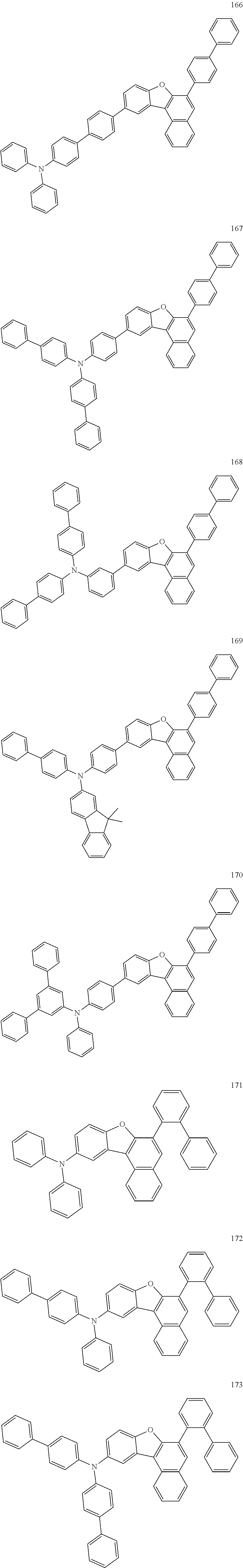

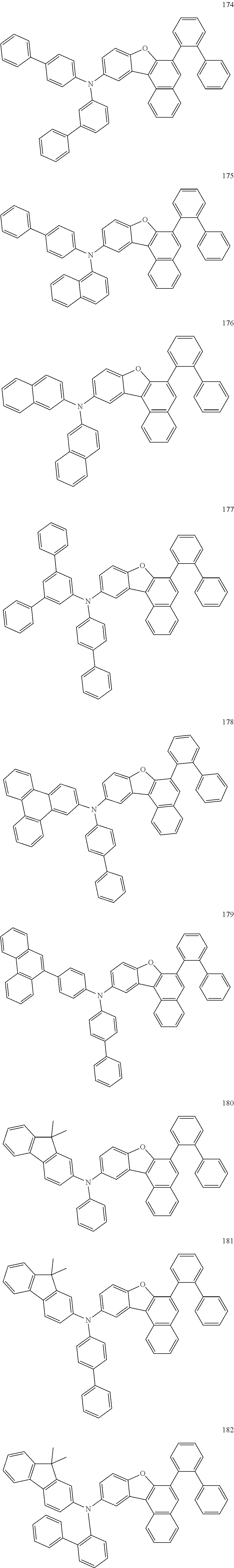

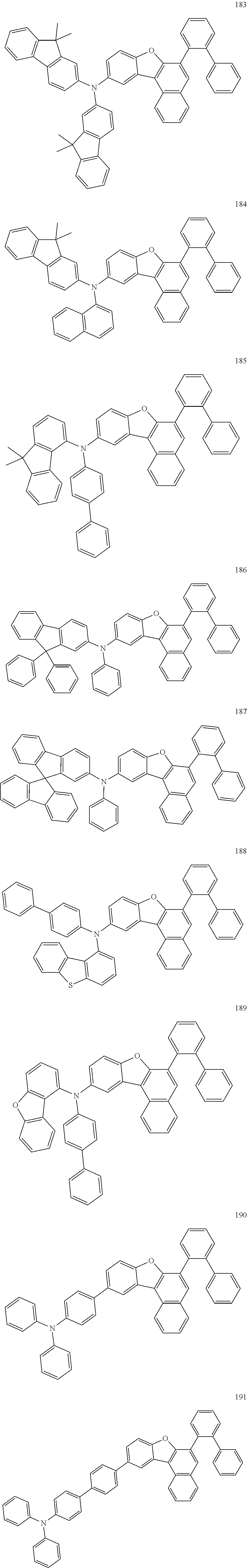

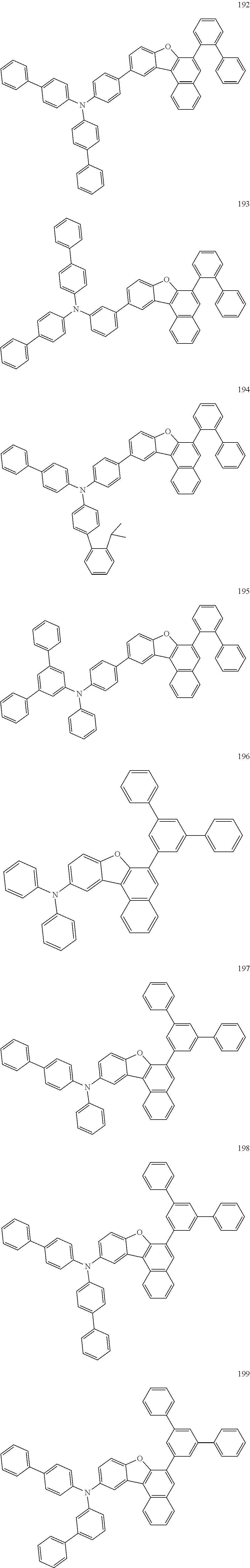

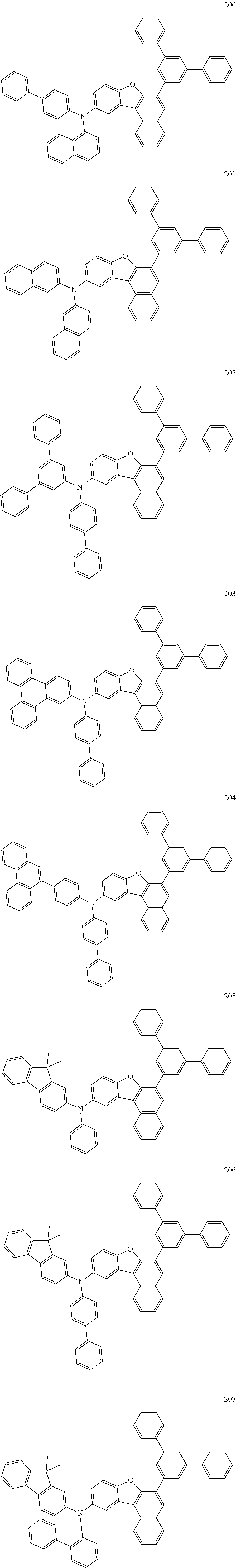

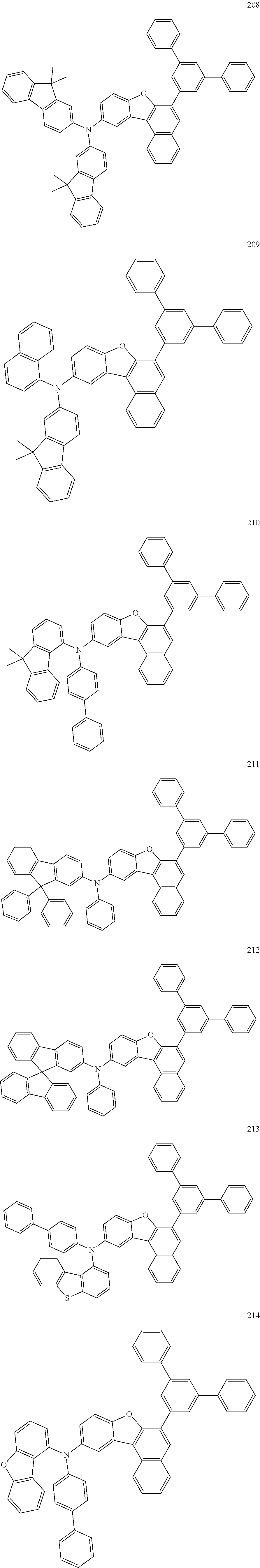

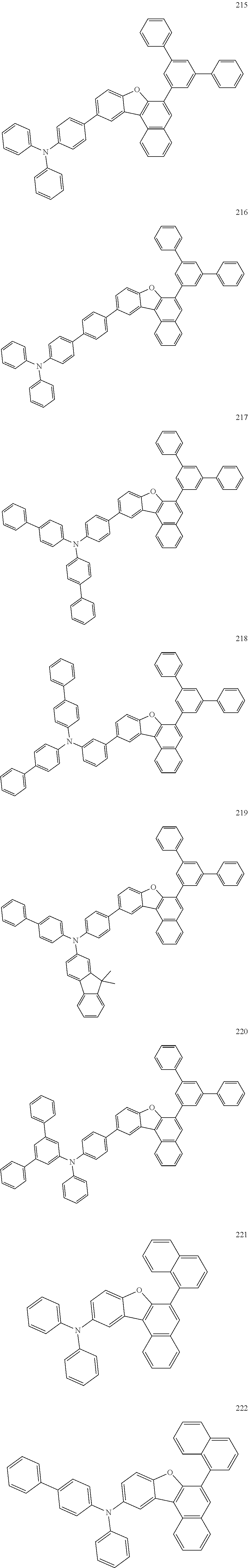

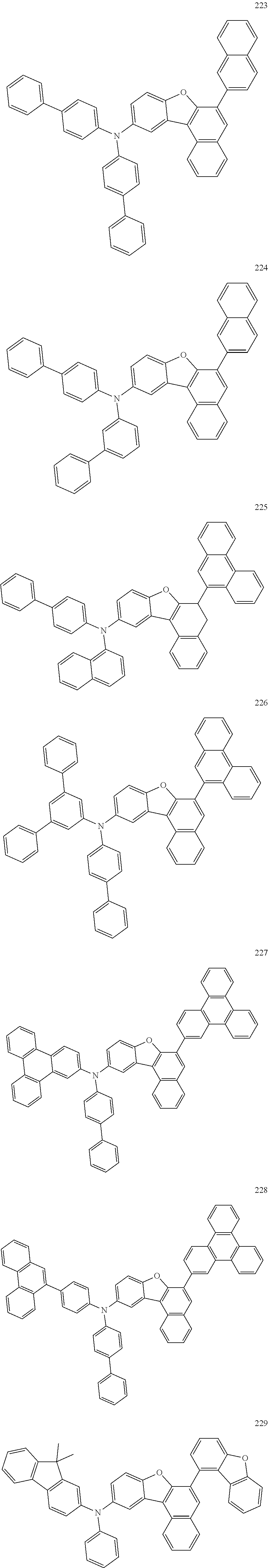

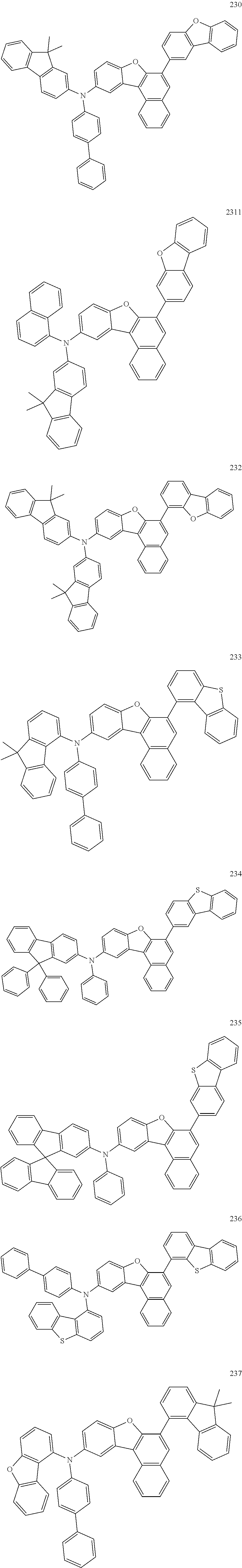

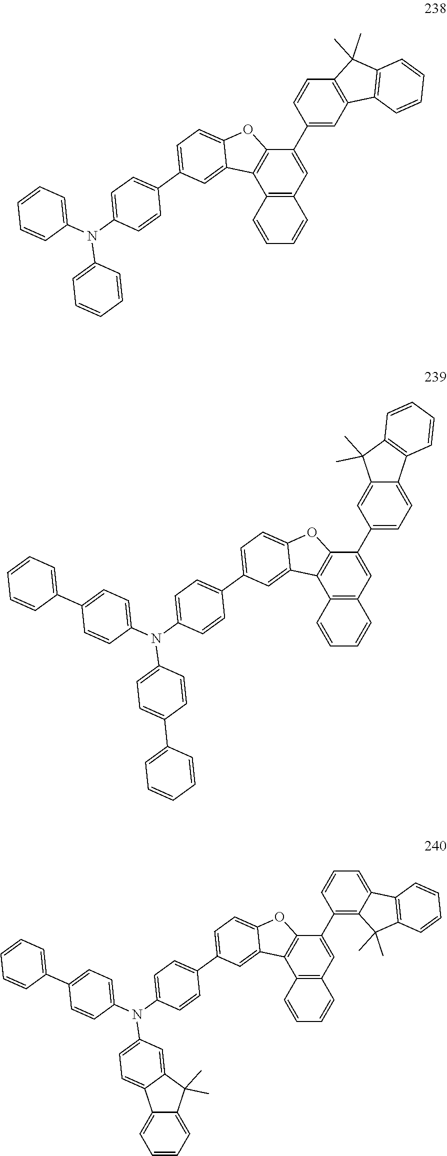 [SMILES: CC1(C)c2ccccc2-c2cc(-c3cc4ccccc4c4c3oc3ccc(-c5ccc(N(c6ccccc6)c6ccccc6)cc5)cc34)ccc21 ; CC1(C)c2ccccc2-c2ccc(-c3cc4ccccc4c4c3oc3ccc(-c5ccc(N(c6ccc(-c7ccccc7)cc6)c6ccc(-c7ccccc7)cc6)cc5)cc34)cc21 ; CC1(C)c2ccccc2-c2ccc(N(c3ccc(-c4ccccc4)cc3)c3ccc(-c4ccc5oc6c(-c7cccc8c7C(C)(C)c7ccccc7-8)cc7ccccc7c6c5c4)cc3)cc21]

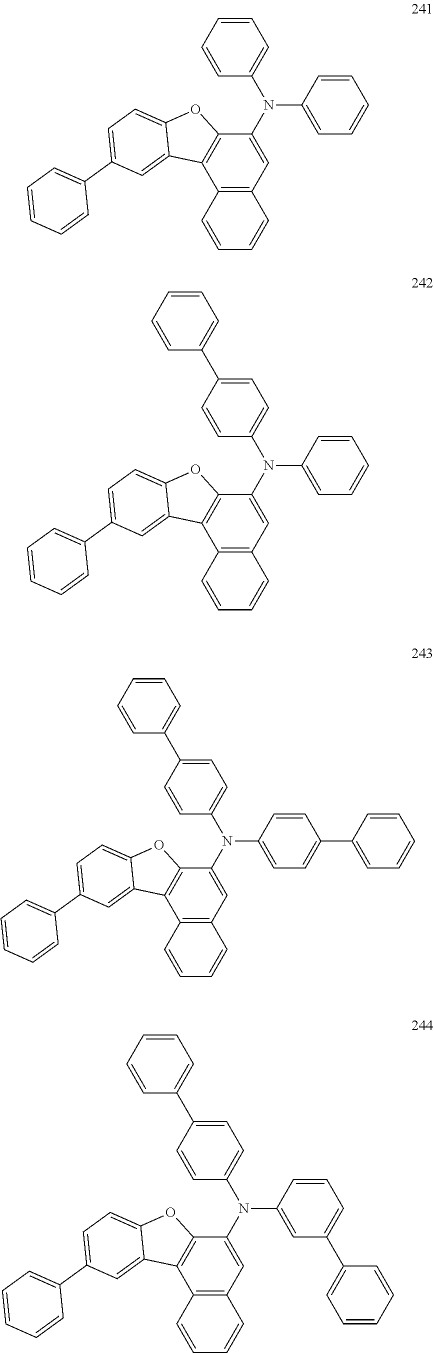

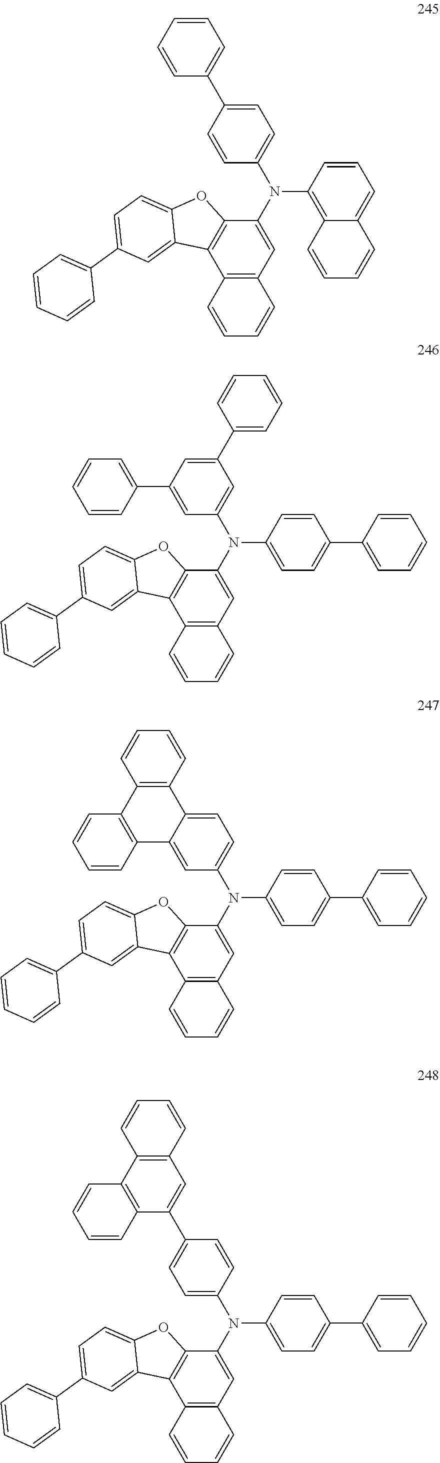

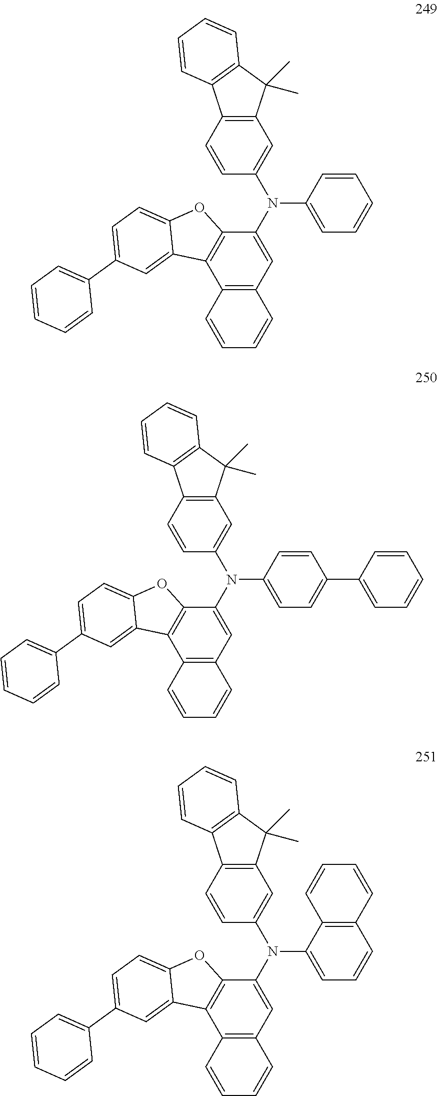 [SMILES: CC1(C)c2ccccc2-c2ccc(N(c3ccccc3)c3cc4ccccc4c4c3oc3ccc(-c5ccccc5)cc34)cc21 ; CC1(C)c2ccccc2-c2ccc(N(c3ccc(-c4ccccc4)cc3)c3cc4ccccc4c4c3oc3ccc(-c5ccccc5)cc34)cc21 ; CC1(C)c2ccccc2-c2ccc(N(c3cccc4ccccc34)c3cc4ccccc4c4c3oc3ccc(-c5ccccc5)cc34)cc21]

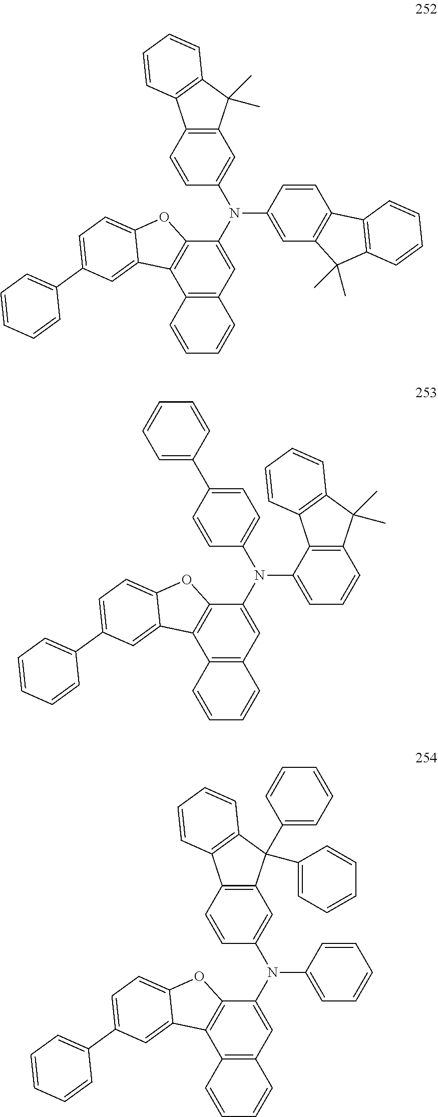 [SMILES: CC1(C)c2ccccc2-c2ccc(N(c3ccc4c(c3)C(C)(C)c3ccccc3-4)c3cc4ccccc4c4c3oc3ccc(-c5ccccc5)cc34)cc21 ; CC1(C)c2ccccc2-c2c(N(c3ccc(-c4ccccc4)cc3)c3cc4ccccc4c4c3oc3ccc(-c5ccccc5)cc34)cccc21 ; c1ccc(-c2ccc3oc4c(N(c5ccccc5)c5ccc6c(c5)C(c5ccccc5)(c5ccccc5)c5ccccc5-6)cc5ccccc5c4c3c2)cc1]

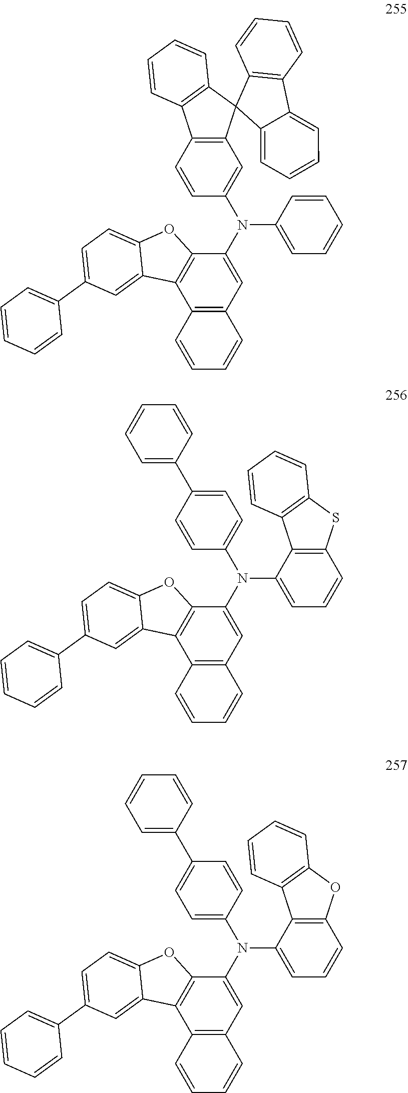

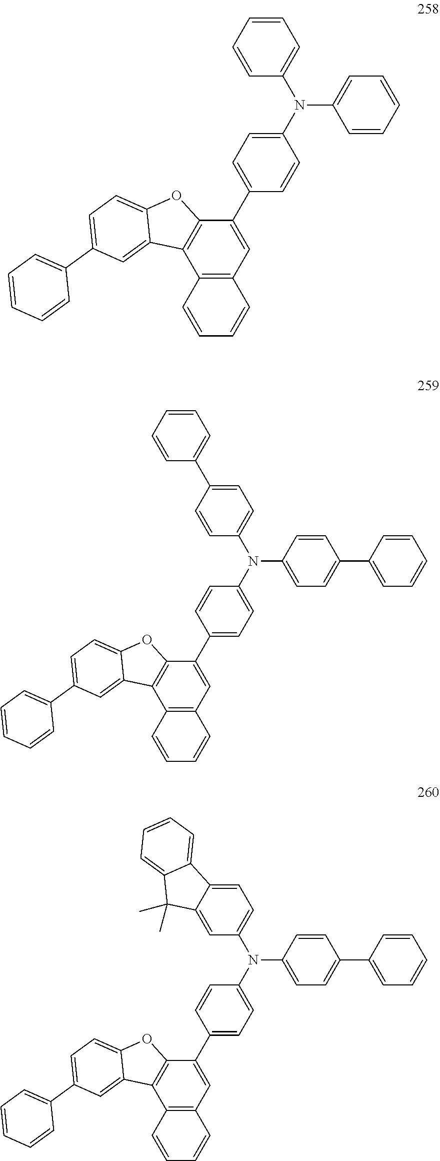 [SMILES: c1ccc(-c2ccc3oc4c(-c5ccc(N(c6ccccc6)c6ccccc6)cc5)cc5ccccc5c4c3c2)cc1 ; c1ccc(-c2ccc(N(c3ccc(-c4ccccc4)cc3)c3ccc(-c4cc5ccccc5c5c4oc4ccc(-c6ccccc6)cc45)cc3)cc2)cc1 ; CC1(C)c2ccccc2-c2ccc(N(c3ccc(-c4ccccc4)cc3)c3ccc(-c4cc5ccccc5c5c4oc4ccc(-c6ccccc6)cc45)cc3)cc21]

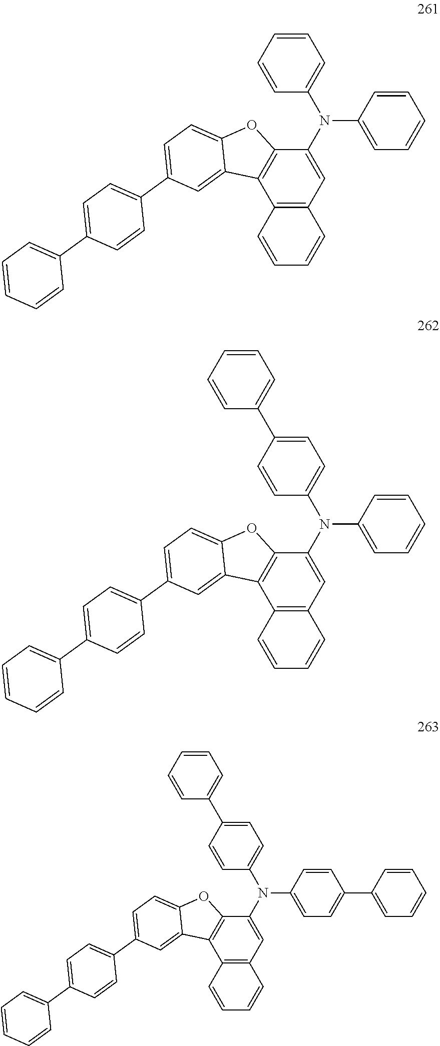 [SMILES: c1ccc(-c2ccc(-c3ccc4oc5c(N(c6ccccc6)c6ccccc6)cc6ccccc6c5c4c3)cc2)cc1 ; c1ccc(-c2ccc(-c3ccc4oc5c(N(c6ccccc6)c6ccc(-c7ccccc7)cc6)cc6ccccc6c5c4c3)cc2)cc1 ; c1ccc(-c2ccc(-c3ccc4oc5c(N(c6ccc(-c7ccccc7)cc6)c6ccc(-c7ccccc7)cc6)cc6ccccc6c5c4c3)cc2)cc1]

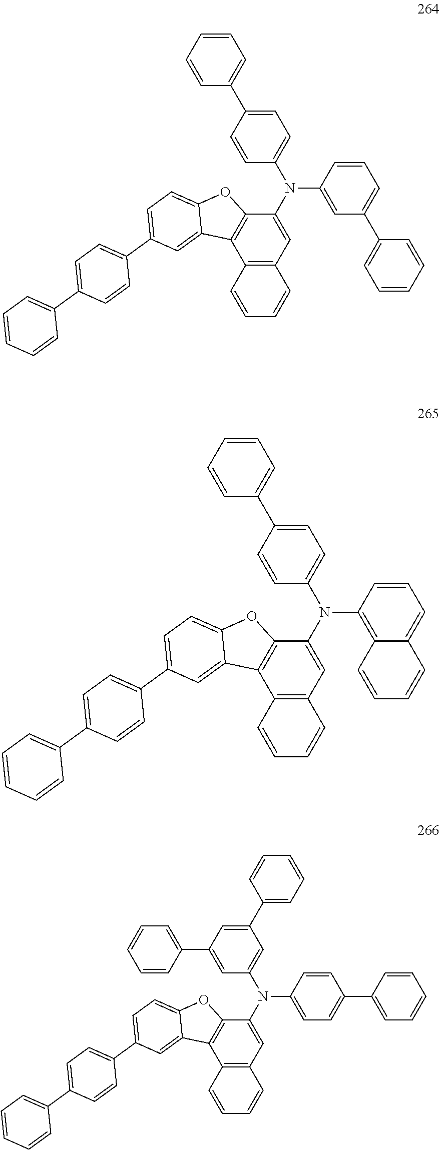 [SMILES: c1ccc(-c2ccc(-c3ccc4oc5c(N(c6ccc(-c7ccccc7)cc6)c6cccc(-c7ccccc7)c6)cc6ccccc6c5c4c3)cc2)cc1 ; c1ccc(-c2ccc(-c3ccc4oc5c(N(c6ccc(-c7ccccc7)cc6)c6cccc7ccccc67)cc6ccccc6c5c4c3)cc2)cc1 ; c1ccc(-c2ccc(-c3ccc4oc5c(N(c6ccc(-c7ccccc7)cc6)c6cc(-c7ccccc7)cc(-c7ccccc7)c6)cc6ccccc6c5c4c3)cc2)cc1]

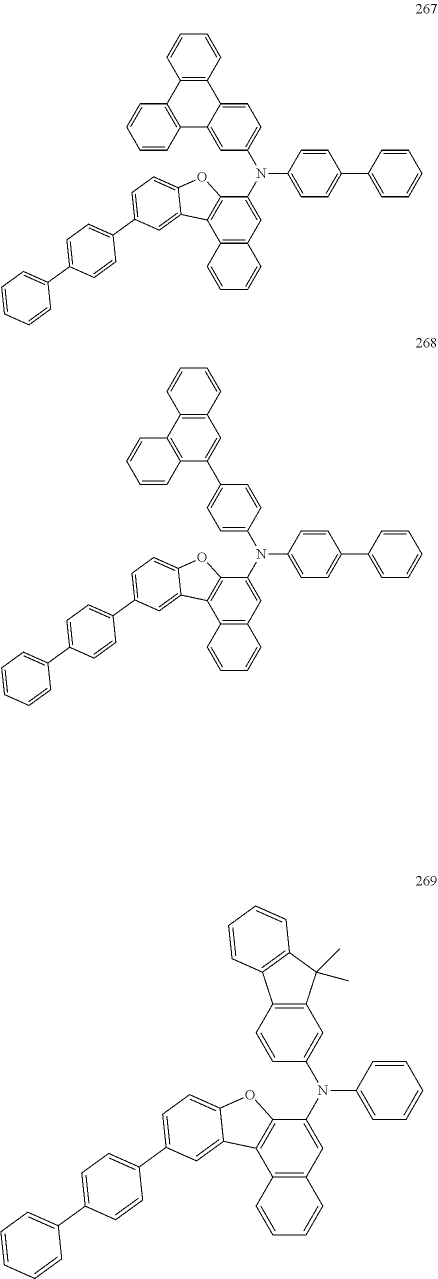 [SMILES: c1ccc(-c2ccc(-c3ccc4oc5c(N(c6ccc(-c7ccccc7)cc6)c6ccc7c8ccccc8c8ccccc8c7c6)cc6ccccc6c5c4c3)cc2)cc1 ; c1ccc(-c2ccc(-c3ccc4oc5c(N(c6ccc(-c7ccccc7)cc6)c6ccc(-c7cc8ccccc8c8ccccc78)cc6)cc6ccccc6c5c4c3)cc2)cc1 ; CC1(C)c2ccccc2-c2ccc(N(c3ccccc3)c3cc4ccccc4c4c3oc3ccc(-c5ccc(-c6ccccc6)cc5)cc34)cc21]

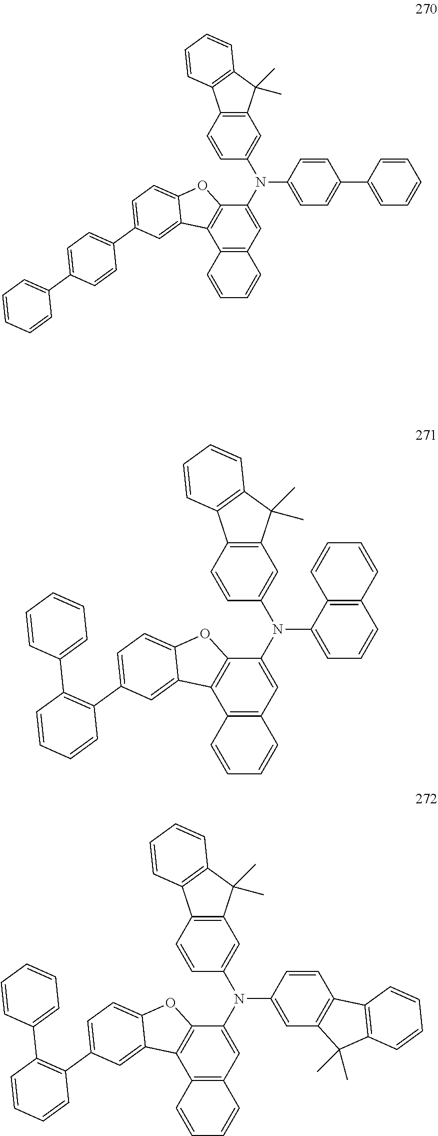 [SMILES: CC1(C)c2ccccc2-c2ccc(N(c3ccc(-c4ccccc4)cc3)c3cc4ccccc4c4c3oc3ccc(-c5ccc(-c6ccccc6)cc5)cc34)cc21 ; CC1(C)c2ccccc2-c2ccc(N(c3cccc4ccccc34)c3cc4ccccc4c4c3oc3ccc(-c5ccccc5-c5ccccc5)cc34)cc21 ; CC1(C)c2ccccc2-c2ccc(N(c3ccc4c(c3)C(C)(C)c3ccccc3-4)c3cc4ccccc4c4c3oc3ccc(-c5ccccc5-c5ccccc5)cc34)cc21]

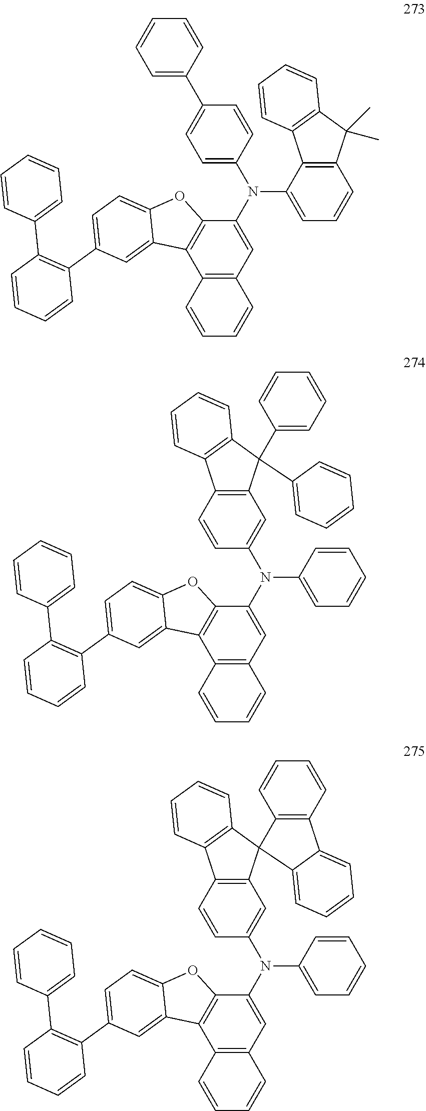 [SMILES: CC1(C)c2ccccc2-c2c(N(c3ccc(-c4ccccc4)cc3)c3cc4ccccc4c4c3oc3ccc(-c5ccccc5-c5ccccc5)cc34)cccc21 ; c1ccc(-c2ccccc2-c2ccc3oc4c(N(c5ccccc5)c5ccc6c(c5)C(c5ccccc5)(c5ccccc5)c5ccccc5-6)cc5ccccc5c4c3c2)cc1 ; c1ccc(-c2ccccc2-c2ccc3oc4c(N(c5ccccc5)c5ccc6c(c5)C5(c7ccccc7-c7ccccc75)c5ccccc5-6)cc5ccccc5c4c3c2)cc1]

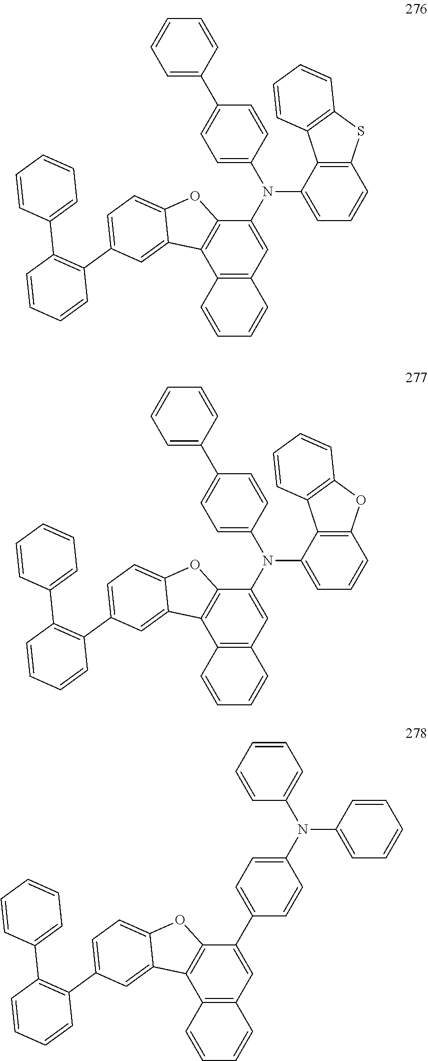

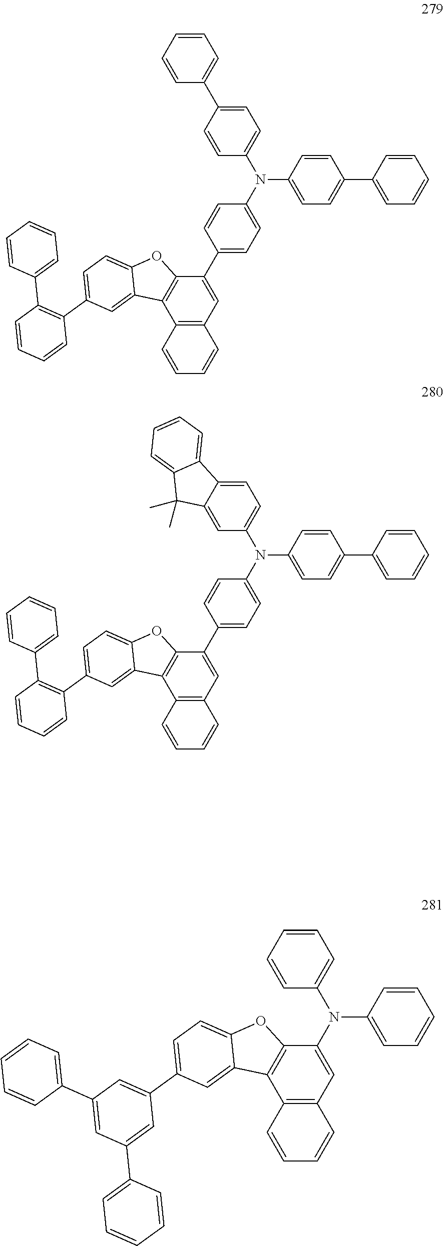 [SMILES: c1ccc(-c2ccc(N(c3ccc(-c4ccccc4)cc3)c3ccc(-c4cc5ccccc5c5c4oc4ccc(-c6ccccc6-c6ccccc6)cc45)cc3)cc2)cc1 ; CC1(C)c2ccccc2-c2ccc(N(c3ccc(-c4ccccc4)cc3)c3ccc(-c4cc5ccccc5c5c4oc4ccc(-c6ccccc6-c6ccccc6)cc45)cc3)cc21 ; c1ccc(-c2cc(-c3ccccc3)cc(-c3ccc4oc5c(N(c6ccccc6)c6ccccc6)cc6ccccc6c5c4c3)c2)cc1]

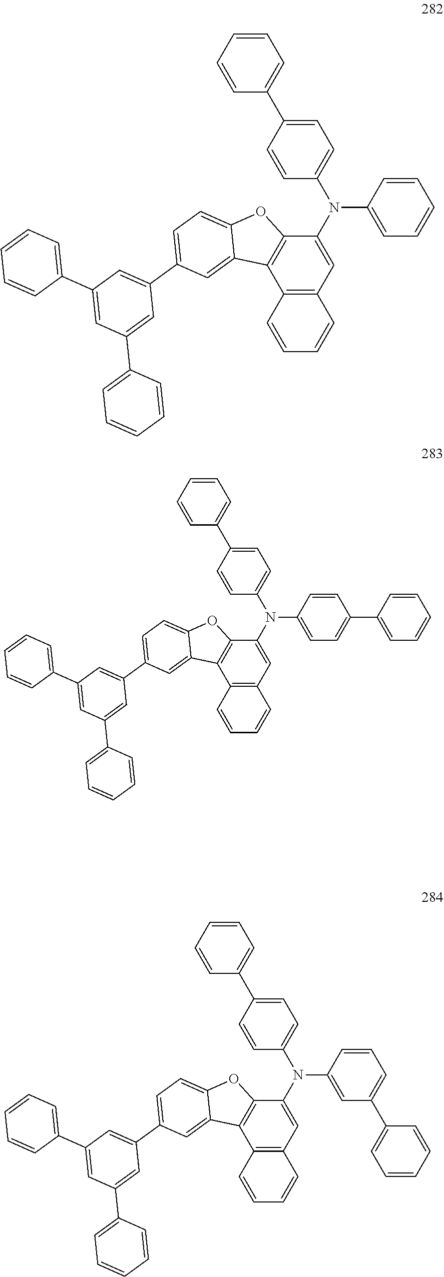 [SMILES: c1ccc(-c2ccc(N(c3ccccc3)c3cc4ccccc4c4c3oc3ccc(-c5cc(-c6ccccc6)cc(-c6ccccc6)c5)cc34)cc2)cc1 ; c1ccc(-c2ccc(N(c3ccc(-c4ccccc4)cc3)c3cc4ccccc4c4c3oc3ccc(-c5cc(-c6ccccc6)cc(-c6ccccc6)c5)cc34)cc2)cc1 ; c1ccc(-c2ccc(N(c3cccc(-c4ccccc4)c3)c3cc4ccccc4c4c3oc3ccc(-c5cc(-c6ccccc6)cc(-c6ccccc6)c5)cc34)cc2)cc1]

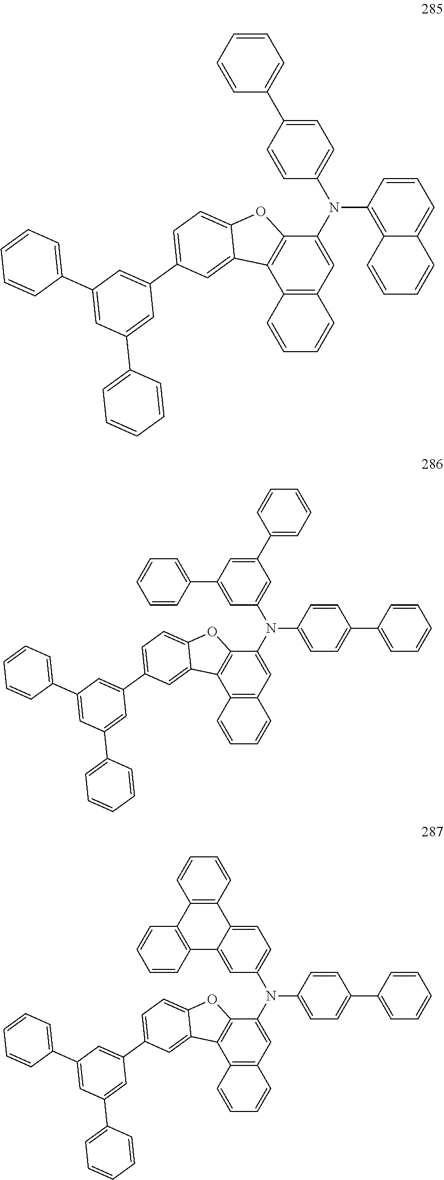 [SMILES: c1ccc(-c2ccc(N(c3cccc4ccccc34)c3cc4ccccc4c4c3oc3ccc(-c5cc(-c6ccccc6)cc(-c6ccccc6)c5)cc34)cc2)cc1 ; c1ccc(-c2ccc(N(c3cc(-c4ccccc4)cc(-c4ccccc4)c3)c3cc4ccccc4c4c3oc3ccc(-c5cc(-c6ccccc6)cc(-c6ccccc6)c5)cc34)cc2)cc1 ; c1ccc(-c2ccc(N(c3ccc4c5ccccc5c5ccccc5c4c3)c3cc4ccccc4c4c3oc3ccc(-c5cc(-c6ccccc6)cc(-c6ccccc6)c5)cc34)cc2)cc1]

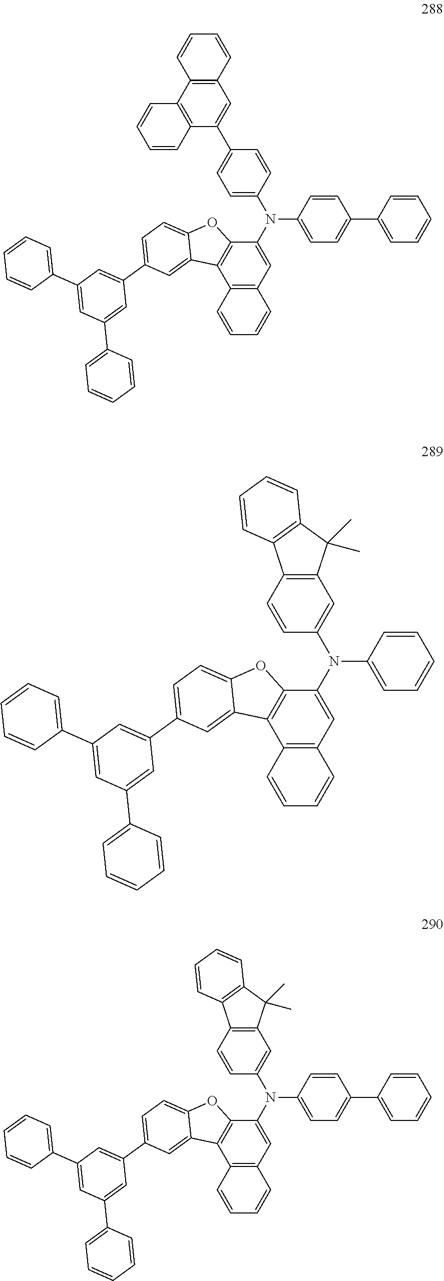 [SMILES: c1ccc(-c2ccc(N(c3ccc(-c4cc5ccccc5c5ccccc45)cc3)c3cc4ccccc4c4c3oc3ccc(-c5cc(-c6ccccc6)cc(-c6ccccc6)c5)cc34)cc2)cc1 ; CC1(C)c2ccccc2-c2ccc(N(c3ccccc3)c3cc4ccccc4c4c3oc3ccc(-c5cc(-c6ccccc6)cc(-c6ccccc6)c5)cc34)cc21 ; CC1(C)c2ccccc2-c2ccc(N(c3ccc(-c4ccccc4)cc3)c3cc4ccccc4c4c3oc3ccc(-c5cc(-c6ccccc6)cc(-c6ccccc6)c5)cc34)cc21]

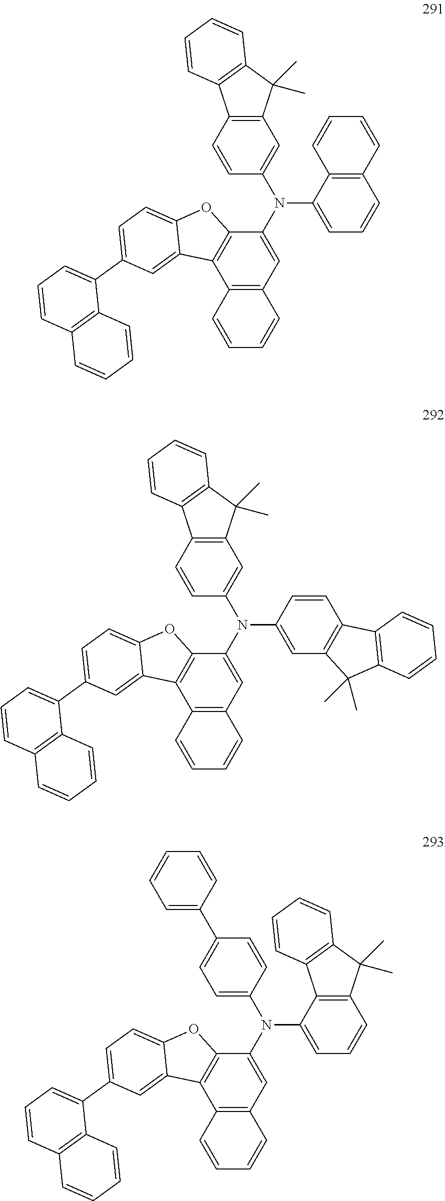 [SMILES: CC1(C)c2ccccc2-c2ccc(N(c3cccc4ccccc34)c3cc4ccccc4c4c3oc3ccc(-c5cccc6ccccc56)cc34)cc21 ; CC1(C)c2ccccc2-c2ccc(N(c3ccc4c(c3)C(C)(C)c3ccccc3-4)c3cc4ccccc4c4c3oc3ccc(-c5cccc6ccccc56)cc34)cc21 ; CC1(C)c2ccccc2-c2c(N(c3ccc(-c4ccccc4)cc3)c3cc4ccccc4c4c3oc3ccc(-c5cccc6ccccc56)cc34)cccc21]

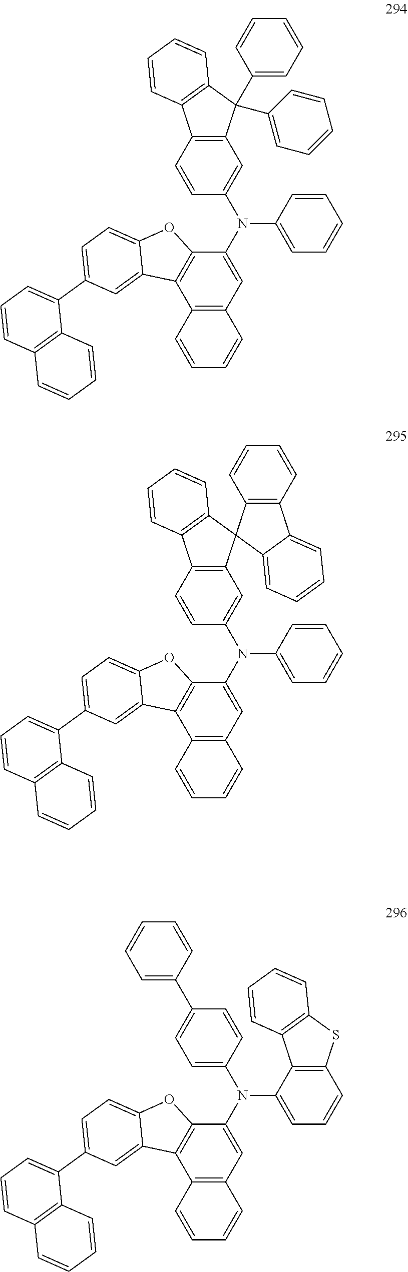 [SMILES: c1ccc(N(c2ccc3c(c2)C(c2ccccc2)(c2ccccc2)c2ccccc2-3)c2cc3ccccc3c3c2oc2ccc(-c4cccc5ccccc45)cc23)cc1 ; c1ccc(N(c2ccc3c(c2)C2(c4ccccc4-c4ccccc42)c2ccccc2-3)c2cc3ccccc3c3c2oc2ccc(-c4cccc5ccccc45)cc23)cc1 ; c1ccc(-c2ccc(N(c3cc4ccccc4c4c3oc3ccc(-c5cccc6ccccc56)cc34)c3cccc4sc5ccccc5c34)cc2)cc1]

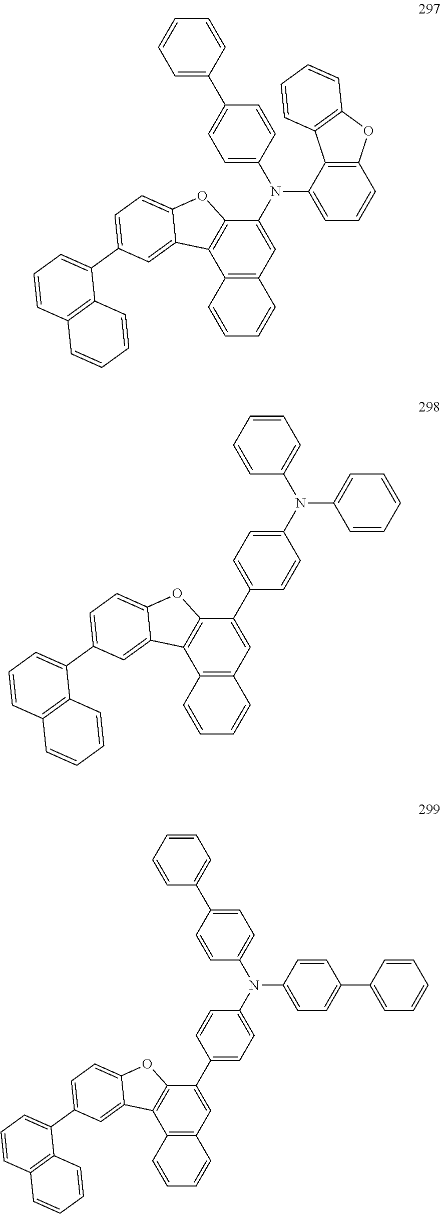 [SMILES: c1ccc(-c2ccc(N(c3cc4ccccc4c4c3oc3ccc(-c5cccc6ccccc56)cc34)c3cccc4oc5ccccc5c34)cc2)cc1 ; c1ccc(N(c2ccccc2)c2ccc(-c3cc4ccccc4c4c3oc3ccc(-c5cccc6ccccc56)cc34)cc2)cc1 ; c1ccc(-c2ccc(N(c3ccc(-c4ccccc4)cc3)c3ccc(-c4cc5ccccc5c5c4oc4ccc(-c6cccc7ccccc67)cc45)cc3)cc2)cc1]

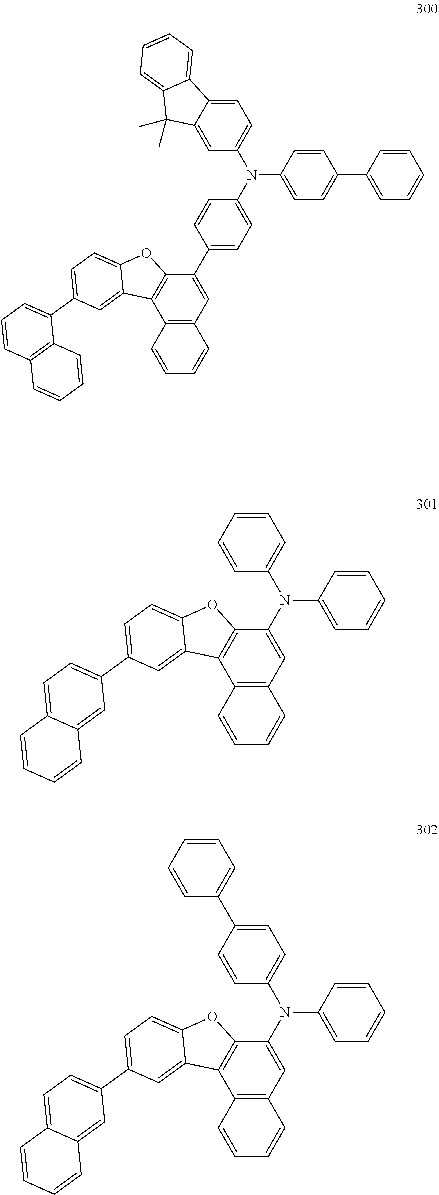 [SMILES: CC1(C)c2ccccc2-c2ccc(N(c3ccc(-c4ccccc4)cc3)c3ccc(-c4cc5ccccc5c5c4oc4ccc(-c6cccc7ccccc67)cc45)cc3)cc21 ; c1ccc(N(c2ccccc2)c2cc3ccccc3c3c2oc2ccc(-c4ccc5ccccc5c4)cc23)cc1 ; c1ccc(-c2ccc(N(c3ccccc3)c3cc4ccccc4c4c3oc3ccc(-c5ccc6ccccc6c5)cc34)cc2)cc1]

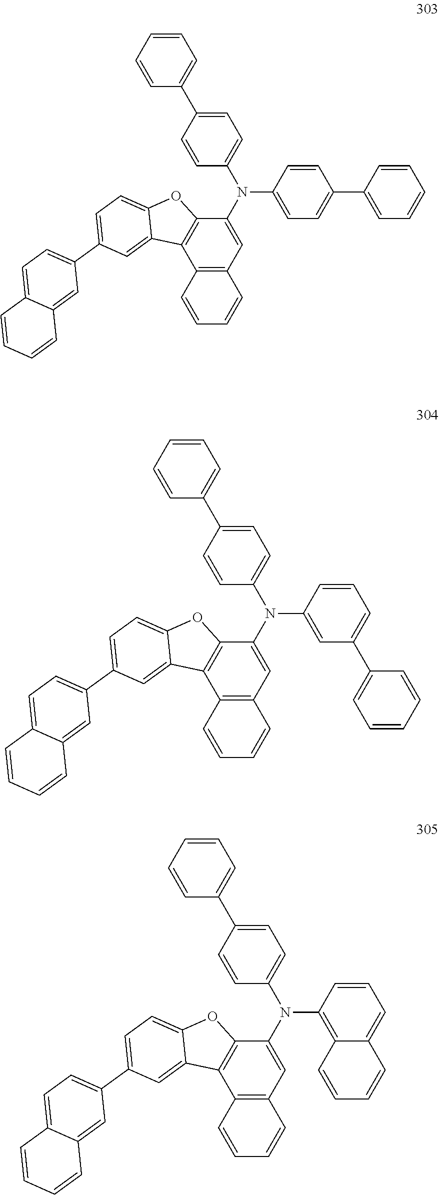 [SMILES: c1ccc(-c2ccc(N(c3ccc(-c4ccccc4)cc3)c3cc4ccccc4c4c3oc3ccc(-c5ccc6ccccc6c5)cc34)cc2)cc1 ; c1ccc(-c2ccc(N(c3cccc(-c4ccccc4)c3)c3cc4ccccc4c4c3oc3ccc(-c5ccc6ccccc6c5)cc34)cc2)cc1 ; c1ccc(-c2ccc(N(c3cccc4ccccc34)c3cc4ccccc4c4c3oc3ccc(-c5ccc6ccccc6c5)cc34)cc2)cc1]

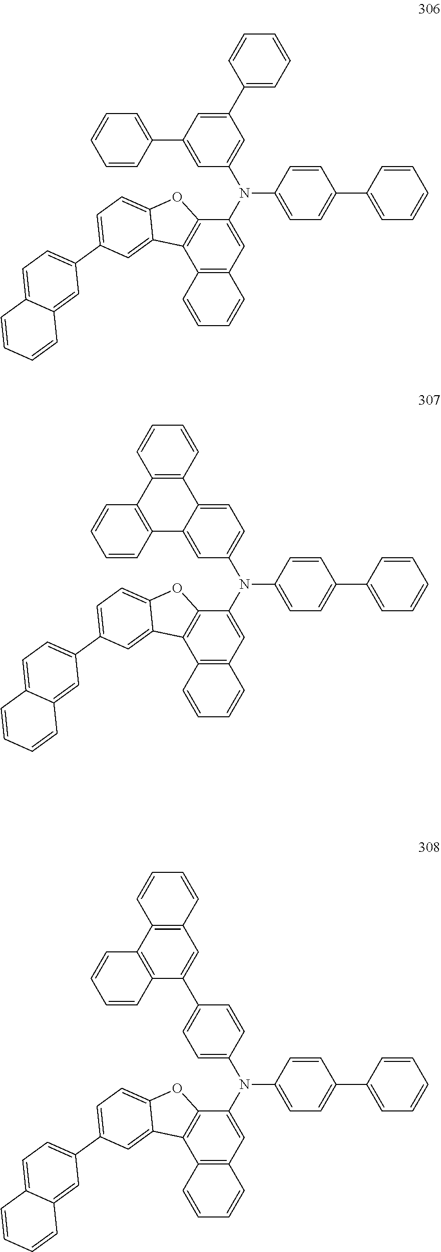 [SMILES: c1ccc(-c2ccc(N(c3cc(-c4ccccc4)cc(-c4ccccc4)c3)c3cc4ccccc4c4c3oc3ccc(-c5ccc6ccccc6c5)cc34)cc2)cc1 ; c1ccc(-c2ccc(N(c3ccc4c5ccccc5c5ccccc5c4c3)c3cc4ccccc4c4c3oc3ccc(-c5ccc6ccccc6c5)cc34)cc2)cc1 ; c1ccc(-c2ccc(N(c3ccc(-c4cc5ccccc5c5ccccc45)cc3)c3cc4ccccc4c4c3oc3ccc(-c5ccc6ccccc6c5)cc34)cc2)cc1]

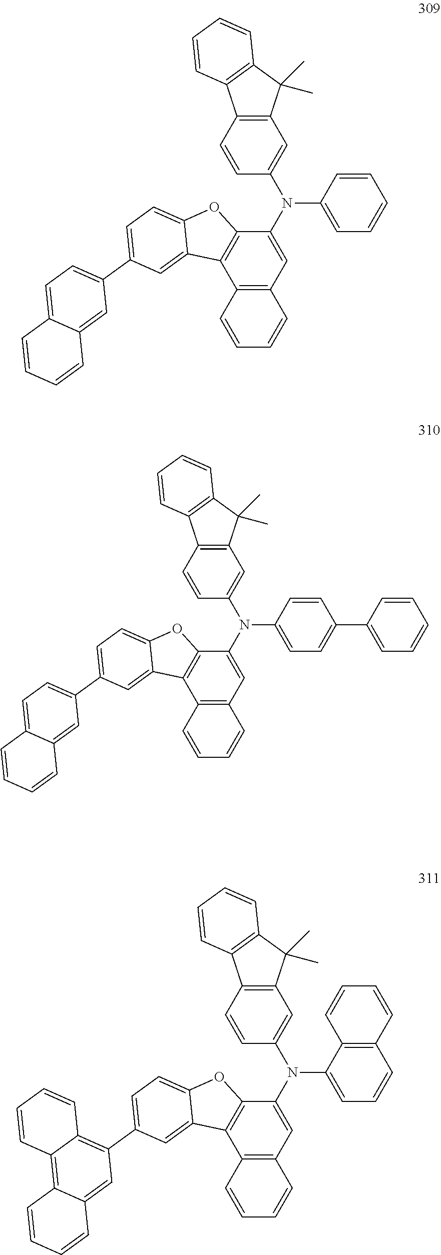 [SMILES: CC1(C)c2ccccc2-c2ccc(N(c3ccccc3)c3cc4ccccc4c4c3oc3ccc(-c5ccc6ccccc6c5)cc34)cc21 ; CC1(C)c2ccccc2-c2ccc(N(c3ccc(-c4ccccc4)cc3)c3cc4ccccc4c4c3oc3ccc(-c5ccc6ccccc6c5)cc34)cc21 ; CC1(C)c2ccccc2-c2ccc(N(c3cccc4ccccc34)c3cc4ccccc4c4c3oc3ccc(-c5cc6ccccc6c6ccccc56)cc34)cc21]

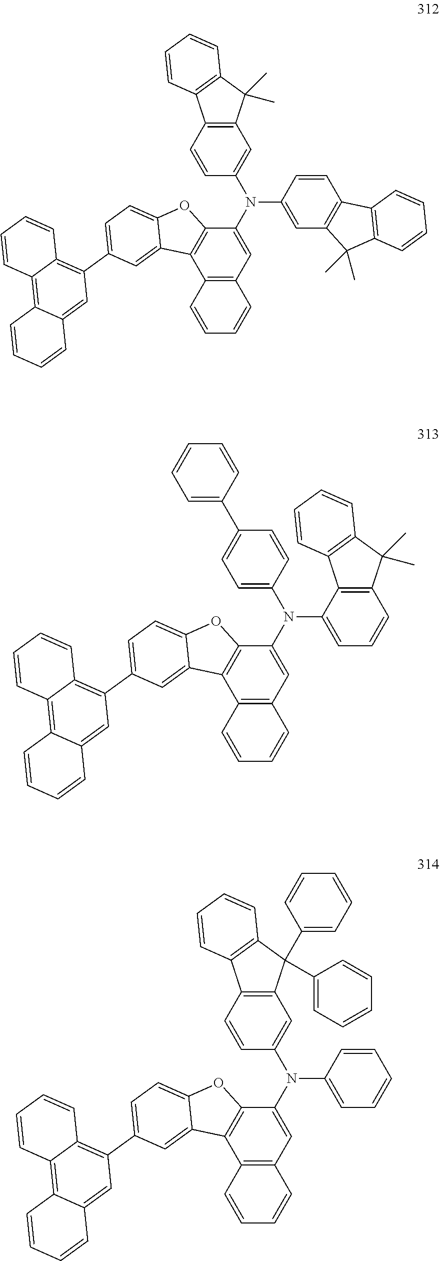 [SMILES: CC1(C)c2ccccc2-c2ccc(N(c3ccc4c(c3)C(C)(C)c3ccccc3-4)c3cc4ccccc4c4c3oc3ccc(-c5cc6ccccc6c6ccccc56)cc34)cc21 ; CC1(C)c2ccccc2-c2c(N(c3ccc(-c4ccccc4)cc3)c3cc4ccccc4c4c3oc3ccc(-c5cc6ccccc6c6ccccc56)cc34)cccc21 ; c1ccc(N(c2ccc3c(c2)C(c2ccccc2)(c2ccccc2)c2ccccc2-3)c2cc3ccccc3c3c2oc2ccc(-c4cc5ccccc5c5ccccc45)cc23)cc1]

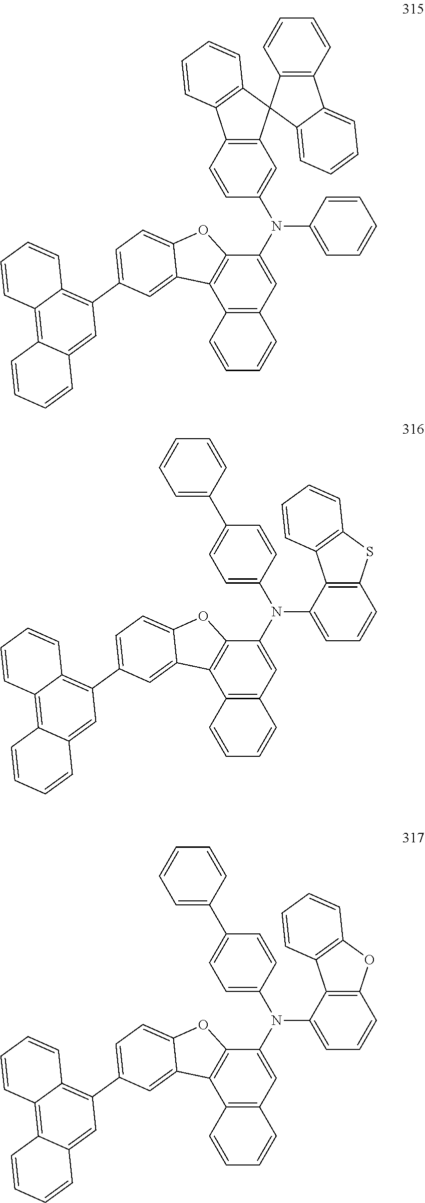 [SMILES: c1ccc(N(c2ccc3c(c2)C2(c4ccccc4-c4ccccc42)c2ccccc2-3)c2cc3ccccc3c3c2oc2ccc(-c4cc5ccccc5c5ccccc45)cc23)cc1 ; c1ccc(-c2ccc(N(c3cc4ccccc4c4c3oc3ccc(-c5cc6ccccc6c6ccccc56)cc34)c3cccc4sc5ccccc5c34)cc2)cc1 ; c1ccc(-c2ccc(N(c3cc4ccccc4c4c3oc3ccc(-c5cc6ccccc6c6ccccc56)cc34)c3cccc4oc5ccccc5c34)cc2)cc1]

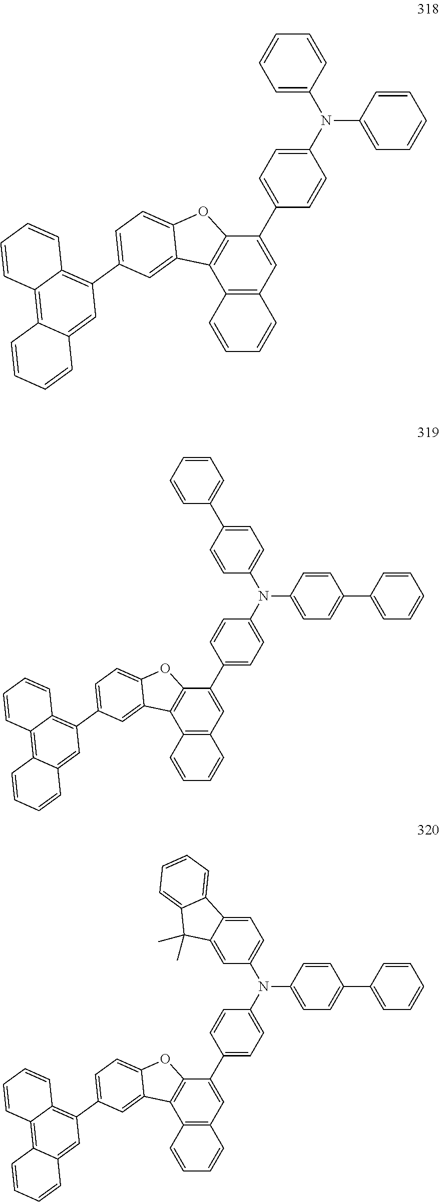 [SMILES: c1ccc(N(c2ccccc2)c2ccc(-c3cc4ccccc4c4c3oc3ccc(-c5cc6ccccc6c6ccccc56)cc34)cc2)cc1 ; c1ccc(-c2ccc(N(c3ccc(-c4ccccc4)cc3)c3ccc(-c4cc5ccccc5c5c4oc4ccc(-c6cc7ccccc7c7ccccc67)cc45)cc3)cc2)cc1 ; CC1(C)c2ccccc2-c2ccc(N(c3ccc(-c4ccccc4)cc3)c3ccc(-c4cc5ccccc5c5c4oc4ccc(-c6cc7ccccc7c7ccccc67)cc45)cc3)cc21]

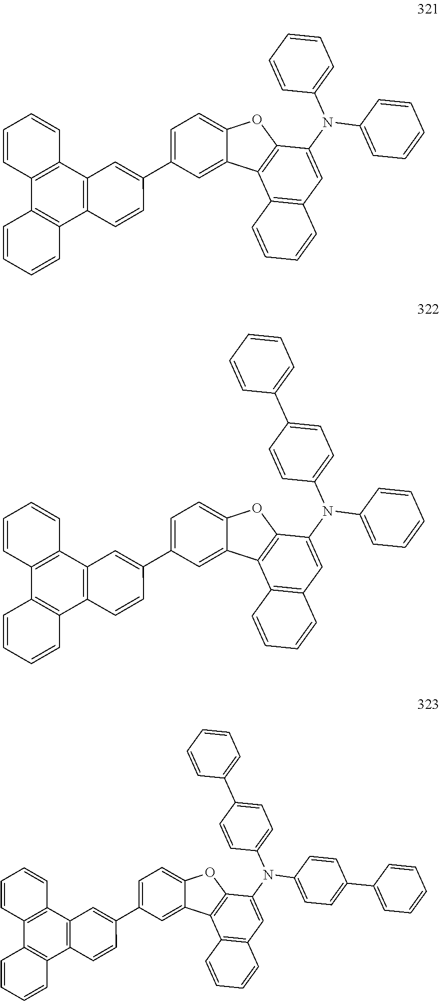 [SMILES: c1ccc(N(c2ccccc2)c2cc3ccccc3c3c2oc2ccc(-c4ccc5c6ccccc6c6ccccc6c5c4)cc23)cc1 ; c1ccc(-c2ccc(N(c3ccccc3)c3cc4ccccc4c4c3oc3ccc(-c5ccc6c7ccccc7c7ccccc7c6c5)cc34)cc2)cc1 ; c1ccc(-c2ccc(N(c3ccc(-c4ccccc4)cc3)c3cc4ccccc4c4c3oc3ccc(-c5ccc6c7ccccc7c7ccccc7c6c5)cc34)cc2)cc1]

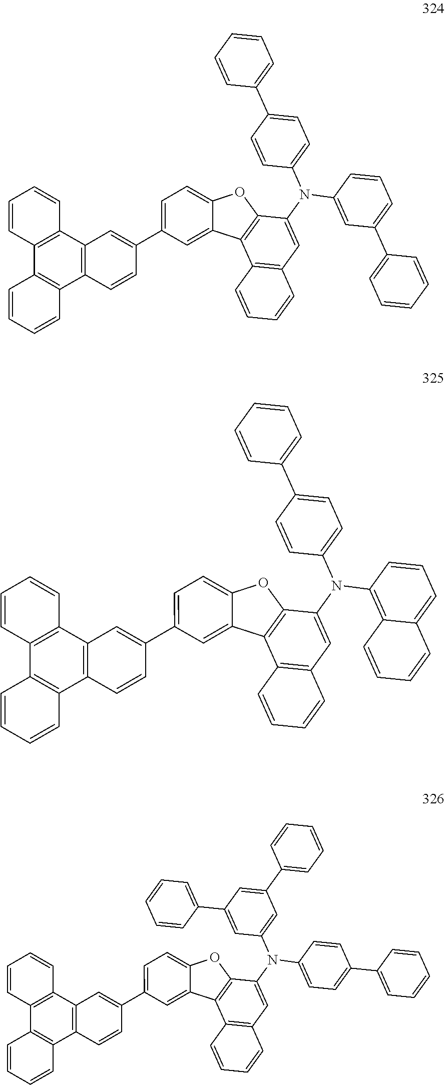 [SMILES: c1ccc(-c2ccc(N(c3cccc(-c4ccccc4)c3)c3cc4ccccc4c4c3oc3ccc(-c5ccc6c7ccccc7c7ccccc7c6c5)cc34)cc2)cc1 ; c1ccc(-c2ccc(N(c3cccc4ccccc34)c3cc4ccccc4c4c3oc3ccc(-c5ccc6c7ccccc7c7ccccc7c6c5)cc34)cc2)cc1 ; c1ccc(-c2ccc(N(c3cc(-c4ccccc4)cc(-c4ccccc4)c3)c3cc4ccccc4c4c3oc3ccc(-c5ccc6c7ccccc7c7ccccc7c6c5)cc34)cc2)cc1]

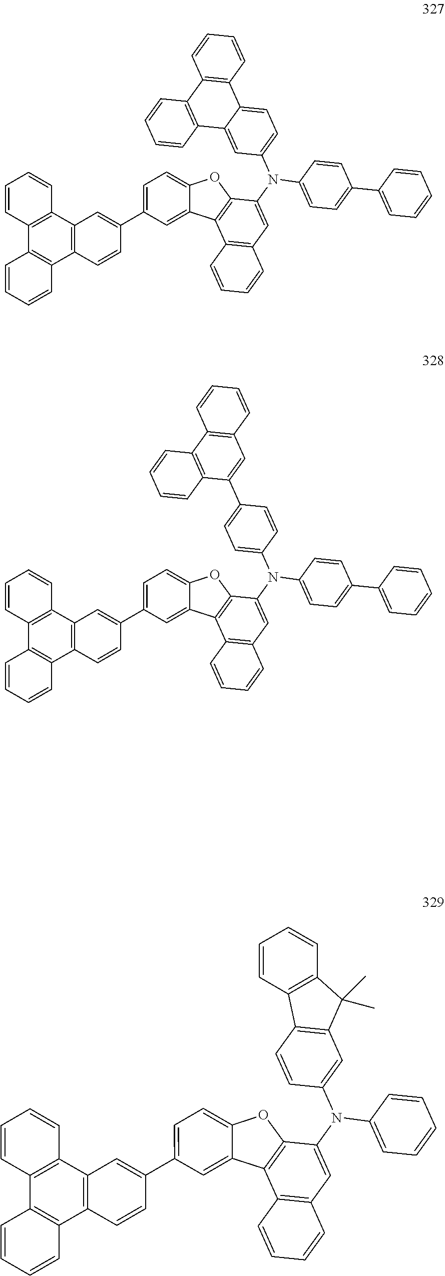 [SMILES: c1ccc(-c2ccc(N(c3ccc4c5ccccc5c5ccccc5c4c3)c3cc4ccccc4c4c3oc3ccc(-c5ccc6c7ccccc7c7ccccc7c6c5)cc34)cc2)cc1 ; c1ccc(-c2ccc(N(c3ccc(-c4cc5ccccc5c5ccccc45)cc3)c3cc4ccccc4c4c3oc3ccc(-c5ccc6c7ccccc7c7ccccc7c6c5)cc34)cc2)cc1 ; CC1(C)c2ccccc2-c2ccc(N(c3ccccc3)c3cc4ccccc4c4c3oc3ccc(-c5ccc6c7ccccc7c7ccccc7c6c5)cc34)cc21]

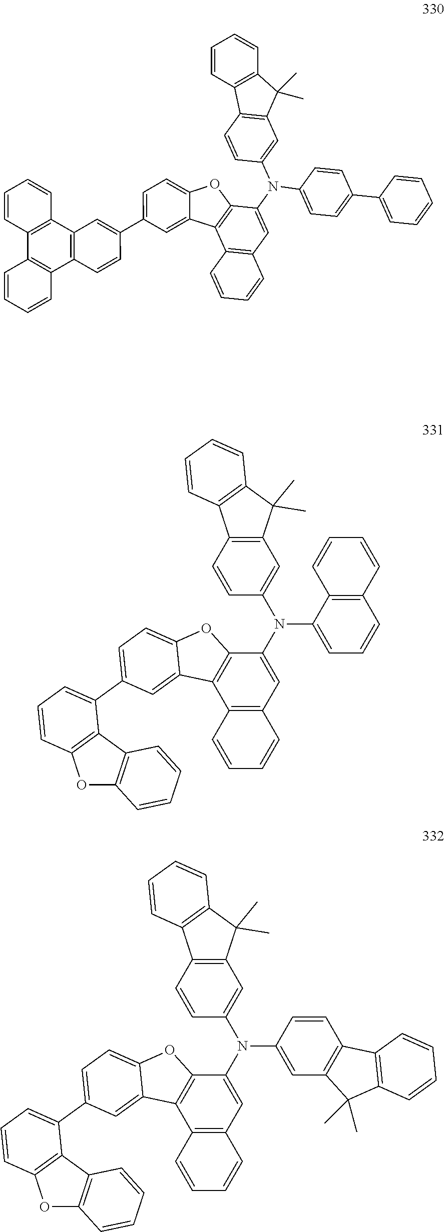 [SMILES: CC1(C)c2ccccc2-c2ccc(N(c3ccc(-c4ccccc4)cc3)c3cc4ccccc4c4c3oc3ccc(-c5ccc6c7ccccc7c7ccccc7c6c5)cc34)cc21 ; CC1(C)c2ccccc2-c2ccc(N(c3cccc4ccccc34)c3cc4ccccc4c4c3oc3ccc(-c5cccc6oc7ccccc7c56)cc34)cc21 ; CC1(C)c2ccccc2-c2ccc(N(c3ccc4c(c3)C(C)(C)c3ccccc3-4)c3cc4ccccc4c4c3oc3ccc(-c5cccc6oc7ccccc7c56)cc34)cc21]

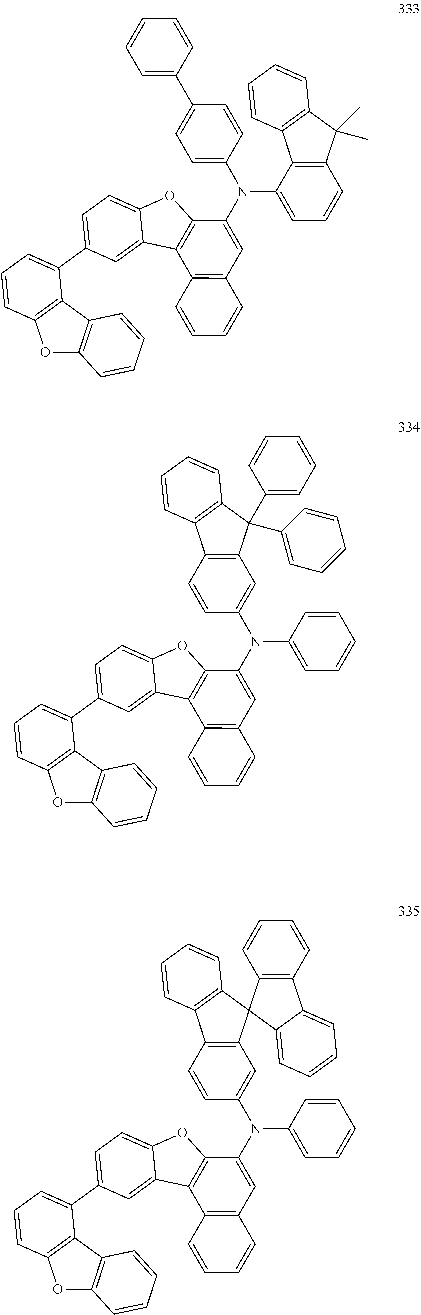 [SMILES: CC1(C)c2ccccc2-c2c(N(c3ccc(-c4ccccc4)cc3)c3cc4ccccc4c4c3oc3ccc(-c5cccc6oc7ccccc7c56)cc34)cccc21 ; c1ccc(N(c2ccc3c(c2)C(c2ccccc2)(c2ccccc2)c2ccccc2-3)c2cc3ccccc3c3c2oc2ccc(-c4cccc5oc6ccccc6c45)cc23)cc1 ; c1ccc(N(c2ccc3c(c2)C2(c4ccccc4-c4ccccc42)c2ccccc2-3)c2cc3ccccc3c3c2oc2ccc(-c4cccc5oc6ccccc6c45)cc23)cc1]

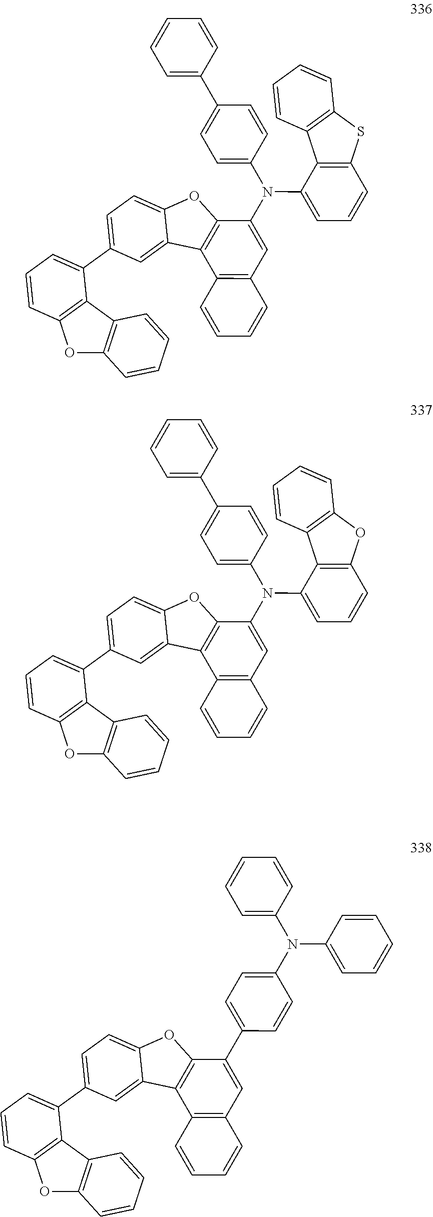 [SMILES: c1ccc(-c2ccc(N(c3cc4ccccc4c4c3oc3ccc(-c5cccc6oc7ccccc7c56)cc34)c3cccc4sc5ccccc5c34)cc2)cc1 ; c1ccc(-c2ccc(N(c3cc4ccccc4c4c3oc3ccc(-c5cccc6oc7ccccc7c56)cc34)c3cccc4oc5ccccc5c34)cc2)cc1 ; c1ccc(N(c2ccccc2)c2ccc(-c3cc4ccccc4c4c3oc3ccc(-c5cccc6oc7ccccc7c56)cc34)cc2)cc1]

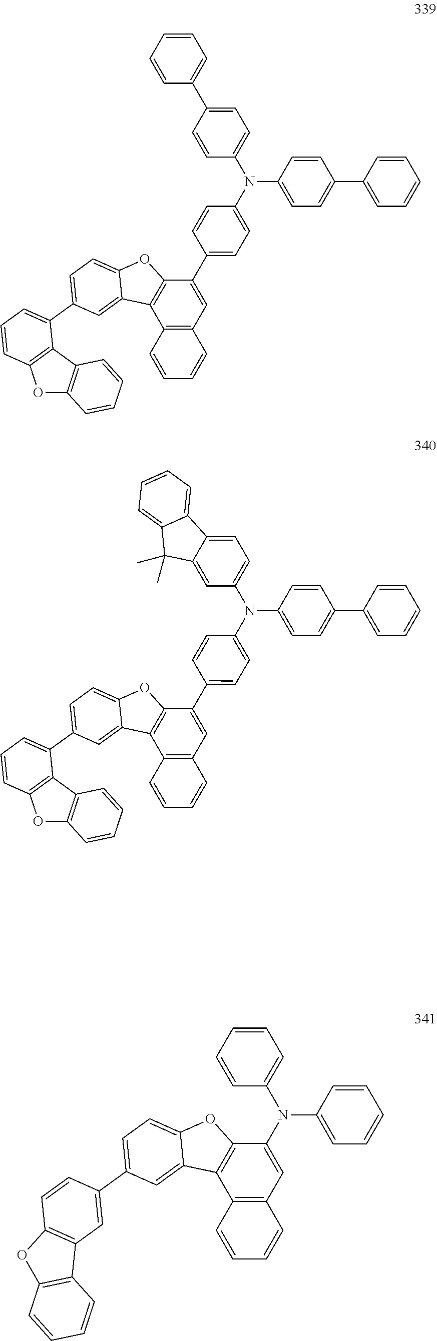 [SMILES: c1ccc(-c2ccc(N(c3ccc(-c4ccccc4)cc3)c3ccc(-c4cc5ccccc5c5c4oc4ccc(-c6cccc7oc8ccccc8c67)cc45)cc3)cc2)cc1 ; CC1(C)c2ccccc2-c2ccc(N(c3ccc(-c4ccccc4)cc3)c3ccc(-c4cc5ccccc5c5c4oc4ccc(-c6cccc7oc8ccccc8c67)cc45)cc3)cc21 ; c1ccc(N(c2ccccc2)c2cc3ccccc3c3c2oc2ccc(-c4ccc5oc6ccccc6c5c4)cc23)cc1]

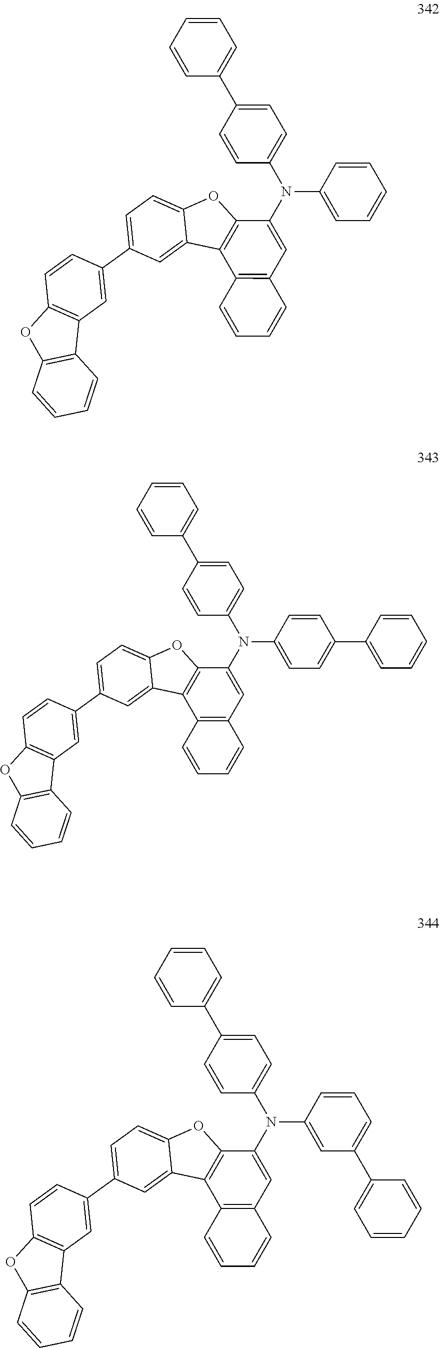 [SMILES: c1ccc(-c2ccc(N(c3ccccc3)c3cc4ccccc4c4c3oc3ccc(-c5ccc6oc7ccccc7c6c5)cc34)cc2)cc1 ; c1ccc(-c2ccc(N(c3ccc(-c4ccccc4)cc3)c3cc4ccccc4c4c3oc3ccc(-c5ccc6oc7ccccc7c6c5)cc34)cc2)cc1 ; c1ccc(-c2ccc(N(c3cccc(-c4ccccc4)c3)c3cc4ccccc4c4c3oc3ccc(-c5ccc6oc7ccccc7c6c5)cc34)cc2)cc1]

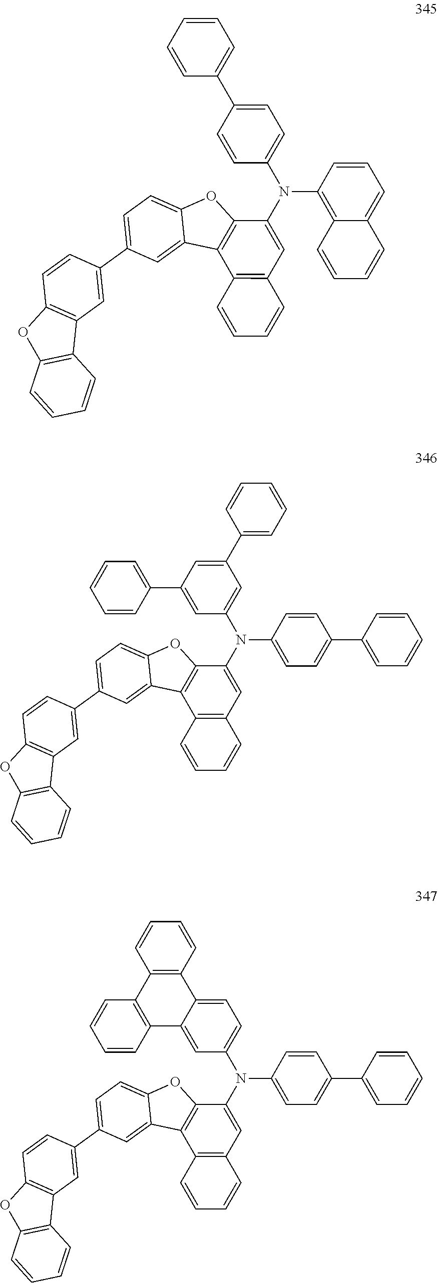 [SMILES: c1ccc(-c2ccc(N(c3cccc4ccccc34)c3cc4ccccc4c4c3oc3ccc(-c5ccc6oc7ccccc7c6c5)cc34)cc2)cc1 ; c1ccc(-c2ccc(N(c3cc(-c4ccccc4)cc(-c4ccccc4)c3)c3cc4ccccc4c4c3oc3ccc(-c5ccc6oc7ccccc7c6c5)cc34)cc2)cc1 ; c1ccc(-c2ccc(N(c3ccc4c5ccccc5c5ccccc5c4c3)c3cc4ccccc4c4c3oc3ccc(-c5ccc6oc7ccccc7c6c5)cc34)cc2)cc1]

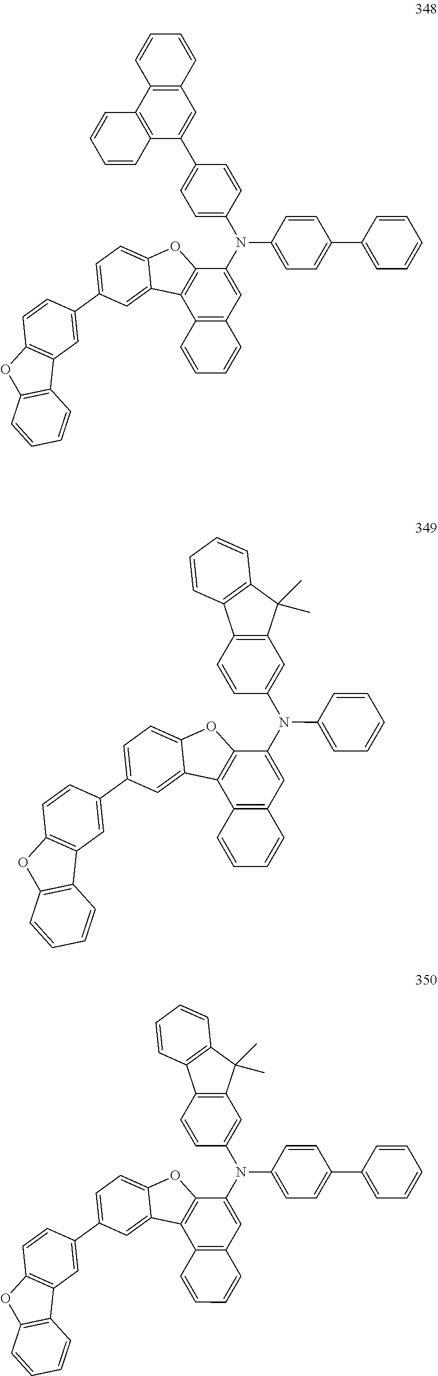 [SMILES: c1ccc(-c2ccc(N(c3ccc(-c4cc5ccccc5c5ccccc45)cc3)c3cc4ccccc4c4c3oc3ccc(-c5ccc6oc7ccccc7c6c5)cc34)cc2)cc1 ; CC1(C)c2ccccc2-c2ccc(N(c3ccccc3)c3cc4ccccc4c4c3oc3ccc(-c5ccc6oc7ccccc7c6c5)cc34)cc21 ; CC1(C)c2ccccc2-c2ccc(N(c3ccc(-c4ccccc4)cc3)c3cc4ccccc4c4c3oc3ccc(-c5ccc6oc7ccccc7c6c5)cc34)cc21]

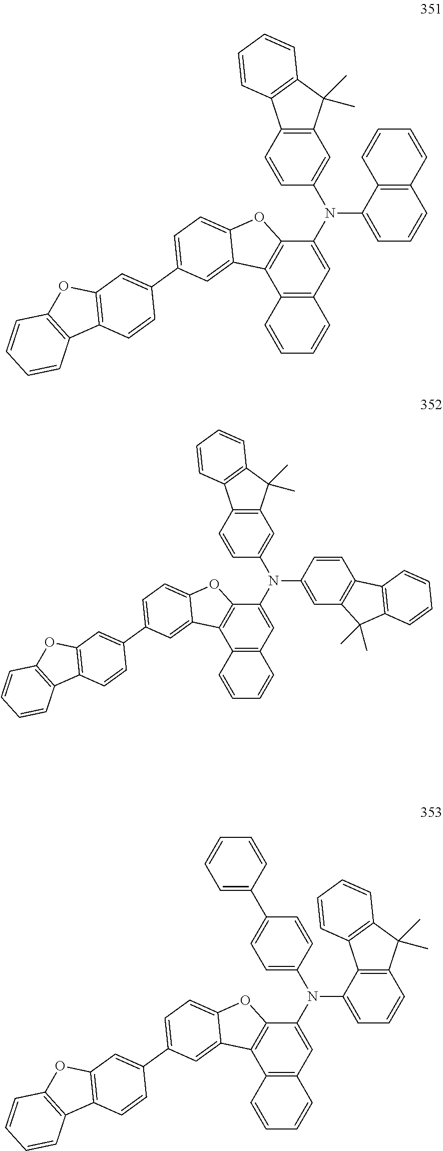 [SMILES: CC1(C)c2ccccc2-c2ccc(N(c3cccc4ccccc34)c3cc4ccccc4c4c3oc3ccc(-c5ccc6c(c5)oc5ccccc56)cc34)cc21 ; CC1(C)c2ccccc2-c2ccc(N(c3ccc4c(c3)C(C)(C)c3ccccc3-4)c3cc4ccccc4c4c3oc3ccc(-c5ccc6c(c5)oc5ccccc56)cc34)cc21 ; CC1(C)c2ccccc2-c2c(N(c3ccc(-c4ccccc4)cc3)c3cc4ccccc4c4c3oc3ccc(-c5ccc6c(c5)oc5ccccc56)cc34)cccc21]

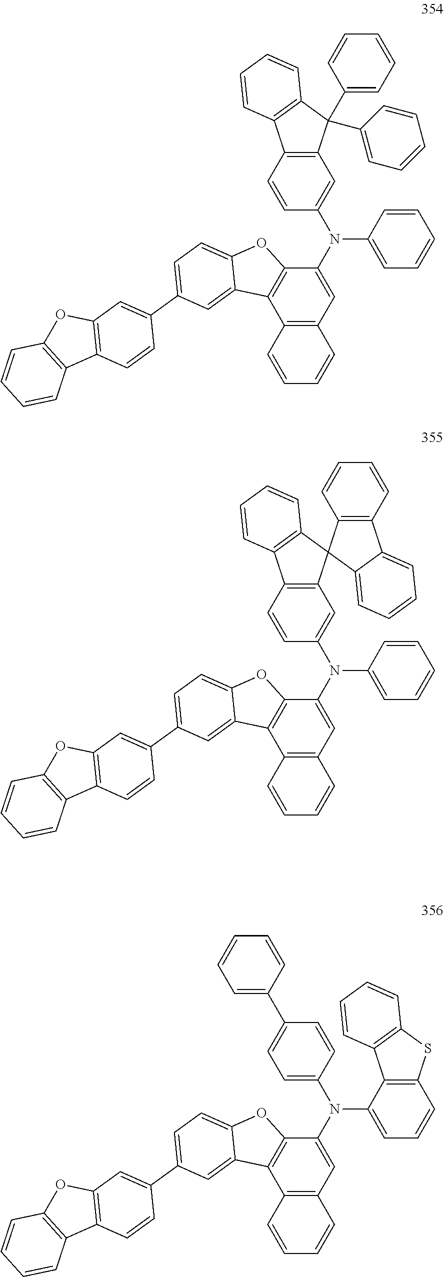 [SMILES: c1ccc(N(c2ccc3c(c2)C(c2ccccc2)(c2ccccc2)c2ccccc2-3)c2cc3ccccc3c3c2oc2ccc(-c4ccc5c(c4)oc4ccccc45)cc23)cc1 ; c1ccc(N(c2ccc3c(c2)C2(c4ccccc4-c4ccccc42)c2ccccc2-3)c2cc3ccccc3c3c2oc2ccc(-c4ccc5c(c4)oc4ccccc45)cc23)cc1 ; c1ccc(-c2ccc(N(c3cc4ccccc4c4c3oc3ccc(-c5ccc6c(c5)oc5ccccc56)cc34)c3cccc4sc5ccccc5c34)cc2)cc1]

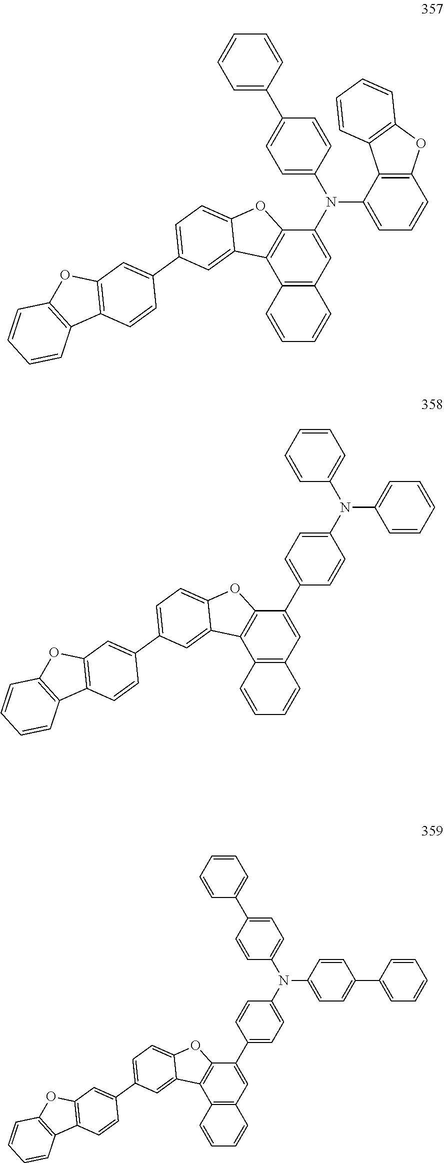 [SMILES: c1ccc(-c2ccc(N(c3cc4ccccc4c4c3oc3ccc(-c5ccc6c(c5)oc5ccccc56)cc34)c3cccc4oc5ccccc5c34)cc2)cc1 ; c1ccc(N(c2ccccc2)c2ccc(-c3cc4ccccc4c4c3oc3ccc(-c5ccc6c(c5)oc5ccccc56)cc34)cc2)cc1 ; c1ccc(-c2ccc(N(c3ccc(-c4ccccc4)cc3)c3ccc(-c4cc5ccccc5c5c4oc4ccc(-c6ccc7c(c6)oc6ccccc67)cc45)cc3)cc2)cc1]

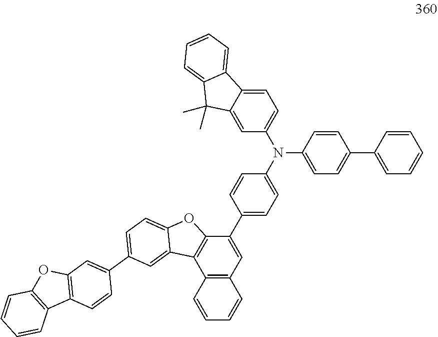 [SMILES: CC1(C)c2ccccc2-c2ccc(N(c3ccc(-c4ccccc4)cc3)c3ccc(-c4cc5ccccc5c5c4oc4ccc(-c6ccc7c(c6)oc6ccccc67)cc45)cc3)cc21]

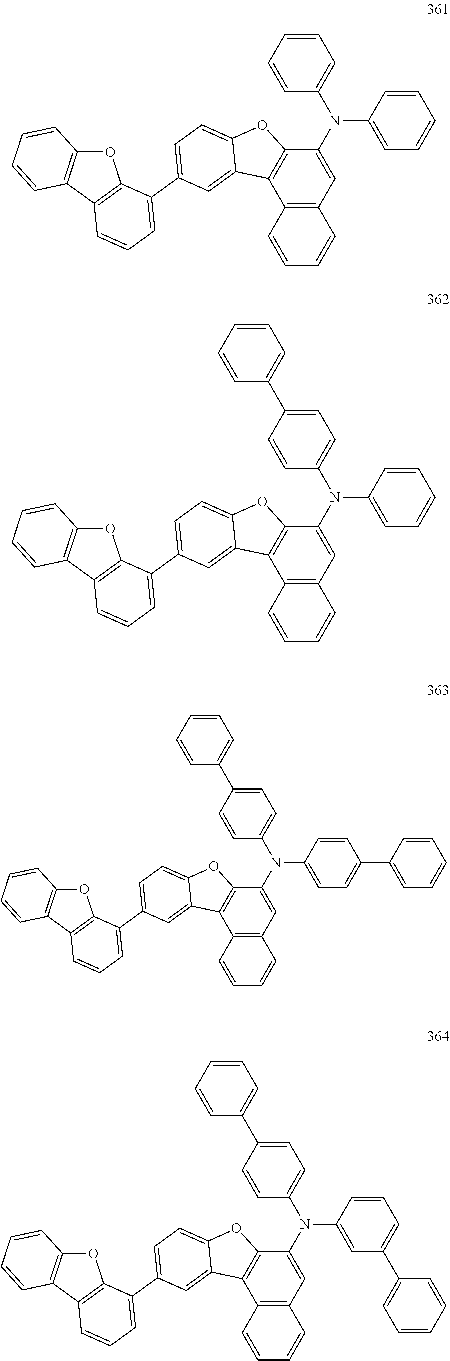 [SMILES: c1ccc(N(c2ccccc2)c2cc3ccccc3c3c2oc2ccc(-c4cccc5c4oc4ccccc45)cc23)cc1 ; c1ccc(-c2ccc(N(c3ccccc3)c3cc4ccccc4c4c3oc3ccc(-c5cccc6c5oc5ccccc56)cc34)cc2)cc1 ; c1ccc(-c2ccc(N(c3ccc(-c4ccccc4)cc3)c3cc4ccccc4c4c3oc3ccc(-c5cccc6c5oc5ccccc56)cc34)cc2)cc1 ; c1ccc(-c2ccc(N(c3cccc(-c4ccccc4)c3)c3cc4ccccc4c4c3oc3ccc(-c5cccc6c5oc5ccccc56)cc34)cc2)cc1]

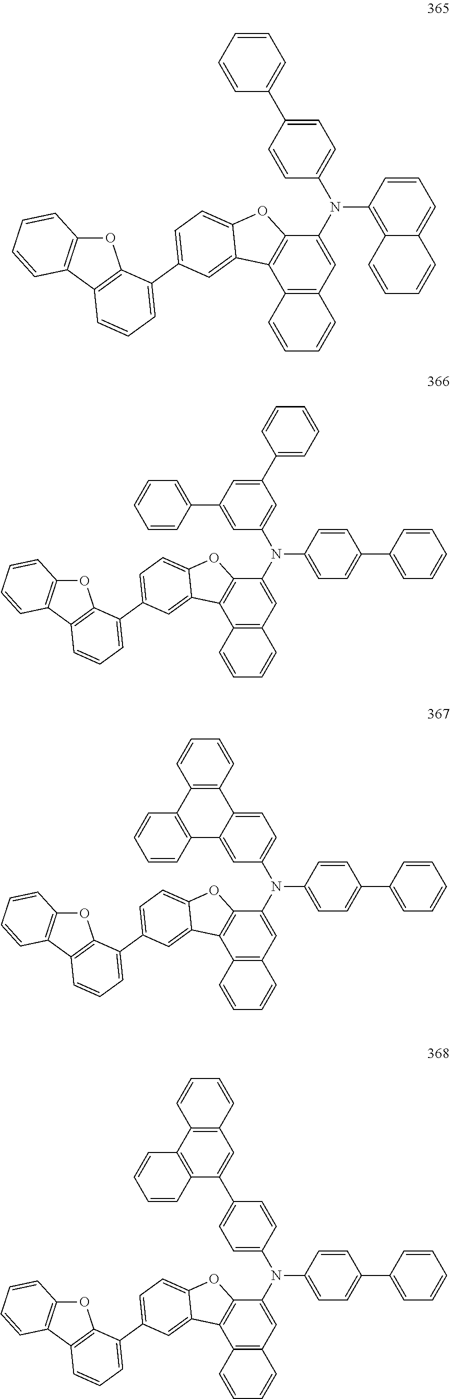 [SMILES: c1ccc(-c2ccc(N(c3cccc4ccccc34)c3cc4ccccc4c4c3oc3ccc(-c5cccc6c5oc5ccccc56)cc34)cc2)cc1 ; c1ccc(-c2ccc(N(c3cc(-c4ccccc4)cc(-c4ccccc4)c3)c3cc4ccccc4c4c3oc3ccc(-c5cccc6c5oc5ccccc56)cc34)cc2)cc1 ; c1ccc(-c2ccc(N(c3ccc4c5ccccc5c5ccccc5c4c3)c3cc4ccccc4c4c3oc3ccc(-c5cccc6c5oc5ccccc56)cc34)cc2)cc1 ; c1ccc(-c2ccc(N(c3ccc(-c4cc5ccccc5c5ccccc45)cc3)c3cc4ccccc4c4c3oc3ccc(-c5cccc6c5oc5ccccc56)cc34)cc2)cc1]

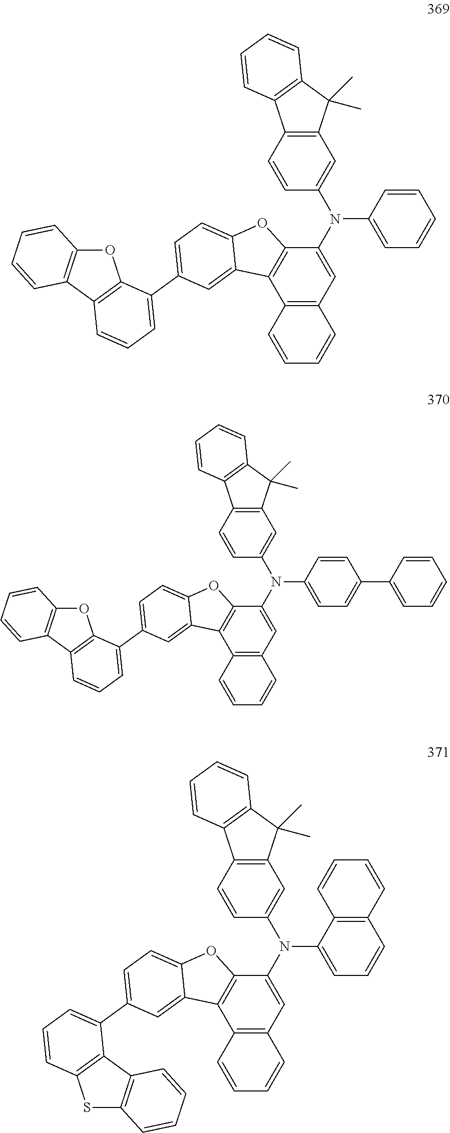 [SMILES: CC1(C)c2ccccc2-c2ccc(N(c3ccccc3)c3cc4ccccc4c4c3oc3ccc(-c5cccc6c5oc5ccccc56)cc34)cc21 ; CC1(C)c2ccccc2-c2ccc(N(c3ccc(-c4ccccc4)cc3)c3cc4ccccc4c4c3oc3ccc(-c5cccc6c5oc5ccccc56)cc34)cc21 ; CC1(C)c2ccccc2-c2ccc(N(c3cccc4ccccc34)c3cc4ccccc4c4c3oc3ccc(-c5cccc6sc7ccccc7c56)cc34)cc21]

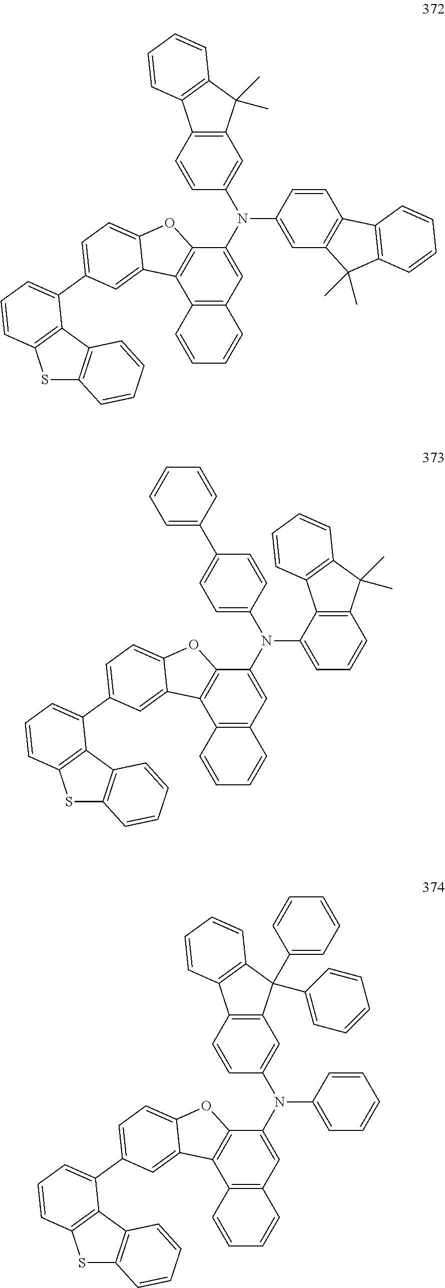 [SMILES: CC1(C)c2ccccc2-c2ccc(N(c3ccc4c(c3)C(C)(C)c3ccccc3-4)c3cc4ccccc4c4c3oc3ccc(-c5cccc6sc7ccccc7c56)cc34)cc21 ; CC1(C)c2ccccc2-c2c(N(c3ccc(-c4ccccc4)cc3)c3cc4ccccc4c4c3oc3ccc(-c5cccc6sc7ccccc7c56)cc34)cccc21 ; c1ccc(N(c2ccc3c(c2)C(c2ccccc2)(c2ccccc2)c2ccccc2-3)c2cc3ccccc3c3c2oc2ccc(-c4cccc5sc6ccccc6c45)cc23)cc1]

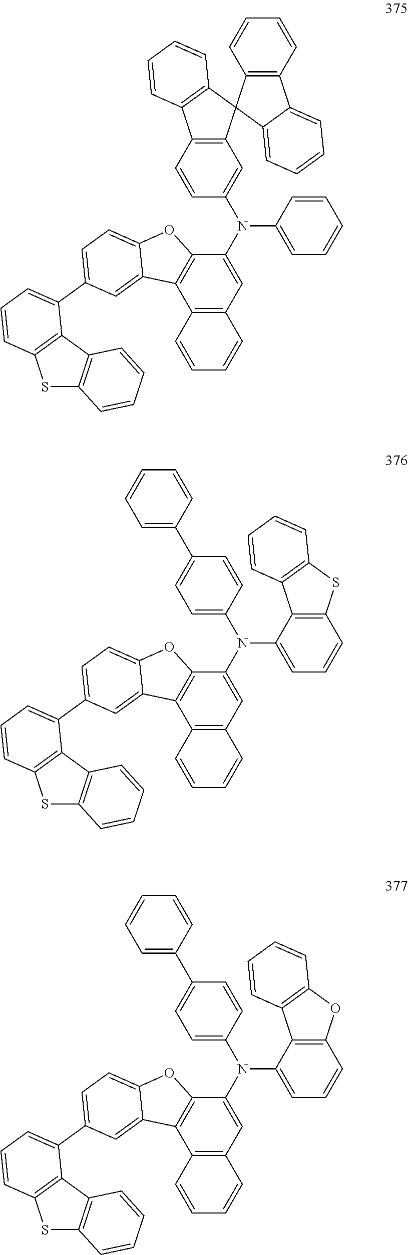 [SMILES: c1ccc(N(c2ccc3c(c2)C2(c4ccccc4-c4ccccc42)c2ccccc2-3)c2cc3ccccc3c3c2oc2ccc(-c4cccc5sc6ccccc6c45)cc23)cc1 ; c1ccc(-c2ccc(N(c3cc4ccccc4c4c3oc3ccc(-c5cccc6sc7ccccc7c56)cc34)c3cccc4sc5ccccc5c34)cc2)cc1 ; c1ccc(-c2ccc(N(c3cc4ccccc4c4c3oc3ccc(-c5cccc6sc7ccccc7c56)cc34)c3cccc4oc5ccccc5c34)cc2)cc1]

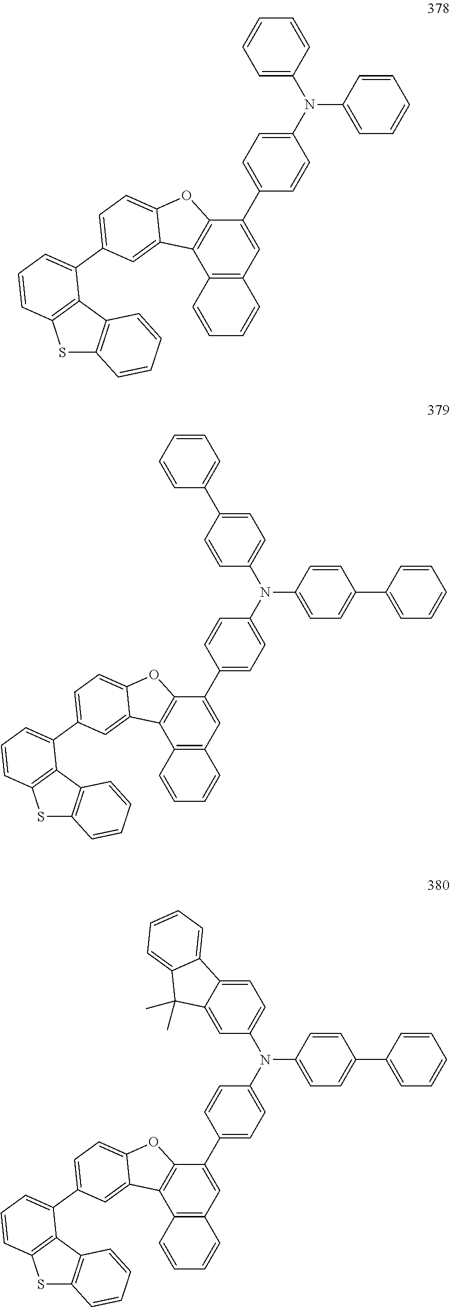 [SMILES: c1ccc(N(c2ccccc2)c2ccc(-c3cc4ccccc4c4c3oc3ccc(-c5cccc6sc7ccccc7c56)cc34)cc2)cc1 ; c1ccc(-c2ccc(N(c3ccc(-c4ccccc4)cc3)c3ccc(-c4cc5ccccc5c5c4oc4ccc(-c6cccc7sc8ccccc8c67)cc45)cc3)cc2)cc1 ; CC1(C)c2ccccc2-c2ccc(N(c3ccc(-c4ccccc4)cc3)c3ccc(-c4cc5ccccc5c5c4oc4ccc(-c6cccc7sc8ccccc8c67)cc45)cc3)cc21]

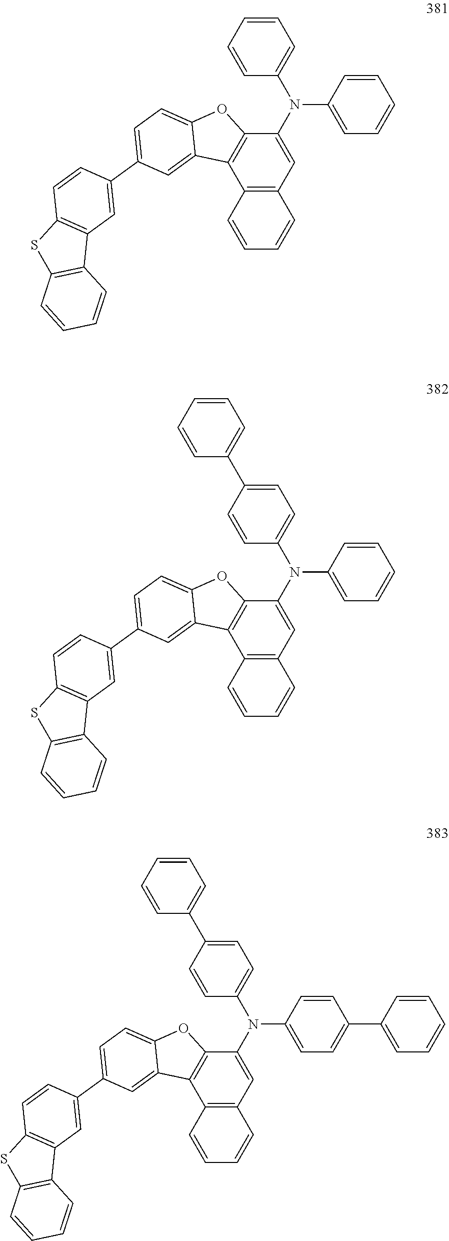 [SMILES: c1ccc(N(c2ccccc2)c2cc3ccccc3c3c2oc2ccc(-c4ccc5sc6ccccc6c5c4)cc23)cc1 ; c1ccc(-c2ccc(N(c3ccccc3)c3cc4ccccc4c4c3oc3ccc(-c5ccc6sc7ccccc7c6c5)cc34)cc2)cc1 ; c1ccc(-c2ccc(N(c3ccc(-c4ccccc4)cc3)c3cc4ccccc4c4c3oc3ccc(-c5ccc6sc7ccccc7c6c5)cc34)cc2)cc1]

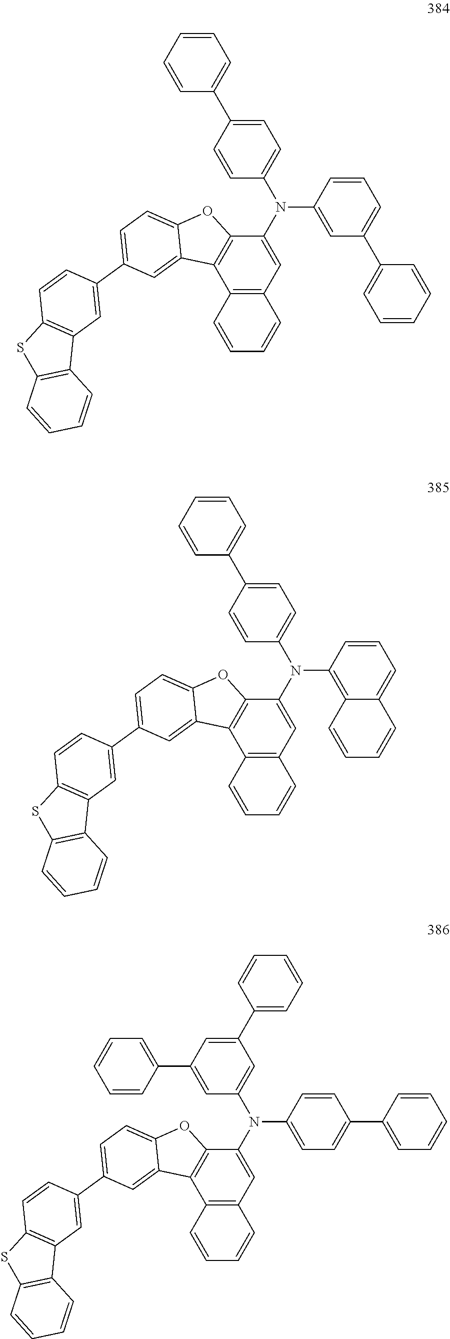 [SMILES: c1ccc(-c2ccc(N(c3cccc(-c4ccccc4)c3)c3cc4ccccc4c4c3oc3ccc(-c5ccc6sc7ccccc7c6c5)cc34)cc2)cc1 ; c1ccc(-c2ccc(N(c3cccc4ccccc34)c3cc4ccccc4c4c3oc3ccc(-c5ccc6sc7ccccc7c6c5)cc34)cc2)cc1 ; c1ccc(-c2ccc(N(c3cc(-c4ccccc4)cc(-c4ccccc4)c3)c3cc4ccccc4c4c3oc3ccc(-c5ccc6sc7ccccc7c6c5)cc34)cc2)cc1]

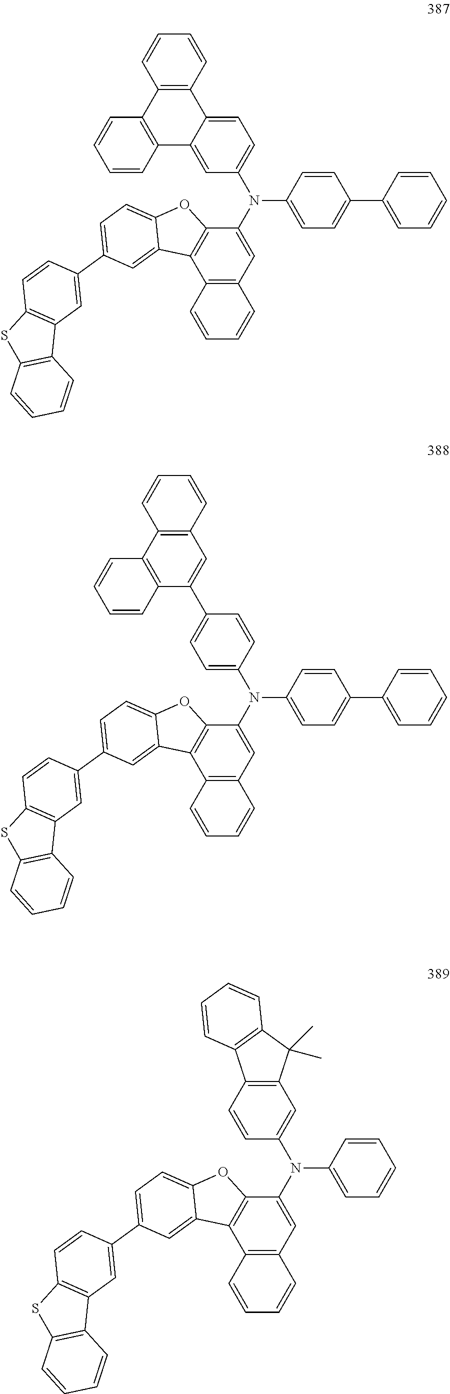 [SMILES: c1ccc(-c2ccc(N(c3ccc4c5ccccc5c5ccccc5c4c3)c3cc4ccccc4c4c3oc3ccc(-c5ccc6sc7ccccc7c6c5)cc34)cc2)cc1 ; c1ccc(-c2ccc(N(c3ccc(-c4cc5ccccc5c5ccccc45)cc3)c3cc4ccccc4c4c3oc3ccc(-c5ccc6sc7ccccc7c6c5)cc34)cc2)cc1 ; CC1(C)c2ccccc2-c2ccc(N(c3ccccc3)c3cc4ccccc4c4c3oc3ccc(-c5ccc6sc7ccccc7c6c5)cc34)cc21]

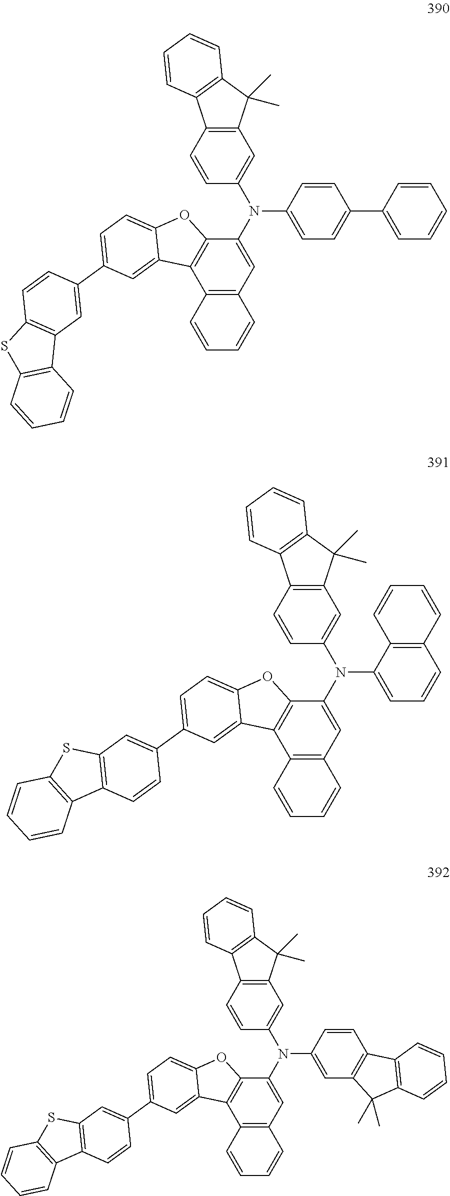 [SMILES: CC1(C)c2ccccc2-c2ccc(N(c3ccc(-c4ccccc4)cc3)c3cc4ccccc4c4c3oc3ccc(-c5ccc6sc7ccccc7c6c5)cc34)cc21 ; CC1(C)c2ccccc2-c2ccc(N(c3cccc4ccccc34)c3cc4ccccc4c4c3oc3ccc(-c5ccc6c(c5)sc5ccccc56)cc34)cc21 ; CC1(C)c2ccccc2-c2ccc(N(c3ccc4c(c3)C(C)(C)c3ccccc3-4)c3cc4ccccc4c4c3oc3ccc(-c5ccc6c(c5)sc5ccccc56)cc34)cc21]

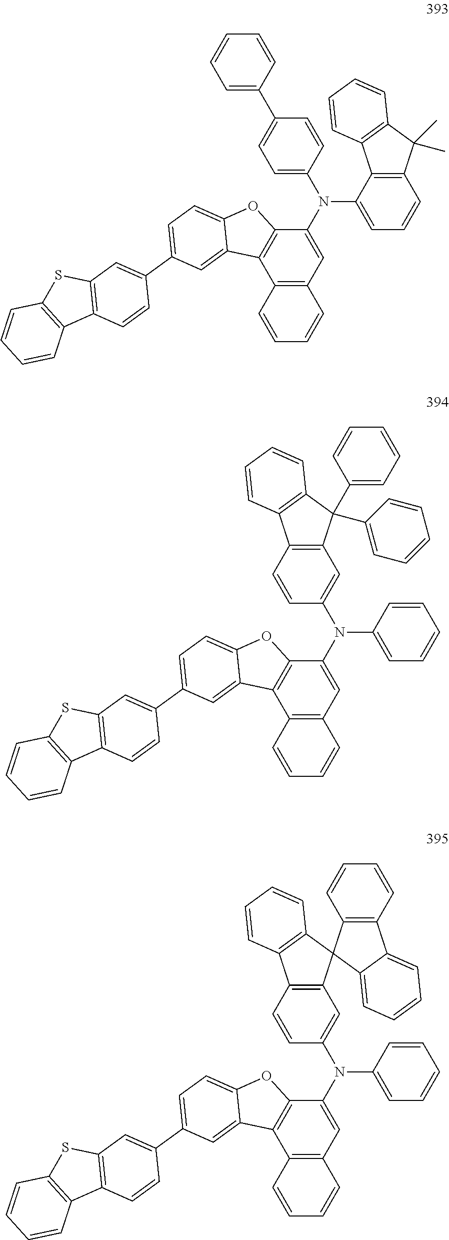 [SMILES: CC1(C)c2ccccc2-c2c(N(c3ccc(-c4ccccc4)cc3)c3cc4ccccc4c4c3oc3ccc(-c5ccc6c(c5)sc5ccccc56)cc34)cccc21 ; c1ccc(N(c2ccc3c(c2)C(c2ccccc2)(c2ccccc2)c2ccccc2-3)c2cc3ccccc3c3c2oc2ccc(-c4ccc5c(c4)sc4ccccc45)cc23)cc1 ; c1ccc(N(c2ccc3c(c2)C2(c4ccccc4-c4ccccc42)c2ccccc2-3)c2cc3ccccc3c3c2oc2ccc(-c4ccc5c(c4)sc4ccccc45)cc23)cc1]

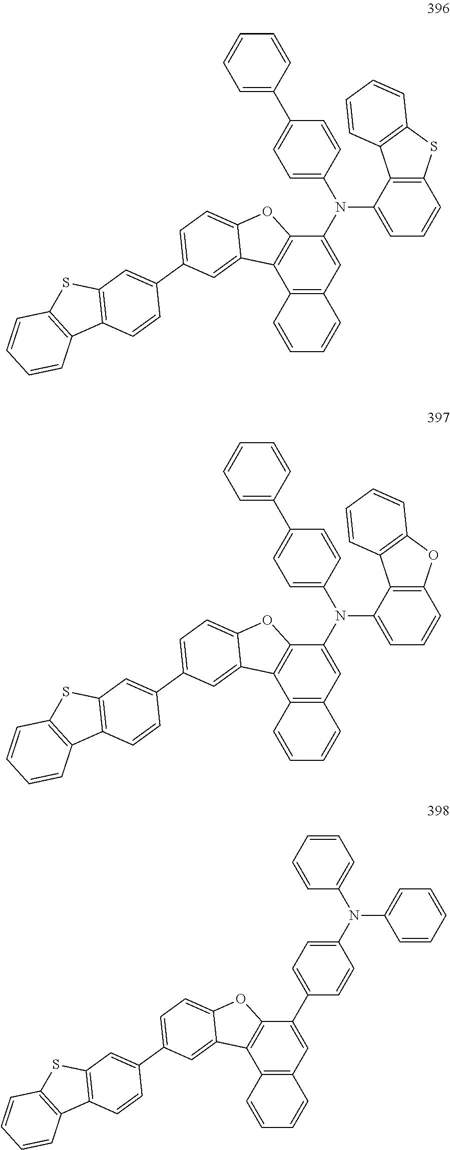 [SMILES: c1ccc(-c2ccc(N(c3cc4ccccc4c4c3oc3ccc(-c5ccc6c(c5)sc5ccccc56)cc34)c3cccc4sc5ccccc5c34)cc2)cc1 ; c1ccc(-c2ccc(N(c3cc4ccccc4c4c3oc3ccc(-c5ccc6c(c5)sc5ccccc56)cc34)c3cccc4oc5ccccc5c34)cc2)cc1 ; c1ccc(N(c2ccccc2)c2ccc(-c3cc4ccccc4c4c3oc3ccc(-c5ccc6c(c5)sc5ccccc56)cc34)cc2)cc1]

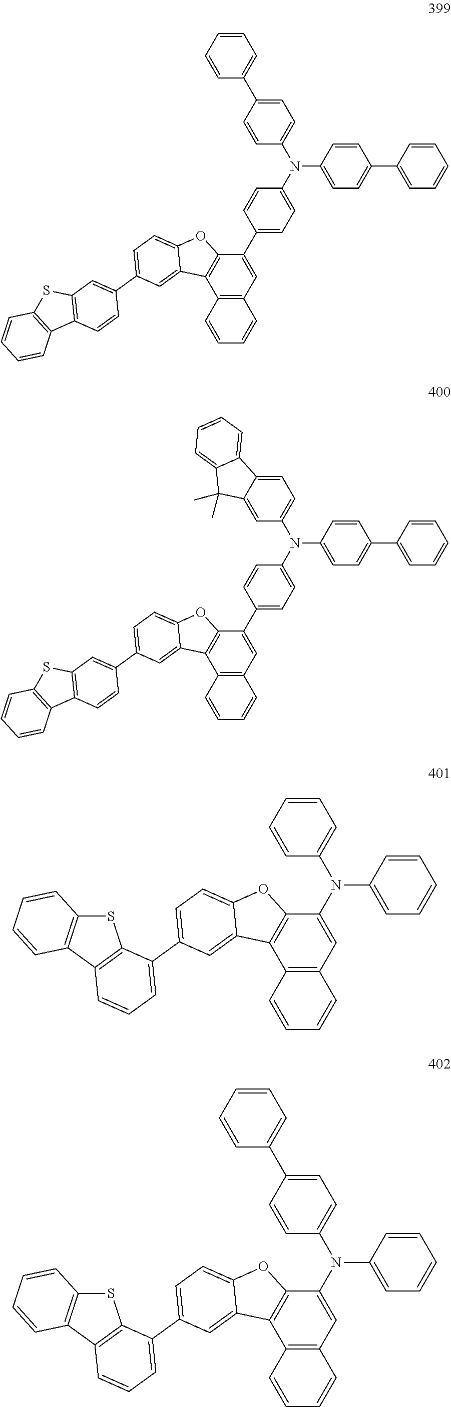 [SMILES: c1ccc(-c2ccc(N(c3ccc(-c4ccccc4)cc3)c3ccc(-c4cc5ccccc5c5c4oc4ccc(-c6ccc7c(c6)sc6ccccc67)cc45)cc3)cc2)cc1 ; CC1(C)c2ccccc2-c2ccc(N(c3ccc(-c4ccccc4)cc3)c3ccc(-c4cc5ccccc5c5c4oc4ccc(-c6ccc7c(c6)sc6ccccc67)cc45)cc3)cc21 ; c1ccc(N(c2ccccc2)c2cc3ccccc3c3c2oc2ccc(-c4cccc5c4sc4ccccc45)cc23)cc1 ; c1ccc(-c2ccc(N(c3ccccc3)c3cc4ccccc4c4c3oc3ccc(-c5cccc6c5sc5ccccc56)cc34)cc2)cc1]

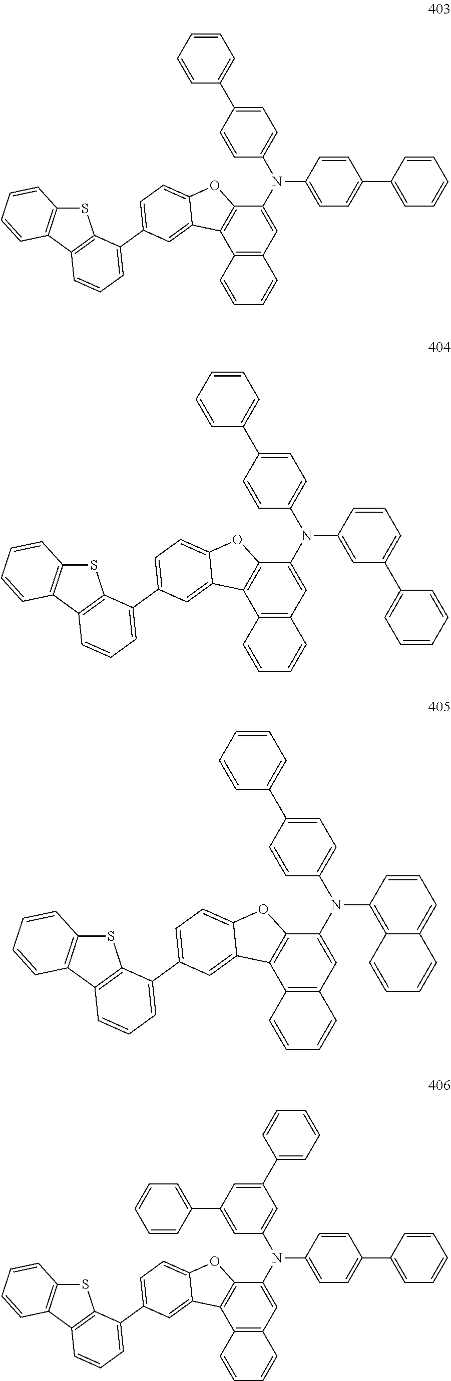 [SMILES: c1ccc(-c2ccc(N(c3ccc(-c4ccccc4)cc3)c3cc4ccccc4c4c3oc3ccc(-c5cccc6c5sc5ccccc56)cc34)cc2)cc1 ; c1ccc(-c2ccc(N(c3cccc(-c4ccccc4)c3)c3cc4ccccc4c4c3oc3ccc(-c5cccc6c5sc5ccccc56)cc34)cc2)cc1 ; c1ccc(-c2ccc(N(c3cccc4ccccc34)c3cc4ccccc4c4c3oc3ccc(-c5cccc6c5sc5ccccc56)cc34)cc2)cc1 ; c1ccc(-c2ccc(N(c3cc(-c4ccccc4)cc(-c4ccccc4)c3)c3cc4ccccc4c4c3oc3ccc(-c5cccc6c5sc5ccccc56)cc34)cc2)cc1]

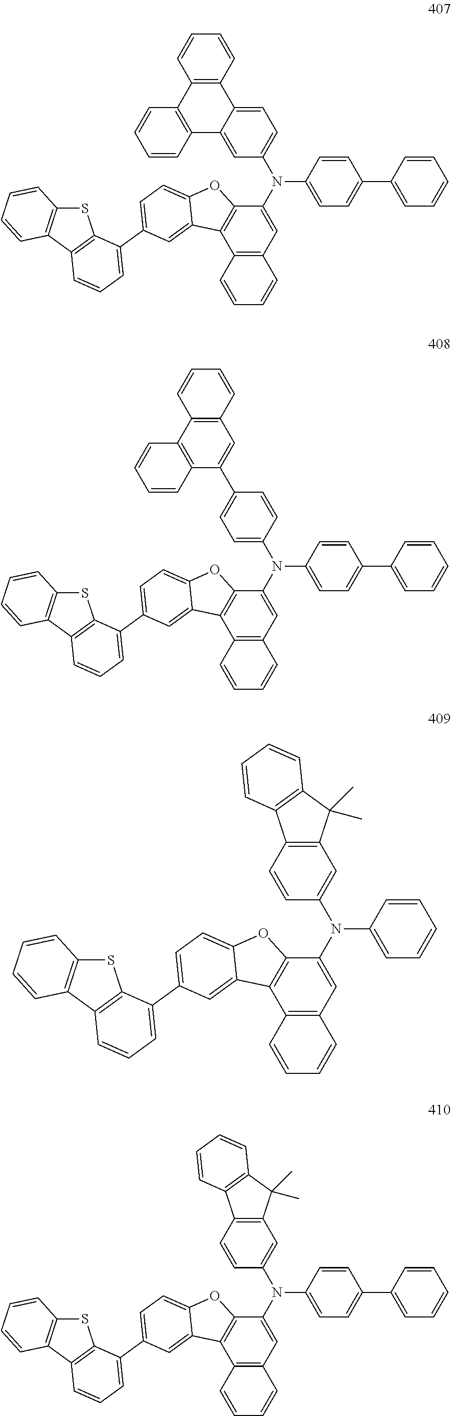 [SMILES: c1ccc(-c2ccc(N(c3ccc4c5ccccc5c5ccccc5c4c3)c3cc4ccccc4c4c3oc3ccc(-c5cccc6c5sc5ccccc56)cc34)cc2)cc1 ; c1ccc(-c2ccc(N(c3ccc(-c4cc5ccccc5c5ccccc45)cc3)c3cc4ccccc4c4c3oc3ccc(-c5cccc6c5sc5ccccc56)cc34)cc2)cc1 ; CC1(C)c2ccccc2-c2ccc(N(c3ccccc3)c3cc4ccccc4c4c3oc3ccc(-c5cccc6c5sc5ccccc56)cc34)cc21 ; CC1(C)c2ccccc2-c2ccc(N(c3ccc(-c4ccccc4)cc3)c3cc4ccccc4c4c3oc3ccc(-c5cccc6c5sc5ccccc56)cc34)cc21]

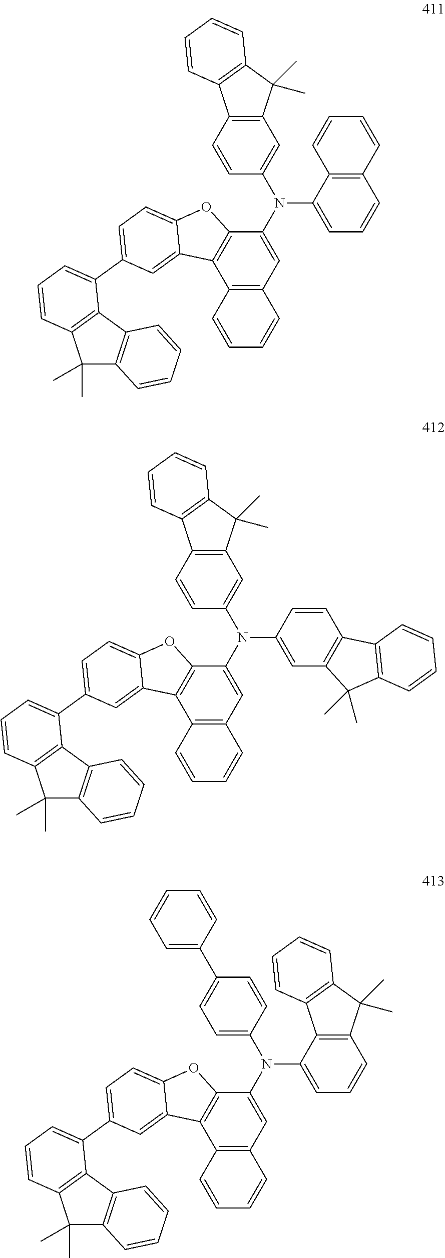 [SMILES: CC1(C)c2ccccc2-c2ccc(N(c3cccc4ccccc34)c3cc4ccccc4c4c3oc3ccc(-c5cccc6c5-c5ccccc5C6(C)C)cc34)cc21 ; CC1(C)c2ccccc2-c2ccc(N(c3ccc4c(c3)C(C)(C)c3ccccc3-4)c3cc4ccccc4c4c3oc3ccc(-c5cccc6c5-c5ccccc5C6(C)C)cc34)cc21 ; CC1(C)c2ccccc2-c2c(-c3ccc4oc5c(N(c6ccc(-c7ccccc7)cc6)c6cccc7c6-c6ccccc6C7(C)C)cc6ccccc6c5c4c3)cccc21]

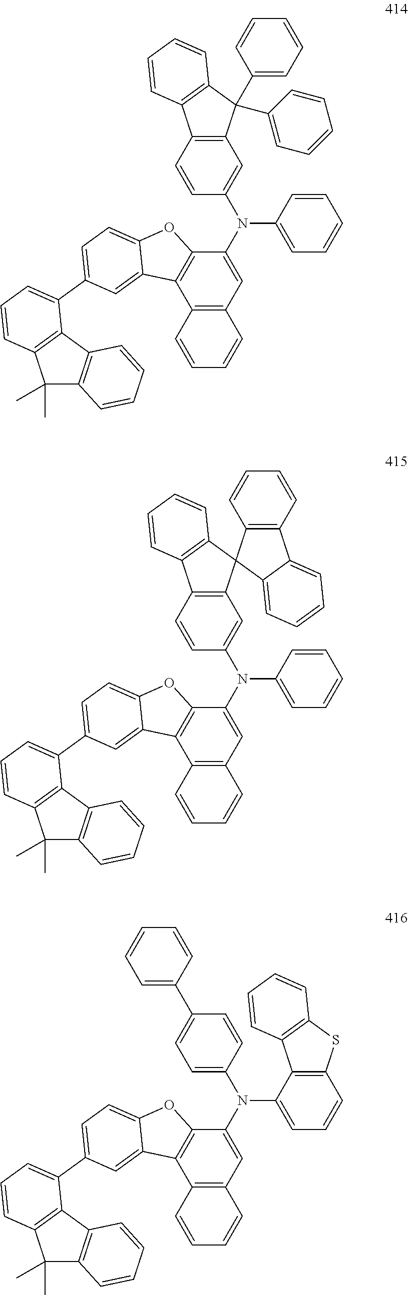 [SMILES: CC1(C)c2ccccc2-c2c(-c3ccc4oc5c(N(c6ccccc6)c6ccc7c(c6)C(c6ccccc6)(c6ccccc6)c6ccccc6-7)cc6ccccc6c5c4c3)cccc21 ; CC1(C)c2ccccc2-c2c(-c3ccc4oc5c(N(c6ccccc6)c6ccc7c(c6)C6(c8ccccc8-c8ccccc86)c6ccccc6-7)cc6ccccc6c5c4c3)cccc21 ; CC1(C)c2ccccc2-c2c(-c3ccc4oc5c(N(c6ccc(-c7ccccc7)cc6)c6cccc7sc8ccccc8c67)cc6ccccc6c5c4c3)cccc21]

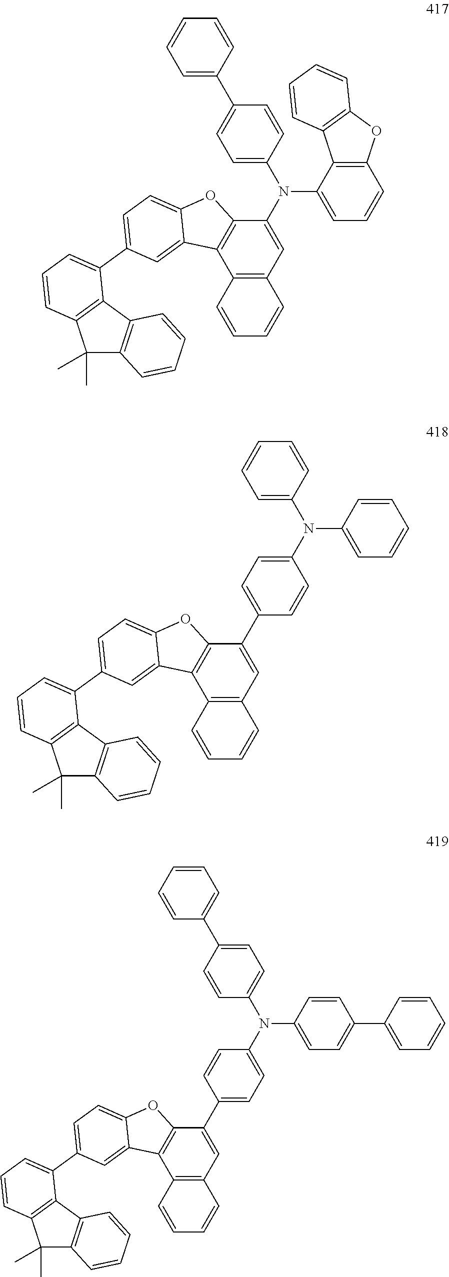 [SMILES: CC1(C)c2ccccc2-c2c(-c3ccc4oc5c(N(c6ccc(-c7ccccc7)cc6)c6cccc7oc8ccccc8c67)cc6ccccc6c5c4c3)cccc21 ; CC1(C)c2ccccc2-c2c(-c3ccc4oc5c(-c6ccc(N(c7ccccc7)c7ccccc7)cc6)cc6ccccc6c5c4c3)cccc21 ; CC1(C)c2ccccc2-c2c(-c3ccc4oc5c(-c6ccc(N(c7ccc(-c8ccccc8)cc7)c7ccc(-c8ccccc8)cc7)cc6)cc6ccccc6c5c4c3)cccc21]

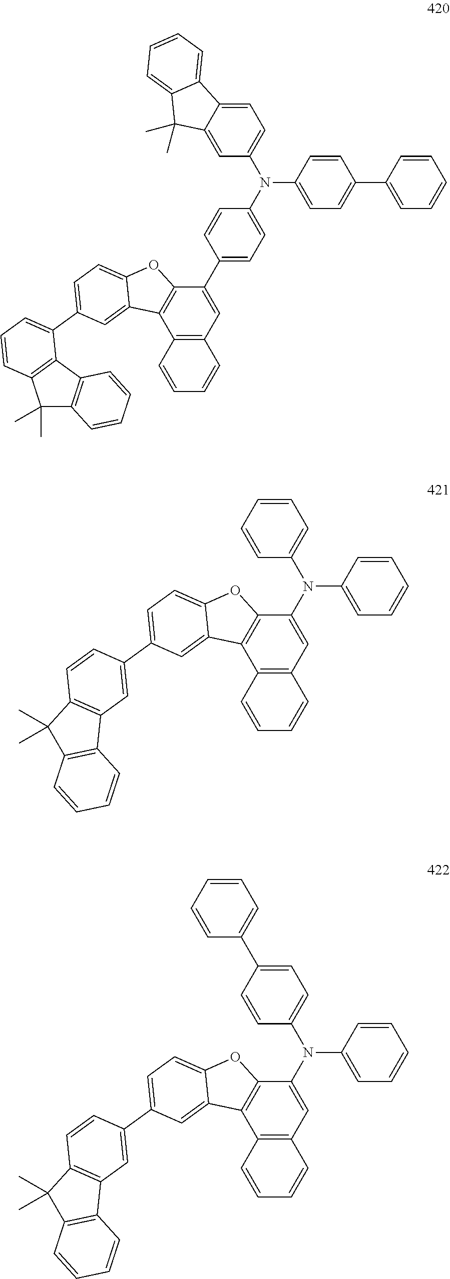 [SMILES: CC1(C)c2ccccc2-c2ccc(N(c3ccc(-c4ccccc4)cc3)c3ccc(-c4cc5ccccc5c5c4oc4ccc(-c6cccc7c6-c6ccccc6C7(C)C)cc45)cc3)cc21 ; CC1(C)c2ccccc2-c2cc(-c3ccc4oc5c(N(c6ccccc6)c6ccccc6)cc6ccccc6c5c4c3)ccc21 ; CC1(C)c2ccccc2-c2cc(-c3ccc4oc5c(N(c6ccccc6)c6ccc(-c7ccccc7)cc6)cc6ccccc6c5c4c3)ccc21]

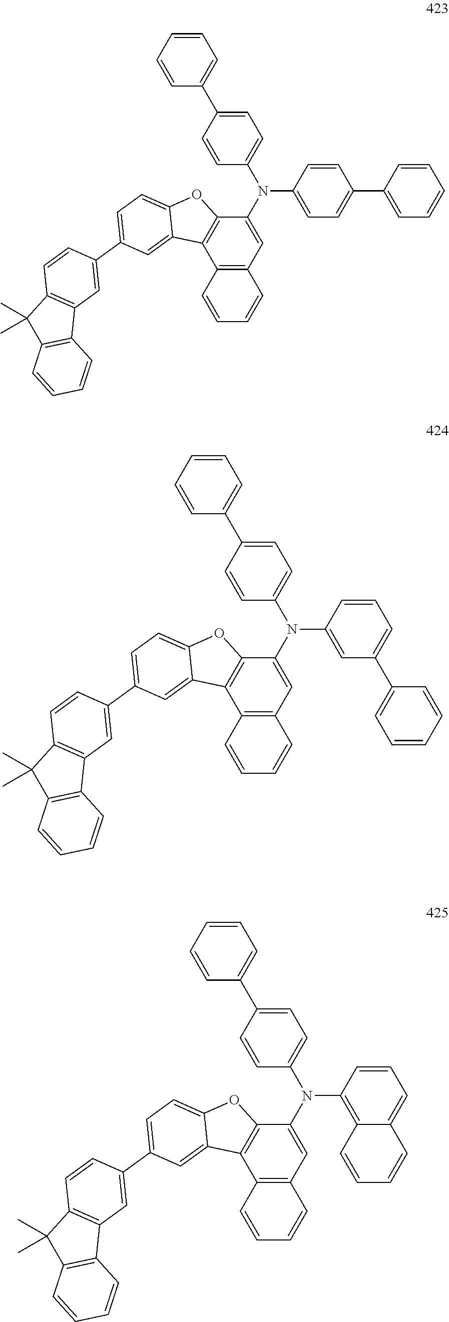 [SMILES: CC1(C)c2ccccc2-c2cc(-c3ccc4oc5c(N(c6ccc(-c7ccccc7)cc6)c6ccc(-c7ccccc7)cc6)cc6ccccc6c5c4c3)ccc21 ; CC1(C)c2ccccc2-c2cc(-c3ccc4oc5c(N(c6ccc(-c7ccccc7)cc6)c6cccc(-c7ccccc7)c6)cc6ccccc6c5c4c3)ccc21 ; CC1(C)c2ccccc2-c2cc(-c3ccc4oc5c(N(c6ccc(-c7ccccc7)cc6)c6cccc7ccccc67)cc6ccccc6c5c4c3)ccc21]

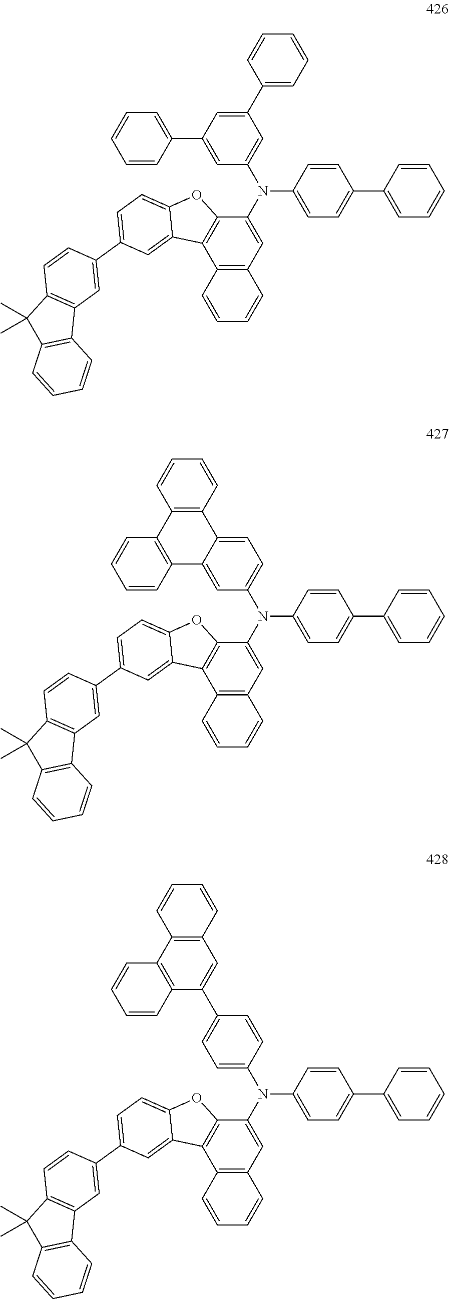 [SMILES: CC1(C)c2ccccc2-c2cc(-c3ccc4oc5c(N(c6ccc(-c7ccccc7)cc6)c6cc(-c7ccccc7)cc(-c7ccccc7)c6)cc6ccccc6c5c4c3)ccc21 ; CC1(C)c2ccccc2-c2cc(-c3ccc4oc5c(N(c6ccc(-c7ccccc7)cc6)c6ccc7c8ccccc8c8ccccc8c7c6)cc6ccccc6c5c4c3)ccc21 ; CC1(C)c2ccccc2-c2cc(-c3ccc4oc5c(N(c6ccc(-c7ccccc7)cc6)c6ccc(-c7cc8ccccc8c8ccccc78)cc6)cc6ccccc6c5c4c3)ccc21]

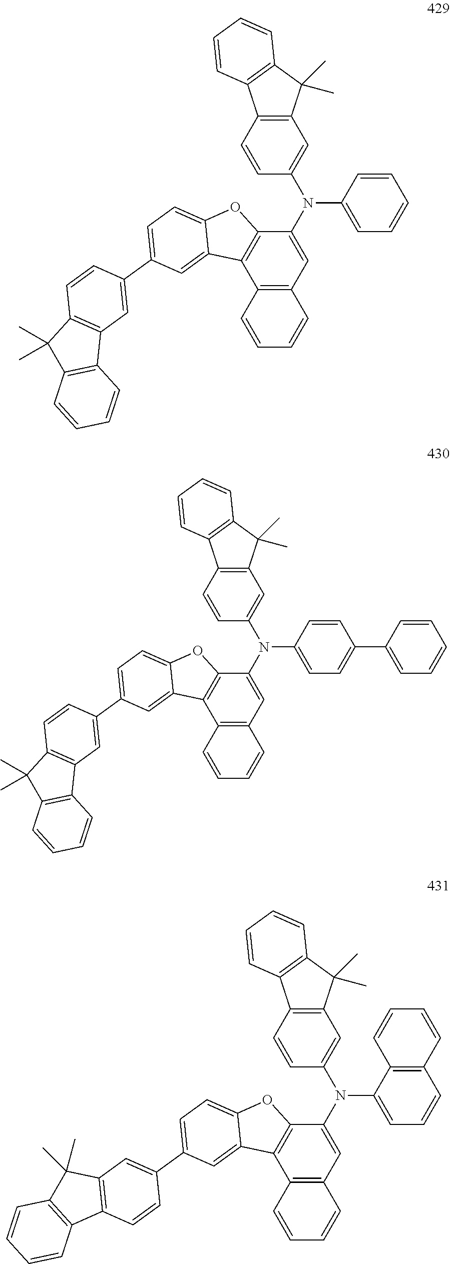 [SMILES: CC1(C)c2ccccc2-c2cc(-c3ccc4oc5c(N(c6ccccc6)c6ccc7c(c6)C(C)(C)c6ccccc6-7)cc6ccccc6c5c4c3)ccc21 ; CC1(C)c2ccccc2-c2cc(-c3ccc4oc5c(N(c6ccc(-c7ccccc7)cc6)c6ccc7c(c6)C(C)(C)c6ccccc6-7)cc6ccccc6c5c4c3)ccc21 ; CC1(C)c2ccccc2-c2ccc(-c3ccc4oc5c(N(c6ccc7c(c6)C(C)(C)c6ccccc6-7)c6cccc7ccccc67)cc6ccccc6c5c4c3)cc21]

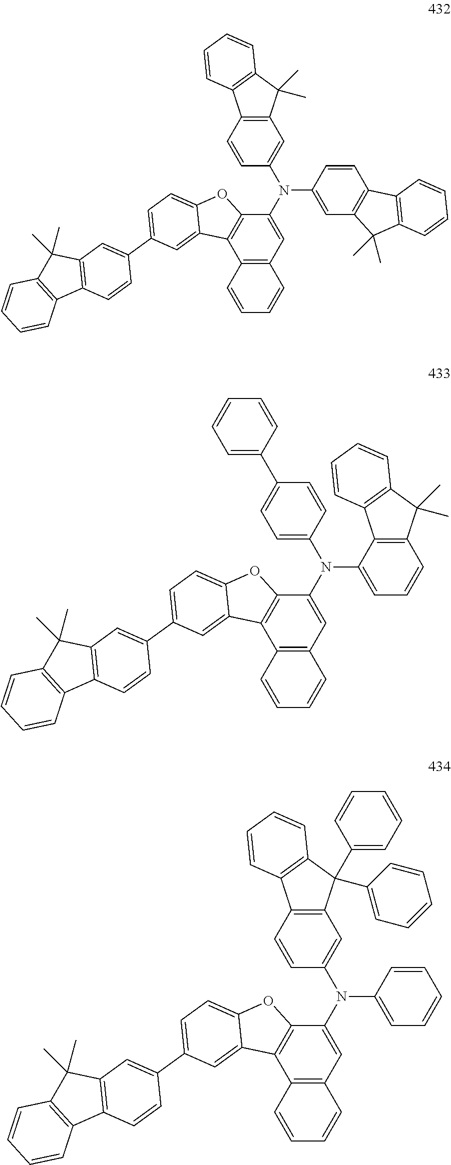 [SMILES: CC1(C)c2ccccc2-c2ccc(-c3ccc4oc5c(N(c6ccc7c(c6)C(C)(C)c6ccccc6-7)c6ccc7c(c6)C(C)(C)c6ccccc6-7)cc6ccccc6c5c4c3)cc21 ; CC1(C)c2ccccc2-c2ccc(-c3ccc4oc5c(N(c6ccc(-c7ccccc7)cc6)c6cccc7c6-c6ccccc6C7(C)C)cc6ccccc6c5c4c3)cc21 ; CC1(C)c2ccccc2-c2ccc(-c3ccc4oc5c(N(c6ccccc6)c6ccc7c(c6)C(c6ccccc6)(c6ccccc6)c6ccccc6-7)cc6ccccc6c5c4c3)cc21]

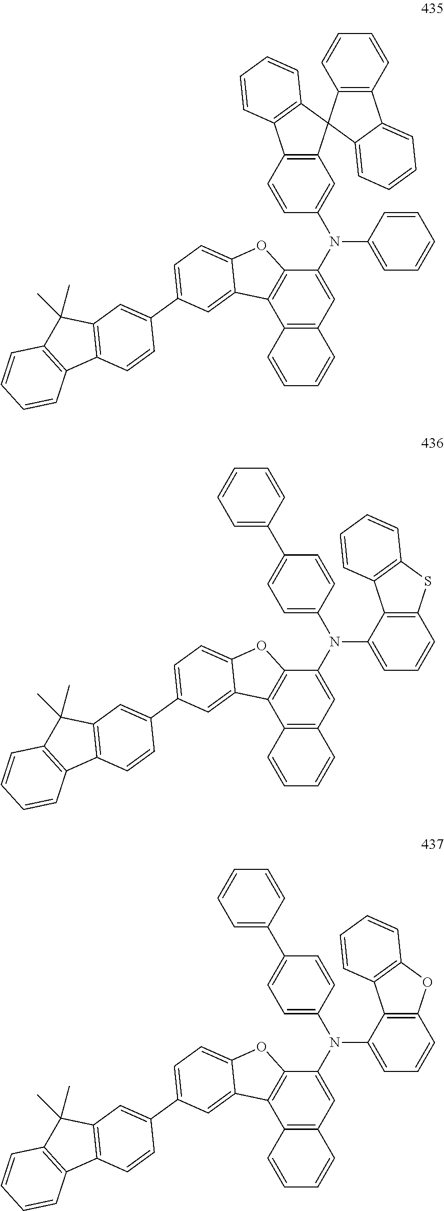 [SMILES: CC1(C)c2ccccc2-c2ccc(-c3ccc4oc5c(N(c6ccccc6)c6ccc7c(c6)C6(c8ccccc8-c8ccccc86)c6ccccc6-7)cc6ccccc6c5c4c3)cc21 ; CC1(C)c2ccccc2-c2ccc(-c3ccc4oc5c(N(c6ccc(-c7ccccc7)cc6)c6cccc7sc8ccccc8c67)cc6ccccc6c5c4c3)cc21 ; CC1(C)c2ccccc2-c2ccc(-c3ccc4oc5c(N(c6ccc(-c7ccccc7)cc6)c6cccc7oc8ccccc8c67)cc6ccccc6c5c4c3)cc21]

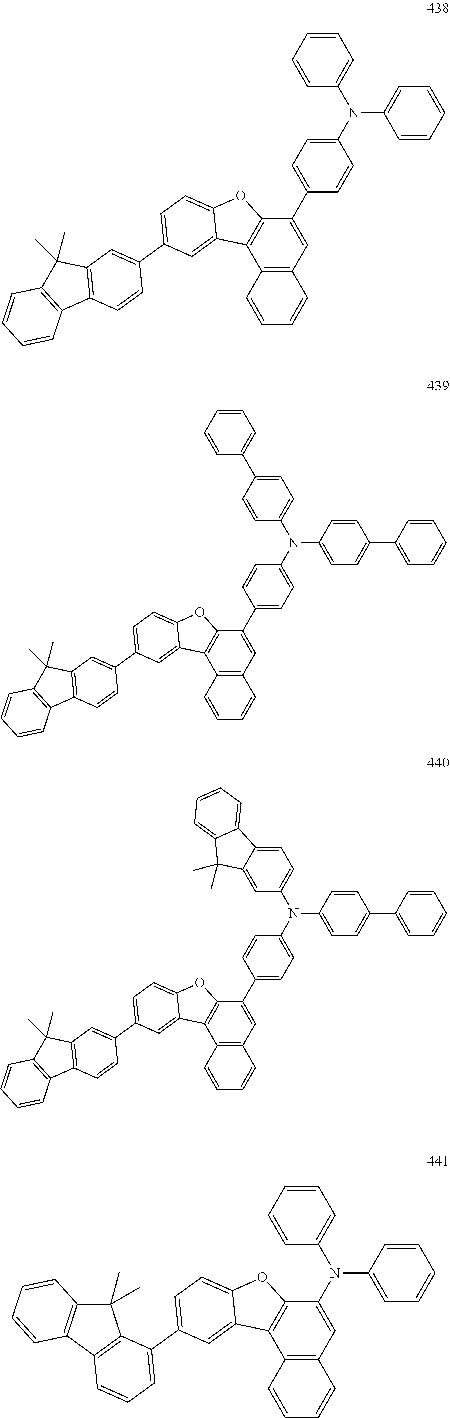 [SMILES: CC1(C)c2ccccc2-c2ccc(-c3ccc4oc5c(-c6ccc(N(c7ccccc7)c7ccccc7)cc6)cc6ccccc6c5c4c3)cc21 ; CC1(C)c2ccccc2-c2ccc(-c3ccc4oc5c(-c6ccc(N(c7ccc(-c8ccccc8)cc7)c7ccc(-c8ccccc8)cc7)cc6)cc6ccccc6c5c4c3)cc21 ; CC1(C)c2ccccc2-c2ccc(-c3ccc4oc5c(-c6ccc(N(c7ccc(-c8ccccc8)cc7)c7ccc8c(c7)C(C)(C)c7ccccc7-8)cc6)cc6ccccc6c5c4c3)cc21 ; CC1(C)c2ccccc2-c2cccc(-c3ccc4oc5c(N(c6ccccc6)c6ccccc6)cc6ccccc6c5c4c3)c21]

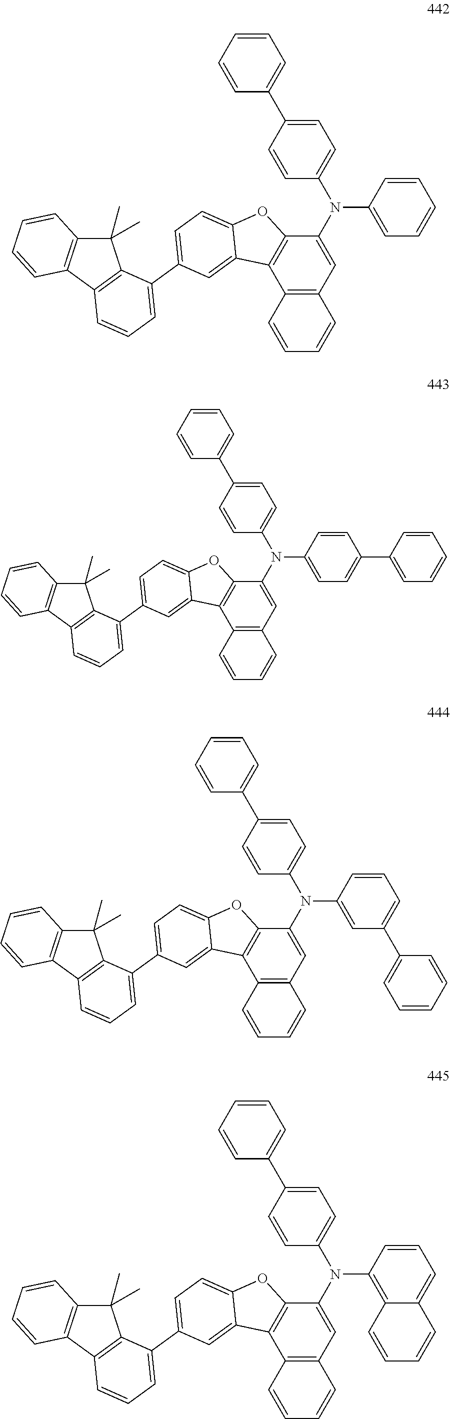 [SMILES: CC1(C)c2ccccc2-c2cccc(-c3ccc4oc5c(N(c6ccccc6)c6ccc(-c7ccccc7)cc6)cc6ccccc6c5c4c3)c21 ; CC1(C)c2ccccc2-c2cccc(-c3ccc4oc5c(N(c6ccc(-c7ccccc7)cc6)c6ccc(-c7ccccc7)cc6)cc6ccccc6c5c4c3)c21 ; CC1(C)c2ccccc2-c2cccc(-c3ccc4oc5c(N(c6ccc(-c7ccccc7)cc6)c6cccc(-c7ccccc7)c6)cc6ccccc6c5c4c3)c21 ; CC1(C)c2ccccc2-c2cccc(-c3ccc4oc5c(N(c6ccc(-c7ccccc7)cc6)c6cccc7ccccc67)cc6ccccc6c5c4c3)c21]

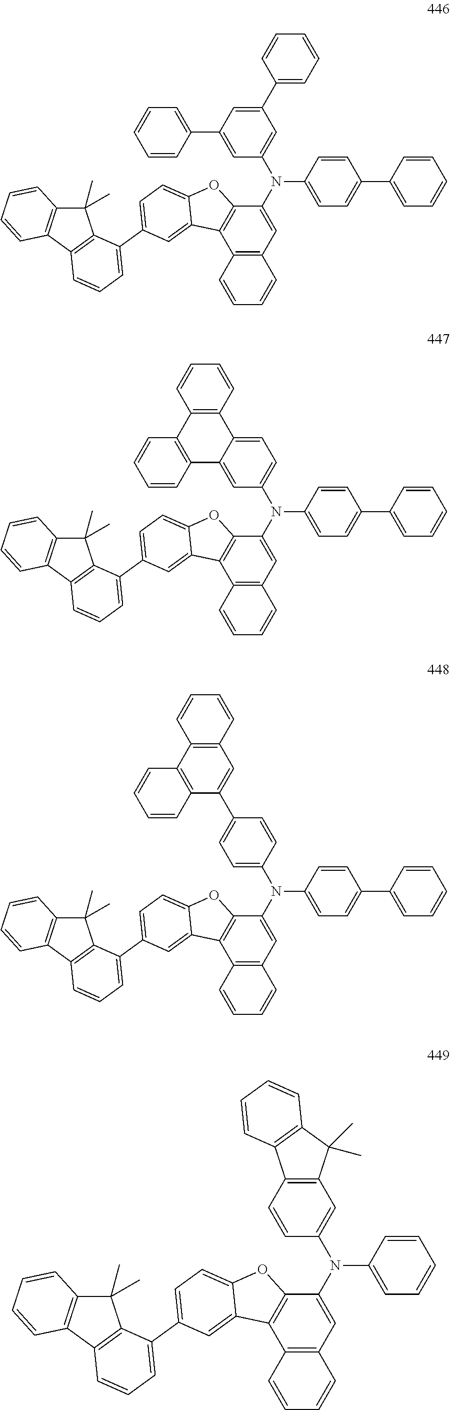 [SMILES: CC1(C)c2ccccc2-c2cccc(-c3ccc4oc5c(N(c6ccc(-c7ccccc7)cc6)c6cc(-c7ccccc7)cc(-c7ccccc7)c6)cc6ccccc6c5c4c3)c21 ; CC1(C)c2ccccc2-c2cccc(-c3ccc4oc5c(N(c6ccc(-c7ccccc7)cc6)c6ccc7c8ccccc8c8ccccc8c7c6)cc6ccccc6c5c4c3)c21 ; CC1(C)c2ccccc2-c2cccc(-c3ccc4oc5c(N(c6ccc(-c7ccccc7)cc6)c6ccc(-c7cc8ccccc8c8ccccc78)cc6)cc6ccccc6c5c4c3)c21 ; CC1(C)c2ccccc2-c2ccc(N(c3ccccc3)c3cc4ccccc4c4c3oc3ccc(-c5cccc6c5C(C)(C)c5ccccc5-6)cc34)cc21]

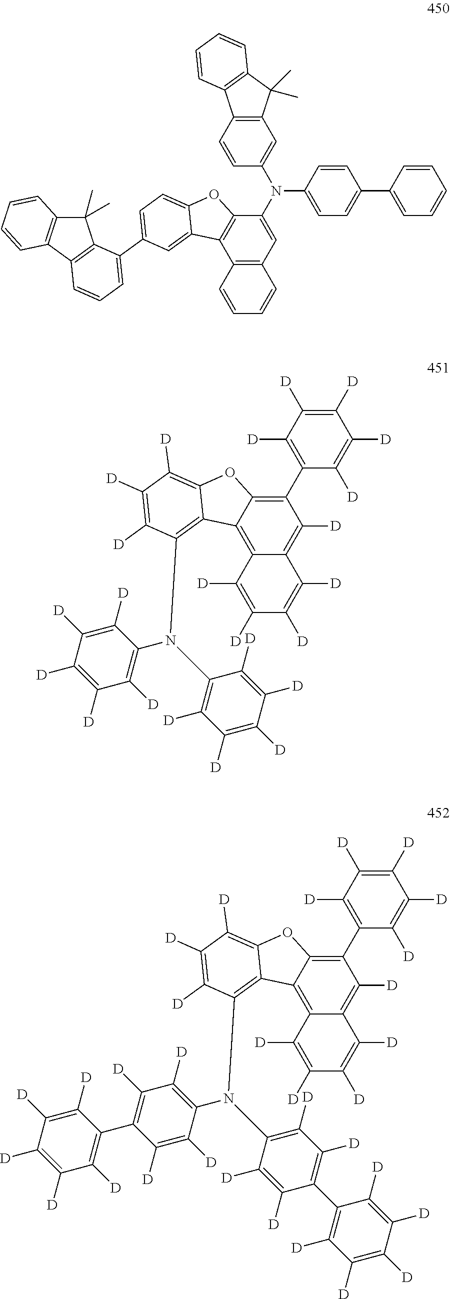

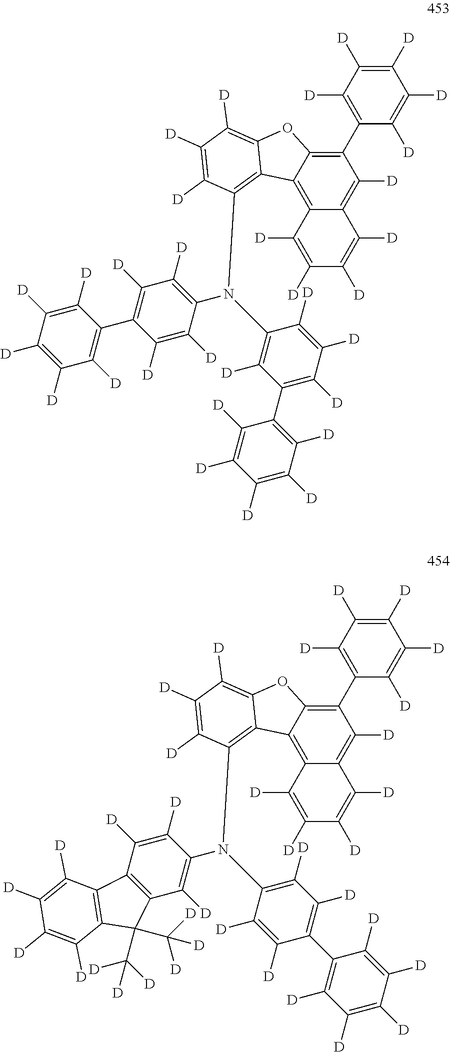

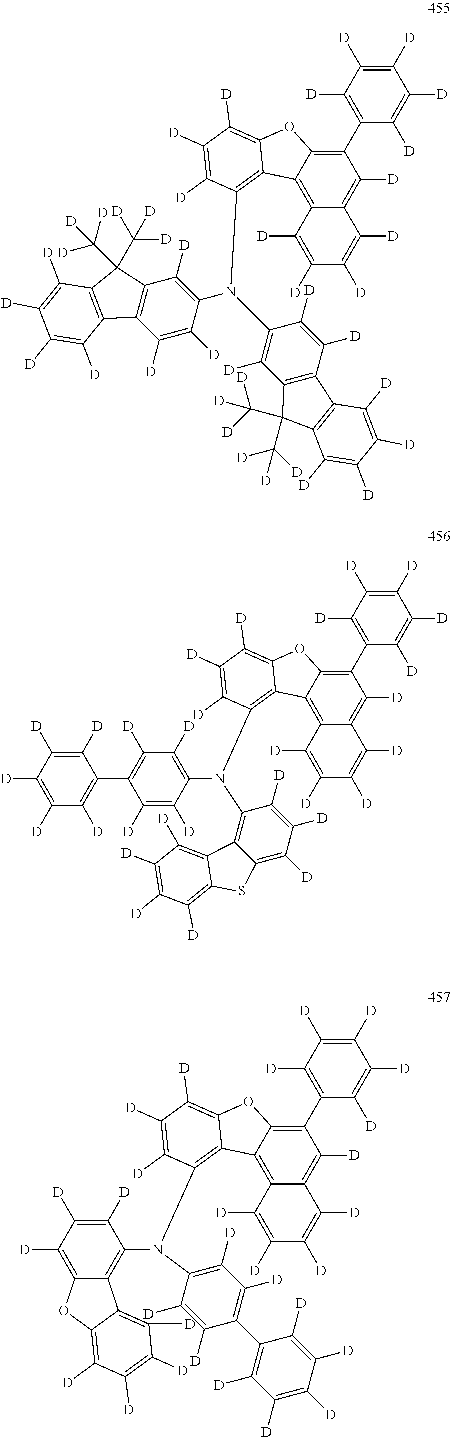

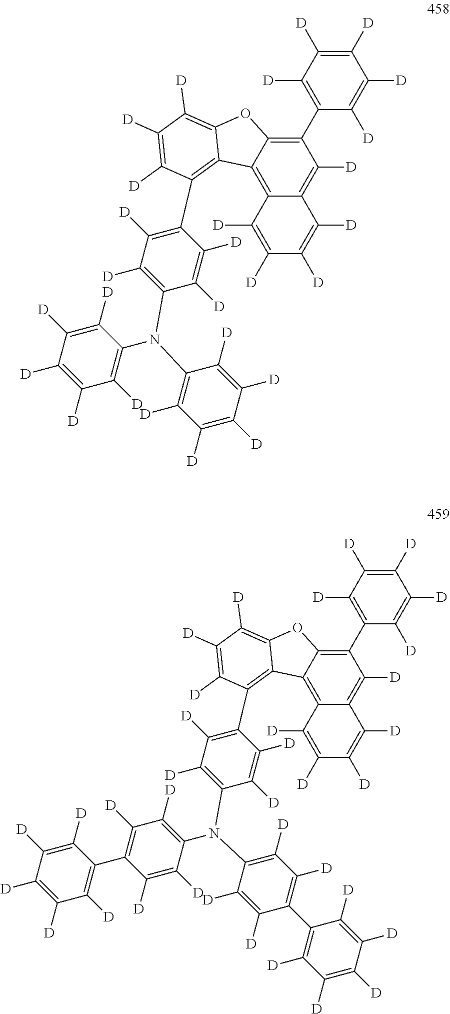

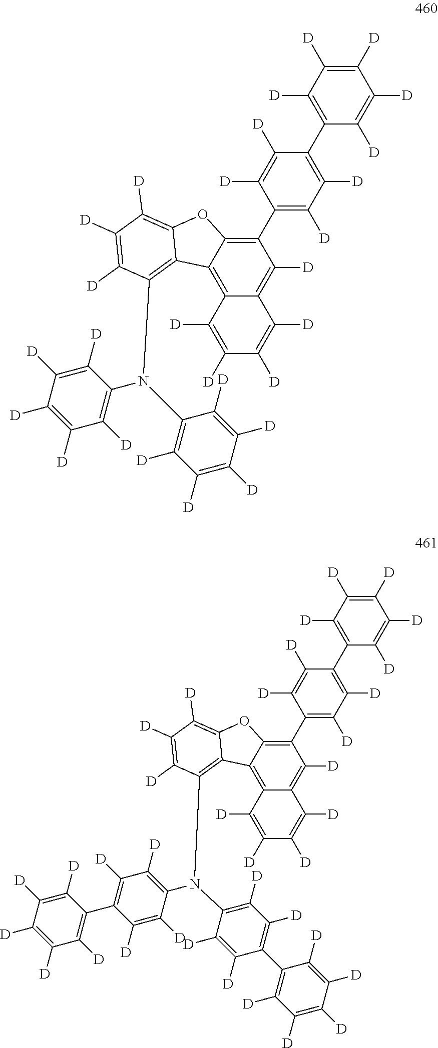

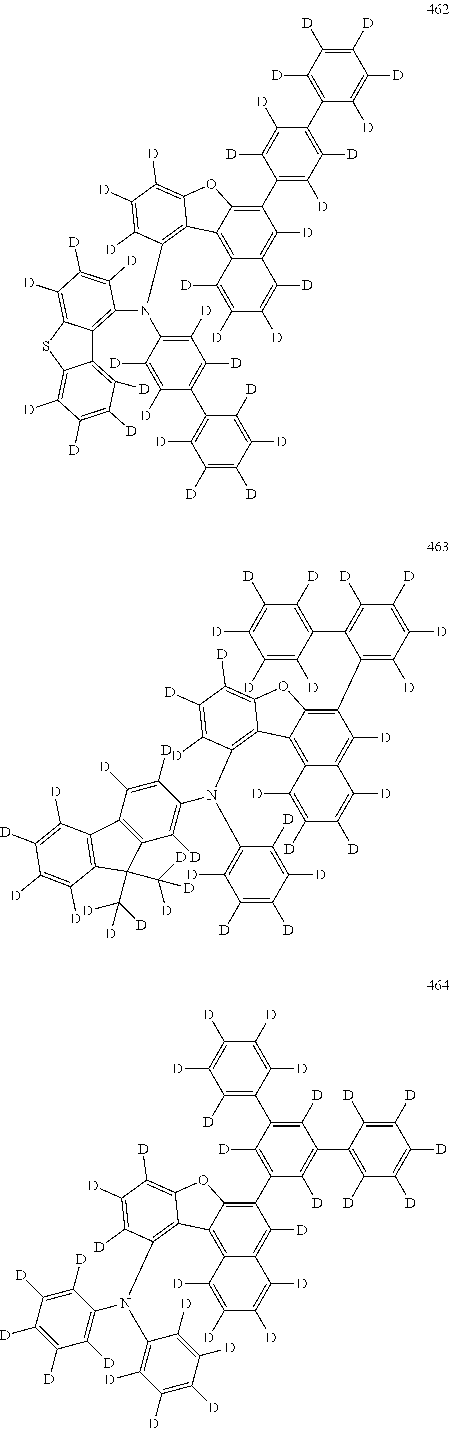

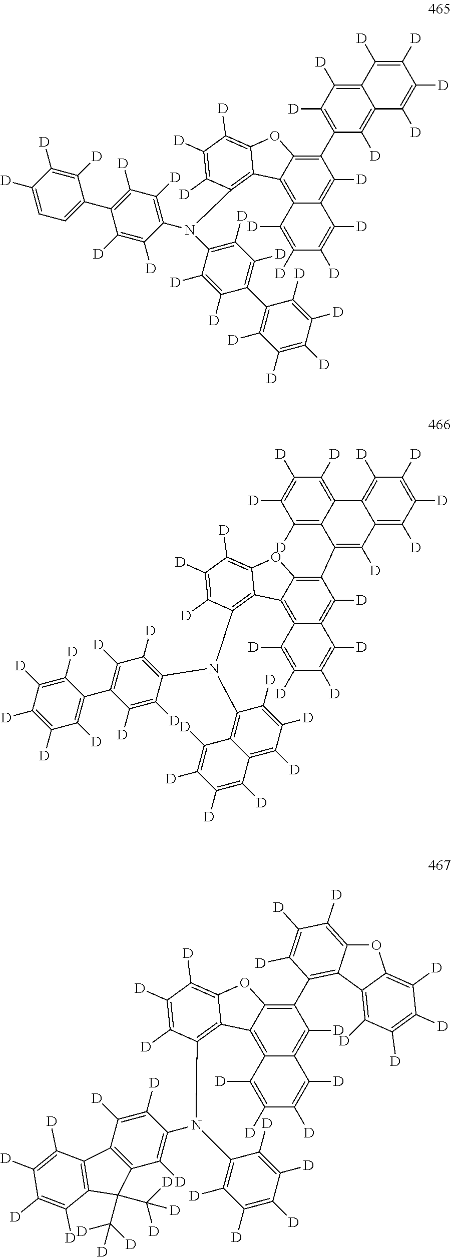

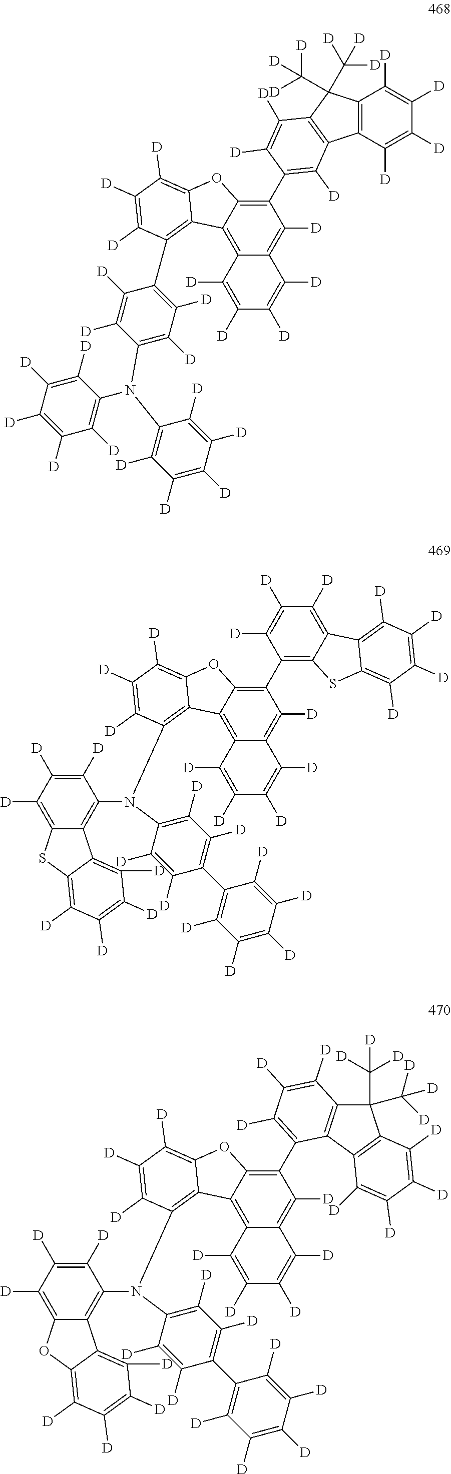

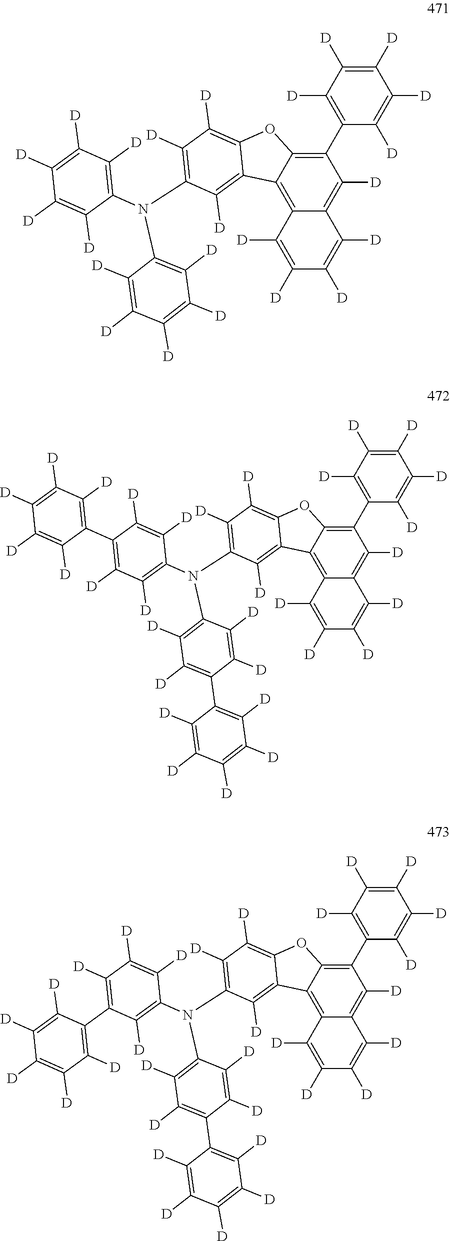

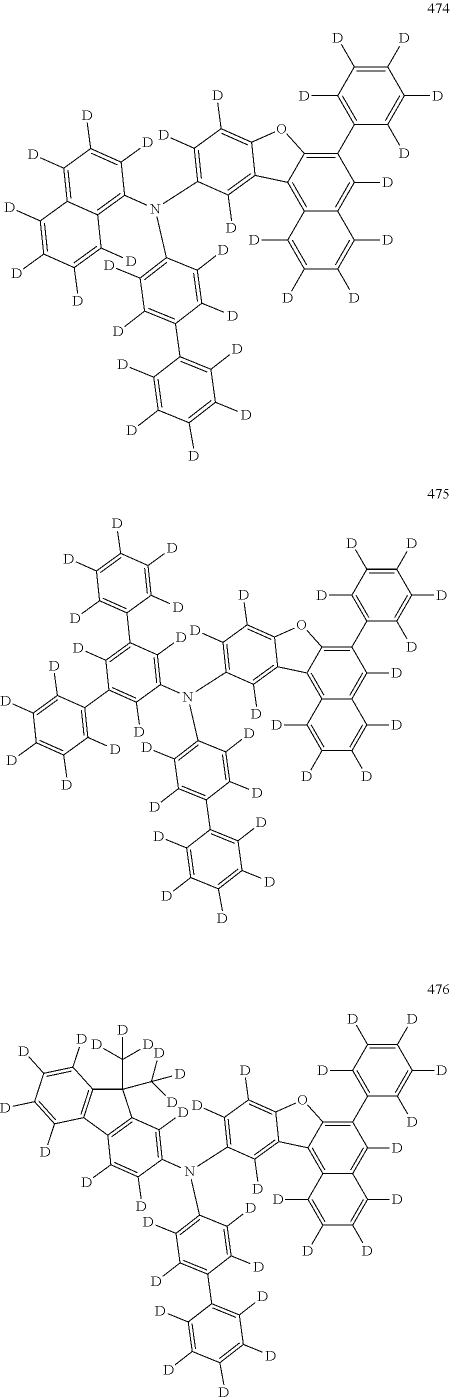

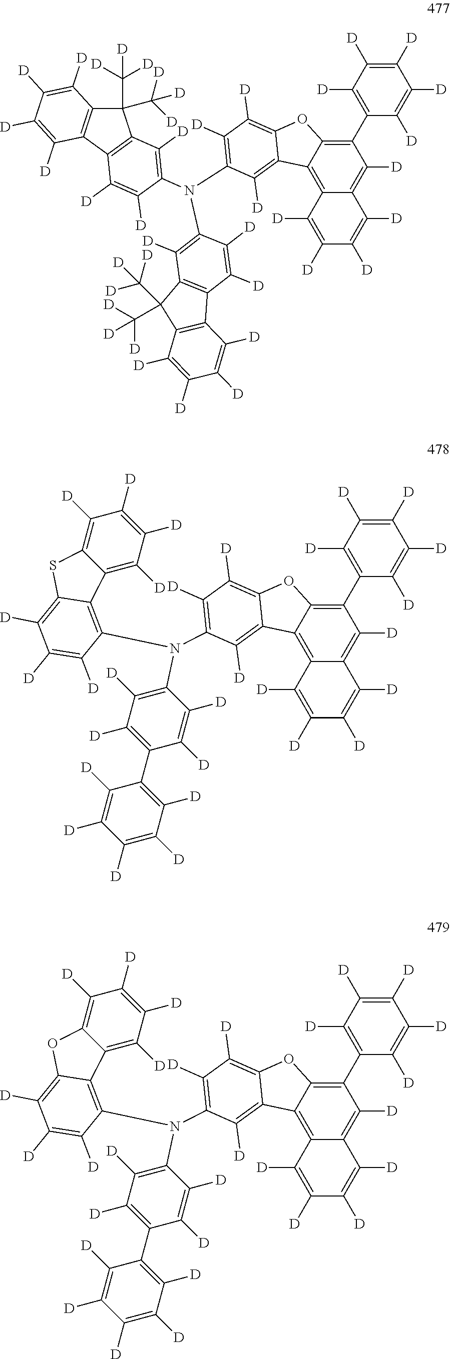

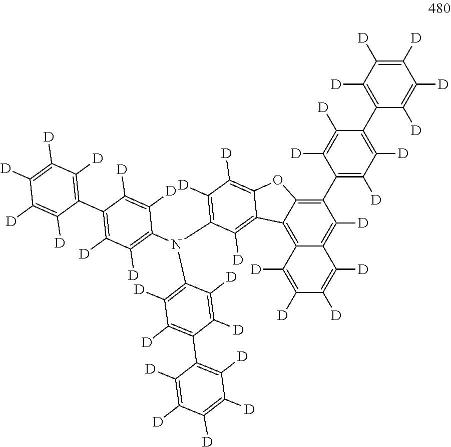 [SMILES: [2H]c1c([2H])c([2H])c(-c2c([2H])c([2H])c(-c3c([2H])c4c([2H])c([2H])c([2H])c([2H])c4c4c3oc3c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c34)c([2H])c2[2H])c([2H])c1[2H]]

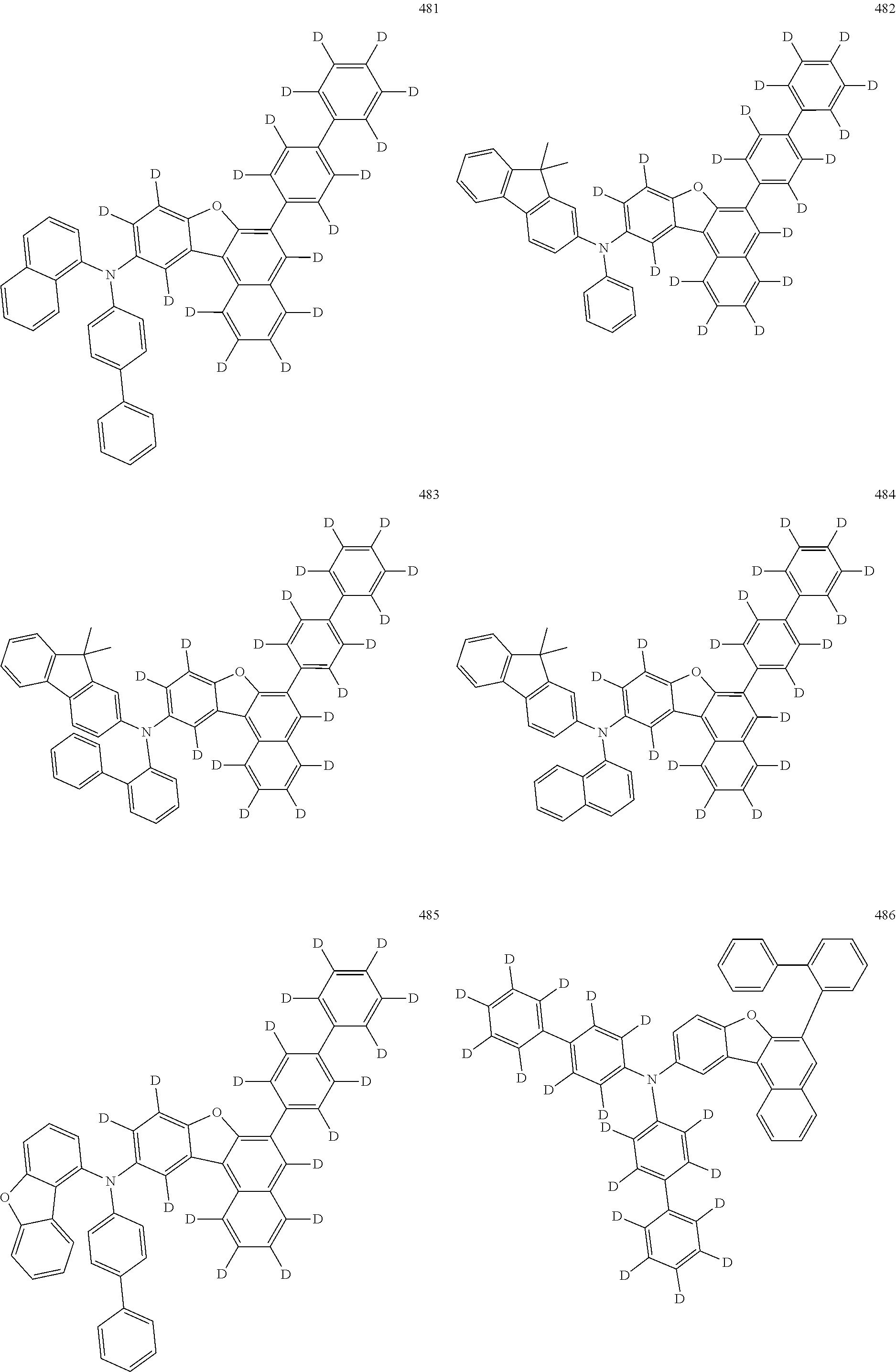

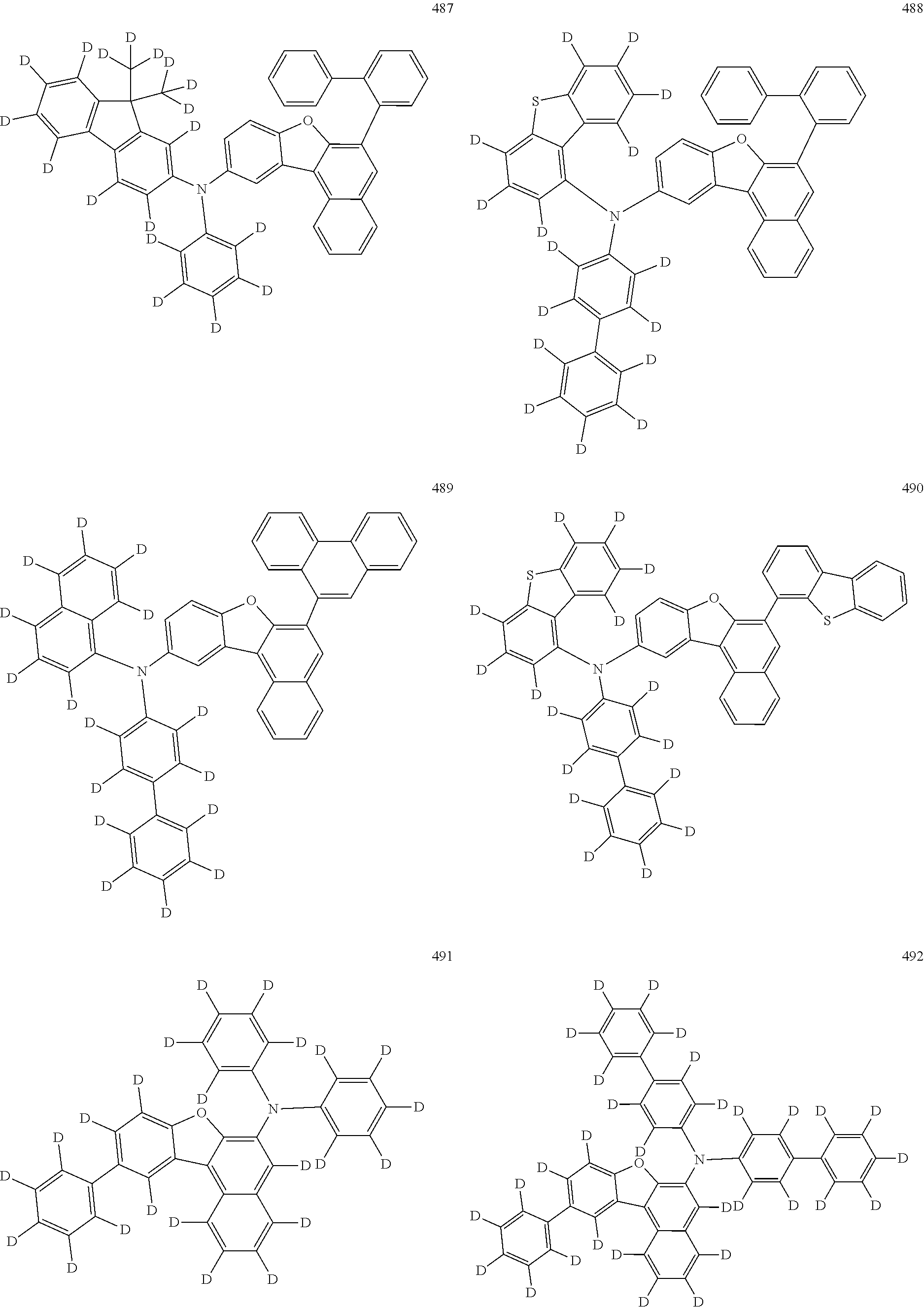

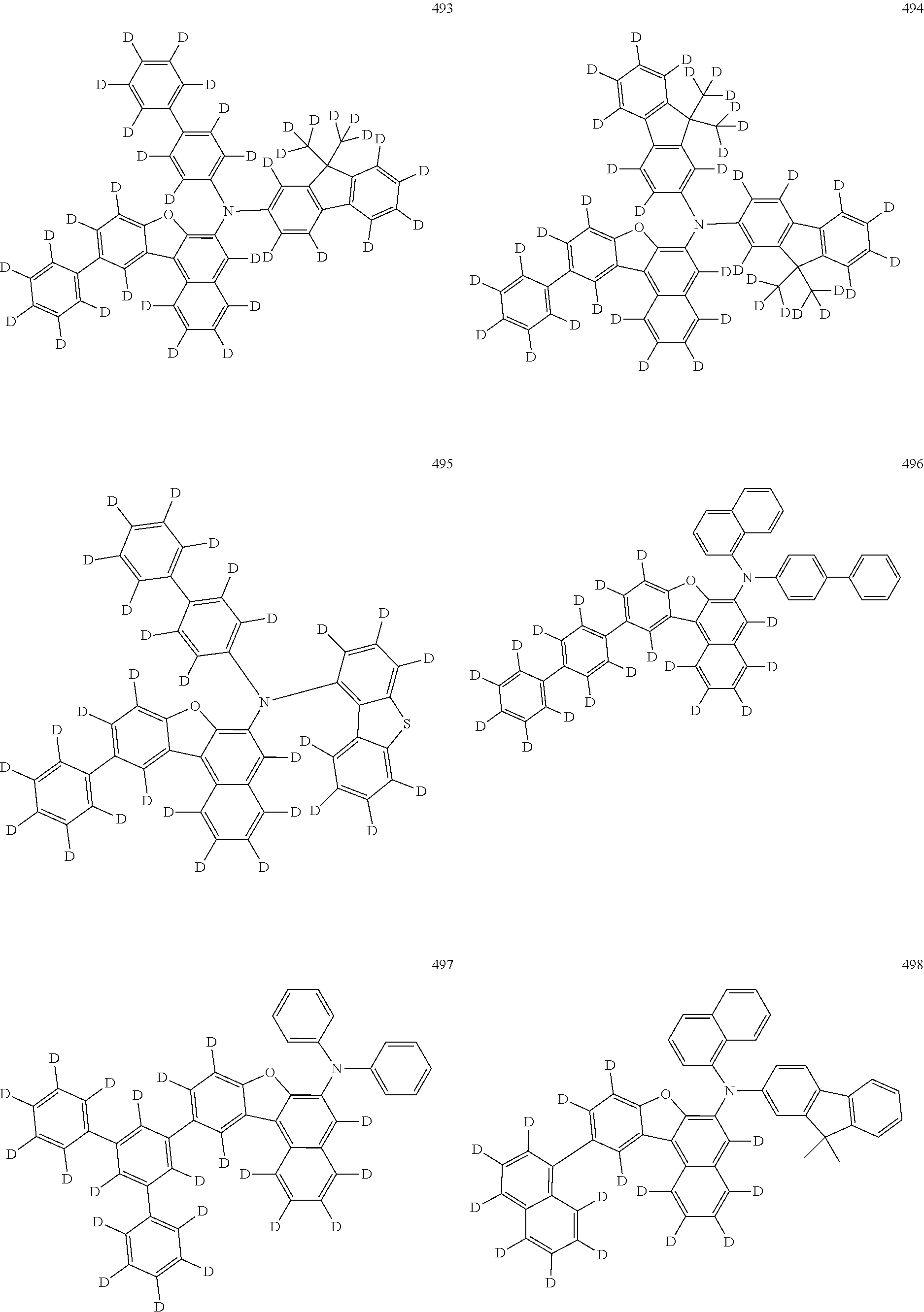

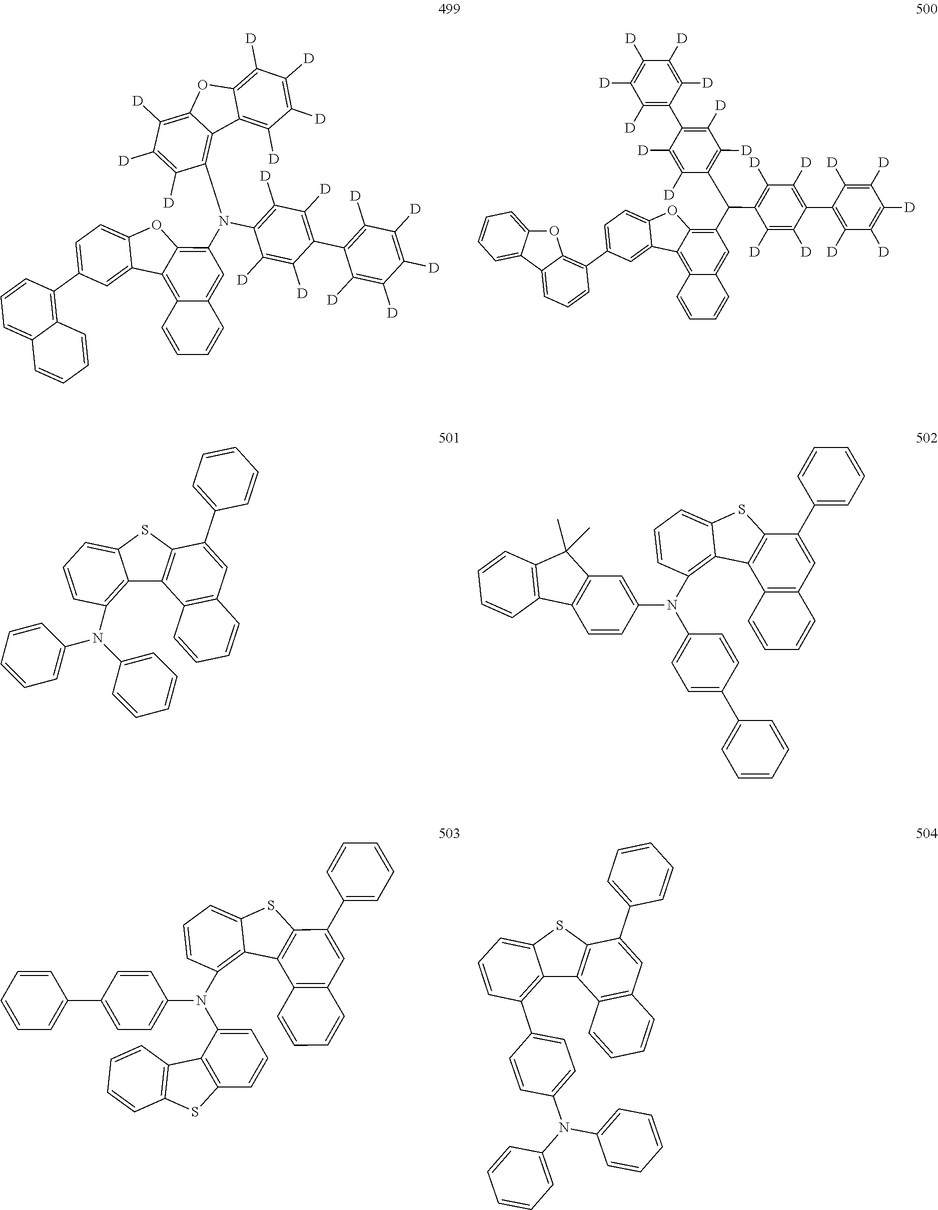

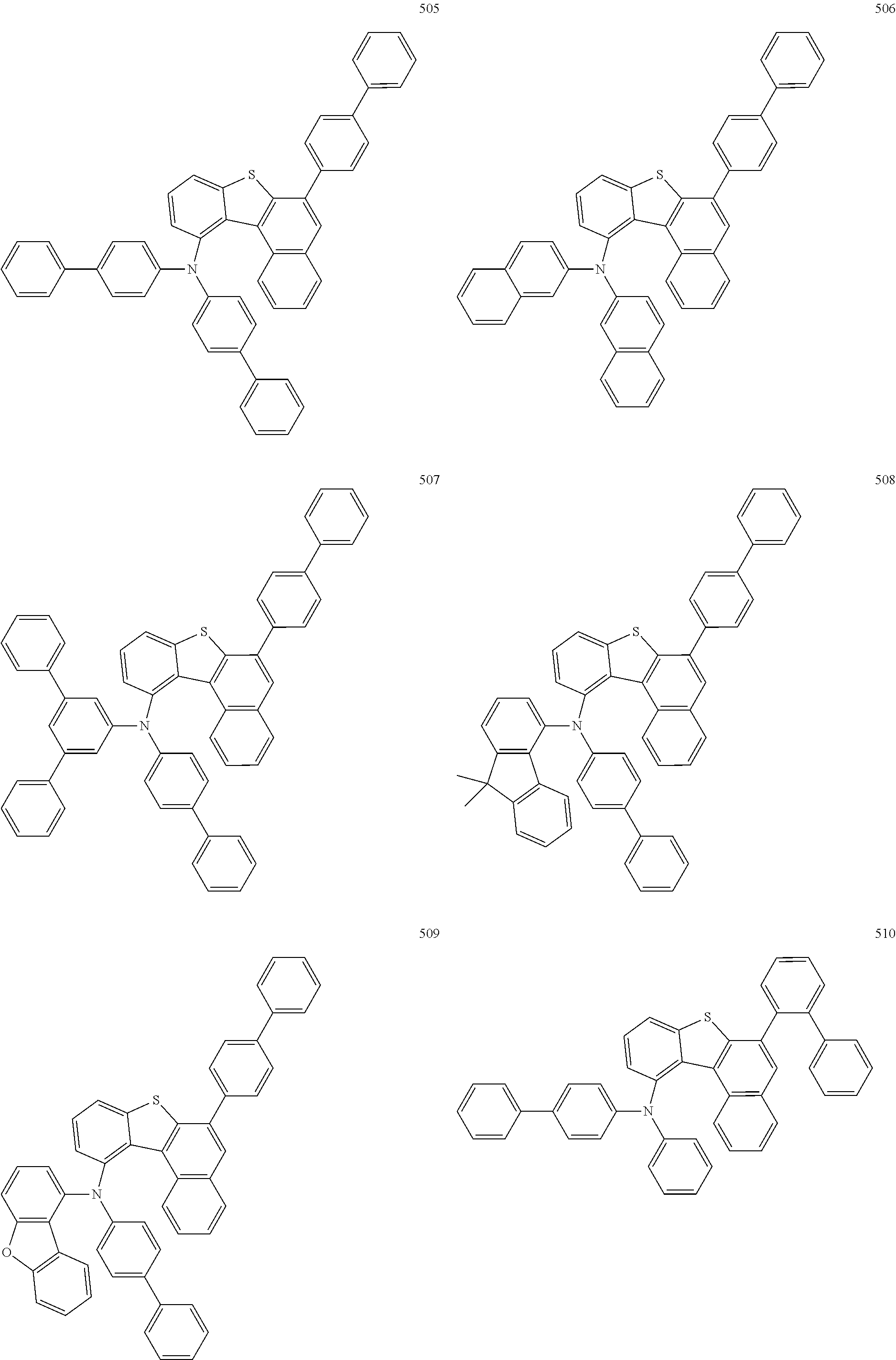

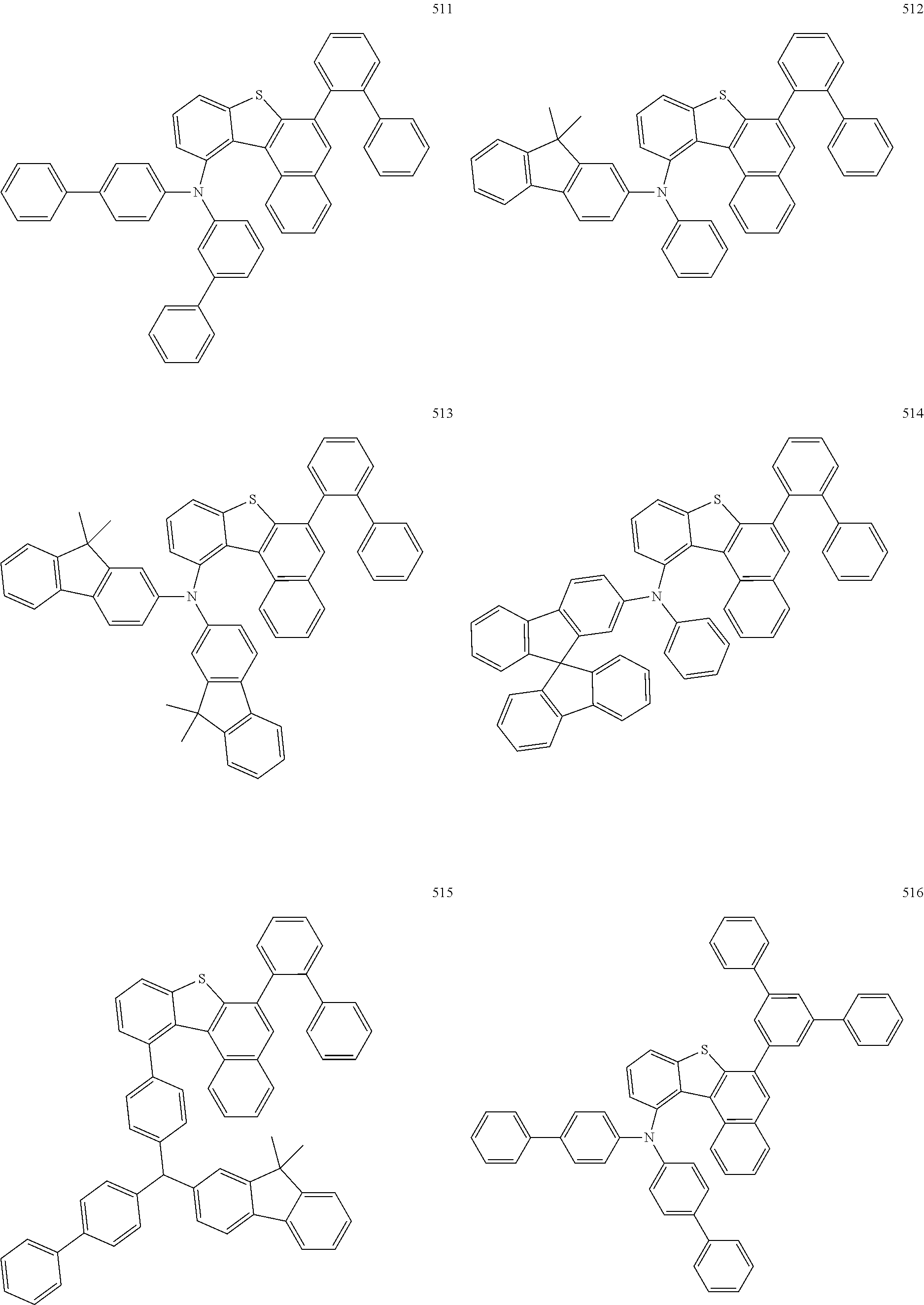

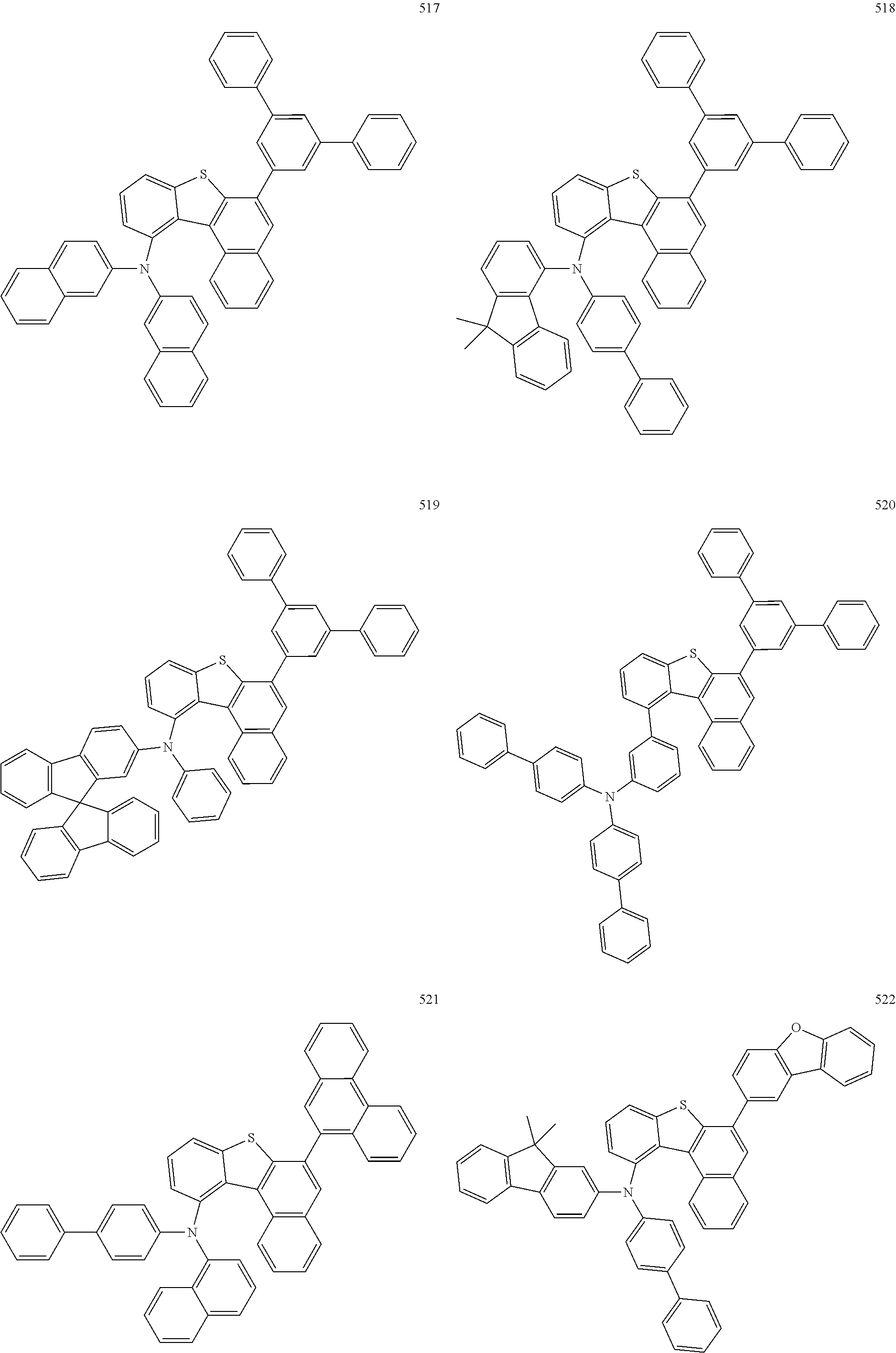

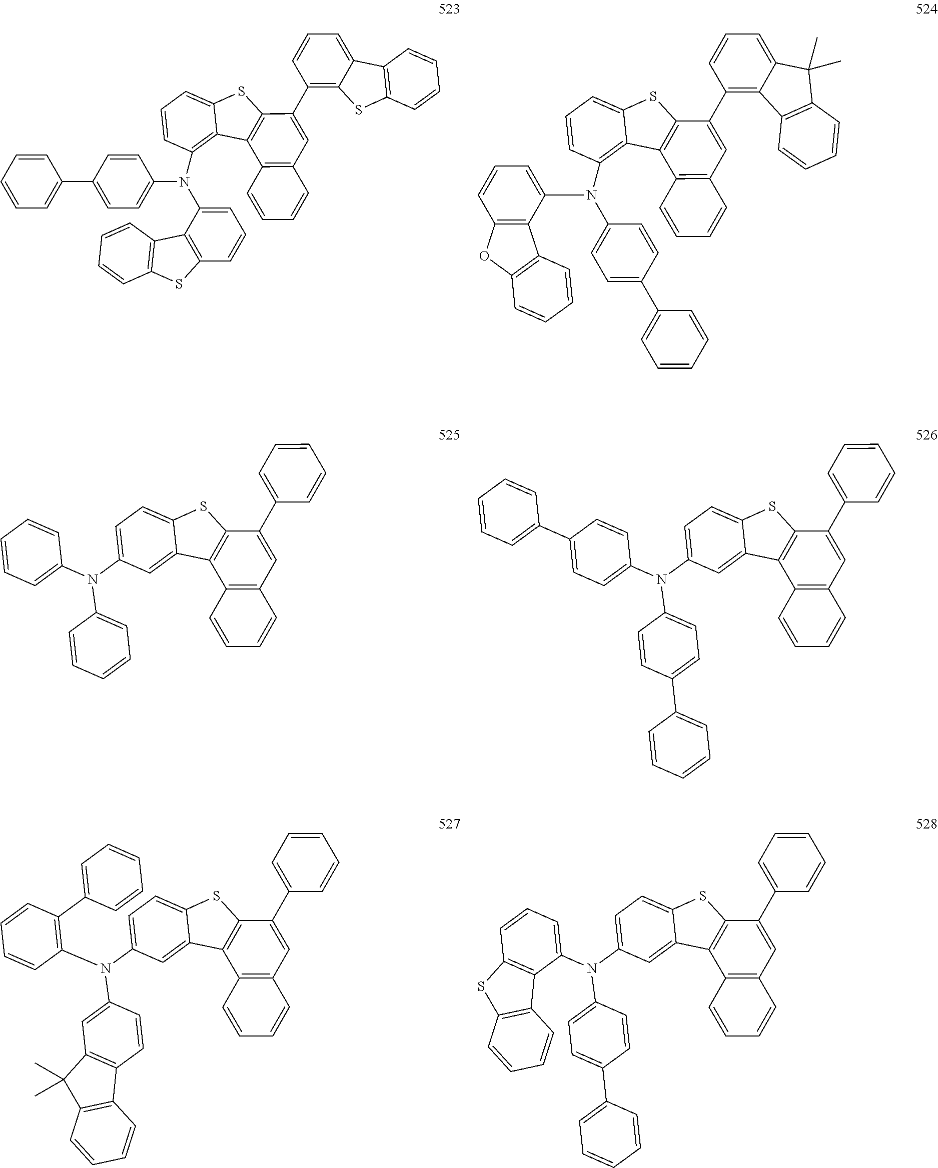 [SMILES: c1ccc(-c2ccc(N(c3cccc4sc5ccccc5c34)c3cccc4sc5c(-c6cccc7c6sc6ccccc67)cc6ccccc6c5c34)cc2)cc1 ; CC1(C)c2ccccc2-c2c(-c3cc4ccccc4c4c3sc3cccc(N(c5ccc(-c6ccccc6)cc5)c5cccc6oc7ccccc7c56)c34)cccc21 ; c1ccc(-c2cc3ccccc3c3c2sc2ccc(N(c4ccccc4)c4ccccc4)cc23)cc1 ; c1ccc(-c2ccc(N(c3ccc(-c4ccccc4)cc3)c3ccc4sc5c(-c6ccccc6)cc6ccccc6c5c4c3)cc2)cc1 ; CC1(C)c2ccccc2-c2ccc(N(c3ccc4sc5c(-c6ccccc6)cc6ccccc6c5c4c3)c3ccccc3-c3ccccc3)cc21 ; c1ccc(-c2ccc(N(c3ccc4sc5c(-c6ccccc6)cc6ccccc6c5c4c3)c3cccc4sc5ccccc5c34)cc2)cc1]

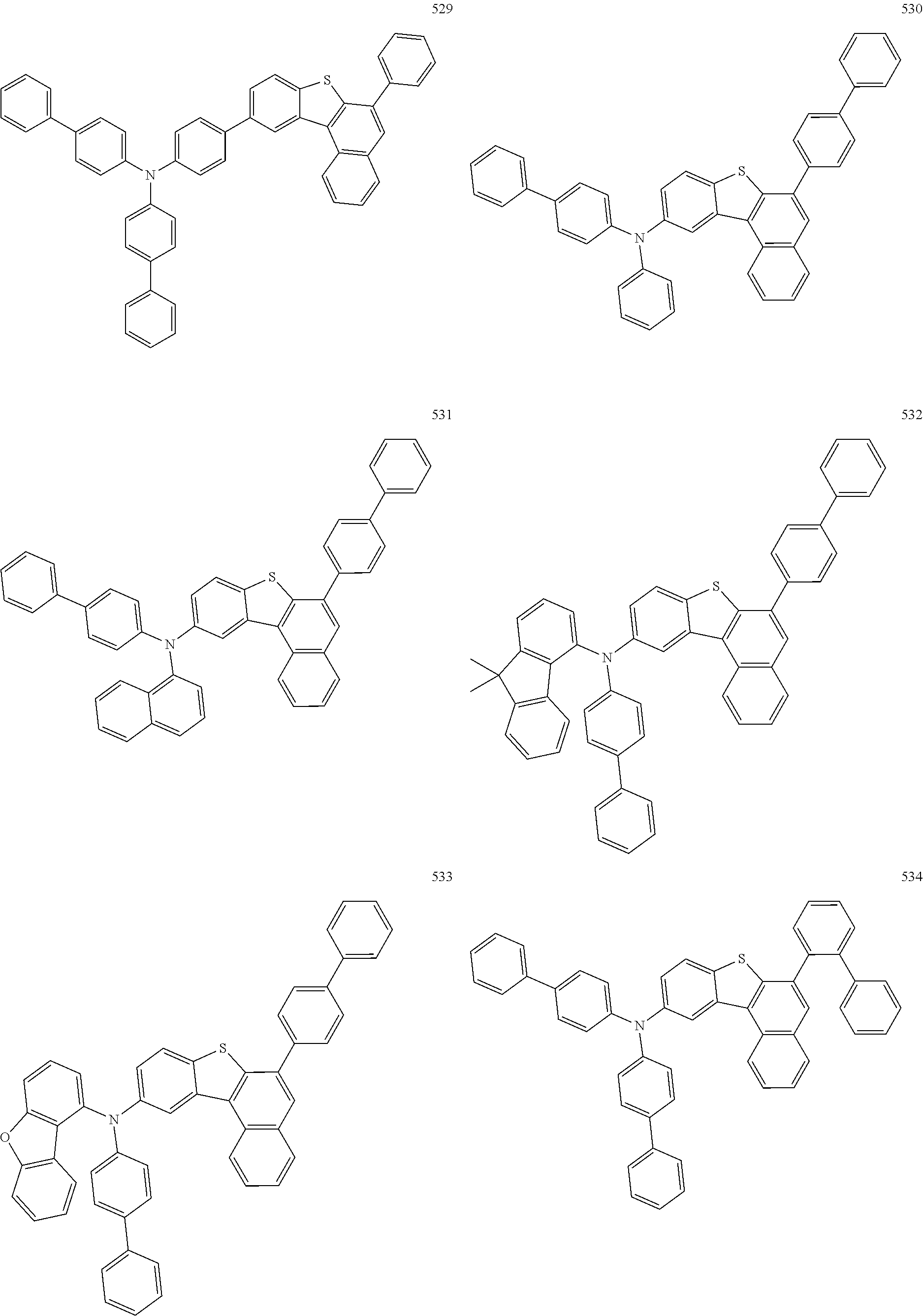

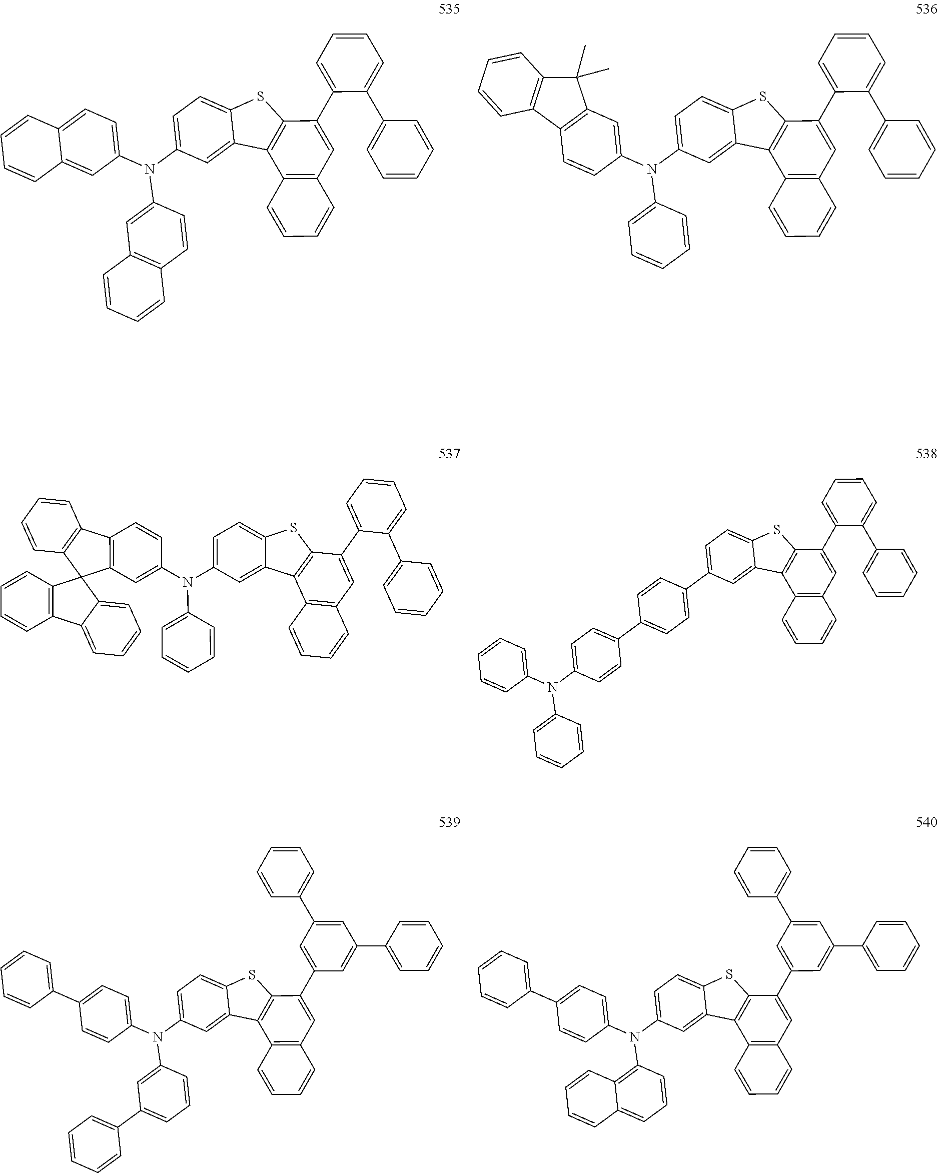

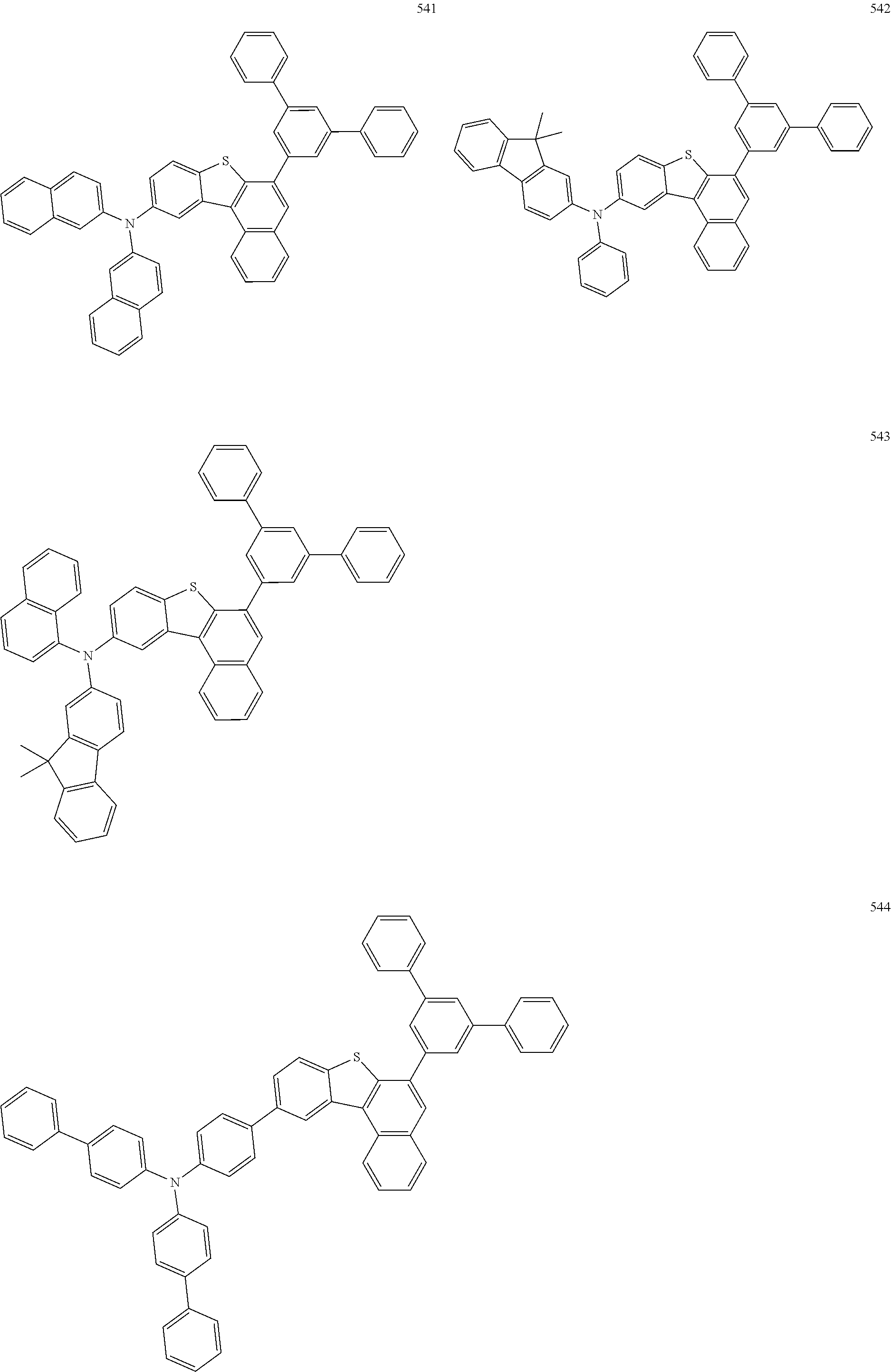

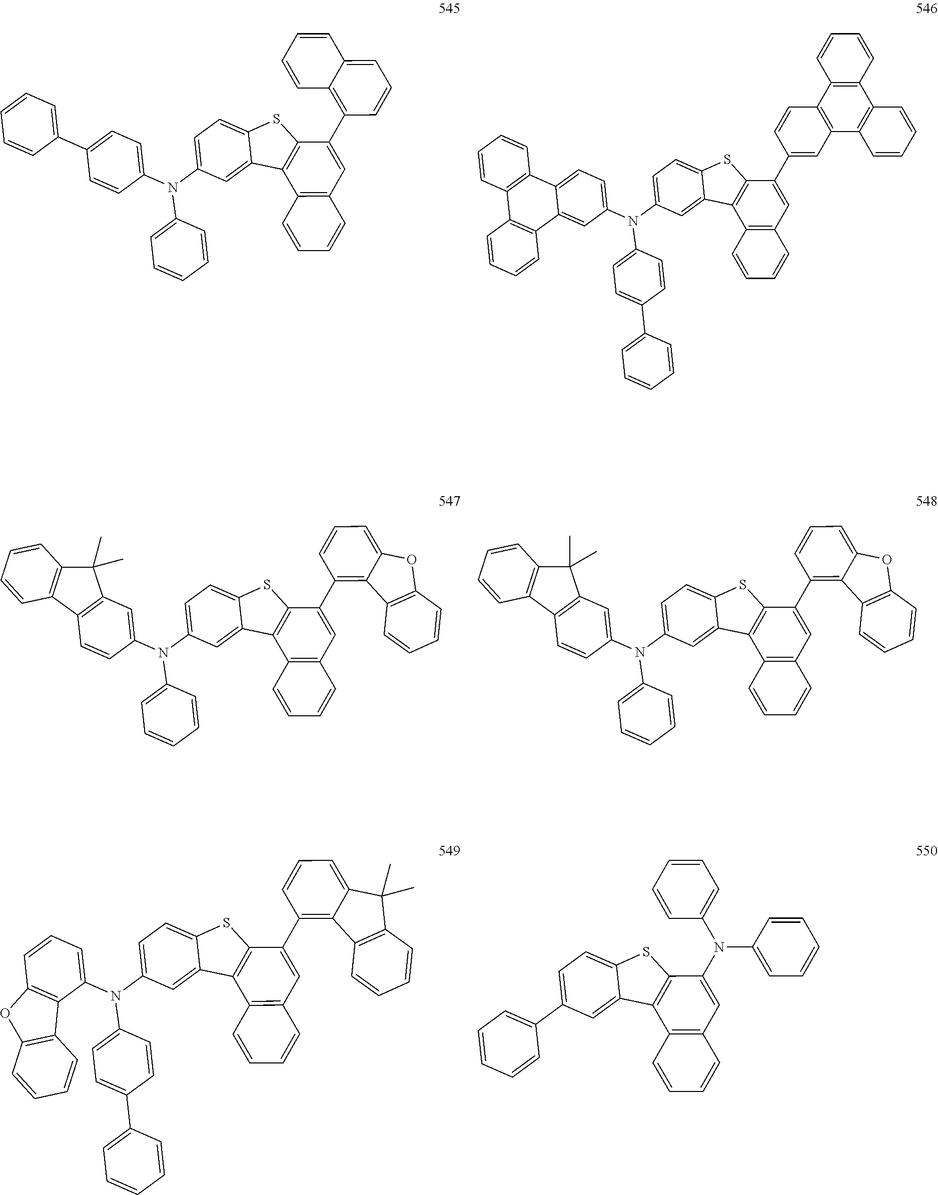

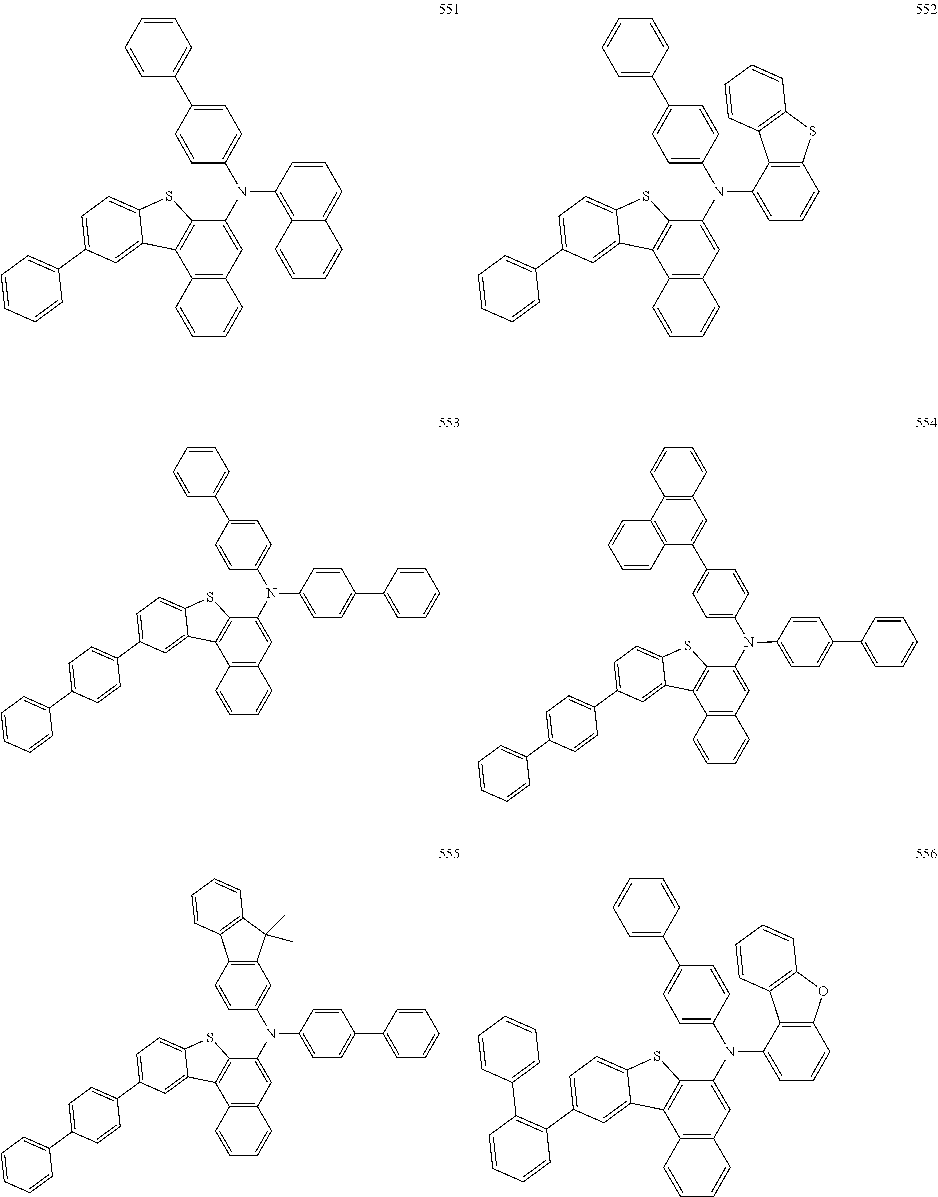

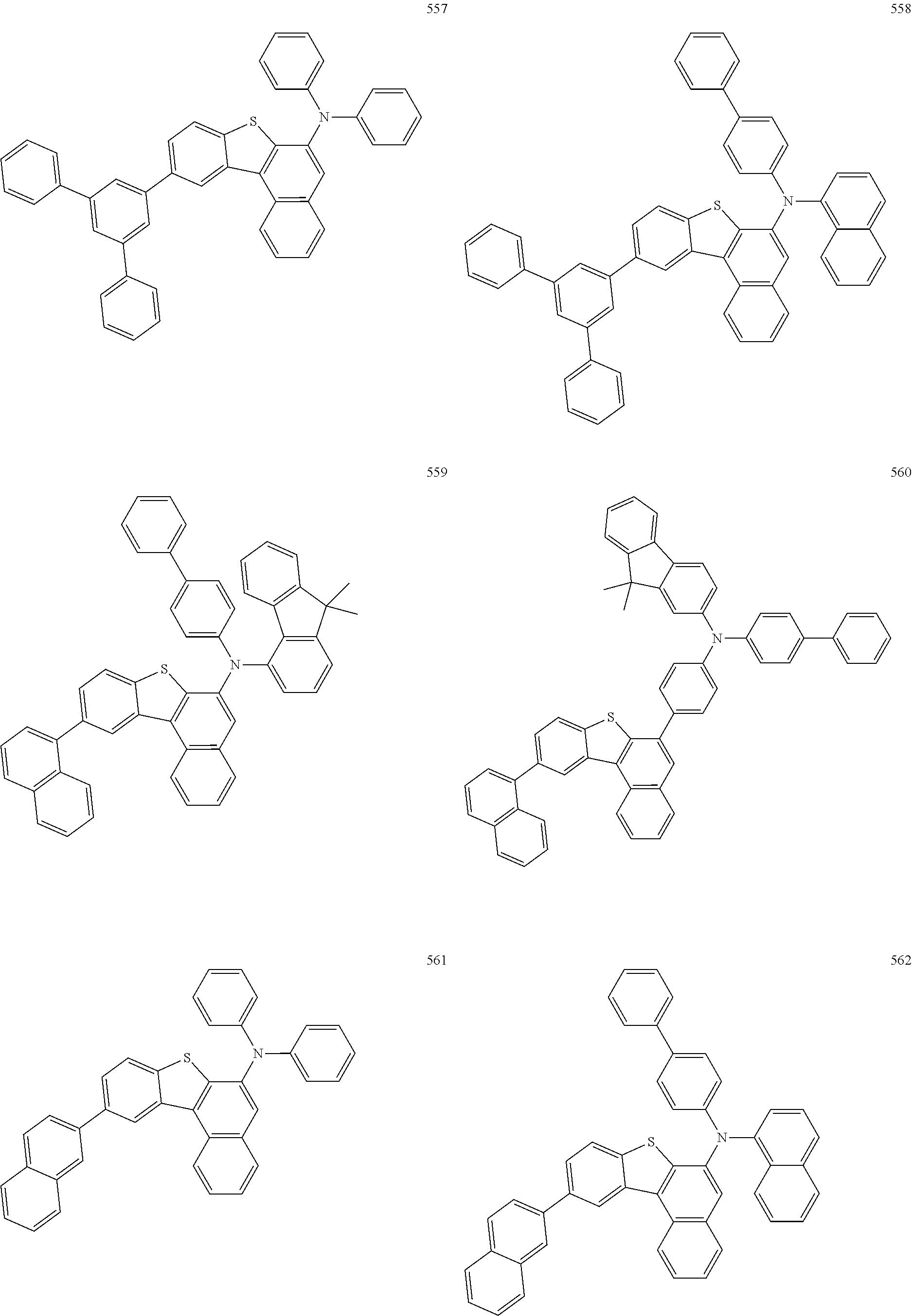

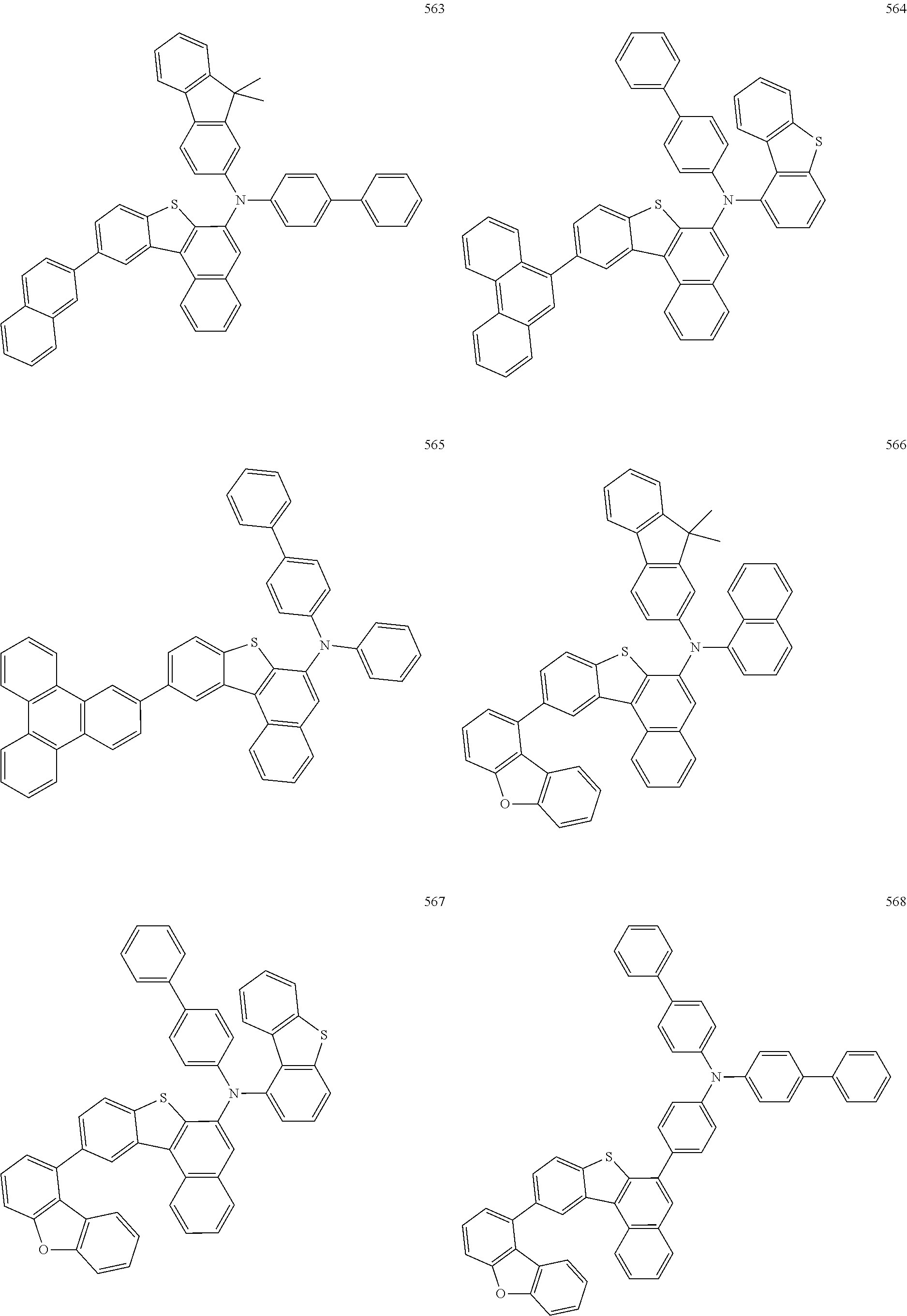

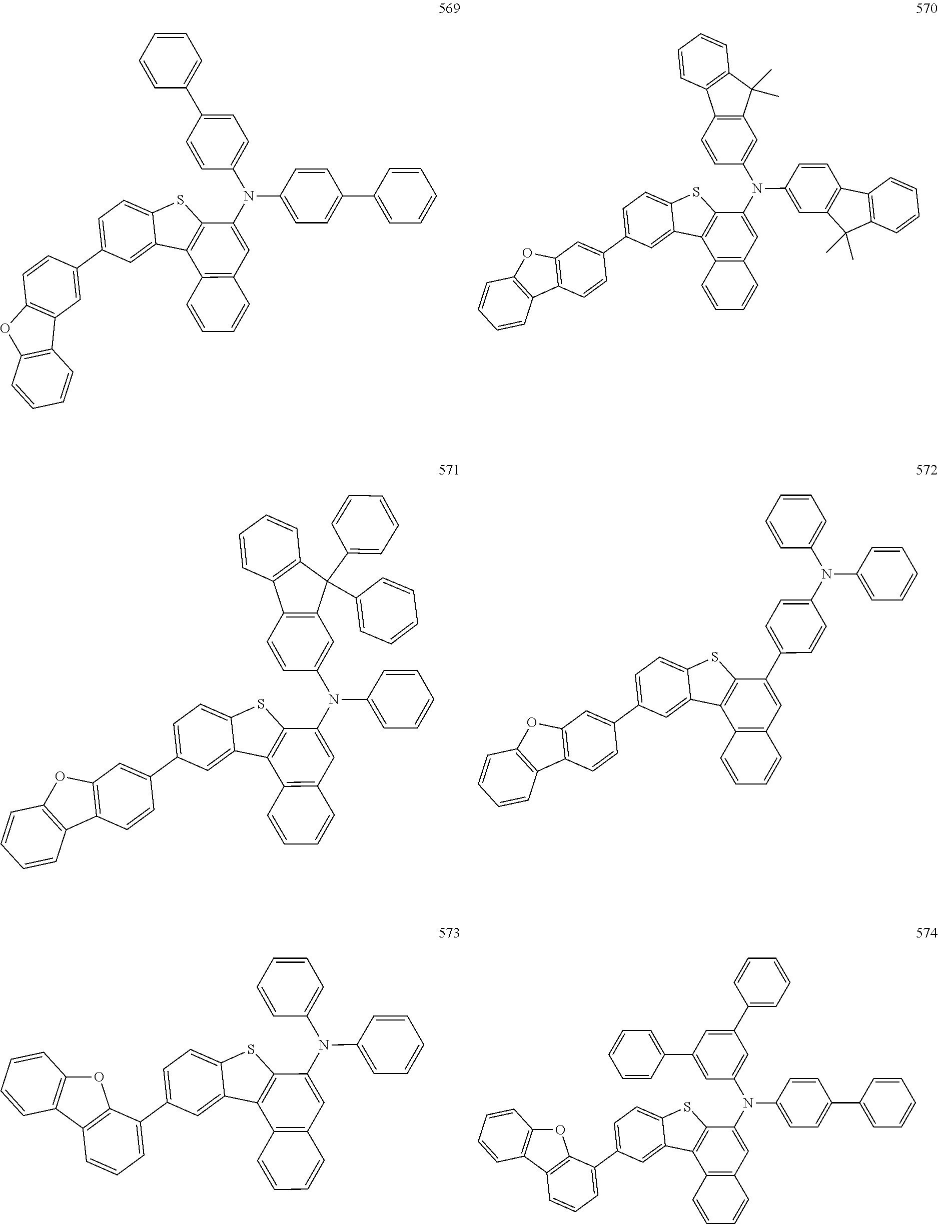

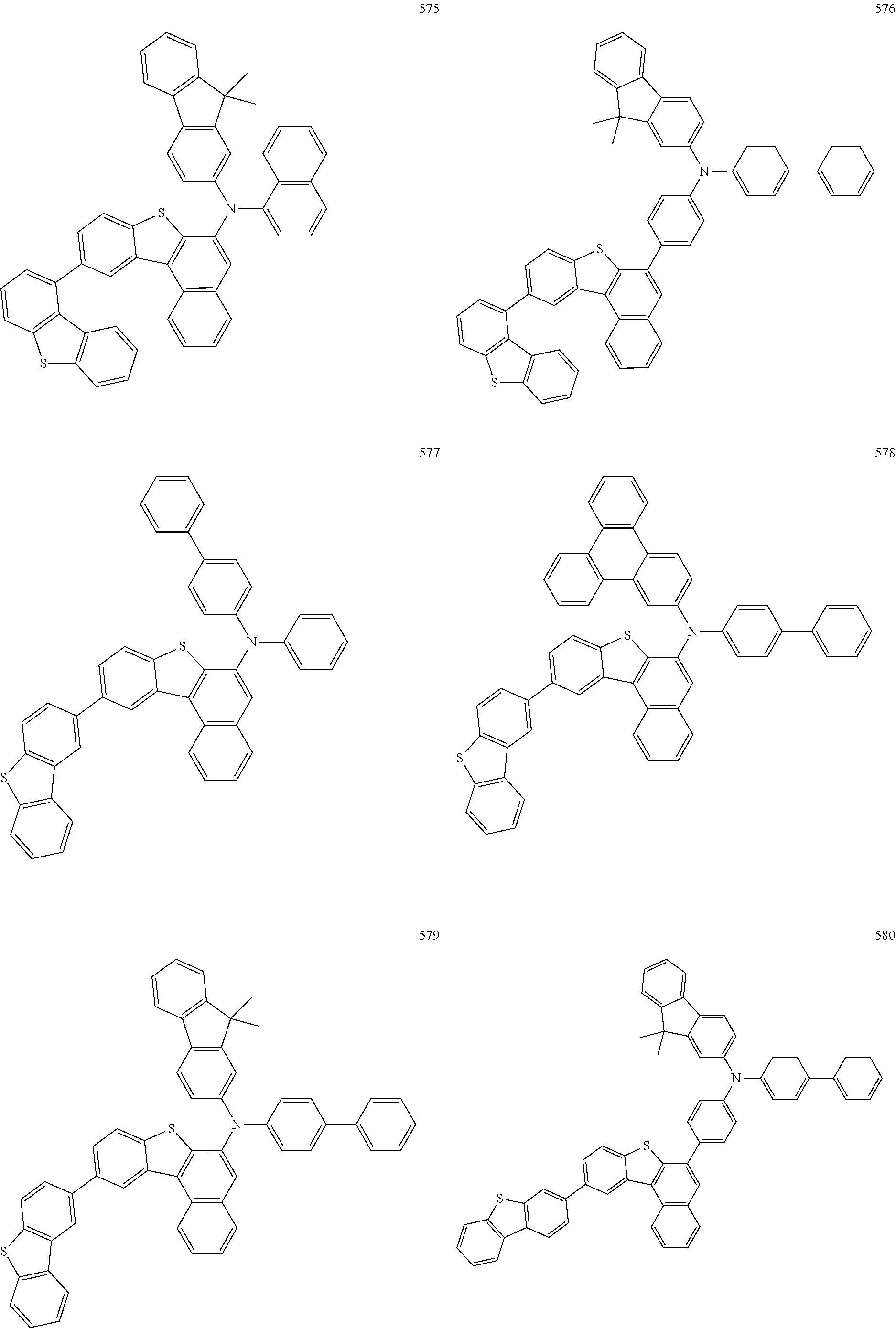

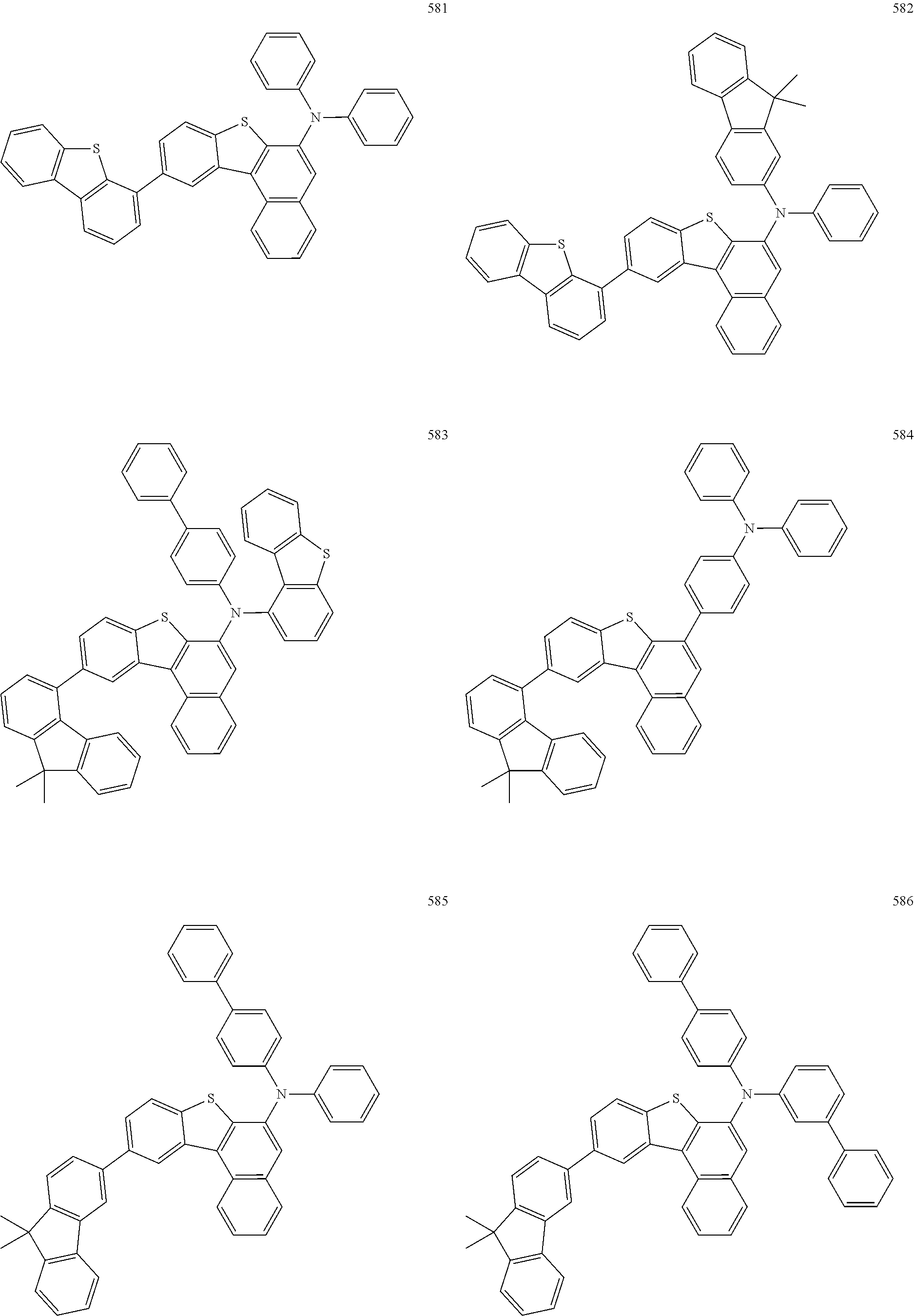

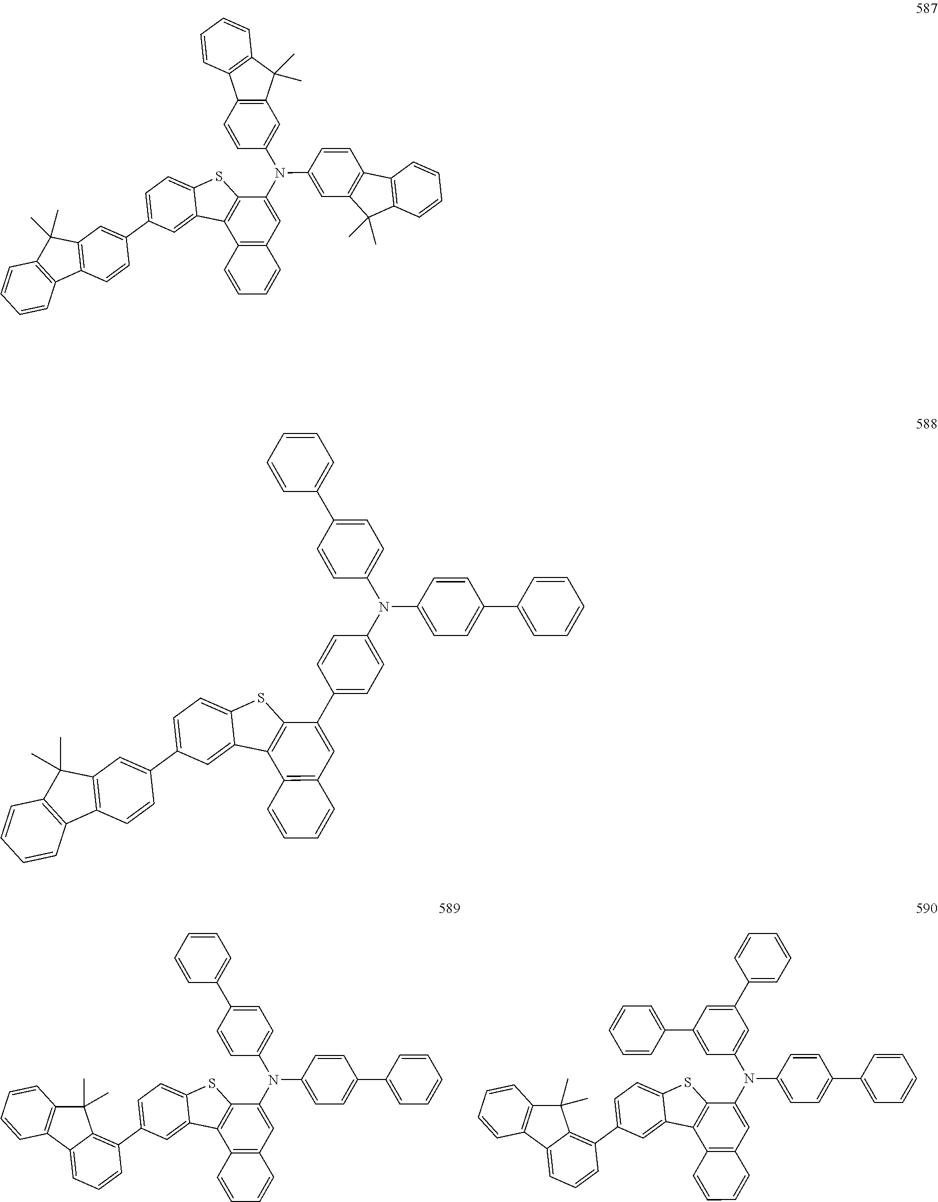 [SMILES: CC1(C)c2ccccc2-c2ccc(-c3ccc4sc5c(N(c6ccc7c(c6)C(C)(C)c6ccccc6-7)c6ccc7c(c6)C(C)(C)c6ccccc6-7)cc6ccccc6c5c4c3)cc21 ; CC1(C)c2ccccc2-c2ccc(-c3ccc4sc5c(-c6ccc(N(c7ccc(-c8ccccc8)cc7)c7ccc(-c8ccccc8)cc7)cc6)cc6ccccc6c5c4c3)cc21 ; CC1(C)c2ccccc2-c2cccc(-c3ccc4sc5c(N(c6ccc(-c7ccccc7)cc6)c6ccc(-c7ccccc7)cc6)cc6ccccc6c5c4c3)c21 ; CC1(C)c2ccccc2-c2cccc(-c3ccc4sc5c(N(c6ccc(-c7ccccc7)cc6)c6cc(-c7ccccc7)cc(-c7ccccc7)c6)cc6ccccc6c5c4c3)c21]

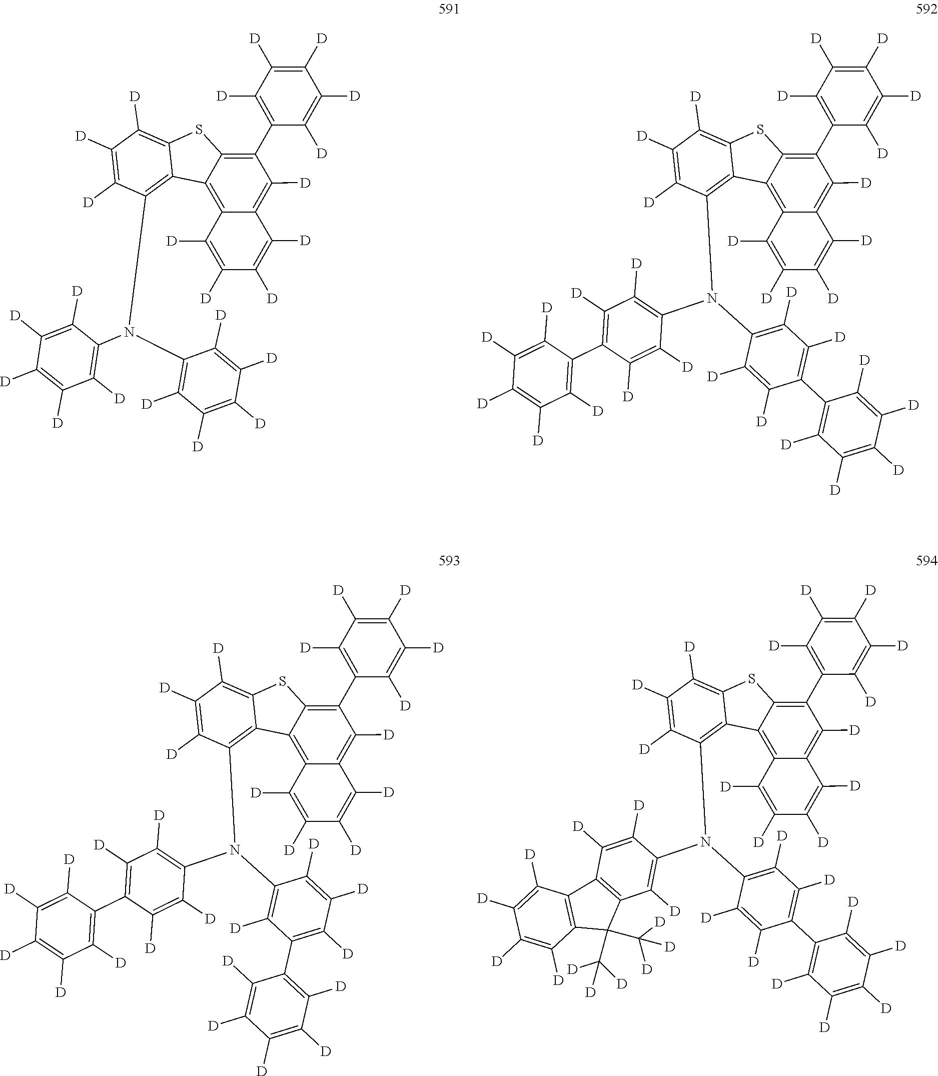

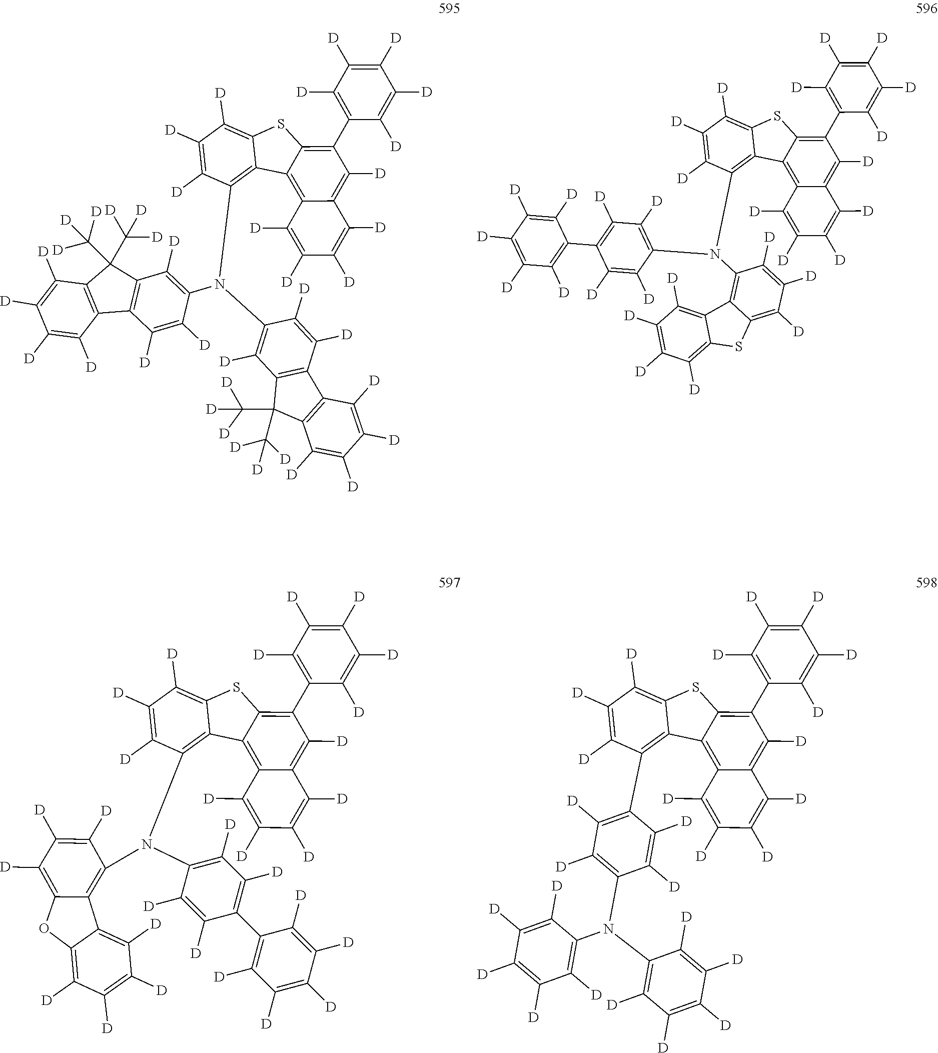

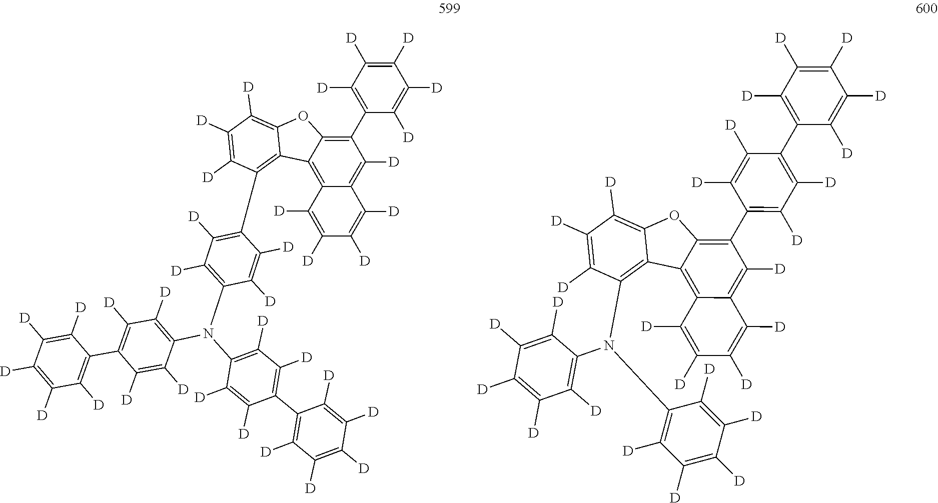

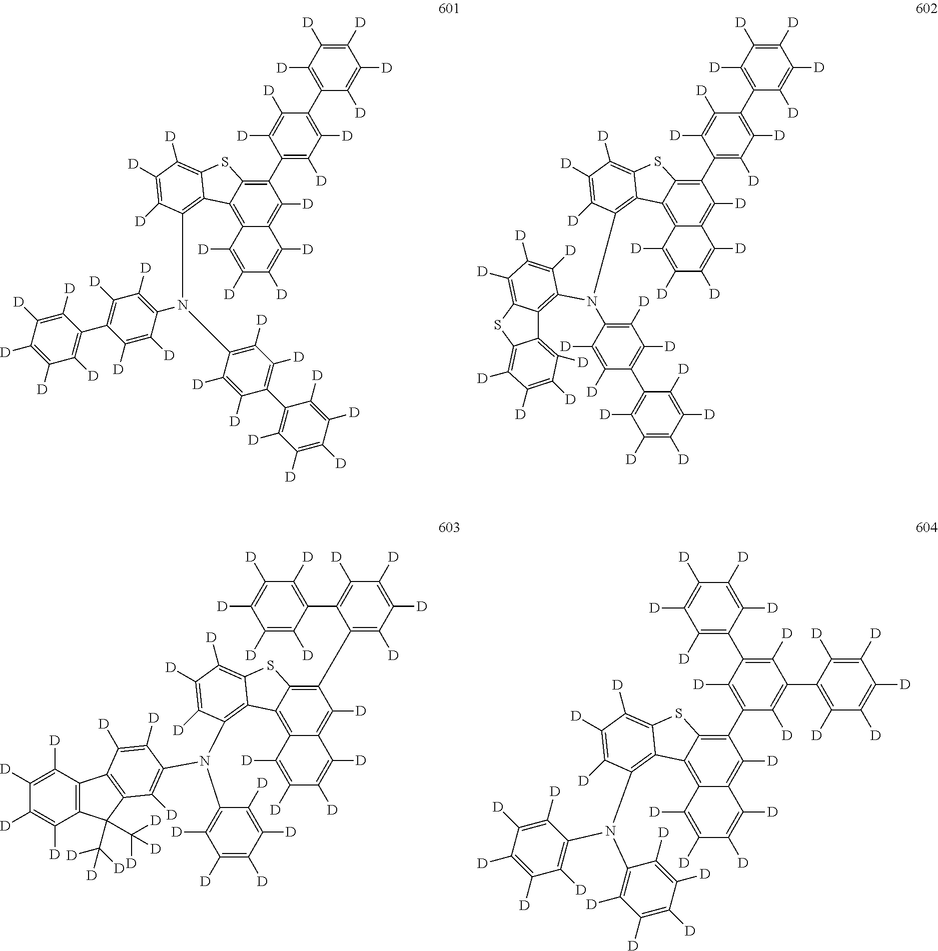

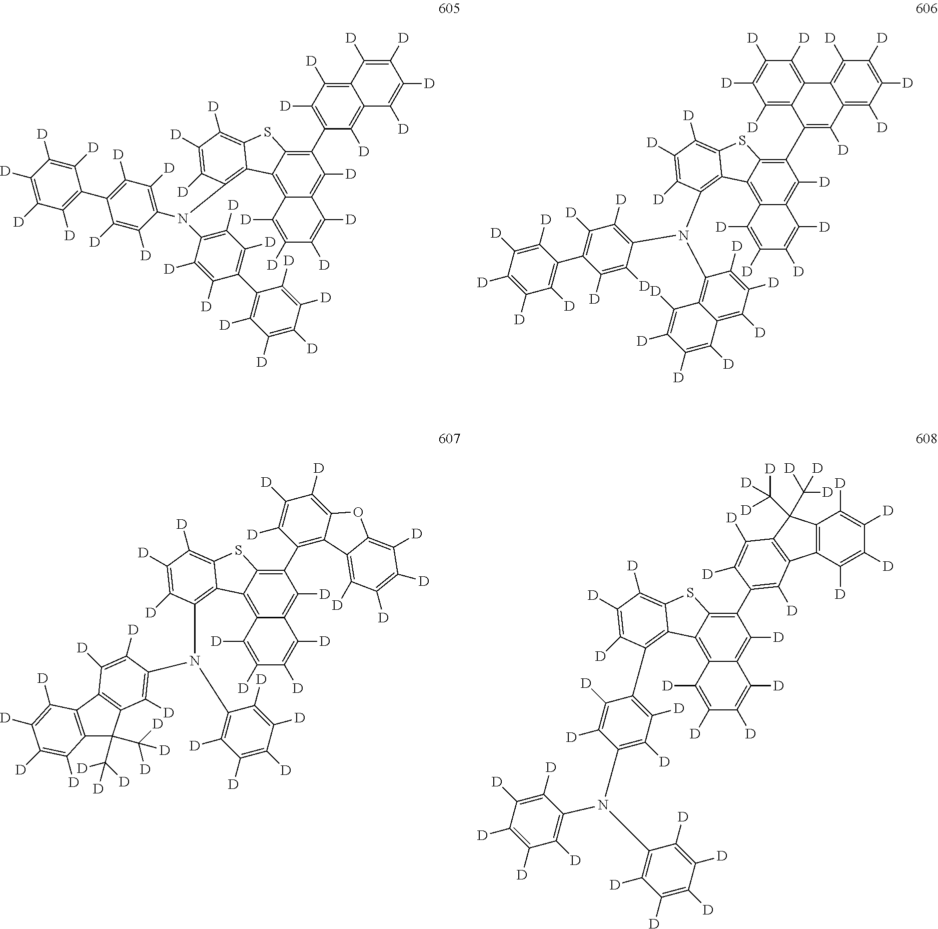

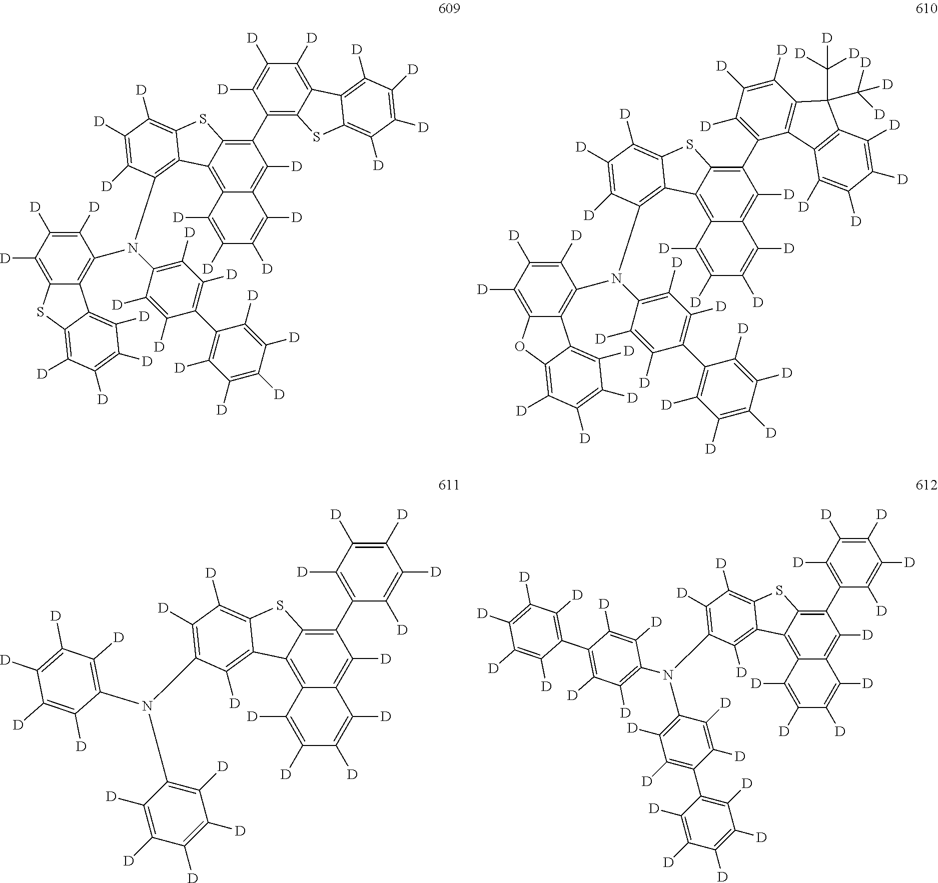

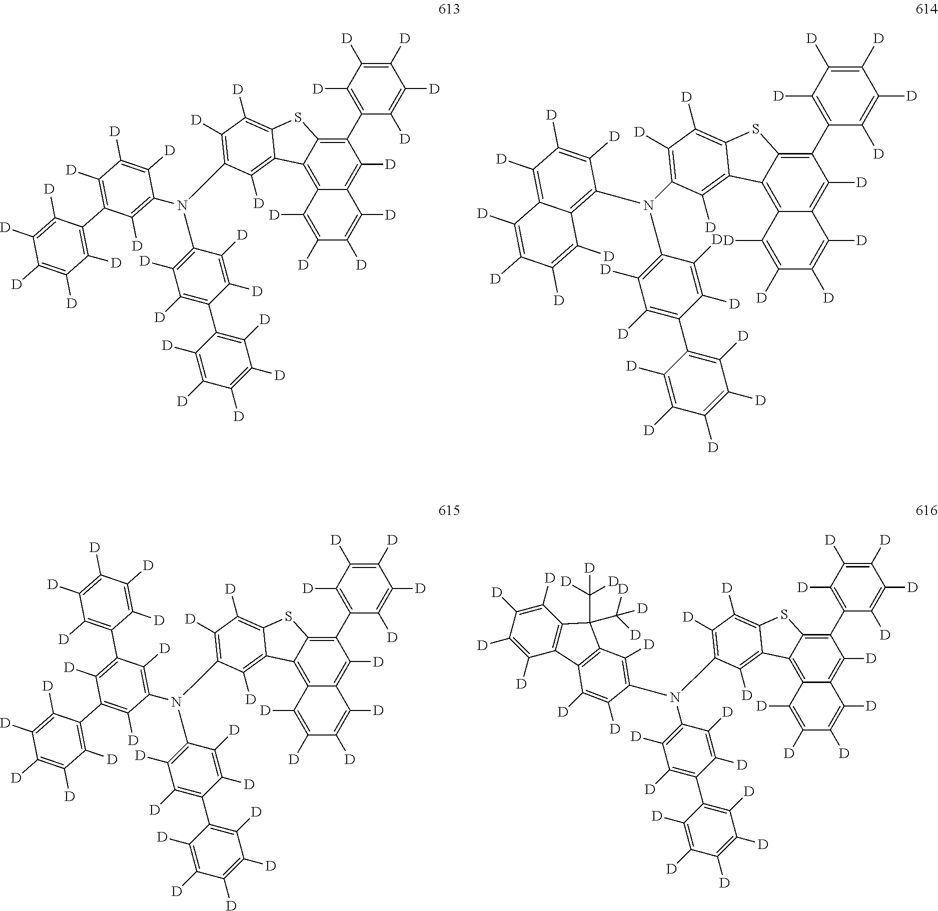

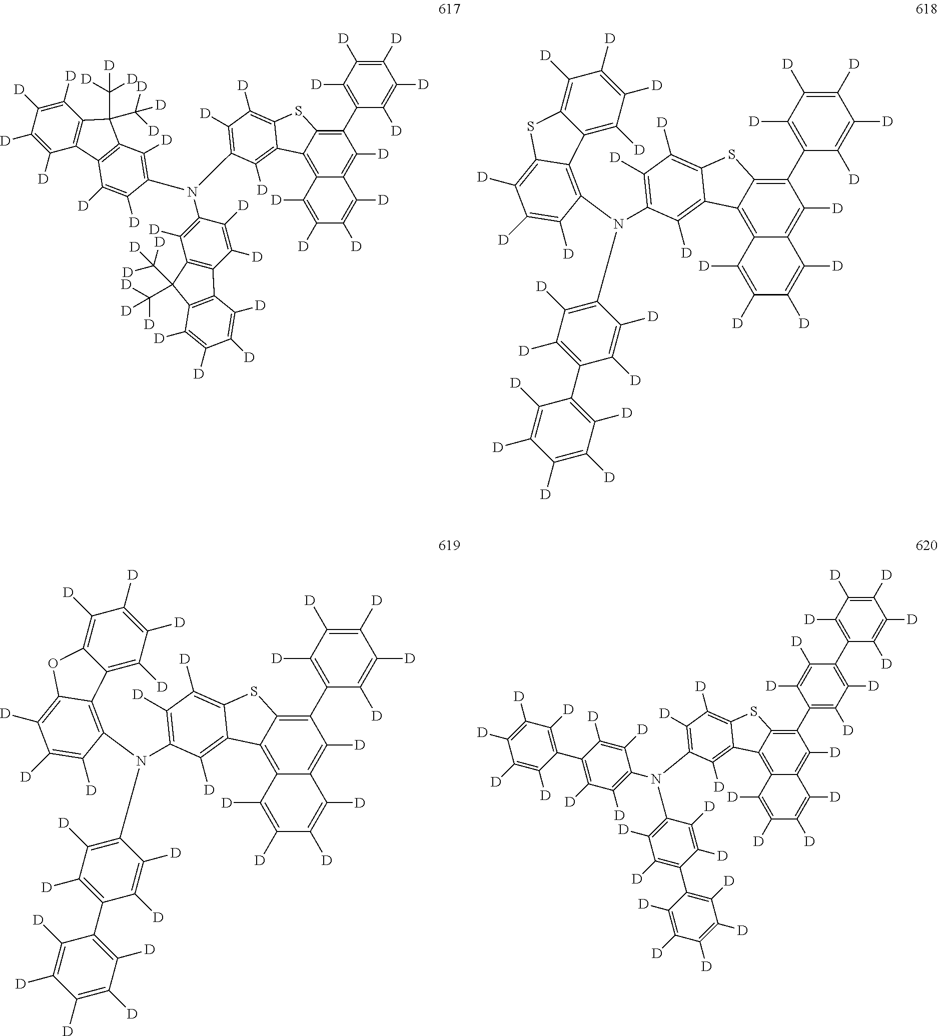

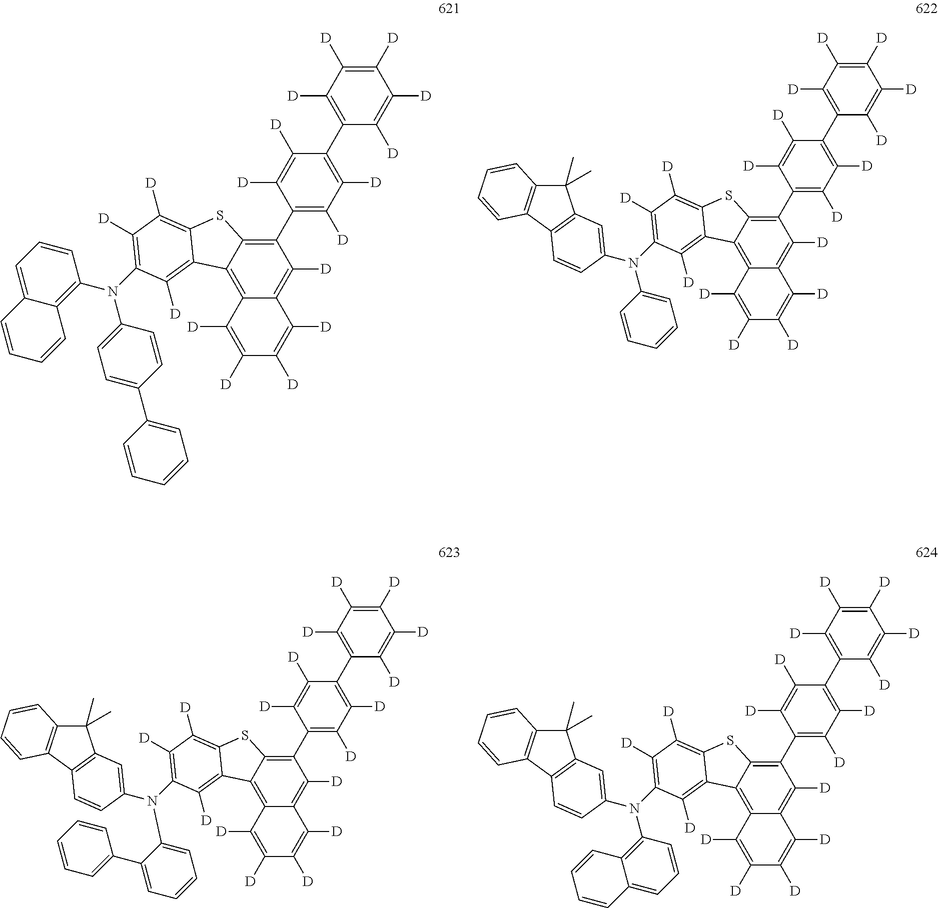

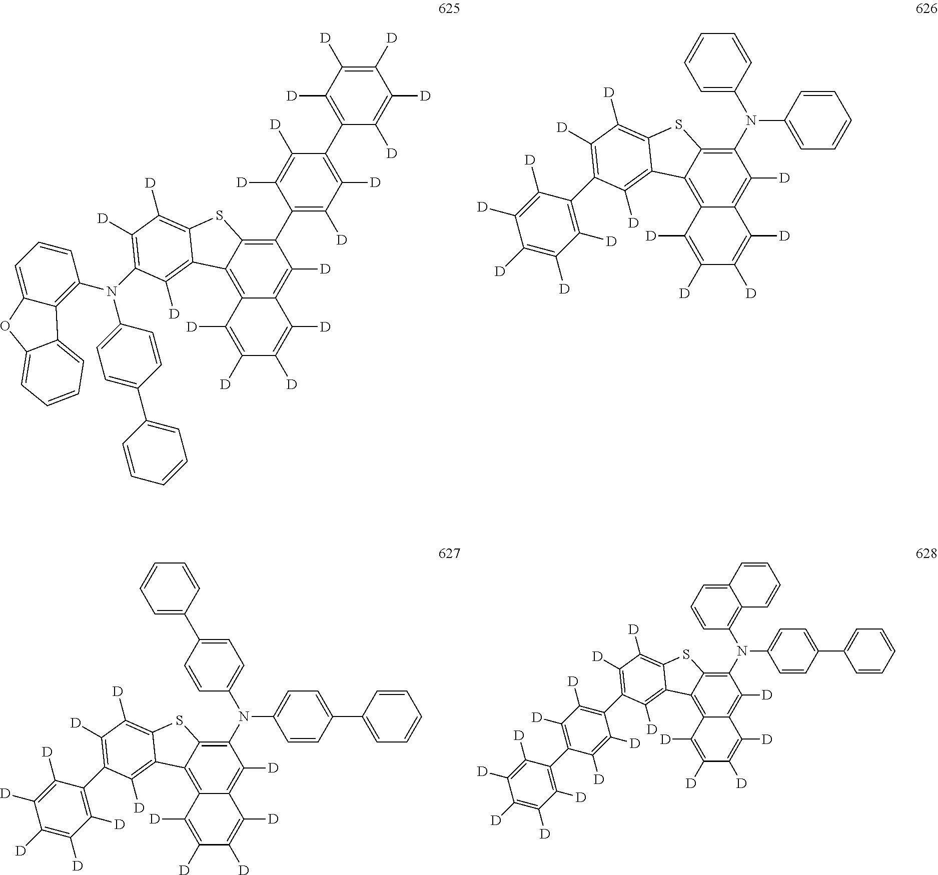

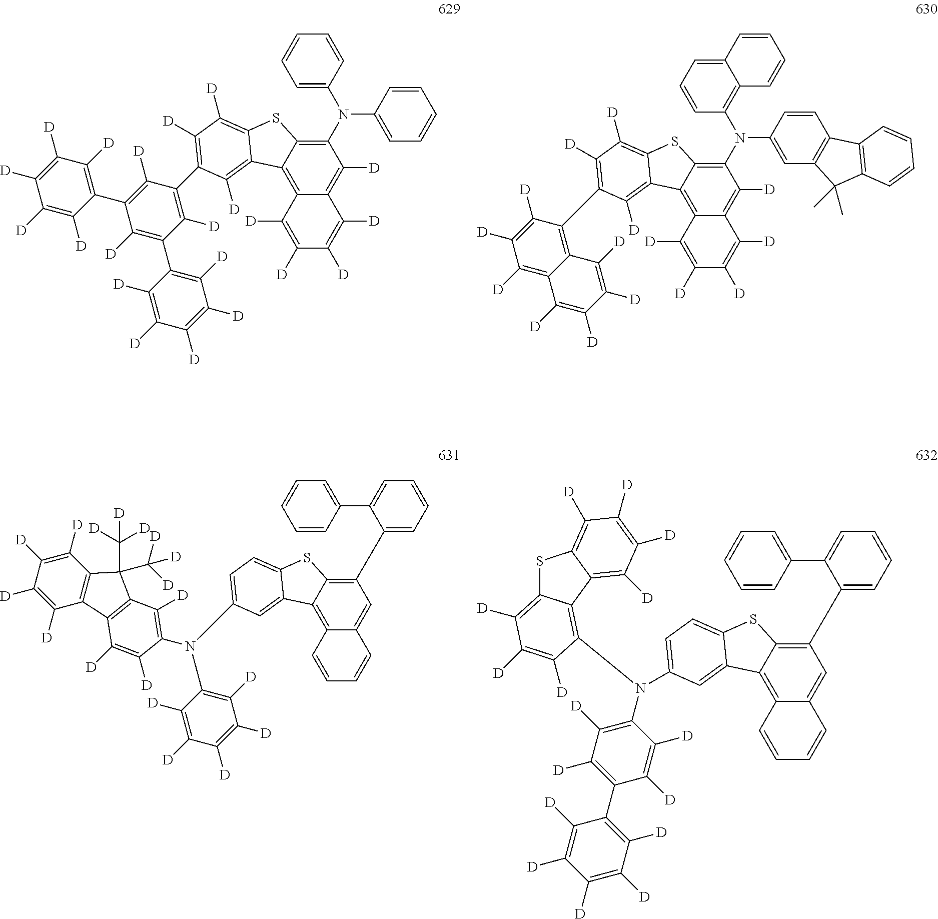

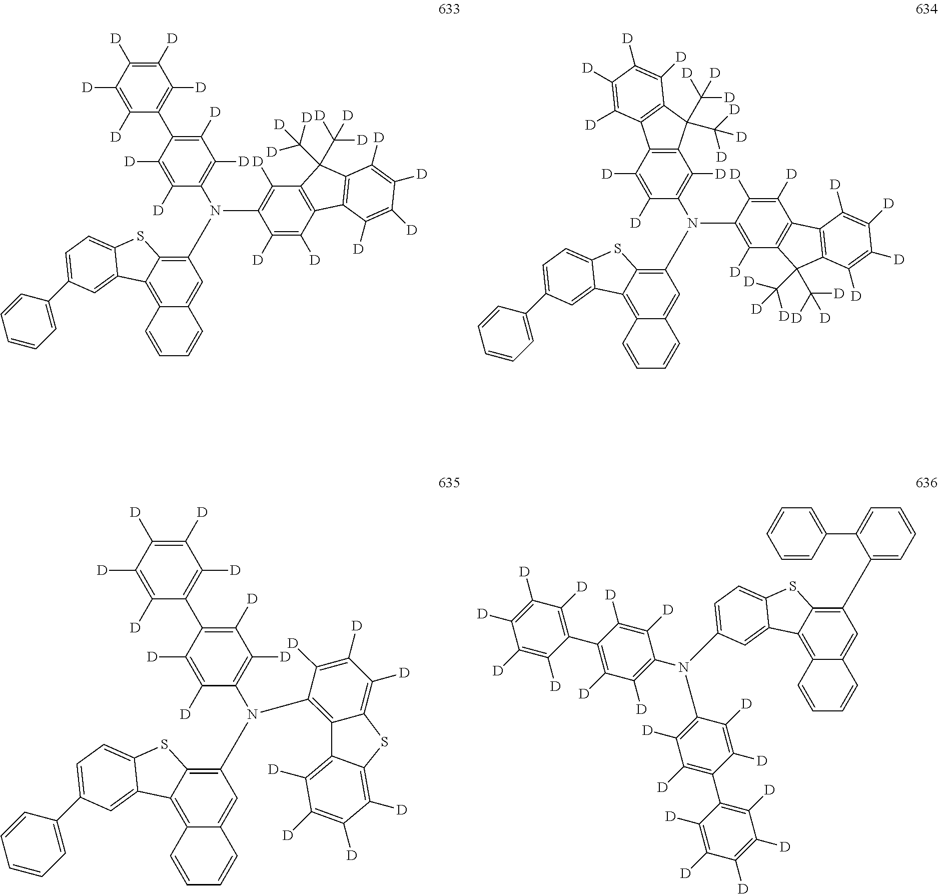

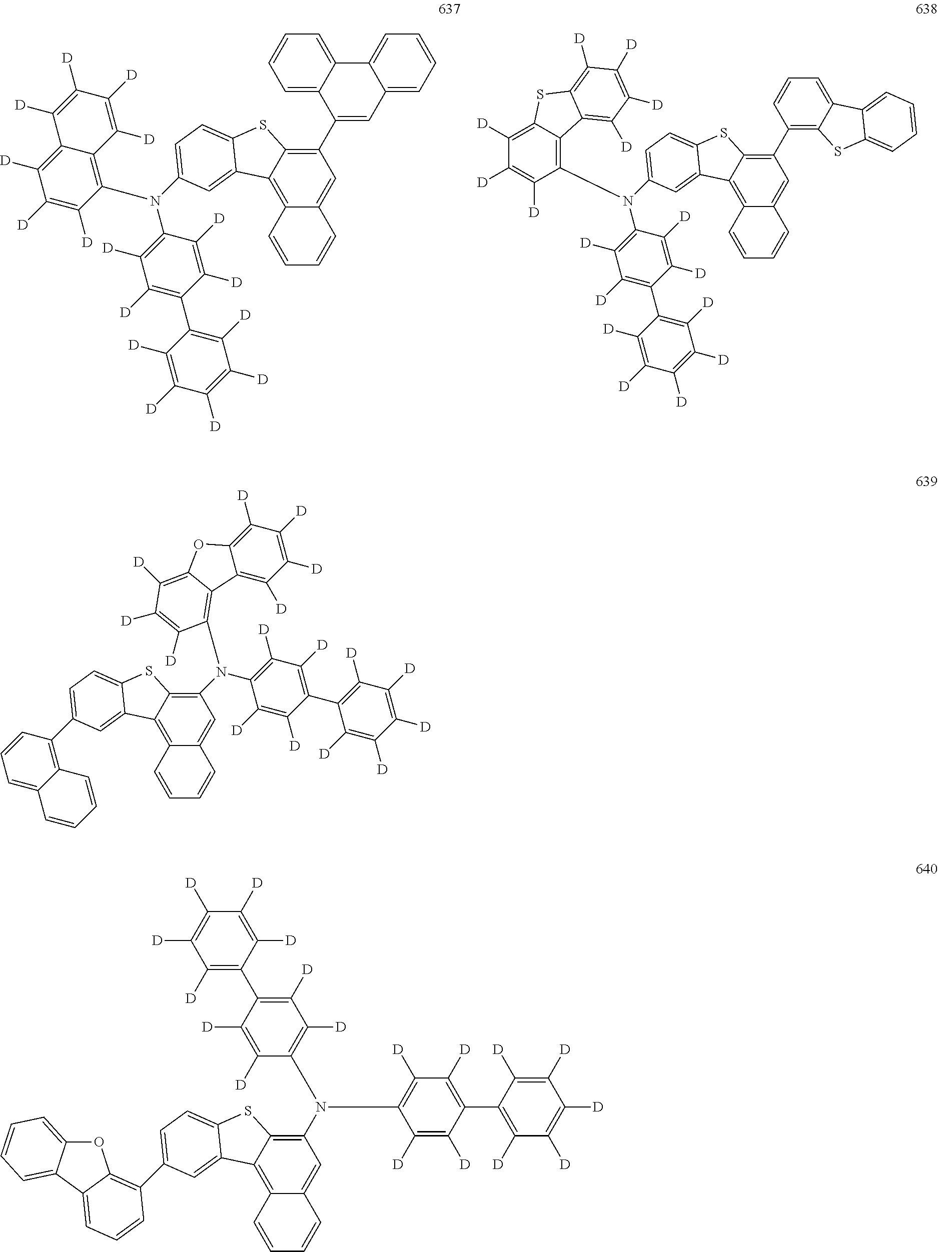

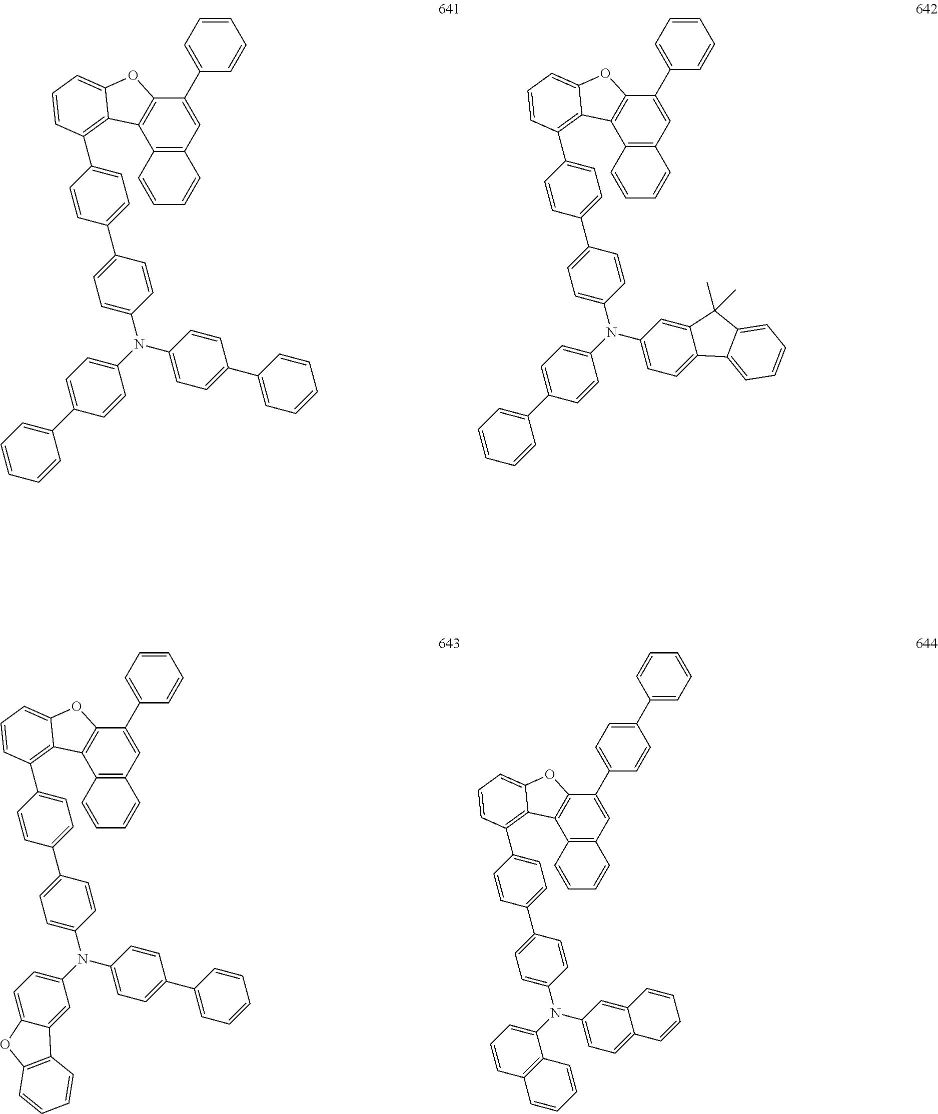 [SMILES: c1ccc(-c2ccc(N(c3ccc(-c4ccccc4)cc3)c3ccc(-c4ccc(-c5cccc6oc7c(-c8ccccc8)cc8ccccc8c7c56)cc4)cc3)cc2)cc1 ; CC1(C)c2ccccc2-c2ccc(N(c3ccc(-c4ccccc4)cc3)c3ccc(-c4ccc(-c5cccc6oc7c(-c8ccccc8)cc8ccccc8c7c56)cc4)cc3)cc21 ; c1ccc(-c2ccc(N(c3ccc(-c4ccc(-c5cccc6oc7c(-c8ccccc8)cc8ccccc8c7c56)cc4)cc3)c3ccc4oc5ccccc5c4c3)cc2)cc1 ; c1ccc(-c2ccc(-c3cc4ccccc4c4c3oc3cccc(-c5ccc(-c6ccc(N(c7ccc8ccccc8c7)c7cccc8ccccc78)cc6)cc5)c34)cc2)cc1]

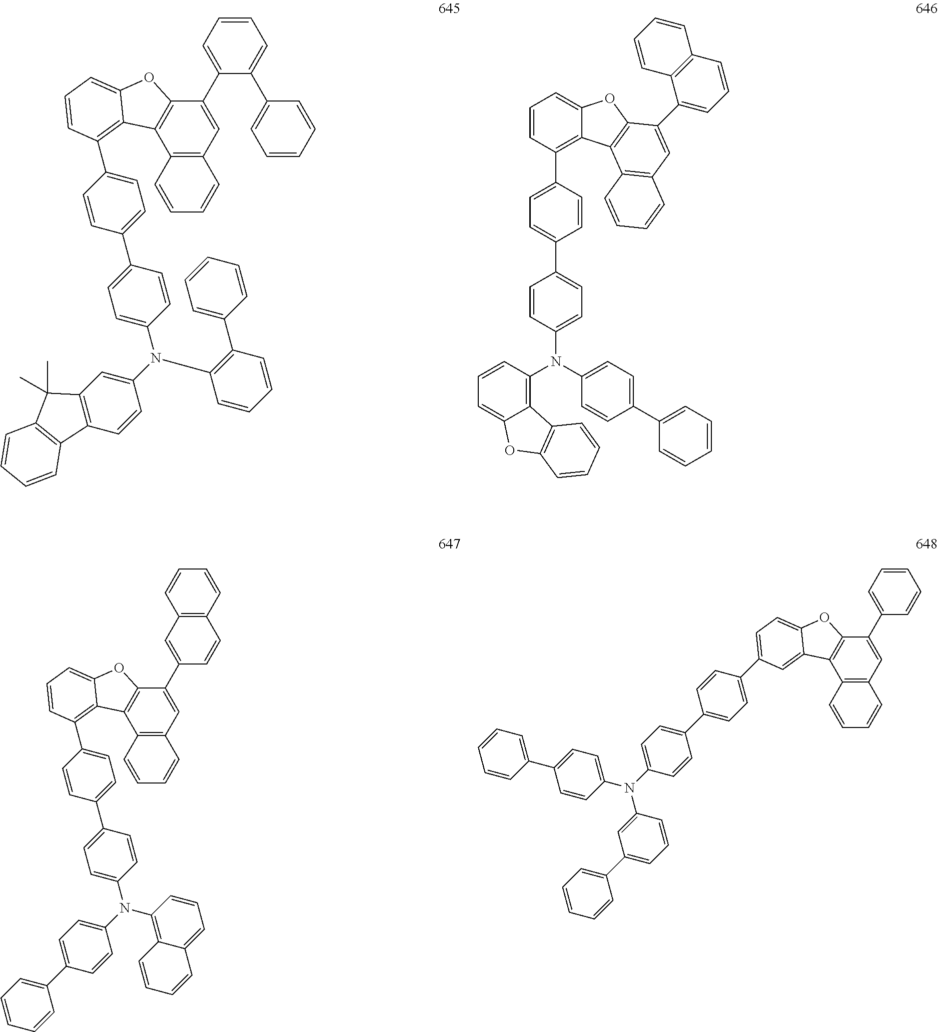 [SMILES: CC1(C)c2ccccc2-c2ccc(N(c3ccc(-c4ccc(-c5cccc6oc7c(-c8ccccc8-c8ccccc8)cc8ccccc8c7c56)cc4)cc3)c3ccccc3-c3ccccc3)cc21 ; c1ccc(-c2ccc(N(c3ccc(-c4ccc(-c5cccc6oc7c(-c8cccc9ccccc89)cc8ccccc8c7c56)cc4)cc3)c3cccc4oc5ccccc5c34)cc2)cc1 ; c1ccc(-c2ccc(N(c3ccc(-c4ccc(-c5cccc6oc7c(-c8ccc9ccccc9c8)cc8ccccc8c7c56)cc4)cc3)c3cccc4ccccc34)cc2)cc1 ; c1ccc(-c2ccc(N(c3ccc(-c4ccc(-c5ccc6oc7c(-c8ccccc8)cc8ccccc8c7c6c5)cc4)cc3)c3cccc(-c4ccccc4)c3)cc2)cc1]

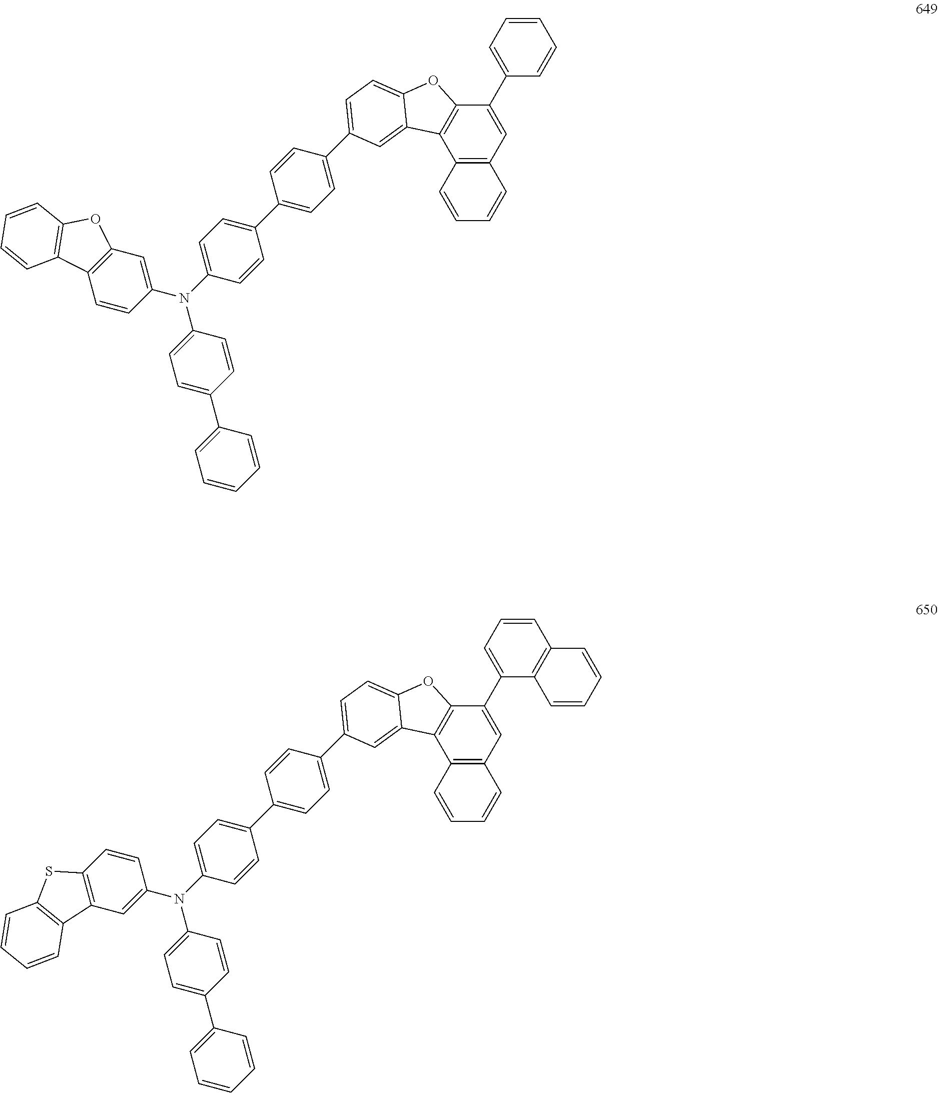 [SMILES: c1ccc(-c2ccc(N(c3ccc(-c4ccc(-c5ccc6oc7c(-c8ccccc8)cc8ccccc8c7c6c5)cc4)cc3)c3ccc4c(c3)oc3ccccc34)cc2)cc1 ; c1ccc(-c2ccc(N(c3ccc(-c4ccc(-c5ccc6oc7c(-c8cccc9ccccc89)cc8ccccc8c7c6c5)cc4)cc3)c3ccc4sc5ccccc5c4c3)cc2)cc1]

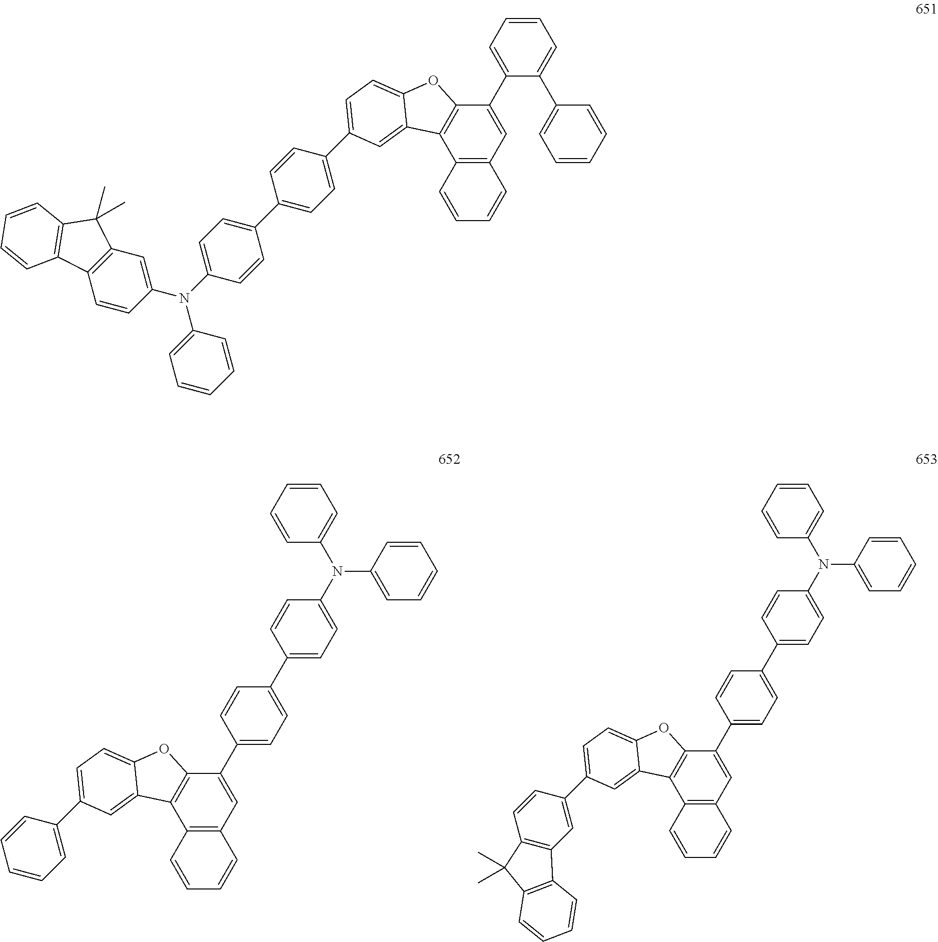 [SMILES: CC1(C)c2ccccc2-c2ccc(N(c3ccccc3)c3ccc(-c4ccc(-c5ccc6oc7c(-c8ccccc8-c8ccccc8)cc8ccccc8c7c6c5)cc4)cc3)cc21 ; c1ccc(-c2ccc3oc4c(-c5ccc(-c6ccc(N(c7ccccc7)c7ccccc7)cc6)cc5)cc5ccccc5c4c3c2)cc1 ; CC1(C)c2ccccc2-c2cc(-c3ccc4oc5c(-c6ccc(-c7ccc(N(c8ccccc8)c8ccccc8)cc7)cc6)cc6ccccc6c5c4c3)ccc21]

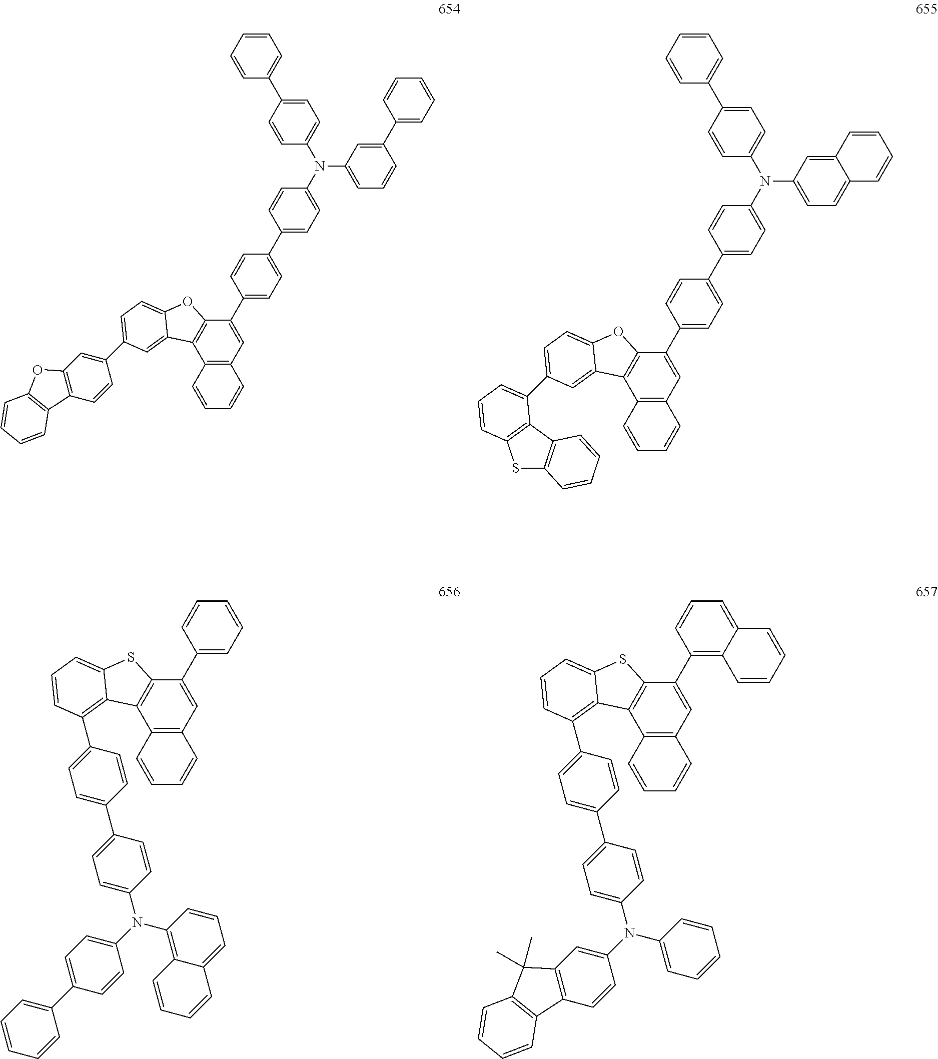 [SMILES: c1ccc(-c2ccc(N(c3ccc(-c4ccc(-c5cc6ccccc6c6c5oc5ccc(-c7ccc8c(c7)oc7ccccc78)cc56)cc4)cc3)c3cccc(-c4ccccc4)c3)cc2)cc1 ; c1ccc(-c2ccc(N(c3ccc(-c4ccc(-c5cc6ccccc6c6c5oc5ccc(-c7cccc8sc9ccccc9c78)cc56)cc4)cc3)c3ccc4ccccc4c3)cc2)cc1 ; c1ccc(-c2ccc(N(c3ccc(-c4ccc(-c5cccc6sc7c(-c8ccccc8)cc8ccccc8c7c56)cc4)cc3)c3cccc4ccccc34)cc2)cc1 ; CC1(C)c2ccccc2-c2ccc(N(c3ccccc3)c3ccc(-c4ccc(-c5cccc6sc7c(-c8cccc9ccccc89)cc8ccccc8c7c56)cc4)cc3)cc21]

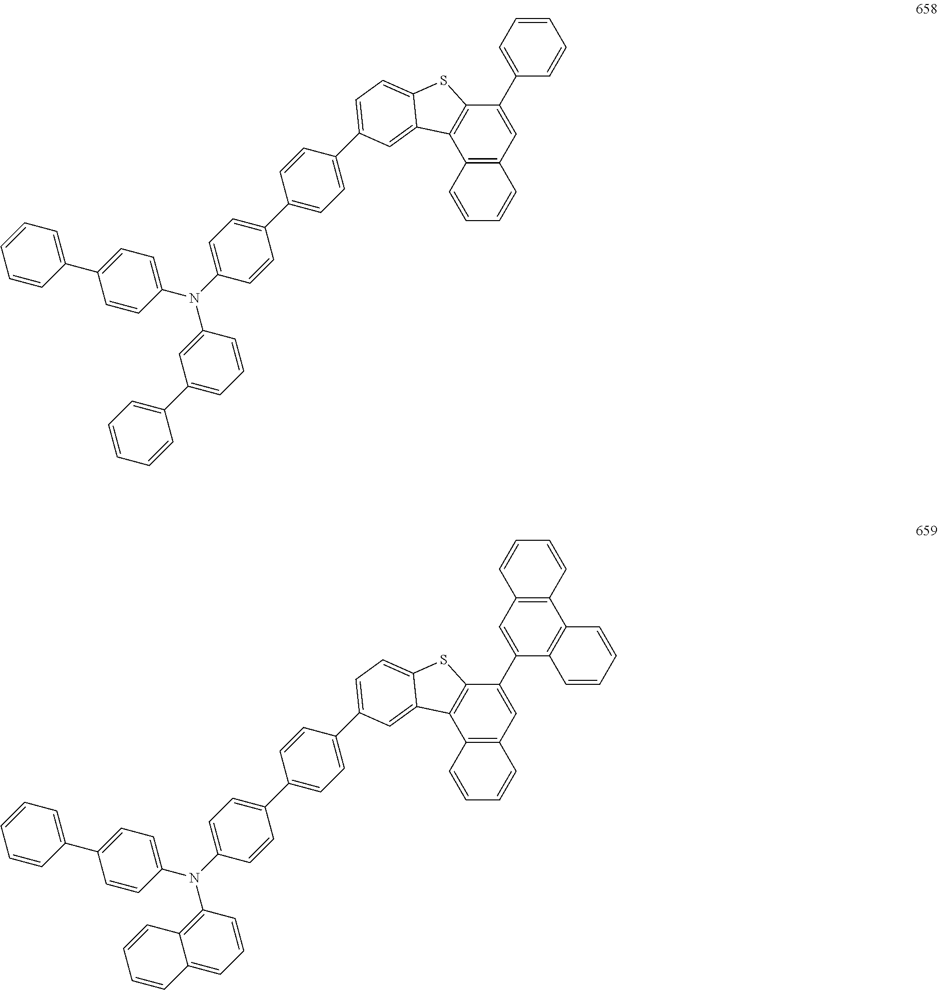 [SMILES: c1ccc(-c2ccc(N(c3ccc(-c4ccc(-c5ccc6sc7c(-c8ccccc8)cc8ccccc8c7c6c5)cc4)cc3)c3cccc(-c4ccccc4)c3)cc2)cc1 ; c1ccc(-c2ccc(N(c3ccc(-c4ccc(-c5ccc6sc7c(-c8cc9ccccc9c9ccccc89)cc8ccccc8c7c6c5)cc4)cc3)c3cccc4ccccc34)cc2)cc1]

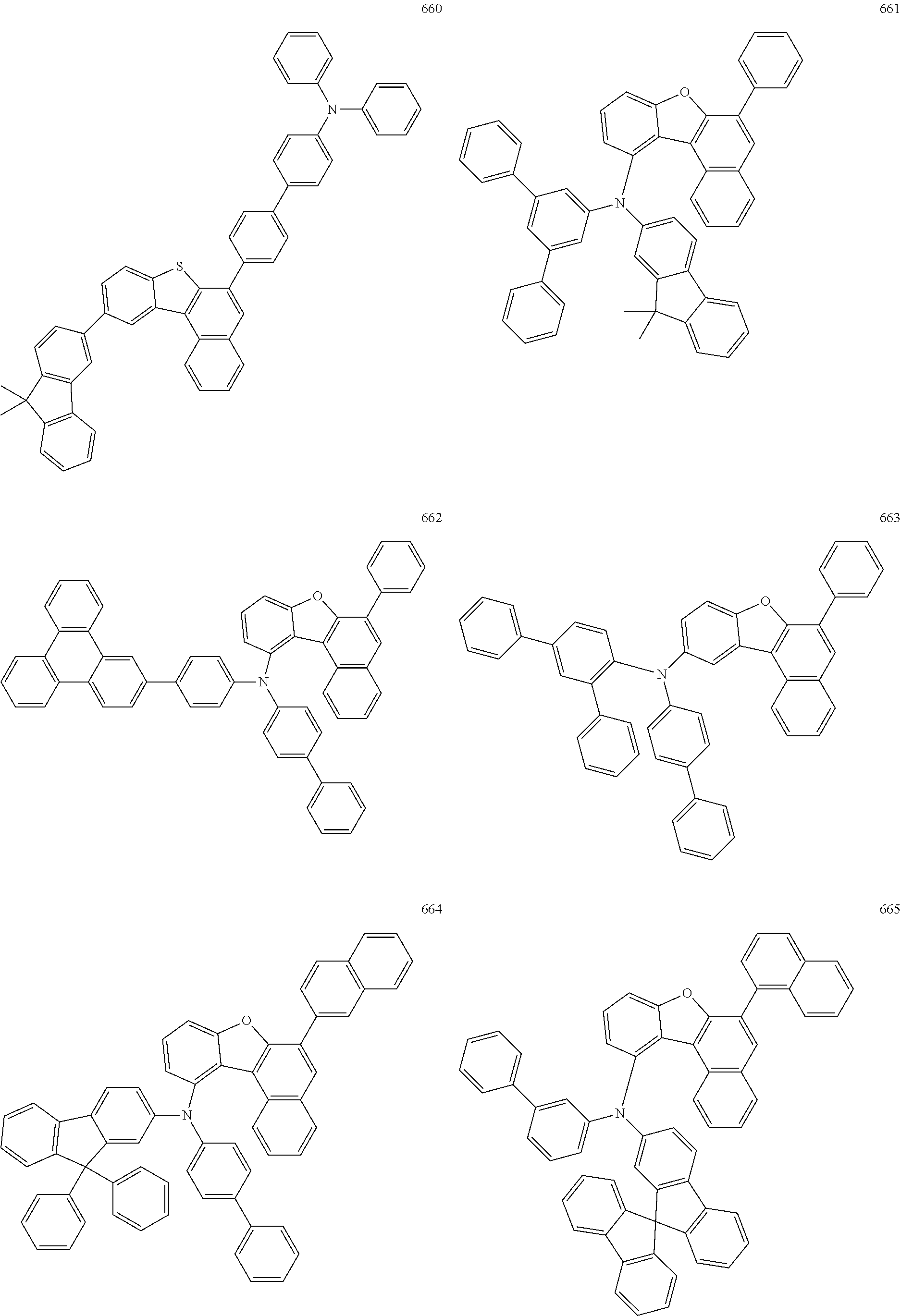

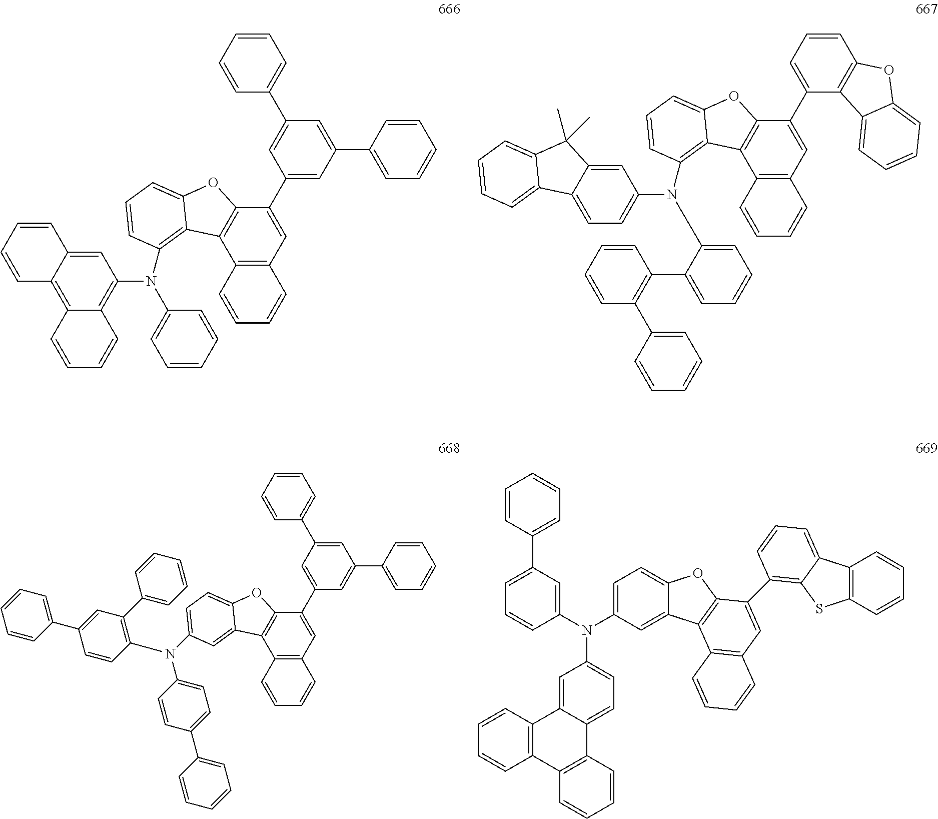

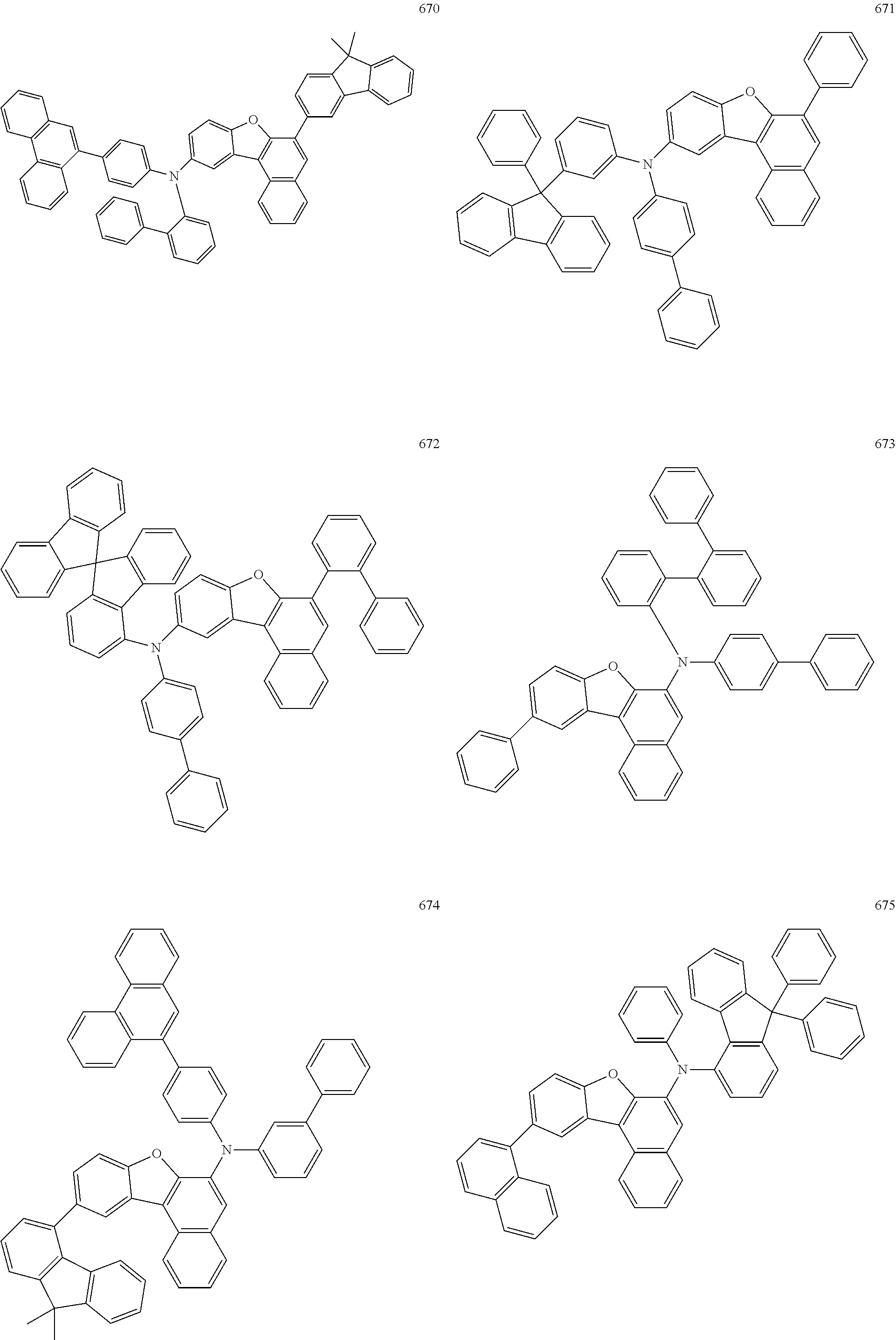

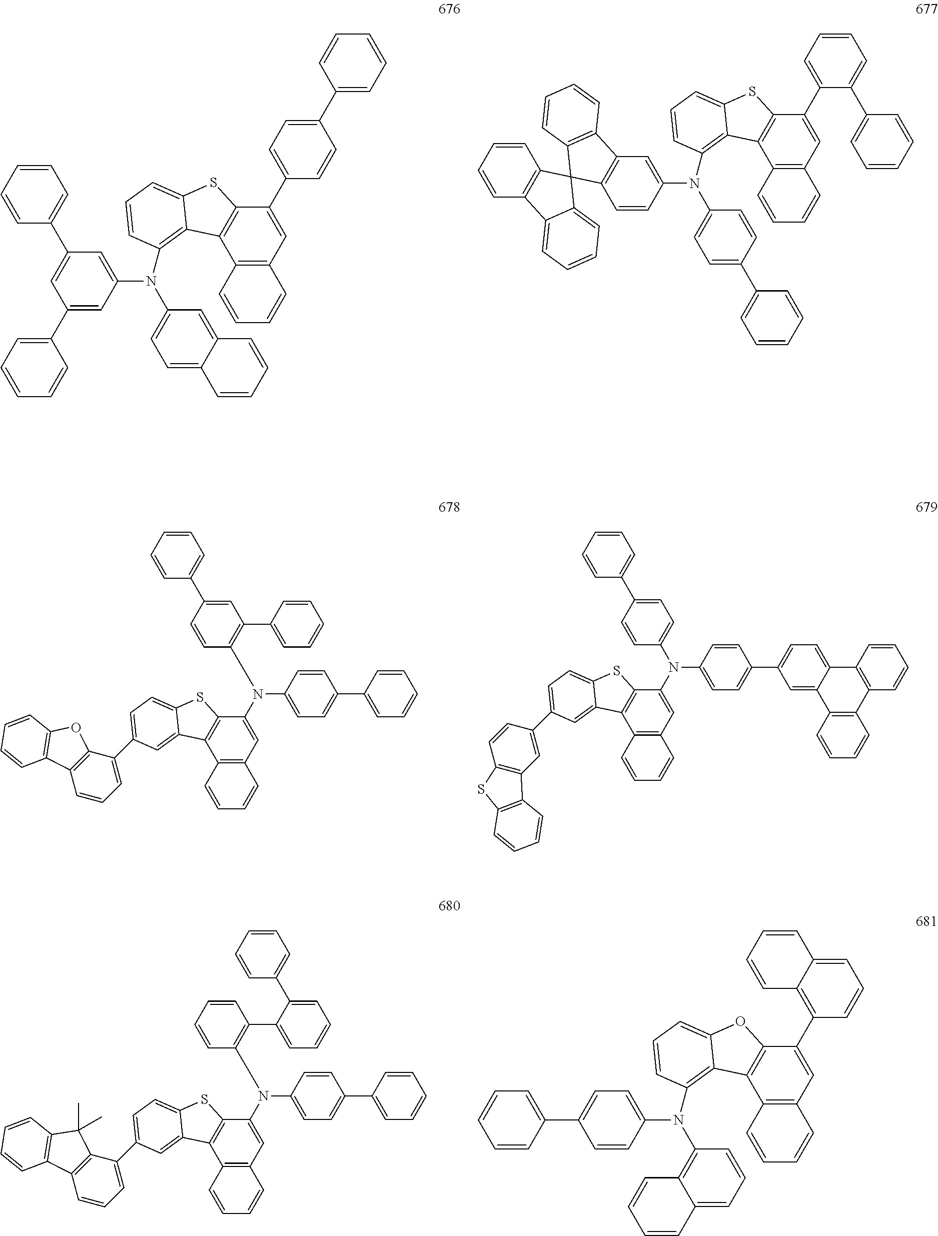

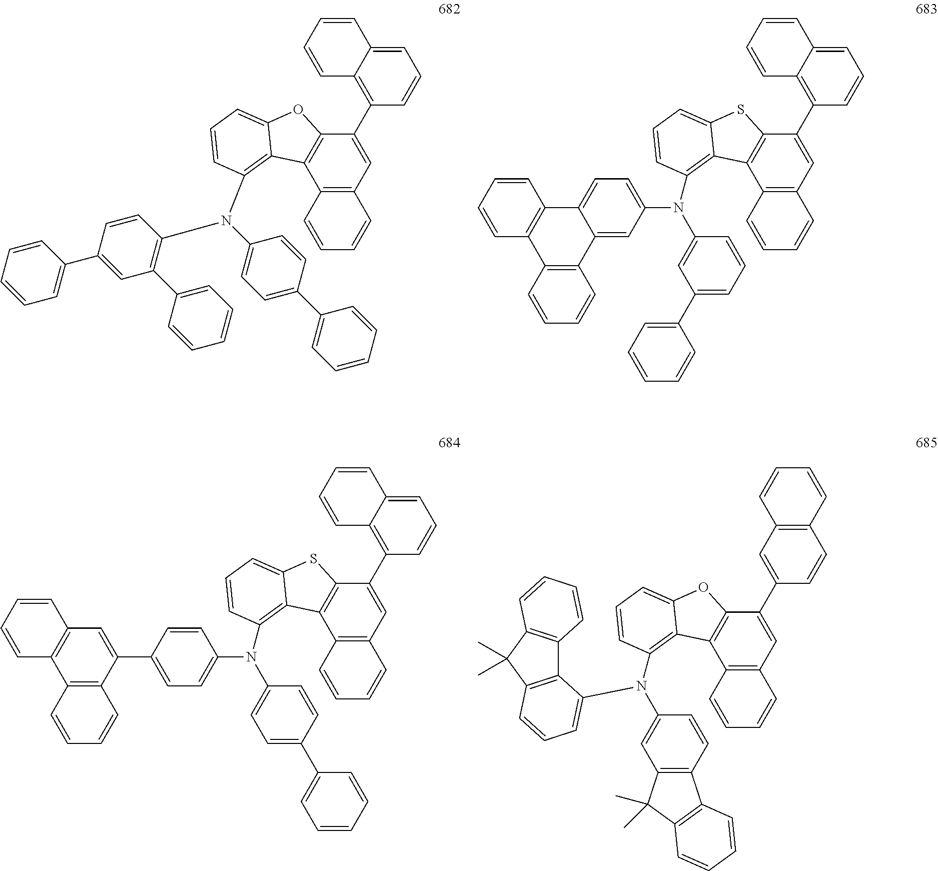 [SMILES: c1ccc(-c2ccc(N(c3ccc(-c4ccccc4)cc3-c3ccccc3)c3cccc4oc5c(-c6cccc7ccccc67)cc6ccccc6c5c34)cc2)cc1 ; c1ccc(-c2cccc(N(c3ccc4c5ccccc5c5ccccc5c4c3)c3cccc4sc5c(-c6cccc7ccccc67)cc6ccccc6c5c34)c2)cc1 ; c1ccc(-c2ccc(N(c3ccc(-c4cc5ccccc5c5ccccc45)cc3)c3cccc4sc5c(-c6cccc7ccccc67)cc6ccccc6c5c34)cc2)cc1 ; CC1(C)c2ccccc2-c2ccc(N(c3cccc4c3-c3ccccc3C4(C)C)c3cccc4oc5c(-c6ccc7ccccc7c6)cc6ccccc6c5c34)cc21]

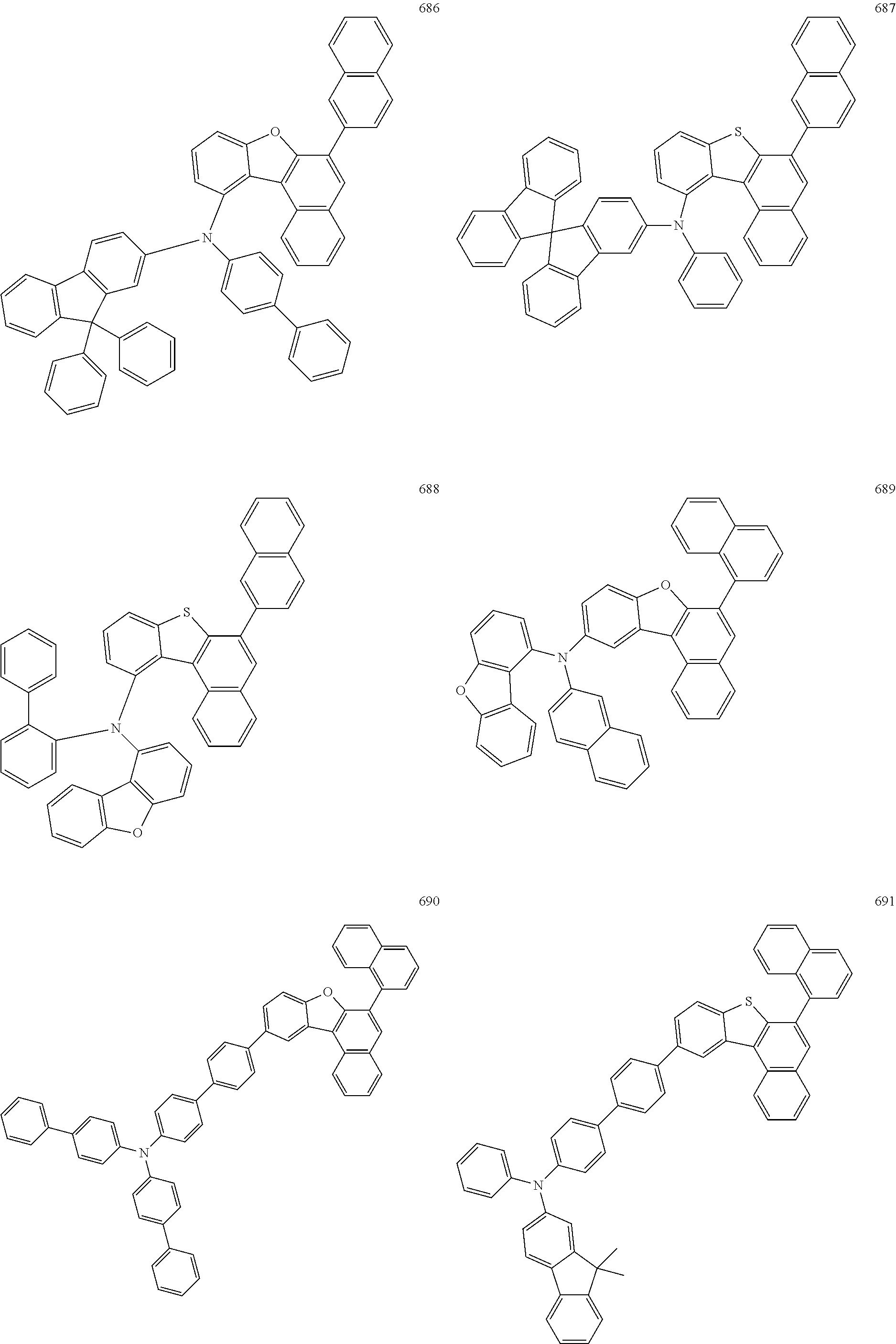

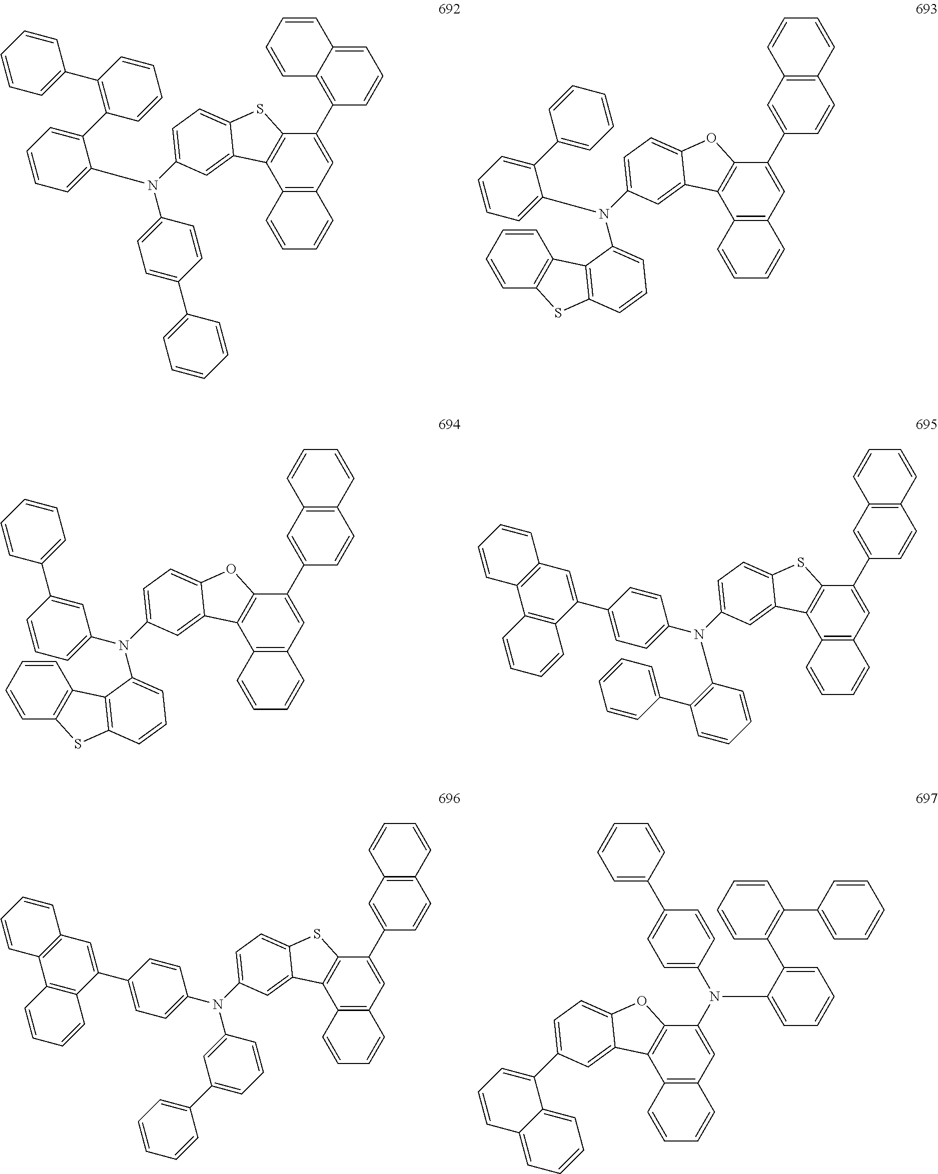

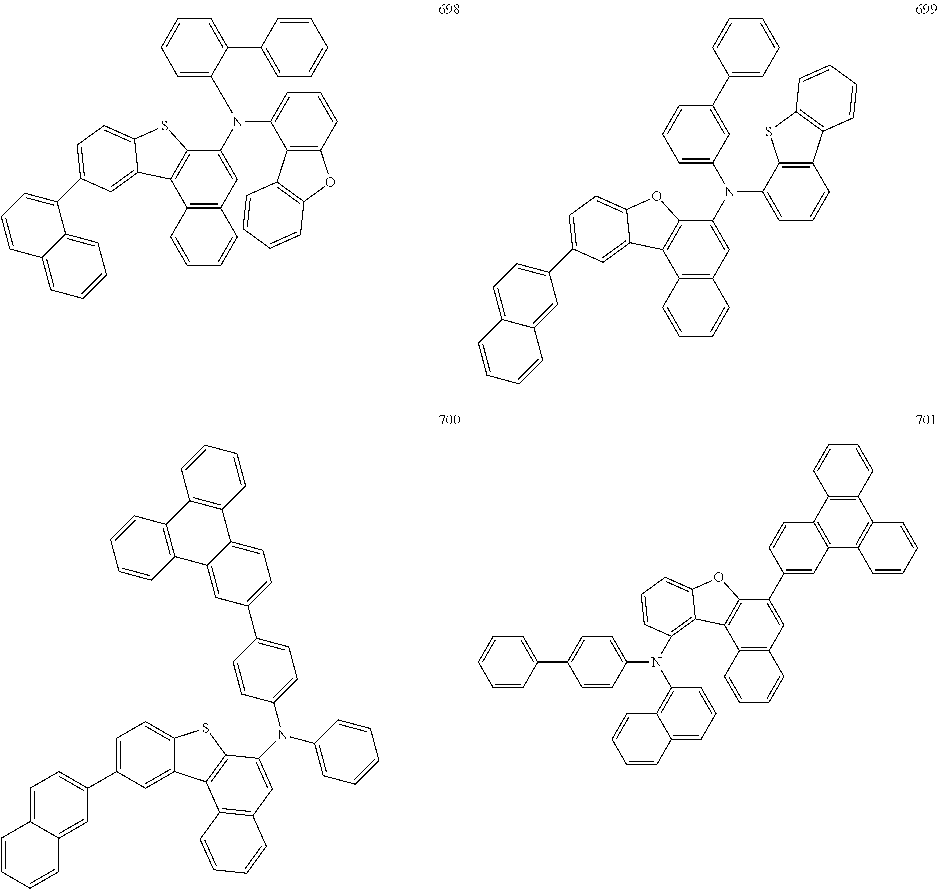 [SMILES: c1ccc(-c2ccccc2N(c2cc3ccccc3c3c2sc2ccc(-c4cccc5ccccc45)cc23)c2cccc3oc4ccccc4c23)cc1 ; c1ccc(-c2cccc(N(c3cc4ccccc4c4c3oc3ccc(-c5ccc6ccccc6c5)cc34)c3cccc4c3sc3ccccc34)c2)cc1 ; c1ccc(N(c2ccc(-c3ccc4c5ccccc5c5ccccc5c4c3)cc2)c2cc3ccccc3c3c2sc2ccc(-c4ccc5ccccc5c4)cc23)cc1 ; c1ccc(-c2ccc(N(c3cccc4ccccc34)c3cccc4oc5c(-c6ccc7c8ccccc8c8ccccc8c7c6)cc6ccccc6c5c34)cc2)cc1]

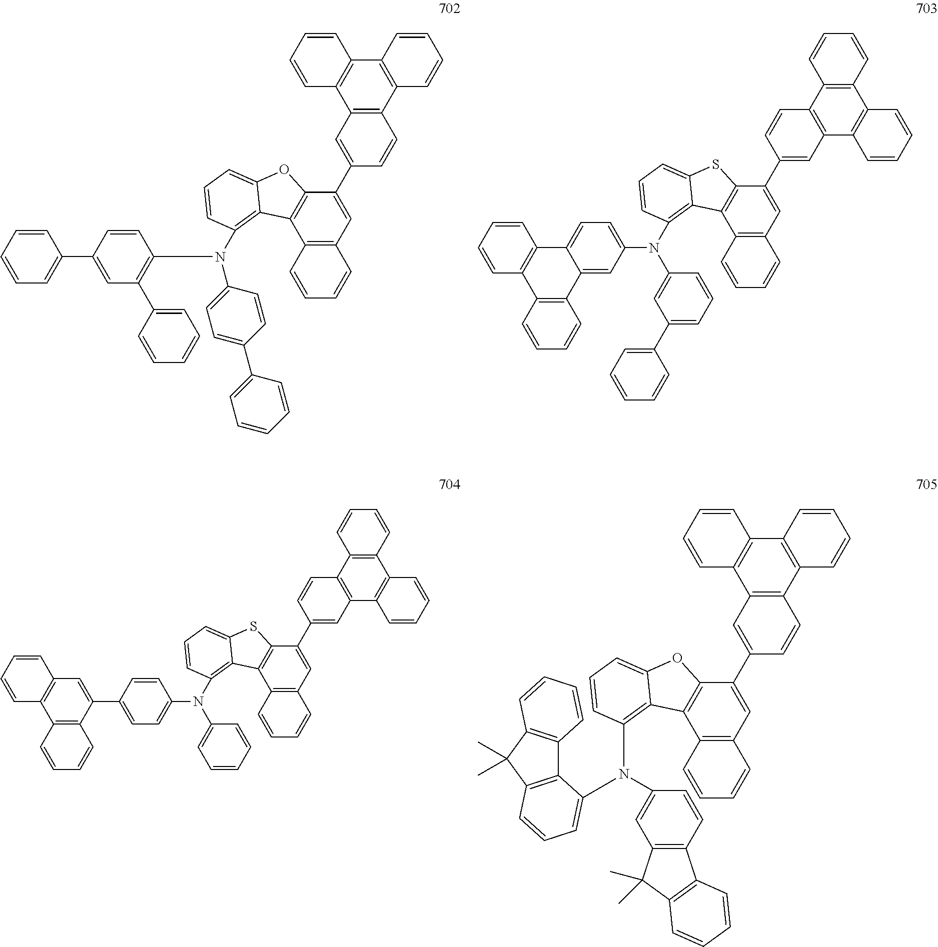 [SMILES: c1ccc(-c2ccc(N(c3ccc(-c4ccccc4)cc3-c3ccccc3)c3cccc4oc5c(-c6ccc7c8ccccc8c8ccccc8c7c6)cc6ccccc6c5c34)cc2)cc1 ; c1ccc(-c2cccc(N(c3ccc4c5ccccc5c5ccccc5c4c3)c3cccc4sc5c(-c6ccc7c8ccccc8c8ccccc8c7c6)cc6ccccc6c5c34)c2)cc1 ; c1ccc(N(c2ccc(-c3cc4ccccc4c4ccccc34)cc2)c2cccc3sc4c(-c5ccc6c7ccccc7c7ccccc7c6c5)cc5ccccc5c4c23)cc1 ; CC1(C)c2ccccc2-c2ccc(N(c3cccc4c3-c3ccccc3C4(C)C)c3cccc4oc5c(-c6ccc7c8ccccc8c8ccccc8c7c6)cc6ccccc6c5c34)cc21]

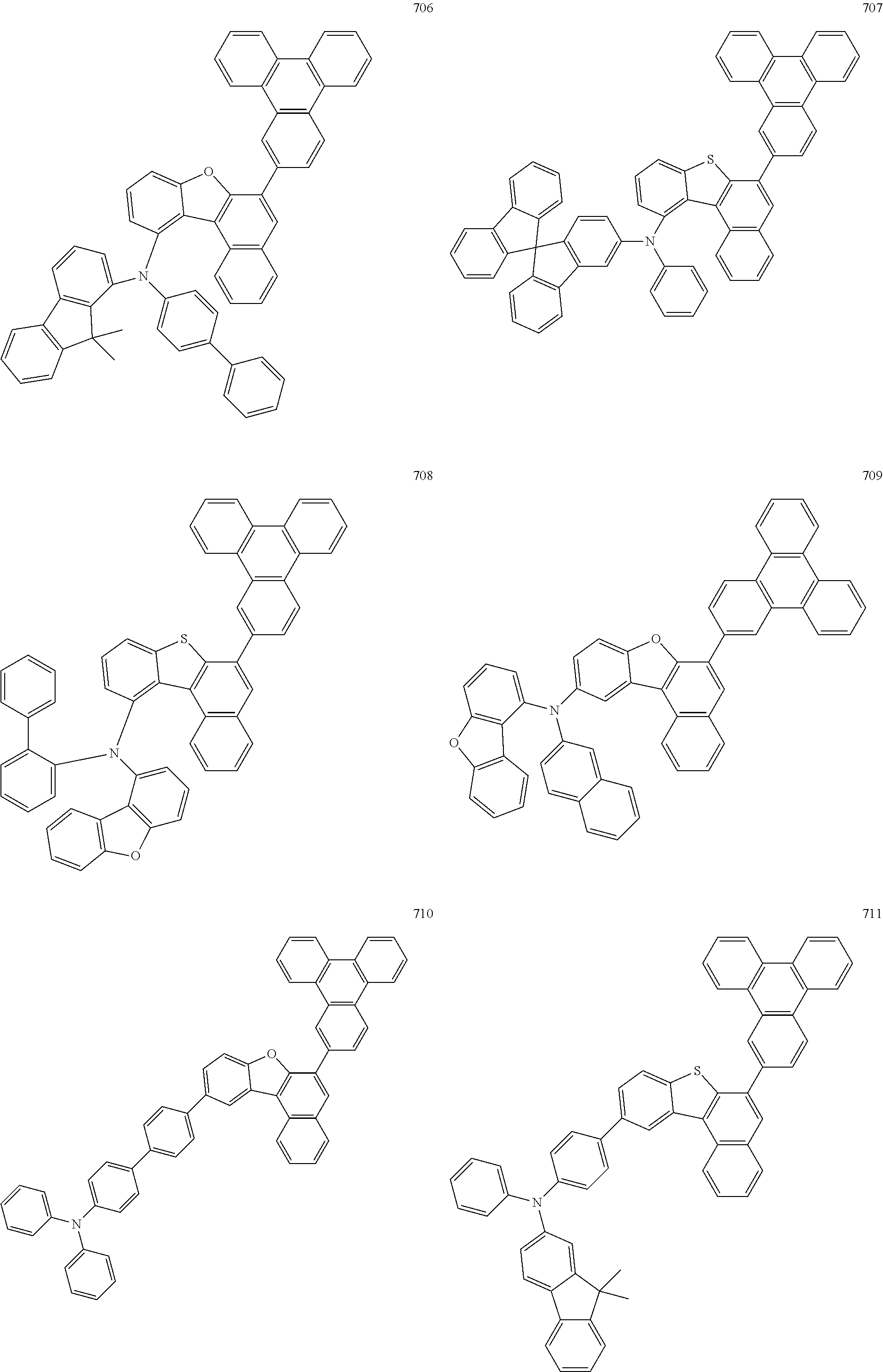

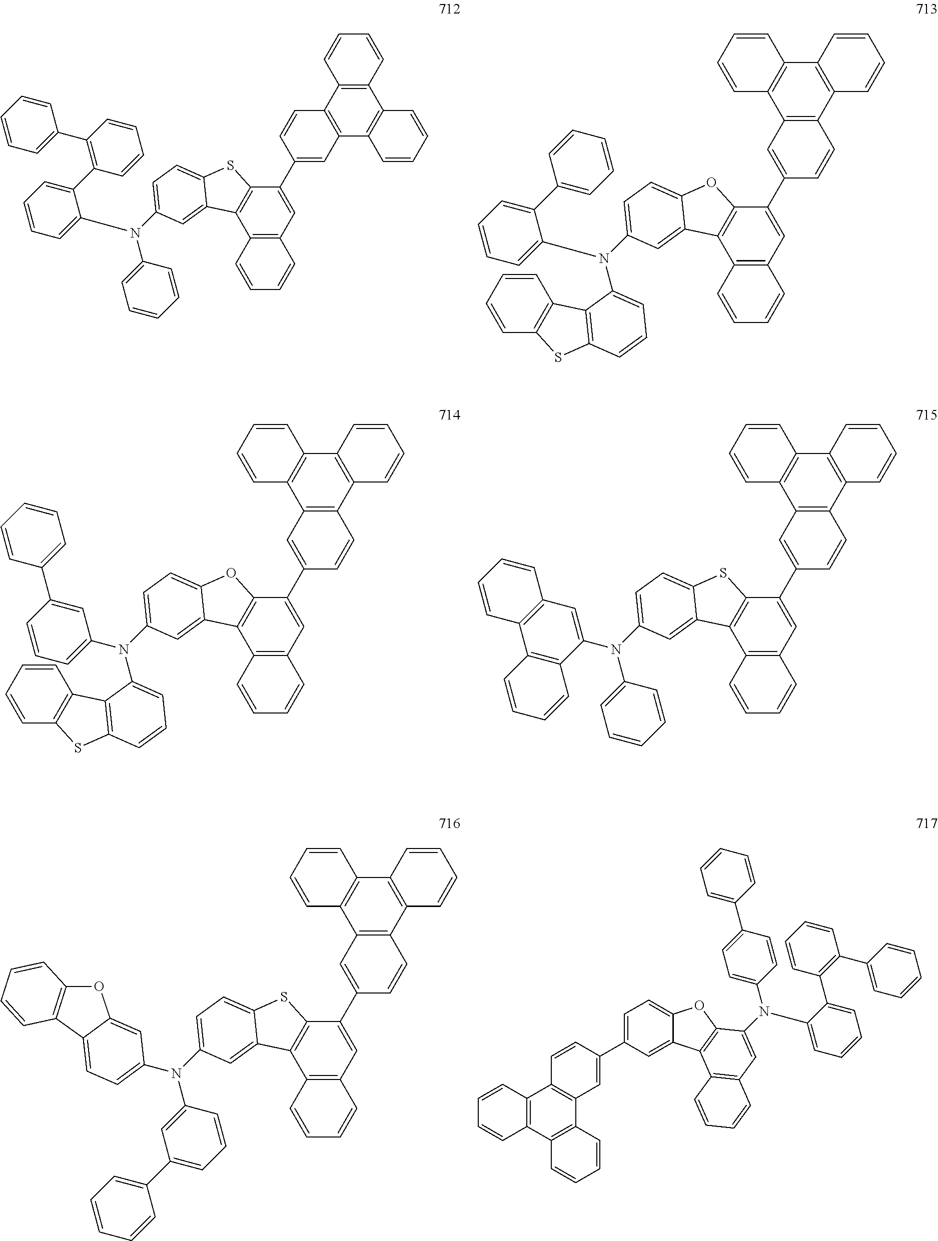

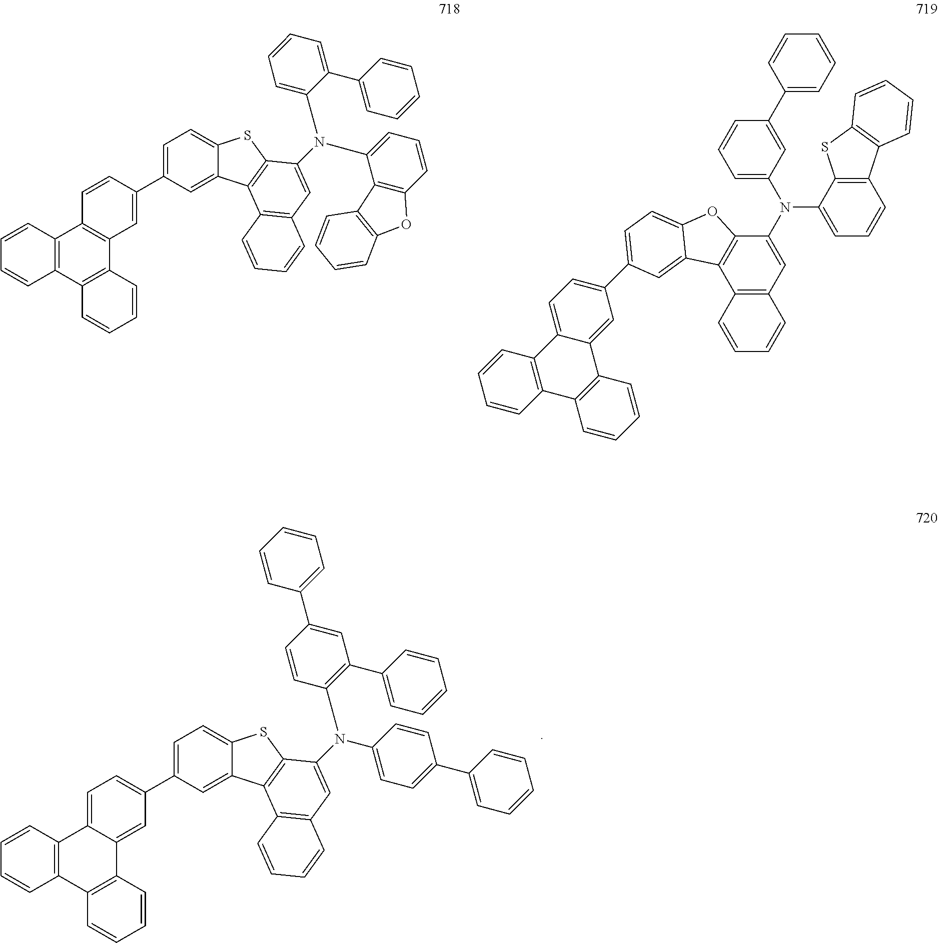 [SMILES: c1ccc(-c2ccccc2N(c2cc3ccccc3c3c2sc2ccc(-c4ccc5c6ccccc6c6ccccc6c5c4)cc23)c2cccc3oc4ccccc4c23)cc1 ; c1ccc(-c2cccc(N(c3cc4ccccc4c4c3oc3ccc(-c5ccc6c7ccccc7c7ccccc7c6c5)cc34)c3cccc4c3sc3ccccc34)c2)cc1 ; c1ccc(-c2ccc(N(c3ccc(-c4ccccc4)cc3-c3ccccc3)c3cc4ccccc4c4c3sc3ccc(-c5ccc6c7ccccc7c7ccccc7c6c5)cc34)cc2)cc1]

6. An organic light emitting device comprising:
a first electrode;
a second electrode provided to face the first electrode; and
an organic material layer having one or more layers provided between the first electrode and the second electrode,
wherein one or more layers of the organic material layer comprise one or more of the heterocyclic compound according to claim 1.

7. The organic light emitting device of claim 6, wherein the organic material layer comprises a light emitting layer, and the light emitting layer comprises the heterocyclic compound.

8. The organic light emitting device of claim 6, wherein the organic material layer comprises a hole transport layer or hole auxiliary layer, and the hole transport layer or hole auxiliary layer comprises the heterocyclic compound.

9. The organic light emitting device of claim 6, further comprising one or two or more layers selected from the group consisting of a light emitting layer, a hole injection layer, a hole transport layer, an electron injection layer, an electron transport layer, an electron blocking layer and a hole blocking layer.
